SEQUENCE LISTING (225 sequences)

SEQ ID NO: 1 - RNA, 45 nt, unidentified
  misc_feature 1..45: note = source = /note="Description of Unknown:GluR2 sequence"
  source 1..45: mol_type = other RNA
gtggaatagtataacaatatgctaaatgttgttatagtatcccac

SEQ ID NO: 2 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - RNA, 3583 nt, Homo sapiens
  source 1..3583: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 4 - RNA, 3526 nt, Homo sapiens
  source 1..3526: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 5 - RNA, 3358 nt, Homo sapiens
  source 1..3358: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 6 - RNA, 3572 nt, Homo sapiens
  source 1..3572: mol_type = other RNA
aaatagcacagccttgctgtgcgtggtagaagttgggttagtgttgacatgctgttgactcaccctcccgaggatggaagctctggcctgggtcaagttgtggtcactgcagttaacagtttgttgatctcagggagtattccacagttgctgatgtaattgacaatgattggagccagctcttccccagattcaaatggaccaattagaggacttgttggttctgtttatcaactatgtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 7 - RNA, 3190 nt, Homo sapiens
  source 1..3190: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 8 - RNA, 3415 nt, Homo sapiens
  source 1..3415: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 9 - RNA, 3295 nt, Homo sapiens
  source 1..3295: mol_type = other RNA
gtcggatgattcaagctcacggggacgagcaggagcgctctcgacttttctagagcctcagcgtcctaggactcacctttccctgatcctgcaccgtccctctcctggccccagactctccctcccactgttcacgaagcccaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 10 - RNA, 3529 nt, Homo sapiens
  source 1..3529: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 11 - RNA, 3472 nt, Homo sapiens
  source 1..3472: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 12 - RNA, 3304 nt, Homo sapiens
  source 1..3304: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 13 - RNA, 3415 nt, Homo sapiens
  source 1..3415: mol_type = other RNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagtgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 14 - protein, 40 aa, Homo sapiens
DAEFRHDSGYEVHHQKLVFFAEDVGSNKGAIIGLMVGGVV

SEQ ID NO: 15 - protein, 42 aa, Homo sapiens [sequence omitted]

SEQ ID NO: 16 - RNA, 6590 nt, Homo sapiens
  source 1..6590: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 17 - RNA, 5639 nt, Homo sapiens
  source 1..5639: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 18 - RNA, 5465 nt, Homo sapiens
  source 1..5465: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 19 - RNA, 5372 nt, Homo sapiens
  source 1..5372: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 20 - RNA, 5552 nt, Homo sapiens
  source 1..5552: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 21 - RNA, 6644 nt, Homo sapiens
  source 1..6644: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 22 - RNA, 5459 nt, Homo sapiens
  source 1..5459: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 23 - RNA, 5546 nt, Homo sapiens
  source 1..5546: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 24 - RNA, 6815 nt, Homo sapiens
  source 1..6815: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 25 - RNA, 6524 nt, Homo sapiens
  source 1..6524: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 26 - RNA, 4139 nt, Homo sapiens
  source 1..4139: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 27 - RNA, 5525 nt, Homo sapiens
  source 1..5525: mol_type = other RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 28 - protein, 862 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 29 - protein, 831 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 30 - protein, 804 aa, Homo sapiens
MAEPRQEFEVMEDHAGTYGLGDRKDQGGYTMHQDQEGDTDAGLKAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 31 - protein, 796 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 32 - protein, 765 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 33 - protein, 507 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 34 - protein, 478 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 35 - protein, 449 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 36 - RNA, 3177 nt, Homo sapiens
  source 1..3177: mol_type = other RNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 37 - RNA, 3396 nt, Homo sapiens
  source 1..3396: mol_type = other RNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 38 - RNA, 3030 nt, Homo sapiens
  source 1..3030: mol_type = other RNA
gcttctccattctggtgtgatccaggaacagctgtcttccagctctgaaagagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 39 - RNA, 3312 nt, Homo sapiens
  source 1..3312: mol_type = other RNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 40 - RNA, 3099 nt, Homo sapiens
  source 1..3099: mol_type = other RNA
gcttctccattctggtgtgatccaggaacagctgtcttccagctctgaaagagggctgagagattaggctgcttctccgggatccgcttttccccgggaaacgcgaggatgctccatggagctgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 41 - RNA, 3041 nt, Homo sapiens
  source 1..3041: mol_type = other RNA
ggcgacgaccagaaggggcccaagagagggggcgagcgaccgagcgccgcgacgcggaagtgagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 42 - RNA, 3465 nt, Homo sapiens
  source 1..3465: mol_type = other RNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagggctgagagattaggctgcttctccgggatccgcttttccccgggaaacgcgaggatgctccatggagctgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 43 - RNA, 3091 nt, Homo sapiens
  source 1..3091: mol_type = other RNA
gagtgtgagcggcgcctgctcagggtagatagctgagggcgggggtggatgttggatggattagaaccatcacacttgggcctgctgtttgcctgagtttgaaccacaccccgatgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 44 - RNA, 2984 nt, Homo sapiens
  source 1..2984: mol_type = other RNA
gctctaattctctgcaccttctcaagcattgtgcagattggttttctggattatcagcctgaaggacaaaacgaagaaacagccattagctcctgtctcccattgtctgagagctgccactaggatattaacttcctgaaattctgcagaaatctcctcttactttggcactggagatgcccatacgcagaaagcaaaaaggcacagcatatttaaggaagctcataagaaacagtgcatccagaagtggcgagaattggaggaatggacatgagactctaagaaccagcgcctttgatgttccttttgatctgttatgtagctcttcttgtacacagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 45 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 46 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 47 - protein, 92 aa, Homo sapiens
MPIRRKQKGTAYLRKLIRNSASRSGENWRNGHETLRTSAFDVPFDLLCSSSCTQNEEGAPQEGILEDMPVDPDNEAYEMPSEEGYQDYEPEA

SEQ ID NO: 48 - DNA, 90 bp, synthetic construct
  misc_feature 1..90: note = source = /note="Description of Unknown:APP target sequence"
  source 1..90: mol_type = other DNA
tatcaagacggaggagatctctgaagtgaagatggatgcagaattccgacatgactcaggatatgaagttcatcatcaaaaattggtgtt

SEQ ID NO: 49 - DNA, 20 bp, synthetic construct
  misc_feature 1..20: note = source = /note="Description of Artificial Sequence: Syntheticoligonucleotide"
  source 1..20: mol_type = other DNA
tctgcacccatcttcacttc

SEQ ID NO: 50 - DNA, 60 bp, synthetic construct
  misc_feature 1..60: note = source = /note="Description of Artificial Sequence: Syntheticoligonucleotide"
  source 1..60: mol_type = other DNA
tcatatcctgagtcatgtcggaattctgcacccatcttcacttcagagatctcctccgtc

SEQ ID NO: 51 - DNA, 60 bp, synthetic construct
  misc_feature 1..60: note = source = /note="Description of Artificial Sequence: Syntheticoligonucleotide"
  source 1..60: mol_type = other DNA
tcatatcctgagtcatgccggaattctgcacccatcttcacttcagagacctcctccgtc

SEQ ID NO: 52 - DNA, 105 bp, synthetic construct
  misc_feature 46..105: note = /note="This region may encompass one of the following sequences:'tctgcacccatcttcacttc' or 'tcatatcctga gtcatgtcggaattctgcacccatcttcacttcagagatctcctccgtc' or 'tcatatcctgagtcatgccggaattctgcacccatcttcacttcagagacctcctcc gtc'"
  misc_feature 1..105: note = source = /note="Description of Artificial Sequence: Syntheticpolynucleotide"
  source 1..105: mol_type = other DNA
gtggaatagtataacaatatgctaaatgttgttatagtatcccacnnnnnnnnnnnnnnnnnnnnnnnnnnnnnnnnnnnnnnnnnnnnnnnnnnnnnnnnnnnn

SEQ ID NO: 53 - DNA, 150 bp, synthetic construct
  misc_feature 46..105: note = /note="This region may encompass one of the following sequences:'tctgcacccatcttcacttc' or 'tcatatcctga gtcatgtcggaattctgcacccatcttcacttcagagatctcctccgtc' or 'tcatatcctgagtcatgccggaattctgcacccatcttcacttcagagacctcctcc gtc'"
  misc_feature 1..150: note = source = /note="Description of Artificial Sequence: Syntheticpolynucleotide"
  source 1..150: mol_type = other DNA
gtggaatagtataacaatatgctaaatgttgttatagtatcccacnnnnnnnnnnnnnnnnnnnnnnnnnnnnnnnnnnnnnnnnnnnnnnnnnnnnnnnnnnnngtggaatagtataacaatatgctaaatgttgttatagtatcccac

SEQ ID NO: 54 - DNA, 323 bp, synthetic construct
  misc_feature 117..176: note = /note="This region may encompass one of the following sequences:'tctgcacccatcttcacttc' or 'tcatatcctga gtcatgtcggaattctgcacccatcttcacttcagagatctcctccgtc' or 'tcatatcctgagtcatgccggaattctgcacccatcttcacttcagagacctcctcc gtc'"
  misc_feature 1..323: note = source = /note="Description of Artificial Sequence: Syntheticpolynucleotide"
  source 1..323: mol_type = other DNA
ggccgggcgcggtggctcacgcctgtaatcccagcactttgggaggccgaggcggggagattgcttgagcccaggagttcgagaccagcctgggcaacatagcgagaccccgtctcnnnnnnnnnnnnnnnnnnnnnnnnnnnnnnnnnnnnnnnnnnnnnnnnnnnnnnnnnnnnagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctc

SEQ ID NO: 55 - DNA, 5905 bp, synthetic construct
  misc_feature 1..5905: note = source = /note="Description of Artificial Sequence: Syntheticpolynucleotide"
  source 1..5905: mol_type = other DNA
ctcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgcggccgcatcgataatgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactgatgtcgacgatatctccagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccccgagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgaccggtgctacttgtacagctcgtccatgccgccggtggagtgtctaccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcttgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaattgaggggacaggatgtcccaggcgaagggcagggggccacccttggtcaccttcagcttggcggtctgggtgccctcgtaggggcgaccttcaccctcgccctcgatctcgaactcgtggccgttcacggagccctccatgtgcaccttgaagcgcatgaactccttgatgatggccatgttatcctcctcgcccttagaaaccatctccaggcgatctgacggttcactaaacgagctctgcttatataggcctcccaccgtacacgccacctcgacata

SEQ ID NO: 56 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaggatggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 57 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtggagatggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 58 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtggggatggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 59 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgagggtggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 60 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtggaggtggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 61 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgggggtggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 62 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaaggtggatgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 63 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatgggtgcagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 64 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgtggaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 65 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatacagaattccgacatgactcaggatatgaagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 66 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatgaagttcatcatcaaagattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 67 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatgaagttcatcatcaagaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 68 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatgaagttcatcatcaaggattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 69 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatgaagttcgtcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 70 - RNA, 2088 nt, synthetic construct
  misc_feature 1..2088: note = source = /note="Description of Artificial Sequence: Syntheticpolynucleotide"
  source 1..2088: mol_type = other RNA
atgctccctggacttgctttgctgcttttggcagcctggactgctcgagcactcgaggtcccaacggatggaaacgcgggtcttttggcagagcctcaaatagcaatgttttgcggaagactcaacatgcatatgaacgttcagaatgggaaatgggactccgaccccagtggtacgaagacatgtattgacacaaaggagggaatactccagtactgccaggaagtgtacccggagcttcagattacgaatgtggtagaggctaatcaacccgtaactatccaaaattggtgtaagagaggcaggaagcaatgcaagactcatcctcatttcgtaattccgtatcgatgtttggtgggagaatttgtctctgacgcattgcttgttcctgacaagtgtaagtttcttcaccaggaacgcatggacgtgtgcgagacacacttgcactggcataccgttgcgaaggagacgtgttccgaaaagagtacaaatctccatgactacggcatgttgctcccgtgcggaatagataagttccgaggcgtggagtttgtatgctgtccgctggcagaggagagcgataatgtcgattccgcagatgccgaagaggacgacagcgacgtctggtggggaggagcggacactgattacgctgatggtagtgaggacaaagtagtcgaggtggcagaagaagaagaagtggcggaggttgaagaagaagaggcagacgatgacgaagacgatgaggacggtgatgaggtagaagaagaagcggaagaaccgtacgaagaagctacggaacgcactacaagtattgctaccactacaaccactacaaccgaatcagttgaggaagtggtgcgagtccccactacggctgccagtacaccggatgccgtcgacaaatacctggagactcctggcgacgaaaacgaacatgctcatttccagaaggcgaaggaacgcctcgaagcaaagcacagagagagaatgtcacaggtaatgagggaatgggaggaggcggaacgccaagcaaagaacctgcctaaagcggacaagaaggcagttatccaacatttccaagagaaagtggagagtctcgaacaggaggcagcgaacgagaggcaacaattggtagaaacgcacatggcgagggtggaagctatgctcaatgaccgaagacgacttgccttggaaaattacattactgcccttcaagccgtcccaccgcgcccacgccatgtctttaacatgcttaagaagtatgttcgagctgaacagaaggatcggcaacacaccctgaaacacttcgaacatgtcagaatggttgacccgaagaaggctgcacagattcgaagtcaagttatgacccatttgagggtaatatatgagagaatgaaccaaagtctgagccttctctacaatgtccccgctgtggccgaggaaattcaggacgaagtcgatgagctcctgcaaaaggagcagaactactctgacgatgtacttgctaatatgatttcagagccaaggatcagttatggaaacgacgccctgatgcctagtcttaccgaaaccaagactacggtagaactccttcccgttaacggagagttcagcttggacgaccttcagccttggcactcattcggagctgattccgtaccagccaatacggagaatgaagtagagcccgtagacgcaagacctgcagcggacagagggctgacgacgagacccggtagcggtttgacaaatatcaagacggaggagatctctgaagtgaagatggatgcagaattccgacatgactcaggatatggagttcatcatcaaaaattggtgttctttgcagaagatgtcggttctaacaagggtgctatcataggccttatggtgggtggcgtcgtgattgcgaccgtgatagttattacgcttgtcatgctgaagaagaaacagtatacgtccatccatcacggtgtggtagaggtagatgcggccgtaactcccgaagagcgccatctttctaagatgcagcagaatggatacgagaaccccacgtacaaattctttgagcaaatgcaaaactga

SEQ ID NO: 71 - RNA, 289 nt, synthetic construct
  misc_feature 1..289: note = source = /note="Description of Artificial Sequence: Syntheticpolynucleotide"
  source 1..289: mol_type = other RNA
ggtgctcgcttcggcagcacatatactttgtgaaagaaggacgggtcaccttgtctttcctgctgcttctgccacaccctgtttggtcttctcagcagcagccacaactccctccttggcctttgaaagtcctttcacgaatacatccacggctaatgaattcctttacaccacactgtcgtcgaatggccactcccagttctccgctcacgagggtggaaaggcagaaggcttgaaggcaaggcgtgagtggcctgtgactaactgtgccaagcggacttcggtccgc

SEQ ID NO: 72 - RNA, 147 nt, synthetic construct
  misc_feature 1..147: note = source = /note="Description of Artificial Sequence: Syntheticpolynucleotide"
  source 1..147: mol_type = other RNA
gaacatcgtagattgaagccacaaaatccacagcacacaaagaccctgccaccatgtattcacttcagtgaaagggaagcaccgaaatgctgagtgggggcgtggaatttttggagcaggttttctgacttcggtcggaaaacccct

SEQ ID NO: 73 - RNA, 60 nt, synthetic construct
  misc_feature 1..60: note = source = /note="Description of Artificial Sequence: Syntheticoligonucleotide"
  source 1..60: mol_type = other RNA
aggagaaggagaaggaggaggactgggaggaggaggacggcgacgaccagaaggggccca

SEQ ID NO: 74 - RNA, 60 nt, synthetic construct
  misc_feature 1..60: note = source = /note="Description of Artificial Sequence: Syntheticoligonucleotide"
  source 1..60: mol_type = other RNA
cgacgaccagaaggggcccaagagagggggcgggcgaccgagcgccgcgacgcggaagtg

SEQ ID NO: 75 - RNA, 60 nt, synthetic construct
  misc_feature 1..60: note = source = /note="Description of Artificial Sequence: Syntheticoligonucleotide"
  source 1..60: mol_type = other RNA
agcgccgcgacgcggaagtgaggtgcgtgcgggctgcggcgcagaccccggcccggcccc

SEQ ID NO: 76 - RNA, 60 nt, synthetic construct
  misc_feature 1..60: note = source = /note="Description of Artificial Sequence: Syntheticoligonucleotide"
  source 1..60: mol_type = other RNA
gcagaccccggcccggcccctccgagggcgtcctgggcgctccctcacgccttgccttca

SEQ ID NO: 77 - RNA, 60 nt, synthetic construct
  misc_feature 1..60: note = source = /note="Description of Artificial Sequence: Syntheticoligonucleotide"
  source 1..60: mol_type = other RNA
tccctcacgccttgccttcaagccttctgcctttccgccctcgtgagcggagaactggga

SEQ ID NO: 78 - RNA, 60 nt, synthetic construct
  misc_feature 1..60: note = source = /note="Description of Artificial Sequence: Syntheticoligonucleotide"
  source 1..60: mol_type = other RNA
tcgtgagcggagaactgggagtggccattcgacgacggtgtggtgtaaaggaattcatta

SEQ ID NO: 79 - RNA, 60 nt, synthetic construct
  misc_feature 1..60: note = source = /note="Description of Artificial Sequence: Syntheticoligonucleotide"
  source 1..60: mol_type = other RNA
tggtgtaaaggaattcattagccgtggatgtattcatgaaaggactttcaaaggccgagg

SEQ ID NO: 80 - RNA, 60 nt, synthetic construct
  misc_feature 1..60: note = source = /note="Description of Artificial Sequence: Syntheticoligonucleotide"
  source 1..60: mol_type = other RNA
aggactttcaaaggccgaggagggagttgtggctgctgctgagaaatccaaacagggtgt

SEQ ID NO: 81 - RNA, 60 nt, synthetic construct
  misc_feature 1..60: note = source = /note="Description of Artificial Sequence: Syntheticoligonucleotide"
  source 1..60: mol_type = other RNA
gagaaatccaaacagggtgtggcagaagcagcaggaaagacaaatgagggtgttctctat

SEQ ID NO: 82 - RNA, 60 nt, synthetic construct
  misc_feature 1..60: note = source = /note="Description of Artificial Sequence: Syntheticoligonucleotide"
  source 1..60: mol_type = other RNA
caaatgagggtgttctctatgtaggctccgaaaccaaggagggagtggtgcatggtgtgg

SEQ ID NO: 83 - RNA, 60 nt, synthetic construct
  misc_feature 1..60: note = source = /note="Description of Artificial Sequence: Syntheticoligonucleotide"
  source 1..60: mol_type = other RNA
gggagtggtgcatggtgtggcaacggtggctgagaagaccaaagagcaagtgacaaatgt

SEQ ID NO: 84 - RNA, 60 nt, synthetic construct
  misc_feature 1..60: note = source = /note="Description of Artificial Sequence: Syntheticoligonucleotide"
  source 1..60: mol_type = other RNA
aaagagcaagtgacaaatgttggaggagcggtggtgacgggtgtgacagcagtagcccag

SEQ ID NO: 85 - RNA, 60 nt, synthetic construct
  misc_feature 1..60: note = source = /note="Description of Artificial Sequence: Syntheticoligonucleotide"
  source 1..60: mol_type = other RNA
gtgtgacagcagtagcccagaagacggtggagggagcagggagcattgcagcagccactg

SEQ ID NO: 86 - RNA, 60 nt, synthetic construct
  misc_feature 1..60: note = source = /note="Description of Artificial Sequence: Syntheticoligonucleotide"
  source 1..60: mol_type = other RNA
gagcattgcagcagccactggctttgtcaagaaggaccagttgggcaagaatgaagaagg

SEQ ID NO: 87 - RNA, 60 nt, synthetic construct
  misc_feature 1..60: note = source = /note="Description of Artificial Sequence: Syntheticoligonucleotide"
  source 1..60: mol_type = other RNA
ttgggcaagaatgaagaaggagccccacgggaaggaattctggaagatatgcctgtggat

SEQ ID NO: 88 - RNA, 60 nt, synthetic construct
  misc_feature 1..60: note = source = /note="Description of Artificial Sequence: Syntheticoligonucleotide"
  source 1..60: mol_type = other RNA
tggaagatatgcctgtggatcctgacgatgaggcttatgaaatgccttctgaggaagggt

SEQ ID NO: 89 - RNA, 60 nt, synthetic construct
  misc_feature 1..60: note = source = /note="Description of Artificial Sequence: Syntheticoligonucleotide"
  source 1..60: mol_type = other RNA
aatgccttctgaggaagggtatcaagactgcgaacctgaagcctaagaaatatctttgct

SEQ ID NO: 90 - RNA, 60 nt, synthetic construct
  misc_feature 1..60: note = source = /note="Description of Artificial Sequence: Syntheticoligonucleotide"
  source 1..60: mol_type = other RNA
gcctaagaaatatctttgctcccggtttcttgagatctgctgacagatgttccatcctgt

SEQ ID NO: 91 - RNA, 60 nt, synthetic construct
  misc_feature 1..60: note = source = /note="Description of Artificial Sequence: Syntheticoligonucleotide"
  source 1..60: mol_type = other RNA
tgacagatgttccatcctgtacaagtgctcggttccaatgtgcccagtcatgacatttct

SEQ ID NO: 92 - RNA, 60 nt, synthetic construct
  misc_feature 1..60: note = source = /note="Description of Artificial Sequence: Syntheticoligonucleotide"
  source 1..60: mol_type = other RNA
tgcccagtcatgacatttctcaaagtttttgcagtgtatctcgaagtcttccatcagcag

SEQ ID NO: 93 - RNA, 60 nt, synthetic construct
  misc_feature 1..60: note = source = /note="Description of Artificial Sequence: Syntheticoligonucleotide"
  source 1..60: mol_type = other RNA
tcgaagtcttccatcagcagtgattgaagtgtctgtacctgcccccactcagcatttcgg

SEQ ID NO: 94 - RNA, 60 nt, synthetic construct
  misc_feature 1..60: note = source = /note="Description of Artificial Sequence: Syntheticoligonucleotide"
  source 1..60: mol_type = other RNA
gcccccactcagcatttcggtgcttccctttcgctgaagtgaatacatggtagcagggtc

SEQ ID NO: 95 - RNA, 60 nt, synthetic construct
  misc_feature 1..60: note = source = /note="Description of Artificial Sequence: Syntheticoligonucleotide"
  source 1..60: mol_type = other RNA
gaatacatggtagcagggtctttgtgtgctgtgggttttgtggcttcaatctacgatgtt

SEQ ID NO: 96 - RNA, 60 nt, synthetic construct
  misc_feature 1..60: note = source = /note="Description of Artificial Sequence: Syntheticoligonucleotide"
  source 1..60: mol_type = other RNA
tggcttcaatctacgatgttaagacaaattaagaacacctaagtgactaccacttatttc

SEQ ID NO: 97 - RNA, 60 nt, synthetic construct
  misc_feature 1..60: note = source = /note="Description of Artificial Sequence: Syntheticoligonucleotide"
  source 1..60: mol_type = other RNA
aagtgactaccacttatttctaaatcctcgctatttttttgttgctgttgttcagaagtt

SEQ ID NO: 98 - RNA, 60 nt, synthetic construct
  misc_feature 1..60: note = source = /note="Description of Artificial Sequence: Syntheticoligonucleotide"
  source 1..60: mol_type = other RNA
gttgctgttgttcagaagttgttggtgatttgctatcatatattataagatttttaggtg

SEQ ID NO: 99 - RNA, 60 nt, synthetic construct
  misc_feature 1..60: note = source = /note="Description of Artificial Sequence: Syntheticoligonucleotide"
  source 1..60: mol_type = other RNA
tattataagatttttaggtgtcttttaatgatgctgtctaagaataatgacgtattgtga

SEQ ID NO: 100 - RNA, 60 nt, synthetic construct
  misc_feature 1..60: note = source = /note="Description of Artificial Sequence: Syntheticoligonucleotide"
  source 1..60: mol_type = other RNA
agaataatgacgtattgtgaaatttgttgatatatataatacttaataatatgtgagcat

SEQ ID NO: 101 - RNA, 60 nt, synthetic construct
  misc_feature 1..60: note = source = /note="Description of Artificial Sequence: Syntheticoligonucleotide"
  source 1..60: mol_type = other RNA
acttaataatatgtgagcatgaaactatgcgcctataaatactaaatatgaaattttacc

SEQ ID NO: 102 - RNA, 60 nt, synthetic construct
  misc_feature 1..60: note = source = /note="Description of Artificial Sequence: Syntheticoligonucleotide"
  source 1..60: mol_type = other RNA
actaaatatgaaattttaccgttttgcgatgtgttttattcacttgtgtttgtatataaa

SEQ ID NO: 103 - RNA, 60 nt, synthetic construct
  misc_feature 1..60: note = source = /note="Description of Artificial Sequence: Syntheticoligonucleotide"
  source 1..60: mol_type = other RNA
cacttgtgtttgtatataaatggtgagaattagaataatacgttatctcattgcataaat

SEQ ID NO: 104 - RNA, 60 nt, synthetic construct
  misc_feature 1..60: note = source = /note="Description of Artificial Sequence: Syntheticoligonucleotide"
  source 1..60: mol_type = other RNA
cgttatctcattgcataaatattttatttttatcccgtctcactttaataataataatca

SEQ ID NO: 105 - DNA, 60 bp, synthetic construct
  misc_feature 1..60: note = source = /note="Description of Artificial Sequence: Syntheticoligonucleotide"
  source 1..60: mol_type = other DNA
tgggccccttctggtcgtcgccgtcctcctcctcccagtcctcctccttctccttctcct

SEQ ID NO: 106 - DNA, 60 bp, synthetic construct
  misc_feature 1..60: note = source = /note="Description of Artificial Sequence: Syntheticoligonucleotide"
  source 1..60: mol_type = other DNA
cacttccgcgtcgcggcgctcggtcgcccgccccctctcttgggccccttctggtcgtcg

SEQ ID NO: 107 - DNA, 60 bp, synthetic construct
  misc_feature 1..60: note = source = /note="Description of Artificial Sequence: Syntheticoligonucleotide"
  source 1..60: mol_type = other DNA
acctcacttccgcgtcgcggcgctcggtcgctcgccccctctcttgggccccttctggtc

SEQ ID NO: 108 - DNA, 60 bp, synthetic construct
  misc_feature 1..60: note = source = /note="Description of Artificial Sequence: Syntheticoligonucleotide"
  source 1..60: mol_type = other DNA
ggggccgggccggggtctgcgccgcagcccgcacgcacctcacttccgcgtcgcggcgct

SEQ ID NO: 109 - DNA, 60 bp, synthetic construct
  misc_feature 1..60: note = source = /note="Description of Artificial Sequence: Syntheticoligonucleotide"
  source 1..60: mol_type = other DNA
ggacgctctcggaggggccgggccggggtctgcgccgcagcccgcacgcacctcacttcc

SEQ ID NO: 110 - DNA, 60 bp, synthetic construct
  misc_feature 1..60: note = source = /note="Description of Artificial Sequence: Syntheticoligonucleotide"
  source 1..60: mol_type = other DNA
tgaaggcaaggcgtgagggagcgcccaggacgccctcggaggggccgggccggggtctgc

SEQ ID NO: 111 - DNA, 60 bp, synthetic construct
  misc_feature 1..60: note = source = /note="Description of Artificial Sequence: Syntheticoligonucleotide"
  source 1..60: mol_type = other DNA
tcccagttctccgctcacgagggcggaaaggcagaaggcttgaaggcaaggcgtgaggga

SEQ ID NO: 112 - DNA, 60 bp, synthetic construct
  misc_feature 1..60: note = source = /note="Description of Artificial Sequence: Syntheticoligonucleotide"
  source 1..60: mol_type = other DNA
taatgaattcctttacaccacaccgtcgtcgaatggccactcccagttctccgctcacga

SEQ ID NO: 113 - DNA, 60 bp, synthetic construct
  misc_feature 1..60: note = source = /note="Description of Artificial Sequence: Syntheticoligonucleotide"
  source 1..60: mol_type = other DNA
cctcggcctttgaaagtcctttcatgaatacatccacggctaatgaattcctttacacca

SEQ ID NO: 114 - DNA, 60 bp, synthetic construct
  misc_feature 1..60: note = source = /note="Description of Artificial Sequence: Syntheticoligonucleotide"
  source 1..60: mol_type = other DNA
acaccctgtttggatttctcagcagcagccacaactccctcctcggcctttgaaagtcct

SEQ ID NO: 115 - DNA, 60 bp, synthetic construct
  misc_feature 1..60: note = source = /note="Description of Artificial Sequence: Syntheticoligonucleotide"
  source 1..60: mol_type = other DNA
atagagaacaccctcatttgtctttcctgctgcttctgccacaccctgtttggatttctc

SEQ ID NO: 116 - DNA, 60 bp, synthetic construct
  misc_feature 1..60: note = source = /note="Description of Artificial Sequence: Syntheticoligonucleotide"
  source 1..60: mol_type = other DNA
ccacaccatgcaccactccctccttggtttcggagcctacatagagaacaccctcatttg

SEQ ID NO: 117 - DNA, 60 bp, synthetic construct
  misc_feature 1..60: note = source = /note="Description of Artificial Sequence: Syntheticoligonucleotide"
  source 1..60: mol_type = other DNA
acatttgtcacttgctctttggtcttctcagccaccgttgccacaccatgcaccactccc

SEQ ID NO: 118 - DNA, 60 bp, synthetic construct
  misc_feature 1..60: note = source = /note="Description of Artificial Sequence: Syntheticoligonucleotide"
  source 1..60: mol_type = other DNA
ctgggctactgctgtcacacccgtcaccaccgctcctccaacatttgtcacttgctcttt

SEQ ID NO: 119 - DNA, 60 bp, synthetic construct
  misc_feature 1..60: note = source = /note="Description of Artificial Sequence: Syntheticoligonucleotide"
  source 1..60: mol_type = other DNA
cagtggctgctgcaatgctccctgctccctccaccgtcttctgggctactgctgtcacac

SEQ ID NO: 120 - DNA, 60 bp, synthetic construct
  misc_feature 1..60: note = source = /note="Description of Artificial Sequence: Syntheticoligonucleotide"
  source 1..60: mol_type = other DNA
ccttcttcattcttgcccaactggtccttcttgacaaagccagtggctgctgcaatgctc

SEQ ID NO: 121 - DNA, 60 bp, synthetic construct
  misc_feature 1..60: note = source = /note="Description of Artificial Sequence: Syntheticoligonucleotide"
  source 1..60: mol_type = other DNA
atccacaggcatatcttccagaattccttcccgtggggctccttcttcattcttgcccaa

SEQ ID NO: 122 - DNA, 60 bp, synthetic construct
  misc_feature 1..60: note = source = /note="Description of Artificial Sequence: Syntheticoligonucleotide"
  source 1..60: mol_type = other DNA
acccttcctcagaaggcatttcataagcctcatcgtcaggatccacaggcatatcttcca

SEQ ID NO: 123 - DNA, 60 bp, synthetic construct
  misc_feature 1..60: note = source = /note="Description of Artificial Sequence: Syntheticoligonucleotide"
  source 1..60: mol_type = other DNA
agcaaagatatttcttaggcttcaggttcgcagtcttgatacccttcctcagaaggcatt

SEQ ID NO: 124 - DNA, 60 bp, synthetic construct
  misc_feature 1..60: note = source = /note="Description of Artificial Sequence: Syntheticoligonucleotide"
  source 1..60: mol_type = other DNA
acaggatggaacatctgtcagcagatctcaagaaaccgggagcaaagatatttcttaggc

SEQ ID NO: 125 - DNA, 60 bp, synthetic construct
  misc_feature 1..60: note = source = /note="Description of Artificial Sequence: Syntheticoligonucleotide"
  source 1..60: mol_type = other DNA
agaaatgtcatgactgggcacattggaaccgagcacttgtacaggatggaacatctgtca

SEQ ID NO: 126 - DNA, 60 bp, synthetic construct
  misc_feature 1..60: note = source = /note="Description of Artificial Sequence: Syntheticoligonucleotide"
  source 1..60: mol_type = other DNA
ctgctgatggaagacttcgagatacactgcaaaaactttgagaaatgtcatgactgggca

SEQ ID NO: 127 - DNA, 60 bp, synthetic construct
  misc_feature 1..60: note = source = /note="Description of Artificial Sequence: Syntheticoligonucleotide"
  source 1..60: mol_type = other DNA
ccgaaatgctgagtgggggcaggtacagacacttcaatcactgctgatggaagacttcga

SEQ ID NO: 128 - DNA, 60 bp, synthetic construct
  misc_feature 1..60: note = source = /note="Description of Artificial Sequence: Syntheticoligonucleotide"
  source 1..60: mol_type = other DNA
gaccctgctaccatgtattcacttcagcgaaagggaagcaccgaaatgctgagtgggggc

SEQ ID NO: 129 - DNA, 60 bp, synthetic construct
  misc_feature 1..60: note = source = /note="Description of Artificial Sequence: Syntheticoligonucleotide"
  source 1..60: mol_type = other DNA
aacatcgtagattgaagccacaaaacccacagcacacaaagaccctgctaccatgtattc

SEQ ID NO: 130 - DNA, 60 bp, synthetic construct
  misc_feature 1..60: note = source = /note="Description of Artificial Sequence: Syntheticoligonucleotide"
  source 1..60: mol_type = other DNA
gaaataagtggtagtcacttaggtgttcttaatttgtcttaacatcgtagattgaagcca

SEQ ID NO: 131 - DNA, 60 bp, synthetic construct
  misc_feature 1..60: note = source = /note="Description of Artificial Sequence: Syntheticoligonucleotide"
  source 1..60: mol_type = other DNA
aacttctgaacaacagcaacaaaaaaatagcgaggatttagaaataagtggtagtcactt

SEQ ID NO: 132 - DNA, 60 bp, synthetic construct
  misc_feature 1..60: note = source = /note="Description of Artificial Sequence: Syntheticoligonucleotide"
  source 1..60: mol_type = other DNA
cacctaaaaatcttataatatatgatagcaaatcaccaacaacttctgaacaacagcaac

SEQ ID NO: 133 - DNA, 60 bp, synthetic construct
  misc_feature 1..60: note = source = /note="Description of Artificial Sequence: Syntheticoligonucleotide"
  source 1..60: mol_type = other DNA
tcacaatacgtcattattcttagacagcatcattaaaagacacctaaaaatcttataata

SEQ ID NO: 134 - DNA, 60 bp, synthetic construct
  misc_feature 1..60: note = source = /note="Description of Artificial Sequence: Syntheticoligonucleotide"
  source 1..60: mol_type = other DNA
atgctcacatattattaagtattatatatatcaacaaatttcacaatacgtcattattct

SEQ ID NO: 135 - DNA, 60 bp, synthetic construct
  misc_feature 1..60: note = source = /note="Description of Artificial Sequence: Syntheticoligonucleotide"
  source 1..60: mol_type = other DNA
ggtaaaatttcatatttagtatttataggcgcatagtttcatgctcacatattattaagt

SEQ ID NO: 136 - DNA, 60 bp, synthetic construct
  misc_feature 1..60: note = source = /note="Description of Artificial Sequence: Syntheticoligonucleotide"
  source 1..60: mol_type = other DNA
tttatatacaaacacaagtgaataaaacacatcgcaaaacggtaaaatttcatatttagt

SEQ ID NO: 137 - DNA, 60 bp, synthetic construct
  misc_feature 1..60: note = source = /note="Description of Artificial Sequence: Syntheticoligonucleotide"
  source 1..60: mol_type = other DNA
atttatgcaatgagataacgtattattctaattctcaccatttatatacaaacacaagtg

SEQ ID NO: 138 - DNA, 60 bp, synthetic construct
  misc_feature 1..60: note = source = /note="Description of Artificial Sequence: Syntheticoligonucleotide"
  source 1..60: mol_type = other DNA
tgattattattattaaagtgagacgggataaaaataaaatatttatgcaatgagataacg

SEQ ID NO: 139 - DNA, 60 bp, synthetic construct
  misc_feature 1..60: note = source = /note="Description of Artificial Sequence: Syntheticoligonucleotide"
  source 1..60: mol_type = other DNA
gctgggggagtgggaggcaaacccgctaacccgtcgtcgaatggccactcccagttctcc

SEQ ID NO: 140 - DNA, 60 bp, synthetic construct
  misc_feature 1..60: note = source = /note="Description of Artificial Sequence: Syntheticoligonucleotide"
  source 1..60: mol_type = other DNA
gcaccaaactgacatttggggtttacctacccacatagagaacaccctcttgtgtctttc

SEQ ID NO: 141 - DNA, 60 bp, synthetic construct
  misc_feature 1..60: note = source = /note="Description of Artificial Sequence: Syntheticoligonucleotide"
  source 1..60: mol_type = other DNA
atctttggatataagcacaatggagcttacccgttgccacaccatgcaccactccctcct

SEQ ID NO: 142 - DNA, 60 bp, synthetic construct
  misc_feature 1..60: note = source = /note="Description of Artificial Sequence: Syntheticoligonucleotide"
  source 1..60: mol_type = other DNA
aaatgtaacacaaaacgtacacagccataccttgcccaactggtccttgttgacaaagcc

SEQ ID NO: 143 - DNA, 60 bp, synthetic construct
  misc_feature 1..60: note = source = /note="Description of Artificial Sequence: Syntheticoligonucleotide"
  source 1..60: mol_type = other DNA
ttgttagaaagattcagcttggactcctaccccagaaggcatttcataagcctcattgtc

SEQ ID NO: 144 - DNA, 60 bp, synthetic construct
  misc_feature 1..60: note = source = /note="Description of Artificial Sequence: Syntheticoligonucleotide"
  source 1..60: mol_type = other DNA
atccatggctaatgaattcctttacaccacaccggaaaacataaaatacactttgaatga

SEQ ID NO: 145 - DNA, 60 bp, synthetic construct
  misc_feature 1..60: note = source = /note="Description of Artificial Sequence: Syntheticoligonucleotide"
  source 1..60: mol_type = other DNA
tgcaccactccctccttggttttggagcccacaaaaacaaattcaagacataagtctcaa

SEQ ID NO: 146 - DNA, 60 bp, synthetic construct
  misc_feature 1..60: note = source = /note="Description of Artificial Sequence: Syntheticoligonucleotide"
  source 1..60: mol_type = other DNA
ttgtcacttgctctttggtcttctcagccactggtacaaataaagagcaacaacagatta

SEQ ID NO: 147 - DNA, 60 bp, synthetic construct
  misc_feature 1..60: note = source = /note="Description of Artificial Sequence: Syntheticoligonucleotide"
  source 1..60: mol_type = other DNA
attccttcctgtggggctccttcttcattccaatatttaaagtaagaagcacaaaaagaa

SEQ ID NO: 148 - DNA, 60 bp, synthetic construct
  misc_feature 1..60: note = source = /note="Description of Artificial Sequence: Syntheticoligonucleotide"
  source 1..60: mol_type = other DNA
cttcaggttcgtagtcttgatacccttcccaatattagaaaaatcaaaaagacagcacac

SEQ ID NO: 149 - DNA, 5632 bp, synthetic construct
  misc_feature 1..5632: note = source = /note="Description of Artificial Sequence: Syntheticpolynucleotide"
  source 1..5632: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagtaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgaacatcgtagatcgaagccacaaaacccacagcacacaaagaccctgccaccatgcattcacttcagcgaaagggaagcaccgaaatgccgagtgggggcgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagcgtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaggaaactcaccctaactgtaaagtaattgtgtgttttgagactataaatatcccttggagaaaagccttgtttggaattcatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 150 - RNA, 100 nt, unidentified
  misc_feature 1..100: note = source = /note="Description of Unknown:APP target sequence"
  source 1..100: mol_type = other RNA
accaggttctgggttgacaaatatcaagacggaggagatctctgaagtgaagatggatgcagaattccgacatgactcaggatatgaagttcatcatcaa

SEQ ID NO: 151 - RNA, 100 nt, unidentified
  misc_feature 1..100: note = source = /note="Description of Unknown:APP target sequence"
  source 1..100: mol_type = other RNA
tacaggttctgggttgacaaatatcaagacggaggagatctctgaagtgaagatggatgcagaattccgacatgactcaggatatgaagttcatcatcaa

SEQ ID NO: 152 - RNA, 90 nt, unidentified
  misc_feature 1..90: note = source = /note="Description of Unknown:APP target sequence"
  source 1..90: mol_type = other RNA
gttctgggttgacaaatatcaagacggaggagatctctgaagtgaagatggatgcagaattccgacatgactcaggatatgaagttcatc

SEQ ID NO: 153 - DNA, 90 bp, unidentified
  misc_feature 1..90: note = source = /note="Description of Unknown:APP target sequence"
  source 1..90: mol_type = other DNA
tctgggttgacaaatatcaagacggaggagatctctgaagtgaagatggatgcagaattccgacatgactcaggatatgaagttcatcat

SEQ ID NO: 154 - DNA, 90 bp, unidentified
  misc_feature 1..90: note = source = /note="Description of Unknown:APP target sequence"
  source 1..90: mol_type = other DNA
aggagatctctgaagtgaagatggatgcagaattccgacatgactcaggatatgaagttcatcatcaaaaattggtgttctttgcagaag

SEQ ID NO: 155 - DNA, 90 bp, unidentified
  misc_feature 1..90: note = source = /note="Description of Unknown:APP target sequence"
  source 1..90: mol_type = other DNA
tctgggttgacaaatatcaagacggaggagatctggaagtgaacatggatgaagaattccgacatgactcaggatatgaagttcatcatc

SEQ ID NO: 156 - DNA, 90 bp, unidentified
  misc_feature 1..90: note = source = /note="Description of Unknown:APP target sequence"
  source 1..90: mol_type = other DNA
aggagatctggaagtgaacatggatgaagaattccgacatgactcaggatatgaagttcatcatcaaaaattggtgttctttgcagaaga

SEQ ID NO: 157 - DNA, 60 bp, unidentified
  misc_feature 1..60: note = source = /note="Description of Unknown:APP target sequence"
  source 1..60: mol_type = other DNA
atcaagacggaggagatctggaagtgaacatggatgaagaattccgacatgactcaggat

SEQ ID NO: 158 - DNA, 60 bp, unidentified
  misc_feature 1..60: note = source = /note="Description of Unknown:APP target sequence"
  source 1..60: mol_type = other DNA
aggagatctggaagtgaacatggatgaagaattccgacatgactcaggatatgaagttca

SEQ ID NO: 159 - RNA, 100 nt, synthetic construct
  misc_feature 1..100: note = source = /note="Description of Artificial Sequence: Syntheticpolynucleotide"
  source 1..100: mol_type = other RNA
ttgatgatgaacttcatatcctgagtcatgtcggaattctgcatccatcctcacttcagagatctcctccgtcttgatatttgtcaacccagaacctggt

SEQ ID NO: 160 - RNA, 100 nt, synthetic construct
  misc_feature 1..100: note = source = /note="Description of Artificial Sequence: Syntheticpolynucleotide"
  source 1..100: mol_type = other RNA
ttgatgatgaacttcatatcctgagtcatgtcggaattctgcatccatcctcacttcagagatctcctccgtcttgatatttgtcaacccagaacctgta

SEQ ID NO: 161 - RNA, 90 nt, synthetic construct
  misc_feature 1..90: note = source = /note="Description of Artificial Sequence: Syntheticoligonucleotide"
  source 1..90: mol_type = other RNA
gatgaacttcatatcctgagtcatgtcggaattctgcatccatcctcacttcagagatctcctccgtcttgatatttgtcaacccagaac

SEQ ID NO: 162 - DNA, 90 bp, synthetic construct
  misc_feature 1..90: note = source = /note="Description of Artificial Sequence: Syntheticoligonucleotide"
  source 1..90: mol_type = other DNA
atgatgaacttcatatcctgagtcatgtcggaattctgcatccaccttcacttcagagatctcctccgtcttgatatttgtcaacccaga

SEQ ID NO: 163 - DNA, 90 bp, synthetic construct
  misc_feature 1..90: note = source = /note="Description of Artificial Sequence: Syntheticoligonucleotide"
  source 1..90: mol_type = other DNA
cttctgcaaagaacaccaatttttgatgatgaacttcatatcctgagtcatgtcggaattctgcatccaccttcacttcagagatctcct

SEQ ID NO: 164 - DNA, 90 bp, synthetic construct
  misc_feature 1..90: note = source = /note="Description of Artificial Sequence: Syntheticoligonucleotide"
  source 1..90: mol_type = other DNA
gatgatgaacttcatatcctgagtcatgtcggaattcttcatccacgttcacttccagatctcctccgtcttgatatttgtcaacccaga

SEQ ID NO: 165 - DNA, 90 bp, synthetic construct
  misc_feature 1..90: note = source = /note="Description of Artificial Sequence: Syntheticoligonucleotide"
  source 1..90: mol_type = other DNA
tcttctgcaaagaacaccaatttttgatgatgaacttcatatcctgagtcatgtcggaattcttcatccacgttcacttccagatctcct

SEQ ID NO: 166 - DNA, 60 bp, synthetic construct
  misc_feature 1..60: note = source = /note="Description of Artificial Sequence: Syntheticoligonucleotide"
  source 1..60: mol_type = other DNA
atcctgagtcatgtcggaattcttcatccacgttcacttccagatctcctccgtcttgat

SEQ ID NO: 167 - DNA, 60 bp, synthetic construct
  misc_feature 1..60: note = source = /note="Description of Artificial Sequence: Syntheticoligonucleotide"
  source 1..60: mol_type = other DNA
tgaacttcatatcctgagtcatgtcggaattcttcatccacgttcacttccagatctcct

SEQ ID NO: 168 - DNA, 5288 bp, synthetic construct
  misc_feature 1..5288: note = source = /note="Description of Artificial Sequence: Syntheticpolynucleotide"
  source 1..5288: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagagaccagctggatggtctcttttttttcgtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggatgtcttcacaggaagacgctttttttgcggccgcatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 169 - DNA, 20 bp, synthetic construct
  misc_feature 1..20: note = source = /note="Description of Artificial Sequence: Syntheticoligonucleotide"
  source 1..20: mol_type = other DNA
actctggcggcaagttctgc

SEQ ID NO: 170 - DNA, 30 bp, synthetic construct
  misc_feature 1..30: note = source = /note="Description of Artificial Sequence: Syntheticoligonucleotide"
  source 1..30: mol_type = other DNA
cactctggcggcaagttctgctcagcggag

SEQ ID NO: 171 - DNA, 40 bp, synthetic construct
  misc_feature 1..40: note = source = /note="Description of Artificial Sequence: Syntheticoligonucleotide"
  source 1..40: mol_type = other DNA
gcactctggcggcaagttctgctcagcggagtttctgccc

SEQ ID NO: 172 - DNA, 50 bp, synthetic construct
  misc_feature 1..50: note = source = /note="Description of Artificial Sequence: Syntheticoligonucleotide"
  source 1..50: mol_type = other DNA
agcactctggcggcaagttctgctcagcggagtttctgcccggccaaaca

SEQ ID NO: 173 - DNA, 75 bp, synthetic construct
  misc_feature 1..75: note = source = /note="Description of Artificial Sequence: Syntheticoligonucleotide"
  source 1..75: mol_type = other DNA
aggagcactctggcggcaagttctgctcagcggagtttctgcccggccaaacagcgtaaccccttcttccaagca

SEQ ID NO: 174 - DNA, 100 bp, synthetic construct
  misc_feature 1..100: note = source = /note="Description of Artificial Sequence: Syntheticpolynucleotide"
  source 1..100: mol_type = other DNA
ggaggagcactctggcggcaagttctgctcagcggagtttctgcccggccaaacagcgtaaccccttcttccaagcagatttctttgcagccaaatgcaa

SEQ ID NO: 175 - DNA, 150 bp, synthetic construct
  misc_feature 1..150: note = source = /note="Description of Artificial Sequence: Syntheticpolynucleotide"
  source 1..150: mol_type = other DNA
caacaggaggagcactctggcggcaagttctgctcagcggagtttctgcccggccaaacagcgtaaccccttcttccaagcagatttctttgcagccaaatgcaagggatgttaaggcaagaccctccccacagggcagtcagagacccg

SEQ ID NO: 176 - DNA, 200 bp, synthetic construct
  misc_feature 1..200: note = source = /note="Description of Artificial Sequence: Syntheticpolynucleotide"
  source 1..200: mol_type = other DNA
ctcagcaacaggaggagcactctggcggcaagttctgctcagcggagtttctgcccggccaaacagcgtaaccccttcttccaagcagatttctttgcagccaaatgcaagggatgttaaggcaagaccctccccacagggcagtcagagacccgcagccgacagactaagcctgcagcttccaaccaggctccccgaga

SEQ ID NO: 177 - DNA, 20 bp, synthetic construct
  misc_feature 1..20: note = source = /note="Description of Artificial Sequence: Syntheticoligonucleotide"
  source 1..20: mol_type = other DNA
aggaggagcactctggcggc

SEQ ID NO: 178 - DNA, 30 bp, synthetic construct
  misc_feature 1..30: note = source = /note="Description of Artificial Sequence: Syntheticoligonucleotide"
  source 1..30: mol_type = other DNA
gcaacaggaggagcactctggcggcaagtt

SEQ ID NO: 179 - DNA, 40 bp, synthetic construct
  misc_feature 1..40: note = source = /note="Description of Artificial Sequence: Syntheticoligonucleotide"
  source 1..40: mol_type = other DNA
actcagcaacaggaggagcactctggcggcaagttctgct

SEQ ID NO: 180 - DNA, 50 bp, synthetic construct
  misc_feature 1..50: note = source = /note="Description of Artificial Sequence: Syntheticoligonucleotide"
  source 1..50: mol_type = other DNA
atgatactcagcaacaggaggagcactctggcggcaagttctgctcagcg

SEQ ID NO: 181 - DNA, 75 bp, synthetic construct
  misc_feature 1..75: note = source = /note="Description of Artificial Sequence: Syntheticoligonucleotide"
  source 1..75: mol_type = other DNA
acgtggaggacgatgatactcagcaacaggaggagcactctggcggcaagttctgctcagcggagtttctgcccg

SEQ ID NO: 182 - DNA, 100 bp, synthetic construct
  misc_feature 1..100: note = source = /note="Description of Artificial Sequence: Syntheticpolynucleotide"
  source 1..100: mol_type = other DNA
caccagcaccgcgacgtggaggacgatgatactcagcaacaggaggagcactctggcggcaagttctgctcagcggagtttctgcccggccaaacagcgt

SEQ ID NO: 183 - DNA, 150 bp, synthetic construct
  misc_feature 1..150: note = source = /note="Description of Artificial Sequence: Syntheticpolynucleotide"
  source 1..150: mol_type = other DNA
tgacgatcgtggagacgaacagcagcaccagcaccgcgacgtggaggacgatgatactcagcaacaggaggagcactctggcggcaagttctgctcagcggagtttctgcccggccaaacagcgtaaccccttcttccaagcagatttct

SEQ ID NO: 184 - DNA, 200 bp, synthetic construct
  misc_feature 1..200: note = source = /note="Description of Artificial Sequence: Syntheticpolynucleotide"
  source 1..200: mol_type = other DNA
tgtccattgcccacgatccattggctgacgatcgtggagacgaacagcagcaccagcaccgcgacgtggaggacgatgatactcagcaacaggaggagcactctggcggcaagttctgctcagcggagtttctgcccggccaaacagcgtaaccccttcttccaagcagatttctttgcagccaaatgcaagggatgtta

SEQ ID NO: 185 - DNA, 20 bp, synthetic construct
  misc_feature 1..20: note = source = /note="Description of Artificial Sequence: Syntheticoligonucleotide"
  source 1..20: mol_type = other DNA
tcagcaacaggaggagcact

SEQ ID NO: 186 - DNA, 30 bp, synthetic construct
  misc_feature 1..30: note = source = /note="Description of Artificial Sequence: Syntheticoligonucleotide"
  source 1..30: mol_type = other DNA
cgatgatactcagcaacaggaggagcactc

SEQ ID NO: 187 - DNA, 40 bp, synthetic construct
  misc_feature 1..40: note = source = /note="Description of Artificial Sequence: Syntheticoligonucleotide"
  source 1..40: mol_type = other DNA
cgtggaggacgatgatactcagcaacaggaggagcactct

SEQ ID NO: 188 - DNA, 50 bp, synthetic construct
  misc_feature 1..50: note = source = /note="Description of Artificial Sequence: Syntheticoligonucleotide"
  source 1..50: mol_type = other DNA
gcaccgcgacgtggaggacgatgatactcagcaacaggaggagcactctg

SEQ ID NO: 189 - DNA, 75 bp, synthetic construct
  misc_feature 1..75: note = source = /note="Description of Artificial Sequence: Syntheticoligonucleotide"
  source 1..75: mol_type = other DNA
gtggagacgaacagcagcaccagcaccgcgacgtggaggacgatgatactcagcaacaggaggagcactctggcg

SEQ ID NO: 190 - DNA, 100 bp, synthetic construct
  misc_feature 1..100: note = source = /note="Description of Artificial Sequence: Syntheticpolynucleotide"
  source 1..100: mol_type = other DNA
ccacgatccattggctgacgatcgtggagacgaacagcagcaccagcaccgcgacgtggaggacgatgatactcagcaacaggaggagcactctggcggc

SEQ ID NO: 191 - DNA, 150 bp, synthetic construct
  misc_feature 1..150: note = source = /note="Description of Artificial Sequence: Syntheticpolynucleotide"
  source 1..150: mol_type = other DNA
agaggtgctacagttctgccagagatcagttgcgtgtccattgcccacgatccattggctgacgatcgtggagacgaacagcagcaccagcaccgcgacgtggaggacgatgatactcagcaacaggaggagcactctggcggcaagttc

SEQ ID NO: 192 - DNA, 100 bp, synthetic construct
  misc_feature 1..100: note = source = /note="Description of Artificial Sequence: Syntheticpolynucleotide"
  source 1..100: mol_type = other DNA
tgataaaaggcgtacataattcttgtgtctactgtacagaatactgccgccagctggatttcccaattctgagtaacactctgcaatccaaacagggttc

SEQ ID NO: 193 - protein, 6 aa, Homo sapiens
YENPTY

SEQ ID NO: 194 - protein, 6 aa, synthetic construct
  MOD_RES 2: note = Any amino acid
  REGION 1..6: note = source = /note="Description of Artificial Sequence: Syntheticpeptide"
KXKEGV

SEQ ID NO: 195 - protein, 9 aa, synthetic construct
  REGION 1..9: note = source = /note="Description of Artificial Sequence: Syntheticpeptide"
VGGAVVTGV

SEQ ID NO: 196 - protein, 6 aa, synthetic construct
  REGION 1..6: note = source = /note="Description of Artificial Sequence: Syntheticpeptide"
DPDNEA

SEQ ID NO: 197 - protein, 65 aa, Homo sapiens
KTEEISEVKMDAEFRHDSGYEVHHQKLVFFAEDVGSNKGAIIGLMVGGVVIATVIVITLVMLKKK

SEQ ID NO: 198 - protein, 12 aa, Homo sapiens
KTEEISEVKMDA

SEQ ID NO: 199 - protein, 59 aa, synthetic construct
  REGION 1..59: note = source = /note="Description of Artificial Sequence: Syntheticpolypeptide"
EVKMDAEFRHDSGYEVHHQKLVFFAEDVGSNKGAIIGLMVGGVVIATVIVITLVMLKKQ

SEQ ID NO: 200 - protein, 66 aa, Homo sapiens
EISEVKMDAEFRHDSGYEVHHQKLVFFAEDVGSNKGAIIGLMVGGVVIATVIVITLVMLKKKQYTS

SEQ ID NO: 201 - protein, 52 aa, unidentified
  VARIANT 6: note = /replace="Thr"
  VARIANT 15: note = /replace="Lys"
  SITE 1..52: note = /note="Variant residues given in the sequence have nopreference with respect to those in the annotationsfor variant positions"
  REGION 1..52: note = source = /note="Description of Unknown:APP sequence"
  REGION 1..52: note = source = /note="See specification as filed for detailed description ofsubstitutions and preferred embodiments"
EVKMDAEFRHDSGYEVHHQKLVFFAEDVGSNKGAIIGLMVGGVVIATVIVIT

SEQ ID NO: 202 - DNA, 23 bp, synthetic construct
  misc_feature 1..23: note = source = /note="Description of Artificial Sequence: Syntheticoligonucleotide"
  source 1..23: mol_type = other DNA
attagccatggatgtattcatga

SEQ ID NO: 203 - protein, 52 aa, unidentified
  VARIANT 3: note = /replace="Glu" or "Gly" or "Arg"
  VARIANT 4: note = /replace="Val"
  VARIANT 5: note = /replace="Gly"
  VARIANT 15: note = /replace="Gly"
  VARIANT 20: note = /replace="Glu" or "Gly" or "Arg"
  SITE 1..52: note = /note="Variant residues given in the sequence have nopreference with respect to those in the annotationsfor variant positions"
  REGION 1..52: note = source = /note="Description of Unknown:APP sequence"
  REGION 1..52: note = source = /note="See specification as filed for detailed description ofsubstitutions and preferred embodiments"
EVKMDAEFGHDSGFEVRHQKLVFFAEDVGSNKGAIIGLMVGGVVIATVIVIT

SEQ ID NO: 204 - DNA, 15 bp, synthetic construct
  misc_feature 1..15: note = source = /note="Description of Artificial Sequence: Syntheticoligonucleotide"
  source 1..15: mol_type = other DNA
tacatggtagcaggg

SEQ ID NO: 205 - DNA, 15 bp, synthetic construct
  misc_feature 1..15: note = source = /note="Description of Artificial Sequence: Syntheticoligonucleotide"
  source 1..15: mol_type = other DNA
tacatggtggcaggg

SEQ ID NO: 206 - DNA, 22 bp, synthetic construct
  misc_feature 1..22: note = source = /note="Description of Artificial Sequence: Syntheticoligonucleotide"
  source 1..22: mol_type = other DNA
gaagtgaatacatggtagcagg

SEQ ID NO: 207 - DNA, 70 bp, Homo sapiens
  source 1..70: mol_type = other DNA
tctgaagtgaagatggatgcagaattccgacatgactcaggatatgaagttcatcatcaaaaattggtgt

SEQ ID NO: 208 - protein, 23 aa, Homo sapiens
SEVKMDAEFRHDSGYEVHHQKLV

SEQ ID NO: 209 - DNA, 70 bp, synthetic construct
  misc_feature 1..70: note = source = /note="Description of Artificial Sequence: Syntheticoligonucleotide"
  source 1..70: mol_type = other DNA
tctgaagtgaggatggatgcagaattccgacatgactcaggatatgaagttcatcatcaaaaattggtgt

SEQ ID NO: 210 - DNA, 70 bp, synthetic construct
  misc_feature 1..70: note = source = /note="Description of Artificial Sequence: Syntheticoligonucleotide"
  source 1..70: mol_type = other DNA
tctgaagtggagatggatgcagaattccgacatgactcaggatatgaagttcatcatcaaaaattggtgt

SEQ ID NO: 211 - DNA, 70 bp, synthetic construct
  misc_feature 1..70: note = source = /note="Description of Artificial Sequence: Syntheticoligonucleotide"
  source 1..70: mol_type = other DNA
tctgaagtggggatggatgcagaattccgacatgactcaggatatgaagttcatcatcaaaaattggtgt

SEQ ID NO: 212 - DNA, 70 bp, synthetic construct
  misc_feature 1..70: note = source = /note="Description of Artificial Sequence: Syntheticoligonucleotide"
  source 1..70: mol_type = other DNA
tctgaagtgagggtggatgcagaattccgacatgactcaggatatgaagttcatcatcaaaaattggtgt

SEQ ID NO: 213 - DNA, 70 bp, synthetic construct
  misc_feature 1..70: note = source = /note="Description of Artificial Sequence: Syntheticoligonucleotide"
  source 1..70: mol_type = other DNA
tctgaagtggaggtggatgcagaattccgacatgactcaggatatgaagttcatcatcaaaaattggtgt

SEQ ID NO: 214 - DNA, 70 bp, synthetic construct
  misc_feature 1..70: note = source = /note="Description of Artificial Sequence: Syntheticoligonucleotide"
  source 1..70: mol_type = other DNA
tctgaagtgggggtggatgcagaattccgacatgactcaggatatgaagttcatcatcaaaaattggtgt

SEQ ID NO: 215 - DNA, 70 bp, synthetic construct
  misc_feature 1..70: note = source = /note="Description of Artificial Sequence: Syntheticoligonucleotide"
  source 1..70: mol_type = other DNA
tctgaagtgaaggtggatgcagaattccgacatgactcaggatatgaagttcatcatcaaaaattggtgt

SEQ ID NO: 216 - DNA, 70 bp, synthetic construct
  misc_feature 1..70: note = source = /note="Description of Artificial Sequence: Syntheticoligonucleotide"
  source 1..70: mol_type = other DNA
tctgaagtgaagatgggtgcagaattccgacatgactcaggatatgaagttcatcatcaaaaattggtgt

SEQ ID NO: 217 - DNA, 70 bp, synthetic construct
  misc_feature 1..70: note = source = /note="Description of Artificial Sequence: Syntheticoligonucleotide"
  source 1..70: mol_type = other DNA
tctgaagtgaagatggatgtggaattccgacatgactcaggatatgaagttcatcatcaaaaattggtgt

SEQ ID NO: 218 - DNA, 70 bp, synthetic construct
  misc_feature 1..70: note = source = /note="Description of Artificial Sequence: Syntheticoligonucleotide"
  source 1..70: mol_type = other DNA
tctgaagtgaagatggatacagaattccgacatgactcaggatatgaagttcatcatcaaaaattggtgt

SEQ ID NO: 219 - DNA, 82 bp, Homo sapiens
  source 1..82: mol_type = other DNA
ctctgaagtgaagatggatgcagaattccgacatgactcaggatatgaagttcatcatcaaaaattggtgttctttgcagaa

SEQ ID NO: 220 - protein, 27 aa, Homo sapiens
SEVKMDAEFRHDSGYEVHHQKLVFFAE

SEQ ID NO: 221 - DNA, 82 bp, synthetic construct
  misc_feature 1..82: note = source = /note="Description of Artificial Sequence: Syntheticoligonucleotide"
  source 1..82: mol_type = other DNA
ctctgaagtgaagatggatgcagaattccgacatgactcaggatatgaagttcatcatcaaagattggtgttctttgcagaa

SEQ ID NO: 222 - DNA, 82 bp, synthetic construct
  misc_feature 1..82: note = source = /note="Description of Artificial Sequence: Syntheticoligonucleotide"
  source 1..82: mol_type = other DNA
ctctgaagtgaagatggatgcagaattccgacatgactcaggatatgaagttcatcatcaagaattggtgttctttgcagaa

SEQ ID NO: 223 - DNA, 82 bp, synthetic construct
  misc_feature 1..82: note = source = /note="Description of Artificial Sequence: Syntheticoligonucleotide"
  source 1..82: mol_type = other DNA
ctctgaagtgaagatggatgcagaattccgacatgactcaggatatgaagttcatcatcaaggattggtgttctttgcagaa

SEQ ID NO: 224 - DNA, 82 bp, synthetic construct
  misc_feature 1..82: note = source = /note="Description of Artificial Sequence: Syntheticoligonucleotide"
  source 1..82: mol_type = other DNA
ctctgaagtgaagatggatgcagaattccgacatgactcaggatatgaagttcgtcatcaaaaattggtgttctttgcagaa

SEQ ID NO: 225 - DNA, 82 bp, synthetic construct
  misc_feature 1..82: note = source = /note="Description of Artificial Sequence: Syntheticoligonucleotide"
  source 1..82: mol_type = other DNA
ctctgaagtgaagatggatgcagaattccgacatgactcaggatatggagttcatcatcaaaaattggtgttctttgcagaa

(19) United States
(12) Patent Application Publication
(10) Pub. No.: US 2024/0279650 A1
(43) Pub. Date: Aug. 22, 2024

(54) Therapeutic Editing

(71) Applicant: Shape Therapeutics Inc., Seattle, WA (US)

(72) Inventors: David HUSS, Seattle, WA (US); Prashant MALI, San Diego, CA (US); Anupama LAKSHMANAN, Sammamish, WA (US); Christopher NYE, Issaquah, WA (US); Yiannis SAVVA, Seattle, WA (US); Liana STEIN, Kenmore, WA (US); Richard SULLIVAN, Seattle, WA (US); Rafael PONCE, Seattle, WA (US); Susan BYRNE, Lexington, MA (US)

(21) Appl. No.: 18/474,327

(22) Filed: Sep. 26, 2023

Related U.S. Application Data
(63) Continuation of application No. 17/781,877, filed on Jun. 2, 2022, now Pat. No. 11,827,880.
(60) Provisional application No. 63/112,286, filed on Nov. 11, 2020.
(60) Provisional application No. 63/030,165, filed on May 26, 2020.
(60) Provisional application No. 63/022,727, filed on May 11, 2020.
(60) Provisional application No. 62/942,683, filed on Dec. 2, 2019.
(60) Provisional application No. 62/942,667, filed on Dec. 2, 2019.
(60) Provisional application No. 62/942,693, filed on Dec. 2, 2019.

(51) Int. Cl. C12N 15/11; A61K 48/00; A61P 25/28; C07K 14/47; C12N 9/78; C12N 15/86

(52) U.S. Cl. CPC C12N 15/11; A61K 48/0066; A61P 25/28; C07K 14/47; C12N 9/78; C12N 15/86; C12Y 305/04004; C12N 2310/122; C12N 2310/333; C12N 2310/334; C12N 2310/336; C12N 2310/531; C12N 2310/533; C12N 2750/14143; C12N 2750/14171

(57) ABSTRACT

Disclosed herein are compositions that comprise engineered polynucleotides, pharmaceutical compositions comprising the same, methods of making the same, and methods of treatment comprising the compositions that comprise the engineered polynucleotides.

### CROSS-REFERENCE

This application is a continuation application of U.S. application Ser. No. 17/781,877, filed Jun. 2, 2022, which is a national phase application of PCT/US2020/062756, filed Dec. 1, 2020, which claims the benefit of U.S. Provisional Application No. 63/112,286, filed Nov. 11, 2020, U.S. Provisional Application No. 63/030,165, filed May 26, 2020, U.S. Provisional Application No. 63/022,727, filed May 11, 2020, U.S. Provisional Application No. 62/942,693, filed Dec. 2, 2019, U.S. Provisional Application No. 62/942,667, filed Dec. 2, 2019, and U.S. Provisional Application No. 62/942,683, filed Dec. 2, 2019, the disclosures of which are incorporated herein by reference in their entirety.

### SEQUENCE LISTING

The instant application contains a Sequence Listing which has been submitted electronically in ST.26 (xml) format and is hereby incorporated by reference in its entirety. Said ST.26 (xml) copy, created on Aug. 24, 2023, is named 199235_712301_xml and is 478,127 bytes in size.

### SUMMARY

Disclosed herein, in some embodiments, are engineered polynucleotides. In an aspect, an engineered polynucleotide comprises an engineered polynucleotide comprising a targeting sequence that is at least partially complementary to a region of a target RNA, wherein the region of the target RNA: (a) comprises a sequence that at least partially encodes for an amyloid precursor protein (APP) polypeptide; (b) comprises a sequence that is proximal to (a); or (c) comprises (a) and (b); and wherein the engineered polynucleotide is configured to facilitate an editing of a base of a nucleotide of the target RNA by an RNA editing entity.

In some embodiments, the editing of the base of the nucleotide of the target RNA by the RNA editing entity facilitates an increase or a decrease of: (a) a processing; (b) a cleavage; or (c) (a) and (b), of the APP polypeptide by a secretase enzyme, relative to an APP polypeptide encoded by the target RNA without the editing. In some embodiments, the secretase enzyme comprises: an alpha secretase; a beta secretase; a gamma secretase; or a combination thereof. In some embodiments, the secretase enzyme comprises the beta secretase, and wherein the beta secretase comprises beta-site amyloid precursor protein cleaving enzyme 1 (BACE1), cathepsin B, or Meprin beta.

In some embodiments, the engineered polynucleotide, when associated with the target RNA, further comprises a structural feature which at least in part recruits the RNA editing entity. In some embodiments, the structural feature comprises a bulge, an internal loop, a hairpin, a mismatch, a wobble base pair, or any combination thereof. In some embodiments, the structural feature comprises the bulge. In some embodiments, the bulge comprises an asymmetric bulge. In some embodiments, the bulge comprises a symmetric bulge. In some embodiments, the bulge comprises from about 1 to about 4 nucleotides of the engineered polynucleotide and from about 0 to about 4 nucleotides of the target RNA. In some embodiments, the bulge comprises from about 0 to about 4 nucleotides of the engineered polynucleotide and from about 1 to about 4 nucleotides of the target RNA. In some embodiments, the bulge comprises 3 nucleotides of the engineered polynucleotide and 3 nucleotides of the target RNA. In some embodiments, the structural feature comprises the internal loop. In some embodiments, the internal loop comprises an asymmetric internal loop. In some embodiments, the internal loop comprises a symmetric internal loop. In some embodiments, the internal loop is formed by from about 5 to about 10 nucleotides of either the engineered polynucleotide or the target RNA. In some embodiments, the structural feature comprises the hairpin. In some embodiments, the hairpin comprises a double stranded RNA molecule, and wherein the hairpin does not comprise the targeting sequence. In some embodiments, wherein a stem loop of the hairpin is from about 3 to about 15 nucleotides in length. In some embodiments, the structural feature comprises the mismatch. In some embodiments, the mismatch comprises a base in the targeting sequence of the engineered polynucleotide opposite to and unpaired with the base of the nucleotide of the target RNA. In some embodiments, the mismatch comprises a guanine-guanine mismatch. In some embodiments, the mismatch comprises an adenosine-cytosine mismatch, and wherein the adenosine is in the target RNA and the cytosine is in the targeting sequence of the engineered polynucleotide. In some embodiments, the adenosine in the adenosine-cytosine mismatch is the base of the nucleotide in the target RNA edited by the RNA editing entity. In some embodiments, the structural feature comprises the wobble base pair. In some embodiments, the wobble base pair comprises a guanine paired with a uracil. In some embodiments, the structural feature comprises a structural motif, and wherein the structural motif comprises two bulges and an adenosine-cytosine mismatch.

In some embodiments, the engineered polynucleotide further comprises an RNA editing entity recruiting domain that is capable of recruiting the RNA editing entity. In some embodiments, the RNA editing entity comprises an adenosine deaminase acting on RNA (ADAR) polypeptide or biologically active fragment thereof. In some embodiments, the ADAR polypeptide or the biologically active fragment thereof comprises ADAR1, ADAR2, or a biologically active fragment of any of these. In some embodiments, the ADAR polypeptide or biologically active fragment thereof is synthetically overexpressed in a neuronal cell that comprises the target RNA. In some embodiments, the engineered polynucleotide does not comprise an RNA editing entity recruiting domain. In some embodiments, the nucleotide is comprised in a codon which encodes an amino acid in proximity to a cleavage site of the APP polypeptide, and wherein the amino acid is at position 670, 671, 672, 673, 682, 684, 687, 712, or 714 of the APP polypeptide comprising the polypeptide sequence of SEQ ID NO: 2. In some embodiments, the cleavage site is selected from the group consisting of: an alpha-secretase cleavage site, a beta-secretase cleavage site, a beta′-secretase cleavage site, a gamma-secretase cleavage site, and any combination thereof. In some embodiments, the target RNA encodes for an unmodified APP polypeptide that comprises at least one amino acid residue difference as compared to the modified APP polypeptide generated from the editing of the base of the nucleotide of the target RNA. In some embodiments, the at least one amino acid residue difference comprises K670E, K670R, K670G, M671V, D672G, E682G, H684R, K687R, K687E, or K687G of the APP polypeptide comprising the polypeptide sequence of SEQ ID NO: 2. In some embodiments, the at least one amino acid residue difference comprises K670G or M671V of the APP polypeptide comprising the polypeptide sequence of SEQ ID NO: 2.

In some embodiments, the composition further comprises a second engineered polynucleotide comprising a second targeting sequence that is at least partially complementary to a region of a second target RNA. In some embodiments, the region of the second target RNA: (a) at least partially encodes for a tau polypeptide or an alpha-synuclein (SNCA) polypeptide; (b) comprises a sequence that is proximal to (a); or (c) comprises (a) and (b). In some embodiments, the region of the second target RNA at least partially encodes for the tau polypeptide, and wherein the region comprises: SEQ ID NO: 16-SEQ ID NO: 27. In some embodiments, the composition disclosed therein, wherein the region of the second target RNA at least partially encodes for the SNCA polypeptide, and wherein the region comprises: SEQ ID NO: 36-SEQ ID NO: 44.

In some embodiments, the editing further comprises editing of at least a second base of a second nucleotide of the target RNA by the RNA editing entity. In some embodiments, the editing of the base of the nucleotide of the target RNA or the second target RNA by the RNA editing entity is sufficient to reduce, prevent, or eliminate formation of: β-amyloid, SNCA polypeptide, tau polypeptide; or an RNA encoding the β-amyloid, the SNCA polypeptide, or the tau polypeptide, as determined by an in vitro assay comprising: (a) contacting the engineered polynucleotide or the second engineered polynucleotide with the target RNA or the second target RNA, and (b) determining a modulation of: a processing; a cleavage; or a processing and a cleavage; of a modified APP polypeptide encoded by an edited target RNA; a modified tau polypeptide encoded by an edited second target RNA; or a modified SNCA polypeptide encoded by the edited second target RNA; as compared to a modulation of a processing; a cleavage; or a processing and cleavage of an unmodified APP polypeptide encoded by an unedited target RNA; an unmodified tau polypeptide encoded by an unedited second target RNA; or an unmodified SNCA polypeptide encoded by the unedited second target RNA.

In some embodiments, the editing of the base of the nucleotide of the target RNA and the second target RNA by the RNA editing entity is sufficient to reduce, prevent, or eliminate formation of the β-amyloid, and wherein the β-amyloid comprises: an Abeta40 fragment, an Abeta42 fragment, or the Abeta40 fragment and the Abeta 42 fragment. In some embodiments, the editing of the base of the nucleotide of the target RNA and the second target RNA by the RNA editing entity is sufficient to reduce the formation by about 1-fold, 3-fold, 5-fold, 10-fold, or 50-fold, as compared to an otherwise comparable cell lacking the contact with the composition. In some embodiments, the editing is sufficient to eliminate the β-amyloid peptide formation. In some embodiments, the editing is sufficient to increase an amount of secreted ectodomain APP alpha (sAPPa).

Disclosed herein, in some embodiments, are compositions. In an aspect, a composition comprises: (a) an engineered polynucleotide comprising a targeting sequence that is at least partially complementary to a region of a target RNA, wherein the region of the target RNA at least partially encodes for an amyloid precursor protein (APP) polypeptide; and (b) a second engineered polynucleotide comprising a second targeting sequence that is at least partially complementary to a region of a second target RNA, wherein the region of the second target RNA at least partially encodes for: a tau polypeptide or an alpha-synuclein (SNCA) polypeptide, and wherein the engineered polynucleotide and the second engineered polynucleotide are independently configured to facilitate an editing of a base of a nucleotide of the target RNA or the second target RNA by an RNA editing entity.

In some embodiments, the region of the second target RNA at least partially encodes for the tau polypeptide, and wherein the region comprises: SEQ ID NO: 16-SEQ ID NO: 27. In some embodiments, the region of the second target RNA at least partially encodes for the SNCA polypeptide, and wherein the region comprises: SEQ ID NO: 36-SEQ ID NO: 44.

In some embodiments, the editing of the base of the nucleotide of the target RNA or the second target RNA by the RNA editing entity is sufficient to reduce or eliminate formation of: β-amyloid, SNCA polypeptide, tau polypeptide; or an RNA encoding the β-amyloid, the SNCA polypeptide, or the tau polypeptide, as determined by an in vitro assay comprising: (a) contacting the engineered polynucleotide with the target RNA or the second engineered polynucleotide with the second target RNA, and (b) determining a modulation of: a processing; a cleavage; or a processing and a cleavage of a modified APP polypeptide encoded by an edited target RNA; a modified tau polypeptide encoded by an edited second target RNA; or a modified SNCA polypeptide encoded by the edited second target RNA; as compared to a modulation of: a processing; a cleavage; or a processing and cleavage of an unmodified APP polypeptide encoded by an unedited target RNA; an unmodified tau polypeptide encoded by an unedited second target RNA; or an unmodified SNCA polypeptide encoded by the unedited second target RNA.

In some embodiments, the editing of the base of the nucleotide of the target RNA and the second target RNA by the RNA editing entity is sufficient to reduce the formation by about 1-fold, 3-fold, 5-fold, 10-fold, or 50-fold, as compared to an otherwise comparable cell lacking the contact with the composition. In some embodiments, the modulation is determined by measuring a level of: a) the modified APP polypeptide, the modified Tau polypeptide, the modified SNCA polypeptide, or a combination of any of these; b) a mRNA transcript encoding the modified APP polypeptide, a mRNA transcript encoding the modified Tau polypeptide, a mRNA transcript encoding the modified SNCA polypeptide, or a combination of any of these; c) phosphorylation of the modified APP polypeptide, phosphorylation of the modified Tau polypeptide, phosphorylation of the modified SNCA polypeptide, or a combination of any of these; d) aggregation of the modified APP polypeptide, aggregation of the modified Tau polypeptide, aggregation of the modified SNCA polypeptide, or a combination of any of these; or e) a combination of any of these.

Disclosed herein, in some embodiments, are vectors. In an aspect, a vector comprises: (a) a polynucleotide sequence that encodes the engineered polynucleotide described herein and thereof; (b) a second engineered polynucleotide; or (c) a combination of any of these. In some embodiments, the third engineered polynucleotide comprises an siRNA, an shRNA, a miRNA, a piRNA, an antisense oligonucleotide; or does not comprise at least one of these. In some embodiments, the AAV vector is of a serotype selected from the group comprising: AAV2, AAV5, AAV6, AAV8, AAV9, a portion thereof, a fusion product thereof, and any combination thereof. In some embodiments, the AAV vector comprises rep and inverted terminal repeats (ITR) sequences from AAV2 and a cap sequence from AAV5. In some embodiments, the AAV vector comprises an ITR sequence that is an ITR with a mutated terminal resolution site (TRS).

Disclosed herein, in some embodiments, are pharmaceutical composition in unit dose forms. In an aspect, a pharmaceutical composition in unit dose form comprises the engineered polynucleotide described herein and thereof or the vector described herein and thereof.

Disclosed herein, in some embodiments, are methods of treating or preventing a disease or condition in a subject in need thereof. In an aspect, a method of treating or preventing a disease or condition in a subject in need thereof comprises administering to the subject: (a) the vector described herein and thereof; (b) the pharmaceutical composition described herein and thereof; or (c) (a) and (b), wherein after the administering, the subject comprises: (a) at least a 1-fold reduced formation of β-amyloid as compared to an otherwise comparable subject lacking the administering, as measured by: a brain scan, a blood test, or both; (b) or at least a 1-fold increase in secreted ectodomain APP alpha (sAPPa), as compared to an otherwise comparable subject lacking the administering, as determined by an in vitro assay comprising: contacting the engineered polynucleotide with the target RNA and determining a level of the sAPPa by Western Blot.

In some embodiments, the β-amyloid comprises at least one of: an Abeta 40 fragment, an Abeta42 fragment, or both.

Disclosed herein, in some embodiments, are methods of treating or preventing a disease or condition in a subject in need thereof. In an aspect, a method of treating or preventing a disease or condition in a subject in need thereof comprises: administering to the subject a composition that comprises an engineered polynucleotide or a vector that encodes the engineered polynucleotide, wherein the engineered polynucleotide comprises a targeting sequence that is at least partially complementary to a region of a target RNA, wherein the region of the target RNA: (a). comprises a sequence that at least partially encodes for an amyloid precursor protein (APP) polypeptide; (b) comprises a sequence that is proximal to (a); or (c). comprises (a) and (b), wherein the engineered polynucleotide is configured to facilitate an editing of a base of a nucleotide of the target RNA by an RNA editing entity, whereby the edited target RNA encodes for a modified APP polypeptide that has reduced susceptibility to cleavage by a beta secretase, as compared to an unmodified APP polypeptide encoded by an otherwise comparable unedited target RNA, wherein the reduced susceptibility to cleavage of the modified APP polypeptide results in reduced β-amyloid formation, as determined by: i. an in vitro assay comprising contacting the engineered polynucleotide with the target RNA and determining cleavage of the modified APP polypeptide encoded by the edited target RNA by the beta secretase as compared to cleavage of the unmodified APP polypeptide encoded by the unedited target RNA; ii. an in vivo diagnostic after the administering; iii. an in vitro assay comprising a blood test after the administering; iv. histology of a brain tissue of the subject after the administering; or v. any combination thereof.

In some embodiments, the beta secretase comprises: beta-site amyloid precursor protein cleaving enzyme 1 (BACE1), cathepsin B, or Meprin beta. In some embodiments, the engineered polynucleotide, when associated with the target RNA, further comprises a structural feature which at least in part recruits the RNA editing entity. In some embodiments, the engineered polynucleotide further comprises an RNA editing entity recruiting domain. In some embodiments, the disease or condition comprises a neurodegenerative disease or condition. In some embodiments, the neurodegenerative disease or condition comprises Alzheimer's disease, Parkinson's disease, a dementia, Lewy Body Dementia, a progressive supranuclear palsy, a frontotemporal lobar degeneration, a corticobasal degeneration, or any combination thereof.

In some embodiments, the method further comprises a second administering. In some embodiments, the administering, the second administering, or both, are independently repeated at least once a month. In some embodiments, the administering, the second administering, or both, are independently performed by a: parenteral route, oral route, respiratory route, intraduodenal route, rectal route, or a combination thereof. In some embodiments, the in vivo diagnostic comprises: a positron emission tomography scan, a computerized tomography scan, magnetic resonance imaging, spinal tap, or a combination thereof.

In some embodiments, the modified APP polypeptide has increased susceptibility to cleavage by an alpha secretase, as compared to an unmodified APP polypeptide encoded by an unedited target RNA polypeptide. In some embodiments, the cleavage of the modified APP polypeptide by the alpha secretase results in an increased amount of secreted ectodomain APP alpha (sAPPa) in the subject as compared to an otherwise comparable subject lacking the administering. In some embodiments, the reduced β-amyloid formation comprises at least about a 1-fold, 3-fold, 5-fold, 10-fold, or 50-fold reduction as compared to an otherwise comparable subject lacking the administering.

In some embodiments, the vector comprises an AAV vector of a serotype selected from the group comprising: AAV2, AAV5, AAV6, AAV8, AAV9, a portion thereof, a fusion product thereof, or any combination thereof. In some embodiments, the composition further comprises: (a) a second engineered polynucleotide; (b) a second vector encoding the second engineered polynucleotide; (c) the vector further encoding the second engineered polynucleotide; or (d) any combination thereof, wherein the second engineered polynucleotide comprises a second targeting sequence that is at least partially complementary to a region of a second target RNA. In some embodiments, the region of the second target RNA: (a) at least partially encodes for a tau polypeptide or an alpha-synuclein (SNCA) polypeptide; (b) comprises a sequence that is proximal to (a); or (c) comprises (a) and (b). In some embodiments, the editing of the base of the nucleotide of the target RNA or the second target RNA by the RNA editing entity is sufficient to reduce or eliminate formation of: β-amyloid, SNCA polypeptide, tau polypeptide; or an RNA encoding the β-amyloid, the SNCA polypeptide, or the tau polypeptide, as determined by an in vitro assay comprising: (a) contacting the engineered polynucleotide with the target RNA or the second engineered polynucleotide with the second target RNA, and (b) determining a modulation of: a processing; a cleavage; or a processing and a cleavage of a modified APP polypeptide encoded by an edited target RNA; a modified tau polypeptide encoded by an edited second target RNA; or a modified SNCA polypeptide encoded by the edited second target RNA; as compared to a modulation of: a processing; a cleavage; or a processing and cleavage of an unmodified APP polypeptide encoded by an unedited target RNA; an unmodified tau polypeptide encoded by an unedited second target RNA; or an unmodified SNCA polypeptide encoded by the unedited second target RNA.

In some embodiments, wherein when an ex vivo population of neuronal cells is contacted with the composition, at least 5% of the neuronal cells in the population are edited after the contacting, as measured by Sanger sequencing. In some embodiments, wherein at least 10%, 15%, 20%, 30%, 40%, or 50% of the neuronal cells in the population are edited. In some embodiments, the editing further comprises editing of at least a second base of a second nucleotide of the target RNA by the RNA editing entity. In some embodiments, the subject is diagnosed with the disease or condition. In some embodiments, the method further comprises a second administering of an additional therapeutic agent. In some embodiments, the administering and the second administering are consecutive. In some embodiments, the administering and the second administering are concurrent.

Disclosed herein, in some embodiments, are engineered polynucleotides. In an aspect, an engineered polynucleotide comprises a sequence that comprises at least 90%, 95%, 97%, or 99% sequence identity with at least a portion of a sequence selected from: SEQ ID NO: 52-SEQ ID NO: 52, SEQ ID NO: 71-SEQ ID NO: 148, and SEQ ID NO: 159-SEQ ID NO: 167.

Disclosed herein, in some embodiments, are engineered polynucleotides. In an aspect, an engineered polynucleotide comprises a targeting sequence capable of at least partially binding to a sequence that comprises at least 90%, 95%, 97%, or 99% sequence identity with a portion of a sequence selected from: SEQ ID NO: 150-SEQ ID NO: 158 as determined by BLAST.

Disclosed herein, in some embodiments, are engineered polynucleotides. In an aspect, an engineered polynucleotide comprises a targeting sequence that is at least partially complementary to a region of a target RNA, wherein the region of the target RNA: (a) at least partially encodes for: an amyloid precursor protein (APP) polypeptide, an alpha-synuclein (SNCA) polypeptide, or a Tau polypeptide; (b) comprises a sequence that is proximal to (a); or (c) comprises (a) and (b); wherein the engineered polynucleotide is configured to: facilitate an editing of a base of a nucleotide of a polynucleotide in the region of the target RNA by an RNA editing entity; facilitate a modulation of the expression of the APP polypeptide, the SNCA polypeptide, or the Tau polypeptide; or a combination thereof.

In some embodiments, the facilitating the editing of the base of the nucleotide of the polynucleotide in the region of the target RNA by the RNA editing entity, the engineered polynucleotide is configured to facilitate modulation of processing and/or cleavage of the target RNA by a secretase enzyme. In some embodiments, the target RNA is the APP polypeptide. In some embodiments, the region of the target RNA is cleaved by a secretase enzyme. In some embodiments, the secretase is: a beta secretase; a γ-secretase; or a beta secretase and a γ-secretase. In some embodiments, the beta secretase, and wherein the beta secretase comprises beta-site amyloid precursor protein cleaving enzyme 1, cathepsin B, or Meprin beta. In some embodiments, for the engineered polynucleotide comprising (b), the sequence that is proximal to the region of the target RNA at least partially encoding the APP polypeptide, the SNCA polypeptide, or the Tau polypeptide comprises at least a portion of a three prime untranslated region (3′ UTR). In some embodiments, for the engineered polynucleotide comprising (b), the sequence that is proximal to the region of the target RNA at least partially encoding the APP polypeptide, the SNCA polypeptide, or the Tau polypeptide comprises at least a portion of a five prime untranslated region (5′ UTR). In some embodiments, the editing of a base of the 5′UTR results in at least partially regulating gene translation of the APP polypeptide, the SNCA polypeptide, or the Tau polypeptide. In some embodiments, the editing of the base of the nucleotide of the polynucleotide of the region of the 5′UTR results in facilitating regulating mRNA translation of the APP. In some embodiments, for the engineered polynucleotide comprising (b), the sequence that is proximal to the region of the target RNA at least partially encoding the APP polypeptide, the SNCA polypeptide, or the Tau polypeptide comprises at least a portion of: a poly(A) tail, microRNA response element (MRE), AU-rich element (ARE), or any combination thereof. In some embodiments, the region of the target RNA at least partially encodes for the APP polypeptide. In some embodiments, the region of the target RNA at least partially encodes for the SNCA polypeptide. In some embodiments, the region of the target RNA at least partially encodes for the Tau polypeptide. In some embodiments, the engineered polynucleotide is configured to facilitate the cleavage of the target RNA by the beta-site amyloid precursor protein cleaving enzyme 1. In some embodiments, the engineered polynucleotide is configured to facilitate the editing of the base of the nucleotide of the polynucleotide of the region of the target RNA by the RNA editing entity. In some embodiments, the targeting sequence that is at least partially complementary to the region of the target RNA comprises at least one nucleotide that is not complementary to a nucleotide in the region of the target RNA. In some embodiments, the at least one nucleotide that is not complementary is an adenosine (A), and wherein the A is comprised in an A/C mismatch. In some embodiments, the target RNA is selected from the group comprising: a mRNA, a tRNA, a lncRNA, a lincRNA, a miRNA, a rRNA, a snRNA, a microRNA, a siRNA, a piRNA, a snoRNA, a snRNA, a exRNA, a scaRNA, a YRNA, and a hnRNA. In some embodiments, the target RNA is the mRNA. In some embodiments, the region of the target RNA comprises a mutation as compared to an otherwise comparable region encoding a wildtype APP polypeptide, a wildtype SNCA polypeptide, or a wildtype Tau polypeptide. In some embodiments, the mutation comprises a polymorphism. In some embodiments, the targeting sequence is about: 40, 60, 80, 100, or 120 nucleotides in length. In some embodiments, the targeting sequence is about 100 nucleotides in length.

In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length.

The engineered polynucleotide disclosed herein, wherein the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequences comprises SEQ ID NO: 1. In some embodiments, the RNA editing entity comprises an adenosine deaminase acting on RNA (ADAR) polypeptide or biologically active fragment thereof or adenosine deaminase acting on tRNA (ADAT) polypeptide or biologically active fragment thereof. In some embodiments, the ADAR polypeptide or biologically active fragment thereof, which comprises ADAR1 or ADAR2. In some embodiments, the engineered polynucleotide lacks a recruiting domain.

In some embodiments, the engineered polynucleotide further comprises a structural feature which at least in part recruits an RNA editing entity. In some embodiments, the structural feature comprises: a bulge, a hairpin, an internal loop, a structured motif, and any combination thereof. In some embodiments, the structural feature comprises the bulge. In some embodiments, the bulge is an asymmetric bulge. In some embodiments, the bulge is a symmetric bulge. In some embodiments, the bulge is from 1-29 nucleotides in length. In some embodiments, the structural feature comprises the hairpin. In some embodiments, the structural feature comprises the internal loop. In some embodiments, the internal loop is an asymmetric loop. In some embodiments, the internal loop is a symmetric loop. In some embodiments, the structural feature comprises the structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop.

In some embodiments, the engineered polynucleotide comprises a backbone that comprises a plurality of sugar and phosphate moieties covalently linked together, and wherein the backbone comprises a 5′ reducing hydroxyl, a 3′ reducing hydroxyl, or both. In some embodiments, each of the 5′ reducing hydroxyl in the backbone is linked to each of the 3′ reducing hydroxyl via a phosphodiester bond. In some embodiments, the engineered polynucleotide comprises a backbone that comprises a plurality of sugar and phosphate moieties covalently linked together, and wherein the backbone lacks a 5′ reducing hydroxyl, a 3′ reducing hydroxyl, or both. In some embodiments, the engineered polynucleotide associates with the region of the target RNA, the association comprises hybridized polynucleotide strands. In some embodiments, the hybridized polynucleotide strands at least in part form a duplex. In some embodiments, the engineered polynucleotide further comprises a chemical modification. In some embodiments, the engineered polynucleotide comprises RNA, DNA, or both. In some embodiments, the engineered polynucleotide comprises the RNA.

Disclosed herein, in some embodiments, are engineered polynucleotides. In an aspect, an engineered polynucleotide is configured to facilitate an editing of a base of a nucleotide of a polynucleotide of a region of a target RNA at least partially encoding an amyloid precursor protein (APP), wherein an RNA editing entity, in association with the engineered polynucleotide and the target RNA, edits the base of the nucleotide of the polynucleotide of the region of the target RNA, wherein the editing results in generation of an edited target RNA at least partially encoding a modified amyloid precursor protein (APP).

In some embodiments, the RNA editing entity comprises a secretase enzyme. In some embodiments, the secretase enzyme is beta secretase; a γ-secretase; or a beta secretase and a γ-secretase. In some embodiments, the secretase enzyme is the beta secretase, and wherein the beta secretase is selected from the group consisting of: beta-site amyloid precursor protein cleaving enzyme 1, cathepsin B, and Meprin beta.

Disclosed herein, in some embodiments, are engineered polynucleotides. In an aspect, an engineered polynucleotide is configured to facilitate, by an RNA editing entity, an editing of a base of a nucleotide of a polynucleotide of a region of a target RNA at least partially encoding an amyloid precursor protein (APP), wherein the editing results in generation of an edited target RNA that comprises at least one amino acid substitution compared to an otherwise comparable unedited target RNA, wherein the edited target RNA encodes an APP with an altered susceptibility to a beta secretase cleavage compared to the otherwise comparable APP encoded by the otherwise comparable unedited target RNA; and wherein a cell expressing an APP polypeptide generated from the edited target RNA has substantially no decrease in beta secretase activity on an endogenous substrate of beta secretase compared to a corresponding cell expressing an APP polypeptide generated from the unedited target RNA, as determined by an in vitro assay comprising a measurement of a metabolite indicative of cleavage of the endogenous substrate by beta-site amyloid precursor protein cleaving enzyme 1 (BACE1), and wherein the endogenous substrate comprises amyloid-like protein 1 (APLP1), amyloid-like protein 2 (APLP2), Contactin 2, Jagged 1, neural cell adhesion molecule L1 (CHL1), Neurexin 1α, Neurexin 3β, neuregulin 1 (NRG1), seizure related protein 6 (SEZ6), seizure related protein 6 precursor protein (SEZ6L), a β (β1-4) Auxiliary subunit of the voltage-gated sodium ion channel (VGSC) subtype Nav1, VGSC Accessory Subunits KCNE1 or KCNE2, a functional portion of any of these, or any combination of thereof.

In some embodiments, the beta secretase comprises BACE1, cathepsin B, or Meprin beta. In some embodiments, the endogenous substrate comprises the NRG1, the SEZ6, or the CHL1.

Disclosed herein, in some embodiments, are engineered polynucleotides. In an aspect, an engineered polynucleotide is configured to facilitate, by an RNA editing entity, an editing of a base of a nucleotide of a polynucleotide of a region of a target RNA at least partially encoding an amyloid precursor protein (APP), wherein the editing results in generation of a modified APP encoded by an edited target RNA that comprises at least one amino acid substitution compared to an otherwise comparable unmodified APP encoded by an comparable unedited target RNA, and wherein the modified APP polypeptide generated from the edited target RNA: (i) produces a lower amount of Abeta40, Abeta42, or both when expressed in a cell as compared to an APP polypeptide generated from the unedited target RNA as measured by an Abeta40 or Abeta42 enzyme linked immunosorbent assay (ELISA); (ii) produces an increased amount of secreted ectodomain APP alpha (sAPPa) when expressed in a cell as compared to the sAPPa generated from the unedited target RNA as measured by an sAPPa ELISA; or (iii) any combination of (i) and (ii).

Disclosed herein, in some embodiments, are vectors. In an aspect, a vector comprises: (a) the engineered polynucleotides herein and thereof (b) a polynucleotide encoding the engineered polynucleotides herein and thereof; or (c) (a) and (b).

In some embodiments, the vector further comprises a second engineered polynucleotide or a second polynucleotide encoding the second engineered polynucleotide. In some embodiments, the engineered polynucleotide and the second engineered polynucleotide are the same. In some embodiments, the engineered polynucleotide and the second engineered polynucleotide are different. In some embodiments, the second engineered polynucleotide comprises a second targeting sequence that at least partially hybridizes to a region of a second target RNA. In some embodiments, the second engineered polynucleotide comprises an siRNA, an shRNA, an miRNA, a piRNA, an anti-sense oligonucleotide; or does not comprise at least one of these. In some embodiments, the engineered polynucleotide and the second engineered polynucleotide are contiguous with each other. In some embodiments, the polynucleotide of the vector independently encodes: the engineered polynucleotide and the second engineered polynucleotide, are operatively linked to a same promoter sequence. In some embodiments, the engineered polynucleotide and the second engineered polynucleotide not contiguous with each other.

In some embodiments, the engineered polynucleotide comprises a targeting sequence that is at least partially complementary to a region of the APP target RNA, and wherein the second engineered polynucleotide comprises a targeting sequence that is at least partially complementary to a region of the SNCA or the Tau target RNA. In some embodiments, the engineered polynucleotide comprises a targeting sequence that is at least partially complementary to a region of the APP target RNA, and wherein the second engineered polynucleotide comprises the siRNA, the shRNA, the miRNA, the piRNA, or the antisense oligonucleotide that targets the SNCA polypeptide or the Tau polypeptide.

In some embodiments, the vector is a viral vector. In some embodiments, the viral vector is an AAV vector, and wherein the AAV vector is of a serotype selected from the group comprising: AAV2, AAV5, AAV6, AAV8, AAV9, a portion thereof, a fusion product thereof, and any combination thereof. In some embodiments, the AAV vector comprises rep and ITR sequences from AAV2 and a cap sequence from AAV5. In some embodiments, the AAV vector comprises an ITR sequence that is a self-complementary ITR. In some embodiments, the AAV vector that encodes for the engineered polynucleotide is self-complementary.

Disclosed herein, in some embodiments, are pharmaceutical compositions in unit dose form. In an aspect, a pharmaceutical composition in unit dose dorm comprises the engineered polynucleotides and vectors herein and thereof. In some embodiments, the pharmaceutical composition in unit dose further comprises a pharmaceutically acceptable: excipient, carrier, or diluent.

Disclosed herein, in some embodiments, are methods of making compositions. In an aspect, a method of making a pharmaceutical composition comprising admixing the engineered polynucleotides herein and thereof with a pharmaceutically acceptable excipient, diluent, or carrier.

Disclosed herein, in some embodiments, are isolated cells. In an aspect, an isolated cell comprises the engineered polynucleotides herein or thereof, the vectors herein or thereof, or both.

Disclosed herein, in some embodiments, are kits. In an aspect, a kit comprises the engineered polynucleotides herein and thereof, the vectors herein and thereof, or both in a container. In some embodiments, the kit comprises inserting the engineered polynucleotide disclosed therein into a container.

Disclosed herein, in some embodiments, are methods of treating or preventing a disease or condition in a subject in need thereof. In an aspect, a method of treating or preventing a disease or condition in a subject in need thereof comprises administering to a subject in need thereof: (a) the vectors herein and thereof; (b) the pharmaceutical compositions herein and thereof; or (c) (a) and (b).

Disclosed herein, in some embodiments, are methods of treating or preventing a disease or condition. In an aspect, a method of treating or preventing a disease or condition comprises administering a therapeutic to a subject in need thereof, wherein the therapeutic facilitates, by an RNA editing entity, an editing of a base of a nucleotide of a polynucleotide of a region of a target RNA that at least partially encodes for an amyloid precursor protein (APP), thereby generating an edited RNA that at least partially encodes for a beta secretase-resistant APP as compared to an otherwise comparable APP encoded by an otherwise comparable RNA lacking the edit as determined by in vitro assay comprising contacting the beta secretase-resistant APP and the otherwise comparable APP with: a) a beta secretase; b) a γ-secretase; c) or a beta secretase and a γ-secretase.

In some embodiments, the beta secretase comprises beta-site amyloid precursor protein cleaving enzyme 1, cathepsin B, or Meprin beta. In some embodiments, the therapeutic comprises a vector comprising or encoding an engineered polynucleotide that comprises a targeting sequence that at least partially hybridizes to a region of the target RNA. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain comprises a glutamate ionotropic receptor AMPA type subunit 2 (GluR2) sequence. In some embodiments, the RNA editing entity comprises an adenosine deaminase acting on RNA (ADAR) polypeptide or biologically active fragment thereof or adenosine deaminase acting on tRNA (ADAT) polypeptide or biologically active fragment thereof. In some embodiments, the RNA editing entity comprises the ADAR polypeptide or biologically active fragment thereof, and wherein the ADAR comprises ADAR1 or ADAR2. In some embodiments, the engineered polynucleotide lacks a RNA editing entity recruiting sequence.

In some embodiments, the engineered polynucleotide further comprises a structural feature. In some embodiments, the structural feature comprises: a bulge, a hairpin, an internal loop, a structured motif, and any combination thereof. In some embodiments, the structural feature comprises the bulge. In some embodiments, the bulge is an asymmetric bulge. In some embodiments, the bulge is a symmetric bulge. In some embodiments, the bulge is from 1-29 nucleotides in length. In some embodiments, the structural feature comprises the hairpin. In some embodiments, the structural feature comprises the internal loop. In some embodiments, the internal loop is asymmetric. In some embodiments, the internal loop is asymmetric. In some embodiments, the structural feature comprises the structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop.

In some embodiments, the engineered polynucleotide comprises a backbone that comprises a plurality of sugar and phosphate moieties covalently linked together, and wherein the backbone comprises a 5′ reducing hydroxyl, a 3′ reducing hydroxyl, or both. In some embodiments, each of the 5′ reducing hydroxyl in the backbone is linked to each of the 3′ reducing hydroxyl via a phosphodiester bond. In some embodiments, the engineered polynucleotide comprises a backbone that comprises a plurality of sugar and phosphate moieties covalently linked together, and wherein the backbone lacks a 5′ reducing hydroxyl, a 3′ reducing hydroxyl, or both.

In some embodiments, the beta secretase-resistant APP has reduced susceptibility to cleavage at a position cleavable by a beta secretase as compared to the otherwise comparable APP produced from the otherwise comparable RNA lacking the edit. In some embodiments, the beta secretase comprises BACE1, cathepsin B, or Meprin beta. In some embodiments, the nucleotide is comprised in a codon which encodes an amino acid in proximity to a cleavage site of the APP. In some embodiments, the cleavage site at the APP is selected from the group consisting of: an α-secretase cleavage site, a β-secretase cleavage site, a B′-secretase cleavage site, a γ-secretase cleavage site, and any combination thereof. In some embodiments, the amino acid is at position 669, 670, 671, 672, 673, 682, 683, 684, 687, 688, 711, 712, 713, or 714 of the APP of SEQ ID NO: 2. In some embodiments, the BACE protease-resistant APP comprises at least one amino acid residue difference as compared to the otherwise comparable APP produced from the otherwise comparable RNA lacking the edit. In some embodiments, the one amino acid residue difference comprises an amino acid substitution that results in a change in charge, hydrophobicity, or polarity of the amino acid, or any combination thereof. In some embodiments, the difference in the amino acid comprises a conservative substitution. In some embodiments, the difference in the amino acid comprises a charge neutral substitution. In some embodiments, the amino acid residue comprises a K to E change, a K to R change, a K to G change, an M to V change, a D to G change, an E to G change, an H to R change, or any combination thereof. In some embodiments, the difference in the amino acid residue comprises K670E, K670R, K670G, M671V, D672G, E682G, H684R, K687R, K687E, or K687G of the amyloid precursor protein of SEQ ID NO: 2. In some embodiments, the change in the one amino acid comprises K670G or M671V of the amyloid precursor protein of SEQ ID NO: 2.

In some embodiments, the target RNA is selected from the group comprising: an mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is the mRNA.

In some embodiments, the therapeutic directly facilitates the edit. In some embodiments, the therapeutic indirectly facilitates the edit. In some embodiments, the disease or condition comprises a neurodegenerative disease or condition. In some embodiments, the neurodegenerative condition comprises Alzheimer's disease, Parkinson's disease, dementia, Lewy Body Dementia, progressive supranuclear palsy, frontotemporal lobar degeneration, corticobasal degeneration, or any combination thereof. In some embodiments, the condition comprises traumatic brain injury, Down's syndrome, cancer, Fragile X Syndrome, autism, amyotrophic lateral sclerosis, multiple sclerosis, Lesch-Nyhan disease, metabolic disorder, or any combination thereof. In some embodiments, the edited RNA or the BACE protease-resistant APP is generated in at least 5%, 8%, 10%, 15%, 20%, 30%, 40%, or 50% of the subjects administered the therapeutic in a clinical trial. In some embodiments, the method further comprises a second administering of an additional therapeutic agent. In some embodiments, the administering and the second administering are consecutive. In some embodiments, the administering and the second administering are concurrent. In some embodiments, the administering or the second administering or both are independently repeated at least once a week. In some embodiments, the administering or the second administering or both are independently performed by parenteral route of administration. In some embodiments, the administering or the second administering or both are independently performed by parenchymal injection, intra-thecal injection, intra-ventricular injection, intra-cisternal injection, intravenous injection, or intranasal administration or any combination thereof.

### BRIEF DESCRIPTION OF THE DRAWINGS

FIG. 1 shows a Venn diagram of diseases caused by different pathogenic proteins. Abeta plaques, alpha-synuclein Lewy bodies and Tau NFT pathologies in combination can confer a worse prognosis for a subject. Alzheimer's disease can share protein pathologies with other neurodegenerative diseases. For example, Alzheimer's disease (AD) can share protein pathologies with one or more of: CBD: Corticobasal degeneration; DLB: Dementia with Lewy bodies; FTDP-17T: Fronto-temporal dementia with Parkinsonism linked to Tau mutations on chromosome 17; LBVAD: Lewy body variant of Alzheimer's Disease; PD: Parkinson's Disease; PDD: Parkinson's Disease with dementia; PiD: Pick's Disease; or PSP: Progressive supranuclear palsy.

FIG. 2A shows an exemplary schematic of the processing of APP. It also shows the beta and gamma secretase cleavage sites on APP that can lead to Abeta fragment formation. Further, the pathogenic mutations that have been implicated in Alzheimer's diseases in different populations are shown. Autosomal dominant genetic mutations around β- and γ-secretase cleavage sites can cause either elevated levels of total Abeta metabolite production or can cause a specific increase in Abeta 42 peptides, which can be more hydrophobic, which may be associated with early onset Alzheimer's disease risk (both increased and decreased). These data have been used to support development and clinical testing of β- and γ-secretase inhibitors. Examples of such mutations are further described. For example, the A673V mutation can shift APP processing toward the amyloidogenic pathway, with increased production of Abeta peptides, and markedly can enhance the aggregation and fibrillogenic properties of both Abeta 40 and Abeta 42. The A673T mutation lies adjacent to the β-secretase cleavage site in the APP gene and can result in an about 40% reduction in the formation of Abeta peptides in vitro. This variant was found to be significantly more common in the aged control group than in AD cases, suggesting that A673T reduces the risk for AD. Genetics and genomic studies of APP have identified 52 pathogenic mutations in APP that can lead to Abeta deposition in the brain parenchyma and in cerebral blood vessels. Studies of these families have shown that over-expression of the normal APP sequence (trisomy 21 or APP duplication) or mutations that lead to elevated total Abeta, elevated Abeta 42, or increased Abeta aggregation can lead to dementia or AD neuropathology. Figure discloses SEQ ID NO: 197. FIG. 2B shows an exemplary schematic showing possible sites for mRNA base editing of APP. Using mRNA base editing, the compositions as described herein can specifically alter amino acid residues that can be critical for BACE1-mediated proteolysis, thereby reducing or eliminating formation of Abeta fragments associated with increased Alzheimer's disease risk while preserving both BACE1 function and normal APP levels. Figure discloses SEQ ID NOS 197-198, respectively, in order of appearance.

FIG. 2C shows a cartoon schematic of the APP processing in vivo. APP protein, 201, has a N-terminal ectodomains and a shorter C-terminus that contains a crucial Tyrosine-Glutamic Acid-Asparagine-Proline-Threonine-Tyrosine (YENPTY) (SEQ ID NO: 193) protein-sorting domain to which the adaptor proteins X11 and Fe65 bind. The fragment, 202, containing the Abeta peptide, from 672^{nd} to 727^{th} amino acid of SEQ ID NO: 2, is magnified in 203. The alpha, beta, beta′, and gamma cleavage sites are listed on the amino acid sequence in 203. Figure also discloses SEQ ID NO: 199.

FIG. 2D and FIG. 2E depict cartoon schematic showing normal and pathogenic processing of APP. FIG. 2D shows that during the normal and nonamyloidogenic processing of APP, cleavages with alpha secretase followed by gamma secretase will generate soluble ectodomains alpha sAPP alpha, an intracellular C-terminal fragment AICD, and p3 fragments. FIG. 2E shows that during pathogenic and amyloidogenic processing of APP, cleavages involving BACE1 followed by gamma secretase will generate a soluble ectodomain sAPP beta AICD, and pathogenic Abeta fragments.

FIG. 3 shows APP, the generation of Abeta fragments, and the effects of the A673T and A673V mutations in APP processing. The amino acid sequence of a portion of APP is shown. The Abeta 40 length and Abeta 42 sequences are also shown. Additionally, the location of the A673T mutation is shown, a mutation that can be protective (reduced Abeta 1-Abeta 42 levels, improved cognition with age) and can suggest reduced BACE1 cleavage. The location of the A673V mutation is shown, a mutation that can confer increased risk (increased Abeta 1-Abeta 42 levels, impaired cognition) and can suggest increased BACE1 cleavage. The amino acid consensus sequence of full length APP is shown in SEQ ID NO: 2. Figure discloses SEQ ID NO: 200.

FIG. 4 shows an exemplary method for diagnosing a disease, condition, or for monitoring progression thereof. In the method, a sample 402 containing genetic material can be obtained from a subject 401, such as a human subject presumed to have a mutation in APP. A sample 402 can be subjected to one or more methods described herein. In some cases, a method can comprise performing sequencing (such as high-throughput sequencing), genotyping, hybridization, amplification, labeling, or any combination thereof (403). One or more results from a method can be input into a processing module 406. In 406, one or more input parameters such as a sample identification, subject identification, sample type, a reference, or other information can be input into a processor 404. One or more metrics from an assay can be input into the processor 404 such that the processor can produce a result, such as a diagnosis of a neurodegenerative condition, a risk of developing a neurodegenerative condition, an identification of a treatment for a neurodegenerative condition, a responsiveness to a treatment for a neurodegenerative condition, or any combination thereof. A processor can send a result, an input parameter, a metric, a reference, or any combination thereof to a display 405, such as a visual display or graphical user interface. The processor 404 can (i) send a result, an input parameter, a metric, or any combination thereof to a server 407, (ii) receive a result, an input parameter, a metric, or any combination thereof from the server 407, (iii) or a combination thereof.

FIG. 5 shows an APP amino acid schematic carton denoting the non-limiting examples of target mutation sites in the APP protein sequence to modulate cleavage by proteases and lower production of Abeta fragments. APP contains numerous cleavage sites which are cleaved by endogenous proteases, such as beta-secretase 1 (BACE1) and alpha secretases (e.g., ADAM10). Mutations near the β-site, β′-site, and α-site as indicated in FIG. 5 were identified as amenable to editing by ADAR and were selected for further analysis. The 15 target mutations are shown in TABLE 1 below, along with the nearest cleavage site. Figure discloses SEQ ID NOS 201 and 203, respectively, in order of appearance.

FIG. 6 shows exemplary plasmids encoding the APP695 isoform that each independently comprise a potential mutation, listed in TABLE 1, for use in expressing a mutant APPs in cells. The bi-directional CMV promoter can drive expression of mCherry and APP 695 from both sides of the promoter

FIG. 7 shows a cartoon denoting the target nucleotide mutations in APP used to generate modified APP proteins. The BACE and ADAM10 cleavage sites are indicated. The boxed nucleotides indicate target mutations. Figure discloses SEQ ID NOS 207-208, 207, 207, and 209-218, respectively, in order of appearance.

FIG. 8 shows a cartoon denoting the target nucleotide mutations in APP used to generate modified APP proteins. The BACE and ADAM10 cleavage sites are indicated. The boxed nucleotides indicate target mutations. Figure discloses SEQ ID NOS 219-220, 219, 219, and 221-225, respectively, in order of appearance.

FIG. 9 shows western blots for isolated cell lines with successful APP knock out. On the left side of the image, treatment with antibodies for expressed APP (Biolegend clone LN27, 1:1000 dilution, expected size 110 kDa) is shown. The right side of the image shows treatment with antibodies for GAPDH for loading control (Biolegend clone FF26A/F9, 1:1000 dilution, expected size 36 kDa). Successful targeting utilizing a CRISPR/Cas9 ribonucleoprotein (RNP) reduces or eliminates the expression of APP, as shown in cell lines 22-26.

FIG. 10 shows mRNA expression levels of different APP constructs relative to those of HPRT in APP KO HEK293 cells (see FIG. 9) transfected with plasmids containing target APP variants.

FIG. 11A shows a bar graph of the Abeta 40 protein expression levels (detected by ELISA) normalized to the APP mRNA expression level in APP KO HEK293 cells (see FIG. 9) transfected with plasmids containing target APP variants. The K670G, K670R+M671V, K670E+M671V, K670G+M671V, and M671V APP proteins exhibit statistically significant decreased expression level of Abeta 40 protein, as compared to the control (KO+WT-APP). The A673V APP protein exhibits statistically significant increased expression level, as compared to the control. Data represents mean+/−SEM. One-way ANOVA with Dunnett's posthoc test. * p<0.05; ** p<0.005, *** P<0.001, **** P<0.0001.

FIG. 11B is a magnified version of FIG. 11A, focusing on the five constructs with decreased expression compared to the control. Data represents mean+/−SEM. One-way ANOVA with Dunnett's posthoc test. * p<0.05; ** p<0.005, *** P<0.001.

FIG. 12A shows a bar graph of the Abeta 42 protein expression levels (detected by ELISA) normalized to APP mRNA expression level in APP KO HEK293 cells (see FIG. 9) transfected with plasmids containing target APP variants. The K670E, K670R+M671V, K670E+M671V, K670G+M671V, and M671V APP proteins exhibit statistically significant decreased expression level, as compared to the control (KO+WT-APP). The A673V APP protein exhibits statistically significant increased expression level, as compared to the control. Data represents mean+/−SEM. One-way ANOVA with Dunnett's correction for multiple comparisons. * p<0.05; ** p<0.005, *** P<0.001, **** P<0.0001.

FIG. 12B is a magnified version of FIG. 12A, focusing on the five constructs with decreased expression compared to the control. Data represents mean+/−SEM. One-way ANOVA with Dunnett's correction for multiple comparisons. * p<0.05; ** p<0.005, *** P<0.001.

FIGS. 13A-13D show U7-driven expression of engineered guide RNAs with a 3′ SmOPT and U7 hairpin that enhance specific guide RNA editing at additional gene targets with minimal unintended exon skipping. FIG. 13A shows the exon structure of human RAB7A and SNCA. Exons are shown as gray segments; the coding region is denoted as a black line above. Locations of the guide RNA targeting sites are shown in purple; PCR primers are shown in green. FIG. 13B shows ADAR editing at each target site (measured by Sanger sequencing). FIG. 13C shows cDNA from edited transcripts were PCR amplified using the above primers and analyzed on an agarose gel. PCR amplicons showed the predicted size for correctly spliced exons. FIG. 13D shows Sanger sequencing chromatograms show specific editing at the target adenosine of the indicated transcripts. Figure discloses SEQ ID NOS 202, 202, 202, 202, 202, 202, 202, 204, 204, 204-205, 204, 204, and 204, respectively, in order of appearance.

FIGS. 14A-14C show editing of the 3′ UTR of SNCA. FIG. 14A shows an example Sanger sequencing chromatogram of the edited sites of the 3′ UTR, as well as, off-target editing that can occur. Figure discloses SEQ ID NO: 206. FIG. 14B shows the mouse or human U7 promoter with 3′ SmOPT U7 hairpin constructs of the human SNCA 3′UTR target site, with or without ADAR2 overexpression, in a different cell type (K562-VPR-SNCA) under different transfection conditions (nucleofection, Lonza). FIG. 14C shows the percentage of off target editing occurring at the 5′G in the 3′ UTR using the same constructs as FIG. 14B.

FIG. 15 shows a bar graph of the gRNA mediated SNCA protein expression reductions (detected by ELISA) normalized to the WT SNCA expression level in HEK293 cells with a mock gRNA. The cells were transfected with plasmids containing target engineered polynucleotides Guide A, Guide B, two shRNAs (shRNA1 and shRNA2) targeting the SNCA protein, and the mock gRNA. Guide A and Guide B target the start codon and 3′UTR of the SNCA mRNA, respectively. The two guides also contain different features. These features and the sequences of Guide A and Guide B are listed in TABLE 14. Guide A and Guide B decreased the abundance of the SNCA protein by about 65% and 40%, when compared to the wildtype SNCA protein, respectively. In comparison, two shRNAs (shRNA1 and shRNA2) targeting the SNCA protein knocked down its expression by about 90%-99%.

FIG. 16A-FIG. 16B show RNA editing of APP using engineered polynucleotides targeting different regions of the APP transcript. FIG. 16A shows a cartoon schematic of the targeting strategy of different engineered polynucleotide. 0.100.50 (Exon-Exon) (SEQ ID NO: 97) is specific to the APP mRNA because it targets the continuous sequence across the exon with the target adenosine and its preceding exon. 0.100.50 (Exon-Intron) (SEQ ID NO: 98) is specific to the APP pre-mRNA because it targets the continuous sequence between the exon with the target adenosine and its preceding intron. 0.90.45 (Exon only) (SEQ ID NO: 99) can target both APP pre-mRNA and mRNA because it only targets the sequence of the target adenosine. White rectangles denote exons of the APP transcripts. Diagonal stripped rectangle denotes intron of the APP transcript. The targeting sequence of the engineered polynucleotide is shown as a black line above the APP transcript. FIG. 16B shows a bar graph of the RNA editing efficiency using the engineered polynucleotides in FIG. 16A and their negative controls (GFP plasmid and no transfection). Wild type HEK293 cells were transfected with plasmids encoding different polynucleotides or GFP. The RNA editing of APP mRNA was analyzed 48 hours after transfection. About 15-20% editing of the target adenosine in APP was achieved using the engineered polynucleotides in FIG. 16A, as compared to less than 3% editing in the negative controls.

FIG. 17A-FIG. 17C show an exemplary assay to screen for any of the engineered polynucleotides provided herein, including but not limited to SNCA, APP, and Tau. FIG. 17A shows a cartoon schematic of a luciferase reporter as the unedited target (ATG) and edited control (GTG). The reporter contains two open reading frames, each with one kozak and ATG start codon. The first open reading frame contains a target RNA sequence, while the second one contains a secreted luciferase. Deamination of the adenosine within the target start codon promotes secondary translation of luciferase proportional to the rate of editing. By incorporating the target RNA sequence surrounding the first ATG, the RNA editing efficiencies of the guides with different features—such as the length of the guide, the location of the bulge in the guide, or the location of the GLUR2-recruiting domains in the guide can be tested. FIG. 17B-FIG. 17C describes the results of one such experiment; FIG. 17B shows a heatmap of the luciferase expression of a fPMP22 reporter in response to a multitude of varied ADAR guides. The fPMP22 reporter was generated by inserting the target transcript sequence in Charcot-Marie-Tooth Syndrome 1A into the first open reading frame. The reporter was transposed into HEK293 cells for stable expression. Guides of different lengths (20, 30, 40, 50, 75, 100, 150, and 200 nucleotides), mismatch placement (10th percentile (5′end), 90^{th} percentile (3′ end), or 50^{th} percentile (middle)) of the guide as it's transcribed from 5′ to 3′ from the plasmid are listed in TABLE 16. The fold-change of the luciferase expression normalized to that of cells transfected with plasmids with non-specific guides and ADAR2 (ST0145). FIG. 17C shows a line graph of the relationship of the guide length (x-axis) and the fold-change of the reporter expression (y-axis) in two biological replicates for each guide. The result of 3 sets of experiments, in which the guide contained a GLUR2-recruiting domain in the 3′ end of the guide and the location of the bulge was varied, was shown.

FIG. 18A and FIG. 18B show the generation of ADAR1 knockout cell lines. FIG. 18A shows a carton schematic of the gene structure of ADAR1 and knockout strategy. Two gRNAs US gRNA and DS gRNA were designed to cover a 6 kb region of the ADAR1 locus, encompassing the deaminase domain (789^{th} to 1221^{st} amino acid). The nicking of the DNA strands directed by the gRNAs, combining with a homology directed repair (HDR) oligo with 80 bp homology arms outside the 6 kb region, creates the 6 kb deletion in the ADAR1 locus, removing the deaminase domain. FIG. 18B shows a western blot of ADAR1 in different clones transfected with US and DS gRNA. GAPDH was used as a control. Clones #9 and #11 showed no detectable ADAR1 protein expression by the western blot.

FIG. 19A and FIG. 19B show the generation of ADAR2 knockout cell lines. FIG. 19A shows a carton schematic of the gene structure of ADAR2 and knockout strategy. Two gRNAs US gRNA and DS gRNA were designed to cover a 9.5 kb region of the ADAR1 locus, encompassing the deaminase domain (70^{th} to 522^{nd} amino acid). The nicking of the DNA strands directed by the gRNAs, combining with a homology directed repair (HDR) oligo with 80 bp homology arms outside the 9.5 kb region, creates the 9.5 kb deletion in the ADAR2 locus, removing the deaminase domain. FIG. 19B shows a bar-graph of Q-PCR of ADARs in different clones transfected with US and DS gRNA.

FIG. 20A and FIG. 20B show the generation of ADAR1 knockout cell lines that overexpress (OE) ADAR2. FIG. 20A shows a carton schematic of the strategy to generate the ADAR1 knockout (KO) cell line that overexpresses ADAR2. An ADAR2 overexpression construct, maintained as a PiggyBac transposon with a puromycin-resistant marker, was transfected and integrated into an ADAR1 KO K562 cell line. The successfully integrated cell was selected by puromycin resistance.

FIG. 20B shows a western blot of ADAR1 and ADAR2 protein expression in wildtype, ADAR1 KO, and ADAR1 KO+ADAR2 cell. GAPDH was used as a control. The wildtype or ADAR1 KO cell did not express ADAR2. Only the ADAR1 KO cell successfully integrated with ADAR2 OE construct expressed ADAR2.

FIG. 21 shows a carton schematic of using a Bio-Rad drop-off digital droplet PCR (ddPCR) assay to measure RNA editing efficiency. PCR samples are prepared and then processed on fluidic chips to generate droplets of PCR reactions in oil suspension. The target and background reference sequences are detected: 1, by PCR amplification with intercalating fluorescent dyes; or 2, by fluorescent TaqMan style probes on the PCR amplified products. The resultant signal is analyzed by a droplet reader. Data is then presented in a two-dimensional dot plot, showing high and low populations of droplets for each fluorescent channel.

FIG. 22A and FIG. 22B show a carton schematic of using the drop-off ddPCR assay in FIG. 22 to measure RNA editing efficiency in human cells. FIG. 22A shows a carton schematic of using different DNA probe sets in the drop-off ddPCR assay to measure RNA editing efficiency. A forward and reverse primer are designed to flank the genomic locus or mRNA target of interest. A drop-off probe and reference TaqMan probe is designed to bind a target site and the region adjacent to the target site, respectively. Both probes can bind the wildtype sequence of the target site and the adjacent site to release signals; the drop-off probe cannot bind an edited or mutated sequence on the target site to release the signal. One of each target molecules, drop-off probes, and reference probes are allocated into one droplet. The percentage of the populations of the edited and wildtype target genomic loci or mRNAs, measured by the drop-off ddPCR, can be used to determine the frequency of editing. FIG. 22B depicts two dot-plots showing Rab7A RNA editing in human cells. Each Rab7A mRNA molecule was converted to a cDNA molecule by reverse transcription and PCR amplification, and allocated into individual droplet. Two TaqMan probes were designed for the target site and the reference site, bind the wildtype but not the edited sequences to provide signal. One of each target molecules, Drop-off probes, reference probes are allocated into one droplet. The signal intensity for each probe was measured for each droplet. In the wildtype control (WT) sample, most droplets showed high fluorescent intensity for both probes. In the edited sample, about 85% of the droplets showed decreased fluorescent intensity in the Drop-off probe, suggesting that an equivalent percentage of sequences were edited.

FIG. 23 shows a carton schematic of the design of universal gRNA quantification (gRNA^{Q}) tags that are added to the 5′ and 3′ ends of a guide RNA. These universal sequences allow for detection of any guide RNA inserted between the tags with addition of a guide specific TaqMan probe. In qPCR or ddPCR the primers will bind the gRNA^{Q} tags for amplification. The guide specific TaqMan probe will the produce a fluorescent signal that can be quantified using a standard curve with qPCR or ddPCR.

FIG. 24A and FIG. 24B show using the gRNA^{Q} assay in FIG. 23 to measure gRNA abundance. FIG. 24A shows the result of the quantification of Rab7A gRNA with gRNA^{Q} tags and GRAPH mRNA in a ddPCR reaction. The total number of positive droplets for Rab7 gRNA and GAPGH were counted and then analyzed by Poisson distribution to determine the frequency of all events. FIG. 24B shows the dot plots demonstrating that multiple serial dilutions of the sample to obtain amplification of GAPDH mRNA and gRNA in the sample. Since a dominant target can use up all the amplification reagents, diluting the sample can ensure that every target, even one with minute amount, is adequately amplified.

FIG. 25A and FIG. 25B show using different RNA editing using gRNAs with different structures. FIG. 25A shows a carton schematic of the RNA structures of different gRNAs against Rab7a. gRNA A 0.100.50 does not comprise any GluR2 loop to recruit ADAR. gRNA B 2.100.50 comprise two GluR2 loop on each end. gRNA C comprises two gRNA quantification tag (gRNAQ tag) of FIGS. 23 and 24. gRNA D comprises one 0.100.50 (gRNA A), and a gRNA^{Q} tag and a U6.27 protective loop on each end. FIG. 25B shows the RNA editing efficiencies of using the gRNAs of FIG. 25A. Co-expressing ADAR2 with gRNA B and D could increase the Rab7a RNA editing efficiency.

FIG. 26 shows a bar graph of the optimization of mRNA editing with the expression of gRNAs using different enhancer elements. The guide RNA, listed in SEQ ID NO: 124, was designed to target the Rab7a mRNA. It was driven by a hU6 promoter. Two configurations of the CMV enhancer were oriented against the hU6 promoter in several of the constructs and listed in TABLE 17. The constructs were designed to co-express ADAR2 or GFP as well as two GluRD domains on the 5′ and 3′ ends of the guide targeting Rab7a. ˜20,000 HEK293 cells were transfected with 1 μg of plasmid expressing SEQ ID NO: 124. Total RNA was collected 48 hours post transfection. Three biological replicates were tested.

FIG. 27 shows the three-dimensional structure of ADAR2 double-stranded RNA-binding domain (dsRBD) binding to the GluR2 hairpin binding of the gRNA/target RNA complex. The editing adenosine site is shown.

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. Unless defined otherwise, all technical and scientific terms used herein have the same meaning as is commonly understood by one of skill in the art.

The term “a” and “an” refers to one or to more than one (i.e., to at least one) of the grammatical object of the article. By way of example, “an element” means one element or more than one element.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “partially”, “at least partially”, or as used herein can refer to a value approaching 100% of a given value. In some cases, the term can refer to an amount that can be at least about 60%, 65%, 70%, 75%, 80%, 85%, 90%, 91%, 92%, 93%, 94%, 95%, 96%, 97%, 98%, 99%, 99.9%, or 99.99% of a total amount. In some cases, the term can refer to an amount that can be about 100% of a total amount.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “subject,” “host,” “individual,” and “patient” are as used interchangeably herein to refer to animals, typically mammalian animals. Any suitable mammal can be treated by a method, cell or composition described herein. A mammal can be administered a vector, an engineered guide RNA, a precursor guide RNA, a nucleic acid, or a pharmaceutical composition, as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a disease such as a neurodegenerative disease. In some embodiments, a subject has or can be suspected of having a cancer or neoplastic disorder. In other embodiments, a subject has or can be suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

The term “subject,” “host,” “individual,” and “patient” are as used interchangeably herein to refer to animals, typically mammalian animals. Any suitable mammal can be administered a composition as described herein (such as an engineered guide RNA) or treated by a method as described herein. A subject can be a vertebrate or an invertebrate. A subject can be a laboratory animal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

The term “protein”, “peptide”, and “polypeptide” are used interchangeably and in their broadest sense to refer to a compound of two or more subunit amino acids, amino acid analogs or peptidomimetics. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The terms “polynucleotide” and “oligonucleotide” are used interchangeably and refer to a polymeric form of nucleotides of any length, either deoxyribonucleotides or ribonucleotides or analogs thereof. Polynucleotides can have any three dimensional structure and may perform any function, known or unknown. The following are non-limiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA). A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction. Unless otherwise specified or required, any embodiment of this disclosure that is a polynucleotide encompasses both the double stranded form and each of two complementary single stranded forms known or predicted to make up the double stranded form.

Polynucleotides useful in the methods of the disclosure can comprise natural nucleic acid sequences and variants thereof, artificial nucleic acid sequences, or a combination of such sequences. In some embodiments, polynucleotides of the disclosure refer to a DNA sequence. In some embodiments, the DNA sequence is interchangeable with a similar RNA sequence. In some embodiments, polynucleotides of the disclosure refer to an RNA sequence. In some embodiments, the RNA sequence is interchangeable with a similar DNA sequence. In some embodiments, Us and Ts of a polynucleotide may be interchanged in a sequence provided herein.

A polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9 glycosidic bond, resulting in the chemical structure:

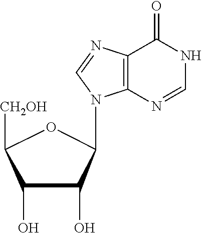

Inosine is read by the translation machinery as guanine (G).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The terms “equivalent” or “biological equivalent” are used interchangeably when referring to a particular molecule, biological, or cellular material and intend those having minimal homology while still maintaining desired structure or functionality.

The term “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (i.e., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation may indicate that a tissue or sample is benign. As an alternative, absence of a mutation may not indicate that a tissue or sample is benign. Methods as described herein can comprise identifying a presence of a mutation in a sample.

“Canonical amino acids” refer to those 20 amino acids found naturally in the human body shown in the table below with each of their three letter abbreviations, one letter abbreviations, structures, and corresponding codons:

non-polar, aliphatic residues

| | | | | |
|---|---|---|---|---|
| Glycine | Gly | G | 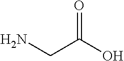 | GGU GGC GGA GGG |
| Alanine | Ala | A | 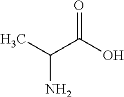 | GCU GCC GCA GCG |
| Valine | Val | V | 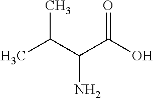 | GUU GUC GUA GUG |
| Leucine | Leu | L | 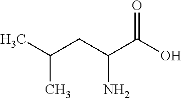 | UUA UUG CUU CUC CUA CUG |
| Isoleucine | Ile | I | 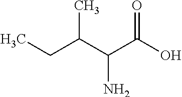 | AUU AUC AUA |
| Proline | Pro | P | 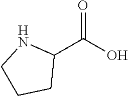 | CCU CCC CCA CCG |

aromatic residues

| | | | | |
|---|---|---|---|---|
| Phenylalanine | Phe | F | 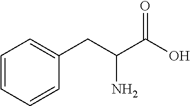 [SMILES: NC(Cc1ccccc1)C(=O)O] | UUU UUC |
| Tyrosine | Tyr | Y | 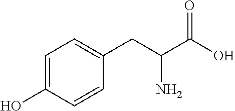 [SMILES: NC(Cc1ccc(O)cc1)C(=O)O] | UAU UAC |
| Tryptophan | Trp | W | 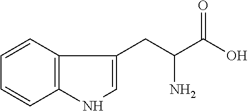 [SMILES: NC(Cc1c[nH]c2ccccc12)C(=O)O] | UGG |

polar, non-charged residues

| | | | | |
|---|---|---|---|---|
| Serine | Ser | S | 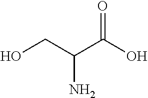 | UCU UCC UCA UCG AGU AGC |
| Threonine | Thr | T | 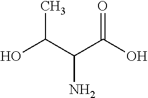 | ACU ACC ACA ACG |
| Cysteine | Cys | C | 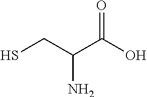 | UGU UGC |
| Methionine | Met | M | 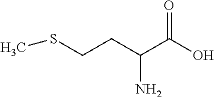 | AUG |
| Asparagine | Asn | N | 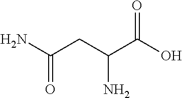 | AAU AAC |
| Glutamine | Gin | Q | 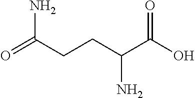 | CAA CAG |

positively charged residues

| | | | | |
|---|---|---|---|---|
| Lysine | Lys | K | 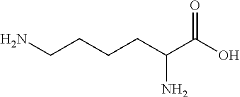 | AAA AAG |
| Arginine | Arg | R | 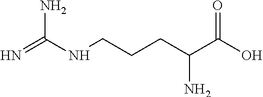 [SMILES: N=C(N)NCCCC(N)C(=O)O] | CGU CGC CGA CGG AGA AGG |
| Histidine | His | H | 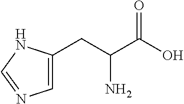 [SMILES: NC(Cc1cnc[nH]1)C(=O)O] | CAU CAC |

negatively charged residues

| | | | | |
|---|---|---|---|---|
| Aspartate | Asp | D | 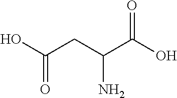 | GAU GAC |
| Glutamate | Glu | E | 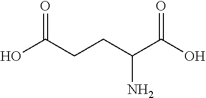 | GAA GAG |

The term “non-canonical amino acids” refers to those synthetic or otherwise modified amino acids that fall outside this group, typically generated by chemical synthesis or modification of canonical amino acids (e.g. amino acid analogs). The present disclosure employs proteinogenic non-canonical amino acids in some of the methods and vectors disclosed herein. A non-limiting exemplary non-canonical amino acid is pyrrolysine (Pyl or O), the chemical structure of which is provided below:

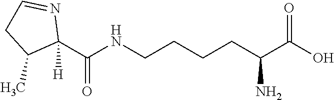 [SMILES: C[C@@H]1CC=N[C@H]1C(=O)NCCCC[C@H](N)C(=O)O]

Inosine (I) is another exemplary non-canonical amino acid, which is commonly found in tRNA and is essential for proper translation according to “wobble base pairing.” The structure of inosine is provided above.

The term “complementary” or “complementarity” refers to the ability of a nucleic acid to form hydrogen bond(s) with another nucleic acid sequence by either traditional Watson-Crick or other non-traditional types. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary”, “partially complementary”, “at least partially complementary”, or as used herein refers to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or refers to two nucleic acids that hybridize under stringent conditions (e.g., stringent hybridization conditions). Nucleic acids can include nonspecific sequences. As used herein, the term “nonspecific sequence” or “not specific” refers to a nucleic acid sequence that contains a series of residues that can be not designed to be complementary to or can be only partially complementary to any other nucleic acid sequence.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement. The section headings used herein are for organizational purposes and are not to be construed as limiting the subject matter described.

### Overview

RNA editing has emerged as an attractive alternative to DNA editing. Unlike DNA editing, RNA editing can be less likely to cause a potentially dangerous immune reaction such as those reported utilizing CRISPR-based approaches. Indeed, unlike the DNA-editing enzyme Cas9, which comes from bacteria, RNA editing entities and biologically active fragments thereof such as Adenosine Deaminase Acting on RNA (ADAR) are human proteins that do not trigger the adaptive immune system. Additionally, RNA editing may be a safer approach to gene therapies because editing RNA does not contain a risk for permanent genomic changes as seen with DNA editing. Also, while off-site RNA editing may occur, the off-site edited mRNA is diluted out and/or degraded, unlike with off-site DNA editing that is permanent, e.g., the transient nature of mRNA compared to the permeance of DNA, off-site editing is likely far less consequential in the context of RNA vs DNA.

Provided herein are compositions of engineered polynucleotides (also referred to as “engineered guide RNAs (gRNAs)” or “guide RNAs”) and methods for use in targeting an RNA, particularly for the prevention, amelioration, and/or treatment of disease. Although many diseases can be targeted utilizing the compositions and methods provided herein, those associated with mutations in Amyloid Precursor Protein (APP) are preferentially targeted. APP mutations are associated with diseases arising in the central nervous system (CNS). In an aspect, the compositions and methods of the disclosure provide suitable means for which to treat CNS disease with improved targeting and reduced immunogenicity as compared to available technologies utilizing DNA editing.

In some cases, amyloid precursor protein (APP) can be cut by beta secretase (BACE) or gamma secretase, and the fragment resulting from such cuts can be Amyloid beta (referred to as “Aβ” or “Abeta”) peptides of 36-43 amino acids. Certain Abeta peptide metabolites of this cleavage can be crucially involved in Alzheimer's disease pathology and progression.

Compositions described herein can edit the cleavage site in APP, so that beta/gamma secretases exhibit reduced cleavage of APP or can no longer cut APP and, therefore, reduced levels of Abeta 40/42 or no Abetas can be produced.

Also disclosed herein are compositions of engineered polynucleotides and methods of use thereof for targeting RNA to knockdown proteins implicated in a neurodegenerative disease. For example, engineered polynucleotides disclosed herein may target Tau (e.g., a microtubule-associated protein Tau (MAPT) encoded from a MAPT gene) or α-synuclein (SNCA) for knockdown. Compositions disclosed herein also include combinations of more than one engineered guide RNA, for example, engineered guides targeting a cleavage site in APP for RNA editing and engineered guides targeting Tau or SNCA for knockdown. These disclosed compositions can have synergistic effects to prevent and/or cure a neurodegenerative disease. The compositions and methods disclosed herein can yield results in editing and/or knockdown of targets without any of the resulting issues seen in small molecule or antibody therapy. Compositions can partially knockdown APP (instead of target cleavage site editing). Editing at the target cleavage site in APP and partial knockdown can be deployed singly or in combination.

### Targeting of Ribonucleic Acid

Targeting an RNA can be a process by which RNA can be enzymatically modified post synthesis on specific nucleosides or bases.

Targeting of RNA can be a way to modulate expression of a polypeptide. For example, through modulation of polypeptide-encoding double stranded RNA (dsRNA) substrates that enter the RNA interference (RNAi) pathway. This modulation may then act at the chromatin level to modulate expression of the polypeptide.

Targeting of RNA can also be a way to regulate gene translation. RNA editing can be a mechanism in which to regulate transcript recoding by regulating the triplet codon to introduce silent mutations and/or non-synonymous mutations.

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code could in theory be reassigned through RNA editing. Due to the degeneracy of the genetic code, RNA editing can cause both silent and non-synonymous amino acid substitutions.

Targeting an RNA can chemically transform a base of a nucleotide in a targeted RNA. In some cases, targeting an RNA can affect splicing. Adenosines targeted for editing may be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine (G), and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In some embodiments, a engineered polynucleotide and a target RNA molecule can have at least one mismatch. In some embodiments, a engineered polynucleotide and a target RNA molecule can have one mismatch. In some embodiments, a engineered polynucleotide and a target RNA molecule can have an A/C, A/G, U/C, U/G, C/A, C/U, G/A, G/U mismatch, or any combination thereof. In some embodiments, a engineered polynucleotide and a target RNA molecule can have a A/C mismatch. In some embodiment, an A in an A/C, A/G, C/A, or G/A can be modified by the RNA editing entity. In other cases, a C in an A/C, C/A, C/U. or U/C can be modified by the RNA editing entity. In other cases, a U in a U/C, C/U, G/U. or U/G can be modified by the RNA editing entity. In other cases, a G in a U/G, G/U, G/A. or A/G can be modified by the RNA editing entity. In some embodiments, an A in an A/C mismatch can be modified by the RNA editing entity. Such modifications can comprise any modification, for example chemical modifications induced by any RNA editing entity described herein and thereof. In an embodiment, a modification reverts a mismatch in a target RNA to a residue present in an otherwise comparable WT RNA.

A chemical transformation of a targeted RNA can result in an increased level of a protein or fragment thereof after translation of the targeted RNA with the chemical transformation, relative to an otherwise comparable RNA lacking the chemical transformation. In some embodiments, a chemical transformation of a targeted RNA can result in a decreased level of a protein or fragment thereof after translation of the targeted RNA with the chemical transformation, relative to an otherwise comparable RNA lacking the chemical transformation. In some embodiments, a chemical transformation can convert a sense codon into a stop codon. In some cases, a chemical transformation can convert a stop codon into a sense codon. In some embodiments, a chemical transformation can convert a first sense codon into a second sense codon. In some instances, a chemical transformation can convert a first stop codon into a second stop codon. In some embodiments, a chemical transformation can alter the localization, folding, stability, or synthesis of a protein or fragment thereof after translation of a targeted RNA with the chemical transformation, relative to an otherwise comparable RNA lacking the chemical transformation. In some cases, a chemical transformation can alter the localization, folding, stability, or synthesis of a targeted RNA with the chemical transformation, relative to an otherwise comparable target RNA lacking the chemical transformation. In some embodiments, a target RNA can comprise a coding or a non-coding RNA.

Targeting of RNA can comprise any one of an insertion, deletion, or substitution of a base. Examples of RNA targeting include pseudouridylation (the isomerization of uridine residues) and deamination (removal of an amine group from cytidine to give rise to uridine, or C-to-U editing).

### RNA Editing Entities and Biologically Active Fragments Thereof

Provided herein are compositions that comprise an RNA editing entity or a biologically active fragment thereof and methods of using the same. In an aspect, an RNA editing entity can comprise an adenosine Deaminase Acting on RNA (ADAR), Adenosine Deaminase Acting on tRNA (ADAT), and biologically active fragments thereof of either of these. ADARs and ADATs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR and ADAT enzymes share a common domain architecture comprising a variable number of amino-terminal dsRNA binding domains (dsRBDs) and a single carboxy-terminal catalytic deaminase domain. Human ADARs and ADATs possess two or three dsRBDs. Evidence suggests that ADARs and ADATs can form homodimer as well as heterodimer with other ADARs or and ADATs when bound to double-stranded RNA, however it is currently inconclusive if dimerization is required for editing to occur.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. ADAT catalyzes the deamination on tRNAs. ADAT is also named tadA in E. coli. Three human ADAT genes have been identified (ADATs 1-3).

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code could be reassigned through RNA editing. Due to the degeneracy of the genetic code, RNA editing can cause both silent and non-synonymous amino acid substitutions.

In some cases, targeting an RNA can affect splicing. Adenosines targeted for editing may be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine, and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In an aspect, an RNA editing entity comprises an ADAR. In some embodiments, an ADAR can comprise any one of: ADAR1, ADAR1p110, ADAR1p150, ADAR2, ADAR3, APOBEC protein, or any combination thereof. In some embodiments, the ADAR RNA editing entity is ADAR1. Additionally, or alternatively, the ADAR RNA editing entity is ADAR2. Additionally, or alternatively, the ADAR RNA editing entity is ADAR3. In an aspect, an RNA editing entity can be a non-ADAR In some cases, an RNA editing entity can comprise at least about 80% sequence homology to APOBEC1, APOBEC2, ADAR1, ADAR1p110, ADAR1p150, ADAR2, ADAR3, or any combination thereof. Alternate editing entities are also contemplated, such as those from a clustered regularly interspaced short palindromic repeats (CRISPR) system.

In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA. In some instances, an RNA editing entity can comprise a recombinant enzyme. In some cases, an RNA editing entity can comprise a fusion polypeptide.

In an aspect, an RNA editing entity can be recruiting by a subject engineered polynucleotide. In some embodiments, an engineered polynucleotide can recruit an RNA editing entity that, when associated with the engineered polynucleotide and the target RNA or not associated with the target RNA, facilitates: an editing of a base of a nucleotide of a polynucleotide of the region of the target RNA, a modulation of the expression of a polypeptide encoded by a subject target RNA, such as APP, SNCA, Tau; or a combination thereof. An engineered polynucleotide can contain an RNA editing entity recruiting domain to be capable of recruiting an RNA editing entity.

### Engineered Polynucleotides

Provided herein are polynucleotides and compositions that comprise the same. In an aspect, a polynucleotide can be an engineered polynucleotide. In an embodiment, an engineered polynucleotide can be an engineered polynucleotide. In some embodiments, an engineered polynucleotide of the disclosure may be utilized for RNA editing, for example to prevent or treat a disease or condition. In some cases, an engineered polynucleotide can be used in association with a subject RNA editing entity to edit a target RNA or modulate expression of a polypeptide encoded by the target RNA. In an embodiment, compositions disclosed herein can include engineered polynucleotides capable of facilitating editing by subject RNA editing entities such as ADAR or ADAT polypeptides or biologically active fragments thereof.

Engineered polynucleotides can be engineered in any way suitable for RNA targeting. In an aspect, an engineered polynucleotide generally comprises at least a targeting sequence that allows it to hybridize to a region of a target RNA. In some cases, a targeting sequence may also be referred to as a targeting domain or a targeting region.

In an aspect, a targeting sequence of an engineered polynucleotide allows the engineered polynucleotide to target an RNA sequence through base paring, such as Watson Crick base pairing. In an embodiment, the targeting sequence can be located at either the N-terminus or C-terminus of the engineered polynucleotide. In some cases, the targeting sequence is located at both termini. The targeting sequence can be of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In an embodiment, an engineered polynucleotide comprises a targeting sequence that is about 75-100, 80-110, 90-120, or 95-115 nucleotides in length. In an embodiment, an engineered polynucleotide comprises a targeting sequence that is about 100 nucleotides in length.

In some cases, a subject targeting sequence comprises at least partial sequence complementarity to a region of a target RNA that at least partially encodes a subject polypeptide for example APP, SNCA, or Tau. In some cases, a targeting sequence comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence may have a single base mismatch. In other cases, the targeting sequence of a subject engineered polynucleotide comprises at least about 1, 2, 3, 4, 5, 6, 7, 8, 9, 10, 11, 12, 13, 14, 15, 16, 20, 30, 40 or up to about 50 base mismatches. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype RNA of a subject target RNA.

In an aspect, a subject engineered polynucleotide comprises an RNA editing entity recruiting domain. An RNA editing entity can be recruited by an RNA editing entity recruiting domain on an engineered polynucleotide. In some cases, a subject engineered polynucleotide is configured to facilitate editing of a base of a nucleotide of a polynucleotide of a region of a subject target RNA, modulation expression of a polypeptide encoded by the subject target RNA, or both. In some cases, an engineered polynucleotide can be configured to facilitate an editing of a base of a nucleotide or polynucleotide of a region of an RNA by a subject RNA editing entity. In order to facilitate editing, an engineered polynucleotide of the disclosure may recruit an RNA editing entity. In certain embodiments, an engineered polynucleotide lacks an RNA editing entity recruiting domain. Either way, a subject engineered polynucleotide can be capable of binding an RNA editing entity, or be bound by it, and facilitate editing of a subject target RNA.

Various RNA editing entity recruiting domains can be utilized. In an embodiment, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In certain embodiments, more than one recruiting domain can be included in an engineered polynucleotide of the disclosure. In cases where a recruiting sequence is present, the recruiting sequence can be utilized to position the RNA editing entity to effectively react with a subject target RNA after the targeting sequence, for example an antisense sequence, hybridizes to a target RNA. In some cases, a recruiting sequence can allow for transient binding of the RNA editing entity to the engineered polynucleotide. In other cases, the recruiting sequence allows for permanent binding of the RNA editing entity to the polynucleotide. A recruiting sequence can be of any length. In some cases, a recruiting sequence is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting sequence is about 45 nucleotides in length. In some cases, at least a portion of a recruiting sequence comprises at least 1 to about 75 nucleotides. In some cases, at least a portion of a recruiting sequence comprises about 45 nucleotides to about 60 nucleotides.

In an embodiment, an RNA editing entity recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In an embodiment, a recruiting domain comprises a GluR2 sequence, or a sequence having at least about 80%, 85%, 90%, 95%, 98%, 99%, or 100% identity to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some embodiments, a recruiting domain can comprise at least about 90%, 95%, 96%, 97%, 98%, or 99% sequence homology to SEQ ID NO: 1.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an MS2-bacteriophage-coat-protein-recruiting domain. In another embodiment, a recruiting domain can be from an Alu domain. In some cases, a recruiting domain can comprise at least about: 80%, 85%, 90%, or 95% sequence homology to at least about: 15, 20, 25, 30, or 35 nucleotides of an APOBEC, MS2-bacteriophage-coat-protein-recruiting domain, or Alu domain. Any number of recruiting sequences may be found in a polynucleotide of the present disclosure. In some cases, at least about 1, 2, 3, 4, 5, 6, 7, 8, 9, or up to about 10 recruiting sequences are included in a polynucleotide. Recruiting sequences may be located at any position of subject polynucleotides. In some cases, a recruiting sequence is on an N-terminus, middle, or C-terminus of a polynucleotide. A recruiting sequence can be upstream or downstream of a targeting sequence. In some cases, a recruiting sequence flanks a targeting sequence of a subject polynucleotide. A recruiting sequence can comprise all ribonucleotides or deoxyribonucleotides, although a recruiting sequence comprising both ribo- and deoxyribonucleotides is not excluded.

In cases where a recruiting sequence can be absent, an engineered polynucleotide can be still capable of associating with a subject RNA editing entity (e.g., ADAR) to facilitate editing of a target RNA and/or modulate expression of a polypeptide encoded by a subject target RNA. This can be achieved through structural features. Structural features can comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate, for example hybridized polynucleotide strands, can be formed upon hybridization of an engineered polynucleotide of the present disclosure to a target RNA. Described herein can be a feature, which corresponds to one of several structural features that can be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a recruiting hairpin or a hairpin comprising a non-targeting domain). Engineered polynucleotides of the present disclosure can have from 1 to 50 features. Engineered polynucleotides of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

As disclosed herein, a structured motif comprises two or more features in a dsRNA substrate.

A double stranded RNA (dsRNA) substrate can be formed upon hybridization of an engineered polynucleotide of the present disclosure to a target RNA. As disclosed herein, a mismatch refers to a nucleotide in a polynucleotide that can be unpaired to an opposing nucleotide in a target RNA within the dsRNA. A mismatch can comprise any two nucleotides that do not base pair, can be not complementary, or both. In some embodiments, a mismatch can be an A/C mismatch. An A/C mismatch can comprise a C in an engineered polynucleotide of the present disclosure opposite an A in a target RNA. An A/C mismatch can comprise a A in an engineered polynucleotide of the present disclosure opposite an C in a target RNA. In an embodiment, a G/G mismatch can comprise a G in an engineered polynucleotide of the present disclosure opposite a G in a target RNA. In some embodiments, a mismatch positioned 5′ of the edit site can facilitate base-flipping of the target A to be edited. A mismatch can also help confer sequence specificity. In an embodiment, a mismatch comprises a G/G mismatch. In an embodiment, a mismatch comprises an A/C mismatch, wherein the A can be in the target RNA and the C can be in the targeting sequence of the engineered polynucleotide. In another embodiment, the A in the A/C mismatch can be the base of the nucleotide in the target RNA edited by a subject RNA editing entity.

In an aspect, a structural feature can form in an engineered polynucleotide independently. In other cases, a structural feature can form when an engineered polynucleotide binds to a target RNA. A structural feature can also form when an engineered polynucleotide associates with other molecules such as a peptide, a nucleotide, or a small molecule. In certain embodiments, a structural feature of an engineered polynucleotide can be formed independent of a target RNA, and its structure can change as a result of the engineered polypeptide hybridization with a target RNA region. In certain embodiments, a structural feature can be present when an engineered polynucleotide can be in association with a target RNA.

In some cases, a structural feature can be a hairpin. In some cases, an engineered polynucleotide can lack a hairpin domain. In other cases, an engineered polynucleotide can contain a hairpin domain or more than one hairpin domain. A hairpin can be located anywhere in a polynucleotide. As disclosed herein, a hairpin can be an RNA duplex wherein a single RNA strand has folded in upon itself to form the RNA duplex. The single RNA strand folds upon itself due to having nucleotide sequences upstream and downstream of the folding region base pairs to each other. A hairpin can have from 10 to 500 nucleotides in length of the entire duplex structure. The stem-loop structure of a hairpin can be from 3 to 15 nucleotides long. A hairpin can be present in any of the engineered polynucleotides disclosed herein. The engineered polynucleotides disclosed herein can have from 1 to 10 hairpins. In some embodiments, the engineered polynucleotides disclosed herein have 1 hairpin. In some embodiments, the engineered polynucleotides disclosed herein have 2 hairpins. As disclosed herein, a hairpin can refer to a recruitment hairpin or a hairpin or a non-recruitment hairpin. A hairpin can be located anywhere within the engineered polynucleotides of the present disclosure. In some embodiments, one or more hairpins can be present at the 3′ end of an engineered polynucleotide of the present disclosure, at the 5′ end of an engineered polynucleotide of the present disclosure or within the targeting sequence of an engineered polynucleotide of the present disclosure, or any combination thereof.

In aspect, a structural feature comprises a recruitment hairpin, as disclosed herein. A recruitment hairpin can recruit an RNA editing entity, such as ADAR. In some embodiments, a recruitment hairpin comprises a GluR2 domain. In some embodiments, a recruitment hairpin comprises an Alu domain.

In yet another aspect, a structural feature comprises a non-recruitment hairpin. A non-recruitment hairpin, as disclosed herein, can exhibit functionality that improves localization of the engineered polynucleotide to the target RNA. In some embodiments, the non-recruitment hairpin improves nuclear retention. In some embodiments, the non-recruitment hairpin comprises a hairpin from U7 snRNA.

In another aspect, a structural feature comprises a wobble base. A wobble base pair refers to two bases that weakly pair. For example, a wobble base pair of the present disclosure can refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides. A hairpin can be a structural feature formed from a single strand of RNA with sufficient complementarity to itself to hybridize into a double stranded RNA motif/structure consisting of double-stranded hybridized RNA separated by a nucleotide loop.

In some cases, a structural feature can be a bulge. A bulge can comprise a single (intentional) nucleic acid mismatch between the target strand and an engineered polynucleotide strand. In other cases, more than one consecutive mismatch between strands constitutes a bulge as long as the bulge region, mismatched stretch of bases, can be flanked on both sides with hybridized, complementary dsRNA regions. A bulge can be located at any location of a polynucleotide. In some cases, a bulge can be located from about 30 to about 70 nucleotides from a 5′ hydroxyl or the 3′ hydroxyl.

In an embodiment, a double stranded RNA (dsRNA) substrate can be formed upon hybridization of an engineered polynucleotide of the present disclosure to a target RNA. As disclosed herein, a bulge refers to the structure formed upon formation of the dsRNA substrate, where nucleotides in either the engineered polynucleotide or the target RNA can be not complementary to their positional counterparts on the opposite strand. A bulge can change the secondary or tertiary structure of the dsRNA substrate. A bulge can have from 1 to 4 nucleotides on the engineered polynucleotide side of the dsRNA substrate or the target RNA side of the dsRNA substrate. In some embodiments, the engineered polynucleotides of the present disclosure have 2 bulges. In some embodiments, the engineered polynucleotides of the present disclosure have 3 bulges. In some embodiments, the engineered polynucleotides of the present disclosure have 4 bulges. In some embodiments, the presence of a bulge in a dsRNA substrate can position ADAR to selectively edit the target A in the target RNA and reduce off-target editing of non-targets. In some embodiments, the presence of a bulge in a dsRNA substrate can recruit additional ADAR. Bulges in dsRNA substrates disclosed herein can recruit other proteins, such as other RNA editing entities. In some embodiments, a bulge positioned 5′ of the edit site can facilitate base-flipping of the target A to be edited. A bulge can also help confer sequence specificity. A bulge can help direct ADAR editing by constraining it in an orientation that yield selective editing of the target A.

In an aspect, a double stranded RNA (dsRNA) substrate can be formed upon hybridization of an engineered polynucleotide of the present disclosure to a target RNA. A bulge can be a symmetrical bulge or an asymmetrical bulge. A bulge can be formed by 1 to 4 participating nucleotides on either the polynucleotide side or the target RNA side of the dsRNA substrate. A symmetrical bulge can be formed when the same number of nucleotides can be present on each side of the bulge. A symmetrical bulge can have from 2 to 4 nucleotides on the engineered polynucleotide side of the dsRNA substrate or the target RNA side of the dsRNA substrate. For example, a symmetrical bulge in a dsRNA substrate of the present disclosure can have the same number of nucleotides on the engineered polynucleotide side and the target RNA side of the dsRNA substrate. A symmetrical bulge of the present disclosure can be formed by 2 nucleotides on the engineered polynucleotide side of the dsRNA target and 2 nucleotides on the target RNA side of the dsRNA substrate. A symmetrical bulge of the present disclosure can be formed by 3 nucleotides on the engineered polynucleotide side of the dsRNA target and 3 nucleotides on the target RNA side of the dsRNA substrate. A symmetrical bulge of the present disclosure can be formed by 4 nucleotides on the engineered polynucleotide side of the dsRNA target and 4 nucleotides on the target RNA side of the dsRNA substrate.

A double stranded RNA (dsRNA) substrate can be formed upon hybridization of an engineered polynucleotide of the present disclosure to a target RNA. A bulge can be a symmetrical bulge or an asymmetrical bulge. An asymmetrical bulge can be formed when a different number of nucleotides can be present on each side of the bulge. An asymmetrical bulge can have from 1 to 4 participating nucleotides on either the polynucleotide side or the target RNA side of the dsRNA substrate. For example, an asymmetrical bulge in a dsRNA substrate of the present disclosure can have different numbers of nucleotides on the engineered polynucleotide side and the target RNA side of the dsRNA substrate. An asymmetrical bulge of the present disclosure can be formed by 0 nucleotides on the engineered polynucleotide side of the dsRNA substrate and 1 nucleotide on the target RNA side of the dsRNA substrate. An asymmetrical bulge of the present disclosure can be formed by 0 nucleotides on the target RNA side of the dsRNA substrate and 1 nucleotide on the engineered polynucleotide side of the dsRNA substrate. An asymmetrical bulge of the present disclosure can be formed by 0 nucleotides on the engineered polynucleotide side of the dsRNA substrate and 2 nucleotides on the target RNA side of the dsRNA substrate. An asymmetrical bulge of the present disclosure can be formed by 0 nucleotides on the target RNA side of the dsRNA substrate and 2 nucleotides on the engineered polynucleotide side of the dsRNA substrate. An asymmetrical bulge of the present disclosure can be formed by 0 nucleotides on the engineered polynucleotide side of the dsRNA substrate and 3 nucleotides on the target RNA side of the dsRNA substrate. An asymmetrical bulge of the present disclosure can be formed by 0 nucleotides on the target RNA side of the dsRNA substrate and 3 nucleotides on the engineered polynucleotide side of the dsRNA substrate. An asymmetrical bulge of the present disclosure can be formed by 0 nucleotides on the engineered polynucleotide side of the dsRNA substrate and 4 nucleotides on the target RNA side of the dsRNA substrate. An asymmetrical bulge of the present disclosure can be formed by 0 nucleotides on the target RNA side of the dsRNA substrate and 4 nucleotides on the engineered polynucleotide side of the dsRNA substrate. An asymmetrical bulge of the present disclosure can be formed by 1 nucleotide on the engineered polynucleotide side of the dsRNA substrate and 2 nucleotides on the target RNA side of the dsRNA substrate. An asymmetrical bulge of the present disclosure can be formed by 1 nucleotide on the target RNA side of the dsRNA substrate and 2 nucleotides on the engineered polynucleotide side of the dsRNA substrate. An asymmetrical bulge of the present disclosure can be formed by 1 nucleotide on the engineered polynucleotide side of the dsRNA substrate and 3 nucleotides on the target RNA side of the dsRNA substrate. An asymmetrical bulge of the present disclosure can be formed by 1 nucleotide on the target RNA side of the dsRNA substrate and 3 nucleotides on the engineered polynucleotide side of the dsRNA substrate. An asymmetrical bulge of the present disclosure can be formed by 1 nucleotides on the engineered polynucleotide side of the dsRNA substrate and 4 nucleotides on the target RNA side of the dsRNA substrate. An asymmetrical bulge of the present disclosure can be formed by 1 nucleotide on the target RNA side of the dsRNA substrate and 4 nucleotides on the engineered polynucleotide side of the dsRNA substrate. An asymmetrical bulge of the present disclosure can be formed by 2 nucleotides on the engineered polynucleotide side of the dsRNA substrate and 3 nucleotides on the target RNA side of the dsRNA substrate. An asymmetrical bulge of the present disclosure can be formed by 2 nucleotides on the target RNA side of the dsRNA substrate and 3 nucleotides on the engineered polynucleotide side of the dsRNA substrate. An asymmetrical bulge of the present disclosure can be formed by 2 nucleotides on the engineered polynucleotide side of the dsRNA substrate and 4 nucleotides on the target RNA side of the dsRNA substrate. An asymmetrical bulge of the present disclosure can be formed by 2 nucleotides on the target RNA side of the dsRNA substrate and 4 nucleotides on the engineered polynucleotide side of the dsRNA substrate. An asymmetrical bulge of the present disclosure can be formed by 3 nucleotides on the engineered polynucleotide side of the dsRNA substrate and 4 nucleotides on the target RNA side of the dsRNA substrate. An asymmetrical bulge of the present disclosure can be formed by 3 nucleotides on the target RNA side of the dsRNA substrate and 4 nucleotides on the engineered polynucleotide side of the dsRNA substrate. In some cases, a structural feature can be a loop.

In an aspect, a double stranded RNA (dsRNA) substrate can be formed upon hybridization of an engineered polynucleotide of the present disclosure to a target RNA. As disclosed herein, an internal loop refers to the structure formed upon formation of the dsRNA substrate, where nucleotides in either the engineered polynucleotide or the target RNA can be not complementary to their positional counterparts on the opposite strand and where one side of the internal loop, either on the target RNA side or the engineered polynucleotide side of the dsRNA substrate, has greater than 5 nucleotides. An internal loop can be a symmetrical internal loop or an asymmetrical internal loop. Internal loops present in the vicinity of the edit site can help with base flipping of the target A in the target RNA to be edited. A double stranded RNA (dsRNA) substrate can be formed upon hybridization of an engineered polynucleotide of the present disclosure to a target RNA. An internal loop can be a symmetrical internal loop or an asymmetrical internal loop. A symmetrical internal loop can be formed when the same number of nucleotides can be present on each side of the internal loop. For example, a symmetrical internal loop in a dsRNA substrate of the present disclosure can have the same number of nucleotides on the engineered polynucleotide side and the target RNA side of the dsRNA substrate. A symmetrical internal loop of the present disclosure can be formed by 5 nucleotides on the engineered polynucleotide side of the dsRNA target and 5 nucleotides on the target RNA side of the dsRNA substrate. A symmetrical internal loop of the present disclosure can be formed by 6 nucleotides on the engineered polynucleotide side of the dsRNA target and 6 nucleotides on the target RNA side of the dsRNA substrate. A symmetrical internal loop of the present disclosure can be formed by 7 nucleotides on the engineered polynucleotide side of the dsRNA target and 7 nucleotides on the target RNA side of the dsRNA substrate. A symmetrical internal loop of the present disclosure can be formed by 8 nucleotides on the engineered polynucleotide side of the dsRNA target and 8 nucleotides on the target RNA side of the dsRNA substrate. A symmetrical internal loop of the present disclosure can be formed by 9 nucleotides on the engineered polynucleotide side of the dsRNA target and 9 nucleotides on the target RNA side of the dsRNA substrate. A symmetrical internal loop of the present disclosure can be formed by 10 nucleotides on the engineered polynucleotide side of the dsRNA target and 10 nucleotides on the target RNA side of the dsRNA substrate.

In an aspect, a double stranded RNA (dsRNA) substrate can be formed upon hybridization of an engineered polynucleotide of the present disclosure to a target RNA. An internal loop can be a symmetrical internal loop or an asymmetrical internal loop. An asymmetrical internal loop can be formed when a different number of nucleotides can be present on each side of the internal loop. For example, an asymmetrical internal loop in a dsRNA substrate of the present disclosure can have different numbers of nucleotides on the engineered polynucleotide side and the target RNA side of the dsRNA substrate. An asymmetrical internal loop of the present disclosure can be formed by 5 nucleotides on the engineered polynucleotide side of the dsRNA substrate and 6 nucleotides on the target RNA side of the dsRNA substrate. An asymmetrical internal loop of the present disclosure can be formed by 5 nucleotides on the target RNA side of the dsRNA substrate and 6 nucleotides on the engineered polynucleotide side of the dsRNA substrate. An asymmetrical internal loop of the present disclosure can be formed by 5 nucleotides on the engineered polynucleotide side of the dsRNA substrate and 7 nucleotides on the target RNA side of the dsRNA substrate. An asymmetrical internal loop of the present disclosure can be formed by 5 nucleotides on the target RNA side of the dsRNA substrate and 7 nucleotides on the engineered polynucleotide side of the dsRNA substrate. An asymmetrical internal loop of the present disclosure can be formed by 5 nucleotides on the engineered polynucleotide side of the dsRNA substrate and 8 nucleotides internal loop the target RNA side of the dsRNA substrate. An asymmetrical internal loop of the present disclosure can be formed by 5 nucleotides on the target RNA side of the dsRNA substrate and 8 nucleotides on the engineered polynucleotide side of the dsRNA substrate. An asymmetrical internal loop of the present disclosure can be formed by 5 nucleotides on the engineered polynucleotide side of the dsRNA substrate and 9 nucleotides internal loop the target RNA side of the dsRNA substrate. An asymmetrical internal loop of the present disclosure can be formed by 5 nucleotides on the target RNA side of the dsRNA substrate and 9 nucleotides on the engineered polynucleotide side of the dsRNA substrate. An asymmetrical internal loop of the present disclosure can be formed by 5 nucleotides on the engineered polynucleotide side of the dsRNA substrate and 10 nucleotides internal loop the target RNA side of the dsRNA substrate. An asymmetrical internal loop of the present disclosure can be formed by 5 nucleotides on the target RNA side of the dsRNA substrate and 10 nucleotides on the engineered polynucleotide side of the dsRNA substrate. An asymmetrical internal loop of the present disclosure can be formed by 6 nucleotides on the engineered polynucleotide side of the dsRNA substrate and 7 nucleotides internal loop the target RNA side of the dsRNA substrate. An asymmetrical internal loop of the present disclosure can be formed by 6 nucleotides on the target RNA side of the dsRNA substrate and 7 nucleotides on the engineered polynucleotide side of the dsRNA substrate. An asymmetrical internal loop of the present disclosure can be formed by 6 nucleotides on the engineered polynucleotide side of the dsRNA substrate and 8 nucleotides internal loop the target RNA side of the dsRNA substrate. An asymmetrical internal loop of the present disclosure can be formed by 6 nucleotides on the target RNA side of the dsRNA substrate and 8 nucleotides on the engineered polynucleotide side of the dsRNA substrate. An asymmetrical internal loop of the present disclosure can be formed by 6 nucleotides on the engineered polynucleotide side of the dsRNA substrate and 9 nucleotides internal loop the target RNA side of the dsRNA substrate. An asymmetrical internal loop of the present disclosure can be formed by 6 nucleotides on the target RNA side of the dsRNA substrate and 9 nucleotides on the engineered polynucleotide side of the dsRNA substrate. An asymmetrical internal loop of the present disclosure can be formed by 6 nucleotides on the engineered polynucleotide side of the dsRNA substrate and 10 nucleotides internal loop the target RNA side of the dsRNA substrate. An asymmetrical internal loop of the present disclosure can be formed by 6 nucleotides on the target RNA side of the dsRNA substrate and 10 nucleotides on the engineered polynucleotide side of the dsRNA substrate. An asymmetrical internal loop of the present disclosure can be formed by 7 nucleotides on the engineered polynucleotide side of the dsRNA substrate and 8 nucleotides internal loop the target RNA side of the dsRNA substrate. An asymmetrical internal loop of the present disclosure can be formed by 7 nucleotides on the target RNA side of the dsRNA substrate and 8 nucleotides on the engineered polynucleotide side of the dsRNA substrate. An asymmetrical internal loop of the present disclosure can be formed by 7 nucleotides on the engineered polynucleotide side of the dsRNA substrate and 9 nucleotides internal loop the target RNA side of the dsRNA substrate. An asymmetrical internal loop of the present disclosure can be formed by 7 nucleotides on the target RNA side of the dsRNA substrate and 9 nucleotides on the engineered polynucleotide side of the dsRNA substrate. An asymmetrical internal loop of the present disclosure can be formed by 7 nucleotides on the engineered polynucleotide side of the dsRNA substrate and 10 nucleotides internal loop the target RNA side of the dsRNA substrate. An asymmetrical internal loop of the present disclosure can be formed by 7 nucleotides on the target RNA side of the dsRNA substrate and 10 nucleotides on the engineered polynucleotide side of the dsRNA substrate. An asymmetrical internal loop of the present disclosure can be formed by 8 nucleotides on the engineered polynucleotide side of the dsRNA substrate and 9 nucleotides internal loop the target RNA side of the dsRNA substrate. An asymmetrical internal loop of the present disclosure can be formed by 8 nucleotides on the target RNA side of the dsRNA substrate and 9 nucleotides on the engineered polynucleotide side of the dsRNA substrate. An asymmetrical internal loop of the present disclosure can be formed by 8 nucleotides on the engineered polynucleotide side of the dsRNA substrate and 10 nucleotides internal loop the target RNA side of the dsRNA substrate. An asymmetrical internal loop of the present disclosure can be formed by 8 nucleotides on the target RNA side of the dsRNA substrate and 10 nucleotides on the engineered polynucleotide side of the dsRNA substrate. An asymmetrical internal loop of the present disclosure can be formed by 9 nucleotides on the engineered polynucleotide side of the dsRNA substrate and 10 nucleotides internal loop the target RNA side of the dsRNA substrate. An asymmetrical internal loop of the present disclosure can be formed by 9 nucleotides on the target RNA side of the dsRNA substrate and 10 nucleotides on the engineered polynucleotide side of the dsRNA substrate.

Structural features that comprise a bulge or loop can be of any size. In some cases, a bulge or loop comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases. In some cases, a bulge or loop comprise at least about 1-10, 5-15, 10-20, 15-25, or 20-30 bases in total.

In some cases, a structural feature can be a structured motif. As disclosed herein, a structured motif comprises two or more structural features in a dsRNA substrate. A structured motif can comprise of any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, a structural feature comprises an at least partial circularization of a polynucleotide. In some cases, a polynucleotide provided herein can be circularized or in a circular configuration. In some aspects, an at least partially circular polynucleotide lacks a 5′ hydroxyl or a 3′ hydroxyl.

In some embodiments, an engineered polynucleotide can comprise a backbone comprising a plurality of sugar and phosphate moieties covalently linked together. In some cases, a backbone of an engineered polynucleotide can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered polynucleotide can lack a 5′ reducing hydroxyl, a 3′ reducing hydroxyl, or both, capable of being exposed to a solvent. In some embodiments, a backbone of an engineered polynucleotide can lack a 5′ reducing hydroxyl, a 3′ reducing hydroxyl, or both, capable of being exposed to nucleases. In some embodiments, a backbone of an engineered polynucleotide can lack a 5′ reducing hydroxyl, a 3′ reducing hydroxyl, or both, capable of being exposed to hydrolytic enzymes. In some instances, a backbone of an engineered polynucleotide can be represented as a polynucleotide sequence in a circular 2-dimensional format with one nucleotide after the other. In some instances, a backbone of an engineered polynucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

Subject polynucleotides can comprise modifications. A modification can be a substitution, insertion, deletion, chemical modification, physical modification, stabilization, purification, or any combination thereof. In some cases, a modification is a chemical modification. Suitable chemical modifications comprise any one of: 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

A modification can be made at any location of a polynucleotide. In some cases, a modification is located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to a polynucleotide. In some cases, a modification can be permanent. In other cases, a modification can be transient. In some cases, multiple modifications are made to a polynucleic acid. A polynucleic acid modification may alter physio-chemical properties of a nucleotide, such as their conformation, polarity, hydrophobicity, chemical reactivity, base-pairing interactions, or any combination thereof.

A modification can also be a phosphorothioate substitute. In some cases, a natural phosphodiester bond may be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

An engineered polynucleotide can have any frequency of bases. For example, a polynucleotide can have a percent adenine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent cytosine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent thymine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent guanine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent uracil of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%.

In some cases, an engineered polynucleotide can undergo quality control after a modification. In some cases, quality control may include PAGE, HPLC, MS, or any combination thereof. In some cases, a mass of a polynucleotide can be determined. A mass can be determined by LC-MS assay. A mass can be 30,000 amu, 50,000 amu, 70,000 amu, 90,000 amu, 100,000 amu, 120,000 amu, 150,000 amu, 175,000 amu, 200,000 amu, 250,000 amu, 300,000 amu, 350,000 amu, 400,000 amu, to about 500,000 amu. A mass can be of a sodium salt of a polynucleotide.

In some cases, an endotoxin level of a polynucleotide can be determined. A clinically/therapeutically acceptable level of an endotoxin can be less than 3 EU/mL. A clinically/therapeutically acceptable level of an endotoxin can be less than 10 EU/mL. A clinically/therapeutically acceptable level of an endotoxin can be less than 8 EU/mL. A clinically/therapeutically acceptable level of an endotoxin can be less than 5 EU/mL. A clinically/therapeutically acceptable level of an endotoxin can be less than 4 EU/mL. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases, a polynucleotide can undergo sterility testing. A clinically/therapeutically acceptable level of a sterility testing can be 0 or denoted by no growth on a culture. A clinically/therapeutically acceptable level of a sterility testing can be less than 0.5% growth. A clinically/therapeutically acceptable level of a sterility testing can be less than 1% growth.

In some cases, any one of the polynucleotides that comprise recruiting sequences may also comprise structural features described herein.

Also provided are linear engineered polynucleotides. Linear polynucleotides can substantially lack structural features provided herein. For example, a linear polynucleotide can lack a structural feature or can have less than about 2 structural features or partial structures. A partial structure can comprise a portion of the bases required to achieve a structural feature as described herein.

In other cases, a linear engineered polynucleotide can comprise any one of: 5′ hydroxyl, a 3′ hydroxyl, or both. Any one of these can be capable of being exposed to solvent and maintain linearization.

Compositions and methods provided herein can be utilized to modulate expression of a target. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an engineered polynucleotide described herein can facilitate a knockdown. A knockdown can reduce the expression of a target RNA. In some cases, a knockdown can be accompanied by editing of an mRNA. In some cases, a knockdown can occur with substantially little to no editing of an mRNA. In some instances, a knockdown can occur by targeting an untranslated region of the target RNA, such as a 3′ UTR, a 5′ UTR or both. In some cases, a knockdown can occur by targeting a coding region of the target RNA. In some instances, a knockdown can be mediated by an RNA editing enzyme (e.g. ADAR). In some instances, an RNA editing enzyme can cause a knockdown by hydrolytic deamination of multiple adenosines in an RNA. Hydrolytic deamination of multiple adenosines in an RNA can be referred to as hyper-editing. In some cases, hyper-editing can occur in cis (e.g. in an Alu element) or in trans (e.g. in a target RNA by an engineered polynucleotide).

In an aspect, a subject engineered polynucleotide can be utilized to hyper-edit a target RNA or target RNA region. In some cases, hyper-editing can introduce edits in at least 2 or more nucleotides of a subject target RNA. In some cases, hyper-editing can introduce at least or at most about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or at least or at most about 100 edits in a region of a target RNA. In an embodiment, hyper-editing can occur in an untranslated region, translated region, 3′UTR, 5′UTR, or any combinations herein and thereof. In an embodiment, a target RNA of a subject APP mRNA can be hyper-edited to mutagenize the mRNA thereby generating a modified APP polypeptide that has reduced or eliminated cleavage as compared to an otherwise comparable APP polypeptide that is not modified. In an embodiment, a region of a target mRNA can be mutagenized at the β-Secretase cut site and the residues distal and proximal to it such that the modified polypeptide has reduced or eliminated cleavage by a β-Secretase. In an aspect, the reduced cleavage is at least about or at most about 1-fold, 3-fold, 5-fold, 7-fold, 10-fold, 15-fold, 20-fold, 40-fold, 60-fold, 80-fold, or 100-fold reduced as compared to an otherwise comparable target mRNA that does not under hyper-editing, for example using a subject engineered polypeptide system. In an embodiment, hyper-editing can modulate a driver event in disease by reducing or eliminating a substrate preference of β-Secretases (including but not limited to BACE1); and not majorly modulating APP expression which in turn can enable APP's primary cellular functions to remain largely unaffected. BACE1 substrate preferences are shown below, and also the APP mRNA sequence with putative adenosines that may be edited (singly or in combinations). Examples of this methodology are provided in Example 19 and 20.

In an aspect, a subject engineered polynucleotide can be designed utilizing methods that comprise tiling. For example, an engineered polynucleotide can be selected from a plurality of candidate engineered polynucleotides that have been tiled against a nucleic acid or polypeptide of a subject target RNA. In an embodiment, an engineered polynucleotide can be selected from a group of engineered polynucleotides that have been tiled against a nucleic acid or polypeptide of a subject target RNA, such as APP, SNCA, and/or Tau. In some cases, tiling can comprise tiling engineered polynucleotides across regulatory elements of subject targets. In some cases, tiling can comprise tiling engineered polynucleotides across any one of: a poly(A) tail, a microRNA response element (MRE), an AU-rich element (ARE), 5′UTR, 3′UTR, or any combination thereof of subject target sequence.

In an aspect, engineered polynucleotides that are tiled against a target RNA or nucleic acid can be pooled for use in a method described herein. In some cases, engineered polynucleotides can be pooled for detecting a target in a single assay. The pooling of engineered polynucleotides that are tiled against a single target can enhance the detection of a target RNA using the methods described herein. The tiling for example, can be sequential along the target nucleic acid or target polypeptide. Sometimes, the tiling can be overlapping along the target nucleic acid or target RNA. In some instances, the tiling comprises gaps between the tiled engineered polynucleotide along the target nucleic acid or target RNA. In some instances, the tiling of an engineered polynucleotide can be non-sequential. Often, a method for detecting a target nucleic acid and/or target RNA can comprise contacting a target nucleic acid or target RNA to a pool of engineered polynucleotides and an RNA editing entity and/or nuclease, wherein an engineered polynucleotide of the pool of engineered polynucleotides comprises a targeting sequence to a sequence of a target; and assaying for editing.

In some embodiments, engineered polynucleotides can include cis-regulatory elements. Such cis-regulatory elements can include specific RNA sequence. In some cases, the cis-regulatory elements can regulate the RNA abundance, RNA synthesis, RNA stability, RNA degradation, or RNA localization of engineered polynucleotides. Such cis-regulatory elements can comprise Malat1, Xist, Neat1, or snoRNAs sequence. Malat1 sequence can localize engineered polynucleotides to the nucleus. Malat1, Xist, Neat1, or snoRNAs sequence can also provide a nuclear retention signal for the polynucleotide.

In some cases, engineered polynucleotides can be expressed from a Polymerase I, II, or III promoter. In other cases, the promoter can be a tissue-specific promoter. In some cases, multiple engineered polynucleotides can be expressed by a Polymerase I, II, or III promoter. In some cases, multiple engineered polynucleotides can be expressed a Polymerase I, II, or III promoter from one direction by placing multiple engineered polynucleotides from one side of the Polymerase I, II, or III promoter. In other cases, multiple engineered polynucleotides can be expressed a Polymerase I, II, or III promoter from two directions by placing multiple engineered polynucleotides from both sides of the Polymerase I, II, or III promoter.

In some embodiments, a engineered polynucleotide can have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides. In some embodiments, an engineered polynucleotide can have a length from about: 5-50, 10-50, 20-80, 30-100, 40-130, 50-150, 50-200, 50-300, 100-200, 100-150, 100-250, 150-300, 200-300, 300-400, 250-400, 250-500, 250-450, or 400-500 nucleotides in length.

In some embodiments, a reporter assay can be used to measure the editing efficiency of an engineered polynucleotides or a target RNA sequence. A reporter assay can comprise a reporter. A reporter can be a nucleic acid reporter or a protein reporter. A nucleic acid reporter can be maintained on a plasmid, viral vector, non-viral vector, a linear nucleic acid sequence, a circular nucleic acid, a nucleic acid with a 5′ or a 3′ reducing hydroxyl group. In some embodiments, a reporter can comprise a transcriptional, post-transcriptional, a translational, or a post-translational reporter. In some case, a reporter can comprise a luciferase reporter, fluorescence reporter, drug resistance reporter, cell viability reporter, protein activity reporter, enzymatic activity reporter, polypeptide or protein binding activity reporter, nucleic acid activity reporter, or any combinations described herein and thereof.

In some embodiments, a reporter can comprise two kozak start codons. In some embodiments, a reporter can comprise 0, 1, 2, 3, 4, 5, 6, 7, 8, 9, 10, or more kozak start codons. A start codon can comprise ATG. In some cases, a nucleic acid sequence including but not limited to DNA or RNA sequence can be placed upstream or 5′ of a kozak start codon. In some cases, a nucleic acid sequence including but not limited to DNA or RNA sequence can be placed in-frame and/or downstream or 3′ of a kozak start codon. In some embodiments, a sequence placed in-frame and/or downstream or 3′ of a kozak start codon can be translationally initiated at the kozak start codon. In some cases, a sequenced placed in-frame and downstream or 3′ of a kozak start codon may not be translationally initiated at the kozak start codon. In some embodiments, a reporter can comprise a first nucleic acid sequence placed upstream or 5′ of a first kozak start codon and a second nucleic acid sequence placed in-frame and/or downstream or 3′ of the first kozak start codon. In some embodiments, a reporter can comprise a first nucleic acid sequence placed upstream or 5′ of a first kozak start codon and a second nucleic acid sequence placed in-frame and/or downstream or 3′ of a second kozak start codon. In some cases, a first kozak start codon and a second kozak start codon can be separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nucleotides. In other cases, a first kozak start codon and a second kozak start codon can be separated by 0-10, 9-20, 19-30, 29-40, 39-50, 49-100, 99-150, 149-200, 199-250, 249-300, 299-350, 349-400, 399-450, or 499-500 nucleotides. In some cases, a first sequence placed upstream or 5′ of a first kozak start codon can increase or decrease the translation of a second sequence placed in-frame and downstream or 3′ of a second kozak start codon. In some cases, a first sequence placed downstream or 3′ of a first kozak start codon but before a second kozak start codon can increase or decrease the translation of a second sequence placed in-frame and downstream or 3′ of the second kozak start codon. In some embodiments, the sequence 5′ and 3′ of a kozak codon can comprise the sequence of a genomic locus or RNA. An RNA can comprise a precursor-mRNA, a pre-messenger RNA (mRNA), a messenger RNA (mRNA), a ribosomal RNA, a transfer RNA (tRNA), a long non-coding RNA, a small RNA, a nuclear RNA, a cytoplasmic RNA, a prokaryotic RNA, a synthesized RNA, a purified RNA, a single-stranded RNA, a double-stranded RNA, a mitochondrial RNA, and any combination thereof. In some embodiments, a suitable RNA to target can comprise a ribozyme, isolated RNA of a sequence, sgRNA, guide RNA, snRNA, long non-coding RNA, long intergenic non-coding RNA, enhancer RNA, extracellular RNA, Y RNA, hnRNA, scaRNA, circRNA, snoRNA, siRNA, miRNA, tRNA-derived small RNA (tsRNA), antisense RNA, shRNA, small rDNA-derived RNA (srRNA), a portion thereof, and any combination thereof. In some embodiment, a portion of an RNA can be coding or non-coding. In other cases, a portion of an RNA can comprise a 5′UTR or 3′UTR. In some instances, a portion of an RNA can comprise a sequence from a Charcot-Marie-Tooth Syndrome 1A, APP, tau, or alpha-synuclein mRNA. In some cases, a second sequence placed in-frame and downstream or 3′ of a second kozak start codon can comprise a sequence encoding a reporter or a portion of a reporter described herein and thereof.

In some embodiments, an editing of a base of a nucleotide of a first sequence placed upstream/5′ or downstream of a first kozak start codon can increase or decrease the translation of a second nucleic acid sequence placed in-frame and/or downstream or 3′ of the first kozak start codon. In some cases, an increase or decrease of the translation of a second nucleic acid sequence placed in-frame and/or downstream or 3′ of a first kozak start codon can comprise an increase or decrease in translational initiation, elongation, termination, re-initiation, or any combinations thereof of the second nucleic acid sequence. In some embodiments, an amount of an editing of a base of a nucleotide of a first sequence placed upstream/5′ of a first kozak start codon can be linear to the translational initiation, elongation, termination, re-initiation, or any combinations thereof of a second nucleic acid sequence placed downstream or 3′ of the first kozak codon. In some embodiments, an amount of an editing of a base of a nucleotide of a sequence placed upstream/5′ or downstream/3′ of a first kozak start codon may not be linear to the translational initiation, elongation, termination, re-initiation, or any combinations thereof of a second nucleic acid sequence placed downstream or 3′ of the first kozak codon.

In some embodiments, an editing of a base of a nucleotide of a sequence placed upstream/5′ or downstream/3′ of a first kozak start codon can increase or decrease the translation of a second nucleic acid sequence placed in-frame and/or downstream or 3′ of a second kozak start codon. In some cases, an increase or decrease of the translation of a second nucleic acid sequence placed in-frame and/or downstream or 3′ of a second kozak start codon can comprise an increase or decrease in translational initiation, elongation, termination, re-initiation, or any combinations thereof of the second nucleic acid sequence. In some embodiments, an amount of an editing of a base of a nucleotide of a sequence placed upstream/5′ or downstream/3′ of a first kozak start codon can be linear to the translational initiation, elongation, termination, re-initiation, or any combinations thereof of of a second nucleic acid sequence placed downstream or 3′ of a second kozak codon. In some embodiments, an amount of an editing of a base of a nucleotide of a sequence placed upstream/5′ or downstream/3′ of a first kozak start codon may not be linear to the translational initiation, elongation, termination, re-initiation, or any combinations thereof of a second nucleic acid sequence placed downstream or 3′ of a second kozak codon.

### Suitable Targets

Compositions and methods provided herein can be utilized to target suitable RNA polypeptides and portions thereof. A suitable RNA can comprise a non-protein coding region or a protein coding region. Exemplary non-protein coding regions include but are not limited to a three prime untranslated region (3′UTR), five prime untranslated region (5′UTR), poly(A) tail, a microRNA response element (MRE), AU-rich element (ARE), or any combination thereof. A suitable RNA can also comprise an intron, exon, or any combination thereof. In some cases, the engineered guide RNAs disclosed herein target a non-protein coding region in order to facilitate target protein knockdown.

In some cases, a suitable RNA to target includes but is not limited to: a precursor-mRNA, a pre-messenger RNA (mRNA), a messenger RNA (mRNA), a ribosomal RNA, a transfer RNA (tRNA), a long non-coding RNA, a small RNA, a nuclear RNA, a cytoplasmic RNA, a prokaryotic RNA, a synthesized RNA, a purified RNA, a single-stranded RNA, a double-stranded RNA, a mitochondrial RNA, and any combination thereof. In some embodiments, a suitable RNA to target can comprise a ribozyme, isolated RNA of a sequence, sgRNA, guide RNA, snRNA, long non-coding RNA, long intergenic non-coding RNA, enhancer RNA, extracellular RNA, Y RNA, hnRNA, scaRNA, circRNA, snoRNA, siRNA, miRNA, tRNA-derived small RNA (tsRNA), antisense RNA, shRNA, small rDNA-derived RNA (srRNA), and any combination thereof.

A messenger RNA or mRNA can comprise a nucleic acid molecule that is transcribed from DNA and then processed to remove non-coding sections known as introns. The resulting mRNA can be exported from the nucleus (or another locus where the DNA is present) and translated into a protein. A pre-mRNA can comprise the nucleic acid strand prior to processing to remove non-coding sections.

Exemplary endogenous targets can also comprise amyloid precursor protein (APP), Tau, and Alpha-synuclein (SNCA).

In some embodiments, the engineered polynucleotides disclosed herein can target a secretase enzyme cleavage site in APP and edit said cleavage site in order to modulate processing and cleavage of APP by secretase enzymes (e.g., a beta secretase such as BACE1, cathepsin B or Meprin beta). In some embodiments, the engineered polynucleotides can modulate the expression of APP. In some cases, the engineered polynucleotides can modulate the transcription or post-transcriptional regulation of the APP mRNA or pre-mRNA. In other cases, the engineered polynucleotides can correct aberrant expression of splice variants generated by a mutation in APP. In some cases, , the engineered polynucleotides can modulate the gene or protein translation of APP. In some embodiments, the engineered polynucleotides can decrease, down-regulate, or knock down the expression of APP by decreasing the abundance of the APP transcript. In some instances, the engineered polynucleotides can decrease or down-regulate the processing, splicing, turnover or stability of the APP transcript; or the accessibility of the APP transcript by translational machinery such as ribosome. In some cases, an engineered polynucleotide can facilitate a knockdown of APP. A knockdown can reduce the expression of APP. In some cases, a knockdown can be accompanied by editing of the APP mRNA or pre-mRNA. In some cases, a knockdown can occur with substantially little to no editing of the APP mRNA or pre-mRNA. In some instances, a knockdown can occur by targeting an untranslated region of the APP mRNA or pre-mRNA, such as a 3′ UTR, a 5′ UTR or both. In some cases, a knockdown can occur by targeting a coding region of the APP mRNA or pre-mRNA.

Compositions described herein can edit the cleavage site in APP, so that β/γ secretases exhibit reduced cleavage of APP or can no longer cut APP, and therefore reduced levels of Abeta 40/Abeta 42 or no Abetas can be produced. Compositions consistent with the present disclosure may combine compositions for target APP cleavage site editing with compositions for Tau (e.g., a microtubule-associated protein Tau (MAPT) encoded from a MAPT gene) knockdown or compositions for Alpha-synuclein (SNCA) knockdown and can have synergistic effects to prevent and/or cure a neurodegenerative disease. The compositions and methods disclosed herein can yield results in editing and/or knockdown of targets without any of the resulting issues seen in small molecule or antibody therapy. Compositions can knockdown APP (instead of target cleavage site editing). Editing at the target cleavage site in APP and knockdown can be deployed singly or in combination.

In some cases, a targeting sequence of an engineered polynucleotide provided herein can at least partially hybridize to a region of a target RNA. A region of a target RNA can comprise: (a) a sequence that at least partially encodes for a suitable target provided herein, (b) a sequence that is proximal to a sequence that at least partially encodes for a suitable target provided herein, (c) comprises (a) and (b). For example, a region of a target RNA can comprise (a) a sequence that at least partially encodes for an APP, (b) a sequence that is proximal to a sequence that at least partially encodes for an APP, or (c) comprises (a) and (b). Other suitable targets can be targeted with engineered polynucleotides disclosed herein.

### Amyloid Precursor Protein (APP)

Pathogenic cleavage of amyloid precursor protein (APP) can create Amyloid beta (Abeta) fragments, which has been implicated in Alzheimer's disease. The accumulation of Abeta fragments can: impair synaptic functions and related signaling pathways, change neuronal activities, trigger the release of neurotoxic mediators from glial cells, or any combination thereof. Abeta can alter kinase function, leading to Tau hyperphosphorylation.

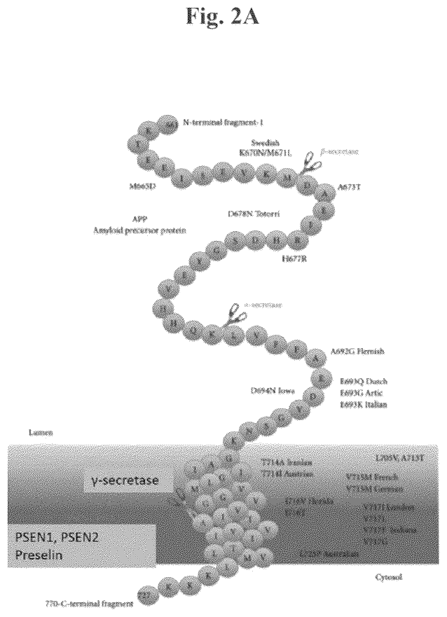

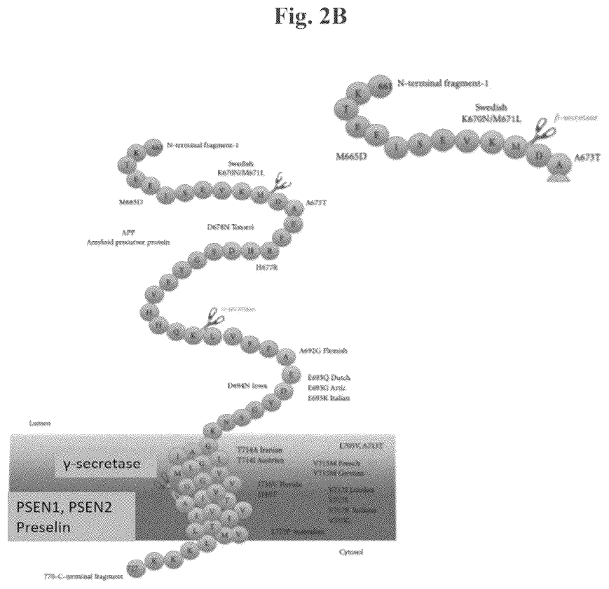

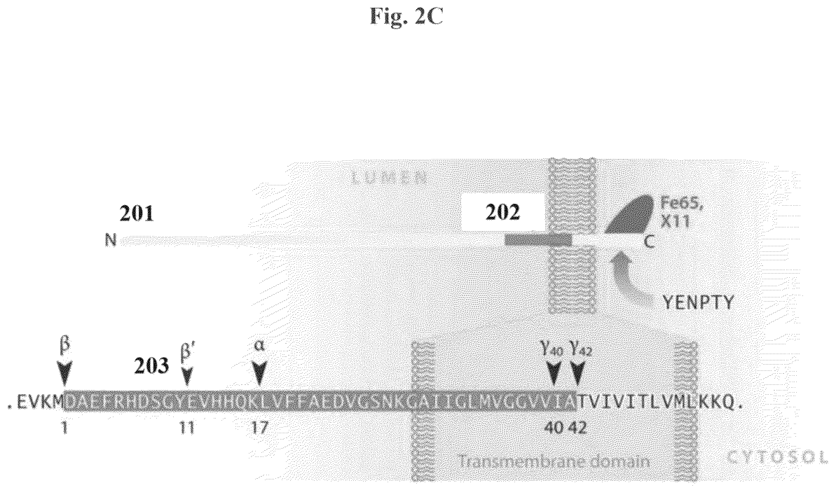

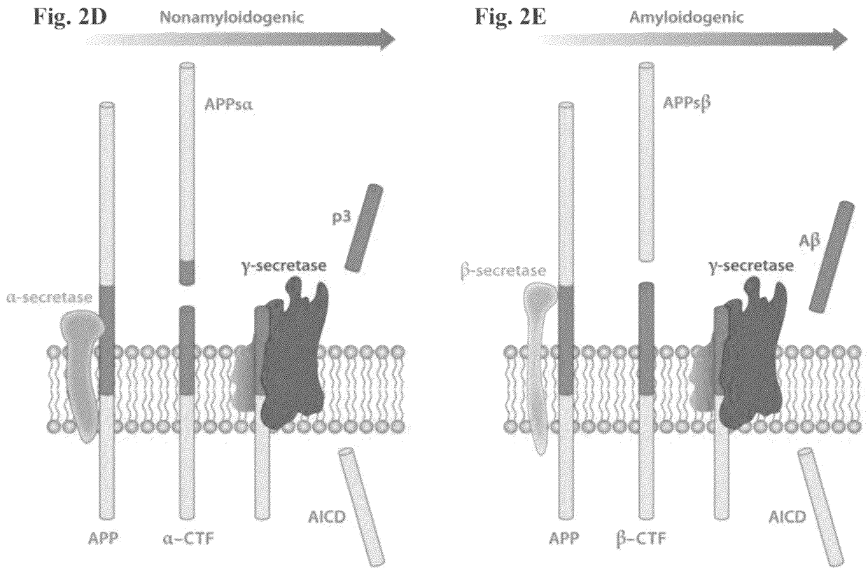

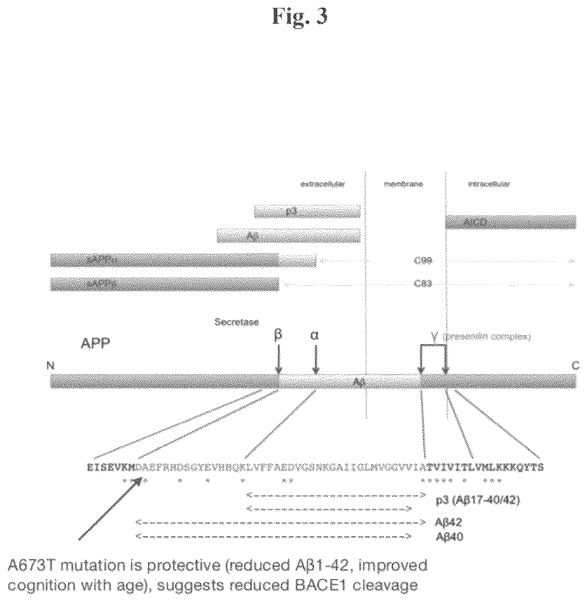

The generation of Abeta by enzymatic cleavages of the β-amyloid precursor protein (APP) is an important player in Alzheimer's disease. As shown in FIGS. 2D, 2E, and 3, the non-amyloidogenic APP processing pathway involves cleavages by alpha- and gamma-secretase. The cleavage by alpha-secretase generates a long form of secreted APP (APPs alpha) and a C-terminal fragment (alpha-CTF). Further processing of alpha-CTF by gamma-secretase generates a p3 and AICD fragment. The amyloidogenic APP processing pathway instead involves cleavages by beta- and gamma-secretase. The cleavage by beta-secretase generates a short form of secreted APP (APPs beta) and a C-terminal fragment (beta-CTF). Further processing of beta-CTF by gamma-secretase generates an Abeta and AICD fragment. The oligomerization and fibrillization of Abeta fragments lead to AD pathology.

FIGS. 2D and 2E was adapted from Thinakaran G, Koo EH. 2008 Amyloid precursor protein trafficking, processing, and function. J. Biol. Chem. 283:29615-19. Herein the methods of which are incorporated by reference.

In some cases, amyloid precursor protein (APP) can be cut by a beta secretase (e.g., BACE1, cathepsin B or Meprin beta) or gamma secretase, and the fragment resulting from such cuts can be Abeta peptides of 36-43 amino acids. Certain Abeta peptide metabolites of this cleavage can be crucially involved in Alzheimer's disease pathology and progression. The wild type sequence of the APP polypeptide is listed in SEQ ID NO: 2.

APP
SEQ ID NO: 2
MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN

The mRNA sequences of human APP are listed in TABLE 1.

TABLE 1
Human APP mRNA Isoform Sequences. Sequences
obtained from NCBI APP gene ID: 381; Assembly
GRCh38.p13 (GCF_000001405.39); NC_000021.9
(25880550 . . . 26171128, complement)

| SEQ ID | Iso- | |
|---|---|---|
| NO | form | mRNA Sequence |
| 3 | 1 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | AGGUGUGCUCUGAACAAGCCGAGACGGGGCCGUGC |
| | | CGAGCAAUGAUCUCCCGCUGGUACUUUGAUGUGAC |
| | | UGAAGGGAAGUGUGCCCCAUUCUUUUACGGCGGAU |
| | | GUGGCGGCAACCGGAACAACUUUGACACAGAAGAG |
| | | UACUGCAUGGCCGUGUGUGGCAGCGCCAUGUCCCA |
| | | AAGUUUACUCAAGACUACCCAGGAACCUCUUGCCC |
| | | GAGAUCCUGUUAAACUUCCUACAACAGCAGCCAGU |
| | | ACCCCUGAUGCCGUUGACAAGUAUCUCGAGACACC |
| | | UGGGGAUGAGAAUGAACAUGCCCAUUUCCAGAAAG |
| | | CCAAAGAGAGGCUUGAGGCCAAGCACCGAGAGAGA |
| | | AUGUCCCAGGUCAUGAGAGAAUGGGAAGAGGCAGA |
| | | ACGUCAAGCAAAGAACUUGCCUAAAGCUGAUAAGA |
| | | AGGCAGUUAUCCAGCAUUUCCAGGAGAAAGUGGAA |
| | | UCUUUGGAACAGGAAGCAGCCAACGAGAGACAGCA |
| | | GCUGGUGGAGACACACAUGGCCAGAGUGGAAGCCA |
| | | UGCUCAAUGACCGCCGCCGCCUGGCCCUGGAGAAC |
| | | UACAUCACCGCUCUGCAGGCUGUUCCUCCUCGGCC |
| | | UCGUCACGUGUUCAAUAUGCUAAAGAAGUAUGUCC |
| | | GCGCAGAACAGAAGGACAGACAGCACACCCUAAAG |
| | | CAUUUCGAGCAUGUGCGCAUGGUGGAUCCCAAGAA |
| | | AGCCGCUCAGAUCCGGUCCCAGGUUAUGACACACC |
| | | UCCGUGUGAUUUAUGAGCGCAUGAAUCAGUCUCUC |
| | | UCCCUGCUCUACAACGUGCCUGCAGUGGCCGAGGA |
| | | GAUUCAGGAUGAAGUUGAUGAGCUGCUUCAGAAAG |
| | | AGCAAAACUAUUCAGAUGACGUCUUGGCCAACAUG |
| | | AUUAGUGAACCAAGGAUCAGUUACGGAAACGAUGC |
| | | UCUCAUGCCAUCUUUGACCGAAACGAAAACCACCG |
| | | UGGAGCUCCUUCCCGUGAAUGGAGAGUUCAGCCUG |
| | | GACGAUCUCCAGCCGUGGCAUUCUUUUGGGGCUGA |
| | | CUCUGUGCCAGCCAACACAGAAAACGAAGUUGAGC |
| | | CUGUUGAUGCCCGCCCUGCUGCCGACCGAGGACUG |
| | | ACCACUCGACCAGGUUCUGGGUUGACAAAUAUCAA |
| | | GACGGAGGAGAUCUCUGAAGUGAAGAUGGAUGCAG |
| | | AAUUCCGACAUGACUCAGGAUAUGAAGUUCAUCAU |
| | | CAAAAAUUGGUGUUCUUUGCAGAAGAUGUGGGUUC |
| | | AAACAAAGGUGCAAUCAUUGGACUCAUGGUGGGCG |
| | | GUGUUGUCAUAGCGACAGUGAUCGUCAUCACCUUG |
| | | GUGAUGCUGAAGAAGAAACAGUACACAUCCAUUCA |
| | | UCAUGGUGUGGUGGAGGUUGACGCCGCUGUCACCC |
| | | CAGAGGAGCGCCACCUGUCCAAGAUGCAGCAGAAC |
| | | GGCUACGAAAAUCCAACCUACAAGUUCUUUGAGCA |
| | | GAUGCAGAACUAGACCCCCGCCACAGCAGCCUCUG |
| | | AAGUUGGACAGCAAAACCAUUGCUUCACUACCCAU |
| | | CGGUGUCCAUUUAUAGAAUAAUGUGGGAAGAAACA |
| | | AACCCGUUUUAUGAUUUACUCAUUAUCGCCUUUUG |
| | | ACAGCUGUGCUGUAACACAAGUAGAUGCCUGAACU |
| | | UGAAUUAAUCCACACAUCAGUAAUGUAUUCUAUCU |
| | | CUCUUUACAUUUUGGUCUCUAUACUACAUUAUUAA |
| | | UGGGUUUUGUGUACUGUAAAGAAUUUAGCUGUAUC |
| | | AAACUAGUGCAUGAAUAGAUUCUCUCCUGAUUAUU |
| | | UAUCACAUAGCCCCUUAGCCAGUUGUAUAUUAUUC |
| | | UUGUGGUUUGUGACCCAAUUAAGUCCUACUUUACA |
| | | UAUGCUUUAAGAAUCGAUGGGGGAUGCUUCAUGUG |
| | | AACGUGGGAGUUCAGCUGCUUCUCUUGCCUAAGUA |
| | | UUCCUUUCCUGAUCACUAUGCAUUUUAAAGUUAAA |
| | | CAUUUUUAAGUAUUUCAGAUGCUUUAGAGAGAUUU |
| | | UUUUUCCAUGACUGCAUUUUACUGUACAGAUUGCU |
| | | GCUUCUGCUAUAUUUGUGAUAUAGGAAUUAAGAGG |
| | | AUACACACGUUUGUUUCUUCGUGCCUGUUUUAUGU |
| | | GCACACAUUAGGCAUUGAGACUUCAAGCUUUUCUU |
| | | UUUUUGUCCACGUAUCUUUGGGUCUUUGAUAAAGA |
| | | AAAGAAUCCCUGUUCAUUGUAAGCACUUUUACGGG |
| | | GCGGGUGGGGAGGGGUGCUCUGCUGGUCUUCAAUU |
| | | ACCAAGAAUUCUCCAAAACAAUUUUCUGCAGGAUG |
| | | AUUGUACAGAAUCAUUGCUUAUGACAUGAUCGCUU |
| | | UCUACACUGUAUUACAUAAAUAAAUUAAAUAAAAU |
| | | AACCCCGGGCAAGACUUUUCUUUGAAGGAUGACUA |
| | | CAGACAUUAAAUAAUCGAAGUAAUUUUGGGUGGGG |
| | | AGAAGAGGCAGAUUCAAUUUUCUUUAACCAGUCUG |
| | | AAGUUUCAUUUAUGAUACAAAAGAAGAUGAAAAUG |
| | | GAAGUGGCAAUAUAAGGGGAUGAGGAAGGCAUGCC |
| | | UGGACAAACCCUUCUUUUAAGAUGUGUCUUCAAUU |
| | | UGUAUAAAAUGGUGUUUUCAUGUAAAUAAAUACAU |
| | | UCUUGGAGGAGCA |
| 4 | 2 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | AGGUGUGCUCUGAACAAGCCGAGACGGGGCCGUGC |
| | | CGAGCAAUGAUCUCCCGCUGGUACUUUGAUGUGAC |
| | | UGAAGGGAAGUGUGCCCCAUUCUUUUACGGCGGAU |
| | | GUGGCGGCAACCGGAACAACUUUGACACAGAAGAG |
| | | UACUGCAUGGCCGUGUGUGGCAGCGCCAUUCCUAC |
| | | AACAGCAGCCAGUACCCCUGAUGCCGUUGACAAGU |
| | | AUCUCGAGACACCUGGGGAUGAGAAUGAACAUGCC |
| | | CAUUUCCAGAAAGCCAAAGAGAGGCUUGAGGCCAA |
| | | GCACCGAGAGAGAAUGUCCCAGGUCAUGAGAGAAU |
| | | GGGAAGAGGCAGAACGUCAAGCAAAGAACUUGCCU |
| | | AAAGCUGAUAAGAAGGCAGUUAUCCAGCAUUUCCA |
| | | GGAGAAAGUGGAAUCUUUGGAACAGGAAGCAGCCA |
| | | ACGAGAGACAGCAGCUGGUGGAGACACACAUGGCC |
| | | AGAGUGGAAGCCAUGCUCAAUGACCGCCGCCGCCU |
| | | GGCCCUGGAGAACUACAUCACCGCUCUGCAGGCUG |
| | | UUCCUCCUCGGCCUCGUCACGUGUUCAAUAUGCUA |
| | | AAGAAGUAUGUCCGCGCAGAACAGAAGGACAGACA |
| | | GCACACCCUAAAGCAUUUCGAGCAUGUGCGCAUGG |
| | | UGGAUCCCAAGAAAGCCGCUCAGAUCCGGUCCCAG |
| | | GUUAUGACACACCUCCGUGUGAUUUAUGAGCGCAU |
| | | GAAUCAGUCUCUCUCCCUGCUCUACAACGUGCCUG |
| | | CAGUGGCCGAGGAGAUUCAGGAUGAAGUUGAUGAG |
| | | CUGCUUCAGAAAGAGCAAAACUAUUCAGAUGACGU |
| | | CUUGGCCAACAUGAUUAGUGAACCAAGGAUCAGUU |
| | | ACGGAAACGAUGCUCUCAUGCCAUCUUUGACCGAA |
| | | ACGAAAACCACCGUGGAGCUCCUUCCCGUGAAUGG |
| | | AGAGUUCAGCCUGGACGAUCUCCAGCCGUGGCAUU |
| | | CUUUUGGGGCUGACUCUGUGCCAGCCAACACAGAA |
| | | AACGAAGUUGAGCCUGUUGAUGCCCGCCCUGCUGC |
| | | CGACCGAGGACUGACCACUCGACCAGGUUCUGGGU |
| | | UGACAAAUAUCAAGACGGAGGAGAUCUCUGAAGUG |
| | | AAGAUGGAUGCAGAAUUCCGACAUGACUCAGGAUA |
| | | UGAAGUUCAUCAUCAAAAAUUGGUGUUCUUUGCAG |
| | | AAGAUGUGGGUUCAAACAAAGGUGCAAUCAUUGGA |
| | | CUCAUGGUGGGCGGUGUUGUCAUAGCGACAGUGAU |
| | | CGUCAUCACCUUGGUGAUGCUGAAGAAGAAACAGU |
| | | ACACAUCCAUUCAUCAUGGUGUGGUGGAGGUUGAC |
| | | GCCGCUGUCACCCCAGAGGAGCGCCACCUGUCCAA |
| | | GAUGCAGCAGAACGGCUACGAAAAUCCAACCUACA |
| | | AGUUCUUUGAGCAGAUGCAGAACUAGACCCCCGCC |
| | | ACAGCAGCCUCUGAAGUUGGACAGCAAAACCAUUG |
| | | CUUCACUACCCAUCGGUGUCCAUUUAUAGAAUAAU |
| | | GUGGGAAGAAACAAACCCGUUUUAUGAUUUACUCA |
| | | UUAUCGCCUUUUGACAGCUGUGCUGUAACACAAGU |
| | | AGAUGCCUGAACUUGAAUUAAUCCACACAUCAGUA |
| | | AUGUAUUCUAUCUCUCUUUACAUUUUGGUCUCUAU |
| | | ACUACAUUAUUAAUGGGUUUUGUGUACUGUAAAGA |
| | | AUUUAGCUGUAUCAAACUAGUGCAUGAAUAGAUUC |
| | | UCUCCUGAUUAUUUAUCACAUAGCCCCUUAGCCAG |
| | | UUGUAUAUUAUUCUUGUGGUUUGUGACCCAAUUAA |
| | | GUCCUACUUUACAUAUGCUUUAAGAAUCGAUGGGG |
| | | GAUGCUUCAUGUGAACGUGGGAGUUCAGCUGCUUC |
| | | UCUUGCCUAAGUAUUCCUUUCCUGAUCACUAUGCA |
| | | UUUUAAAGUUAAACAUUUUUAAGUAUUUCAGAUGC |
| | | UUUAGAGAGAUUUUUUUUCCAUGACUGCAUUUUAC |
| | | UGUACAGAUUGCUGCUUCUGCUAUAUUUGUGAUAU |
| | | AGGAAUUAAGAGGAUACACACGUUUGUUUCUUCGU |
| | | GCCUGUUUUAUGUGCACACAUUAGGCAUUGAGACU |
| | | UCAAGCUUUUCUUUUUUUGUCCACGUAUCUUUGGG |
| | | UCUUUGAUAAAGAAAAGAAUCCCUGUUCAUUGUAA |
| | | GCACUUUUACGGGGCGGGUGGGGAGGGGUGCUCUG |
| | | CUGGUCUUCAAUUACCAAGAAUUCUCCAAAACAAU |
| | | UUUCUGCAGGAUGAUUGUACAGAAUCAUUGCUUAU |
| | | GACAUGAUCGCUUUCUACACUGUAUUACAUAAAUA |
| | | AAUUAAAUAAAAUAACCCCGGGCAAGACUUUUCUU |
| | | UGAAGGAUGACUACAGACAUUAAAUAAUCGAAGUA |
| | | AUUUUGGGUGGGGAGAAGAGGCAGAUUCAAUUUUC |
| | | UUUAACCAGUCUGAAGUUUCAUUUAUGAUACAAAA |
| | | GAAGAUGAAAAUGGAAGUGGCAAUAUAAGGGGAUG |
| | | AGGAAGGCAUGCCUGGACAAACCCUUCUUUUAAGA |
| | | UGUGUCUUCAAUUUGUAUAAAAUGGUGUUUUCAUG |
| | | UAAAUAAAUACAUUCUUGGAGGAGCA |
| 5 | 3 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | UUCCUACAACAGCAGCCAGUACCCCUGAUGCCGUU |
| | | GACAAGUAUCUCGAGACACCUGGGGAUGAGAAUGA |
| | | ACAUGCCCAUUUCCAGAAAGCCAAAGAGAGGCUUG |
| | | AGGCCAAGCACCGAGAGAGAAUGUCCCAGGUCAUG |
| | | AGAGAAUGGGAAGAGGCAGAACGUCAAGCAAAGAA |
| | | CUUGCCUAAAGCUGAUAAGAAGGCAGUUAUCCAGC |
| | | AUUUCCAGGAGAAAGUGGAAUCUUUGGAACAGGAA |
| | | GCAGCCAACGAGAGACAGCAGCUGGUGGAGACACA |
| | | CAUGGCCAGAGUGGAAGCCAUGCUCAAUGACCGCC |
| | | GCCGCCUGGCCCUGGAGAACUACAUCACCGCUCUG |
| | | CAGGCUGUUCCUCCUCGGCCUCGUCACGUGUUCAA |
| | | UAUGCUAAAGAAGUAUGUCCGCGCAGAACAGAAGG |
| | | ACAGACAGCACACCCUAAAGCAUUUCGAGCAUGUG |
| | | CGCAUGGUGGAUCCCAAGAAAGCCGCUCAGAUCCG |
| | | GUCCCAGGUUAUGACACACCUCCGUGUGAUUUAUG |
| | | AGCGCAUGAAUCAGUCUCUCUCCCUGCUCUACAAC |
| | | GUGCCUGCAGUGGCCGAGGAGAUUCAGGAUGAAGU |
| | | UGAUGAGCUGCUUCAGAAAGAGCAAAACUAUUCAG |
| | | AUGACGUCUUGGCCAACAUGAUUAGUGAACCAAGG |
| | | AUCAGUUACGGAAACGAUGCUCUCAUGCCAUCUUU |
| | | GACCGAAACGAAAACCACCGUGGAGCUCCUUCCCG |
| | | UGAAUGGAGAGUUCAGCCUGGACGAUCUCCAGCCG |
| | | UGGCAUUCUUUUGGGGCUGACUCUGUGCCAGCCAA |
| | | CACAGAAAACGAAGUUGAGCCUGUUGAUGCCCGCC |
| | | CUGCUGCCGACCGAGGACUGACCACUCGACCAGGU |
| | | UCUGGGUUGACAAAUAUCAAGACGGAGGAGAUCUC |
| | | UGAAGUGAAGAUGGAUGCAGAAUUCCGACAUGACU |
| | | CAGGAUAUGAAGUUCAUCAUCAAAAAUUGGUGUUC |
| | | UUUGCAGAAGAUGUGGGUUCAAACAAAGGUGCAAU |
| | | CAUUGGACUCAUGGUGGGCGGUGUUGUCAUAGCGA |
| | | CAGUGAUCGUCAUCACCUUGGUGAUGCUGAAGAAG |
| | | AAACAGUACACAUCCAUUCAUCAUGGUGUGGUGGA |
| | | GGUUGACGCCGCUGUCACCCCAGAGGAGCGCCACC |
| | | UGUCCAAGAUGCAGCAGAACGGCUACGAAAAUCCA |
| | | ACCUACAAGUUCUUUGAGCAGAUGCAGAACUAGAC |
| | | CCCCGCCACAGCAGCCUCUGAAGUUGGACAGCAAA |
| | | ACCAUUGCUUCACUACCCAUCGGUGUCCAUUUAUA |
| | | GAAUAAUGUGGGAAGAAACAAACCCGUUUUAUGAU |
| | | UUACUCAUUAUCGCCUUUUGACAGCUGUGCUGUAA |
| | | CACAAGUAGAUGCCUGAACUUGAAUUAAUCCACAC |
| | | AUCAGUAAUGUAUUCUAUCUCUCUUUACAUUUUGG |
| | | UCUCUAUACUACAUUAUUAAUGGGUUUUGUGUACU |
| | | GUAAAGAAUUUAGCUGUAUCAAACUAGUGCAUGAA |
| | | UAGAUUCUCUCCUGAUUAUUUAUCACAUAGCCCCU |
| | | UAGCCAGUUGUAUAUUAUUCUUGUGGUUUGUGACC |
| | | CAAUUAAGUCCUACUUUACAUAUGCUUUAAGAAUC |
| | | GAUGGGGGAUGCUUCAUGUGAACGUGGGAGUUCAG |
| | | CUGCUUCUCUUGCCUAAGUAUUCCUUUCCUGAUCA |
| | | CUAUGCAUUUUAAAGUUAAACAUUUUUAAGUAUUU |
| | | CAGAUGCUUUAGAGAGAUUUUUUUUCCAUGACUGC |
| | | AUUUUACUGUACAGAUUGCUGCUUCUGCUAUAUUU |
| | | GUGAUAUAGGAAUUAAGAGGAUACACACGUUUGUU |
| | | UCUUCGUGCCUGUUUUAUGUGCACACAUUAGGCAU |
| | | UGAGACUUCAAGCUUUUCUUUUUUUGUCCACGUAU |
| | | CUUUGGGUCUUUGAUAAAGAAAAGAAUCCCUGUUC |
| | | AUUGUAAGCACUUUUACGGGGCGGGUGGGGAGGGG |
| | | UGCUCUGCUGGUCUUCAAUUACCAAGAAUUCUCCA |
| | | AAACAAUUUUCUGCAGGAUGAUUGUACAGAAUCAU |
| | | UGCUUAUGACAUGAUCGCUUUCUACACUGUAUUAC |
| | | AUAAAUAAAUUAAAUAAAAUAACCCCGGGCAAGAC |
| | | UUUUCUUUGAAGGAUGACUACAGACAUUAAAUAAU |
| | | CGAAGUAAUUUUGGGUGGGGAGAAGAGGCAGAUUC |
| | | AAUUUUCUUUAACCAGUCUGAAGUUUCAUUUAUGA |
| | | UACAAAAGAAGAUGAAAAUGGAAGUGGCAAUAUAA |
| | | GGGGAUGAGGAAGGCAUGCCUGGACAAACCCUUCU |
| | | UUUAAGAUGUGUCUUCAAUUUGUAUAAAAUGGUGU |
| | | UUUCAUGUAAAUAAAUACAUUCUUGGAGGAGCA |
| 6 | 4 | AAAUAGCACAGCCUUGCUGUGCGUGGUAGAAGUUG |
| | | GGUUAGUGUUGACAUGCUGUUGACUCACCCUCCCG |
| | | AGGAUGGAAGCUCUGGCCUGGGUCAAGUUGUGGUC |
| | | ACUGCAGUUAACAGUUUGUUGAUCUCAGGGAGUAU |
| | | UCCACAGUUGCUGAUGUAAUUGACAAUGAUUGGAG |
| | | CCAGCUCUUCCCCAGAUUCAAAUGGACCAAUUAGA |
| | | GGACUUGUUGGUUCUGUUUAUCAACUAUGUACCCA |
| | | CUGAUGGUAAUGCUGGCCUGCUGGCUGAACCCCAG |
| | | AUUGCCAUGUUCUGUGGCAGACUGAACAUGCACAU |
| | | GAAUGUCCAGAAUGGGAAGUGGGAUUCAGAUCCAU |
| | | CAGGGACCAAAACCUGCAUUGAUACCAAGGAAGGC |
| | | AUCCUGCAGUAUUGCCAAGAAGUCUACCCUGAACU |
| | | GCAGAUCACCAAUGUGGUAGAAGCCAACCAACCAG |
| | | UGACCAUCCAGAACUGGUGCAAGCGGGGCCGCAAG |
| | | CAGUGCAAGACCCAUCCCCACUUUGUGAUUCCCUA |
| | | CCGCUGCUUAGUUGGUGAGUUUGUAAGUGAUGCCC |
| | | UUCUCGUUCCUGACAAGUGCAAAUUCUUACACCAG |
| | | GAGAGGAUGGAUGUUUGCGAAACUCAUCUUCACUG |
| | | GCACACCGUCGCCAAAGAGACAUGCAGUGAGAAGA |
| | | GUACCAACUUGCAUGACUACGGCAUGUUGCUGCCC |
| | | UGCGGAAUUGACAAGUUCCGAGGGGUAGAGUUUGU |
| | | GUGUUGCCCACUGGCUGAAGAAAGUGACAAUGUGG |
| | | AUUCUGCUGAUGCGGAGGAGGAUGACUCGGAUGUC |
| | | UGGUGGGGCGGAGCAGACACAGACUAUGCAGAUGG |
| | | GAGUGAAGACAAAGUAGUAGAAGUAGCAGAGGAGG |
| | | AAGAAGUGGCUGAGGUGGAAGAAGAAGAAGCCGAU |
| | | GAUGACGAGGACGAUGAGGAUGGUGAUGAGGUAGA |
| | | GGAAGAGGCUGAGGAACCCUACGAAGAAGCCACAG |
| | | AGAGAACCACCAGCAUUGCCACCACCACCACCACC |
| | | ACCACAGAGUCUGUGGAAGAGGUGGUUCGAGAGGU |
| | | GUGCUCUGAACAAGCCGAGACGGGGCCGUGCCGAG |
| | | CAAUGAUCUCCCGCUGGUACUUUGAUGUGACUGAA |
| | | GGGAAGUGUGCCCCAUUCUUUUACGGCGGAUGUGG |
| | | CGGCAACCGGAACAACUUUGACACAGAAGAGUACU |
| | | GCAUGGCCGUGUGUGGCAGCGCCAUUCCUACAACA |
| | | GCAGCCAGUACCCCUGAUGCCGUUGACAAGUAUCU |
| | | CGAGACACCUGGGGAUGAGAAUGAACAUGCCCAUU |
| | | UCCAGAAAGCCAAAGAGAGGCUUGAGGCCAAGCAC |
| | | CGAGAGAGAAUGUCCCAGGUCAUGAGAGAAUGGGA |
| | | AGAGGCAGAACGUCAAGCAAAGAACUUGCCUAAAG |
| | | CUGAUAAGAAGGCAGUUAUCCAGCAUUUCCAGGAG |
| | | AAAGUGGAAUCUUUGGAACAGGAAGCAGCCAACGA |
| | | GAGACAGCAGCUGGUGGAGACACACAUGGCCAGAG |
| | | UGGAAGCCAUGCUCAAUGACCGCCGCCGCCUGGCC |
| | | CUGGAGAACUACAUCACCGCUCUGCAGGCUGUUCC |
| | | UCCUCGGCCUCGUCACGUGUUCAAUAUGCUAAAGA |
| | | AGUAUGUCCGCGCAGAACAGAAGGACAGACAGCAC |
| | | ACCCUAAAGCAUUUCGAGCAUGUGCGCAUGGUGGA |
| | | UCCCAAGAAAGCCGCUCAGAUCCGGUCCCAGGUUA |
| | | UGACACACCUCCGUGUGAUUUAUGAGCGCAUGAAU |
| | | CAGUCUCUCUCCCUGCUCUACAACGUGCCUGCAGU |
| | | GGCCGAGGAGAUUCAGGAUGAAGUUGAUGAGCUGC |
| | | UUCAGAAAGAGCAAAACUAUUCAGAUGACGUCUUG |
| | | GCCAACAUGAUUAGUGAACCAAGGAUCAGUUACGG |
| | | AAACGAUGCUCUCAUGCCAUCUUUGACCGAAACGA |
| | | AAACCACCGUGGAGCUCCUUCCCGUGAAUGGAGAG |
| | | UUCAGCCUGGACGAUCUCCAGCCGUGGCAUUCUUU |
| | | UGGGGCUGACUCUGUGCCAGCCAACACAGAAAACG |
| | | AAGUUGAGCCUGUUGAUGCCCGCCCUGCUGCCGAC |
| | | CGAGGACUGACCACUCGACCAGGUUCUGGGUUGAC |
| | | AAAUAUCAAGACGGAGGAGAUCUCUGAAGUGAAGA |
| | | UGGAUGCAGAAUUCCGACAUGACUCAGGAUAUGAA |
| | | GUUCAUCAUCAAAAAUUGGUGUUCUUUGCAGAAGA |
| | | UGUGGGUUCAAACAAAGGUGCAAUCAUUGGACUCA |
| | | UGGUGGGCGGUGUUGUCAUAGCGACAGUGAUCGUC |
| | | AUCACCUUGGUGAUGCUGAAGAAGAAACAGUACAC |
| | | AUCCAUUCAUCAUGGUGUGGUGGAGGUUGACGCCG |
| | | CUGUCACCCCAGAGGAGCGCCACCUGUCCAAGAUG |
| | | CAGCAGAACGGCUACGAAAAUCCAACCUACAAGUU |
| | | CUUUGAGCAGAUGCAGAACUAGACCCCCGCCACAG |
| | | CAGCCUCUGAAGUUGGACAGCAAAACCAUUGCUUC |
| | | ACUACCCAUCGGUGUCCAUUUAUAGAAUAAUGUGG |
| | | GAAGAAACAAACCCGUUUUAUGAUUUACUCAUUAU |
| | | CGCCUUUUGACAGCUGUGCUGUAACACAAGUAGAU |
| | | GCCUGAACUUGAAUUAAUCCACACAUCAGUAAUGU |
| | | AUUCUAUCUCUCUUUACAUUUUGGUCUCUAUACUA |
| | | CAUUAUUAAUGGGUUUUGUGUACUGUAAAGAAUUU |
| | | AGCUGUAUCAAACUAGUGCAUGAAUAGAUUCUCUC |
| | | CUGAUUAUUUAUCACAUAGCCCCUUAGCCAGUUGU |
| | | AUAUUAUUCUUGUGGUUUGUGACCCAAUUAAGUCC |
| | | UACUUUACAUAUGCUUUAAGAAUCGAUGGGGGAUG |
| | | CUUCAUGUGAACGUGGGAGUUCAGCUGCUUCUCUU |
| | | GCCUAAGUAUUCCUUUCCUGAUCACUAUGCAUUUU |
| | | AAAGUUAAACAUUUUUAAGUAUUUCAGAUGCUUUA |
| | | GAGAGAUUUUUUUUCCAUGACUGCAUUUUACUGUA |
| | | CAGAUUGCUGCUUCUGCUAUAUUUGUGAUAUAGGA |
| | | AUUAAGAGGAUACACACGUUUGUUUCUUCGUGCCU |
| | | GUUUUAUGUGCACACAUUAGGCAUUGAGACUUCAA |
| | | GCUUUUCUUUUUUUGUCCACGUAUCUUUGGGUCUU |
| | | UGAUAAAGAAAAGAAUCCCUGUUCAUUGUAAGCAC |
| | | UUUUACGGGGCGGGUGGGGAGGGGUGCUCUGCUGG |
| | | UCUUCAAUUACCAAGAAUUCUCCAAAACAAUUUUC |
| | | UGCAGGAUGAUUGUACAGAAUCAUUGCUUAUGACA |
| | | UGAUCGCUUUCUACACUGUAUUACAUAAAUAAAUU |
| | | AAAUAAAAUAACCCCGGGCAAGACUUUUCUUUGAA |
| | | GGAUGACUACAGACAUUAAAUAAUCGAAGUAAUUU |
| | | UGGGUGGGGAGAAGAGGCAGAUUCAAUUUUCUUUA |
| | | ACCAGUCUGAAGUUUCAUUUAUGAUACAAAAGAAG |
| | | AUGAAAAUGGAAGUGGCAAUAUAAGGGGAUGAGGA |
| | | AGGCAUGCCUGGACAAACCCUUCUUUUAAGAUGUG |
| | | UCUUCAAUUUGUAUAAAAUGGUGUUUUCAUGUAAA |
| | | UAAAUACAUUCUUGGAGGAGCAAAAAAAAAAAAAA |
| | | AA |
| 7 | 5 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUC |
| | | UACCCUGAACUGCAGAUCACCAAUGUGGUAGAAGC |
| | | CAACCAACCAGUGACCAUCCAGAACUGGUGCAAGC |
| | | GGGGCCGCAAGCAGUGCAAGACCCAUCCCCACUUU |
| | | GUGAUUCCCUACCGCUGCUUAGUUGGUGAGUUUGU |
| | | AAGUGAUGCCCUUCUCGUUCCUGACAAGUGCAAAU |
| | | UCUUACACCAGGAGAGGAUGGAUGUUUGCGAAACU |
| | | CAUCUUCACUGGCACACCGUCGCCAAAGAGACAUG |
| | | CAGUGAGAAGAGUACCAACUUGCAUGACUACGGCA |
| | | UGUUGCUGCCCUGCGGAAUUGACAAGUUCCGAGGG |
| | | GUAGAGUUUGUGUGUUGCCCACUGGCUGAAGAAAG |
| | | UGACAAUGUGGAUUCUGCUGAUGCGGAGGAGGAUG |
| | | ACUCGGAUGUCUGGUGGGGCGGAGCAGACACAGAC |
| | | UAUGCAGAUGGGAGUGAAGACAAAGUAGUAGAAGU |
| | | AGCAGAGGAGGAAGAAGUGGCUGAGGUGGAAGAAG |
| | | AAGAAGCCGAUGAUGACGAGGACGAUGAGGAUGGU |
| | | GAUGAGGUAGAGGAAGAGGCUGAGGAACCCUACGA |
| | | AGAAGCCACAGAGAGAACCACCAGCAUUGCCACCA |
| | | CCACCACCACCACCACAGAGUCUGUGGAAGAGGUG |
| | | GUUCGAGUUCCUACAACAGCAGCCAGUACCCCUGA |
| | | UGCCGUUGACAAGUAUCUCGAGACACCUGGGGAUG |
| | | AGAAUGAACAUGCCCAUUUCCAGAAAGCCAAAGAG |
| | | AGGCUUGAGGCCAAGCACCGAGAGAGAAUGUCCCA |
| | | GGUCAUGAGAGAAUGGGAAGAGGCAGAACGUCAAG |
| | | CAAAGAACUUGCCUAAAGCUGAUAAGAAGGCAGUU |
| | | AUCCAGCAUUUCCAGGAGAAAGUGGAAUCUUUGGA |
| | | ACAGGAAGCAGCCAACGAGAGACAGCAGCUGGUGG |
| | | AGACACACAUGGCCAGAGUGGAAGCCAUGCUCAAU |
| | | GACCGCCGCCGCCUGGCCCUGGAGAACUACAUCAC |
| | | CGCUCUGCAGGCUGUUCCUCCUCGGCCUCGUCACG |
| | | UGUUCAAUAUGCUAAAGAAGUAUGUCCGCGCAGAA |
| | | CAGAAGGACAGACAGCACACCCUAAAGCAUUUCGA |
| | | GCAUGUGCGCAUGGUGGAUCCCAAGAAAGCCGCUC |
| | | AGAUCCGGUCCCAGGUUAUGACACACCUCCGUGUG |
| | | AUUUAUGAGCGCAUGAAUCAGUCUCUCUCCCUGCU |
| | | CUACAACGUGCCUGCAGUGGCCGAGGAGAUUCAGG |
| | | AUGAAGUUGAUGAGCUGCUUCAGAAAGAGCAAAAC |
| | | UAUUCAGAUGACGUCUUGGCCAACAUGAUUAGUGA |
| | | ACCAAGGAUCAGUUACGGAAACGAUGCUCUCAUGC |
| | | CAUCUUUGACCGAAACGAAAACCACCGUGGAGCUC |
| | | CUUCCCGUGAAUGGAGAGUUCAGCCUGGACGAUCU |
| | | CCAGCCGUGGCAUUCUUUUGGGGCUGACUCUGUGC |
| | | CAGCCAACACAGAAAACGAAGUUGAGCCUGUUGAU |
| | | GCCCGCCCUGCUGCCGACCGAGGACUGACCACUCG |
| | | ACCAGGUUCUGGGUUGACAAAUAUCAAGACGGAGG |
| | | AGAUCUCUGAAGUGAAGAUGGAUGCAGAAUUCCGA |
| | | CAUGACUCAGGAUAUGAAGUUCAUCAUCAAAAAUU |
| | | GGUGUUCUUUGCAGAAGAUGUGGGUUCAAACAAAG |
| | | GUGCAAUCAUUGGACUCAUGGUGGGCGGUGUUGUC |
| | | AUAGCGACAGUGAUCGUCAUCACCUUGGUGAUGCU |
| | | GAAGAAGAAACAGUACACAUCCAUUCAUCAUGGUG |
| | | UGGUGGAGGUUGACGCCGCUGUCACCCCAGAGGAG |
| | | CGCCACCUGUCCAAGAUGCAGCAGAACGGCUACGA |
| | | AAAUCCAACCUACAAGUUCUUUGAGCAGAUGCAGA |
| | | ACUAGACCCCCGCCACAGCAGCCUCUGAAGUUGGA |
| | | CAGCAAAACCAUUGCUUCACUACCCAUCGGUGUCC |
| | | AUUUAUAGAAUAAUGUGGGAAGAAACAAACCCGUU |
| | | UUAUGAUUUACUCAUUAUCGCCUUUUGACAGCUGU |
| | | GCUGUAACACAAGUAGAUGCCUGAACUUGAAUUAA |
| | | UCCACACAUCAGUAAUGUAUUCUAUCUCUCUUUAC |
| | | AUUUUGGUCUCUAUACUACAUUAUUAAUGGGUUUU |
| | | GUGUACUGUAAAGAAUUUAGCUGUAUCAAACUAGU |
| | | GCAUGAAUAGAUUCUCUCCUGAUUAUUUAUCACAU |
| | | AGCCCCUUAGCCAGUUGUAUAUUAUUCUUGUGGUU |
| | | UGUGACCCAAUUAAGUCCUACUUUACAUAUGCUUU |
| | | AAGAAUCGAUGGGGGAUGCUUCAUGUGAACGUGGG |
| | | AGUUCAGCUGCUUCUCUUGCCUAAGUAUUCCUUUC |
| | | CUGAUCACUAUGCAUUUUAAAGUUAAACAUUUUUA |
| | | AGUAUUUCAGAUGCUUUAGAGAGAUUUUUUUUCCA |
| | | UGACUGCAUUUUACUGUACAGAUUGCUGCUUCUGC |
| | | UAUAUUUGUGAUAUAGGAAUUAAGAGGAUACACAC |
| | | GUUUGUUUCUUCGUGCCUGUUUUAUGUGCACACAU |
| | | UAGGCAUUGAGACUUCAAGCUUUUCUUUUUUUGUC |
| | | CACGUAUCUUUGGGUCUUUGAUAAAGAAAAGAAUC |
| | | CCUGUUCAUUGUAAGCACUUUUACGGGGCGGGUGG |
| | | GGAGGGGUGCUCUGCUGGUCUUCAAUUACCAAGAA |
| | | UUCUCCAAAACAAUUUUCUGCAGGAUGAUUGUACA |
| | | GAAUCAUUGCUUAUGACAUGAUCGCUUUCUACACU |
| | | GUAUUACAUAAAUAAAUUAAAUAAAAUAACCCCGG |
| | | GCAAGACUUUUCUUUGAAGGAUGACUACAGACAUU |
| | | AAAUAAUCGAAGUAAUUUUGGGUGGGGAGAAGAGG |
| | | CAGAUUCAAUUUUCUUUAACCAGUCUGAAGUUUCA |
| | | UUUAUGAUACAAAAGAAGAUGAAAAUGGAAGUGGC |
| | | AAUAUAAGGGGAUGAGGAAGGCAUGCCUGGACAAA |
| | | CCCUUCUUUUAAGAUGUGUCUUCAAUUUGUAUAAA |
| | | AUGGUGUUUUCAUGUAAAUAAAUACAUUCUUGGAG |
| | | GAGCA |
| 8 | 6 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUC |
| | | UACCCUGAACUGCAGAUCACCAAUGUGGUAGAAGC |
| | | CAACCAACCAGUGACCAUCCAGAACUGGUGCAAGC |
| | | GGGGCCGCAAGCAGUGCAAGACCCAUCCCCACUUU |
| | | GUGAUUCCCUACCGCUGCUUAGUUGGUGAGUUUGU |
| | | AAGUGAUGCCCUUCUCGUUCCUGACAAGUGCAAAU |
| | | UCUUACACCAGGAGAGGAUGGAUGUUUGCGAAACU |
| | | CAUCUUCACUGGCACACCGUCGCCAAAGAGACAUG |
| | | CAGUGAGAAGAGUACCAACUUGCAUGACUACGGCA |
| | | UGUUGCUGCCCUGCGGAAUUGACAAGUUCCGAGGG |
| | | GUAGAGUUUGUGUGUUGCCCACUGGCUGAAGAAAG |
| | | UGACAAUGUGGAUUCUGCUGAUGCGGAGGAGGAUG |
| | | ACUCGGAUGUCUGGUGGGGCGGAGCAGACACAGAC |
| | | UAUGCAGAUGGGAGUGAAGACAAAGUAGUAGAAGU |
| | | AGCAGAGGAGGAAGAAGUGGCUGAGGUGGAAGAAG |
| | | AAGAAGCCGAUGAUGACGAGGACGAUGAGGAUGGU |
| | | GAUGAGGUAGAGGAAGAGGCUGAGGAACCCUACGA |
| | | AGAAGCCACAGAGAGAACCACCAGCAUUGCCACCA |
| | | CCACCACCACCACCACAGAGUCUGUGGAAGAGGUG |
| | | GUUCGAGAGGUGUGCUCUGAACAAGCCGAGACGGG |
| | | GCCGUGCCGAGCAAUGAUCUCCCGCUGGUACUUUG |
| | | AUGUGACUGAAGGGAAGUGUGCCCCAUUCUUUUAC |
| | | GGCGGAUGUGGCGGCAACCGGAACAACUUUGACAC |
| | | AGAAGAGUACUGCAUGGCCGUGUGUGGCAGCGCCA |
| | | UGUCCCAAAGUUUACUCAAGACUACCCAGGAACCU |
| | | CUUGCCCGAGAUCCUGUUAAACUUCCUACAACAGC |
| | | AGCCAGUACCCCUGAUGCCGUUGACAAGUAUCUCG |
| | | AGACACCUGGGGAUGAGAAUGAACAUGCCCAUUUC |
| | | CAGAAAGCCAAAGAGAGGCUUGAGGCCAAGCACCG |
| | | AGAGAGAAUGUCCCAGGUCAUGAGAGAAUGGGAAG |
| | | AGGCAGAACGUCAAGCAAAGAACUUGCCUAAAGCU |
| | | GAUAAGAAGGCAGUUAUCCAGCAUUUCCAGGAGAA |
| | | AGUGGAAUCUUUGGAACAGGAAGCAGCCAACGAGA |
| | | GACAGCAGCUGGUGGAGACACACAUGGCCAGAGUG |
| | | GAAGCCAUGCUCAAUGACCGCCGCCGCCUGGCCCU |
| | | GGAGAACUACAUCACCGCUCUGCAGGCUGUUCCUC |
| | | CUCGGCCUCGUCACGUGUUCAAUAUGCUAAAGAAG |
| | | UAUGUCCGCGCAGAACAGAAGGACAGACAGCACAC |
| | | CCUAAAGCAUUUCGAGCAUGUGCGCAUGGUGGAUC |
| | | CCAAGAAAGCCGCUCAGAUCCGGUCCCAGGUUAUG |
| | | ACACACCUCCGUGUGAUUUAUGAGCGCAUGAAUCA |
| | | GUCUCUCUCCCUGCUCUACAACGUGCCUGCAGUGG |
| | | CCGAGGAGAUUCAGGAUGAAGUUGAUGAGCUGCUU |
| | | CAGAAAGAGCAAAACUAUUCAGAUGACGUCUUGGC |
| | | CAACAUGAUUAGUGAACCAAGGAUCAGUUACGGAA |
| | | ACGAUGCUCUCAUGCCAUCUUUGACCGAAACGAAA |
| | | ACCACCGUGGAGCUCCUUCCCGUGAAUGGAGAGUU |
| | | CAGCCUGGACGAUCUCCAGCCGUGGCAUUCUUUUG |
| | | GGGCUGACUCUGUGCCAGCCAACACAGAAAACGAA |
| | | GUUGAGCCUGUUGAUGCCCGCCCUGCUGCCGACCG |
| | | AGGACUGACCACUCGACCAGGUUCUGGGUUGACAA |
| | | AUAUCAAGACGGAGGAGAUCUCUGAAGUGAAGAUG |
| | | GAUGCAGAAUUCCGACAUGACUCAGGAUAUGAAGU |
| | | UCAUCAUCAAAAAUUGGUGUUCUUUGCAGAAGAUG |
| | | UGGGUUCAAACAAAGGUGCAAUCAUUGGACUCAUG |
| | | GUGGGCGGUGUUGUCAUAGCGACAGUGAUCGUCAU |
| | | CACCUUGGUGAUGCUGAAGAAGAAACAGUACACAU |
| | | CCAUUCAUCAUGGUGUGGUGGAGGUUGACGCCGCU |
| | | GUCACCCCAGAGGAGCGCCACCUGUCCAAGAUGCA |
| | | GCAGAACGGCUACGAAAAUCCAACCUACAAGUUCU |
| | | UUGAGCAGAUGCAGAACUAGACCCCCGCCACAGCA |
| | | GCCUCUGAAGUUGGACAGCAAAACCAUUGCUUCAC |
| | | UACCCAUCGGUGUCCAUUUAUAGAAUAAUGUGGGA |
| | | AGAAACAAACCCGUUUUAUGAUUUACUCAUUAUCG |
| | | CCUUUUGACAGCUGUGCUGUAACACAAGUAGAUGC |
| | | CUGAACUUGAAUUAAUCCACACAUCAGUAAUGUAU |
| | | UCUAUCUCUCUUUACAUUUUGGUCUCUAUACUACA |
| | | UUAUUAAUGGGUUUUGUGUACUGUAAAGAAUUUAG |
| | | CUGUAUCAAACUAGUGCAUGAAUAGAUUCUCUCCU |
| | | GAUUAUUUAUCACAUAGCCCCUUAGCCAGUUGUAU |
| | | AUUAUUCUUGUGGUUUGUGACCCAAUUAAGUCCUA |
| | | CUUUACAUAUGCUUUAAGAAUCGAUGGGGGAUGCU |
| | | UCAUGUGAACGUGGGAGUUCAGCUGCUUCUCUUGC |
| | | CUAAGUAUUCCUUUCCUGAUCACUAUGCAUUUUAA |
| | | AGUUAAACAUUUUUAAGUAUUUCAGAUGCUUUAGA |
| | | GAGAUUUUUUUUCCAUGACUGCAUUUUACUGUACA |
| | | GAUUGCUGCUUCUGCUAUAUUUGUGAUAUAGGAAU |
| | | UAAGAGGAUACACACGUUUGUUUCUUCGUGCCUGU |
| | | UUUAUGUGCACACAUUAGGCAUUGAGACUUCAAGC |
| | | UUUUCUUUUUUUGUCCACGUAUCUUUGGGUCUUUG |
| | | AUAAAGAAAAGAAUCCCUGUUCAUUGUAAGCACUU |
| | | UUACGGGGCGGGUGGGGAGGGGUGCUCUGCUGGUC |
| | | UUCAAUUACCAAGAAUUCUCCAAAACAAUUUUCUG |
| | | CAGGAUGAUUGUACAGAAUCAUUGCUUAUGACAUG |
| | | AUCGCUUUCUACACUGUAUUACAUAAAUAAAUUAA |
| | | AUAAAAUAACCCCGGGCAAGACUUUUCUUUGAAGG |
| | | AUGACUACAGACAUUAAAUAAUCGAAGUAAUUUUG |
| | | GGUGGGGAGAAGAGGCAGAUUCAAUUUUCUUUAAC |
| | | CAGUCUGAAGUUUCAUUUAUGAUACAAAAGAAGAU |
| | | GAAAAUGGAAGUGGCAAUAUAAGGGGAUGAGGAAG |
| | | GCAUGCCUGGACAAACCCUUCUUUUAAGAUGUGUC |
| | | UUCAAUUUGUAUAAAAUGGUGUUUUCAUGUAAAUA |
| | | AAUACAUUCUUGGAGGAGCA |
| 9 | 7 | GUCGGAUGAUUCAAGCUCACGGGGACGAGCAGGAG |
| | | CGCUCUCGACUUUUCUAGAGCCUCAGCGUCCUAGG |
| | | ACUCACCUUUCCCUGAUCCUGCACCGUCCCUCUCC |
| | | UGGCCCCAGACUCUCCCUCCCACUGUUCACGAAGC |
| | | CCAGGUACCCACUGAUGGUAAUGCUGGCCUGCUGG |
| | | CUGAACCCCAGAUUGCCAUGUUCUGUGGCAGACUG |
| | | AACAUGCACAUGAAUGUCCAGAAUGGGAAGUGGGA |
| | | UUCAGAUCCAUCAGGGACCAAAACCUGCAUUGAUA |
| | | CCAAGGAAGGCAUCCUGCAGUAUUGCCAAGAAGUC |
| | | UACCCUGAACUGCAGAUCACCAAUGUGGUAGAAGC |
| | | CAACCAACCAGUGACCAUCCAGAACUGGUGCAAGC |
| | | GGGGCCGCAAGCAGUGCAAGACCCAUCCCCACUUU |
| | | GUGAUUCCCUACCGCUGCUUAGUUGGUGAGUUUGU |
| | | AAGUGAUGCCCUUCUCGUUCCUGACAAGUGCAAAU |
| | | UCUUACACCAGGAGAGGAUGGAUGUUUGCGAAACU |
| | | CAUCUUCACUGGCACACCGUCGCCAAAGAGACAUG |
| | | CAGUGAGAAGAGUACCAACUUGCAUGACUACGGCA |
| | | UGUUGCUGCCCUGCGGAAUUGACAAGUUCCGAGGG |
| | | GUAGAGUUUGUGUGUUGCCCACUGGCUGAAGAAAG |
| | | UGACAAUGUGGAUUCUGCUGAUGCGGAGGAGGAUG |
| | | ACUCGGAUGUCUGGUGGGGCGGAGCAGACACAGAC |
| | | UAUGCAGAUGGGAGUGAAGACAAAGUAGUAGAAGU |
| | | AGCAGAGGAGGAAGAAGUGGCUGAGGUGGAAGAAG |
| | | AAGAAGCCGAUGAUGACGAGGACGAUGAGGAUGGU |
| | | GAUGAGGUAGAGGAAGAGGCUGAGGAACCCUACGA |
| | | AGAAGCCACAGAGAGAACCACCAGCAUUGCCACCA |
| | | CCACCACCACCACCACAGAGUCUGUGGAAGAGGUG |
| | | GUUCGAGUUCCUACAACAGCAGCCAGUACCCCUGA |
| | | UGCCGUUGACAAGUAUCUCGAGACACCUGGGGAUG |
| | | AGAAUGAACAUGCCCAUUUCCAGAAAGCCAAAGAG |
| | | AGGCUUGAGGCCAAGCACCGAGAGAGAAUGUCCCA |
| | | GGUCAUGAGAGAAUGGGAAGAGGCAGAACGUCAAG |
| | | CAAAGAACUUGCCUAAAGCUGAUAAGAAGGCAGUU |
| | | AUCCAGCAUUUCCAGGAGAAAGUGGAAUCUUUGGA |
| | | ACAGGAAGCAGCCAACGAGAGACAGCAGCUGGUGG |
| | | AGACACACAUGGCCAGAGUGGAAGCCAUGCUCAAU |
| | | GACCGCCGCCGCCUGGCCCUGGAGAACUACAUCAC |
| | | CGCUCUGCAGGCUGUUCCUCCUCGGCCUCGUCACG |
| | | UGUUCAAUAUGCUAAAGAAGUAUGUCCGCGCAGAA |
| | | CAGAAGGACAGACAGCACACCCUAAAGCAUUUCGA |
| | | GCAUGUGCGCAUGGUGGAUCCCAAGAAAGCCGCUC |
| | | AGAUCCGGUCCCAGGUUAUGACACACCUCCGUGUG |
| | | AUUUAUGAGCGCAUGAAUCAGUCUCUCUCCCUGCU |
| | | CUACAACGUGCCUGCAGUGGCCGAGGAGAUUCAGG |
| | | AUGAAGUUGAUGAGCUGCUUCAGAAAGAGCAAAAC |
| | | UAUUCAGAUGACGUCUUGGCCAACAUGAUUAGUGA |
| | | ACCAAGGAUCAGUUACGGAAACGAUGCUCUCAUGC |
| | | CAUCUUUGACCGAAACGAAAACCACCGUGGAGCUC |
| | | CUUCCCGUGAAUGGAGAGUUCAGCCUGGACGAUCU |
| | | CCAGCCGUGGCAUUCUUUUGGGGCUGACUCUGUGC |
| | | CAGCCAACACAGAAAACGAAGUUGAGCCUGUUGAU |
| | | GCCCGCCCUGCUGCCGACCGAGGACUGACCACUCG |
| | | ACCAGGUUCUGGGUUGACAAAUAUCAAGACGGAGG |
| | | AGAUCUCUGAAGUGAAGAUGGAUGCAGAAUUCCGA |
| | | CAUGACUCAGGAUAUGAAGUUCAUCAUCAAAAAUU |
| | | GGUGUUCUUUGCAGAAGAUGUGGGUUCAAACAAAG |
| | | GUGCAAUCAUUGGACUCAUGGUGGGCGGUGUUGUC |
| | | AUAGCGACAGUGAUCGUCAUCACCUUGGUGAUGCU |
| | | GAAGAAGAAACAGUACACAUCCAUUCAUCAUGGUG |
| | | UGGUGGAGGUUGACGCCGCUGUCACCCCAGAGGAG |
| | | CGCCACCUGUCCAAGAUGCAGCAGAACGGCUACGA |
| | | AAAUCCAACCUACAAGUUCUUUGAGCAGAUGCAGA |
| | | ACUAGACCCCCGCCACAGCAGCCUCUGAAGUUGGA |
| | | CAGCAAAACCAUUGCUUCACUACCCAUCGGUGUCC |
| | | AUUUAUAGAAUAAUGUGGGAAGAAACAAACCCGUU |
| | | UUAUGAUUUACUCAUUAUCGCCUUUUGACAGCUGU |
| | | GCUGUAACACAAGUAGAUGCCUGAACUUGAAUUAA |
| | | UCCACACAUCAGUAAUGUAUUCUAUCUCUCUUUAC |
| | | AUUUUGGUCUCUAUACUACAUUAUUAAUGGGUUUU |
| | | GUGUACUGUAAAGAAUUUAGCUGUAUCAAACUAGU |
| | | GCAUGAAUAGAUUCUCUCCUGAUUAUUUAUCACAU |
| | | AGCCCCUUAGCCAGUUGUAUAUUAUUCUUGUGGUU |
| | | UGUGACCCAAUUAAGUCCUACUUUACAUAUGCUUU |
| | | AAGAAUCGAUGGGGGAUGCUUCAUGUGAACGUGGG |
| | | AGUUCAGCUGCUUCUCUUGCCUAAGUAUUCCUUUC |
| | | CUGAUCACUAUGCAUUUUAAAGUUAAACAUUUUUA |
| | | AGUAUUUCAGAUGCUUUAGAGAGAUUUUUUUUCCA |
| | | UGACUGCAUUUUACUGUACAGAUUGCUGCUUCUGC |
| | | UAUAUUUGUGAUAUAGGAAUUAAGAGGAUACACAC |
| | | GUUUGUUUCUUCGUGCCUGUUUUAUGUGCACACAU |
| | | UAGGCAUUGAGACUUCAAGCUUUUCUUUUUUUGUC |
| | | CACGUAUCUUUGGGUCUUUGAUAAAGAAAAGAAUC |
| | | CCUGUUCAUUGUAAGCACUUUUACGGGGCGGGUGG |
| | | GGAGGGGUGCUCUGCUGGUCUUCAAUUACCAAGAA |
| | | UUCUCCAAAACAAUUUUCUGCAGGAUGAUUGUACA |
| | | GAAUCAUUGCUUAUGACAUGAUCGCUUUCUACACU |
| | | GUAUUACAUAAAUAAAUUAAAUAAAAUAACCCCGG |
| | | GCAAGACUUUUCUUUGAAGGAUGACUACAGACAUU |
| | | AAAUAAUCGAAGUAAUUUUGGGUGGGGAGAAGAGG |
| | | CAGAUUCAAUUUUCUUUAACCAGUCUGAAGUUUCA |
| | | UUUAUGAUACAAAAGAAGAUGAAAAUGGAAGUGGC |
| | | AAUAUAAGGGGAUGAGGAAGGCAUGCCUGGACAAA |
| | | CCCUUCUUUUAAGAUGUGUCUUCAAUUUGUAUAAA |
| | | AUGGUGUUUUCAUGUAAAUAAAUACAUUCUUGGAG |
| | | GAGCA |
| 10 | 8 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | AGGUGUGCUCUGAACAAGCCGAGACGGGGCCGUGC |
| | | CGAGCAAUGAUCUCCCGCUGGUACUUUGAUGUGAC |
| | | UGAAGGGAAGUGUGCCCCAUUCUUUUACGGCGGAU |
| | | GUGGCGGCAACCGGAACAACUUUGACACAGAAGAG |
| | | UACUGCAUGGCCGUGUGUGGCAGCGCCAUGUCCCA |
| | | AAGUUUACUCAAGACUACCCAGGAACCUCUUGCCC |
| | | GAGAUCCUGUUAAACUUCCUACAACAGCAGCCAGU |
| | | ACCCCUGAUGCCGUUGACAAGUAUCUCGAGACACC |
| | | UGGGGAUGAGAAUGAACAUGCCCAUUUCCAGAAAG |
| | | CCAAAGAGAGGCUUGAGGCCAAGCACCGAGAGAGA |
| | | AUGUCCCAGGUCAUGAGAGAAUGGGAAGAGGCAGA |
| | | ACGUCAAGCAAAGAACUUGCCUAAAGCUGAUAAGA |
| | | AGGCAGUUAUCCAGCAUUUCCAGGAGAAAGUGGAA |
| | | UCUUUGGAACAGGAAGCAGCCAACGAGAGACAGCA |
| | | GCUGGUGGAGACACACAUGGCCAGAGUGGAAGCCA |
| | | UGCUCAAUGACCGCCGCCGCCUGGCCCUGGAGAAC |
| | | UACAUCACCGCUCUGCAGGCUGUUCCUCCUCGGCC |
| | | UCGUCACGUGUUCAAUAUGCUAAAGAAGUAUGUCC |
| | | GCGCAGAACAGAAGGACAGACAGCACACCCUAAAG |
| | | CAUUUCGAGCAUGUGCGCAUGGUGGAUCCCAAGAA |
| | | AGCCGCUCAGAUCCGGUCCCAGGUUAUGACACACC |
| | | UCCGUGUGAUUUAUGAGCGCAUGAAUCAGUCUCUC |
| | | UCCCUGCUCUACAACGUGCCUGCAGUGGCCGAGGA |
| | | GAUUCAGGAUGAAGUUGAUGAGCUGCUUCAGAAAG |
| | | AGCAAAACUAUUCAGAUGACGUCUUGGCCAACAUG |
| | | AUUAGUGAACCAAGGAUCAGUUACGGAAACGAUGC |
| | | UCUCAUGCCAUCUUUGACCGAAACGAAAACCACCG |
| | | UGGAGCUCCUUCCCGUGAAUGGAGAGUUCAGCCUG |
| | | GACGAUCUCCAGCCGUGGCAUUCUUUUGGGGCUGA |
| | | CUCUGUGCCAGCCAACACAGAAAACGAAGGUUCUG |
| | | GGUUGACAAAUAUCAAGACGGAGGAGAUCUCUGAA |
| | | GUGAAGAUGGAUGCAGAAUUCCGACAUGACUCAGG |
| | | AUAUGAAGUUCAUCAUCAAAAAUUGGUGUUCUUUG |
| | | CAGAAGAUGUGGGUUCAAACAAAGGUGCAAUCAUU |
| | | GGACUCAUGGUGGGCGGUGUUGUCAUAGCGACAGU |
| | | GAUCGUCAUCACCUUGGUGAUGCUGAAGAAGAAAC |
| | | AGUACACAUCCAUUCAUCAUGGUGUGGUGGAGGUU |
| | | GACGCCGCUGUCACCCCAGAGGAGCGCCACCUGUC |
| | | CAAGAUGCAGCAGAACGGCUACGAAAAUCCAACCU |
| | | ACAAGUUCUUUGAGCAGAUGCAGAACUAGACCCCC |
| | | GCCACAGCAGCCUCUGAAGUUGGACAGCAAAACCA |
| | | UUGCUUCACUACCCAUCGGUGUCCAUUUAUAGAAU |
| | | AAUGUGGGAAGAAACAAACCCGUUUUAUGAUUUAC |
| | | UCAUUAUCGCCUUUUGACAGCUGUGCUGUAACACA |
| | | AGUAGAUGCCUGAACUUGAAUUAAUCCACACAUCA |
| | | GUAAUGUAUUCUAUCUCUCUUUACAUUUUGGUCUC |
| | | UAUACUACAUUAUUAAUGGGUUUUGUGUACUGUAA |
| | | AGAAUUUAGCUGUAUCAAACUAGUGCAUGAAUAGA |
| | | UUCUCUCCUGAUUAUUUAUCACAUAGCCCCUUAGC |
| | | CAGUUGUAUAUUAUUCUUGUGGUUUGUGACCCAAU |
| | | UAAGUCCUACUUUACAUAUGCUUUAAGAAUCGAUG |
| | | GGGGAUGCUUCAUGUGAACGUGGGAGUUCAGCUGC |
| | | UUCUCUUGCCUAAGUAUUCCUUUCCUGAUCACUAU |
| | | GCAUUUUAAAGUUAAACAUUUUUAAGUAUUUCAGA |
| | | UGCUUUAGAGAGAUUUUUUUUCCAUGACUGCAUUU |
| | | UACUGUACAGAUUGCUGCUUCUGCUAUAUUUGUGA |
| | | UAUAGGAAUUAAGAGGAUACACACGUUUGUUUCUU |
| | | CGUGCCUGUUUUAUGUGCACACAUUAGGCAUUGAG |
| | | ACUUCAAGCUUUUCUUUUUUUGUCCACGUAUCUUU |
| | | GGGUCUUUGAUAAAGAAAAGAAUCCCUGUUCAUUG |
| | | UAAGCACUUUUACGGGGCGGGUGGGGAGGGGUGCU |
| | | CUGCUGGUCUUCAAUUACCAAGAAUUCUCCAAAAC |
| | | AAUUUUCUGCAGGAUGAUUGUACAGAAUCAUUGCU |
| | | UAUGACAUGAUCGCUUUCUACACUGUAUUACAUAA |
| | | AUAAAUUAAAUAAAAUAACCCCGGGCAAGACUUUU |
| | | CUUUGAAGGAUGACUACAGACAUUAAAUAAUCGAA |
| | | GUAAUUUUGGGUGGGGAGAAGAGGCAGAUUCAAUU |
| | | UUCUUUAACCAGUCUGAAGUUUCAUUUAUGAUACA |
| | | AAAGAAGAUGAAAAUGGAAGUGGCAAUAUAAGGGG |
| | | AUGAGGAAGGCAUGCCUGGACAAACCCUUCUUUUA |
| | | AGAUGUGUCUUCAAUUUGUAUAAAAUGGUGUUUUC |
| | | AUGUAAAUAAAUACAUUCUUGGAGGAGCA |
| 11 | 9 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | AGGUGUGCUCUGAACAAGCCGAGACGGGGCCGUGC |
| | | CGAGCAAUGAUCUCCCGCUGGUACUUUGAUGUGAC |
| | | UGAAGGGAAGUGUGCCCCAUUCUUUUACGGCGGAU |
| | | GUGGCGGCAACCGGAACAACUUUGACACAGAAGAG |
| | | UACUGCAUGGCCGUGUGUGGCAGCGCCAUUCCUAC |
| | | AACAGCAGCCAGUACCCCUGAUGCCGUUGACAAGU |
| | | AUCUCGAGACACCUGGGGAUGAGAAUGAACAUGCC |
| | | CAUUUCCAGAAAGCCAAAGAGAGGCUUGAGGCCAA |
| | | GCACCGAGAGAGAAUGUCCCAGGUCAUGAGAGAAU |
| | | GGGAAGAGGCAGAACGUCAAGCAAAGAACUUGCCU |
| | | AAAGCUGAUAAGAAGGCAGUUAUCCAGCAUUUCCA |
| | | GGAGAAAGUGGAAUCUUUGGAACAGGAAGCAGCCA |
| | | ACGAGAGACAGCAGCUGGUGGAGACACACAUGGCC |
| | | AGAGUGGAAGCCAUGCUCAAUGACCGCCGCCGCCU |
| | | GGCCCUGGAGAACUACAUCACCGCUCUGCAGGCUG |
| | | UUCCUCCUCGGCCUCGUCACGUGUUCAAUAUGCUA |
| | | AAGAAGUAUGUCCGCGCAGAACAGAAGGACAGACA |
| | | GCACACCCUAAAGCAUUUCGAGCAUGUGCGCAUGG |
| | | UGGAUCCCAAGAAAGCCGCUCAGAUCCGGUCCCAG |
| | | GUUAUGACACACCUCCGUGUGAUUUAUGAGCGCAU |
| | | GAAUCAGUCUCUCUCCCUGCUCUACAACGUGCCUG |
| | | CAGUGGCCGAGGAGAUUCAGGAUGAAGUUGAUGAG |
| | | CUGCUUCAGAAAGAGCAAAACUAUUCAGAUGACGU |
| | | CUUGGCCAACAUGAUUAGUGAACCAAGGAUCAGUU |
| | | ACGGAAACGAUGCUCUCAUGCCAUCUUUGACCGAA |
| | | ACGAAAACCACCGUGGAGCUCCUUCCCGUGAAUGG |
| | | AGAGUUCAGCCUGGACGAUCUCCAGCCGUGGCAUU |
| | | CUUUUGGGGCUGACUCUGUGCCAGCCAACACAGAA |
| | | AACGAAGGUUCUGGGUUGACAAAUAUCAAGACGGA |
| | | GGAGAUCUCUGAAGUGAAGAUGGAUGCAGAAUUCC |
| | | GACAUGACUCAGGAUAUGAAGUUCAUCAUCAAAAA |
| | | UUGGUGUUCUUUGCAGAAGAUGUGGGUUCAAACAA |
| | | AGGUGCAAUCAUUGGACUCAUGGUGGGCGGUGUUG |
| | | UCAUAGCGACAGUGAUCGUCAUCACCUUGGUGAUG |
| | | CUGAAGAAGAAACAGUACACAUCCAUUCAUCAUGG |
| | | UGUGGUGGAGGUUGACGCCGCUGUCACCCCAGAGG |
| | | AGCGCCACCUGUCCAAGAUGCAGCAGAACGGCUAC |
| | | GAAAAUCCAACCUACAAGUUCUUUGAGCAGAUGCA |
| | | GAACUAGACCCCCGCCACAGCAGCCUCUGAAGUUG |
| | | GACAGCAAAACCAUUGCUUCACUACCCAUCGGUGU |
| | | CCAUUUAUAGAAUAAUGUGGGAAGAAACAAACCCG |
| | | UUUUAUGAUUUACUCAUUAUCGCCUUUUGACAGCU |
| | | GUGCUGUAACACAAGUAGAUGCCUGAACUUGAAUU |
| | | AAUCCACACAUCAGUAAUGUAUUCUAUCUCUCUUU |
| | | ACAUUUUGGUCUCUAUACUACAUUAUUAAUGGGUU |
| | | UUGUGUACUGUAAAGAAUUUAGCUGUAUCAAACUA |
| | | GUGCAUGAAUAGAUUCUCUCCUGAUUAUUUAUCAC |
| | | AUAGCCCCUUAGCCAGUUGUAUAUUAUUCUUGUGG |
| | | UUUGUGACCCAAUUAAGUCCUACUUUACAUAUGCU |
| | | UUAAGAAUCGAUGGGGGAUGCUUCAUGUGAACGUG |
| | | GGAGUUCAGCUGCUUCUCUUGCCUAAGUAUUCCUU |
| | | UCCUGAUCACUAUGCAUUUUAAAGUUAAACAUUUU |
| | | UAAGUAUUUCAGAUGCUUUAGAGAGAUUUUUUUUC |
| | | CAUGACUGCAUUUUACUGUACAGAUUGCUGCUUCU |
| | | GCUAUAUUUGUGAUAUAGGAAUUAAGAGGAUACAC |
| | | ACGUUUGUUUCUUCGUGCCUGUUUUAUGUGCACAC |
| | | AUUAGGCAUUGAGACUUCAAGCUUUUCUUUUUUUG |
| | | UCCACGUAUCUUUGGGUCUUUGAUAAAGAAAAGAA |
| | | UCCCUGUUCAUUGUAAGCACUUUUACGGGGCGGGU |
| | | GGGGAGGGGUGCUCUGCUGGUCUUCAAUUACCAAG |
| | | AAUUCUCCAAAACAAUUUUCUGCAGGAUGAUUGUA |
| | | CAGAAUCAUUGCUUAUGACAUGAUCGCUUUCUACA |
| | | CUGUAUUACAUAAAUAAAUUAAAUAAAAUAACCCC |
| | | GGGCAAGACUUUUCUUUGAAGGAUGACUACAGACA |
| | | UUAAAUAAUCGAAGUAAUUUUGGGUGGGGAGAAGA |
| | | GGCAGAUUCAAUUUUCUUUAACCAGUCUGAAGUUU |
| | | CAUUUAUGAUACAAAAGAAGAUGAAAAUGGAAGUG |
| | | GCAAUAUAAGGGGAUGAGGAAGGCAUGCCUGGACA |
| | | AACCCUUCUUUUAAGAUGUGUCUUCAAUUUGUAUA |
| | | AAAUGGUGUUUUCAUGUAAAUAAAUACAUUCUUGG |
| | | AGGAGCA |
| 12 | 10 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | UUCCUACAACAGCAGCCAGUACCCCUGAUGCCGUU |
| | | GACAAGUAUCUCGAGACACCUGGGGAUGAGAAUGA |
| | | ACAUGCCCAUUUCCAGAAAGCCAAAGAGAGGCUUG |
| | | AGGCCAAGCACCGAGAGAGAAUGUCCCAGGUCAUG |
| | | AGAGAAUGGGAAGAGGCAGAACGUCAAGCAAAGAA |
| | | CUUGCCUAAAGCUGAUAAGAAGGCAGUUAUCCAGC |
| | | AUUUCCAGGAGAAAGUGGAAUCUUUGGAACAGGAA |
| | | GCAGCCAACGAGAGACAGCAGCUGGUGGAGACACA |
| | | CAUGGCCAGAGUGGAAGCCAUGCUCAAUGACCGCC |
| | | GCCGCCUGGCCCUGGAGAACUACAUCACCGCUCUG |
| | | CAGGCUGUUCCUCCUCGGCCUCGUCACGUGUUCAA |
| | | UAUGCUAAAGAAGUAUGUCCGCGCAGAACAGAAGG |
| | | ACAGACAGCACACCCUAAAGCAUUUCGAGCAUGUG |
| | | CGCAUGGUGGAUCCCAAGAAAGCCGCUCAGAUCCG |
| | | GUCCCAGGUUAUGACACACCUCCGUGUGAUUUAUG |
| | | AGCGCAUGAAUCAGUCUCUCUCCCUGCUCUACAAC |
| | | GUGCCUGCAGUGGCCGAGGAGAUUCAGGAUGAAGU |
| | | UGAUGAGCUGCUUCAGAAAGAGCAAAACUAUUCAG |
| | | AUGACGUCUUGGCCAACAUGAUUAGUGAACCAAGG |
| | | AUCAGUUACGGAAACGAUGCUCUCAUGCCAUCUUU |
| | | GACCGAAACGAAAACCACCGUGGAGCUCCUUCCCG |
| | | UGAAUGGAGAGUUCAGCCUGGACGAUCUCCAGCCG |
| | | UGGCAUUCUUUUGGGGCUGACUCUGUGCCAGCCAA |
| | | CACAGAAAACGAAGGUUCUGGGUUGACAAAUAUCA |
| | | AGACGGAGGAGAUCUCUGAAGUGAAGAUGGAUGCA |
| | | GAAUUCCGACAUGACUCAGGAUAUGAAGUUCAUCA |
| | | UCAAAAAUUGGUGUUCUUUGCAGAAGAUGUGGGUU |
| | | CAAACAAAGGUGCAAUCAUUGGACUCAUGGUGGGC |
| | | GGUGUUGUCAUAGCGACAGUGAUCGUCAUCACCUU |
| | | GGUGAUGCUGAAGAAGAAACAGUACACAUCCAUUC |
| | | AUCAUGGUGUGGUGGAGGUUGACGCCGCUGUCACC |
| | | CCAGAGGAGCGCCACCUGUCCAAGAUGCAGCAGAA |
| | | CGGCUACGAAAAUCCAACCUACAAGUUCUUUGAGC |
| | | AGAUGCAGAACUAGACCCCCGCCACAGCAGCCUCU |
| | | GAAGUUGGACAGCAAAACCAUUGCUUCACUACCCA |
| | | UCGGUGUCCAUUUAUAGAAUAAUGUGGGAAGAAAC |
| | | AAACCCGUUUUAUGAUUUACUCAUUAUCGCCUUUU |
| | | GACAGCUGUGCUGUAACACAAGUAGAUGCCUGAAC |
| | | UUGAAUUAAUCCACACAUCAGUAAUGUAUUCUAUC |
| | | UCUCUUUACAUUUUGGUCUCUAUACUACAUUAUUA |
| | | AUGGGUUUUGUGUACUGUAAAGAAUUUAGCUGUAU |
| | | CAAACUAGUGCAUGAAUAGAUUCUCUCCUGAUUAU |
| | | UUAUCACAUAGCCCCUUAGCCAGUUGUAUAUUAUU |
| | | CUUGUGGUUUGUGACCCAAUUAAGUCCUACUUUAC |
| | | AUAUGCUUUAAGAAUCGAUGGGGGAUGCUUCAUGU |
| | | GAACGUGGGAGUUCAGCUGCUUCUCUUGCCUAAGU |
| | | AUUCCUUUCCUGAUCACUAUGCAUUUUAAAGUUAA |
| | | ACAUUUUUAAGUAUUUCAGAUGCUUUAGAGAGAUU |
| | | UUUUUUCCAUGACUGCAUUUUACUGUACAGAUUGC |
| | | UGCUUCUGCUAUAUUUGUGAUAUAGGAAUUAAGAG |
| | | GAUACACACGUUUGUUUCUUCGUGCCUGUUUUAUG |
| | | UGCACACAUUAGGCAUUGAGACUUCAAGCUUUUCU |
| | | UUUUUUGUCCACGUAUCUUUGGGUCUUUGAUAAAG |
| | | AAAAGAAUCCCUGUUCAUUGUAAGCACUUUUACGG |
| | | GGCGGGUGGGGAGGGGUGCUCUGCUGGUCUUCAAU |
| | | UACCAAGAAUUCUCCAAAACAAUUUUCUGCAGGAU |
| | | GAUUGUACAGAAUCAUUGCUUAUGACAUGAUCGCU |
| | | UUCUACACUGUAUUACAUAAAUAAAUUAAAUAAAA |
| | | UAACCCCGGGCAAGACUUUUCUUUGAAGGAUGACU |
| | | ACAGACAUUAAAUAAUCGAAGUAAUUUUGGGUGGG |
| | | GAGAAGAGGCAGAUUCAAUUUUCUUUAACCAGUCU |
| | | GAAGUUUCAUUUAUGAUACAAAAGAAGAUGAAAAU |
| | | GGAAGUGGCAAUAUAAGGGGAUGAGGAAGGCAUGC |
| | | CUGGACAAACCCUUCUUUUAAGAUGUGUCUUCAAU |
| | | UUGUAUAAAAUGGUGUUUUCAUGUAAAUAAAUACA |
| | | UUCUUGGAGGAGCA |
| 13 | 11 | GUCAGUUUCCUCGGCAGCGGUAGGCGAGAGCACGC |
| | | GGAGGAGCGUGCGCGGGGGCCCCGGGAGACGGCGG |
| | | CGGUGGCGGCGCGGGCAGAGCAAGGACGCGGCGGA |
| | | UCCCACUCGCACAGCAGCGCACUCGGUGCCCCGCG |
| | | CAGGGUCGCGAUGCUGCCCGGUUUGGCACUGCUCC |
| | | UGCUGGCCGCCUGGACGGCUCGGGCGCUGGAGGUA |
| | | CCCACUGAUGGUAAUGCUGGCCUGCUGGCUGAACC |
| | | CCAGAUUGCCAUGUUCUGUGGCAGACUGAACAUGC |
| | | ACAUGAAUGUCCAGAAUGGGAAGUGGGAUUCAGAU |
| | | CCAUCAGGGACCAAAACCUGCAUUGAUACCAAGGA |
| | | AGGCAUCCUGCAGUAUUGCCAAGAAGUCUACCCUG |
| | | AACUGCAGAUCACCAAUGUGGUAGAAGCCAACCAA |
| | | CCAGUGACCAUCCAGAACUGGUGCAAGCGGGGCCG |
| | | CAAGCAGUGCAAGACCCAUCCCCACUUUGUGAUUC |
| | | CCUACCGCUGCUUAGUUGGUGAGUUUGUAAGUGAU |
| | | GCCCUUCUCGUUCCUGACAAGUGCAAAUUCUUACA |
| | | CCAGGAGAGGAUGGAUGUUUGCGAAACUCAUCUUC |
| | | ACUGGCACACCGUCGCCAAAGAGACAUGCAGUGAG |
| | | AAGAGUACCAACUUGCAUGACUACGGCAUGUUGCU |
| | | GCCCUGCGGAAUUGACAAGUUCCGAGGGGUAGAGU |
| | | UUGUGUGUUGCCCACUGGCUGAAGAAAGUGACAAU |
| | | GUGGAUUCUGCUGAUGCGGAGGAGGAUGACUCGGA |
| | | UGUCUGGUGGGGCGGAGCAGACACAGACUAUGCAG |
| | | AUGGGAGUGAAGACAAAGUAGUAGAAGUAGCAGAG |
| | | GAGGAAGAAGUGGCUGAGGUGGAAGAAGAAGAAGC |
| | | CGAUGAUGACGAGGACGAUGAGGAUGGUGAUGAGG |
| | | UAGAGGAAGAGGCUGAGGAACCCUACGAAGAAGCC |
| | | ACAGAGAGAACCACCAGCAUUGCCACCACCACCAC |
| | | CACCACCACAGAGUCUGUGGAAGAGGUGGUUCGAG |
| | | UGUCCCAAAGUUUACUCAAGACUACCCAGGAACCU |
| | | CUUGCCCGAGAUCCUGUUAAACUUCCUACAACAGC |
| | | AGCCAGUACCCCUGAUGCCGUUGACAAGUAUCUCG |
| | | AGACACCUGGGGAUGAGAAUGAACAUGCCCAUUUC |
| | | CAGAAAGCCAAAGAGAGGCUUGAGGCCAAGCACCG |
| | | AGAGAGAAUGUCCCAGGUCAUGAGAGAAUGGGAAG |
| | | AGGCAGAACGUCAAGCAAAGAACUUGCCUAAAGCU |
| | | GAUAAGAAGGCAGUUAUCCAGCAUUUCCAGGAGAA |
| | | AGUGGAAUCUUUGGAACAGGAAGCAGCCAACGAGA |
| | | GACAGCAGCUGGUGGAGACACACAUGGCCAGAGUG |
| | | GAAGCCAUGCUCAAUGACCGCCGCCGCCUGGCCCU |
| | | GGAGAACUACAUCACCGCUCUGCAGGCUGUUCCUC |
| | | CUCGGCCUCGUCACGUGUUCAAUAUGCUAAAGAAG |
| | | UAUGUCCGCGCAGAACAGAAGGACAGACAGCACAC |
| | | CCUAAAGCAUUUCGAGCAUGUGCGCAUGGUGGAUC |
| | | CCAAGAAAGCCGCUCAGAUCCGGUCCCAGGUUAUG |
| | | ACACACCUCCGUGUGAUUUAUGAGCGCAUGAAUCA |
| | | GUCUCUCUCCCUGCUCUACAACGUGCCUGCAGUGG |
| | | CCGAGGAGAUUCAGGAUGAAGUUGAUGAGCUGCUU |
| | | CAGAAAGAGCAAAACUAUUCAGAUGACGUCUUGGC |
| | | CAACAUGAUUAGUGAACCAAGGAUCAGUUACGGAA |
| | | ACGAUGCUCUCAUGCCAUCUUUGACCGAAACGAAA |
| | | ACCACCGUGGAGCUCCUUCCCGUGAAUGGAGAGUU |
| | | CAGCCUGGACGAUCUCCAGCCGUGGCAUUCUUUUG |
| | | GGGCUGACUCUGUGCCAGCCAACACAGAAAACGAA |
| | | GUUGAGCCUGUUGAUGCCCGCCCUGCUGCCGACCG |
| | | AGGACUGACCACUCGACCAGGUUCUGGGUUGACAA |
| | | AUAUCAAGACGGAGGAGAUCUCUGAAGUGAAGAUG |
| | | GAUGCAGAAUUCCGACAUGACUCAGGAUAUGAAGU |
| | | UCAUCAUCAAAAAUUGGUGUUCUUUGCAGAAGAUG |
| | | UGGGUUCAAACAAAGGUGCAAUCAUUGGACUCAUG |
| | | GUGGGCGGUGUUGUCAUAGCGACAGUGAUCGUCAU |
| | | CACCUUGGUGAUGCUGAAGAAGAAACAGUACACAU |
| | | CCAUUCAUCAUGGUGUGGUGGAGGUUGACGCCGCU |
| | | GUCACCCCAGAGGAGCGCCACCUGUCCAAGAUGCA |
| | | GCAGAACGGCUACGAAAAUCCAACCUACAAGUUCU |
| | | UUGAGCAGAUGCAGAACUAGACCCCCGCCACAGCA |
| | | GCCUCUGAAGUUGGACAGCAAAACCAUUGCUUCAC |
| | | UACCCAUCGGUGUCCAUUUAUAGAAUAAUGUGGGA |
| | | AGAAACAAACCCGUUUUAUGAUUUACUCAUUAUCG |
| | | CCUUUUGACAGCUGUGCUGUAACACAAGUAGAUGC |
| | | CUGAACUUGAAUUAAUCCACACAUCAGUAAUGUAU |
| | | UCUAUCUCUCUUUACAUUUUGGUCUCUAUACUACA |
| | | UUAUUAAUGGGUUUUGUGUACUGUAAAGAAUUUAG |
| | | CUGUAUCAAACUAGUGCAUGAAUAGAUUCUCUCCU |
| | | GAUUAUUUAUCACAUAGCCCCUUAGCCAGUUGUAU |
| | | AUUAUUCUUGUGGUUUGUGACCCAAUUAAGUCCUA |
| | | CUUUACAUAUGCUUUAAGAAUCGAUGGGGGAUGCU |
| | | UCAUGUGAACGUGGGAGUUCAGCUGCUUCUCUUGC |
| | | CUAAGUAUUCCUUUCCUGAUCACUAUGCAUUUUAA |
| | | AGUUAAACAUUUUUAAGUAUUUCAGAUGCUUUAGA |
| | | GAGAUUUUUUUUCCAUGACUGCAUUUUACUGUACA |
| | | GAUUGCUGCUUCUGCUAUAUUUGUGAUAUAGGAAU |
| | | UAAGAGGAUACACACGUUUGUUUCUUCGUGCCUGU |
| | | UUUAUGUGCACACAUUAGGCAUUGAGACUUCAAGC |
| | | UUUUCUUUUUUUGUCCACGUAUCUUUGGGUCUUUG |
| | | AUAAAGAAAAGAAUCCCUGUUCAUUGUAAGCACUU |
| | | UUACGGGGCGGGUGGGGAGGGGUGCUCUGCUGGUC |
| | | UUCAAUUACCAAGAAUUCUCCAAAACAAUUUUCUG |
| | | CAGGAUGAUUGUACAGAAUCAUUGCUUAUGACAUG |
| | | AUCGCUUUCUACACUGUAUUACAUAAAUAAAUUAA |
| | | AUAAAAUAACCCCGGGCAAGACUUUUCUUUGAAGG |
| | | AUGACUACAGACAUUAAAUAAUCGAAGUAAUUUUG |
| | | GGUGGGGAGAAGAGGCAGAUUCAAUUUUCUUUAAC |
| | | CAGUCUGAAGUUUCAUUUAUGAUACAAAAGAAGAU |
| | | GAAAAUGGAAGUGGCAAUAUAAGGGGAUGAGGAAG |
| | | GCAUGCCUGGACAAACCCUUCUUUUAAGAUGUGUC |
| | | UUCAAUUUGUAUAAAAUGGUGUUUUCAUGUAAAUA |
| | | AAUACAUUCUUGGAGGAGCA |

An Abeta fragment can be formed by cleaving a portion of an APP. An enzyme can cleave the APP. The enzyme can be a gamma secretase, a beta secretase (e.g., BACE1, cathepsin B or Meprin beta), or a combination thereof. An Abeta fragment can be from about 30 to about 50 amino acids in length. An Abeta fragment can be from about 35 to about 45 amino acids in length. An Abeta fragment can be from about 38 to about 42 amino acids in length. An Abeta fragment can be from about 36 to about 42 amino acids in length. An Abeta fragment can be from about 40 to about 45 amino acids in length. An Abeta fragment can be from about 33 to about 40 amino acids in length. mRNA base editing on an APP can prevent cleavage of an Abeta fragment or substantially reduce cleavage of an Abeta fragment. An Abeta fragment can comprise at least about: 80%, 85%, 90%, 95%, 96%, 97%, 98%, or 99% sequence homology to SEQ ID NO: 14. An Abeta fragment can comprise at least about 80%, 85%, 90%, 95%, 96%, 97%, 98%, or 99% sequence homology to SEQ ID NO: 15.

Abeta 40-
SEQ ID NO: 14:
DAEFRHDSGYEVHHQKLVFFAEDVGSNKGAIIGLMVGGVV
Abeta 42-
SEQ ID NO. 15:
DAEFRHDSGYEVHHQKLVFFAEDVGSNKGAIIGLMVGGVVIA

A polynucleotide at least partially encoding an amyloid precursor protein is modified by the compositions disclosed herein (e.g., an engineered guide RNA targeting a cleavage site in APP) to produce a modified polynucleotide at least partially encoding a modified amyloid precursor protein. In some cases, the modified amyloid precursor protein has an altered susceptibility to protease cleavage relative to the amyloid precursor protein encoded by the unedited polynucleotide. In some cases, the unedited polynucleotide encodes at least a portion of the wild type sequence of amyloid precursor protein. In some cases, the unedited polynucleotide encodes at least a portion of the amyloid precursor protein sequence of SEQ ID NO: 2. In some embodiments, Abeta fragment can also comprise Abeta 1, Abeta 2, Abeta 3, Abeta 4, Abeta 5, Abeta 6, Abeta 7, Abeta 8, Abeta 9, Abeta 10, Abeta 11, Abeta 12, Abeta 13, Abeta 14, Abeta 15, Abeta 16, Abeta 17, Abeta 18, Abeta 19, Abeta 20, Abeta 21, Abeta 22, Abeta 23, Abeta 24, Abeta 25, Abeta 26, Abeta 27, Abeta 28, Abeta 29, Abeta 30, Abeta 31, Abeta 32, Abeta 33, Abeta 34, Abeta 35, Abeta 36, Abeta 37, Abeta 38, Abeta 39, Abeta 40, Abeta 41, Abeta 42, any derivatives herein or thereof, or any combinations herein and thereof. Aggregations of Abeta fragments can create Abeta or amyloid-beta plaques. Abeta or amyloid-beta plaques can comprise Abeta 1, Abeta 2, Abeta 3, Abeta 4, Abeta 5, Abeta 6, Abeta 7, Abeta 8, Abeta 9, Abeta 10, Abeta 11, Abeta 12, Abeta 13, Abeta 14, Abeta 15, Abeta 16, Abeta 17, Abeta 18, Abeta 19, Abeta 20, Abeta 21, Abeta 22, Abeta 23, Abeta 24, Abeta 25, Abeta 26, Abeta 27, Abeta 28, Abeta 29, Abeta 30, Abeta 31, Abeta 32, Abeta 33, Abeta 34, Abeta 35, Abeta 36, Abeta 37, Abeta 38, Abeta 39, Abeta 40, Abeta 41, Abeta 42, any derivatives herein or thereof, or any combinations herein and thereof. In some embodiments, an Abeta or amyloid-beta plaque can comprise one 1, 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, 1×10\hat{ }12, 1×10\hat{ }13, 1×10\hat{ }14, 1×10\hat{ }15, 1×10\hat{ }16, 1×10\hat{ }17, 1×10\hat{ }18, 1×10\hat{ }19, 1×10\hat{ }20, 1×10\hat{ }21, 1×10\hat{ }22, 1×10\hat{ }23, 1×10\hat{ }24, 1×10\hat{ }25, 1×10\hat{ }26, 1×10\hat{ }27, 1×10\hat{ }28, 1×10\hat{ }29, 1×10\hat{ }30, 1×10\hat{ }31, 1×10\hat{ }32, 1×10\hat{ }33, 1×10\hat{ }34, 1×10\hat{ }35, 1×10\hat{ }36, 1×10\hat{ }37, 1×10\hat{ }38, 1×10\hat{ }39, 1×10\hat{ }40, 1×10\hat{ }41, 1×10\hat{ }42, 1×10\hat{ }43, 1×10\hat{ }44, 1×10\hat{ }45, 1×10\hat{ }46, 1×10\hat{ }47, 1×10\hat{ }48, 1×10\hat{ }49, 1×10\hat{ }50, 1×10\hat{ }51, 1×10\hat{ }52, 1×10\hat{ }53, 1×10\hat{ }54, 1×10\hat{ }55, 1×10\hat{ }56, 1×10\hat{ }57, 1×10\hat{ }58, 1×10\hat{ }59, 1×10\hat{ }60, 1×10\hat{ }61, 1×10\hat{ }62, 1×10\hat{ }63, 1×10\hat{ }64, 1×10\hat{ }65, 1×10\hat{ }66, 1×10\hat{ }67, 1×10\hat{ }68, 1×10\hat{ }69, 1×10\hat{ }70, 1×10\hat{ }71, 1×10\hat{ }72, 1×10\hat{ }73, 1×10\hat{ }74, 1×10\hat{ }75, 1×10\hat{ }76, 1×10\hat{ }77, 1×10\hat{ }78, 1×10\hat{ }79, 1×10\hat{ }80, 1×10\hat{ }81, 1×10\hat{ }82, 1×10\hat{ }83, 1×10\hat{ }84, 1×10\hat{ }85, 1×10\hat{ }86, 1×10\hat{ }87, 1×10\hat{ }88, 1×10\hat{ }89, 1×10\hat{ }90, 1×10\hat{ }91, 1×10\hat{ }92, 1×10\hat{ }93, 1×10\hat{ }94, 1×10\hat{ }95, 1×10\hat{ }96, 1×10\hat{ }97, 1×10\hat{ }98, 1×10\hat{ }99, or 1×10\hat{ }100 Abeta fragments or molecules. In other cases, an Abeta or amyloid-beta plaque can comprise from 1 to 1×10\hat{ }1, from 9 to 1×10\hat{ }2, from 0.99×10\hat{ }2 to 1×10\hat{ }3, from 0.99×10\hat{ }3 to 1×10\hat{ }4, from 0.99×10\hat{ }4 to 1×10\hat{ }5, from 0.99×10\hat{ }5 to 1×10\hat{ }6, from 0.99×10\hat{ }6 to 1×10\hat{ }7, from 0.99×10\hat{ }7 to 1×10\hat{ }8, from 0.99×10\hat{ }8 to 1×10\hat{ }9, from 0.99×10\hat{ }9 to 1×10\hat{ }10, from 0.99×10\hat{ }10 to 1×10\hat{ }11, from 0.99×10\hat{ }11 to 1×10\hat{ }12, from 0.99×10\hat{ }12 to 1×10\hat{ }13, from 0.99×10\hat{ }13 to 1×10\hat{ }14, from 0.99×10\hat{ }14 to 1×10\hat{ }15, from 0.99×10\hat{ }15 to 1×10\hat{ }16, from 0.99×10\hat{ }16 to 1×10\hat{ }17, from 0.99×10\hat{ }17 to 1×10\hat{ }18, from 0.99×10\hat{ }18 to 1×10\hat{ }19, from 0.99×10\hat{ }19 to 1×10\hat{ }20, from 0.99×10\hat{ }20 to 1×10\hat{ }21, from 0.99×10\hat{ }21 to 1×10\hat{ }22, from 0.99×10\hat{ }22 to 1×10\hat{ }23, from 0.99×10\hat{ }23 to 1×10\hat{ }24, from 0.99×10\hat{ }24 to 1×10\hat{ }25, from 0.99×10\hat{ }25 to 1×10\hat{ }26, from 0.99×10\hat{ }26 to 1×10\hat{ }27, from 0.99×10\hat{ }27 to 1×10\hat{ }28, from 0.99×10\hat{ }28 to 1×10\hat{ }29, from 0.99×10\hat{ }29 to 1×10\hat{ }30, from 0.99×10\hat{ }30 to 1×10\hat{ }31, from 0.99×10\hat{ }31 to 1×10\hat{ }32, from 0.99×10\hat{ }32 to 1×10\hat{ }33, from 0.99×10\hat{ }33 to 1×10\hat{ }34, from 0.99×10\hat{ }34 to 1×10\hat{ }35, from 0.99×10\hat{ }35 to 1×10\hat{ }36, from 0.99×10\hat{ }36 to 1×10\hat{ }37, from 0.99×10\hat{ }37 to 1×10\hat{ }38, from 0.99×10\hat{ }38 to 1×10\hat{ }39, from 0.99×10\hat{ }39 to 1×10\hat{ }40, from 0.99×10\hat{ }40 to 1×10\hat{ }41, from 0.99×10\hat{ }41 to 1×10\hat{ }42, from 0.99×10\hat{ }42 to 1×10\hat{ }43, from 0.99×10\hat{ }43 to 1×10\hat{ }44, from 0.99×10\hat{ }44 to 1×10\hat{ }45, from 0.99×10\hat{ }45 to 1×10\hat{ }46, from 0.99×10\hat{ }46 to 1×10\hat{ }47, from 0.99×10\hat{ }47 to 1×10\hat{ }48, from 0.99×10\hat{ }48 to 1×10\hat{ }49, from 0.99×10\hat{ }49 to 1×10\hat{ }50, from 0.99×10\hat{ }50 to 1×10\hat{ }51, from 0.99×10\hat{ }51 to 1×10\hat{ }52, from 0.99×10\hat{ }52 to 1×10\hat{ }53, from 0.99×10\hat{ }53 to 1×10\hat{ }54, from 0.99×10\hat{ }54 to 1×10\hat{ }55, from 0.99×10\hat{ }55 to 1×10\hat{ }56, from 0.99×10\hat{ }56 to 1×10\hat{ }57, from 0.99×10\hat{ }57 to 1×10\hat{ }58, from 0.99×10\hat{ }58 to 1×10\hat{ }59, from 0.99×10\hat{ }59 to 1×10\hat{ }60, from 0.99×10\hat{ }60 to 1×10\hat{ }61, from 0.99×10\hat{ }61 to 1×10\hat{ }62, from 0.99×10\hat{ }62 to 1×10\hat{ }63, from 0.99×10\hat{ }63 to 1×10\hat{ }64, from 0.99×10\hat{ }64 to 1×10\hat{ }65, from 0.99×10\hat{ }65 to 1×10\hat{ }66, from 0.99×10\hat{ }66 to 1×10\hat{ }67, from 0.99×10\hat{ }67 to 1×10\hat{ }68, from 0.99×10\hat{ }68 to 1×10\hat{ }69, from 0.99×10\hat{ }69 to 1×10\hat{ }70, from 0.99×10\hat{ }70 to 1×10\hat{ }71, from 0.99×10\hat{ }71 to 1×10\hat{ }72, from 0.99×10\hat{ }72 to 1×10\hat{ }73, from 0.99×10\hat{ }73 to 1×10\hat{ }74, from 0.99×10\hat{ }74 to 1×10\hat{ }75, from 0.99×10\hat{ }75 to 1×10\hat{ }76, from 0.99×10\hat{ }76 to 1×10\hat{ }77, from 0.99×10\hat{ }77 to 1×10\hat{ }78, from 0.99×10\hat{ }78 to 1×10\hat{ }79, from 0.99×10\hat{ }79 to 1×10\hat{ }80, from 0.99×10\hat{ }80 to 1×10\hat{ }81, from 0.99×10\hat{ }81 to 1×10\hat{ }82, from 0.99×10\hat{ }82 to 1×10\hat{ }83, from 0.99×10\hat{ }83 to 1×10\hat{ }84, from 0.99×10\hat{ }84 to 1×10\hat{ }85, from 0.99×10\hat{ }85 to 1×10\hat{ }86, from 0.99×10\hat{ }86 to 1×10\hat{ }87, from 0.99×10\hat{ }87 to 1×10\hat{ }88, from 0.99×10\hat{ }88 to 1×10\hat{ }89, from 0.99×10\hat{ }89 to 1×10\hat{ }90, from 0.99×10\hat{ }90 to 1×10\hat{ }91, from 0.99×10\hat{ }91 to 1×10\hat{ }92, from 0.99×10\hat{ }92 to 1×10\hat{ }93, from 0.99×10\hat{ }93 to 1×10\hat{ }94, from 0.99×10\hat{ }94 to 1×10\hat{ }95, from 0.99×10\hat{ }95 to 1×10\hat{ }96, from 0.99×10\hat{ }96 to 1×10\hat{ }97, from 0.99×10\hat{ }97 to 1×10\hat{ }98, from 0.99×10\hat{ }98 to 1×10\hat{ }99, or from 0.99×10\hat{ }99 to 1×10\hat{ }100 Abeta fragments or molecules.

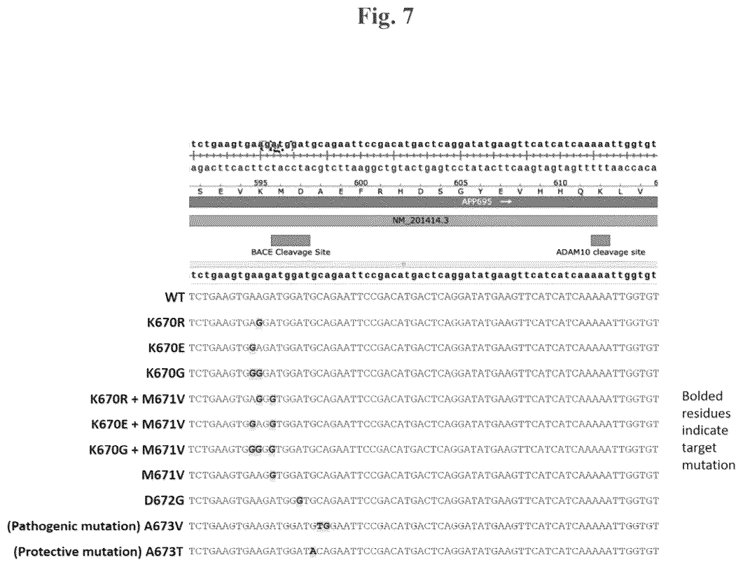

In some cases, the unedited polynucleotide encodes at least a portion of an amyloid precursor protein having at least about 65%, 70%, 75%, 80%, 85%, 90%, 95%, 97%, 98%, or 99% sequence homology to SEQ ID NO: 3-13. An APP mRNA sequence can be targeted. In an embodiment, a specific residue can be targeted utilizing compositions and methods provided herein. Specific residues can comprise point mutations as compared to a wildtype sequence such as that provided in TABLE 2, FIG. 7, and FIG. 8. In some cases, any one of the 3,583 residues of the sequence may be targeted utilizing the compositions and method provided herein. In some cases, a target residue may be located among residues 1 to 100, from 99 to 200, from 199 to 300, from 299 to 400, from 399 to 500, from 499 to 600, from 599 to 700, from 699 to 800, from 799 to 900, from 899 to 1000, from 999 to 1100, from 1099 to 1200, from 1199 to 1300, from 1299 to 1400, from 1399 to 1500, from 1499 to 1600, from 1599 to 1700, from 1699 to 1800, from 1799 to 1900, from 1899 to 2000, from 1999 to 2100, from 2099 to 2200, from 2199 to 2300, from 2299 to 2400, from 2399 to 2500, from 2499 to 2600, from 2599 to 2700, from 2699 to 2800, from 2799 to 2900, from 2899 to 3000, from 2999 to 3100, from 3099 to 3200, from 3199 to 3300, from 3299 to 3400, from 3399 to 3583, or any combination thereof. A target residue may be located at residue 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, and/or 714. In some embodiments, the engineered polynucleotides disclosed herein can target a secretase enzyme cleavage site in APP and edit said cleavage site in order to modulate processing and cleavage of APP by secretase enzymes (e.g., a beta secretase such as BACE1, cathepsin B or Meprin beta). Examples of specific residues that can be targeted by engineered polynucleotide are provided in TABLE 3.

TABLE 2
Exemplary mutations that can be targeted along
with the nearest cleavage site

| # | Mutation | Nearest Cleavage Site |
|---|---|---|
| 1 | K670E | BACE (β-site) |
| 2 | K670R | BACE (β-site) |
| 3 | K670G | BACE (β-site) |
| 4 | K670E + M671V | BACE (β-site) |
| 5 | K670R + M671V | BACE (β-site) |
| 6 | K670G + M671V | BACE (β-site) |
| 7 | M671V | BACE (β-site) |
| 8 | D672G | BACE (β-site) |
| 9 | E682G | BACE (β′-site) |
| 10 | H684R | BACE (β′-site) |
| 11 | K687E | α-secretase |
| 12 | K687R | α-secretase |
| 13 | K687G | α-secretase |
| 14 | A673V | Influences BACE, known |
| (Control) | | pathogenic mutation |
| 15 | A673T | Influences BACE, known |
| (Control) | | protective mutation |

TABLE 3
Exemplary residues in APP polypeptide
that can be targeted

| # | Amino Acid | Residue |
|---|---|---|
| 1 | K670 | 670 |
| 2 | M671 | 671 |
| 3 | D672 | 672 |
| 4 | A673 | 673 |
| 5 | E682 | 682 |
| 6 | H684 | 684 |
| 7 | K687 | 687 |
| 8 | I712 | 712 |
| 9 | T714 | 714 |

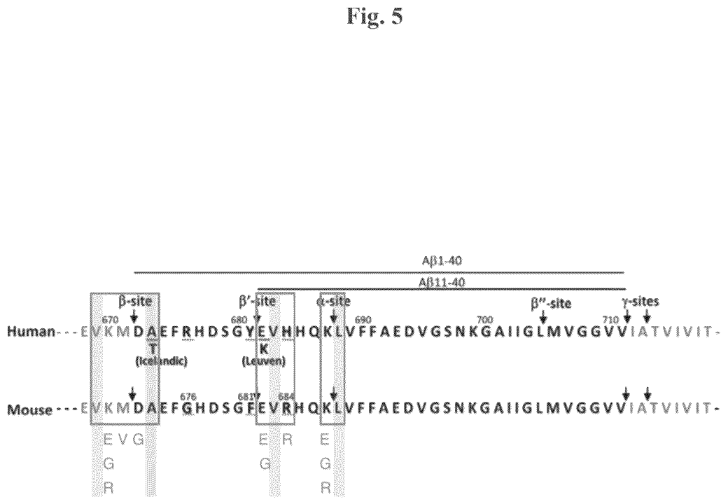

In some cases, the altered susceptibility to protease cleavage comprises a reduced susceptibility by the protease. In some cases, the reduced susceptibility to protease cleavage comprises reduced susceptibility to cleavage at a position cleaved by a beta-secretase. A reduced susceptibility by the protease can be 0.001%, 0.01%, 0.1%, 1%, 10%, 20%, 30%, 40%, 50%, 60%, 70%, 80%, 90%, from 0.00099% to 0.01%, from 0.0099% to 0.1%, from 0.099% to 1%, from 0.99% to 10%, from 9.99% to 20%, from 19.99% to 30%, from 29.99% to 40%, from 39.99% to 50%, from 49.99% to 60%, from 59.99% to 70%, from 69.99% to 80%, or from 79.99% to 90%, of that of a wild-type control. A reduced susceptibility by the protease can also be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% of that of a wild-type control. In some cases, the reduced susceptibility to protease cleavage comprises reduced susceptibility to cleavage at a position cleaved by a Beta-secretase (e.g., BACE1, cathepsin B or Meprin beta). In some cases, the reduced susceptibility to cleavage is at one or more cleavage sites of the amyloid precursor protein. In some cases, the reduced susceptibility to cleavage is at the β-site or the β′-site of the amyloid precursor protein as indicated in FIG. 5. In some cases, the reduced susceptibility to cleavage is at the β-site. For example, upon administration of an engineered guide RNA of the present disclosure, the engineered guide RNA results in ADAR-mediated editing of the β cleavage site in APP, resulting in reduced cleavage of APP by a Beta-secretase (e.g., BACE1, cathepsin B or Meprin beta) at the β cleavage site.

In some cases, the altered susceptibility to protease cleavage comprises an enhanced susceptibility. An enhanced susceptibility by the protease can be 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 76-fold, at least 77-fold, at least 78-fold, at least 79-fold, at least 80-fold, at least 81-fold, at least 82-fold, at least 83-fold, at least 84-fold, at least 85-fold, at least 86-fold, at least 87-fold, at least 88-fold, at least 89-fold, at least 90-fold, at least 91-fold, at least 92-fold, at least 93-fold, at least 94-fold, at least 95-fold, at least 96-fold, at least 97-fold, at least 98-fold, at least 99-fold, at least 100-fold, 1-20-fold, 2-21-fold, 3-22-fold, 4-23-fold, 5-24-fold, 6-25-fold, 7-26-fold, 8-27-fold, 9-28-fold, 10-29-fold, 11-30-fold, 12-31-fold, 13-32-fold, 14-33-fold, 15-34-fold, 16-35-fold, 17-36-fold, 18-37-fold, 19-38-fold, 20-39-fold, 21-40-fold, 22-41-fold, 23-42-fold, 24-43-fold, 25-44-fold, 26-45-fold, 27-46-fold, 28-47-fold, 29-48-fold, 30-49-fold, 31-50-fold, 32-51-fold, 33-52-fold, 34-53-fold, 35-54-fold, 36-55-fold, 37-56-fold, 38-57-fold, 39-58-fold, 40-59-fold, 41-60-fold, 42-61-fold, 43-62-fold, 44-63-fold, 45-64-fold, 46-65-fold, 47-66-fold, 48-67-fold, 49-68-fold, 50-69-fold, 51-70-fold, 52-71-fold, 53-72-fold, 54-73-fold, 55-74-fold, 56-75-fold, 57-76-fold, 58-77-fold, 59-78-fold, 60-79-fold, 61-80-fold, 62-81-fold, 63-82-fold, 64-83-fold, 65-84-fold, 66-85-fold, 67-86-fold, 68-87-fold, 69-88-fold, 70-89-fold, 71-90-fold, 72-91-fold, 73-92-fold, 74-93-fold, 75-94-fold, 76-95-fold, 77-96-fold, 78-97-fold, 79-98-fold, 80-99-fold, or 81-100-fold of that of a wild-type control. In some cases, the enhanced susceptibility to protease cleavage comprises enhanced susceptibility to cleavage at a position cleaved by an α-secretase or γ-secretase. In some cases, the enhanced susceptibility to cleavage is at an α-site of the amyloid precursor protein as indicated in FIG. 5.

In some cases, the altered susceptibility to protease cleavage comprises a reduced susceptibility to cleavage at one cleavage site (e.g., the β-site or the β′-site) and an enhanced susceptibility to cleavage at another cleavage site (e.g., the α-site), wherein the β-site, the β′-site, and the α-site are as indicated in FIG. 5. As such, engineered polynucleotides of the present disclosure may target a β-site to decrease Beta-secretase (e.g., BACE1, cathepsin B or Meprin beta) cleavage. Additionally, or alternatively, engineered polynucleotides of the present disclosure may target an α-site to increase cleavage by an alpha secretase, thereby indirectly reducing Beta-secretase (e.g., BACE1, cathepsin B or Meprin beta) cleavage. Compositions with more than one engineered polynucleotides targeting the α-site and the β-site are contemplated, thereby allowing for multiplexed therapies. In some cases, the modified amyloid precursor protein contains multiple amino acid substitutions. In some cases, the multiple amino acid substitutions are in proximity to different cleavage sites. Proximity or in proximity can mean being separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

In some embodiments, the engineered polynucleotides disclosed herein can target a secretase enzyme cleavage site in APP and edit said cleavage site in order to modulate processing and cleavage of APP by secretase enzymes (e.g., a beta secretase such as BACE1, cathepsin B or Meprin beta). In some cases, the modified amyloid precursor protein comprises a substitution of an amino acid compared to the unedited amyloid precursor protein. The present disclosure provides engineered guide RNAs that target and mediate substitution of the amino acid in the modified APP as compared to unedited APP. Such substitutions are made via ADAR-mediated RNA editing using the disclosed engineered guide RNAs. In some cases, the substitution is at a position in proximity to a cleavage site of the amyloid precursor protein. In some cases, the substitution is at a position up to 2, 3, 4, 5, 6, 7, 8, 9, or 10 amino acids away from the cleavage site. In some cases, the substitution is at a position up to 5 amino acids away from the cleavage site. In some cases, the substitution is at a position up to 3 amino acids away from the cleavage site. In some cases, the substitution is at a position up to 2 amino acids away from the cleavage site. In some cases, the substitution is at the same amino acid position as the cleavage site. In some cases, the substitution is at a position up to 5, 10, 15, 20, or 25 angstroms from the cleavage site. In some cases, the distance from the cleavage site is measured in a folded amyloid precursor protein. In some cases, the cleavage site is the β-site. In some cases, the cleavage site is the β′-site. In some cases, the cleavage site is the α-site. In some cases, the cleavage site is a γ-site. In some cases, the cleavage site is a β-site, β′-site, α-site, γ-site, or any combination thereof. An engineered guide polynucleotide of the present disclosure may facilitate editing of a β-site, β′-site, α-site, γ-site, or any combination thereof by ADAR-mediated editing of an mRNA (pre-mRNA or mRNA) encoding an Abeta polypeptide, wherein the substitution of an amino acid affected by the editing is up to 2, 3, 4, 5, 6, 7, 8, 9, or 10 amino acids from the β-site, β′-site, α-site, γ-site, or any combination thereof.

In some cases, the substitution in the amino acid comprises a substitution that results in a change in charge, hydrophobicity, or polarity of the amino acid, or any combination thereof. In some cases, the substitution in the amino acid comprises a substitution that results in a change in charge of the amino acid. In some cases, the change in charge is from positive to negative, negative to positive, neutral to positive, or neutral to negative. In some cases, the substitution in the amino acid comprises a conservative substitution. In some cases, the substitution in the amino acid comprises a charge neutral substitution. In some cases, the substitution in the amino acid comprises a radical substitution. In some cases, the change in the amino acid comprises a K to E change, a K to R change, a K to G change, an M to V change, a D to G change, an E to G change, an H to R change, or any combination thereof.

In some cases, the substitution in the amino acid is at a position corresponding to position 670, 671, 672, 673, 682, 684, 687, 712, or 714 of the amyloid precursor protein. In some cases, the substitution in the amino acid is at a position corresponding to position 670, 671, 672, 673, 682, 684, 687, 712, or 714 of a polynucleotide sequence encoding for the amyloid precursor protein. In some cases, the substitution in the amino acid is at a position corresponding to position 670, 671, 672, 673, 682, 684, 687, 712, or 714 of the amyloid precursor protein of SEQ ID NO: 2. In some cases, the substitution in the amino acid is at a position corresponding to position 670, 671, 672, 673, 682, 684, 687, 712, or 714 of the amyloid precursor protein of SEQ ID NO: 2 as determined by a Smith-Waterman alignment algorithm. In some cases, the substitution is at a position corresponding to position 670 or 671 of a polynucleotide sequence encoding for an amyloid precursor protein. In some cases, modified amyloid precursor protein comprises a substitution corresponding to position 670 and 671 of a polynucleotide sequence encoding for an amyloid precursor protein. In some cases, the substitution is at a position corresponding to position 670 of a polynucleotide sequence encoding for an amyloid precursor protein. In some cases, the substitution is at a position corresponding to position 671 of a polynucleotide sequence encoding for an amyloid precursor protein.

In some cases, the modified amyloid precursor protein comprises multiple amino acid substitutions. In some cases, the modified amyloid precursor protein comprises an amino acid substitution at a position corresponding to position 670 or 671 of a polynucleotide sequence encoding for the amyloid precursor protein and an additional substitution. In some cases, the modified amyloid precursor protein comprises an amino acid substitution at a position corresponding to position 670 and 671 of a polynucleotide sequence encoding for the amyloid precursor protein and an additional substitution. In some cases, the modified amyloid precursor protein comprises an amino acid substitution at a position corresponding to position 670 of a polynucleotide sequence encoding for the amyloid precursor protein and an additional substitution. In some cases, the modified amyloid precursor protein comprises an amino acid substitution at a position corresponding to position 671 of a polynucleotide sequence encoding for the amyloid precursor protein and an additional substitution. In some cases, the additional amino acid substitution is at a position corresponding to, 670, 671, 672, 673, 682, 684, 687, 712, or 714 of a polynucleotide sequence encoding for the amyloid precursor protein.

In some cases, the amino acid substitution of the modified polynucleotide sequence encoding for amyloid precursor protein comprises K670E, K670R, K670G, M671V, D672G, E682G, H684R, K687R, K687E, or K687G, wherein the position is determined by comparing the sequence of the modified amyloid precursor protein to the amyloid precursor protein of SEQ ID NO: 2 as determined by a Smith-Waterman alignment algorithm. In some cases, the amyloid precursor protein is the amyloid precursor protein of SEQ ID NO: 2. In some cases, the amino acid substitution comprises K670G or M671V. In some cases, the amino acid substitution comprises K670G. In some cases, the amino acid substitution comprises M671V.

In some cases, the engineered guide RNA facilitates an edit in a polynucleotide at least partially encoding for amyloid precursor protein. In some cases, the engineered guide RNA is at least partially complementary to at least a portion of the polynucleotide. In some cases, at least a portion of the engineered polynucleotide forms an at least partially double-stranded oligonucleotide with the targeted polynucleotide/RNA. This at least partially double-stranded oligonucleotide may also be referred to as a double-stranded RNA (dsRNA) substrate. For example, the engineered guide RNA may have a sequence that hybridizes to a sequence of the polynucleotide encoding for APP. The double-stranded oligonucleotide or dsRNA substrate refers to the sequence and structure formed upon hybridization of the sequence of the engineered guide RNA to the sequence of the polynucleotide encoding for APP. The double stranded oligonucleotide may comprise a single base mismatch between the sequence of the polynucleotide and the corresponding sequence of the engineered guide RNA. The single base mismatch may be at the target base to be edited, for example the adenosine in the target polynucleotide to be edited by an RNA editing protein. In some cases, the engineered guide RNA associates with an editing protein (e.g., an RNA editing protein such as ADAR). In some cases, the editing protein in association with the at least partially double-stranded oligonucleotide is capable of recruiting the editing protein. The RNA editing protein (e.g., ADAR) may comprise one or two polypeptide chains. Two polypeptide chains may be identical. Two polypeptide chains may not be identical. Two ADAR polypeptide chains may form a homodimer. Two ADAR polypeptide chains may form a heterodimer. Upon formation of the at least partially double stranded oligonucleotide, a first monomer (one of the polypeptide chains) of the homodimer or heterodimer may associate with the at least partially double stranded oligonucleotide followed by a second monomer (the other polypeptide chain) of the homodimer or heterodimer. In some embodiments, a pre-formed homodimer or heterodimer associate with the at least partially double stranded oligonucleotide. In some cases, a monomer of ADAR may associate with the at least partially double stranded oligonucleotide. In other cases, a first monomer and a second monomer may bind the at least partially double stranded oligonucleotide independently. Upon binding to the at least partially double stranded oligonucleotide, the first and second monomer may form a homodimer or a heterodimer. In some cases, the editing protein, once bound to an engineered polynucleotide provided herein, modifies a base of a nucleotide in the targeted polynucleotide/RNA. In particular, the editing protein is an RNA editing protein and modifies an RNA base of an RNA nucleotide in a target RNA. In some cases, modifying the base produces a modified polynucleotide at least partially encoding the modified amyloid precursor protein.

In some cases, the engineered guide RNAs disclosed herein facilitate editing (e.g., via ADAR) of a base of a nucleotide comprised in a codon which encodes an amino acid in proximity to a secretase cleavage site of the amyloid precursor protein. The amino acid in proximity to the secretase cleavage site of the amyloid precursor protein can be any of the amino acids provided herein. In some cases, the base of the nucleotide is comprised in a codon which encodes an amino acid in proximity to: an alpha-secretase cleavage site, a beta-secretase cleavage site (e.g., a ®- or ®′-cleavage site), or a gamma-secretase cleavage site of the amyloid precursor protein, or any combination thereof. In some embodiments, the base of the nucleotide is comprised in a codon which encodes an amino acid in proximity to the ®- or ®′-cleavage site. In some cases, the engineered guide RNAs disclosed herein facilitate editing (e.g., via ADAR) of a base of a nucleotide comprised in a codon which encodes an amino acid in proximity to a position corresponding to position 670, 671, 672, 673, 682, 684, 687, 712, or 714 of a sequence encoding for an amyloid precursor. In some cases, the engineered guide RNAs disclosed herein facilitate editing (e.g., via ADAR) of a base of a nucleotide comprised in a codon which encodes an amino acid in proximity to a position corresponding to position 670, 671, 672, 673, 682, 684, 687, 712, or 714 of the amyloid precursor protein of SEQ ID NO: 2, as determined by a Smith-Waterman alignment algorithm. In some cases, the base of the nucleotide is comprised in a codon which encodes an amino acid at a position corresponding to position 670, 671, 672, 673, 682, 684, 687, 712, or 714 of the amyloid precursor protein of SEQ ID NO: 2 as determined by a Smith-Waterman alignment algorithm. The base of the nucleotide may be comprised in a codon encoding an amino acid at a position corresponding to position 670 of the amyloid precursor protein of SEQ ID NO: 2, as determined by a Smith-Waterman alignment algorithm. The base of the nucleotide may be comprised in a codon encoding an amino acid at a position corresponding to position 671 of the amyloid precursor protein of SEQ ID NO: 2, as determined by a Smith-Waterman alignment algorithm. In some cases, modifying the base of the nucleotide results in a change in the amino acid sequence of the modified amyloid precursor protein translated from the modified RNA as compared to an amyloid precursor protein translated from the unedited RNA. The change in the amino acid can be any of the amino acid substitutions provided herein.

In some cases, the editing protein in association with the engineered guide RNA and the polynucleotide modifies a second base on a second nucleotide of the polynucleotide. In some embodiments, the first and second nucleotide can belong to the same codon. In some cases, the first and second nucleotide can belong to two different codons. In some instances, the first nucleotide and the second nucleotide are consecutive.

In some cases, the engineered guide RNAs of the present disclosure modify a polynucleotide encoding for APP via an RNA editing protein (e.g., ADAR). Said modification of the base of a nucleotide in a sequence of the polynucleotide is a chemical modification. In some cases, the chemical modification is a deamination. In some cases, the modification results in the cell's translational machinery reading the original, unedited base as a different base. In some cases, the base is an adenosine. In some cases, the base is an adenosine and the engineered guide RNAs of the present disclosure facilitate modification of the adenosine to inosine. In some cases, the base is an adenosine and the engineered guide RNAs of the present disclosure recruit ADAR, which modifies the adenosine to inosine.

In some cases, the polynucleotide at least partially encoding the amyloid precursor protein is mRNA or pre-mRNA. In some cases, the polynucleotide is mRNA. In some cases, the polynucleotide is pre-mRNA. In some cases, the polynucleotide at least partially encoding the amyloid precursor protein is DNA. The engineered guide RNAs of the present disclosure, thus, may facilitate editing of mRNA or pre-mRNA by recruiting an RNA editing protein such as ADAR to the mRNA or the pre-mRNA and modifying adenosines to inosines. The engineered guide RNAs of the present disclosure, thus, may facilitate editing of mRNA by recruiting an RNA editing protein such as ADAR to the mRNA and modifying adenosines to inosines. The engineered guide RNAs of the present disclosure, thus, may facilitate editing of pre-mRNA by recruiting an RNA editing protein such as ADAR to the pre-mRNA and modifying adenosines to inosines.

In some cases, the modified amyloid precursor protein encoded by the modified polynucleotide as facilitated by the engineered guide RNA does not otherwise inhibit protease cleavage of other substrates even as proteases have less activity on the modified amyloid precursor protein compared to the unedited amyloid precursor protein. For example, while engineered guide RNAs of the present disclosure may facilitate an edit in APP resulting in reduced cleavage at the Beta-secretase (e.g., BACE1, cathepsin B or Meprin beta) cleavage site cleavage site within APP, Beta-secretase (e.g., BACE1, cathepsin B or Meprin beta) cleaving activity may not be diminished on other endogenous targets of a Beta-secretase (e.g., BACE1, cathepsin B or Meprin beta). This can be measured by ascertaining the amount of cleavage metabolites of other such endogenous targets of a Beta-secretase (e.g., BACE1, cathepsin B or Meprin beta) in a) a cell expressing the modified amyloid precursor protein, and b) a cell expressing the unedited amyloid precursor protein and comparing the values. The values can optionally be normalized to expression of the endogenous protein in each cell, or to another suitable marker. Measurement of the other endogenous targets of a Beta-secretase (e.g., BACE1, cathepsin B or Meprin beta) and cleavage metabolites can be performed by a variety of in vitro assays, including without limitation enzyme-linked immunosorbent assays (ELISAs) or mass spectrometry techniques, such as LC-MS or MALDI. Non-limiting examples of other endogenous targets of a Beta-secretase, which can be suitable for measurement of metabolites indicative of protease activity amyloid-like protein 1 (APLP1), amyloid-like protein 2 (APLP2), Contactin 2, Jagged 1, neural cell adhesion molecule L1 (CHL1), Neurexin 1α, Neurexin 3β, neuregulin 1 (NRG1), seizure related protein 6 (SEZ6), seizure related protein 6 precursor protein (SEZ6L), a β (β1-4) Auxiliary subunit of the voltage-gated sodium ion channel (VGSC) subtype Nav1, VGSC Accessory Subunits KCNE1 or KCNE2, a functional portion of any of these, or any combination of thereof. In some cases, the endogenous substrate examined to determine the extent to which beta secretase activity has been inhibited is NRG1, SEZ6, or CHL1.

In some cases, the engineered guide RNA capable of facilitating a modification on a base of a nucleotide comprised in a polynucleotide encoding at least a portion of the amyloid precursor protein produces a modified amyloid precursor protein comprising at least one amino acid substitution compared to an unedited polynucleotide. In some cases, the modified amyloid precursor protein (containing modifications of the cleavage site cleavage site) produces a lower amount of Abeta 40, Abeta 42 when cleaved by a Beta-secretase (e.g., BACE1, cathepsin B or Meprin beta), or both when expressed in a cell compared to a corresponding cell expressing the unedited amyloid precursor protein for a comparable period of time. In some cases, the amount of Abeta 40, Abeta 42, or both is measured by an Abeta 40 or Abeta 42 ELISA, or both. In some cases, the modified amyloid precursor protein produces an increased amount of secreted ectodomain APP alpha (sAPPa) when expressed in a cell compared to the unedited amyloid precursor protein for a comparable period of time. In some cases, the amount of sAPPa or the beta-COOH-terminal fragment is measured by a sAPPa ELISA. In some cases, amounts of Abeta 40, Abeta 42, or both, and amounts of sAPPa are measured. The engineered guide RNAs of the present disclosure may be used to facilitate an edit at a Beta-secretase (e.g., BACE1, cathepsin B or Meprin beta) cleavage site in APP, resulting in a decreased production of Abeta 40 and Abeta42 metabolites. In some embodiments, the engineered guide RNAs and methods of using said engineered guide RNAs disclosed herein can result in at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 76-fold, at least 77-fold, at least 78-fold, at least 79-fold, at least 80-fold, at least 81-fold, at least 82-fold, at least 83-fold, at least 84-fold, at least 85-fold, at least 86-fold, at least 87-fold, at least 88-fold, at least 89-fold, at least 90-fold, at least 91-fold, at least 92-fold, at least 93-fold, at least 94-fold, at least 95-fold, at least 96-fold, at least 97-fold, at least 98-fold, at least 99-fold, at least 100-fold, 1-20-fold, 2-21-fold, 3-22-fold, 4-23-fold, 5-24-fold, 6-25-fold, 7-26-fold, 8-27-fold, 9-28-fold, 10-29-fold, 11-30-fold, 12-31-fold, 13-32-fold, 14-33-fold, 15-34-fold, 16-35-fold, 17-36-fold, 18-37-fold, 19-38-fold, 20-39-fold, 21-40-fold, 22-41-fold, 23-42-fold, 24-43-fold, 25-44-fold, 26-45-fold, 27-46-fold, 28-47-fold, 29-48-fold, 30-49-fold, 31-50-fold, 32-51-fold, 33-52-fold, 34-53-fold, 35-54-fold, 36-55-fold, 37-56-fold, 38-57-fold, 39-58-fold, 40-59-fold, 41-60-fold, 42-61-fold, 43-62-fold, 44-63-fold, 45-64-fold, 46-65-fold, 47-66-fold, 48-67-fold, 49-68-fold, 50-69-fold, 51-70-fold, 52-71-fold, 53-72-fold, 54-73-fold, 55-74-fold, 56-75-fold, 57-76-fold, 58-77-fold, 59-78-fold, 60-79-fold, 61-80-fold, 62-81-fold, 63-82-fold, 64-83-fold, 65-84-fold, 66-85-fold, 67-86-fold, 68-87-fold, 69-88-fold, 70-89-fold, 71-90-fold, 72-91-fold, 73-92-fold, 74-93-fold, 75-94-fold, 76-95-fold, 77-96-fold, 78-97-fold, 79-98-fold, 80-99-fold, or 81-100-fold decrease in the protein level of Abeta 40, 42, or both, as compared to that generated upon Beta-secretase (e.g., BACE1, cathepsin B or Meprin beta) cleavage of unedited APP.

Methods of mRNA base editing can result in at least partially reducing an amount of peptide bond cleavage of a protein (such as APP). Methods can include altering a cleavage site, such as a BACE cleavage site, including the ®-site, the ®′-site, or a combination thereof. Methods can include altering BACE-mediated degradation of APP. A reduced amount of peptide bond cleavage can be adjacent to an alanine, a cysteine, an aspartic acid, a glutamic acid, a phenylalanine, a glycine, a histidine, an isoleucine, a lysine, a leucine, a methionine, an asparagine, a proline, a glutamine, an arginine, a serine, a threonine, a tryptophan, a tyrosine, or a valine or the protein. The reduced amount of peptide bond cleavage can be adjacent to a methionine or an aspartate of the protein.

Methods to monitor the efficacy of the engineered nucleotide targeting APP can comprise in vitro demonstration of molecular efficiency in APP base editing, reduction in Abeta 1, Abeta 2, Abeta 3, Abeta 4, Abeta 5, Abeta 6, Abeta 7, Abeta 8, Abeta 9, Abeta 10, Abeta 11, Abeta 12, Abeta 13, Abeta 14, Abeta 15, Abeta 16, Abeta 17, Abeta 18, Abeta 19, Abeta 20, Abeta 21, Abeta 22, Abeta 23, Abeta 24, Abeta 25, Abeta 26, Abeta 27, Abeta 28, Abeta 29, Abeta 30, Abeta 31, Abeta 32, Abeta 33, Abeta 34, Abeta 35, Abeta 36, Abeta 37, Abeta 38, Abeta 39, Abeta 40, Abeta 41, Abeta 42 production using cells naturally expressing sufficient levels of APP, model cell lines where APP is knocked out, cells transfected with WT APP, or other cell types including, but not limited to human neuroblastoma cells, human iPSCs and derived cell types, human neural progenitor cells and derived cell types, LUHMES cells, NTera-2 cells, and/or primary cells cultured from mice containing a humanized APP sequence. Model cell lines include, but are not limited to, 293 cells, COS cells, HeLa cells, Vero cells, 3T3 mouse fibroblasts, C3H10T1/2 fibroblasts, CHO cells, and the like. Exemplary host cells include, without limitation, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

Abeta oligomers can initiate pathogenesis in Alzheimer's disease, however, their suppression cannot be sufficient to reduce disease progression. Targeting insoluble Abeta and Abeta aggregates can ignore the toxicity of soluble Abeta and/or C-terminal fragments, which can be highly toxic as well. Expectations of an effect with only Abeta reduction can ignore other identified pathogenic drivers (e.g., p-Tau, alpha-synuclein).

In some embodiments, the editing of a base of the APP mRNA results in a decreased gene translation of the APP polypeptide. In other cases, the editing of a base of the 5′UTR of the APP mRNA results in a decreased gene translation of the APP polypeptide. The decreased gene translation of the APP polypeptide can be measure by an in vitro assay. Such in vitro assay can comprise an in vitro translation assay. An in vitro translation assay can comprise a cell extract. A cell extract can comprise rabbit reticulocyte lysate, wheat germ extract, insect cells, yeast Kluyveromyces, or E. coli cell-free extract. An in vitro translation assay can comprise mixing a cell extract with a nucleic acid template, ATP, and amino acids. A nucleic acid template can comprise a mRNA template or a cDNA template. A nucleic acid template can comprise a mRNA sequence listed in TABLE 1 or 13. A nucleic acid template can comprise a cDNA sequence complementary to the mRNA sequence listed in TABLE 1 or 13. When using an in vitro translation system with a cDNA template, the cDNA can be converted to a mRNA by in vitro transcription. A cDNA can be maintained in a circular vector. A cDNA can be maintained as a linear sequence. Therefore, a multi-targeted combination therapy, such as described in the compositions and methods herein, can provide the necessary disease modification in Alzheimer's disease.

### Tau

Tau proteins (Tau-p) are encoded by six mRNA isoforms of Tau MAPT. Tau-p is a microtubule-binding protein, important for microtubule stability and transport. It is primarily expressed in the neurons of the CNS. The aggregation of hyperphosphorylated mutant Tau proteins into neurofibrillary tangles (NFTs) in the human brain causes a group of neurodegenerative diseases named Tauopathies, including Alzheimer's Disease. Proteolytic Tau cleavage fragments (that can be formed by calpain-mediated proteolysis, activated downstream of Abeta production) can also be directly neurotoxic. Therefore, a multiplex strategy to substantially reduce Tau formation can be important in effectively treating neurodegenerative diseases.

In an embodiment, a specific residue can be targeted utilizing compositions and methods provided herein. Complete Tau mRNA sequence are shown in TABLE 4. In some cases, a target residue can be any one position of the 6,644 residues of the sequence may be targeted utilizing the compositions and method provided herein. In some cases, a target residue may be located among residues from 1 to 100, from 99 to 200, from 199 to 300, from 299 to 400, from 399 to 500, from 499 to 600, from 599 to 700, from 699 to 800, from 799 to 900, from 899 to 1000, from 999 to 1100, from 1099 to 1200, from 1199 to 1300, from 1299 to 1400, from 1399 to 1500, from 1499 to 1600, from 1599 to 1700, from 1699 to 1800, from 1799 to 1900, from 1899 to 2000, from 1999 to 2100, from 2099 to 2200, from 2199 to 2300, from 2299 to 2400, from 2399 to 2500, from 2499 to 2600, from 2599 to 2700, from 2699 to 2800, from 2799 to 2900, from 2899 to 3000, from 2999 to 3100, from 3099 to 3200, from 3199 to 3300, from 3299 to 3400, from 3399 to 3500, from 3499 to 3600, from 3599 to 3700, from 3699 to 3800, from 3799 to 3900, from 3899 to 4000, from 3999 to 4100, from 4099 to 4200, from 4199 to 4300, from 4299 to 4400, from 4399 to 4500, from 4499 to 4600, from 4599 to 4700, from 4699 to 4800, from 4799 to 4900, from 4899 to 5000, from 4999 to 5100, from 5099 to 5200, from 5199 to 5300, from 5299 to 5400, from 5399 to 5500, from 5499 to 5600, from 5599 to 5700, from 5699 to 5800, from 5799 to 5900, from 5899 to 6000, from 5999 to 6100, from 6099 to 6200, from 6199 to 6300, from 6299 to 6400, from 6399 to 6444, or any combination thereof of the Tau mRNA.

TABLE 4
Human MAPT mRNA Isoform Sequences. Sequences obtained
from NCBI MAPT gene ID: 4137; Assembly GRCh38.p13
(GCF_000001405.39); NC_000017.11 (45894538 . . . 46028334)

| SEQ | | |
|---|---|---|
| ID | | |
| NO | Isoform | mRNA Sequence |
| 16 | 1 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GAUGUGACAGCACCCUUAGUGGAUGAGGGAGCUCCCGGCAAGCAGG |
| | | CUGCCGCGCAGCCCCACACGGAGAUCCCAGAAGGAACCACAGCUGA |
| | | AGAAGCAGGCAUUGGAGACACCCCCAGCCUGGAAGACGAAGCUGCU |
| | | GGUCACGUGACCCAAGAGCCUGAAAGUGGUAAGGUGGUCCAGGAAG |
| | | GCUUCCUCCGAGAGCCAGGCCCCCCAGGUCUGAGCCACCAGCUCAU |
| | | GUCCGGCAUGCCUGGGGCUCCCCUCCUGCCUGAGGGCCCCAGAGAG |
| | | GCCACACGCCAACCUUCGGGGACAGGACCUGAGGACACAGAGGGCG |
| | | GCCGCCACGCCCCUGAGCUGCUCAAGCACCAGCUUCUAGGAGACCU |
| | | GCACCAGGAGGGGCCGCCGCUGAAGGGGGCAGGGGGCAAAGAGAGG |
| | | CCGGGGAGCAAGGAGGAGGUGGAUGAAGACCGCGACGUCGAUGAGU |
| | | CCUCCCCCCAAGACUCCCCUCCCUCCAAGGCCUCCCCAGCCCAAGAU |
| | | GGGCGGCCUCCCCAGACAGCCGCCAGAGAAGCCACCAGCAUCCCAG |
| | | GCUUCCCAGCGGAGGGUGCCAUCCCCCUCCCUGUGGAUUUCCUCUC |
| | | CAAAGUUUCCACAGAGAUCCCAGCCUCAGAGCCCGACGGGCCCAGU |
| | | GUAGGGCGGGCCAAAGGGCAGGAUGCCCCCCUGGAGUUCACGUUUC |
| | | ACGUGGAAAUCACACCCAACGUGCAGAAGGAGCAGGCGCACUCGGA |
| | | GGAGCAUUUGGGAAGGGCUGCAUUUCCAGGGGCCCCUGGAGAGGGG |
| | | CCAGAGGCCCGGGGCCCCUCUUUGGGAGAGGACACAAAAGAGGCUG |
| | | ACCUUCCAGAGCCCUCUGAAAAGCAGCCUGCUGCUGCUCCGCGGGG |
| | | GAAGCCCGUCAGCCGGGUCCCUCAACUCAAAGCUCGCAUGGUCAGU |
| | | AAAAGCAAAGACGGGACUGGAAGCGAUGACAAAAAAGCCAAGACAU |
| | | CCACACGUUCCUCUGCUAAAACCUUGAAAAAUAGGCCUUGCCUUAG |
| | | CCCCAAACACCCCACUCCUGGUAGCUCAGACCCUCUGAUCCAACCCU |
| | | CCAGCCCUGCUGUGUGCCCAGAGCCACCUUCCUCUCCUAAAUACGU |
| | | CUCUUCUGUCACUUCCCGAACUGGCAGUUCUGGAGCAAAGGAGAUG |
| | | AAACUCAAGGGGGCUGAUGGUAAAACGAAGAUCGCCACACCGCGGG |
| | | GAGCAGCCCCUCCAGGCCAGAAGGGCCAGGCCAACGCCACCAGGAU |
| | | UCCAGCAAAAACCCCGCCCGCUCCAAAGACACCACCCAGCUCUGGUG |
| | | AACCUCCAAAAUCAGGGGAUCGCAGCGGCUACAGCAGCCCCGGCUC |
| | | CCCAGGCACUCCCGGCAGCCGCUCCCGCACCCCGUCCCUUCCAACCC |
| | | CACCCACCCGGGAGCCCAAGAAGGUGGCAGUGGUCCGUACUCCACC |
| | | CAAGUCGCCGUCUUCCGCCAAGAGCCGCCUGCAGACAGCCCCCGUGC |
| | | CCAUGCCAGACCUGAAGAAUGUCAAGUCCAAGAUCGGCUCCACUGA |
| | | GAACCUGAAGCACCAGCCGGGAGGCGGGAAGGUGCAGAUAAUUAAU |
| | | AAGAAGCUGGAUCUUAGCAACGUCCAGUCCAAGUGUGGCUCAAAGG |
| | | AUAAUAUCAAACACGUCCCGGGAGGCGGCAGUGUGCAAAUAGUCUA |
| | | CAAACCAGUUGACCUGAGCAAGGUGACCUCCAAGUGUGGCUCAUUA |
| | | GGCAACAUCCAUCAUAAACCAGGAGGUGGCCAGGUGGAAGUAAAAU |
| | | CUGAGAAGCUUGACUUCAAGGACAGAGUCCAGUCGAAGAUUGGGUC |
| | | CCUGGACAAUAUCACCCACGUCCCUGGCGGAGGAAAUAAAAAGAUU |
| | | GAAACCCACAAGCUGACCUUCCGCGAGAACGCCAAAGCCAAGACAG |
| | | ACCACGGGGCGGAGAUCGUGUACAAGUCGCCAGUGGUGUCUGGGGA |
| | | CACGUCUCCACGGCAUCUCAGCAAUGUCUCCUCCACCGGCAGCAUC |
| | | GACAUGGUAGACUCGCCCCAGCUCGCCACGCUAGCUGACGAGGUGU |
| | | CUGCCUCCCUGGCCAAGCAGGGUUUGUGAUCAGGCCCCUGGGGCGG |
| | | UCAAUAAUUGUGGAGAGGAGAGAAUGAGAGAGUGUGGAAAAAAAA |
| | | AGAAUAAUGACCCGGCCCCCGCCCUCUGCCCCCAGCUGCUCCUCGCA |
| | | GUUCGGUUAAUUGGUUAAUCACUUAACCUGCUUUUGUCACUCGGCU |
| | | UUGGCUCGGGACUUCAAAAUCAGUGAUGGGAGUAAGAGCAAAUUU |
| | | CAUCUUUCCAAAUUGAUGGGUGGGCUAGUAAUAAAAUAUUUAAAA |
| | | AAAAACAUUCAAAAACAUGGCCACAUCCAACAUUUCCUCAGGCAAU |
| | | UCCUUUUGAUUCUUUUUUCUUCCCCCUCCAUGUAGAAGAGGGAGAA |
| | | GGAGAGGCUCUGAAAGCUGCUUCUGGGGGAUUUCAAGGGACUGGG |
| | | GGUGCCAACCACCUCUGGCCCUGUUGUGGGGGUGUCACAGAGGCAG |
| | | UGGCAGCAACAAAGGAUUUGAAACUUGGUGUGUUCGUGGAGCCACA |
| | | GGCAGACGAUGUCAACCUUGUGUGAGUGUGACGGGGGUUGGGGUG |
| | | GGGCGGGAGGCCACGGGGGAGGCCGAGGCAGGGGCUGGGCAGAGGG |
| | | GAGAGGAAGCACAAGAAGUGGGAGUGGGAGAGGAAGCCACGUGCU |
| | | GGAGAGUAGACAUCCCCCUCCUUGCCGCUGGGAGAGCCAAGGCCUA |
| | | UGCCACCUGCAGCGUCUGAGCGGCCGCCUGUCCUUGGUGGCCGGGG |
| | | GUGGGGGCCUGCUGUGGGUCAGUGUGCCACCCUCUGCAGGGCAGCC |
| | | UGUGGGAGAAGGGACAGCGGGUAAAAAGAGAAGGCAAGCUGGCAG |
| | | GAGGGUGGCACUUCGUGGAUGACCUCCUUAGAAAAGACUGACCUUG |
| | | AUGUCUUGAGAGCGCUGGCCUCUUCCUCCCUCCCUGCAGGGUAGGG |
| | | GGCCUGAGUUGAGGGGCUUCCCUCUGCUCCACAGAAACCCUGUUUU |
| | | AUUGAGUUCUGAAGGUUGGAACUGCUGCCAUGAUUUUGGCCACUUU |
| | | GCAGACCUGGGACUUUAGGGCUAACCAGUUCUCUUUGUAAGGACUU |
| | | GUGCCUCUUGGGAGACGUCCACCCGUUUCCAAGCCUGGGCCACUGG |
| | | CAUCUCUGGAGUGUGUGGGGGUCUGGGAGGCAGGUCCCGAGCCCCC |
| | | UGUCCUUCCCACGGCCACUGCAGUCACCCCGUCUGCGCCGCUGUGCU |
| | | GUUGUCUGCCGUGAGAGCCCAAUCACUGCCUAUACCCCUCAUCACA |
| | | CGUCACAAUGUCCCGAAUUCCCAGCCUCACCACCCCUUCUCAGUAA |
| | | UGACCCUGGUUGGUUGCAGGAGGUACCUACUCCAUACUGAGGGUGA |
| | | AAUUAAGGGAAGGCAAAGUCCAGGCACAAGAGUGGGACCCCAGCCU |
| | | CUCACUCUCAGUUCCACUCAUCCAACUGGGACCCUCACCACGAAUC |
| | | UCAUGAUCUGAUUCGGUUCCCUGUCUCCUCCUCCCGUCACAGAUGU |
| | | GAGCCAGGGCACUGCUCAGCUGUGACCCUAGGUGUUUCUGCCUUGU |
| | | UGACAUGGAGAGAGCCCUUUCCCCUGAGAAGGCCUGGCCCCUUCCU |
| | | GUGCUGAGCCCACAGCAGCAGGCUGGGUGUCUUGGUUGUCAGUGGU |
| | | GGCACCAGGAUGGAAGGGCAAGGCACCCAGGGCAGGCCCACAGUCC |
| | | CGCUGUCCCCCACUUGCACCCUAGCUUGUAGCUGCCAACCUCCCAGA |
| | | CAGCCCAGCCCGCUGCUCAGCUCCACAUGCAUAGUAUCAGCCCUCCA |
| | | CACCCGACAAAGGGGAACACACCCCCUUGGAAAUGGUUCUUUUCCC |
| | | CCAGUCCCAGCUGGAAGCCAUGCUGUCUGUUCUGCUGGAGCAGCUG |
| | | AACAUAUACAUAGAUGUUGCCCUGCCCUCCCCAUCUGCACCCUGUU |
| | | GAGUUGUAGUUGGAUUUGUCUGUUUAUGCUUGGAUUCACCAGAGU |
| | | GACUAUGAUAGUGAAAAGAAAAAAAAAAAAAAAAAAGGACGCAUG |
| | | UAUCUUGAAAUGCUUGUAAAGAGGUUUCUAACCCACCCUCACGAGG |
| | | UGUCUCUCACCCCCACACUGGGACUCGUGUGGCCUGUGUGGUGCCA |
| | | CCCUGCUGGGGCCUCCCAAGUUUUGAAAGGCUUUCCUCAGCACCUG |
| | | GGACCCAACAGAGACCAGCUUCUAGCAGCUAAGGAGGCCGUUCAGC |
| | | UGUGACGAAGGCCUGAAGCACAGGAUUAGGACUGAAGCGAUGAUG |
| | | UCCCCUUCCCUACUUCCCCUUGGGGCUCCCUGUGUCAGGGCACAGA |
| | | CUAGGUCUUGUGGCUGGUCUGGCUUGCGGCGCGAGGAUGGUUCUCU |
| | | CUGGUCAUAGCCCGAAGUCUCAUGGCAGUCCCAAAGGAGGCUUACA |
| | | ACUCCUGCAUCACAAGAAAAAGGAAGCCACUGCCAGCUGGGGGGAU |
| | | CUGCAGCUCCCAGAAGCUCCGUGAGCCUCAGCCACCCCUCAGACUG |
| | | GGUUCCUCUCCAAGCUCGCCCUCUGGAGGGGCAGCGCAGCCUCCCA |
| | | CCAAGGGCCCUGCGACCACAGCAGGGAUUGGGAUGAAUUGCCUGUC |
| | | CUGGAUCUGCUCUAGAGGCCCAAGCUGCCUGCCUGAGGAAGGAUGA |
| | | CUUGACAAGUCAGGAGACACUGUUCCCAAAGCCUUGACCAGAGCAC |
| | | CUCAGCCCGCUGACCUUGCACAAACUCCAUCUGCUGCCAUGAGAAA |
| | | AGGGAAGCCGCCUUUGCAAAACAUUGCUGCCUAAAGAAACUCAGCA |
| | | GCCUCAGGCCCAAUUCUGCCACUUCUGGUUUGGGUACAGUUAAAGG |
| | | CAACCCUGAGGGACUUGGCAGUAGAAAUCCAGGGCCUCCCCUGGGG |
| | | CUGGCAGCUUCGUGUGCAGCUAGAGCUUUACCUGAAAGGAAGUCUC |
| | | UGGGCCCAGAACUCUCCACCAAGAGCCUCCCUGCCGUUCGCUGAGU |
| | | CCCAGCAAUUCUCCUAAGUUGAAGGGAUCUGAGAAGGAGAAGGAAA |
| | | UGUGGGGUAGAUUUGGUGGUGGUUAGAGAUAUGCCCCCCUCAUUAC |
| | | UGCCAACAGUUUCGGCUGCAUUUCUUCACGCACCUCGGUUCCUCUU |
| | | CCUGAAGUUCUUGUGCCCUGCUCUUCAGCACCAUGGGCCUUCUUAU |
| | | ACGGAAGGCUCUGGGAUCUCCCCCUUGUGGGGCAGGCUCUUGGGGC |
| | | CAGCCUAAGAUCAUGGUUUAGGGUGAUCAGUGCUGGCAGAUAAAU |
| | | UGAAAAGGCACGCUGGCUUGUGAUCUUAAAUGAGGACAAUCCCCCC |
| | | AGGGCUGGGCACUCCUCCCCUCCCCUCACUUCUCCCACCUGCAGAGC |
| | | CAGUGUCCUUGGGUGGGCUAGAUAGGAUAUACUGUAUGCCGGCUCC |
| | | UUCAAGCUGCUGACUCACUUUAUCAAUAGUUCCAUUUAAAUUGACU |
| | | UCAGUGGUGAGACUGUAUCCUGUUUGCUAUUGCUUGUUGUGCUAU |
| | | GGGGGGAGGGGGGAGGAAUGUGUAAGAUAGUUAACAUGGGCAAAG |
| | | GGAGAUCUUGGGGUGCAGCACUUAAACUGCCUCGUAACCCUUUUCA |
| | | UGAUUUCAACCACAUUUGCUAGAGGGAGGGAGCAGCCACGGAGUUA |
| | | GAGGCCCUUGGGGUUUCUCUUUUCCACUGACAGGCUUUCCCAGGCA |
| | | GCUGGCUAGUUCAUUCCCUCCCCAGCCAGGUGCAGGCGUAGGAAUA |
| | | UGGACAUCUGGUUGCUUUGGCCUGCUGCCCUCUUUCAGGGGUCCUA |
| | | AGCCCACAAUCAUGCCUCCCUAAGACCUUGGCAUCCUUCCCUCUAA |
| | | GCCGUUGGCACCUCUGUGCCACCUCUCACACUGGCUCCAGACACAC |
| | | AGCCUGUGCUUUUGGAGCUGAGAUCACUCGCUUCACCCUCCUCAUC |
| | | UUUGUUCUCCAAGUAAAGCCACGAGGUCGGGGCGAGGGCAGAGGUG |
| | | AUCACCUGCGUGUCCCAUCUACAGACCUGCAGCUUCAUAAAACUUC |
| | | UGAUUUCUCUUCAGCUUUGAAAAGGGUUACCCUGGGCACUGGCCUA |
| | | GAGCCUCACCUCCUAAUAGACUUAGCCCCAUGAGUUUGCCAUGUUG |
| | | AGCAGGACUAUUUCUGGCACUUGCAAGUCCCAUGAUUUCUUCGGUA |
| | | AUUCUGAGGGUGGGGGGAGGGACAUGAAAUCAUCUUAGCUUAGCU |
| | | UUCUGUCUGUGAAUGUCUAUAUAGUGUAUUGUGUGUUUUAACAAA |
| | | UGAUUUACACUGACUGUUGCUGUAAAAGUGAAUUUGGAAAUAAAG |
| | | UUAUUACUCUGAUUAAA |
| 17 | 2 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GAUGUGACAGCACCCUUAGUGGAUGAGGGAGCUCCCGGCAAGCAGG |
| | | CUGCCGCGCAGCCCCACACGGAGAUCCCAGAAGGAACCACAGCUGA |
| | | AGAAGCAGGCAUUGGAGACACCCCCAGCCUGGAAGACGAAGCUGCU |
| | | GGUCACGUGACCCAAGCUCGCAUGGUCAGUAAAAGCAAAGACGGGA |
| | | CUGGAAGCGAUGACAAAAAAGCCAAGGGGGCUGAUGGUAAAACGA |
| | | AGAUCGCCACACCGCGGGGAGCAGCCCCUCCAGGCCAGAAGGGCCA |
| | | GGCCAACGCCACCAGGAUUCCAGCAAAAACCCCGCCCGCUCCAAAG |
| | | ACACCACCCAGCUCUGGUGAACCUCCAAAAUCAGGGGAUCGCAGCG |
| | | GCUACAGCAGCCCCGGCUCCCCAGGCACUCCCGGCAGCCGCUCCCGC |
| | | ACCCCGUCCCUUCCAACCCCACCCACCCGGGAGCCCAAGAAGGUGGC |
| | | AGUGGUCCGUACUCCACCCAAGUCGCCGUCUUCCGCCAAGAGCCGC |
| | | CUGCAGACAGCCCCCGUGCCCAUGCCAGACCUGAAGAAUGUCAAGU |
| | | CCAAGAUCGGCUCCACUGAGAACCUGAAGCACCAGCCGGGAGGCGG |
| | | GAAGGUGCAGAUAAUUAAUAAGAAGCUGGAUCUUAGCAACGUCCA |
| | | GUCCAAGUGUGGCUCAAAGGAUAAUAUCAAACACGUCCCGGGAGGC |
| | | GGCAGUGUGCAAAUAGUCUACAAACCAGUUGACCUGAGCAAGGUGA |
| | | CCUCCAAGUGUGGCUCAUUAGGCAACAUCCAUCAUAAACCAGGAGG |
| | | UGGCCAGGUGGAAGUAAAAUCUGAGAAGCUUGACUUCAAGGACAG |
| | | AGUCCAGUCGAAGAUUGGGUCCCUGGACAAUAUCACCCACGUCCCU |
| | | GGCGGAGGAAAUAAAAAGAUUGAAACCCACAAGCUGACCUUCCGCG |
| | | AGAACGCCAAAGCCAAGACAGACCACGGGGCGGAGAUCGUGUACAA |
| | | GUCGCCAGUGGUGUCUGGGGACACGUCUCCACGGCAUCUCAGCAAU |
| | | GUCUCCUCCACCGGCAGCAUCGACAUGGUAGACUCGCCCCAGCUCG |
| | | CCACGCUAGCUGACGAGGUGUCUGCCUCCCUGGCCAAGCAGGGUUU |
| | | GUGAUCAGGCCCCUGGGGCGGUCAAUAAUUGUGGAGAGGAGAGAA |
| | | UGAGAGAGUGUGGAAAAAAAAAGAAUAAUGACCCGGCCCCCGCCCU |
| | | CUGCCCCCAGCUGCUCCUCGCAGUUCGGUUAAUUGGUUAAUCACUU |
| | | AACCUGCUUUUGUCACUCGGCUUUGGCUCGGGACUUCAAAAUCAGU |
| | | GAUGGGAGUAAGAGCAAAUUUCAUCUUUCCAAAUUGAUGGGUGGG |
| | | CUAGUAAUAAAAUAUUUAAAAAAAAACAUUCAAAAACAUGGCCAC |
| | | AUCCAACAUUUCCUCAGGCAAUUCCUUUUGAUUCUUUUUUCUUCCC |
| | | CCUCCAUGUAGAAGAGGGAGAAGGAGAGGCUCUGAAAGCUGCUUCU |
| | | GGGGGAUUUCAAGGGACUGGGGGUGCCAACCACCUCUGGCCCUGUU |
| | | GUGGGGGUGUCACAGAGGCAGUGGCAGCAACAAAGGAUUUGAAAC |
| | | UUGGUGUGUUCGUGGAGCCACAGGCAGACGAUGUCAACCUUGUGUG |
| | | AGUGUGACGGGGGUUGGGGUGGGGCGGGAGGCCACGGGGGAGGCC |
| | | GAGGCAGGGGCUGGGCAGAGGGGAGAGGAAGCACAAGAAGUGGGA |
| | | GUGGGAGAGGAAGCCACGUGCUGGAGAGUAGACAUCCCCCUCCUUG |
| | | CCGCUGGGAGAGCCAAGGCCUAUGCCACCUGCAGCGUCUGAGCGGC |
| | | CGCCUGUCCUUGGUGGCCGGGGGUGGGGGCCUGCUGUGGGUCAGUG |
| | | UGCCACCCUCUGCAGGGCAGCCUGUGGGAGAAGGGACAGCGGGUAA |
| | | AAAGAGAAGGCAAGCUGGCAGGAGGGUGGCACUUCGUGGAUGACCU |
| | | CCUUAGAAAAGACUGACCUUGAUGUCUUGAGAGCGCUGGCCUCUUC |
| | | CUCCCUCCCUGCAGGGUAGGGGGCCUGAGUUGAGGGGCUUCCCUCU |
| | | GCUCCACAGAAACCCUGUUUUAUUGAGUUCUGAAGGUUGGAACUGC |
| | | UGCCAUGAUUUUGGCCACUUUGCAGACCUGGGACUUUAGGGCUAAC |
| | | CAGUUCUCUUUGUAAGGACUUGUGCCUCUUGGGAGACGUCCACCCG |
| | | UUUCCAAGCCUGGGCCACUGGCAUCUCUGGAGUGUGUGGGGGUCUG |
| | | GGAGGCAGGUCCCGAGCCCCCUGUCCUUCCCACGGCCACUGCAGUC |
| | | ACCCCGUCUGCGCCGCUGUGCUGUUGUCUGCCGUGAGAGCCCAAUC |
| | | ACUGCCUAUACCCCUCAUCACACGUCACAAUGUCCCGAAUUCCCAG |
| | | CCUCACCACCCCUUCUCAGUAAUGACCCUGGUUGGUUGCAGGAGGU |
| | | ACCUACUCCAUACUGAGGGUGAAAUUAAGGGAAGGCAAAGUCCAGG |
| | | CACAAGAGUGGGACCCCAGCCUCUCACUCUCAGUUCCACUCAUCCA |
| | | ACUGGGACCCUCACCACGAAUCUCAUGAUCUGAUUCGGUUCCCUGU |
| | | CUCCUCCUCCCGUCACAGAUGUGAGCCAGGGCACUGCUCAGCUGUG |
| | | ACCCUAGGUGUUUCUGCCUUGUUGACAUGGAGAGAGCCCUUUCCCC |
| | | UGAGAAGGCCUGGCCCCUUCCUGUGCUGAGCCCACAGCAGCAGGCU |
| | | GGGUGUCUUGGUUGUCAGUGGUGGCACCAGGAUGGAAGGGCAAGG |
| | | CACCCAGGGCAGGCCCACAGUCCCGCUGUCCCCCACUUGCACCCUAG |
| | | CUUGUAGCUGCCAACCUCCCAGACAGCCCAGCCCGCUGCUCAGCUCC |
| | | ACAUGCAUAGUAUCAGCCCUCCACACCCGACAAAGGGGAACACACC |
| | | CCCUUGGAAAUGGUUCUUUUCCCCCAGUCCCAGCUGGAAGCCAUGC |
| | | UGUCUGUUCUGCUGGAGCAGCUGAACAUAUACAUAGAUGUUGCCCU |
| | | GCCCUCCCCAUCUGCACCCUGUUGAGUUGUAGUUGGAUUUGUCUGU |
| | | UUAUGCUUGGAUUCACCAGAGUGACUAUGAUAGUGAAAAGAAAAA |
| | | AAAAAAAAAAAAAGGACGCAUGUAUCUUGAAAUGCUUGUAAAGAG |
| | | GUUUCUAACCCACCCUCACGAGGUGUCUCUCACCCCCACACUGGGA |
| | | CUCGUGUGGCCUGUGUGGUGCCACCCUGCUGGGGCCUCCCAAGUUU |
| | | UGAAAGGCUUUCCUCAGCACCUGGGACCCAACAGAGACCAGCUUCU |
| | | AGCAGCUAAGGAGGCCGUUCAGCUGUGACGAAGGCCUGAAGCACAG |
| | | GAUUAGGACUGAAGCGAUGAUGUCCCCUUCCCUACUUCCCCUUGGG |
| | | GCUCCCUGUGUCAGGGCACAGACUAGGUCUUGUGGCUGGUCUGGCU |
| | | UGCGGCGCGAGGAUGGUUCUCUCUGGUCAUAGCCCGAAGUCUCAUG |
| | | GCAGUCCCAAAGGAGGCUUACAACUCCUGCAUCACAAGAAAAAGGA |
| | | AGCCACUGCCAGCUGGGGGGAUCUGCAGCUCCCAGAAGCUCCGUGA |
| | | GCCUCAGCCACCCCUCAGACUGGGUUCCUCUCCAAGCUCGCCCUCUG |
| | | GAGGGGCAGCGCAGCCUCCCACCAAGGGCCCUGCGACCACAGCAGG |
| | | GAUUGGGAUGAAUUGCCUGUCCUGGAUCUGCUCUAGAGGCCCAAGC |
| | | UGCCUGCCUGAGGAAGGAUGACUUGACAAGUCAGGAGACACUGUUC |
| | | CCAAAGCCUUGACCAGAGCACCUCAGCCCGCUGACCUUGCACAAAC |
| | | UCCAUCUGCUGCCAUGAGAAAAGGGAAGCCGCCUUUGCAAAACAUU |
| | | GCUGCCUAAAGAAACUCAGCAGCCUCAGGCCCAAUUCUGCCACUUC |
| | | UGGUUUGGGUACAGUUAAAGGCAACCCUGAGGGACUUGGCAGUAG |
| | | AAAUCCAGGGCCUCCCCUGGGGCUGGCAGCUUCGUGUGCAGCUAGA |
| | | GCUUUACCUGAAAGGAAGUCUCUGGGCCCAGAACUCUCCACCAAGA |
| | | GCCUCCCUGCCGUUCGCUGAGUCCCAGCAAUUCUCCUAAGUUGAAG |
| | | GGAUCUGAGAAGGAGAAGGAAAUGUGGGGUAGAUUUGGUGGUGGU |
| | | UAGAGAUAUGCCCCCCUCAUUACUGCCAACAGUUUCGGCUGCAUUU |
| | | CUUCACGCACCUCGGUUCCUCUUCCUGAAGUUCUUGUGCCCUGCUC |
| | | UUCAGCACCAUGGGCCUUCUUAUACGGAAGGCUCUGGGAUCUCCCC |
| | | CUUGUGGGGCAGGCUCUUGGGGCCAGCCUAAGAUCAUGGUUUAGGG |
| | | UGAUCAGUGCUGGCAGAUAAAUUGAAAAGGCACGCUGGCUUGUGA |
| | | UCUUAAAUGAGGACAAUCCCCCCAGGGCUGGGCACUCCUCCCCUCC |
| | | CCUCACUUCUCCCACCUGCAGAGCCAGUGUCCUUGGGUGGGCUAGA |
| | | UAGGAUAUACUGUAUGCCGGCUCCUUCAAGCUGCUGACUCACUUUA |
| | | UCAAUAGUUCCAUUUAAAUUGACUUCAGUGGUGAGACUGUAUCCUG |
| | | UUUGCUAUUGCUUGUUGUGCUAUGGGGGGAGGGGGGAGGAAUGUG |
| | | UAAGAUAGUUAACAUGGGCAAAGGGAGAUCUUGGGGUGCAGCACU |
| | | UAAACUGCCUCGUAACCCUUUUCAUGAUUUCAACCACAUUUGCUAG |
| | | AGGGAGGGAGCAGCCACGGAGUUAGAGGCCCUUGGGGUUUCUCUUU |
| | | UCCACUGACAGGCUUUCCCAGGCAGCUGGCUAGUUCAUUCCCUCCC |
| | | CAGCCAGGUGCAGGCGUAGGAAUAUGGACAUCUGGUUGCUUUGGCC |
| | | UGCUGCCCUCUUUCAGGGGUCCUAAGCCCACAAUCAUGCCUCCCUA |
| | | AGACCUUGGCAUCCUUCCCUCUAAGCCGUUGGCACCUCUGUGCCAC |
| | | CUCUCACACUGGCUCCAGACACACAGCCUGUGCUUUUGGAGCUGAG |
| | | AUCACUCGCUUCACCCUCCUCAUCUUUGUUCUCCAAGUAAAGCCAC |
| | | GAGGUCGGGGCGAGGGCAGAGGUGAUCACCUGCGUGUCCCAUCUAC |
| | | AGACCUGCAGCUUCAUAAAACUUCUGAUUUCUCUUCAGCUUUGAAA |
| | | AGGGUUACCCUGGGCACUGGCCUAGAGCCUCACCUCCUAAUAGACU |
| | | UAGCCCCAUGAGUUUGCCAUGUUGAGCAGGACUAUUUCUGGCACUU |
| | | GCAAGUCCCAUGAUUUCUUCGGUAAUUCUGAGGGUGGGGGGAGGG |
| | | ACAUGAAAUCAUCUUAGCUUAGCUUUCUGUCUGUGAAUGUCUAUAU |
| | | AGUGUAUUGUGUGUUUUAACAAAUGAUUUACACUGACUGUUGCUG |
| | | UAAAAGUGAAUUUGGAAAUAAAGUUAUUACUCUGAUUAAA |
| 18 | 3 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGCUGAAGAAGCAGGCAUUGGAGACACCCCCAGCCUGGAAGA |
| | | CGAAGCUGCUGGUCACGUGACCCAAGCUCGCAUGGUCAGUAAAAGC |
| | | AAAGACGGGACUGGAAGCGAUGACAAAAAAGCCAAGGGGGCUGAU |
| | | GGUAAAACGAAGAUCGCCACACCGCGGGGAGCAGCCCCUCCAGGCC |
| | | AGAAGGGCCAGGCCAACGCCACCAGGAUUCCAGCAAAAACCCCGCC |
| | | CGCUCCAAAGACACCACCCAGCUCUGGUGAACCUCCAAAAUCAGGG |
| | | GAUCGCAGCGGCUACAGCAGCCCCGGCUCCCCAGGCACUCCCGGCAG |
| | | CCGCUCCCGCACCCCGUCCCUUCCAACCCCACCCACCCGGGAGCCCA |
| | | AGAAGGUGGCAGUGGUCCGUACUCCACCCAAGUCGCCGUCUUCCGC |
| | | CAAGAGCCGCCUGCAGACAGCCCCCGUGCCCAUGCCAGACCUGAAG |
| | | AAUGUCAAGUCCAAGAUCGGCUCCACUGAGAACCUGAAGCACCAGC |
| | | CGGGAGGCGGGAAGGUGCAGAUAAUUAAUAAGAAGCUGGAUCUUA |
| | | GCAACGUCCAGUCCAAGUGUGGCUCAAAGGAUAAUAUCAAACACGU |
| | | CCCGGGAGGCGGCAGUGUGCAAAUAGUCUACAAACCAGUUGACCUG |
| | | AGCAAGGUGACCUCCAAGUGUGGCUCAUUAGGCAACAUCCAUCAUA |
| | | AACCAGGAGGUGGCCAGGUGGAAGUAAAAUCUGAGAAGCUUGACU |
| | | UCAAGGACAGAGUCCAGUCGAAGAUUGGGUCCCUGGACAAUAUCAC |
| | | CCACGUCCCUGGCGGAGGAAAUAAAAAGAUUGAAACCCACAAGCUG |
| | | ACCUUCCGCGAGAACGCCAAAGCCAAGACAGACCACGGGGCGGAGA |
| | | UCGUGUACAAGUCGCCAGUGGUGUCUGGGGACACGUCUCCACGGCA |
| | | UCUCAGCAAUGUCUCCUCCACCGGCAGCAUCGACAUGGUAGACUCG |
| | | CCCCAGCUCGCCACGCUAGCUGACGAGGUGUCUGCCUCCCUGGCCA |
| | | AGCAGGGUUUGUGAUCAGGCCCCUGGGGCGGUCAAUAAUUGUGGAG |
| | | AGGAGAGAAUGAGAGAGUGUGGAAAAAAAAAGAAUAAUGACCCGG |
| | | CCCCCGCCCUCUGCCCCCAGCUGCUCCUCGCAGUUCGGUUAAUUGGU |
| | | UAAUCACUUAACCUGCUUUUGUCACUCGGCUUUGGCUCGGGACUUC |
| | | AAAAUCAGUGAUGGGAGUAAGAGCAAAUUUCAUCUUUCCAAAUUG |
| | | AUGGGUGGGCUAGUAAUAAAAUAUUUAAAAAAAAACAUUCAAAAA |
| | | CAUGGCCACAUCCAACAUUUCCUCAGGCAAUUCCUUUUGAUUCUUU |
| | | UUUCUUCCCCCUCCAUGUAGAAGAGGGAGAAGGAGAGGCUCUGAAA |
| | | GCUGCUUCUGGGGGAUUUCAAGGGACUGGGGGUGCCAACCACCUCU |
| | | GGCCCUGUUGUGGGGGUGUCACAGAGGCAGUGGCAGCAACAAAGGA |
| | | UUUGAAACUUGGUGUGUUCGUGGAGCCACAGGCAGACGAUGUCAAC |
| | | CUUGUGUGAGUGUGACGGGGGUUGGGGUGGGGCGGGAGGCCACGG |
| | | GGGAGGCCGAGGCAGGGGCUGGGCAGAGGGGAGAGGAAGCACAAG |
| | | AAGUGGGAGUGGGAGAGGAAGCCACGUGCUGGAGAGUAGACAUCCC |
| | | CCUCCUUGCCGCUGGGAGAGCCAAGGCCUAUGCCACCUGCAGCGUC |
| | | UGAGCGGCCGCCUGUCCUUGGUGGCCGGGGGUGGGGGCCUGCUGUG |
| | | GGUCAGUGUGCCACCCUCUGCAGGGCAGCCUGUGGGAGAAGGGACA |
| | | GCGGGUAAAAAGAGAAGGCAAGCUGGCAGGAGGGUGGCACUUCGU |
| | | GGAUGACCUCCUUAGAAAAGACUGACCUUGAUGUCUUGAGAGCGCU |
| | | GGCCUCUUCCUCCCUCCCUGCAGGGUAGGGGGCCUGAGUUGAGGGG |
| | | CUUCCCUCUGCUCCACAGAAACCCUGUUUUAUUGAGUUCUGAAGGU |
| | | UGGAACUGCUGCCAUGAUUUUGGCCACUUUGCAGACCUGGGACUUU |
| | | AGGGCUAACCAGUUCUCUUUGUAAGGACUUGUGCCUCUUGGGAGAC |
| | | GUCCACCCGUUUCCAAGCCUGGGCCACUGGCAUCUCUGGAGUGUGU |
| | | GGGGGUCUGGGAGGCAGGUCCCGAGCCCCCUGUCCUUCCCACGGCC |
| | | ACUGCAGUCACCCCGUCUGCGCCGCUGUGCUGUUGUCUGCCGUGAG |
| | | AGCCCAAUCACUGCCUAUACCCCUCAUCACACGUCACAAUGUCCCG |
| | | AAUUCCCAGCCUCACCACCCCUUCUCAGUAAUGACCCUGGUUGGUU |
| | | GCAGGAGGUACCUACUCCAUACUGAGGGUGAAAUUAAGGGAAGGCA |
| | | AAGUCCAGGCACAAGAGUGGGACCCCAGCCUCUCACUCUCAGUUCC |
| | | ACUCAUCCAACUGGGACCCUCACCACGAAUCUCAUGAUCUGAUUCG |
| | | GUUCCCUGUCUCCUCCUCCCGUCACAGAUGUGAGCCAGGGCACUGC |
| | | UCAGCUGUGACCCUAGGUGUUUCUGCCUUGUUGACAUGGAGAGAGC |
| | | CCUUUCCCCUGAGAAGGCCUGGCCCCUUCCUGUGCUGAGCCCACAG |
| | | CAGCAGGCUGGGUGUCUUGGUUGUCAGUGGUGGCACCAGGAUGGAA |
| | | GGGCAAGGCACCCAGGGCAGGCCCACAGUCCCGCUGUCCCCCACUU |
| | | GCACCCUAGCUUGUAGCUGCCAACCUCCCAGACAGCCCAGCCCGCUG |
| | | CUCAGCUCCACAUGCAUAGUAUCAGCCCUCCACACCCGACAAAGGG |
| | | GAACACACCCCCUUGGAAAUGGUUCUUUUCCCCCAGUCCCAGCUGG |
| | | AAGCCAUGCUGUCUGUUCUGCUGGAGCAGCUGAACAUAUACAUAGA |
| | | UGUUGCCCUGCCCUCCCCAUCUGCACCCUGUUGAGUUGUAGUUGGA |
| | | UUUGUCUGUUUAUGCUUGGAUUCACCAGAGUGACUAUGAUAGUGA |
| | | AAAGAAAAAAAAAAAAAAAAAAGGACGCAUGUAUCUUGAAAUGCU |
| | | UGUAAAGAGGUUUCUAACCCACCCUCACGAGGUGUCUCUCACCCCC |
| | | ACACUGGGACUCGUGUGGCCUGUGUGGUGCCACCCUGCUGGGGCCU |
| | | CCCAAGUUUUGAAAGGCUUUCCUCAGCACCUGGGACCCAACAGAGA |
| | | CCAGCUUCUAGCAGCUAAGGAGGCCGUUCAGCUGUGACGAAGGCCU |
| | | GAAGCACAGGAUUAGGACUGAAGCGAUGAUGUCCCCUUCCCUACUU |
| | | CCCCUUGGGGCUCCCUGUGUCAGGGCACAGACUAGGUCUUGUGGCU |
| | | GGUCUGGCUUGCGGCGCGAGGAUGGUUCUCUCUGGUCAUAGCCCGA |
| | | AGUCUCAUGGCAGUCCCAAAGGAGGCUUACAACUCCUGCAUCACAA |
| | | GAAAAAGGAAGCCACUGCCAGCUGGGGGGAUCUGCAGCUCCCAGAA |
| | | GCUCCGUGAGCCUCAGCCACCCCUCAGACUGGGUUCCUCUCCAAGC |
| | | UCGCCCUCUGGAGGGGCAGCGCAGCCUCCCACCAAGGGCCCUGCGA |
| | | CCACAGCAGGGAUUGGGAUGAAUUGCCUGUCCUGGAUCUGCUCUAG |
| | | AGGCCCAAGCUGCCUGCCUGAGGAAGGAUGACUUGACAAGUCAGGA |
| | | GACACUGUUCCCAAAGCCUUGACCAGAGCACCUCAGCCCGCUGACC |
| | | UUGCACAAACUCCAUCUGCUGCCAUGAGAAAAGGGAAGCCGCCUUU |
| | | GCAAAACAUUGCUGCCUAAAGAAACUCAGCAGCCUCAGGCCCAAUU |
| | | CUGCCACUUCUGGUUUGGGUACAGUUAAAGGCAACCCUGAGGGACU |
| | | UGGCAGUAGAAAUCCAGGGCCUCCCCUGGGGCUGGCAGCUUCGUGU |
| | | GCAGCUAGAGCUUUACCUGAAAGGAAGUCUCUGGGCCCAGAACUCU |
| | | CCACCAAGAGCCUCCCUGCCGUUCGCUGAGUCCCAGCAAUUCUCCU |
| | | AAGUUGAAGGGAUCUGAGAAGGAGAAGGAAAUGUGGGGUAGAUUU |
| | | GGUGGUGGUUAGAGAUAUGCCCCCCUCAUUACUGCCAACAGUUUCG |
| | | GCUGCAUUUCUUCACGCACCUCGGUUCCUCUUCCUGAAGUUCUUGU |
| | | GCCCUGCUCUUCAGCACCAUGGGCCUUCUUAUACGGAAGGCUCUGG |
| | | GAUCUCCCCCUUGUGGGGCAGGCUCUUGGGGCCAGCCUAAGAUCAU |
| | | GGUUUAGGGUGAUCAGUGCUGGCAGAUAAAUUGAAAAGGCACGCU |
| | | GGCUUGUGAUCUUAAAUGAGGACAAUCCCCCCAGGGCUGGGCACUC |
| | | CUCCCCUCCCCUCACUUCUCCCACCUGCAGAGCCAGUGUCCUUGGGU |
| | | GGGCUAGAUAGGAUAUACUGUAUGCCGGCUCCUUCAAGCUGCUGAC |
| | | UCACUUUAUCAAUAGUUCCAUUUAAAUUGACUUCAGUGGUGAGACU |
| | | GUAUCCUGUUUGCUAUUGCUUGUUGUGCUAUGGGGGGAGGGGGGA |
| | | GGAAUGUGUAAGAUAGUUAACAUGGGCAAAGGGAGAUCUUGGGGU |
| | | GCAGCACUUAAACUGCCUCGUAACCCUUUUCAUGAUUUCAACCACA |
| | | UUUGCUAGAGGGAGGGAGCAGCCACGGAGUUAGAGGCCCUUGGGGU |
| | | UUCUCUUUUCCACUGACAGGCUUUCCCAGGCAGCUGGCUAGUUCAU |
| | | UCCCUCCCCAGCCAGGUGCAGGCGUAGGAAUAUGGACAUCUGGUUG |
| | | CUUUGGCCUGCUGCCCUCUUUCAGGGGUCCUAAGCCCACAAUCAUG |
| | | CCUCCCUAAGACCUUGGCAUCCUUCCCUCUAAGCCGUUGGCACCUC |
| | | UGUGCCACCUCUCACACUGGCUCCAGACACACAGCCUGUGCUUUUG |
| | | GAGCUGAGAUCACUCGCUUCACCCUCCUCAUCUUUGUUCUCCAAGU |
| | | AAAGCCACGAGGUCGGGGCGAGGGCAGAGGUGAUCACCUGCGUGUC |
| | | CCAUCUACAGACCUGCAGCUUCAUAAAACUUCUGAUUUCUCUUCAG |
| | | CUUUGAAAAGGGUUACCCUGGGCACUGGCCUAGAGCCUCACCUCCU |
| | | AAUAGACUUAGCCCCAUGAGUUUGCCAUGUUGAGCAGGACUAUUUC |
| | | UGGCACUUGCAAGUCCCAUGAUUUCUUCGGUAAUUCUGAGGGUGGG |
| | | GGGAGGGACAUGAAAUCAUCUUAGCUUAGCUUUCUGUCUGUGAAU |
| | | GUCUAUAUAGUGUAUUGUGUGUUUUAACAAAUGAUUUACACUGAC |
| | | UGUUGCUGUAAAAGUGAAUUUGGAAAUAAAGUUAUUACUCUGAUU |
| | | AAA |
| 19 | 4 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGCUGAAGAAGCAGGCAUUGGAGACACCCCCAGCCUGGAAGA |
| | | CGAAGCUGCUGGUCACGUGACCCAAGCUCGCAUGGUCAGUAAAAGC |
| | | AAAGACGGGACUGGAAGCGAUGACAAAAAAGCCAAGGGGGCUGAU |
| | | GGUAAAACGAAGAUCGCCACACCGCGGGGAGCAGCCCCUCCAGGCC |
| | | AGAAGGGCCAGGCCAACGCCACCAGGAUUCCAGCAAAAACCCCGCC |
| | | CGCUCCAAAGACACCACCCAGCUCUGGUGAACCUCCAAAAUCAGGG |
| | | GAUCGCAGCGGCUACAGCAGCCCCGGCUCCCCAGGCACUCCCGGCAG |
| | | CCGCUCCCGCACCCCGUCCCUUCCAACCCCACCCACCCGGGAGCCCA |
| | | AGAAGGUGGCAGUGGUCCGUACUCCACCCAAGUCGCCGUCUUCCGC |
| | | CAAGAGCCGCCUGCAGACAGCCCCCGUGCCCAUGCCAGACCUGAAG |
| | | AAUGUCAAGUCCAAGAUCGGCUCCACUGAGAACCUGAAGCACCAGC |
| | | CGGGAGGCGGGAAGGUGCAAAUAGUCUACAAACCAGUUGACCUGAG |
| | | CAAGGUGACCUCCAAGUGUGGCUCAUUAGGCAACAUCCAUCAUAAA |
| | | CCAGGAGGUGGCCAGGUGGAAGUAAAAUCUGAGAAGCUUGACUUCA |
| | | AGGACAGAGUCCAGUCGAAGAUUGGGUCCCUGGACAAUAUCACCCA |
| | | CGUCCCUGGCGGAGGAAAUAAAAAGAUUGAAACCCACAAGCUGACC |
| | | UUCCGCGAGAACGCCAAAGCCAAGACAGACCACGGGGCGGAGAUCG |
| | | UGUACAAGUCGCCAGUGGUGUCUGGGGACACGUCUCCACGGCAUCU |
| | | CAGCAAUGUCUCCUCCACCGGCAGCAUCGACAUGGUAGACUCGCCC |
| | | CAGCUCGCCACGCUAGCUGACGAGGUGUCUGCCUCCCUGGCCAAGC |
| | | AGGGUUUGUGAUCAGGCCCCUGGGGCGGUCAAUAAUUGUGGAGAG |
| | | GAGAGAAUGAGAGAGUGUGGAAAAAAAAAGAAUAAUGACCCGGCC |
| | | CCCGCCCUCUGCCCCCAGCUGCUCCUCGCAGUUCGGUUAAUUGGUU |
| | | AAUCACUUAACCUGCUUUUGUCACUCGGCUUUGGCUCGGGACUUCA |
| | | AAAUCAGUGAUGGGAGUAAGAGCAAAUUUCAUCUUUCCAAAUUGA |
| | | UGGGUGGGCUAGUAAUAAAAUAUUUAAAAAAAAACAUUCAAAAAC |
| | | AUGGCCACAUCCAACAUUUCCUCAGGCAAUUCCUUUUGAUUCUUUU |
| | | UUCUUCCCCCUCCAUGUAGAAGAGGGAGAAGGAGAGGCUCUGAAAG |
| | | CUGCUUCUGGGGGAUUUCAAGGGACUGGGGGUGCCAACCACCUCUG |
| | | GCCCUGUUGUGGGGGUGUCACAGAGGCAGUGGCAGCAACAAAGGAU |
| | | UUGAAACUUGGUGUGUUCGUGGAGCCACAGGCAGACGAUGUCAACC |
| | | UUGUGUGAGUGUGACGGGGGUUGGGGUGGGGCGGGAGGCCACGGG |
| | | GGAGGCCGAGGCAGGGGCUGGGCAGAGGGGAGAGGAAGCACAAGA |
| | | AGUGGGAGUGGGAGAGGAAGCCACGUGCUGGAGAGUAGACAUCCCC |
| | | CUCCUUGCCGCUGGGAGAGCCAAGGCCUAUGCCACCUGCAGCGUCU |
| | | GAGCGGCCGCCUGUCCUUGGUGGCCGGGGGUGGGGGCCUGCUGUGG |
| | | GUCAGUGUGCCACCCUCUGCAGGGCAGCCUGUGGGAGAAGGGACAG |
| | | CGGGUAAAAAGAGAAGGCAAGCUGGCAGGAGGGUGGCACUUCGUG |
| | | GAUGACCUCCUUAGAAAAGACUGACCUUGAUGUCUUGAGAGCGCUG |
| | | GCCUCUUCCUCCCUCCCUGCAGGGUAGGGGGCCUGAGUUGAGGGGC |
| | | UUCCCUCUGCUCCACAGAAACCCUGUUUUAUUGAGUUCUGAAGGUU |
| | | GGAACUGCUGCCAUGAUUUUGGCCACUUUGCAGACCUGGGACUUUA |
| | | GGGCUAACCAGUUCUCUUUGUAAGGACUUGUGCCUCUUGGGAGACG |
| | | UCCACCCGUUUCCAAGCCUGGGCCACUGGCAUCUCUGGAGUGUGUG |
| | | GGGGUCUGGGAGGCAGGUCCCGAGCCCCCUGUCCUUCCCACGGCCA |
| | | CUGCAGUCACCCCGUCUGCGCCGCUGUGCUGUUGUCUGCCGUGAGA |
| | | GCCCAAUCACUGCCUAUACCCCUCAUCACACGUCACAAUGUCCCGA |
| | | AUUCCCAGCCUCACCACCCCUUCUCAGUAAUGACCCUGGUUGGUUG |
| | | CAGGAGGUACCUACUCCAUACUGAGGGUGAAAUUAAGGGAAGGCAA |
| | | AGUCCAGGCACAAGAGUGGGACCCCAGCCUCUCACUCUCAGUUCCA |
| | | CUCAUCCAACUGGGACCCUCACCACGAAUCUCAUGAUCUGAUUCGG |
| | | UUCCCUGUCUCCUCCUCCCGUCACAGAUGUGAGCCAGGGCACUGCU |
| | | CAGCUGUGACCCUAGGUGUUUCUGCCUUGUUGACAUGGAGAGAGCC |
| | | CUUUCCCCUGAGAAGGCCUGGCCCCUUCCUGUGCUGAGCCCACAGC |
| | | AGCAGGCUGGGUGUCUUGGUUGUCAGUGGUGGCACCAGGAUGGAA |
| | | GGGCAAGGCACCCAGGGCAGGCCCACAGUCCCGCUGUCCCCCACUU |
| | | GCACCCUAGCUUGUAGCUGCCAACCUCCCAGACAGCCCAGCCCGCUG |
| | | CUCAGCUCCACAUGCAUAGUAUCAGCCCUCCACACCCGACAAAGGG |
| | | GAACACACCCCCUUGGAAAUGGUUCUUUUCCCCCAGUCCCAGCUGG |
| | | AAGCCAUGCUGUCUGUUCUGCUGGAGCAGCUGAACAUAUACAUAGA |
| | | UGUUGCCCUGCCCUCCCCAUCUGCACCCUGUUGAGUUGUAGUUGGA |
| | | UUUGUCUGUUUAUGCUUGGAUUCACCAGAGUGACUAUGAUAGUGA |
| | | AAAGAAAAAAAAAAAAAAAAAAGGACGCAUGUAUCUUGAAAUGCU |
| | | UGUAAAGAGGUUUCUAACCCACCCUCACGAGGUGUCUCUCACCCCC |
| | | ACACUGGGACUCGUGUGGCCUGUGUGGUGCCACCCUGCUGGGGCCU |
| | | CCCAAGUUUUGAAAGGCUUUCCUCAGCACCUGGGACCCAACAGAGA |
| | | CCAGCUUCUAGCAGCUAAGGAGGCCGUUCAGCUGUGACGAAGGCCU |
| | | GAAGCACAGGAUUAGGACUGAAGCGAUGAUGUCCCCUUCCCUACUU |
| | | CCCCUUGGGGCUCCCUGUGUCAGGGCACAGACUAGGUCUUGUGGCU |
| | | GGUCUGGCUUGCGGCGCGAGGAUGGUUCUCUCUGGUCAUAGCCCGA |
| | | AGUCUCAUGGCAGUCCCAAAGGAGGCUUACAACUCCUGCAUCACAA |
| | | GAAAAAGGAAGCCACUGCCAGCUGGGGGGAUCUGCAGCUCCCAGAA |
| | | GCUCCGUGAGCCUCAGCCACCCCUCAGACUGGGUUCCUCUCCAAGC |
| | | UCGCCCUCUGGAGGGGCAGCGCAGCCUCCCACCAAGGGCCCUGCGA |
| | | CCACAGCAGGGAUUGGGAUGAAUUGCCUGUCCUGGAUCUGCUCUAG |
| | | AGGCCCAAGCUGCCUGCCUGAGGAAGGAUGACUUGACAAGUCAGGA |
| | | GACACUGUUCCCAAAGCCUUGACCAGAGCACCUCAGCCCGCUGACC |
| | | UUGCACAAACUCCAUCUGCUGCCAUGAGAAAAGGGAAGCCGCCUUU |
| | | GCAAAACAUUGCUGCCUAAAGAAACUCAGCAGCCUCAGGCCCAAUU |
| | | CUGCCACUUCUGGUUUGGGUACAGUUAAAGGCAACCCUGAGGGACU |
| | | UGGCAGUAGAAAUCCAGGGCCUCCCCUGGGGCUGGCAGCUUCGUGU |
| | | GCAGCUAGAGCUUUACCUGAAAGGAAGUCUCUGGGCCCAGAACUCU |
| | | CCACCAAGAGCCUCCCUGCCGUUCGCUGAGUCCCAGCAAUUCUCCU |
| | | AAGUUGAAGGGAUCUGAGAAGGAGAAGGAAAUGUGGGGUAGAUUU |
| | | GGUGGUGGUUAGAGAUAUGCCCCCCUCAUUACUGCCAACAGUUUCG |
| | | GCUGCAUUUCUUCACGCACCUCGGUUCCUCUUCCUGAAGUUCUUGU |
| | | GCCCUGCUCUUCAGCACCAUGGGCCUUCUUAUACGGAAGGCUCUGG |
| | | GAUCUCCCCCUUGUGGGGCAGGCUCUUGGGGCCAGCCUAAGAUCAU |
| | | GGUUUAGGGUGAUCAGUGCUGGCAGAUAAAUUGAAAAGGCACGCU |
| | | GGCUUGUGAUCUUAAAUGAGGACAAUCCCCCCAGGGCUGGGCACUC |
| | | CUCCCCUCCCCUCACUUCUCCCACCUGCAGAGCCAGUGUCCUUGGGU |
| | | GGGCUAGAUAGGAUAUACUGUAUGCCGGCUCCUUCAAGCUGCUGAC |
| | | UCACUUUAUCAAUAGUUCCAUUUAAAUUGACUUCAGUGGUGAGACU |
| | | GUAUCCUGUUUGCUAUUGCUUGUUGUGCUAUGGGGGGAGGGGGGA |
| | | GGAAUGUGUAAGAUAGUUAACAUGGGCAAAGGGAGAUCUUGGGGU |
| | | GCAGCACUUAAACUGCCUCGUAACCCUUUUCAUGAUUUCAACCACA |
| | | UUUGCUAGAGGGAGGGAGCAGCCACGGAGUUAGAGGCCCUUGGGGU |
| | | UUCUCUUUUCCACUGACAGGCUUUCCCAGGCAGCUGGCUAGUUCAU |
| | | UCCCUCCCCAGCCAGGUGCAGGCGUAGGAAUAUGGACAUCUGGUUG |
| | | CUUUGGCCUGCUGCCCUCUUUCAGGGGUCCUAAGCCCACAAUCAUG |
| | | CCUCCCUAAGACCUUGGCAUCCUUCCCUCUAAGCCGUUGGCACCUC |
| | | UGUGCCACCUCUCACACUGGCUCCAGACACACAGCCUGUGCUUUUG |
| | | GAGCUGAGAUCACUCGCUUCACCCUCCUCAUCUUUGUUCUCCAAGU |
| | | AAAGCCACGAGGUCGGGGCGAGGGCAGAGGUGAUCACCUGCGUGUC |
| | | CCAUCUACAGACCUGCAGCUUCAUAAAACUUCUGAUUUCUCUUCAG |
| | | CUUUGAAAAGGGUUACCCUGGGCACUGGCCUAGAGCCUCACCUCCU |
| | | AAUAGACUUAGCCCCAUGAGUUUGCCAUGUUGAGCAGGACUAUUUC |
| | | UGGCACUUGCAAGUCCCAUGAUUUCUUCGGUAAUUCUGAGGGUGGG |
| | | GGGAGGGACAUGAAAUCAUCUUAGCUUAGCUUUCUGUCUGUGAAU |
| | | GUCUAUAUAGUGUAUUGUGUGUUUUAACAAAUGAUUUACACUGAC |
| | | UGUUGCUGUAAAAGUGAAUUUGGAAAUAAAGUUAUUACUCUGAUU |
| | | AAA |
| 20 | 5 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GCUGAAGAAGCAGGCAUUGGAGACACCCCCAGCCUGGAAGACGAAG |
| | | CUGCUGGUCACGUGACCCAAGCUCGCAUGGUCAGUAAAAGCAAAGA |
| | | CGGGACUGGAAGCGAUGACAAAAAAGCCAAGGGGGCUGAUGGUAA |
| | | AACGAAGAUCGCCACACCGCGGGGAGCAGCCCCUCCAGGCCAGAAG |
| | | GGCCAGGCCAACGCCACCAGGAUUCCAGCAAAAACCCCGCCCGCUCC |
| | | AAAGACACCACCCAGCUCUGGUGAACCUCCAAAAUCAGGGGAUCGC |
| | | AGCGGCUACAGCAGCCCCGGCUCCCCAGGCACUCCCGGCAGCCGCUC |
| | | CCGCACCCCGUCCCUUCCAACCCCACCCACCCGGGAGCCCAAGAAGG |
| | | UGGCAGUGGUCCGUACUCCACCCAAGUCGCCGUCUUCCGCCAAGAG |
| | | CCGCCUGCAGACAGCCCCCGUGCCCAUGCCAGACCUGAAGAAUGUC |
| | | AAGUCCAAGAUCGGCUCCACUGAGAACCUGAAGCACCAGCCGGGAG |
| | | GCGGGAAGGUGCAGAUAAUUAAUAAGAAGCUGGAUCUUAGCAACG |
| | | UCCAGUCCAAGUGUGGCUCAAAGGAUAAUAUCAAACACGUCCCGGG |
| | | AGGCGGCAGUGUGCAAAUAGUCUACAAACCAGUUGACCUGAGCAAG |
| | | GUGACCUCCAAGUGUGGCUCAUUAGGCAACAUCCAUCAUAAACCAG |
| | | GAGGUGGCCAGGUGGAAGUAAAAUCUGAGAAGCUUGACUUCAAGG |
| | | ACAGAGUCCAGUCGAAGAUUGGGUCCCUGGACAAUAUCACCCACGU |
| | | CCCUGGCGGAGGAAAUAAAAAGAUUGAAACCCACAAGCUGACCUUC |
| | | CGCGAGAACGCCAAAGCCAAGACAGACCACGGGGCGGAGAUCGUGU |
| | | ACAAGUCGCCAGUGGUGUCUGGGGACACGUCUCCACGGCAUCUCAG |
| | | CAAUGUCUCCUCCACCGGCAGCAUCGACAUGGUAGACUCGCCCCAG |
| | | CUCGCCACGCUAGCUGACGAGGUGUCUGCCUCCCUGGCCAAGCAGG |
| | | GUUUGUGAUCAGGCCCCUGGGGCGGUCAAUAAUUGUGGAGAGGAG |
| | | AGAAUGAGAGAGUGUGGAAAAAAAAAGAAUAAUGACCCGGCCCCCG |
| | | CCCUCUGCCCCCAGCUGCUCCUCGCAGUUCGGUUAAUUGGUUAAUC |
| | | ACUUAACCUGCUUUUGUCACUCGGCUUUGGCUCGGGACUUCAAAAU |
| | | CAGUGAUGGGAGUAAGAGCAAAUUUCAUCUUUCCAAAUUGAUGGG |
| | | UGGGCUAGUAAUAAAAUAUUUAAAAAAAAACAUUCAAAAACAUGG |
| | | CCACAUCCAACAUUUCCUCAGGCAAUUCCUUUUGAUUCUUUUUUCU |
| | | UCCCCCUCCAUGUAGAAGAGGGAGAAGGAGAGGCUCUGAAAGCUGC |
| | | UUCUGGGGGAUUUCAAGGGACUGGGGGUGCCAACCACCUCUGGCCC |
| | | UGUUGUGGGGGUGUCACAGAGGCAGUGGCAGCAACAAAGGAUUUG |
| | | AAACUUGGUGUGUUCGUGGAGCCACAGGCAGACGAUGUCAACCUUG |
| | | UGUGAGUGUGACGGGGGUUGGGGUGGGGCGGGAGGCCACGGGGGA |
| | | GGCCGAGGCAGGGGCUGGGCAGAGGGGAGAGGAAGCACAAGAAGU |
| | | GGGAGUGGGAGAGGAAGCCACGUGCUGGAGAGUAGACAUCCCCCUC |
| | | CUUGCCGCUGGGAGAGCCAAGGCCUAUGCCACCUGCAGCGUCUGAG |
| | | CGGCCGCCUGUCCUUGGUGGCCGGGGGUGGGGGCCUGCUGUGGGUC |
| | | AGUGUGCCACCCUCUGCAGGGCAGCCUGUGGGAGAAGGGACAGCGG |
| | | GUAAAAAGAGAAGGCAAGCUGGCAGGAGGGUGGCACUUCGUGGAU |
| | | GACCUCCUUAGAAAAGACUGACCUUGAUGUCUUGAGAGCGCUGGCC |
| | | UCUUCCUCCCUCCCUGCAGGGUAGGGGGCCUGAGUUGAGGGGCUUC |
| | | CCUCUGCUCCACAGAAACCCUGUUUUAUUGAGUUCUGAAGGUUGGA |
| | | ACUGCUGCCAUGAUUUUGGCCACUUUGCAGACCUGGGACUUUAGGG |
| | | CUAACCAGUUCUCUUUGUAAGGACUUGUGCCUCUUGGGAGACGUCC |
| | | ACCCGUUUCCAAGCCUGGGCCACUGGCAUCUCUGGAGUGUGUGGGG |
| | | GUCUGGGAGGCAGGUCCCGAGCCCCCUGUCCUUCCCACGGCCACUG |
| | | CAGUCACCCCGUCUGCGCCGCUGUGCUGUUGUCUGCCGUGAGAGCC |
| | | CAAUCACUGCCUAUACCCCUCAUCACACGUCACAAUGUCCCGAAUU |
| | | CCCAGCCUCACCACCCCUUCUCAGUAAUGACCCUGGUUGGUUGCAG |
| | | GAGGUACCUACUCCAUACUGAGGGUGAAAUUAAGGGAAGGCAAAG |
| | | UCCAGGCACAAGAGUGGGACCCCAGCCUCUCACUCUCAGUUCCACU |
| | | CAUCCAACUGGGACCCUCACCACGAAUCUCAUGAUCUGAUUCGGUU |
| | | CCCUGUCUCCUCCUCCCGUCACAGAUGUGAGCCAGGGCACUGCUCA |
| | | GCUGUGACCCUAGGUGUUUCUGCCUUGUUGACAUGGAGAGAGCCCU |
| | | UUCCCCUGAGAAGGCCUGGCCCCUUCCUGUGCUGAGCCCACAGCAG |
| | | CAGGCUGGGUGUCUUGGUUGUCAGUGGUGGCACCAGGAUGGAAGG |
| | | GCAAGGCACCCAGGGCAGGCCCACAGUCCCGCUGUCCCCCACUUGCA |
| | | CCCUAGCUUGUAGCUGCCAACCUCCCAGACAGCCCAGCCCGCUGCUC |
| | | AGCUCCACAUGCAUAGUAUCAGCCCUCCACACCCGACAAAGGGGAA |
| | | CACACCCCCUUGGAAAUGGUUCUUUUCCCCCAGUCCCAGCUGGAAG |
| | | CCAUGCUGUCUGUUCUGCUGGAGCAGCUGAACAUAUACAUAGAUGU |
| | | UGCCCUGCCCUCCCCAUCUGCACCCUGUUGAGUUGUAGUUGGAUUU |
| | | GUCUGUUUAUGCUUGGAUUCACCAGAGUGACUAUGAUAGUGAAAA |
| | | GAAAAAAAAAAAAAAAAAAGGACGCAUGUAUCUUGAAAUGCUUGU |
| | | AAAGAGGUUUCUAACCCACCCUCACGAGGUGUCUCUCACCCCCACA |
| | | CUGGGACUCGUGUGGCCUGUGUGGUGCCACCCUGCUGGGGCCUCCC |
| | | AAGUUUUGAAAGGCUUUCCUCAGCACCUGGGACCCAACAGAGACCA |
| | | GCUUCUAGCAGCUAAGGAGGCCGUUCAGCUGUGACGAAGGCCUGAA |
| | | GCACAGGAUUAGGACUGAAGCGAUGAUGUCCCCUUCCCUACUUCCC |
| | | CUUGGGGCUCCCUGUGUCAGGGCACAGACUAGGUCUUGUGGCUGGU |
| | | CUGGCUUGCGGCGCGAGGAUGGUUCUCUCUGGUCAUAGCCCGAAGU |
| | | CUCAUGGCAGUCCCAAAGGAGGCUUACAACUCCUGCAUCACAAGAA |
| | | AAAGGAAGCCACUGCCAGCUGGGGGGAUCUGCAGCUCCCAGAAGCU |
| | | CCGUGAGCCUCAGCCACCCCUCAGACUGGGUUCCUCUCCAAGCUCGC |
| | | CCUCUGGAGGGGCAGCGCAGCCUCCCACCAAGGGCCCUGCGACCAC |
| | | AGCAGGGAUUGGGAUGAAUUGCCUGUCCUGGAUCUGCUCUAGAGGC |
| | | CCAAGCUGCCUGCCUGAGGAAGGAUGACUUGACAAGUCAGGAGACA |
| | | CUGUUCCCAAAGCCUUGACCAGAGCACCUCAGCCCGCUGACCUUGC |
| | | ACAAACUCCAUCUGCUGCCAUGAGAAAAGGGAAGCCGCCUUUGCAA |
| | | AACAUUGCUGCCUAAAGAAACUCAGCAGCCUCAGGCCCAAUUCUGC |
| | | CACUUCUGGUUUGGGUACAGUUAAAGGCAACCCUGAGGGACUUGGC |
| | | AGUAGAAAUCCAGGGCCUCCCCUGGGGCUGGCAGCUUCGUGUGCAG |
| | | CUAGAGCUUUACCUGAAAGGAAGUCUCUGGGCCCAGAACUCUCCAC |
| | | CAAGAGCCUCCCUGCCGUUCGCUGAGUCCCAGCAAUUCUCCUAAGU |
| | | UGAAGGGAUCUGAGAAGGAGAAGGAAAUGUGGGGUAGAUUUGGUG |
| | | GUGGUUAGAGAUAUGCCCCCCUCAUUACUGCCAACAGUUUCGGCUG |
| | | CAUUUCUUCACGCACCUCGGUUCCUCUUCCUGAAGUUCUUGUGCCC |
| | | UGCUCUUCAGCACCAUGGGCCUUCUUAUACGGAAGGCUCUGGGAUC |
| | | UCCCCCUUGUGGGGCAGGCUCUUGGGGCCAGCCUAAGAUCAUGGUU |
| | | UAGGGUGAUCAGUGCUGGCAGAUAAAUUGAAAAGGCACGCUGGCU |
| | | UGUGAUCUUAAAUGAGGACAAUCCCCCCAGGGCUGGGCACUCCUCC |
| | | CCUCCCCUCACUUCUCCCACCUGCAGAGCCAGUGUCCUUGGGUGGG |
| | | CUAGAUAGGAUAUACUGUAUGCCGGCUCCUUCAAGCUGCUGACUCA |
| | | CUUUAUCAAUAGUUCCAUUUAAAUUGACUUCAGUGGUGAGACUGU |
| | | AUCCUGUUUGCUAUUGCUUGUUGUGCUAUGGGGGGAGGGGGGAGG |
| | | AAUGUGUAAGAUAGUUAACAUGGGCAAAGGGAGAUCUUGGGGUGC |
| | | AGCACUUAAACUGCCUCGUAACCCUUUUCAUGAUUUCAACCACAUU |
| | | UGCUAGAGGGAGGGAGCAGCCACGGAGUUAGAGGCCCUUGGGGUUU |
| | | CUCUUUUCCACUGACAGGCUUUCCCAGGCAGCUGGCUAGUUCAUUC |
| | | CCUCCCCAGCCAGGUGCAGGCGUAGGAAUAUGGACAUCUGGUUGCU |
| | | UUGGCCUGCUGCCCUCUUUCAGGGGUCCUAAGCCCACAAUCAUGCC |
| | | UCCCUAAGACCUUGGCAUCCUUCCCUCUAAGCCGUUGGCACCUCUG |
| | | UGCCACCUCUCACACUGGCUCCAGACACACAGCCUGUGCUUUUGGA |
| | | GCUGAGAUCACUCGCUUCACCCUCCUCAUCUUUGUUCUCCAAGUAA |
| | | AGCCACGAGGUCGGGGCGAGGGCAGAGGUGAUCACCUGCGUGUCCC |
| | | AUCUACAGACCUGCAGCUUCAUAAAACUUCUGAUUUCUCUUCAGCU |
| | | UUGAAAAGGGUUACCCUGGGCACUGGCCUAGAGCCUCACCUCCUAA |
| | | UAGACUUAGCCCCAUGAGUUUGCCAUGUUGAGCAGGACUAUUUCUG |
| | | GCACUUGCAAGUCCCAUGAUUUCUUCGGUAAUUCUGAGGGUGGGGG |
| | | GAGGGACAUGAAAUCAUCUUAGCUUAGCUUUCUGUCUGUGAAUGUC |
| | | UAUAUAGUGUAUUGUGUGUUUUAACAAAUGAUUUACACUGACUGU |
| | | UGCUGUAAAAGUGAAUUUGGAAAUAAAGUUAUUACUCUGAUUAAA |
| 21 | 6 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GAUGUGACAGCACCCUUAGUGGAUGAGGGAGCUCCCGGCAAGCAGG |
| | | CUGCCGCGCAGCCCCACACGGAGAUCCCAGAAGGAACCACAGCUGA |
| | | AGAAGCAGGCAUUGGAGACACCCCCAGCCUGGAAGACGAAGCUGCU |
| | | GGUCACGUGACCCAAGAGCCUGAAAGUGGUAAGGUGGUCCAGGAAG |
| | | GCUUCCUCCGAGAGCCAGGCCCCCCAGGUCUGAGCCACCAGCUCAU |
| | | GUCCGGCAUGCCUGGGGCUCCCCUCCUGCCUGAGGGCCCCAGAGAG |
| | | GCCACACGCCAACCUUCGGGGACAGGACCUGAGGACACAGAGGGCG |
| | | GCCGCCACGCCCCUGAGCUGCUCAAGCACCAGCUUCUAGGAGACCU |
| | | GCACCAGGAGGGGCCGCCGCUGAAGGGGGCAGGGGGCAAAGAGAGG |
| | | CCGGGGAGCAAGGAGGAGGUGGAUGAAGACCGCGACGUCGAUGAGU |
| | | CCUCCCCCCAAGACUCCCCUCCCUCCAAGGCCUCCCCAGCCCAAGAU |
| | | GGGCGGCCUCCCCAGACAGCCGCCAGAGAAGCCACCAGCAUCCCAG |
| | | GCUUCCCAGCGGAGGGUGCCAUCCCCCUCCCUGUGGAUUUCCUCUC |
| | | CAAAGUUUCCACAGAGAUCCCAGCCUCAGAGCCCGACGGGCCCAGU |
| | | GUAGGGCGGGCCAAAGGGCAGGAUGCCCCCCUGGAGUUCACGUUUC |
| | | ACGUGGAAAUCACACCCAACGUGCAGAAGGAGCAGGCGCACUCGGA |
| | | GGAGCAUUUGGGAAGGGCUGCAUUUCCAGGGGCCCCUGGAGAGGGG |
| | | CCAGAGGCCCGGGGCCCCUCUUUGGGAGAGGACACAAAAGAGGCUG |
| | | ACCUUCCAGAGCCCUCUGAAAAGCAGCCUGCUGCUGCUCCGCGGGG |
| | | GAAGCCCGUCAGCCGGGUCCCUCAACUCAAAGCUCGCAUGGUCAGU |
| | | AAAAGCAAAGACGGGACUGGAAGCGAUGACAAAAAAGCCAAGACAU |
| | | CCACACGUUCCUCUGCUAAAACCUUGAAAAAUAGGCCUUGCCUUAG |
| | | CCCCAAACACCCCACUCCUGGUAGCUCAGACCCUCUGAUCCAACCCU |
| | | CCAGCCCUGCUGUGUGCCCAGAGCCACCUUCCUCUCCUAAAUACGU |
| | | CUCUUCUGUCACUUCCCGAACUGGCAGUUCUGGAGCAAAGGAGAUG |
| | | AAACUCAAGGGGGCUGAUGGUAAAACGAAGAUCGCCACACCGCGGG |
| | | GAGCAGCCCCUCCAGGCCAGAAGGGCCAGGCCAACGCCACCAGGAU |
| | | UCCAGCAAAAACCCCGCCCGCUCCAAAGACACCACCCAGCUCUGCGA |
| | | CUAAGCAAGUCCAGAGAAGACCACCCCCUGCAGGGCCCAGAUCUGA |
| | | GAGAGGUGAACCUCCAAAAUCAGGGGAUCGCAGCGGCUACAGCAGC |
| | | CCCGGCUCCCCAGGCACUCCCGGCAGCCGCUCCCGCACCCCGUCCCU |
| | | UCCAACCCCACCCACCCGGGAGCCCAAGAAGGUGGCAGUGGUCCGU |
| | | ACUCCACCCAAGUCGCCGUCUUCCGCCAAGAGCCGCCUGCAGACAGC |
| | | CCCCGUGCCCAUGCCAGACCUGAAGAAUGUCAAGUCCAAGAUCGGC |
| | | UCCACUGAGAACCUGAAGCACCAGCCGGGAGGCGGGAAGGUGCAGA |
| | | UAAUUAAUAAGAAGCUGGAUCUUAGCAACGUCCAGUCCAAGUGUGG |
| | | CUCAAAGGAUAAUAUCAAACACGUCCCGGGAGGCGGCAGUGUGCAA |
| | | AUAGUCUACAAACCAGUUGACCUGAGCAAGGUGACCUCCAAGUGUG |
| | | GCUCAUUAGGCAACAUCCAUCAUAAACCAGGAGGUGGCCAGGUGGA |
| | | AGUAAAAUCUGAGAAGCUUGACUUCAAGGACAGAGUCCAGUCGAAG |
| | | AUUGGGUCCCUGGACAAUAUCACCCACGUCCCUGGCGGAGGAAAUA |
| | | AAAAGAUUGAAACCCACAAGCUGACCUUCCGCGAGAACGCCAAAGC |
| | | CAAGACAGACCACGGGGCGGAGAUCGUGUACAAGUCGCCAGUGGUG |
| | | UCUGGGGACACGUCUCCACGGCAUCUCAGCAAUGUCUCCUCCACCG |
| | | GCAGCAUCGACAUGGUAGACUCGCCCCAGCUCGCCACGCUAGCUGA |
| | | CGAGGUGUCUGCCUCCCUGGCCAAGCAGGGUUUGUGAUCAGGCCCC |
| | | UGGGGCGGUCAAUAAUUGUGGAGAGGAGAGAAUGAGAGAGUGUGG |
| | | AAAAAAAAAGAAUAAUGACCCGGCCCCCGCCCUCUGCCCCCAGCUG |
| | | CUCCUCGCAGUUCGGUUAAUUGGUUAAUCACUUAACCUGCUUUUGU |
| | | CACUCGGCUUUGGCUCGGGACUUCAAAAUCAGUGAUGGGAGUAAGA |
| | | GCAAAUUUCAUCUUUCCAAAUUGAUGGGUGGGCUAGUAAUAAAAU |
| | | AUUUAAAAAAAAACAUUCAAAAACAUGGCCACAUCCAACAUUUCCU |
| | | CAGGCAAUUCCUUUUGAUUCUUUUUUCUUCCCCCUCCAUGUAGAAG |
| | | AGGGAGAAGGAGAGGCUCUGAAAGCUGCUUCUGGGGGAUUUCAAG |
| | | GGACUGGGGGUGCCAACCACCUCUGGCCCUGUUGUGGGGGUGUCAC |
| | | AGAGGCAGUGGCAGCAACAAAGGAUUUGAAACUUGGUGUGUUCGU |
| | | GGAGCCACAGGCAGACGAUGUCAACCUUGUGUGAGUGUGACGGGGG |
| | | UUGGGGUGGGGCGGGAGGCCACGGGGGAGGCCGAGGCAGGGGCUGG |
| | | GCAGAGGGGAGAGGAAGCACAAGAAGUGGGAGUGGGAGAGGAAGC |
| | | CACGUGCUGGAGAGUAGACAUCCCCCUCCUUGCCGCUGGGAGAGCC |
| | | AAGGCCUAUGCCACCUGCAGCGUCUGAGCGGCCGCCUGUCCUUGGU |
| | | GGCCGGGGGUGGGGGCCUGCUGUGGGUCAGUGUGCCACCCUCUGCA |
| | | GGGCAGCCUGUGGGAGAAGGGACAGCGGGUAAAAAGAGAAGGCAA |
| | | GCUGGCAGGAGGGUGGCACUUCGUGGAUGACCUCCUUAGAAAAGAC |
| | | UGACCUUGAUGUCUUGAGAGCGCUGGCCUCUUCCUCCCUCCCUGCA |
| | | GGGUAGGGGGCCUGAGUUGAGGGGCUUCCCUCUGCUCCACAGAAAC |
| | | CCUGUUUUAUUGAGUUCUGAAGGUUGGAACUGCUGCCAUGAUUUU |
| | | GGCCACUUUGCAGACCUGGGACUUUAGGGCUAACCAGUUCUCUUUG |
| | | UAAGGACUUGUGCCUCUUGGGAGACGUCCACCCGUUUCCAAGCCUG |
| | | GGCCACUGGCAUCUCUGGAGUGUGUGGGGGUCUGGGAGGCAGGUCC |
| | | CGAGCCCCCUGUCCUUCCCACGGCCACUGCAGUCACCCCGUCUGCGC |
| | | CGCUGUGCUGUUGUCUGCCGUGAGAGCCCAAUCACUGCCUAUACCC |
| | | CUCAUCACACGUCACAAUGUCCCGAAUUCCCAGCCUCACCACCCCUU |
| | | CUCAGUAAUGACCCUGGUUGGUUGCAGGAGGUACCUACUCCAUACU |
| | | GAGGGUGAAAUUAAGGGAAGGCAAAGUCCAGGCACAAGAGUGGGA |
| | | CCCCAGCCUCUCACUCUCAGUUCCACUCAUCCAACUGGGACCCUCAC |
| | | CACGAAUCUCAUGAUCUGAUUCGGUUCCCUGUCUCCUCCUCCCGUC |
| | | ACAGAUGUGAGCCAGGGCACUGCUCAGCUGUGACCCUAGGUGUUUC |
| | | UGCCUUGUUGACAUGGAGAGAGCCCUUUCCCCUGAGAAGGCCUGGC |
| | | CCCUUCCUGUGCUGAGCCCACAGCAGCAGGCUGGGUGUCUUGGUUG |
| | | UCAGUGGUGGCACCAGGAUGGAAGGGCAAGGCACCCAGGGCAGGCC |
| | | CACAGUCCCGCUGUCCCCCACUUGCACCCUAGCUUGUAGCUGCCAAC |
| | | CUCCCAGACAGCCCAGCCCGCUGCUCAGCUCCACAUGCAUAGUAUC |
| | | AGCCCUCCACACCCGACAAAGGGGAACACACCCCCUUGGAAAUGGU |
| | | UCUUUUCCCCCAGUCCCAGCUGGAAGCCAUGCUGUCUGUUCUGCUG |
| | | GAGCAGCUGAACAUAUACAUAGAUGUUGCCCUGCCCUCCCCAUCUG |
| | | CACCCUGUUGAGUUGUAGUUGGAUUUGUCUGUUUAUGCUUGGAUU |
| | | CACCAGAGUGACUAUGAUAGUGAAAAGAAAAAAAAAAAAAAAAAA |
| | | GGACGCAUGUAUCUUGAAAUGCUUGUAAAGAGGUUUCUAACCCACC |
| | | CUCACGAGGUGUCUCUCACCCCCACACUGGGACUCGUGUGGCCUGU |
| | | GUGGUGCCACCCUGCUGGGGCCUCCCAAGUUUUGAAAGGCUUUCCU |
| | | CAGCACCUGGGACCCAACAGAGACCAGCUUCUAGCAGCUAAGGAGG |
| | | CCGUUCAGCUGUGACGAAGGCCUGAAGCACAGGAUUAGGACUGAAG |
| | | CGAUGAUGUCCCCUUCCCUACUUCCCCUUGGGGCUCCCUGUGUCAG |
| | | GGCACAGACUAGGUCUUGUGGCUGGUCUGGCUUGCGGCGCGAGGAU |
| | | GGUUCUCUCUGGUCAUAGCCCGAAGUCUCAUGGCAGUCCCAAAGGA |
| | | GGCUUACAACUCCUGCAUCACAAGAAAAAGGAAGCCACUGCCAGCU |
| | | GGGGGGAUCUGCAGCUCCCAGAAGCUCCGUGAGCCUCAGCCACCCC |
| | | UCAGACUGGGUUCCUCUCCAAGCUCGCCCUCUGGAGGGGCAGCGCA |
| | | GCCUCCCACCAAGGGCCCUGCGACCACAGCAGGGAUUGGGAUGAAU |
| | | UGCCUGUCCUGGAUCUGCUCUAGAGGCCCAAGCUGCCUGCCUGAGG |
| | | AAGGAUGACUUGACAAGUCAGGAGACACUGUUCCCAAAGCCUUGAC |
| | | CAGAGCACCUCAGCCCGCUGACCUUGCACAAACUCCAUCUGCUGCC |
| | | AUGAGAAAAGGGAAGCCGCCUUUGCAAAACAUUGCUGCCUAAAGAA |
| | | ACUCAGCAGCCUCAGGCCCAAUUCUGCCACUUCUGGUUUGGGUACA |
| | | GUUAAAGGCAACCCUGAGGGACUUGGCAGUAGAAAUCCAGGGCCUC |
| | | CCCUGGGGCUGGCAGCUUCGUGUGCAGCUAGAGCUUUACCUGAAAG |
| | | GAAGUCUCUGGGCCCAGAACUCUCCACCAAGAGCCUCCCUGCCGUU |
| | | CGCUGAGUCCCAGCAAUUCUCCUAAGUUGAAGGGAUCUGAGAAGGA |
| | | GAAGGAAAUGUGGGGUAGAUUUGGUGGUGGUUAGAGAUAUGCCCC |
| | | CCUCAUUACUGCCAACAGUUUCGGCUGCAUUUCUUCACGCACCUCG |
| | | GUUCCUCUUCCUGAAGUUCUUGUGCCCUGCUCUUCAGCACCAUGGG |
| | | CCUUCUUAUACGGAAGGCUCUGGGAUCUCCCCCUUGUGGGGCAGGC |
| | | UCUUGGGGCCAGCCUAAGAUCAUGGUUUAGGGUGAUCAGUGCUGGC |
| | | AGAUAAAUUGAAAAGGCACGCUGGCUUGUGAUCUUAAAUGAGGAC |
| | | AAUCCCCCCAGGGCUGGGCACUCCUCCCCUCCCCUCACUUCUCCCAC |
| | | CUGCAGAGCCAGUGUCCUUGGGUGGGCUAGAUAGGAUAUACUGUAU |
| | | GCCGGCUCCUUCAAGCUGCUGACUCACUUUAUCAAUAGUUCCAUUU |
| | | AAAUUGACUUCAGUGGUGAGACUGUAUCCUGUUUGCUAUUGCUUG |
| | | UUGUGCUAUGGGGGGAGGGGGGAGGAAUGUGUAAGAUAGUUAACA |
| | | UGGGCAAAGGGAGAUCUUGGGGUGCAGCACUUAAACUGCCUCGUAA |
| | | CCCUUUUCAUGAUUUCAACCACAUUUGCUAGAGGGAGGGAGCAGCC |
| | | ACGGAGUUAGAGGCCCUUGGGGUUUCUCUUUUCCACUGACAGGCUU |
| | | UCCCAGGCAGCUGGCUAGUUCAUUCCCUCCCCAGCCAGGUGCAGGC |
| | | GUAGGAAUAUGGACAUCUGGUUGCUUUGGCCUGCUGCCCUCUUUCA |
| | | GGGGUCCUAAGCCCACAAUCAUGCCUCCCUAAGACCUUGGCAUCCU |
| | | UCCCUCUAAGCCGUUGGCACCUCUGUGCCACCUCUCACACUGGCUCC |
| | | AGACACACAGCCUGUGCUUUUGGAGCUGAGAUCACUCGCUUCACCC |
| | | UCCUCAUCUUUGUUCUCCAAGUAAAGCCACGAGGUCGGGGCGAGGG |
| | | CAGAGGUGAUCACCUGCGUGUCCCAUCUACAGACCUGCAGCUUCAU |
| | | AAAACUUCUGAUUUCUCUUCAGCUUUGAAAAGGGUUACCCUGGGCA |
| | | CUGGCCUAGAGCCUCACCUCCUAAUAGACUUAGCCCCAUGAGUUUG |
| | | CCAUGUUGAGCAGGACUAUUUCUGGCACUUGCAAGUCCCAUGAUUU |
| | | CUUCGGUAAUUCUGAGGGUGGGGGGAGGGACAUGAAAUCAUCUUA |
| | | GCUUAGCUUUCUGUCUGUGAAUGUCUAUAUAGUGUAUUGUGUGUU |
| | | UUAACAAAUGAUUUACACUGACUGUUGCUGUAAAAGUGAAUUUGG |
| | | AAAUAAAGUUAUUACUCUGAUUAAA |
| 22 | 7 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GCUGAAGAAGCAGGCAUUGGAGACACCCCCAGCCUGGAAGACGAAG |
| | | CUGCUGGUCACGUGACCCAAGCUCGCAUGGUCAGUAAAAGCAAAGA |
| | | CGGGACUGGAAGCGAUGACAAAAAAGCCAAGGGGGCUGAUGGUAA |
| | | AACGAAGAUCGCCACACCGCGGGGAGCAGCCCCUCCAGGCCAGAAG |
| | | GGCCAGGCCAACGCCACCAGGAUUCCAGCAAAAACCCCGCCCGCUCC |
| | | AAAGACACCACCCAGCUCUGGUGAACCUCCAAAAUCAGGGGAUCGC |
| | | AGCGGCUACAGCAGCCCCGGCUCCCCAGGCACUCCCGGCAGCCGCUC |
| | | CCGCACCCCGUCCCUUCCAACCCCACCCACCCGGGAGCCCAAGAAGG |
| | | UGGCAGUGGUCCGUACUCCACCCAAGUCGCCGUCUUCCGCCAAGAG |
| | | CCGCCUGCAGACAGCCCCCGUGCCCAUGCCAGACCUGAAGAAUGUC |
| | | AAGUCCAAGAUCGGCUCCACUGAGAACCUGAAGCACCAGCCGGGAG |
| | | GCGGGAAGGUGCAAAUAGUCUACAAACCAGUUGACCUGAGCAAGGU |
| | | GACCUCCAAGUGUGGCUCAUUAGGCAACAUCCAUCAUAAACCAGGA |
| | | GGUGGCCAGGUGGAAGUAAAAUCUGAGAAGCUUGACUUCAAGGAC |
| | | AGAGUCCAGUCGAAGAUUGGGUCCCUGGACAAUAUCACCCACGUCC |
| | | CUGGCGGAGGAAAUAAAAAGAUUGAAACCCACAAGCUGACCUUCCG |
| | | CGAGAACGCCAAAGCCAAGACAGACCACGGGGCGGAGAUCGUGUAC |
| | | AAGUCGCCAGUGGUGUCUGGGGACACGUCUCCACGGCAUCUCAGCA |
| | | AUGUCUCCUCCACCGGCAGCAUCGACAUGGUAGACUCGCCCCAGCU |
| | | CGCCACGCUAGCUGACGAGGUGUCUGCCUCCCUGGCCAAGCAGGGU |
| | | UUGUGAUCAGGCCCCUGGGGCGGUCAAUAAUUGUGGAGAGGAGAG |
| | | AAUGAGAGAGUGUGGAAAAAAAAAGAAUAAUGACCCGGCCCCCGCC |
| | | CUCUGCCCCCAGCUGCUCCUCGCAGUUCGGUUAAUUGGUUAAUCAC |
| | | UUAACCUGCUUUUGUCACUCGGCUUUGGCUCGGGACUUCAAAAUCA |
| | | GUGAUGGGAGUAAGAGCAAAUUUCAUCUUUCCAAAUUGAUGGGUG |
| | | GGCUAGUAAUAAAAUAUUUAAAAAAAAACAUUCAAAAACAUGGCC |
| | | ACAUCCAACAUUUCCUCAGGCAAUUCCUUUUGAUUCUUUUUUCUUC |
| | | CCCCUCCAUGUAGAAGAGGGAGAAGGAGAGGCUCUGAAAGCUGCUU |
| | | CUGGGGGAUUUCAAGGGACUGGGGGUGCCAACCACCUCUGGCCCUG |
| | | UUGUGGGGGUGUCACAGAGGCAGUGGCAGCAACAAAGGAUUUGAA |
| | | ACUUGGUGUGUUCGUGGAGCCACAGGCAGACGAUGUCAACCUUGUG |
| | | UGAGUGUGACGGGGGUUGGGGUGGGGCGGGAGGCCACGGGGGAGG |
| | | CCGAGGCAGGGGCUGGGCAGAGGGGAGAGGAAGCACAAGAAGUGG |
| | | GAGUGGGAGAGGAAGCCACGUGCUGGAGAGUAGACAUCCCCCUCCU |
| | | UGCCGCUGGGAGAGCCAAGGCCUAUGCCACCUGCAGCGUCUGAGCG |
| | | GCCGCCUGUCCUUGGUGGCCGGGGGUGGGGGCCUGCUGUGGGUCAG |
| | | UGUGCCACCCUCUGCAGGGCAGCCUGUGGGAGAAGGGACAGCGGGU |
| | | AAAAAGAGAAGGCAAGCUGGCAGGAGGGUGGCACUUCGUGGAUGA |
| | | CCUCCUUAGAAAAGACUGACCUUGAUGUCUUGAGAGCGCUGGCCUC |
| | | UUCCUCCCUCCCUGCAGGGUAGGGGGCCUGAGUUGAGGGGCUUCCC |
| | | UCUGCUCCACAGAAACCCUGUUUUAUUGAGUUCUGAAGGUUGGAAC |
| | | UGCUGCCAUGAUUUUGGCCACUUUGCAGACCUGGGACUUUAGGGCU |
| | | AACCAGUUCUCUUUGUAAGGACUUGUGCCUCUUGGGAGACGUCCAC |
| | | CCGUUUCCAAGCCUGGGCCACUGGCAUCUCUGGAGUGUGUGGGGGU |
| | | CUGGGAGGCAGGUCCCGAGCCCCCUGUCCUUCCCACGGCCACUGCA |
| | | GUCACCCCGUCUGCGCCGCUGUGCUGUUGUCUGCCGUGAGAGCCCA |
| | | AUCACUGCCUAUACCCCUCAUCACACGUCACAAUGUCCCGAAUUCC |
| | | CAGCCUCACCACCCCUUCUCAGUAAUGACCCUGGUUGGUUGCAGGA |
| | | GGUACCUACUCCAUACUGAGGGUGAAAUUAAGGGAAGGCAAAGUCC |
| | | AGGCACAAGAGUGGGACCCCAGCCUCUCACUCUCAGUUCCACUCAU |
| | | CCAACUGGGACCCUCACCACGAAUCUCAUGAUCUGAUUCGGUUCCC |
| | | UGUCUCCUCCUCCCGUCACAGAUGUGAGCCAGGGCACUGCUCAGCU |
| | | GUGACCCUAGGUGUUUCUGCCUUGUUGACAUGGAGAGAGCCCUUUC |
| | | CCCUGAGAAGGCCUGGCCCCUUCCUGUGCUGAGCCCACAGCAGCAG |
| | | GCUGGGUGUCUUGGUUGUCAGUGGUGGCACCAGGAUGGAAGGGCA |
| | | AGGCACCCAGGGCAGGCCCACAGUCCCGCUGUCCCCCACUUGCACCC |
| | | UAGCUUGUAGCUGCCAACCUCCCAGACAGCCCAGCCCGCUGCUCAG |
| | | CUCCACAUGCAUAGUAUCAGCCCUCCACACCCGACAAAGGGGAACA |
| | | CACCCCCUUGGAAAUGGUUCUUUUCCCCCAGUCCCAGCUGGAAGCC |
| | | AUGCUGUCUGUUCUGCUGGAGCAGCUGAACAUAUACAUAGAUGUUG |
| | | CCCUGCCCUCCCCAUCUGCACCCUGUUGAGUUGUAGUUGGAUUUGU |
| | | CUGUUUAUGCUUGGAUUCACCAGAGUGACUAUGAUAGUGAAAAGA |
| | | AAAAAAAAAAAAAAAAAGGACGCAUGUAUCUUGAAAUGCUUGUAA |
| | | AGAGGUUUCUAACCCACCCUCACGAGGUGUCUCUCACCCCCACACU |
| | | GGGACUCGUGUGGCCUGUGUGGUGCCACCCUGCUGGGGCCUCCCAA |
| | | GUUUUGAAAGGCUUUCCUCAGCACCUGGGACCCAACAGAGACCAGC |
| | | UUCUAGCAGCUAAGGAGGCCGUUCAGCUGUGACGAAGGCCUGAAGC |
| | | ACAGGAUUAGGACUGAAGCGAUGAUGUCCCCUUCCCUACUUCCCCU |
| | | UGGGGCUCCCUGUGUCAGGGCACAGACUAGGUCUUGUGGCUGGUCU |
| | | GGCUUGCGGCGCGAGGAUGGUUCUCUCUGGUCAUAGCCCGAAGUCU |
| | | CAUGGCAGUCCCAAAGGAGGCUUACAACUCCUGCAUCACAAGAAAA |
| | | AGGAAGCCACUGCCAGCUGGGGGGAUCUGCAGCUCCCAGAAGCUCC |
| | | GUGAGCCUCAGCCACCCCUCAGACUGGGUUCCUCUCCAAGCUCGCCC |
| | | UCUGGAGGGGCAGCGCAGCCUCCCACCAAGGGCCCUGCGACCACAG |
| | | CAGGGAUUGGGAUGAAUUGCCUGUCCUGGAUCUGCUCUAGAGGCCC |
| | | AAGCUGCCUGCCUGAGGAAGGAUGACUUGACAAGUCAGGAGACACU |
| | | GUUCCCAAAGCCUUGACCAGAGCACCUCAGCCCGCUGACCUUGCAC |
| | | AAACUCCAUCUGCUGCCAUGAGAAAAGGGAAGCCGCCUUUGCAAAA |
| | | CAUUGCUGCCUAAAGAAACUCAGCAGCCUCAGGCCCAAUUCUGCCA |
| | | CUUCUGGUUUGGGUACAGUUAAAGGCAACCCUGAGGGACUUGGCAG |
| | | UAGAAAUCCAGGGCCUCCCCUGGGGCUGGCAGCUUCGUGUGCAGCU |
| | | AGAGCUUUACCUGAAAGGAAGUCUCUGGGCCCAGAACUCUCCACCA |
| | | AGAGCCUCCCUGCCGUUCGCUGAGUCCCAGCAAUUCUCCUAAGUUG |
| | | AAGGGAUCUGAGAAGGAGAAGGAAAUGUGGGGUAGAUUUGGUGGU |
| | | GGUUAGAGAUAUGCCCCCCUCAUUACUGCCAACAGUUUCGGCUGCA |
| | | UUUCUUCACGCACCUCGGUUCCUCUUCCUGAAGUUCUUGUGCCCUG |
| | | CUCUUCAGCACCAUGGGCCUUCUUAUACGGAAGGCUCUGGGAUCUC |
| | | CCCCUUGUGGGGCAGGCUCUUGGGGCCAGCCUAAGAUCAUGGUUUA |
| | | GGGUGAUCAGUGCUGGCAGAUAAAUUGAAAAGGCACGCUGGCUUG |
| | | UGAUCUUAAAUGAGGACAAUCCCCCCAGGGCUGGGCACUCCUCCCC |
| | | UCCCCUCACUUCUCCCACCUGCAGAGCCAGUGUCCUUGGGUGGGCU |
| | | AGAUAGGAUAUACUGUAUGCCGGCUCCUUCAAGCUGCUGACUCACU |
| | | UUAUCAAUAGUUCCAUUUAAAUUGACUUCAGUGGUGAGACUGUAU |
| | | CCUGUUUGCUAUUGCUUGUUGUGCUAUGGGGGGAGGGGGGAGGAA |
| | | UGUGUAAGAUAGUUAACAUGGGCAAAGGGAGAUCUUGGGGUGCAG |
| | | CACUUAAACUGCCUCGUAACCCUUUUCAUGAUUUCAACCACAUUUG |
| | | CUAGAGGGAGGGAGCAGCCACGGAGUUAGAGGCCCUUGGGGUUUCU |
| | | CUUUUCCACUGACAGGCUUUCCCAGGCAGCUGGCUAGUUCAUUCCC |
| | | UCCCCAGCCAGGUGCAGGCGUAGGAAUAUGGACAUCUGGUUGCUUU |
| | | GGCCUGCUGCCCUCUUUCAGGGGUCCUAAGCCCACAAUCAUGCCUC |
| | | CCUAAGACCUUGGCAUCCUUCCCUCUAAGCCGUUGGCACCUCUGUG |
| | | CCACCUCUCACACUGGCUCCAGACACACAGCCUGUGCUUUUGGAGC |
| | | UGAGAUCACUCGCUUCACCCUCCUCAUCUUUGUUCUCCAAGUAAAG |
| | | CCACGAGGUCGGGGCGAGGGCAGAGGUGAUCACCUGCGUGUCCCAU |
| | | CUACAGACCUGCAGCUUCAUAAAACUUCUGAUUUCUCUUCAGCUUU |
| | | GAAAAGGGUUACCCUGGGCACUGGCCUAGAGCCUCACCUCCUAAUA |
| | | GACUUAGCCCCAUGAGUUUGCCAUGUUGAGCAGGACUAUUUCUGGC |
| | | ACUUGCAAGUCCCAUGAUUUCUUCGGUAAUUCUGAGGGUGGGGGGA |
| | | GGGACAUGAAAUCAUCUUAGCUUAGCUUUCUGUCUGUGAAUGUCUA |
| | | UAUAGUGUAUUGUGUGUUUUAACAAAUGAUUUACACUGACUGUUG |
| | | CUGUAAAAGUGAAUUUGGAAAUAAAGUUAUUACUCUGAUUAAA |
| 23 | 8 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GAUGUGACAGCACCCUUAGUGGAUGAGGGAGCUCCCGGCAAGCAGG |
| | | CUGCCGCGCAGCCCCACACGGAGAUCCCAGAAGGAACCACAGCUGA |
| | | AGAAGCAGGCAUUGGAGACACCCCCAGCCUGGAAGACGAAGCUGCU |
| | | GGUCACGUGACCCAAGCUCGCAUGGUCAGUAAAAGCAAAGACGGGA |
| | | CUGGAAGCGAUGACAAAAAAGCCAAGGGGGCUGAUGGUAAAACGA |
| | | AGAUCGCCACACCGCGGGGAGCAGCCCCUCCAGGCCAGAAGGGCCA |
| | | GGCCAACGCCACCAGGAUUCCAGCAAAAACCCCGCCCGCUCCAAAG |
| | | ACACCACCCAGCUCUGGUGAACCUCCAAAAUCAGGGGAUCGCAGCG |
| | | GCUACAGCAGCCCCGGCUCCCCAGGCACUCCCGGCAGCCGCUCCCGC |
| | | ACCCCGUCCCUUCCAACCCCACCCACCCGGGAGCCCAAGAAGGUGGC |
| | | AGUGGUCCGUACUCCACCCAAGUCGCCGUCUUCCGCCAAGAGCCGC |
| | | CUGCAGACAGCCCCCGUGCCCAUGCCAGACCUGAAGAAUGUCAAGU |
| | | CCAAGAUCGGCUCCACUGAGAACCUGAAGCACCAGCCGGGAGGCGG |
| | | GAAGGUGCAAAUAGUCUACAAACCAGUUGACCUGAGCAAGGUGACC |
| | | UCCAAGUGUGGCUCAUUAGGCAACAUCCAUCAUAAACCAGGAGGUG |
| | | GCCAGGUGGAAGUAAAAUCUGAGAAGCUUGACUUCAAGGACAGAG |
| | | UCCAGUCGAAGAUUGGGUCCCUGGACAAUAUCACCCACGUCCCUGG |
| | | CGGAGGAAAUAAAAAGAUUGAAACCCACAAGCUGACCUUCCGCGAG |
| | | AACGCCAAAGCCAAGACAGACCACGGGGCGGAGAUCGUGUACAAGU |
| | | CGCCAGUGGUGUCUGGGGACACGUCUCCACGGCAUCUCAGCAAUGU |
| | | CUCCUCCACCGGCAGCAUCGACAUGGUAGACUCGCCCCAGCUCGCCA |
| | | CGCUAGCUGACGAGGUGUCUGCCUCCCUGGCCAAGCAGGGUUUGUG |
| | | AUCAGGCCCCUGGGGCGGUCAAUAAUUGUGGAGAGGAGAGAAUGA |
| | | GAGAGUGUGGAAAAAAAAAGAAUAAUGACCCGGCCCCCGCCCUCUG |
| | | CCCCCAGCUGCUCCUCGCAGUUCGGUUAAUUGGUUAAUCACUUAAC |
| | | CUGCUUUUGUCACUCGGCUUUGGCUCGGGACUUCAAAAUCAGUGAU |
| | | GGGAGUAAGAGCAAAUUUCAUCUUUCCAAAUUGAUGGGUGGGCUA |
| | | GUAAUAAAAUAUUUAAAAAAAAACAUUCAAAAACAUGGCCACAUCC |
| | | AACAUUUCCUCAGGCAAUUCCUUUUGAUUCUUUUUUCUUCCCCCUC |
| | | CAUGUAGAAGAGGGAGAAGGAGAGGCUCUGAAAGCUGCUUCUGGG |
| | | GGAUUUCAAGGGACUGGGGGUGCCAACCACCUCUGGCCCUGUUGUG |
| | | GGGGUGUCACAGAGGCAGUGGCAGCAACAAAGGAUUUGAAACUUG |
| | | GUGUGUUCGUGGAGCCACAGGCAGACGAUGUCAACCUUGUGUGAGU |
| | | GUGACGGGGGUUGGGGUGGGGCGGGAGGCCACGGGGGAGGCCGAG |
| | | GCAGGGGCUGGGCAGAGGGGAGAGGAAGCACAAGAAGUGGGAGUG |
| | | GGAGAGGAAGCCACGUGCUGGAGAGUAGACAUCCCCCUCCUUGCCG |
| | | CUGGGAGAGCCAAGGCCUAUGCCACCUGCAGCGUCUGAGCGGCCGC |
| | | CUGUCCUUGGUGGCCGGGGGUGGGGGCCUGCUGUGGGUCAGUGUGC |
| | | CACCCUCUGCAGGGCAGCCUGUGGGAGAAGGGACAGCGGGUAAAAA |
| | | GAGAAGGCAAGCUGGCAGGAGGGUGGCACUUCGUGGAUGACCUCCU |
| | | UAGAAAAGACUGACCUUGAUGUCUUGAGAGCGCUGGCCUCUUCCUC |
| | | CCUCCCUGCAGGGUAGGGGGCCUGAGUUGAGGGGCUUCCCUCUGCU |
| | | CCACAGAAACCCUGUUUUAUUGAGUUCUGAAGGUUGGAACUGCUGC |
| | | CAUGAUUUUGGCCACUUUGCAGACCUGGGACUUUAGGGCUAACCAG |
| | | UUCUCUUUGUAAGGACUUGUGCCUCUUGGGAGACGUCCACCCGUUU |
| | | CCAAGCCUGGGCCACUGGCAUCUCUGGAGUGUGUGGGGGUCUGGGA |
| | | GGCAGGUCCCGAGCCCCCUGUCCUUCCCACGGCCACUGCAGUCACCC |
| | | CGUCUGCGCCGCUGUGCUGUUGUCUGCCGUGAGAGCCCAAUCACUG |
| | | CCUAUACCCCUCAUCACACGUCACAAUGUCCCGAAUUCCCAGCCUCA |
| | | CCACCCCUUCUCAGUAAUGACCCUGGUUGGUUGCAGGAGGUACCUA |
| | | CUCCAUACUGAGGGUGAAAUUAAGGGAAGGCAAAGUCCAGGCACAA |
| | | GAGUGGGACCCCAGCCUCUCACUCUCAGUUCCACUCAUCCAACUGG |
| | | GACCCUCACCACGAAUCUCAUGAUCUGAUUCGGUUCCCUGUCUCCU |
| | | CCUCCCGUCACAGAUGUGAGCCAGGGCACUGCUCAGCUGUGACCCU |
| | | AGGUGUUUCUGCCUUGUUGACAUGGAGAGAGCCCUUUCCCCUGAGA |
| | | AGGCCUGGCCCCUUCCUGUGCUGAGCCCACAGCAGCAGGCUGGGUG |
| | | UCUUGGUUGUCAGUGGUGGCACCAGGAUGGAAGGGCAAGGCACCCA |
| | | GGGCAGGCCCACAGUCCCGCUGUCCCCCACUUGCACCCUAGCUUGU |
| | | AGCUGCCAACCUCCCAGACAGCCCAGCCCGCUGCUCAGCUCCACAUG |
| | | CAUAGUAUCAGCCCUCCACACCCGACAAAGGGGAACACACCCCCUU |
| | | GGAAAUGGUUCUUUUCCCCCAGUCCCAGCUGGAAGCCAUGCUGUCU |
| | | GUUCUGCUGGAGCAGCUGAACAUAUACAUAGAUGUUGCCCUGCCCU |
| | | CCCCAUCUGCACCCUGUUGAGUUGUAGUUGGAUUUGUCUGUUUAUG |
| | | CUUGGAUUCACCAGAGUGACUAUGAUAGUGAAAAGAAAAAAAAAA |
| | | AAAAAAAAGGACGCAUGUAUCUUGAAAUGCUUGUAAAGAGGUUUC |
| | | UAACCCACCCUCACGAGGUGUCUCUCACCCCCACACUGGGACUCGU |
| | | GUGGCCUGUGUGGUGCCACCCUGCUGGGGCCUCCCAAGUUUUGAAA |
| | | GGCUUUCCUCAGCACCUGGGACCCAACAGAGACCAGCUUCUAGCAG |
| | | CUAAGGAGGCCGUUCAGCUGUGACGAAGGCCUGAAGCACAGGAUUA |
| | | GGACUGAAGCGAUGAUGUCCCCUUCCCUACUUCCCCUUGGGGCUCC |
| | | CUGUGUCAGGGCACAGACUAGGUCUUGUGGCUGGUCUGGCUUGCGG |
| | | CGCGAGGAUGGUUCUCUCUGGUCAUAGCCCGAAGUCUCAUGGCAGU |
| | | CCCAAAGGAGGCUUACAACUCCUGCAUCACAAGAAAAAGGAAGCCA |
| | | CUGCCAGCUGGGGGGAUCUGCAGCUCCCAGAAGCUCCGUGAGCCUC |
| | | AGCCACCCCUCAGACUGGGUUCCUCUCCAAGCUCGCCCUCUGGAGG |
| | | GGCAGCGCAGCCUCCCACCAAGGGCCCUGCGACCACAGCAGGGAUU |
| | | GGGAUGAAUUGCCUGUCCUGGAUCUGCUCUAGAGGCCCAAGCUGCC |
| | | UGCCUGAGGAAGGAUGACUUGACAAGUCAGGAGACACUGUUCCCAA |
| | | AGCCUUGACCAGAGCACCUCAGCCCGCUGACCUUGCACAAACUCCA |
| | | UCUGCUGCCAUGAGAAAAGGGAAGCCGCCUUUGCAAAACAUUGCUG |
| | | CCUAAAGAAACUCAGCAGCCUCAGGCCCAAUUCUGCCACUUCUGGU |
| | | UUGGGUACAGUUAAAGGCAACCCUGAGGGACUUGGCAGUAGAAAUC |
| | | CAGGGCCUCCCCUGGGGCUGGCAGCUUCGUGUGCAGCUAGAGCUUU |
| | | ACCUGAAAGGAAGUCUCUGGGCCCAGAACUCUCCACCAAGAGCCUC |
| | | CCUGCCGUUCGCUGAGUCCCAGCAAUUCUCCUAAGUUGAAGGGAUC |
| | | UGAGAAGGAGAAGGAAAUGUGGGGUAGAUUUGGUGGUGGUUAGAG |
| | | AUAUGCCCCCCUCAUUACUGCCAACAGUUUCGGCUGCAUUUCUUCA |
| | | CGCACCUCGGUUCCUCUUCCUGAAGUUCUUGUGCCCUGCUCUUCAG |
| | | CACCAUGGGCCUUCUUAUACGGAAGGCUCUGGGAUCUCCCCCUUGU |
| | | GGGGCAGGCUCUUGGGGCCAGCCUAAGAUCAUGGUUUAGGGUGAUC |
| | | AGUGCUGGCAGAUAAAUUGAAAAGGCACGCUGGCUUGUGAUCUUA |
| | | AAUGAGGACAAUCCCCCCAGGGCUGGGCACUCCUCCCCUCCCCUCAC |
| | | UUCUCCCACCUGCAGAGCCAGUGUCCUUGGGUGGGCUAGAUAGGAU |
| | | AUACUGUAUGCCGGCUCCUUCAAGCUGCUGACUCACUUUAUCAAUA |
| | | GUUCCAUUUAAAUUGACUUCAGUGGUGAGACUGUAUCCUGUUUGCU |
| | | AUUGCUUGUUGUGCUAUGGGGGGAGGGGGGAGGAAUGUGUAAGAU |
| | | AGUUAACAUGGGCAAAGGGAGAUCUUGGGGUGCAGCACUUAAACU |
| | | GCCUCGUAACCCUUUUCAUGAUUUCAACCACAUUUGCUAGAGGGAG |
| | | GGAGCAGCCACGGAGUUAGAGGCCCUUGGGGUUUCUCUUUUCCACU |
| | | GACAGGCUUUCCCAGGCAGCUGGCUAGUUCAUUCCCUCCCCAGCCA |
| | | GGUGCAGGCGUAGGAAUAUGGACAUCUGGUUGCUUUGGCCUGCUGC |
| | | CCUCUUUCAGGGGUCCUAAGCCCACAAUCAUGCCUCCCUAAGACCU |
| | | UGGCAUCCUUCCCUCUAAGCCGUUGGCACCUCUGUGCCACCUCUCA |
| | | CACUGGCUCCAGACACACAGCCUGUGCUUUUGGAGCUGAGAUCACU |
| | | CGCUUCACCCUCCUCAUCUUUGUUCUCCAAGUAAAGCCACGAGGUC |
| | | GGGGCGAGGGCAGAGGUGAUCACCUGCGUGUCCCAUCUACAGACCU |
| | | GCAGCUUCAUAAAACUUCUGAUUUCUCUUCAGCUUUGAAAAGGGUU |
| | | ACCCUGGGCACUGGCCUAGAGCCUCACCUCCUAAUAGACUUAGCCC |
| | | CAUGAGUUUGCCAUGUUGAGCAGGACUAUUUCUGGCACUUGCAAGU |
| | | CCCAUGAUUUCUUCGGUAAUUCUGAGGGUGGGGGGAGGGACAUGA |
| | | AAUCAUCUUAGCUUAGCUUUCUGUCUGUGAAUGUCUAUAUAGUGU |
| | | AUUGUGUGUUUUAACAAAUGAUUUACACUGACUGUUGCUGUAAAA |
| | | GUGAAUUUGGAAAUAAAGUUAUUACUCUGAUUAAA |
| 24 | 9 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GCUGAAGAAGCAGGCAUUGGAGACACCCCCAGCCUGGAAGACGAAG |
| | | CUGCUGGUCACGUGACCCAAGAGGAGUUGAGAGUUCCGGGCCGGCA |
| | | GAGGAAGGCGCCUGAAAGGCCCCUGGCCAAUGAGAUUAGCGCCCAC |
| | | GUCCAGCCUGGACCCUGCGGAGAGGCCUCUGGGGUCUCUGGGCCGU |
| | | GCCUCGGGGAGAAAGAGCCAGAAGCUCCCGUCCCGCUGACCGCGAG |
| | | CCUUCCUCAGCACCGUCCCGUUUGCCCAGCGCCUCCUCCAACAGGAG |
| | | GCCCUCAGGAGCCCUCCCUGGAGUGGGGACAAAAAGGCGGGGACUG |
| | | GGCCGAGAAGGGUCCGGCCUUUCCGAAGCCCGCCACCACUGCGUAU |
| | | CUCCACACAGAGCCUGAAAGUGGUAAGGUGGUCCAGGAAGGCUUCC |
| | | UCCGAGAGCCAGGCCCCCCAGGUCUGAGCCACCAGCUCAUGUCCGG |
| | | CAUGCCUGGGGCUCCCCUCCUGCCUGAGGGCCCCAGAGAGGCCACA |
| | | CGCCAACCUUCGGGGACAGGACCUGAGGACACAGAGGGCGGCCGCC |
| | | ACGCCCCUGAGCUGCUCAAGCACCAGCUUCUAGGAGACCUGCACCA |
| | | GGAGGGGCCGCCGCUGAAGGGGGCAGGGGGCAAAGAGAGGCCGGGG |
| | | AGCAAGGAGGAGGUGGAUGAAGACCGCGACGUCGAUGAGUCCUCCC |
| | | CCCAAGACUCCCCUCCCUCCAAGGCCUCCCCAGCCCAAGAUGGGCGG |
| | | CCUCCCCAGACAGCCGCCAGAGAAGCCACCAGCAUCCCAGGCUUCCC |
| | | AGCGGAGGGUGCCAUCCCCCUCCCUGUGGAUUUCCUCUCCAAAGUU |
| | | UCCACAGAGAUCCCAGCCUCAGAGCCCGACGGGCCCAGUGUAGGGC |
| | | GGGCCAAAGGGCAGGAUGCCCCCCUGGAGUUCACGUUUCACGUGGA |
| | | AAUCACACCCAACGUGCAGAAGGAGCAGGCGCACUCGGAGGAGCAU |
| | | UUGGGAAGGGCUGCAUUUCCAGGGGCCCCUGGAGAGGGGCCAGAGG |
| | | CCCGGGGCCCCUCUUUGGGAGAGGACACAAAAGAGGCUGACCUUCC |
| | | AGAGCCCUCUGAAAAGCAGCCUGCUGCUGCUCCGCGGGGGAAGCCC |
| | | GUCAGCCGGGUCCCUCAACUCAAAGCUCGCAUGGUCAGUAAAAGCA |
| | | AAGACGGGACUGGAAGCGAUGACAAAAAAGCCAAGACAUCCACACG |
| | | UUCCUCUGCUAAAACCUUGAAAAAUAGGCCUUGCCUUAGCCCCAAA |
| | | CACCCCACUCCUGGUAGCUCAGACCCUCUGAUCCAACCCUCCAGCCC |
| | | UGCUGUGUGCCCAGAGCCACCUUCCUCUCCUAAAUACGUCUCUUCU |
| | | GUCACUUCCCGAACUGGCAGUUCUGGAGCAAAGGAGAUGAAACUCA |
| | | AGGGGGCUGAUGGUAAAACGAAGAUCGCCACACCGCGGGGAGCAGC |
| | | CCCUCCAGGCCAGAAGGGCCAGGCCAACGCCACCAGGAUUCCAGCA |
| | | AAAACCCCGCCCGCUCCAAAGACACCACCCAGCUCUGGUGAACCUCC |
| | | AAAAUCAGGGGAUCGCAGCGGCUACAGCAGCCCCGGCUCCCCAGGC |
| | | ACUCCCGGCAGCCGCUCCCGCACCCCGUCCCUUCCAACCCCACCCAC |
| | | CCGGGAGCCCAAGAAGGUGGCAGUGGUCCGUACUCCACCCAAGUCG |
| | | CCGUCUUCCGCCAAGAGCCGCCUGCAGACAGCCCCCGUGCCCAUGCC |
| | | AGACCUGAAGAAUGUCAAGUCCAAGAUCGGCUCCACUGAGAACCUG |
| | | AAGCACCAGCCGGGAGGCGGGAAGGUGCAGAUAAUUAAUAAGAAGC |
| | | UGGAUCUUAGCAACGUCCAGUCCAAGUGUGGCUCAAAGGAUAAUAU |
| | | CAAACACGUCCCGGGAGGCGGCAGUGUGCAAAUAGUCUACAAACCA |
| | | GUUGACCUGAGCAAGGUGACCUCCAAGUGUGGCUCAUUAGGCAACA |
| | | UCCAUCAUAAACCAGGAGGUGGCCAGGUGGAAGUAAAAUCUGAGAA |
| | | GCUUGACUUCAAGGACAGAGUCCAGUCGAAGAUUGGGUCCCUGGAC |
| | | AAUAUCACCCACGUCCCUGGCGGAGGAAAUAAAAAGAUUGAAACCC |
| | | ACAAGCUGACCUUCCGCGAGAACGCCAAAGCCAAGACAGACCACGG |
| | | GGCGGAGAUCGUGUACAAGUCGCCAGUGGUGUCUGGGGACACGUCU |
| | | CCACGGCAUCUCAGCAAUGUCUCCUCCACCGGCAGCAUCGACAUGG |
| | | UAGACUCGCCCCAGCUCGCCACGCUAGCUGACGAGGUGUCUGCCUC |
| | | CCUGGCCAAGCAGGGUUUGUGAUCAGGCCCCUGGGGCGGUCAAUAA |
| | | UUGUGGAGAGGAGAGAAUGAGAGAGUGUGGAAAAAAAAAGAAUAA |
| | | UGACCCGGCCCCCGCCCUCUGCCCCCAGCUGCUCCUCGCAGUUCGGU |
| | | UAAUUGGUUAAUCACUUAACCUGCUUUUGUCACUCGGCUUUGGCUC |
| | | GGGACUUCAAAAUCAGUGAUGGGAGUAAGAGCAAAUUUCAUCUUU |
| | | CCAAAUUGAUGGGUGGGCUAGUAAUAAAAUAUUUAAAAAAAAACA |
| | | UUCAAAAACAUGGCCACAUCCAACAUUUCCUCAGGCAAUUCCUUUU |
| | | GAUUCUUUUUUCUUCCCCCUCCAUGUAGAAGAGGGAGAAGGAGAGG |
| | | CUCUGAAAGCUGCUUCUGGGGGAUUUCAAGGGACUGGGGGUGCCAA |
| | | CCACCUCUGGCCCUGUUGUGGGGGUGUCACAGAGGCAGUGGCAGCA |
| | | ACAAAGGAUUUGAAACUUGGUGUGUUCGUGGAGCCACAGGCAGACG |
| | | AUGUCAACCUUGUGUGAGUGUGACGGGGGUUGGGGUGGGGCGGGA |
| | | GGCCACGGGGGAGGCCGAGGCAGGGGCUGGGCAGAGGGGAGAGGAA |
| | | GCACAAGAAGUGGGAGUGGGAGAGGAAGCCACGUGCUGGAGAGUA |
| | | GACAUCCCCCUCCUUGCCGCUGGGAGAGCCAAGGCCUAUGCCACCU |
| | | GCAGCGUCUGAGCGGCCGCCUGUCCUUGGUGGCCGGGGGUGGGGGC |
| | | CUGCUGUGGGUCAGUGUGCCACCCUCUGCAGGGCAGCCUGUGGGAG |
| | | AAGGGACAGCGGGUAAAAAGAGAAGGCAAGCUGGCAGGAGGGUGG |
| | | CACUUCGUGGAUGACCUCCUUAGAAAAGACUGACCUUGAUGUCUUG |
| | | AGAGCGCUGGCCUCUUCCUCCCUCCCUGCAGGGUAGGGGGCCUGAG |
| | | UUGAGGGGCUUCCCUCUGCUCCACAGAAACCCUGUUUUAUUGAGUU |
| | | CUGAAGGUUGGAACUGCUGCCAUGAUUUUGGCCACUUUGCAGACCU |
| | | GGGACUUUAGGGCUAACCAGUUCUCUUUGUAAGGACUUGUGCCUCU |
| | | UGGGAGACGUCCACCCGUUUCCAAGCCUGGGCCACUGGCAUCUCUG |
| | | GAGUGUGUGGGGGUCUGGGAGGCAGGUCCCGAGCCCCCUGUCCUUC |
| | | CCACGGCCACUGCAGUCACCCCGUCUGCGCCGCUGUGCUGUUGUCU |
| | | GCCGUGAGAGCCCAAUCACUGCCUAUACCCCUCAUCACACGUCACA |
| | | AUGUCCCGAAUUCCCAGCCUCACCACCCCUUCUCAGUAAUGACCCU |
| | | GGUUGGUUGCAGGAGGUACCUACUCCAUACUGAGGGUGAAAUUAA |
| | | GGGAAGGCAAAGUCCAGGCACAAGAGUGGGACCCCAGCCUCUCACU |
| | | CUCAGUUCCACUCAUCCAACUGGGACCCUCACCACGAAUCUCAUGA |
| | | UCUGAUUCGGUUCCCUGUCUCCUCCUCCCGUCACAGAUGUGAGCCA |
| | | GGGCACUGCUCAGCUGUGACCCUAGGUGUUUCUGCCUUGUUGACAU |
| | | GGAGAGAGCCCUUUCCCCUGAGAAGGCCUGGCCCCUUCCUGUGCUG |
| | | AGCCCACAGCAGCAGGCUGGGUGUCUUGGUUGUCAGUGGUGGCACC |
| | | AGGAUGGAAGGGCAAGGCACCCAGGGCAGGCCCACAGUCCCGCUGU |
| | | CCCCCACUUGCACCCUAGCUUGUAGCUGCCAACCUCCCAGACAGCCC |
| | | AGCCCGCUGCUCAGCUCCACAUGCAUAGUAUCAGCCCUCCACACCCG |
| | | ACAAAGGGGAACACACCCCCUUGGAAAUGGUUCUUUUCCCCCAGUC |
| | | CCAGCUGGAAGCCAUGCUGUCUGUUCUGCUGGAGCAGCUGAACAUA |
| | | UACAUAGAUGUUGCCCUGCCCUCCCCAUCUGCACCCUGUUGAGUUG |
| | | UAGUUGGAUUUGUCUGUUUAUGCUUGGAUUCACCAGAGUGACUAU |
| | | GAUAGUGAAAAGAAAAAAAAAAAAAAAAAAGGACGCAUGUAUCUU |
| | | GAAAUGCUUGUAAAGAGGUUUCUAACCCACCCUCACGAGGUGUCUC |
| | | UCACCCCCACACUGGGACUCGUGUGGCCUGUGUGGUGCCACCCUGC |
| | | UGGGGCCUCCCAAGUUUUGAAAGGCUUUCCUCAGCACCUGGGACCC |
| | | AACAGAGACCAGCUUCUAGCAGCUAAGGAGGCCGUUCAGCUGUGAC |
| | | GAAGGCCUGAAGCACAGGAUUAGGACUGAAGCGAUGAUGUCCCCUU |
| | | CCCUACUUCCCCUUGGGGCUCCCUGUGUCAGGGCACAGACUAGGUC |
| | | UUGUGGCUGGUCUGGCUUGCGGCGCGAGGAUGGUUCUCUCUGGUCA |
| | | UAGCCCGAAGUCUCAUGGCAGUCCCAAAGGAGGCUUACAACUCCUG |
| | | CAUCACAAGAAAAAGGAAGCCACUGCCAGCUGGGGGGAUCUGCAGC |
| | | UCCCAGAAGCUCCGUGAGCCUCAGCCACCCCUCAGACUGGGUUCCU |
| | | CUCCAAGCUCGCCCUCUGGAGGGGCAGCGCAGCCUCCCACCAAGGG |
| | | CCCUGCGACCACAGCAGGGAUUGGGAUGAAUUGCCUGUCCUGGAUC |
| | | UGCUCUAGAGGCCCAAGCUGCCUGCCUGAGGAAGGAUGACUUGACA |
| | | AGUCAGGAGACACUGUUCCCAAAGCCUUGACCAGAGCACCUCAGCC |
| | | CGCUGACCUUGCACAAACUCCAUCUGCUGCCAUGAGAAAAGGGAAG |
| | | CCGCCUUUGCAAAACAUUGCUGCCUAAAGAAACUCAGCAGCCUCAG |
| | | GCCCAAUUCUGCCACUUCUGGUUUGGGUACAGUUAAAGGCAACCCU |
| | | GAGGGACUUGGCAGUAGAAAUCCAGGGCCUCCCCUGGGGCUGGCAG |
| | | CUUCGUGUGCAGCUAGAGCUUUACCUGAAAGGAAGUCUCUGGGCCC |
| | | AGAACUCUCCACCAAGAGCCUCCCUGCCGUUCGCUGAGUCCCAGCA |
| | | AUUCUCCUAAGUUGAAGGGAUCUGAGAAGGAGAAGGAAAUGUGGG |
| | | GUAGAUUUGGUGGUGGUUAGAGAUAUGCCCCCCUCAUUACUGCCAA |
| | | CAGUUUCGGCUGCAUUUCUUCACGCACCUCGGUUCCUCUUCCUGAA |
| | | GUUCUUGUGCCCUGCUCUUCAGCACCAUGGGCCUUCUUAUACGGAA |
| | | GGCUCUGGGAUCUCCCCCUUGUGGGGCAGGCUCUUGGGGCCAGCCU |
| | | AAGAUCAUGGUUUAGGGUGAUCAGUGCUGGCAGAUAAAUUGAAAA |
| | | GGCACGCUGGCUUGUGAUCUUAAAUGAGGACAAUCCCCCCAGGGCU |
| | | GGGCACUCCUCCCCUCCCCUCACUUCUCCCACCUGCAGAGCCAGUGU |
| | | CCUUGGGUGGGCUAGAUAGGAUAUACUGUAUGCCGGCUCCUUCAAG |
| | | CUGCUGACUCACUUUAUCAAUAGUUCCAUUUAAAUUGACUUCAGUG |
| | | GUGAGACUGUAUCCUGUUUGCUAUUGCUUGUUGUGCUAUGGGGGG |
| | | AGGGGGGAGGAAUGUGUAAGAUAGUUAACAUGGGCAAAGGGAGAU |
| | | CUUGGGGUGCAGCACUUAAACUGCCUCGUAACCCUUUUCAUGAUUU |
| | | CAACCACAUUUGCUAGAGGGAGGGAGCAGCCACGGAGUUAGAGGCC |
| | | CUUGGGGUUUCUCUUUUCCACUGACAGGCUUUCCCAGGCAGCUGGC |
| | | UAGUUCAUUCCCUCCCCAGCCAGGUGCAGGCGUAGGAAUAUGGACA |
| | | UCUGGUUGCUUUGGCCUGCUGCCCUCUUUCAGGGGUCCUAAGCCCA |
| | | CAAUCAUGCCUCCCUAAGACCUUGGCAUCCUUCCCUCUAAGCCGUU |
| | | GGCACCUCUGUGCCACCUCUCACACUGGCUCCAGACACACAGCCUG |
| | | UGCUUUUGGAGCUGAGAUCACUCGCUUCACCCUCCUCAUCUUUGUU |
| | | CUCCAAGUAAAGCCACGAGGUCGGGGCGAGGGCAGAGGUGAUCACC |
| | | UGCGUGUCCCAUCUACAGACCUGCAGCUUCAUAAAACUUCUGAUUU |
| | | CUCUUCAGCUUUGAAAAGGGUUACCCUGGGCACUGGCCUAGAGCCU |
| | | CACCUCCUAAUAGACUUAGCCCCAUGAGUUUGCCAUGUUGAGCAGG |
| | | ACUAUUUCUGGCACUUGCAAGUCCCAUGAUUUCUUCGGUAAUUCUG |
| | | AGGGUGGGGGGAGGGACAUGAAAUCAUCUUAGCUUAGCUUUCUGU |
| | | CUGUGAAUGUCUAUAUAGUGUAUUGUGUGUUUUAACAAAUGAUUU |
| | | ACACUGACUGUUGCUGUAAAAGUGAAUUUGGAAAUAAAGUUAUUA |
| | | CUCUGAUUAAA |
| 25 | 10 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GCUGAAGAAGCAGGCAUUGGAGACACCCCCAGCCUGGAAGACGAAG |
| | | CUGCUGGUCACGUGACCCAAGAGGAGUUGAGAGUUCCGGGCCGGCA |
| | | GAGGAAGGCGCCUGAAAGGCCCCUGGCCAAUGAGAUUAGCGCCCAC |
| | | GUCCAGCCUGGACCCUGCGGAGAGGCCUCUGGGGUCUCUGGGCCGU |
| | | GCCUCGGGGAGAAAGAGCCAGAAGCUCCCGUCCCGCUGACCGCGAG |
| | | CCUUCCUCAGCACCGUCCCGUUUGCCCAGCGCCUCCUCCAACAGGAG |
| | | GCCCUCAGGAGCCCUCCCUGGAGUGGGGACAAAAAGGCGGGGACUG |
| | | GGCCGAGAAGGGUCCGGCCUUUCCGAAGCCCGCCACCACUGCGUAU |
| | | CUCCACACAGAGCCUGAAAGUGGUAAGGUGGUCCAGGAAGGCUUCC |
| | | UCCGAGAGCCAGGCCCCCCAGGUCUGAGCCACCAGCUCAUGUCCGG |
| | | CAUGCCUGGGGCUCCCCUCCUGCCUGAGGGCCCCAGAGAGGCCACA |
| | | CGCCAACCUUCGGGGACAGGACCUGAGGACACAGAGGGCGGCCGCC |
| | | ACGCCCCUGAGCUGCUCAAGCACCAGCUUCUAGGAGACCUGCACCA |
| | | GGAGGGGCCGCCGCUGAAGGGGGCAGGGGGCAAAGAGAGGCCGGGG |
| | | AGCAAGGAGGAGGUGGAUGAAGACCGCGACGUCGAUGAGUCCUCCC |
| | | CCCAAGACUCCCCUCCCUCCAAGGCCUCCCCAGCCCAAGAUGGGCGG |
| | | CCUCCCCAGACAGCCGCCAGAGAAGCCACCAGCAUCCCAGGCUUCCC |
| | | AGCGGAGGGUGCCAUCCCCCUCCCUGUGGAUUUCCUCUCCAAAGUU |
| | | UCCACAGAGAUCCCAGCCUCAGAGCCCGACGGGCCCAGUGUAGGGC |
| | | GGGCCAAAGGGCAGGAUGCCCCCCUGGAGUUCACGUUUCACGUGGA |
| | | AAUCACACCCAACGUGCAGAAGGAGCAGGCGCACUCGGAGGAGCAU |
| | | UUGGGAAGGGCUGCAUUUCCAGGGGCCCCUGGAGAGGGGCCAGAGG |
| | | CCCGGGGCCCCUCUUUGGGAGAGGACACAAAAGAGGCUGACCUUCC |
| | | AGAGCCCUCUGAAAAGCAGCCUGCUGCUGCUCCGCGGGGGAAGCCC |
| | | GUCAGCCGGGUCCCUCAACUCAAAGCUCGCAUGGUCAGUAAAAGCA |
| | | AAGACGGGACUGGAAGCGAUGACAAAAAAGCCAAGGGGGCUGAUG |
| | | GUAAAACGAAGAUCGCCACACCGCGGGGAGCAGCCCCUCCAGGCCA |
| | | GAAGGGCCAGGCCAACGCCACCAGGAUUCCAGCAAAAACCCCGCCC |
| | | GCUCCAAAGACACCACCCAGCUCUGGUGAACCUCCAAAAUCAGGGG |
| | | AUCGCAGCGGCUACAGCAGCCCCGGCUCCCCAGGCACUCCCGGCAGC |
| | | CGCUCCCGCACCCCGUCCCUUCCAACCCCACCCACCCGGGAGCCCAA |
| | | GAAGGUGGCAGUGGUCCGUACUCCACCCAAGUCGCCGUCUUCCGCC |
| | | AAGAGCCGCCUGCAGACAGCCCCCGUGCCCAUGCCAGACCUGAAGA |
| | | AUGUCAAGUCCAAGAUCGGCUCCACUGAGAACCUGAAGCACCAGCC |
| | | GGGAGGCGGGAAGGUGCAAAUAGUCUACAAACCAGUUGACCUGAGC |
| | | AAGGUGACCUCCAAGUGUGGCUCAUUAGGCAACAUCCAUCAUAAAC |
| | | CAGGAGGUGGCCAGGUGGAAGUAAAAUCUGAGAAGCUUGACUUCA |
| | | AGGACAGAGUCCAGUCGAAGAUUGGGUCCCUGGACAAUAUCACCCA |
| | | CGUCCCUGGCGGAGGAAAUAAAAAGAUUGAAACCCACAAGCUGACC |
| | | UUCCGCGAGAACGCCAAAGCCAAGACAGACCACGGGGCGGAGAUCG |
| | | UGUACAAGUCGCCAGUGGUGUCUGGGGACACGUCUCCACGGCAUCU |
| | | CAGCAAUGUCUCCUCCACCGGCAGCAUCGACAUGGUAGACUCGCCC |
| | | CAGCUCGCCACGCUAGCUGACGAGGUGUCUGCCUCCCUGGCCAAGC |
| | | AGGGUUUGUGAUCAGGCCCCUGGGGCGGUCAAUAAUUGUGGAGAG |
| | | GAGAGAAUGAGAGAGUGUGGAAAAAAAAAGAAUAAUGACCCGGCC |
| | | CCCGCCCUCUGCCCCCAGCUGCUCCUCGCAGUUCGGUUAAUUGGUU |
| | | AAUCACUUAACCUGCUUUUGUCACUCGGCUUUGGCUCGGGACUUCA |
| | | AAAUCAGUGAUGGGAGUAAGAGCAAAUUUCAUCUUUCCAAAUUGA |
| | | UGGGUGGGCUAGUAAUAAAAUAUUUAAAAAAAAACAUUCAAAAAC |
| | | AUGGCCACAUCCAACAUUUCCUCAGGCAAUUCCUUUUGAUUCUUUU |
| | | UUCUUCCCCCUCCAUGUAGAAGAGGGAGAAGGAGAGGCUCUGAAAG |
| | | CUGCUUCUGGGGGAUUUCAAGGGACUGGGGGUGCCAACCACCUCUG |
| | | GCCCUGUUGUGGGGGUGUCACAGAGGCAGUGGCAGCAACAAAGGAU |
| | | UUGAAACUUGGUGUGUUCGUGGAGCCACAGGCAGACGAUGUCAACC |
| | | UUGUGUGAGUGUGACGGGGGUUGGGGUGGGGCGGGAGGCCACGGG |
| | | GGAGGCCGAGGCAGGGGCUGGGCAGAGGGGAGAGGAAGCACAAGA |
| | | AGUGGGAGUGGGAGAGGAAGCCACGUGCUGGAGAGUAGACAUCCCC |
| | | CUCCUUGCCGCUGGGAGAGCCAAGGCCUAUGCCACCUGCAGCGUCU |
| | | GAGCGGCCGCCUGUCCUUGGUGGCCGGGGGUGGGGGCCUGCUGUGG |
| | | GUCAGUGUGCCACCCUCUGCAGGGCAGCCUGUGGGAGAAGGGACAG |
| | | CGGGUAAAAAGAGAAGGCAAGCUGGCAGGAGGGUGGCACUUCGUG |
| | | GAUGACCUCCUUAGAAAAGACUGACCUUGAUGUCUUGAGAGCGCUG |
| | | GCCUCUUCCUCCCUCCCUGCAGGGUAGGGGGCCUGAGUUGAGGGGC |
| | | UUCCCUCUGCUCCACAGAAACCCUGUUUUAUUGAGUUCUGAAGGUU |
| | | GGAACUGCUGCCAUGAUUUUGGCCACUUUGCAGACCUGGGACUUUA |
| | | GGGCUAACCAGUUCUCUUUGUAAGGACUUGUGCCUCUUGGGAGACG |
| | | UCCACCCGUUUCCAAGCCUGGGCCACUGGCAUCUCUGGAGUGUGUG |
| | | GGGGUCUGGGAGGCAGGUCCCGAGCCCCCUGUCCUUCCCACGGCCA |
| | | CUGCAGUCACCCCGUCUGCGCCGCUGUGCUGUUGUCUGCCGUGAGA |
| | | GCCCAAUCACUGCCUAUACCCCUCAUCACACGUCACAAUGUCCCGA |
| | | AUUCCCAGCCUCACCACCCCUUCUCAGUAAUGACCCUGGUUGGUUG |
| | | CAGGAGGUACCUACUCCAUACUGAGGGUGAAAUUAAGGGAAGGCAA |
| | | AGUCCAGGCACAAGAGUGGGACCCCAGCCUCUCACUCUCAGUUCCA |
| | | CUCAUCCAACUGGGACCCUCACCACGAAUCUCAUGAUCUGAUUCGG |
| | | UUCCCUGUCUCCUCCUCCCGUCACAGAUGUGAGCCAGGGCACUGCU |
| | | CAGCUGUGACCCUAGGUGUUUCUGCCUUGUUGACAUGGAGAGAGCC |
| | | CUUUCCCCUGAGAAGGCCUGGCCCCUUCCUGUGCUGAGCCCACAGC |
| | | AGCAGGCUGGGUGUCUUGGUUGUCAGUGGUGGCACCAGGAUGGAA |
| | | GGGCAAGGCACCCAGGGCAGGCCCACAGUCCCGCUGUCCCCCACUU |
| | | GCACCCUAGCUUGUAGCUGCCAACCUCCCAGACAGCCCAGCCCGCUG |
| | | CUCAGCUCCACAUGCAUAGUAUCAGCCCUCCACACCCGACAAAGGG |
| | | GAACACACCCCCUUGGAAAUGGUUCUUUUCCCCCAGUCCCAGCUGG |
| | | AAGCCAUGCUGUCUGUUCUGCUGGAGCAGCUGAACAUAUACAUAGA |
| | | UGUUGCCCUGCCCUCCCCAUCUGCACCCUGUUGAGUUGUAGUUGGA |
| | | UUUGUCUGUUUAUGCUUGGAUUCACCAGAGUGACUAUGAUAGUGA |
| | | AAAGAAAAAAAAAAAAAAAAAAGGACGCAUGUAUCUUGAAAUGCU |
| | | UGUAAAGAGGUUUCUAACCCACCCUCACGAGGUGUCUCUCACCCCC |
| | | ACACUGGGACUCGUGUGGCCUGUGUGGUGCCACCCUGCUGGGGCCU |
| | | CCCAAGUUUUGAAAGGCUUUCCUCAGCACCUGGGACCCAACAGAGA |
| | | CCAGCUUCUAGCAGCUAAGGAGGCCGUUCAGCUGUGACGAAGGCCU |
| | | GAAGCACAGGAUUAGGACUGAAGCGAUGAUGUCCCCUUCCCUACUU |
| | | CCCCUUGGGGCUCCCUGUGUCAGGGCACAGACUAGGUCUUGUGGCU |
| | | GGUCUGGCUUGCGGCGCGAGGAUGGUUCUCUCUGGUCAUAGCCCGA |
| | | AGUCUCAUGGCAGUCCCAAAGGAGGCUUACAACUCCUGCAUCACAA |
| | | GAAAAAGGAAGCCACUGCCAGCUGGGGGGAUCUGCAGCUCCCAGAA |
| | | GCUCCGUGAGCCUCAGCCACCCCUCAGACUGGGUUCCUCUCCAAGC |
| | | UCGCCCUCUGGAGGGGCAGCGCAGCCUCCCACCAAGGGCCCUGCGA |
| | | CCACAGCAGGGAUUGGGAUGAAUUGCCUGUCCUGGAUCUGCUCUAG |
| | | AGGCCCAAGCUGCCUGCCUGAGGAAGGAUGACUUGACAAGUCAGGA |
| | | GACACUGUUCCCAAAGCCUUGACCAGAGCACCUCAGCCCGCUGACC |
| | | UUGCACAAACUCCAUCUGCUGCCAUGAGAAAAGGGAAGCCGCCUUU |
| | | GCAAAACAUUGCUGCCUAAAGAAACUCAGCAGCCUCAGGCCCAAUU |
| | | CUGCCACUUCUGGUUUGGGUACAGUUAAAGGCAACCCUGAGGGACU |
| | | UGGCAGUAGAAAUCCAGGGCCUCCCCUGGGGCUGGCAGCUUCGUGU |
| | | GCAGCUAGAGCUUUACCUGAAAGGAAGUCUCUGGGCCCAGAACUCU |
| | | CCACCAAGAGCCUCCCUGCCGUUCGCUGAGUCCCAGCAAUUCUCCU |
| | | AAGUUGAAGGGAUCUGAGAAGGAGAAGGAAAUGUGGGGUAGAUUU |
| | | GGUGGUGGUUAGAGAUAUGCCCCCCUCAUUACUGCCAACAGUUUCG |
| | | GCUGCAUUUCUUCACGCACCUCGGUUCCUCUUCCUGAAGUUCUUGU |
| | | GCCCUGCUCUUCAGCACCAUGGGCCUUCUUAUACGGAAGGCUCUGG |
| | | GAUCUCCCCCUUGUGGGGCAGGCUCUUGGGGCCAGCCUAAGAUCAU |
| | | GGUUUAGGGUGAUCAGUGCUGGCAGAUAAAUUGAAAAGGCACGCU |
| | | GGCUUGUGAUCUUAAAUGAGGACAAUCCCCCCAGGGCUGGGCACUC |
| | | CUCCCCUCCCCUCACUUCUCCCACCUGCAGAGCCAGUGUCCUUGGGU |
| | | GGGCUAGAUAGGAUAUACUGUAUGCCGGCUCCUUCAAGCUGCUGAC |
| | | UCACUUUAUCAAUAGUUCCAUUUAAAUUGACUUCAGUGGUGAGACU |
| | | GUAUCCUGUUUGCUAUUGCUUGUUGUGCUAUGGGGGGAGGGGGGA |
| | | GGAAUGUGUAAGAUAGUUAACAUGGGCAAAGGGAGAUCUUGGGGU |
| | | GCAGCACUUAAACUGCCUCGUAACCCUUUUCAUGAUUUCAACCACA |
| | | UUUGCUAGAGGGAGGGAGCAGCCACGGAGUUAGAGGCCCUUGGGGU |
| | | UUCUCUUUUCCACUGACAGGCUUUCCCAGGCAGCUGGCUAGUUCAU |
| | | UCCCUCCCCAGCCAGGUGCAGGCGUAGGAAUAUGGACAUCUGGUUG |
| | | CUUUGGCCUGCUGCCCUCUUUCAGGGGUCCUAAGCCCACAAUCAUG |
| | | CCUCCCUAAGACCUUGGCAUCCUUCCCUCUAAGCCGUUGGCACCUC |
| | | UGUGCCACCUCUCACACUGGCUCCAGACACACAGCCUGUGCUUUUG |
| | | GAGCUGAGAUCACUCGCUUCACCCUCCUCAUCUUUGUUCUCCAAGU |
| | | AAAGCCACGAGGUCGGGGCGAGGGCAGAGGUGAUCACCUGCGUGUC |
| | | CCAUCUACAGACCUGCAGCUUCAUAAAACUUCUGAUUUCUCUUCAG |
| | | CUUUGAAAAGGGUUACCCUGGGCACUGGCCUAGAGCCUCACCUCCU |
| | | AAUAGACUUAGCCCCAUGAGUUUGCCAUGUUGAGCAGGACUAUUUC |
| | | UGGCACUUGCAAGUCCCAUGAUUUCUUCGGUAAUUCUGAGGGUGGG |
| | | GGGAGGGACAUGAAAUCAUCUUAGCUUAGCUUUCUGUCUGUGAAU |
| | | GUCUAUAUAGUGUAUUGUGUGUUUUAACAAAUGAUUUACACUGAC |
| | | UGUUGCUGUAAAAGUGAAUUUGGAAAUAAAGUUAUUACUCUGAUU |
| | | AAA |
| 26 | 11 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGGUGAACU |
| | | UUGAACCAGGAUGGCUGAGCCCCGCCAGGAGUUCGAAGUGAUGGAA |
| | | GAUCACGCUGGGACGUACGGGUUGGGGGACAGGAAAGAUCAGGGG |
| | | GGCUACACCAUGCACCAAGACCAAGAGGGUGACACGGACGCUGGCC |
| | | UGAAAGAAUCUCCCCUGCAGACCCCCACUGAGGACGGAUCUGAGGA |
| | | ACCGGGCUCUGAAACCUCUGAUGCUAAGAGCACUCCAACAGCGGAA |
| | | GCUGAAGAAGCAGGCAUUGGAGACACCCCCAGCCUGGAAGACGAAG |
| | | CUGCUGGUCACGUGACCCAAGCUCGCAUGGUCAGUAAAAGCAAAGA |
| | | CGGGACUGGAAGCGAUGACAAAAAAGCCAAGGGGGCUGAUGGUAA |
| | | AACGAAGAUCGCCACACCGCGGGGAGCAGCCCCUCCAGGCCAGAAG |
| | | GGCCAGGCCAACGCCACCAGGAUUCCAGCAAAAACCCCGCCCGCUCC |
| | | AAAGACACCACCCAGCUCUGGUGAACCUCCAAAAUCAGGGGAUCGC |
| | | AGCGGCUACAGCAGCCCCGGCUCCCCAGGCACUCCCGGCAGCCGCUC |
| | | CCGCACCCCGUCCCUUCCAACCCCACCCACCCGGGAGCCCAAGAAGG |
| | | UGGCAGUGGUCCGUACUCCACCCAAGUCGCCGUCUUCCGCCAAGAG |
| | | CCGCCUGCAGACAGCCCCCGUGCCCAUGCCAGACCUGAAGAAUGUC |
| | | AAGUCCAAGAUCGGCUCCACUGAGAACCUGAAGCACCAGCCGGGAG |
| | | GCGGGAAGGUGCAAAUAGUCUACAAACCAGUUGACCUGAGCAAGGU |
| | | UGGAACUGCUGCCAUGAUUUUGGCCACUUUGCAGACCUGGGACUUU |
| | | AGGGCUAACCAGUUCUCUUUGUAAGGACUUGUGCCUCUUGGGAGAC |
| | | GUCCACCCGUUUCCAAGCCUGGGCCACUGGCAUCUCUGGAGUGUGU |
| | | GGGGGUCUGGGAGGCAGGUCCCGAGCCCCCUGUCCUUCCCACGGCC |
| | | ACUGCAGUCACCCCGUCUGCGCCGCUGUGCUGUUGUCUGCCGUGAG |
| | | AGCCCAAUCACUGCCUAUACCCCUCAUCACACGUCACAAUGUCCCG |
| | | AAUUCCCAGCCUCACCACCCCUUCUCAGUAAUGACCCUGGUUGGUU |
| | | GCAGGAGGUACCUACUCCAUACUGAGGGUGAAAUUAAGGGAAGGCA |
| | | AAGUCCAGGCACAAGAGUGGGACCCCAGCCUCUCACUCUCAGUUCC |
| | | ACUCAUCCAACUGGGACCCUCACCACGAAUCUCAUGAUCUGAUUCG |
| | | GUUCCCUGUCUCCUCCUCCCGUCACAGAUGUGAGCCAGGGCACUGC |
| | | UCAGCUGUGACCCUAGGUGUUUCUGCCUUGUUGACAUGGAGAGAGC |
| | | CCUUUCCCCUGAGAAGGCCUGGCCCCUUCCUGUGCUGAGCCCACAG |
| | | CAGCAGGCUGGGUGUCUUGGUUGUCAGUGGUGGCACCAGGAUGGAA |
| | | GGGCAAGGCACCCAGGGCAGGCCCACAGUCCCGCUGUCCCCCACUU |
| | | GCACCCUAGCUUGUAGCUGCCAACCUCCCAGACAGCCCAGCCCGCUG |
| | | CUCAGCUCCACAUGCAUAGUAUCAGCCCUCCACACCCGACAAAGGG |
| | | GAACACACCCCCUUGGAAAUGGUUCUUUUCCCCCAGUCCCAGCUGG |
| | | AAGCCAUGCUGUCUGUUCUGCUGGAGCAGCUGAACAUAUACAUAGA |
| | | UGUUGCCCUGCCCUCCCCAUCUGCACCCUGUUGAGUUGUAGUUGGA |
| | | UUUGUCUGUUUAUGCUUGGAUUCACCAGAGUGACUAUGAUAGUGA |
| | | AAAGAAAAAAAAAAAAAAAAAAGGACGCAUGUAUCUUGAAAUGCU |
| | | UGUAAAGAGGUUUCUAACCCACCCUCACGAGGUGUCUCUCACCCCC |
| | | ACACUGGGACUCGUGUGGCCUGUGUGGUGCCACCCUGCUGGGGCCU |
| | | CCCAAGUUUUGAAAGGCUUUCCUCAGCACCUGGGACCCAACAGAGA |
| | | CCAGCUUCUAGCAGCUAAGGAGGCCGUUCAGCUGUGACGAAGGCCU |
| | | GAAGCACAGGAUUAGGACUGAAGCGAUGAUGUCCCCUUCCCUACUU |
| | | CCCCUUGGGGCUCCCUGUGUCAGGGCACAGACUAGGUCUUGUGGCU |
| | | GGUCUGGCUUGCGGCGCGAGGAUGGUUCUCUCUGGUCAUAGCCCGA |
| | | AGUCUCAUGGCAGUCCCAAAGGAGGCUUACAACUCCUGCAUCACAA |
| | | GAAAAAGGAAGCCACUGCCAGCUGGGGGGAUCUGCAGCUCCCAGAA |
| | | GCUCCGUGAGCCUCAGCCACCCCUCAGACUGGGUUCCUCUCCAAGC |
| | | UCGCCCUCUGGAGGGGCAGCGCAGCCUCCCACCAAGGGCCCUGCGA |
| | | CCACAGCAGGGAUUGGGAUGAAUUGCCUGUCCUGGAUCUGCUCUAG |
| | | AGGCCCAAGCUGCCUGCCUGAGGAAGGAUGACUUGACAAGUCAGGA |
| | | GACACUGUUCCCAAAGCCUUGACCAGAGCACCUCAGCCCGCUGACC |
| | | UUGCACAAACUCCAUCUGCUGCCAUGAGAAAAGGGAAGCCGCCUUU |
| | | GCAAAACAUUGCUGCCUAAAGAAACUCAGCAGCCUCAGGCCCAAUU |
| | | CUGCCACUUCUGGUUUGGGUACAGUUAAAGGCAACCCUGAGGGACU |
| | | UGGCAGUAGAAAUCCAGGGCCUCCCCUGGGGCUGGCAGCUUCGUGU |
| | | GCAGCUAGAGCUUUACCUGAAAGGAAGUCUCUGGGCCCAGAACUCU |
| | | CCACCAAGAGCCUCCCUGCCGUUCGCUGAGUCCCAGCAAUUCUCCU |
| | | AAGUUGAAGGGAUCUGAGAAGGAGAAGGAAAUGUGGGGUAGAUUU |
| | | GGUGGUGGUUAGAGAUAUGCCCCCCUCAUUACUGCCAACAGUUUCG |
| | | GCUGCAUUUCUUCACGCACCUCGGUUCCUCUUCCUGAAGUUCUUGU |
| | | GCCCUGCUCUUCAGCACCAUGGGCCUUCUUAUACGGAAGGCUCUGG |
| | | GAUCUCCCCCUUGUGGGGCAGGCUCUUGGGGCCAGCCUAAGAUCAU |
| | | GGUUUAGGGUGAUCAGUGCUGGCAGAUAAAUUGAAAAGGCACGCU |
| | | GGCUUGUGAUCUUAAAUGAGGACAAUCCCCCCAGGGCUGGGCACUC |
| | | CUCCCCUCCCCUCACUUCUCCCACCUGCAGAGCCAGUGUCCUUGGGU |
| | | GGGCUAGAUAGGAUAUACUGUAUGCCGGCUCCUUCAAGCUGCUGAC |
| | | UCACUUUAUCAAUAGUUCCAUUUAAAUUGACUUCAGUGGUGAGACU |
| | | GUAUCCUGUUUGCUAUUGCUUGUUGUGCUAUGGGGGGAGGGGGGA |
| | | GGAAUGUGUAAGAUAGUUAACAUGGGCAAAGGGAGAUCUUGGGGU |
| | | GCAGCACUUAAACUGCCUCGUAACCCUUUUCAUGAUUUCAACCACA |
| | | UUUGCUAGAGGGAGGGAGCAGCCACGGAGUUAGAGGCCCUUGGGGU |
| | | UUCUCUUUUCCACUGACAGGCUUUCCCAGGCAGCUGGCUAGUUCAU |
| | | UCCCUCCCCAGCCAGGUGCAGGCGUAGGAAUAUGGACAUCUGGUUG |
| | | CUUUGGCCUGCUGCCCUCUUUCAGGGGUCCUAAGCCCACAAUCAUG |
| | | CCUCCCUAAGACCUUGGCAUCCUUCCCUCUAAGCCGUUGGCACCUC |
| | | UGUGCCACCUCUCACACUGGCUCCAGACACACAGCCUGUGCUUUUG |
| | | GAGCUGAGAUCACUCGCUUCACCCUCCUCAUCUUUGUUCUCCAAGU |
| | | AAAGCCACGAGGUCGGGGCGAGGGCAGAGGUGAUCACCUGCGUGUC |
| | | CCAUCUACAGACCUGCAGCUUCAUAAAACUUCUGAUUUCUCUUCAG |
| | | CUUUGAAAAGGGUUACCCUGGGCACUGGCCUAGAGCCUCACCUCCU |
| | | AAUAGACUUAGCCCCAUGAGUUUGCCAUGUUGAGCAGGACUAUUUC |
| | | UGGCACUUGCAAGUCCCAUGAUUUCUUCGGUAAUUCUGAGGGUGGG |
| | | GGGAGGGACAUGAAAUCAUCUUAGCUUAGCUUUCUGUCUGUGAAU |
| | | GUCUAUAUAGUGUAUUGUGUGUUUUAACAAAUGAUUUACACUGAC |
| | | UGUUGCUGUAAAAGUGAAUUUGGAAAUAAAGUUAUUACUCUGAUU |
| | | AAA |
| 27 | 12 | GCAGUCACCGCCACCCACCAGCUCCGGCACCAACAGCAGCGCCGCUG |
| | | CCACCGCCCACCUUCUGCCGCCGCCACCACAGCCACCUUCUCCUCCU |
| | | CCGCUGUCCUCUCCCGUCCUCGCCUCUGUCGACUAUCAGACAGGCUC |
| | | UGCUGAUGCUGUCCCUCUCCUGUUCAGUCGUGCCCUCACCGUUAAA |
| | | GAGAAAGAGCAAACUGCUGGGCAGCAGCAUUGAUUUUUUUAAUGA |
| | | AGUGGAAAGAGAGCUGGGAAUAACAAGUCGGGCCCACCUCACCUGC |
| | | CUCACCUGGUGAACUUUGAACCAGGAUGGCUGAGCCCCGCCAGGAG |
| | | UUCGAAGUGAUGGAAGAUCACGCUGGGACGUACGGGUUGGGGGAC |
| | | AGGAAAGAUCAGGGGGGCUACACCAUGCACCAAGACCAAGAGGGUG |
| | | ACACGGACGCUGGCCUGAAAGCUGAAGAAGCAGGCAUUGGAGACAC |
| | | CCCCAGCCUGGAAGACGAAGCUGCUGGUCACGUGACCCAAGCUCGC |
| | | AUGGUCAGUAAAAGCAAAGACGGGACUGGAAGCGAUGACAAAAAA |
| | | GCCAAGGGGGCUGAUGGUAAAACGAAGAUCGCCACACCGCGGGGAG |
| | | CAGCCCCUCCAGGCCAGAAGGGCCAGGCCAACGCCACCAGGAUUCC |
| | | AGCAAAAACCCCGCCCGCUCCAAAGACACCACCCAGCUCUGGUGAA |
| | | CCUCCAAAAUCAGGGGAUCGCAGCGGCUACAGCAGCCCCGGCUCCC |
| | | CAGGCACUCCCGGCAGCCGCUCCCGCACCCCGUCCCUUCCAACCCCA |
| | | CCCACCCGGGAGCCCAAGAAGGUGGCAGUGGUCCGUACUCCACCCA |
| | | AGUCGCCGUCUUCCGCCAAGAGCCGCCUGCAGACAGCCCCCGUGCCC |
| | | AUGCCAGACCUGAAGAAUGUCAAGUCCAAGAUCGGCUCCACUGAGA |
| | | ACCUGAAGCACCAGCCGGGAGGCGGGAAGGUGCAAAUAGUCUACAA |
| | | ACCAGUUGACCUGAGCAAGGUGACCUCCAAGUGUGGCUCAUUAGGC |
| | | AACAUCCAUCAUAAACCAGGAGGUGGCCAGGUGGAAGUAAAAUCUG |
| | | AGAAGCUUGACUUCAAGGACAGAGUCCAGUCGAAGAUUGGGUCCCU |
| | | GGACAAUAUCACCCACGUCCCUGGCGGAGGAAAUAAAAAGAUUGAA |
| | | ACCCACAAGCUGACCUUCCGCGAGAACGCCAAAGCCAAGACAGACC |
| | | ACGGGGCGGAGAUCGUGUACAAGUCGCCAGUGGUGUCUGGGGACAC |
| | | GUCUCCACGGCAUCUCAGCAAUGUCUCCUCCACCGGCAGCAUCGAC |
| | | AUGGUAGACUCGCCCCAGCUCGCCACGCUAGCUGACGAGGUGUCUG |
| | | CCUCCCUGGCCAAGCAGGGUUUGUGAUCAGGCCCCUGGGGCGGUCA |
| | | AUAAUUGUGGAGAGGAGAGAAUGAGAGAGUGUGGAAAAAAAAAGA |
| | | AUAAUGACCCGGCCCCCGCCCUCUGCCCCCAGCUGCUCCUCGCAGUU |
| | | CGGUUAAUUGGUUAAUCACUUAACCUGCUUUUGUCACUCGGCUUUG |
| | | GCUCGGGACUUCAAAAUCAGUGAUGGGAGUAAGAGCAAAUUUCAUC |
| | | UUUCCAAAUUGAUGGGUGGGCUAGUAAUAAAAUAUUUAAAAAAAA |
| | | ACAUUCAAAAACAUGGCCACAUCCAACAUUUCCUCAGGCAAUUCCU |
| | | UUUGAUUCUUUUUUCUUCCCCCUCCAUGUAGAAGAGGGAGAAGGAG |
| | | AGGCUCUGAAAGCUGCUUCUGGGGGAUUUCAAGGGACUGGGGGUGC |
| | | CAACCACCUCUGGCCCUGUUGUGGGGGUGUCACAGAGGCAGUGGCA |
| | | GCAACAAAGGAUUUGAAACUUGGUGUGUUCGUGGAGCCACAGGCAG |
| | | ACGAUGUCAACCUUGUGUGAGUGUGACGGGGGUUGGGGUGGGGCG |
| | | GGAGGCCACGGGGGAGGCCGAGGCAGGGGCUGGGCAGAGGGGAGAG |
| | | GAAGCACAAGAAGUGGGAGUGGGAGAGGAAGCCACGUGCUGGAGA |
| | | GUAGACAUCCCCCUCCUUGCCGCUGGGAGAGCCAAGGCCUAUGCCA |
| | | CCUGCAGCGUCUGAGCGGCCGCCUGUCCUUGGUGGCCGGGGGUGGG |
| | | GGCCUGCUGUGGGUCAGUGUGCCACCCUCUGCAGGGCAGCCUGUGG |
| | | GAGAAGGGACAGCGGGUAAAAAGAGAAGGCAAGCUGGCAGGAGGG |
| | | UGGCACUUCGUGGAUGACCUCCUUAGAAAAGACUGACCUUGAUGUC |
| | | UUGAGAGCGCUGGCCUCUUCCUCCCUCCCUGCAGGGUAGGGGGCCU |
| | | GAGUUGAGGGGCUUCCCUCUGCUCCACAGAAACCCUGUUUUAUUGA |
| | | GUUCUGAAGGUUGGAACUGCUGCCAUGAUUUUGGCCACUUUGCAGA |
| | | CCUGGGACUUUAGGGCUAACCAGUUCUCUUUGUAAGGACUUGUGCC |
| | | UCUUGGGAGACGUCCACCCGUUUCCAAGCCUGGGCCACUGGCAUCU |
| | | CUGGAGUGUGUGGGGGUCUGGGAGGCAGGUCCCGAGCCCCCUGUCC |
| | | UUCCCACGGCCACUGCAGUCACCCCGUCUGCGCCGCUGUGCUGUUG |
| | | UCUGCCGUGAGAGCCCAAUCACUGCCUAUACCCCUCAUCACACGUC |
| | | ACAAUGUCCCGAAUUCCCAGCCUCACCACCCCUUCUCAGUAAUGAC |
| | | CCUGGUUGGUUGCAGGAGGUACCUACUCCAUACUGAGGGUGAAAUU |
| | | AAGGGAAGGCAAAGUCCAGGCACAAGAGUGGGACCCCAGCCUCUCA |
| | | CUCUCAGUUCCACUCAUCCAACUGGGACCCUCACCACGAAUCUCAU |
| | | GAUCUGAUUCGGUUCCCUGUCUCCUCCUCCCGUCACAGAUGUGAGC |
| | | CAGGGCACUGCUCAGCUGUGACCCUAGGUGUUUCUGCCUUGUUGAC |
| | | AUGGAGAGAGCCCUUUCCCCUGAGAAGGCCUGGCCCCUUCCUGUGC |
| | | UGAGCCCACAGCAGCAGGCUGGGUGUCUUGGUUGUCAGUGGUGGCA |
| | | CCAGGAUGGAAGGGCAAGGCACCCAGGGCAGGCCCACAGUCCCGCU |
| | | GUCCCCCACUUGCACCCUAGCUUGUAGCUGCCAACCUCCCAGACAGC |
| | | CCAGCCCGCUGCUCAGCUCCACAUGCAUAGUAUCAGCCCUCCACACC |
| | | CGACAAAGGGGAACACACCCCCUUGGAAAUGGUUCUUUUCCCCCAG |
| | | UCCCAGCUGGAAGCCAUGCUGUCUGUUCUGCUGGAGCAGCUGAACA |
| | | UAUACAUAGAUGUUGCCCUGCCCUCCCCAUCUGCACCCUGUUGAGU |
| | | UGUAGUUGGAUUUGUCUGUUUAUGCUUGGAUUCACCAGAGUGACU |
| | | AUGAUAGUGAAAAGAAAAAAAAAAAAAAAAAAGGACGCAUGUAUC |
| | | UUGAAAUGCUUGUAAAGAGGUUUCUAACCCACCCUCACGAGGUGUC |
| | | UCUCACCCCCACACUGGGACUCGUGUGGCCUGUGUGGUGCCACCCU |
| | | GCUGGGGCCUCCCAAGUUUUGAAAGGCUUUCCUCAGCACCUGGGAC |
| | | CCAACAGAGACCAGCUUCUAGCAGCUAAGGAGGCCGUUCAGCUGUG |
| | | ACGAAGGCCUGAAGCACAGGAUUAGGACUGAAGCGAUGAUGUCCCC |
| | | UUCCCUACUUCCCCUUGGGGCUCCCUGUGUCAGGGCACAGACUAGG |
| | | UCUUGUGGCUGGUCUGGCUUGCGGCGCGAGGAUGGUUCUCUCUGGU |
| | | CAUAGCCCGAAGUCUCAUGGCAGUCCCAAAGGAGGCUUACAACUCC |
| | | UGCAUCACAAGAAAAAGGAAGCCACUGCCAGCUGGGGGGAUCUGCA |
| | | GCUCCCAGAAGCUCCGUGAGCCUCAGCCACCCCUCAGACUGGGUUC |
| | | CUCUCCAAGCUCGCCCUCUGGAGGGGCAGCGCAGCCUCCCACCAAG |
| | | GGCCCUGCGACCACAGCAGGGAUUGGGAUGAAUUGCCUGUCCUGGA |
| | | UCUGCUCUAGAGGCCCAAGCUGCCUGCCUGAGGAAGGAUGACUUGA |
| | | CAAGUCAGGAGACACUGUUCCCAAAGCCUUGACCAGAGCACCUCAG |
| | | CCCGCUGACCUUGCACAAACUCCAUCUGCUGCCAUGAGAAAAGGGA |
| | | AGCCGCCUUUGCAAAACAUUGCUGCCUAAAGAAACUCAGCAGCCUC |
| | | AGGCCCAAUUCUGCCACUUCUGGUUUGGGUACAGUUAAAGGCAACC |
| | | CUGAGGGACUUGGCAGUAGAAAUCCAGGGCCUCCCCUGGGGCUGGC |
| | | AGCUUCGUGUGCAGCUAGAGCUUUACCUGAAAGGAAGUCUCUGGGC |
| | | CCAGAACUCUCCACCAAGAGCCUCCCUGCCGUUCGCUGAGUCCCAGC |
| | | AAUUCUCCUAAGUUGAAGGGAUCUGAGAAGGAGAAGGAAAUGUGG |
| | | GGUAGAUUUGGUGGUGGUUAGAGAUAUGCCCCCCUCAUUACUGCCA |
| | | ACAGUUUCGGCUGCAUUUCUUCACGCACCUCGGUUCCUCUUCCUGA |
| | | AGUUCUUGUGCCCUGCUCUUCAGCACCAUGGGCCUUCUUAUACGGA |
| | | AGGCUCUGGGAUCUCCCCCUUGUGGGGCAGGCUCUUGGGGCCAGCC |
| | | UAAGAUCAUGGUUUAGGGUGAUCAGUGCUGGCAGAUAAAUUGAAA |
| | | AGGCACGCUGGCUUGUGAUCUUAAAUGAGGACAAUCCCCCCAGGGC |
| | | UGGGCACUCCUCCCCUCCCCUCACUUCUCCCACCUGCAGAGCCAGUG |
| | | UCCUUGGGUGGGCUAGAUAGGAUAUACUGUAUGCCGGCUCCUUCAA |
| | | GCUGCUGACUCACUUUAUCAAUAGUUCCAUUUAAAUUGACUUCAGU |
| | | GGUGAGACUGUAUCCUGUUUGCUAUUGCUUGUUGUGCUAUGGGGG |
| | | GAGGGGGGAGGAAUGUGUAAGAUAGUUAACAUGGGCAAAGGGAGA |
| | | UCUUGGGGUGCAGCACUUAAACUGCCUCGUAACCCUUUUCAUGAUU |
| | | UCAACCACAUUUGCUAGAGGGAGGGAGCAGCCACGGAGUUAGAGGC |
| | | CCUUGGGGUUUCUCUUUUCCACUGACAGGCUUUCCCAGGCAGCUGG |
| | | CUAGUUCAUUCCCUCCCCAGCCAGGUGCAGGCGUAGGAAUAUGGAC |
| | | AUCUGGUUGCUUUGGCCUGCUGCCCUCUUUCAGGGGUCCUAAGCCC |
| | | ACAAUCAUGCCUCCCUAAGACCUUGGCAUCCUUCCCUCUAAGCCGU |
| | | UGGCACCUCUGUGCCACCUCUCACACUGGCUCCAGACACACAGCCU |
| | | GUGCUUUUGGAGCUGAGAUCACUCGCUUCACCCUCCUCAUCUUUGU |
| | | UCUCCAAGUAAAGCCACGAGGUCGGGGCGAGGGCAGAGGUGAUCAC |
| | | CUGCGUGUCCCAUCUACAGACCUGCAGCUUCAUAAAACUUCUGAUU |
| | | UCUCUUCAGCUUUGAAAAGGGUUACCCUGGGCACUGGCCUAGAGCC |
| | | UCACCUCCUAAUAGACUUAGCCCCAUGAGUUUGCCAUGUUGAGCAG |
| | | GACUAUUUCUGGCACUUGCAAGUCCCAUGAUUUCUUCGGUAAUUCU |
| | | GAGGGUGGGGGGAGGGACAUGAAAUCAUCUUAGCUUAGCUUUCUG |
| | | UCUGUGAAUGUCUAUAUAGUGUAUUGUGUGUUUUAACAAAUGAUU |
| | | UACACUGACUGUUGCUGUAAAAGUGAAUUUGGAAAUAAAGUUAUU |
| | | ACUCUGAUUAAA |

In some embodiments, the editing of a base of the Tau mRNA results in a decreased gene translation of the Tau polypeptide. In other cases, the editing of a base of the 5′UTR of the Tau mRNA results in a decreased gene translation of the Tau polypeptide. The decreased gene translation of the Tau polypeptide can be measure by an in vitro assay. Such in vitro assay can comprise an in vitro translation assay. An in vitro translation assay can comprise a cell extract. A cell extract can comprise rabbit reticulocyte lysate, wheat germ extract, insect cells, yeast Kluyveromyces, or E. coli cell-free extract. An in vitro translation assay can comprise mixing a cell extract with a nucleic acid template, ATP, and amino acids. A nucleic acid template can comprise a mRNA template or a cDNA template. A nucleic acid template can comprise a mRNA sequence listed in TABLE 4. A nucleic acid template can comprise a cDNA sequence complementary to the mRNA sequence listed in TABLE 4. When using an in vitro translation system with a cDNA template, the cDNA can be converted to a mRNA by in vitro transcription. A cDNA can be maintained in a circular vector. A cDNA can be maintained as a linear sequence.

A list of Tau polypeptides encoded by the mRNA in TABLE 4 is listed in TABLE 5.

TABLE 5
Human MAPT Protein Isoform Sequences.

| SEQ | | |
|---|---|---|
| ID | Iso- | |
| NO | form | Peptide Sequence |
| 28 | X1 | MAEPRQEFEVMEDHAGTYGLGDRKDQGGYTMHQDQEGDTD |
| | | AGLKESPLQTPTEDGSEEPGSETSDAKSTPTAEDVTAPLV |
| | | DEGAPGKQAAAQPHTEIPEGTTAEEAGIGDTPSLEDEAAG |
| | | HVTQEELRVPGRQRKAPERPLANEISAHVQPGPCGEASGV |
| | | SGPCLGEKEPEAPVPLTASLPQHRPVCPAPPPTGGPQEPS |
| | | LEWGQKGGDWAEKGPAFPKPATTAYLHTEPESGKVVQEGF |
| | | LREPGPPGLSHQLMSGMPGAPLLPEGPREATRQPSGTGPE |
| | | DTEGGRHAPELLKHQLLGDLHQEGPPLKGAGGKERPGSKE |
| | | EVDEDRDVDESSPQDSPPSKASPAQDGRPPQTAAREATSI |
| | | PGFPAEGAIPLPVDFLSKVSTEIPASEPDGPSVGRAKGQD |
| | | APLEFTFHVEITPNVQKEQAHSEEHLGRAAFPGAPGEGPE |
| | | ARGPSLGEDTKEADLPEPSEKQPAAAPRGKPVSRVPQLKA |
| | | RMVSKSKDGTGSDDKKAKTSTRSSAKTLKNRPCLSPKHPT |
| | | PGSSDPLIQPSSPAVCPEPPSSPKYVSSVTSRTGSSGAKE |
| | | MKLKGADGKTKIATPRGAAPPGQKGQANATRIPAKTPPAP |
| | | KTPPSSGEPPKSGDRSGYSSPGSPGTPGSRSRTPSLPTPP |
| | | TREPKKVAVVRTPPKSPSSAKSRLQTAPVPMPDLKNVKSK |
| | | IGSTENLKHQPGGGKVQIINKKLDLSNVQSKCGSKDNIKH |
| | | VPGGGSVQIVYKPVDLSKVTSKCGSLGNIHHKPGGGQVEV |
| | | KSEKLDFKDRVQSKIGSLDNITHVPGGGNKKIETHKLTFR |
| | | ENAKAKTDHGAEIVYKSPVVSGDTSPRHLSNVSSTGSIDM |
| | | VDSPQLATLADEVSASLAKQGL |
| 29 | X3 | MAEPRQEFEVMEDHAGTYGLGDRKDQGGYTMHQDQEGDTD |
| | | AGLKESPLQTPTEDGSEEPGSETSDAKSTPTAEDVTAPLV |
| | | DEGAPGKQAAAQPHTEIPEGTTAEEAGIGDTPSLEDEAAG |
| | | HVTQEELRVPGRQRKAPERPLANEISAHVQPGPCGEASGV |
| | | SGPCLGEKEPEAPVPLTASLPQHRPVCPAPPPTGGPQEPS |
| | | LEWGQKGGDWAEKGPAFPKPATTAYLHTEPESGKVVQEGF |
| | | LREPGPPGLSHQLMSGMPGAPLLPEGPREATRQPSGTGPE |
| | | DTEGGRHAPELLKHQLLGDLHQEGPPLKGAGGKERPGSKE |
| | | EVDEDRDVDESSPQDSPPSKASPAQDGRPPQTAAREATSI |
| | | PGFPAEGAIPLPVDFLSKVSTEIPASEPDGPSVGRAKGQD |
| | | APLEFTFHVEITPNVQKEQAHSEEHLGRAAFPGAPGEGPE |
| | | ARGPSLGEDTKEADLPEPSEKQPAAAPRGKPVSRVPQLKA |
| | | RMVSKSKDGTGSDDKKAKTSTRSSAKTLKNRPCLSPKHPT |
| | | PGSSDPLIQPSSPAVCPEPPSSPKYVSSVTSRTGSSGAKE |
| | | MKLKGADGKTKIATPRGAAPPGQKGQANATRIPAKTPPAP |
| | | KTPPSSGEPPKSGDRSGYSSPGSPGTPGSRSRTPSLPTPP |
| | | TREPKKVAVVRTPPKSPSSAKSRLQTAPVPMPDLKNVKSK |
| | | IGSTENLKHQPGGGKVQIVYKPVDLSKVTSKCGSLGNIHH |
| | | KPGGGQVEVKSEKLDFKDRVQSKIGSLDNITHVPGGGNKK |
| | | IETHKLTFRENAKAKTDHGAEIVYKSPVVSGDTSPRHLSN |
| | | VSSTGSIDMVDSPQLATLADEVSASLAKQGL |
| 30 | X4 | MAEPRQEFEVMEDHAGTYGLGDRKDQGGYTMHQDQEGDTD |
| | | AGLKAEEAGIGDTPSLEDEAAGHVTQEELRVPGRQRKAPE |
| | | RPLANEISAHVQPGPCGEASGVSGPCLGEKEPEAPVPLTA |
| | | SLPQHRPVCPAPPPTGGPQEPSLEWGQKGGDWAEKGPAFP |
| | | KPATTAYLHTEPESGKVVQEGFLREPGPPGLSHQLMSGMP |
| | | GAPLLPEGPREATRQPSGTGPEDTEGGRHAPELLKHQLLG |
| | | DLHQEGPPLKGAGGKERPGSKEEVDEDRDVDESSPQDSPP |
| | | SKASPAQDGRPPQTAAREATSIPGFPAEGAIPLPVDFLSK |
| | | VSTEIPASEPDGPSVGRAKGQDAPLEFTFHVEITPNVQKE |
| | | QAHSEEHLGRAAFPGAPGEGPEARGPSLGEDTKEADLPEP |
| | | SEKQPAAAPRGKPVSRVPQLKARMVSKSKDGTGSDDKKAK |
| | | TSTRSSAKTLKNRPCLSPKHPTPGSSDPLIQPSSPAVCPE |
| | | PPSSPKYVSSVTSRTGSSGAKEMKLKGADGKTKIATPRGA |
| | | APPGQKGQANATRIPAKTPPAPKTPPSSGEPPKSGDRSGY |
| | | SSPGSPGTPGSRSRTPSLPTPPTREPKKVAVVRTPPKSPS |
| | | SAKSRLQTAPVPMPDLKNVKSKIGSTENLKHQPGGGKVQI |
| | | INKKLDLSNVQSKCGSKDNIKHVPGGGSVQIVYKPVDLSK |
| | | VTSKCGSLGNIHHKPGGGQVEVKSEKLDFKDRVQSKIGSL |
| | | DNITHVPGGGNKKIETHKLTFRENAKAKTDHGAEIVYKSP |
| | | VVSGDTSPRHLSNVSSTGSIDMVDSPQLATLADEVSASLA |
| | | KQGL |
| 31 | X5 | MAEPRQEFEVMEDHAGTYGLGDRKDQGGYTMHQDQEGDTD |
| | | AGLKESPLQTPTEDGSEEPGSETSDAKSTPTAEDVTAPLV |
| | | DEGAPGKQAAAQPHTEIPEGTTAEEAGIGDTPSLEDEAAG |
| | | HVTQEELRVPGRQRKAPERPLANEISAHVQPGPCGEASGV |
| | | SGPCLGEKEPEAPVPLTASLPQHRPVCPAPPPTGGPQEPS |
| | | LEWGQKGGDWAEKGPAFPKPATTAYLHTEPESGKVVQEGF |
| | | LREPGPPGLSHQLMSGMPGAPLLPEGPREATRQPSGTGPE |
| | | DTEGGRHAPELLKHQLLGDLHQEGPPLKGAGGKERPGSKE |
| | | EVDEDRDVDESSPQDSPPSKASPAQDGRPPQTAAREATSI |
| | | PGFPAEGAIPLPVDFLSKVSTEIPASEPDGPSVGRAKGQD |
| | | APLEFTFHVEITPNVQKEQAHSEEHLGRAAFPGAPGEGPE |
| | | ARGPSLGEDTKEADLPEPSEKQPAAAPRGKPVSRVPQLKA |
| | | RMVSKSKDGTGSDDKKAKGADGKTKIATPRGAAPPGQKGQ |
| | | ANATRIPAKTPPAPKTPPSSGEPPKSGDRSGYSSPGSPGT |
| | | PGSRSRTPSLPTPPTREPKKVAVVRTPPKSPSSAKSRLQT |
| | | APVPMPDLKNVKSKIGSTENLKHQPGGGKVQIINKKLDLS |
| | | NVQSKCGSKDNIKHVPGGGSVQIVYKPVDLSKVTSKCGSL |
| | | GNIHHKPGGGQVEVKSEKLDFKDRVQSKIGSLDNITHVPG |
| | | GGNKKIETHKLTFRENAKAKTDHGAEIVYKSPVVSGDTSP |
| | | RHLSNVSSTGSIDMVDSPQLATLADEVSASLAKQGL |
| 32 | X6 | MAEPRQEFEVMEDHAGTYGLGDRKDQGGYTMHQDQEGDTD |
| | | AGLKESPLQTPTEDGSEEPGSETSDAKSTPTAEDVTAPLV |
| | | DEGAPGKQAAAQPHTEIPEGTTAEEAGIGDTPSLEDEAAG |
| | | HVTQEELRVPGRQRKAPERPLANEISAHVQPGPCGEASGV |
| | | SGPCLGEKEPEAPVPLTASLPQHRPVCPAPPPTGGPQEPS |
| | | LEWGQKGGDWAEKGPAFPKPATTAYLHTEPESGKVVQEGF |
| | | LREPGPPGLSHQLMSGMPGAPLLPEGPREATRQPSGTGPE |
| | | DTEGGRHAPELLKHQLLGDLHQEGPPLKGAGGKERPGSKE |
| | | EVDEDRDVDESSPQDSPPSKASPAQDGRPPQTAAREATSI |
| | | PGFPAEGAIPLPVDFLSKVSTEIPASEPDGPSVGRAKGQD |
| | | APLEFTFHVEITPNVQKEQAHSEEHLGRAAFPGAPGEGPE |
| | | ARGPSLGEDTKEADLPEPSEKQPAAAPRGKPVSRVPQLKA |
| | | RMVSKSKDGTGSDDKKAKGADGKTKIATPRGAAPPGQKGQ |
| | | ANATRIPAKTPPAPKTPPSSGEPPKSGDRSGYSSPGSPGT |
| | | PGSRSRTPSLPTPPTREPKKVAVVRTPPKSPSSAKSRLQT |
| | | APVPMPDLKNVKSKIGSTENLKHQPGGGKVQIVYKPVDLS |
| | | KVTSKCGSLGNIHHKPGGGQVEVKSEKLDFKDRVQSKIGS |
| | | LDNITHVPGGGNKKIETHKLTFRENAKAKTDHGAEIVYKS |
| | | PVVSGDTSPRHLSNVSSTGSIDMVDSPQLATLADEVSASL |
| | | AKQGL |
| 33 | X7 | MAEPRQEFEVMEDHAGTYGLGDRKDQGGYTMHQDQEGDTD |
| | | AGLKESPLQTPTEDGSEEPGSETSDAKSTPTAEDVTAPLV |
| | | DEGAPGKQAAAQPHTEIPEGTTAEEAGIGDTPSLEDEAAG |
| | | HVTQARMVSKSKDGTGSDDKKAKTSTRSSAKTLKNRPCLS |
| | | PKHPTPGSSDPLIQPSSPAVCPEPPSSPKYVSSVTSRTGS |
| | | SGAKEMKLKGADGKTKIATPRGAAPPGQKGQANATRIPAK |
| | | TPPAPKTPPSSGEPPKSGDRSGYSSPGSPGTPGSRSRTPS |
| | | LPTPPTREPKKVAVVRTPPKSPSSAKSRLQTAPVPMPDLK |
| | | NVKSKIGSTENLKHQPGGGKVQIINKKLDLSNVQSKCGSK |
| | | DNIKHVPGGGSVQIVYKPVDLSKVTSKCGSLGNIHHKPGG |
| | | GQVEVKSEKLDFKDRVQSKIGSLDNITHVPGGGNKKIETH |
| | | KLTFRENAKAKTDHGAEIVYKSPVVSGDTSPRHLSNVSST |
| | | GSIDMVDSPQLATLADEVSASLAKQGL |
| 34 | X8 | MAEPRQEFEVMEDHAGTYGLGDRKDQGGYTMHQDQEGDTD |
| | | AGLKESPLQTPTEDGSEEPGSETSDAKSTPTAEAEEAGIG |
| | | DTPSLEDEAAGHVTQARMVSKSKDGTGSDDKKAKTSTRSS |
| | | AKTLKNRPCLSPKHPTPGSSDPLIQPSSPAVCPEPPSSPK |
| | | YVSSVTSRTGSSGAKEMKLKGADGKTKIATPRGAAPPGQK |
| | | GQANATRIPAKTPPAPKTPPSSGEPPKSGDRSGYSSPGSP |
| | | GTPGSRSRTPSLPTPPTREPKKVAVVRTPPKSPSSAKSRL |
| | | QTAPVPMPDLKNVKSKIGSTENLKHQPGGGKVQIINKKLD |
| | | LSNVQSKCGSKDNIKHVPGGGSVQIVYKPVDLSKVTSKCG |
| | | SLGNIHHKPGGGQVEVKSEKLDFKDRVQSKIGSLDNITHV |
| | | PGGGNKKIETHKLTFRENAKAKTDHGAEIVYKSPVVSGDT |
| | | SPRHLSNVSSTGSIDMVDSPQLATLADEVSASLAKQGL |
| 35 | X9 | MAEPRQEFEVMEDHAGTYGLGDRKDQGGYTMHQDQEGDTD |
| | | AGLKAEEAGIGDTPSLEDEAAGHVTQARMVSKSKDGTGSD |
| | | DKKAKTSTRSSAKTLKNRPCLSPKHPTPGSSDPLIQPSSP |
| | | AVCPEPPSSPKYVSSVTSRTGSSGAKEMKLKGADGKTKIA |
| | | TPRGAAPPGQKGQANATRIPAKTPPAPKTPPSSGEPPKSG |
| | | DRSGYSSPGSPGTPGSRSRTPSLPTPPTREPKKVAVVRTP |
| | | PKSPSSAKSRLQTAPVPMPDLKNVKSKIGSTENLKHQPGG |
| | | GKVQIINKKLDLSNVQSKCGSKDNIKHVPGGGSVQIVYKP |
| | | VDLSKVTSKCGSLGNIHHKPGGGQVEVKSEKLDFKDRVQS |
| | | KIGSLDNITHVPGGGNKKIETHKLTFRENAKAKTDHGAEI |
| | | VYKSPVVSGDTSPRHLSNVSSTGSIDMVDSPQLATLADEV |
| | | SASLAKQGL |

### Alpha-Synuclein (SNCA)

The Alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid β component (NAC) domain, and a C-terminal acidic domain. The lipid-binding domain consisting of five KXKEGV (SEQ ID NO: 194) imperfect repeats. The NAC domain consists of a GAV motif with a VGGAVVTGV (SEQ ID NO: 195) consensus sequence and three GXXX sub-motifs—where X is any of Gly, Ala, Val, Ile, Leu, Phe, Tyr, Trp, Thr, Ser or Met. The C-terminal acidic domain contains a copper-binding motif with a DPDNEA (SEQ ID NO: 196) consensus sequence. Molecularly, Alpha-synuclein is suggested to play a role in neuronal transmission and DNA repair. Complete mRNA sequences are shown in TABLE 6.

In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. In some cases, a polynucleotide comprises a targeting sequence that can hybridize to at least a portion of a sequence of TABLE 6. In some cases, a polynucleotide comprises a targeting sequence that can hybridize to at least a portion of a sequence that comprises at least about 80%, 85%, 90%, 95%, 97%, or 99% sequence identity to a sequence of TABLE 6 can be targeted. Suitable regions include but are not limited to a N-terminal A2 lipid-binding alpha-helix domain, a Non-amyloid β component (NAC) domain, or a C-terminal acidic domain.

In some aspects, an alpha-synuclein mRNA sequence is targeted. In some cases, any one of the 3,177 residues of the sequence may be targeted utilizing the compositions and method provided herein. In some cases, a target residue may be located among residues 1 to 100, from 99 to 200, from 199 to 300, from 299 to 400, from 399 to 500, from 499 to 600, from 599 to 700, from 699 to 800, from 799 to 900, from 899 to 1000, from 999 to 1100, from 1099 to 1200, from 1199 to 1300, from 1299 to 1400, from 1399 to 1500, from 1499 to 1600, from 1599 to 1700, from 1699 to 1800, from 1799 to 1900, from 1899 to 2000, from 1999 to 2100, from 2099 to 2200, from 2199 to 2300, from 2299 to 2400, from 2399 to 2500, from 2499 to 2600, from 2599 to 2700, from 2699 to 2800, from 2799 to 2900, from 2899 to 3000, from 2999 to 3100, from 3099 to 3177, or any combination thereof.

TABLE 6
Human Alpha-synuclein mRNA Isoform Sequences.
Sequences derived from NCBI SNCA sequence
corresponding to gene ID 6622; Assembly GRCh38.p13
(GCF_000001405.39); NC_000004.12
(89724099 . . . 89838324, complement).

| SEQ | | |
|---|---|---|
| ID | | |
| NO | Isoform | mRNA sequence |
| 36 | 1 | GGCGACGACCAGAAGGGGCCCAAGAGAGGGGGCGAGCGACCGAGCG |
| | | CCGCGACGCGGAAGUGAGGUGCGUGCGGGCUGCAGCGCAGACCCCG |
| | | GCCCGGCCCCUCCGAGAGCGUCCUGGGCGCUCCCUCACGCCUUGCCU |
| | | UCAAGCCUUCUGCCUUUCCACCCUCGUGAGCGGAGAACUGGGAGUG |
| | | GCCAUUCGACGACAGUGUGGUGUAAAGGAAUUCAUUAGCCAUGGAU |
| | | GUAUUCAUGAAAGGACUUUCAAAGGCCAAGGAGGGAGUUGUGGCU |
| | | GCUGCUGAGAAAACCAAACAGGGUGUGGCAGAAGCAGCAGGAAAGA |
| | | CAAAAGAGGGUGUUCUCUAUGUAGGCUCCAAAACCAAGGAGGGAGU |
| | | GGUGCAUGGUGUGGCAACAGUGGCUGAGAAGACCAAAGAGCAAGU |
| | | GACAAAUGUUGGAGGAGCAGUGGUGACGGGUGUGACAGCAGUAGC |
| | | CCAGAAGACAGUGGAGGGAGCAGGGAGCAUUGCAGCAGCCACUGGC |
| | | UUUGUCAAAAAGGACCAGUUGGGCAAGAAUGAAGAAGGAGCCCCAC |
| | | AGGAAGGAAUUCUGGAAGAUAUGCCUGUGGAUCCUGACAAUGAGG |
| | | CUUAUGAAAUGCCUUCUGAGGAAGGGUAUCAAGACUACGAACCUGA |
| | | AGCCUAAGAAAUAUCUUUGCUCCCAGUUUCUUGAGAUCUGCUGACA |
| | | GAUGUUCCAUCCUGUACAAGUGCUCAGUUCCAAUGUGCCCAGUCAU |
| | | GACAUUUCUCAAAGUUUUUACAGUGUAUCUCGAAGUCUUCCAUCAG |
| | | CAGUGAUUGAAGUAUCUGUACCUGCCCCCACUCAGCAUUUCGGUGC |
| | | UUCCCUUUCACUGAAGUGAAUACAUGGUAGCAGGGUCUUUGUGUGC |
| | | UGUGGAUUUUGUGGCUUCAAUCUACGAUGUUAAAACAAAUUAAAA |
| | | ACACCUAAGUGACUACCACUUAUUUCUAAAUCCUCACUAUUUUUUU |
| | | GUUGCUGUUGUUCAGAAGUUGUUAGUGAUUUGCUAUCAUAUAUUA |
| | | UAAGAUUUUUAGGUGUCUUUUAAUGAUACUGUCUAAGAAUAAUGA |
| | | CGUAUUGUGAAAUUUGUUAAUAUAUAUAAUACUUAAAAAUAUGUG |
| | | AGCAUGAAACUAUGCACCUAUAAAUACUAAAUAUGAAAUUUUACCA |
| | | UUUUGCGAUGUGUUUUAUUCACUUGUGUUUGUAUAUAAAUGGUGA |
| | | GAAUUAAAAUAAAACGUUAUCUCAUUGCAAAAAUAUUUUAUUUUU |
| | | AUCCCAUCUCACUUUAAUAAUAAAAAUCAUGCUUAUAAGCAACAUG |
| | | AAUUAAGAACUGACACAAAGGACAAAAAUAUAAAGUUAUUAAUAG |
| | | CCAUUUGAAGAAGGAGGAAUUUUAGAAGAGGUAGAGAAAAUGGAA |
| | | CAUUAACCCUACACUCGGAAUUCCCUGAAGCAACACUGCCAGAAGU |
| | | GUGUUUUGGUAUGCACUGGUUCCUUAAGUGGCUGUGAUUAAUUAU |
| | | UGAAAGUGGGGUGUUGAAGACCCCAACUACUAUUGUAGAGUGGUC |
| | | UAUUUCUCCCUUCAAUCCUGUCAAUGUUUGCUUUACGUAUUUUGGG |
| | | GAACUGUUGUUUGAUGUGUAUGUGUUUAUAAUUGUUAUACAUUUU |
| | | UAAUUGAGCCUUUUAUUAACAUAUAUUGUUAUUUUUGUCUCGAAA |
| | | UAAUUUUUUAGUUAAAAUCUAUUUUGUCUGAUAUUGGUGUGAAUG |
| | | CUGUACCUUUCUGACAAUAAAUAAUAUUCGACCAUGAAUAAAAAAA |
| | | AAAAAAAAGUGGGUUCCCGGGAACUAAGCAGUGUAGAAGAUGAUU |
| | | UUGACUACACCCUCCUUAGAGAGCCAUAAGACACAUUAGCACAUAU |
| | | UAGCACAUUCAAGGCUCUGAGAGAAUGUGGUUAACUUUGUUUAAC |
| | | UCAGCAUUCCUCACUUUUUUUUUUUAAUCAUCAGAAAUUCUCUCUC |
| | | UCUCUCUCUCUUUUUCUCUCGCUCUCUUUUUUUUUUUUUUUUUACA |
| | | GGAAAUGCCUUUAAACAUCGUUGGAACUACCAGAGUCACCUUAAAG |
| | | GAGAUCAAUUCUCUAGACUGAUAAAAAUUUCAUGGCCUCCUUUAAA |
| | | UGUUGCCAAAUAUAUGAAUUCUAGGAUUUUUCCUUAGGAAAGGUU |
| | | UUUCUCUUUCAGGGAAGAUCUAUUAACUCCCCAUGGGUGCUGAAAA |
| | | UAAACUUGAUGGUGAAAAACUCUGUAUAAAUUAAUUUAAAAAUUA |
| | | UUUGGUUUCUCUUUUUAAUUAUUCUGGGGCAUAGUCAUUUCUAAA |
| | | AGUCACUAGUAGAAAGUAUAAUUUCAAGACAGAAUAUUCUAGACA |
| | | UGCUAGCAGUUUAUAUGUAUUCAUGAGUAAUGUGAUAUAUAUUGG |
| | | GCGCUGGUGAGGAAGGAAGGAGGAAUGAGUGACUAUAAGGAUGGU |
| | | UACCAUAGAAACUUCCUUUUUUACCUAAUUGAAGAGAGACUACUAC |
| | | AGAGUGCUAAGCUGCAUGUGUCAUCUUACACUAGAGAGAAAUGGU |
| | | AAGUUUCUUGUUUUAUUUAAGUUAUGUUUAAGCAAGGAAAGGAUU |
| | | UGUUAUUGAACAGUAUAUUUCAGGAAGGUUAGAAAGUGGCGGUUA |
| | | GGAUAUAUUUUAAAUCUACCUAAAGCAGCAUAUUUUAAAAAUUUA |
| | | AAAGUAUUGGUAUUAAAUUAAGAAAUAGAGGACAGAACUAGACUG |
| | | AUAGCAGUGACCUAGAACAAUUUGAGAUUAGGAAAGUUGUGACCA |
| | | UGAAUUUAAGGAUUUAUGUGGAUACAAAUUCUCCUUUAAAGUGUU |
| | | UCUUCCCUUAAUAUUUAUCUGACGGUAAUUUUUGAGCAGUGAAUU |
| | | ACUUUAUAUAUCUUAAUAGUUUAUUUGGGACCAAACACUUAAACA |
| | | AAAAGUUCUUUAAGUCAUAUAAGCCUUUUCAGGAAGCUUGUCUCAU |
| | | AUUCACUCCCGAGACAUUCACCUGCCAAGUGGCCUGAGGAUCAAUC |
| | | CAGUCCUAGGUUUAUUUUGCAGACUUACAUUCUCCCAAGUUAUUCA |
| | | GCCUCAUAUGACUCCACGGUCGGCUUUACCAAAACAGUUCAGAGUG |
| | | CACUUUGGCACACAAUUGGGAACAGAACAAUCUAAUGUGUGGUUUG |
| | | GUAUUCCAAGUGGGGUCUUUUUCAGAAUCUCUGCACUAGUGUGAGA |
| | | UGCAAACAUGUUUCCUCAUCUUUCUGGCUUAUCCAGUAUGUAGCUA |
| | | UUUGUGACAUAAUAAAUAUAUACAUAUAUGAAAAUA |
| 37 | 2 | GGCGACGACCAGAAGGGGCCCAAGAGAGGGGGCGAGCGACCGAGCG |
| | | CCGCGACGCGGAAGUGAGGUGCGUGCGGGCUGCAGCGCAGACCCCG |
| | | GCCCGGCCCCUCCGAGAGCGUCCUGGGCGCUCCCUCACGCCUUGCCU |
| | | UCAAGCCUUCUGCCUUUCCACCCUCGUGAGCGGAGAACUGGGAGUG |
| | | GCCAUUCGACGACAGGUUAGCGGGUUUGCCUCCCACUCCCCCAGCC |
| | | UCGCGUCGCCGGCUCACAGCGGCCUCCUCUGGGGACAGUCCCCCCCG |
| | | GGUGCCGCCUCCGCCCUUCCUGUGCGCUCCUUUUCCUUCUUCUUUCC |
| | | UAUUAAAUAUUAUUUGGGAAUUGUUUAAAUUUUUUUUUUAAAAAA |
| | | AGAGAGAGGCGGGGAGGAGUCGGAGUUGUGGAGAAGCAGAGGGAC |
| | | UCAGUGUGGUGUAAAGGAAUUCAUUAGCCAUGGAUGUAUUCAUGA |
| | | AAGGACUUUCAAAGGCCAAGGAGGGAGUUGUGGCUGCUGCUGAGA |
| | | AAACCAAACAGGGUGUGGCAGAAGCAGCAGGAAAGACAAAAGAGG |
| | | GUGUUCUCUAUGUAGGCUCCAAAACCAAGGAGGGAGUGGUGCAUGG |
| | | UGUGGCAACAGUGGCUGAGAAGACCAAAGAGCAAGUGACAAAUGU |
| | | UGGAGGAGCAGUGGUGACGGGUGUGACAGCAGUAGCCCAGAAGACA |
| | | GUGGAGGGAGCAGGGAGCAUUGCAGCAGCCACUGGCUUUGUCAAAA |
| | | AGGACCAGUUGGGCAAGAAUGAAGAAGGAGCCCCACAGGAAGGAAU |
| | | UCUGGAAGAUAUGCCUGUGGAUCCUGACAAUGAGGCUUAUGAAAU |
| | | GCCUUCUGAGGAAGGGUAUCAAGACUACGAACCUGAAGCCUAAGAA |
| | | AUAUCUUUGCUCCCAGUUUCUUGAGAUCUGCUGACAGAUGUUCCAU |
| | | CCUGUACAAGUGCUCAGUUCCAAUGUGCCCAGUCAUGACAUUUCUC |
| | | AAAGUUUUUACAGUGUAUCUCGAAGUCUUCCAUCAGCAGUGAUUGA |
| | | AGUAUCUGUACCUGCCCCCACUCAGCAUUUCGGUGCUUCCCUUUCA |
| | | CUGAAGUGAAUACAUGGUAGCAGGGUCUUUGUGUGCUGUGGAUUU |
| | | UGUGGCUUCAAUCUACGAUGUUAAAACAAAUUAAAAACACCUAAGU |
| | | GACUACCACUUAUUUCUAAAUCCUCACUAUUUUUUUGUUGCUGUUG |
| | | UUCAGAAGUUGUUAGUGAUUUGCUAUCAUAUAUUAUAAGAUUUUU |
| | | AGGUGUCUUUUAAUGAUACUGUCUAAGAAUAAUGACGUAUUGUGA |
| | | AAUUUGUUAAUAUAUAUAAUACUUAAAAAUAUGUGAGCAUGAAAC |
| | | UAUGCACCUAUAAAUACUAAAUAUGAAAUUUUACCAUUUUGCGAU |
| | | GUGUUUUAUUCACUUGUGUUUGUAUAUAAAUGGUGAGAAUUAAAA |
| | | UAAAACGUUAUCUCAUUGCAAAAAUAUUUUAUUUUUAUCCCAUCUC |
| | | ACUUUAAUAAUAAAAAUCAUGCUUAUAAGCAACAUGAAUUAAGAA |
| | | CUGACACAAAGGACAAAAAUAUAAAGUUAUUAAUAGCCAUUUGAA |
| | | GAAGGAGGAAUUUUAGAAGAGGUAGAGAAAAUGGAACAUUAACCC |
| | | UACACUCGGAAUUCCCUGAAGCAACACUGCCAGAAGUGUGUUUUGG |
| | | UAUGCACUGGUUCCUUAAGUGGCUGUGAUUAAUUAUUGAAAGUGG |
| | | GGUGUUGAAGACCCCAACUACUAUUGUAGAGUGGUCUAUUUCUCCC |
| | | UUCAAUCCUGUCAAUGUUUGCUUUACGUAUUUUGGGGAACUGUUG |
| | | UUUGAUGUGUAUGUGUUUAUAAUUGUUAUACAUUUUUAAUUGAGC |
| | | CUUUUAUUAACAUAUAUUGUUAUUUUUGUCUCGAAAUAAUUUUUU |
| | | AGUUAAAAUCUAUUUUGUCUGAUAUUGGUGUGAAUGCUGUACCUU |
| | | UCUGACAAUAAAUAAUAUUCGACCAUGAAUAAAAAAAAAAAAAAA |
| | | GUGGGUUCCCGGGAACUAAGCAGUGUAGAAGAUGAUUUUGACUAC |
| | | ACCCUCCUUAGAGAGCCAUAAGACACAUUAGCACAUAUUAGCACAU |
| | | UCAAGGCUCUGAGAGAAUGUGGUUAACUUUGUUUAACUCAGCAUUC |
| | | CUCACUUUUUUUUUUUAAUCAUCAGAAAUUCUCUCUCUCUCUCUCU |
| | | CUUUUUCUCUCGCUCUCUUUUUUUUUUUUUUUUUACAGGAAAUGCC |
| | | UUUAAACAUCGUUGGAACUACCAGAGUCACCUUAAAGGAGAUCAAU |
| | | UCUCUAGACUGAUAAAAAUUUCAUGGCCUCCUUUAAAUGUUGCCAA |
| | | AUAUAUGAAUUCUAGGAUUUUUCCUUAGGAAAGGUUUUUCUCUUU |
| | | CAGGGAAGAUCUAUUAACUCCCCAUGGGUGCUGAAAAUAAACUUGA |
| | | UGGUGAAAAACUCUGUAUAAAUUAAUUUAAAAAUUAUUUGGUUUC |
| | | UCUUUUUAAUUAUUCUGGGGCAUAGUCAUUUCUAAAAGUCACUAG |
| | | UAGAAAGUAUAAUUUCAAGACAGAAUAUUCUAGACAUGCUAGCAG |
| | | UUUAUAUGUAUUCAUGAGUAAUGUGAUAUAUAUUGGGCGCUGGUG |
| | | AGGAAGGAAGGAGGAAUGAGUGACUAUAAGGAUGGUUACCAUAGA |
| | | AACUUCCUUUUUUACCUAAUUGAAGAGAGACUACUACAGAGUGCUA |
| | | AGCUGCAUGUGUCAUCUUACACUAGAGAGAAAUGGUAAGUUUCUU |
| | | GUUUUAUUUAAGUUAUGUUUAAGCAAGGAAAGGAUUUGUUAUUGA |
| | | ACAGUAUAUUUCAGGAAGGUUAGAAAGUGGCGGUUAGGAUAUAUU |
| | | UUAAAUCUACCUAAAGCAGCAUAUUUUAAAAAUUUAAAAGUAUUG |
| | | GUAUUAAAUUAAGAAAUAGAGGACAGAACUAGACUGAUAGCAGUG |
| | | ACCUAGAACAAUUUGAGAUUAGGAAAGUUGUGACCAUGAAUUUAA |
| | | GGAUUUAUGUGGAUACAAAUUCUCCUUUAAAGUGUUUCUUCCCUUA |
| | | AUAUUUAUCUGACGGUAAUUUUUGAGCAGUGAAUUACUUUAUAUA |
| | | UCUUAAUAGUUUAUUUGGGACCAAACACUUAAACAAAAAGUUCUU |
| | | UAAGUCAUAUAAGCCUUUUCAGGAAGCUUGUCUCAUAUUCACUCCC |
| | | GAGACAUUCACCUGCCAAGUGGCCUGAGGAUCAAUCCAGUCCUAGG |
| | | UUUAUUUUGCAGACUUACAUUCUCCCAAGUUAUUCAGCCUCAUAUG |
| | | ACUCCACGGUCGGCUUUACCAAAACAGUUCAGAGUGCACUUUGGCA |
| | | CACAAUUGGGAACAGAACAAUCUAAUGUGUGGUUUGGUAUUCCAA |
| | | GUGGGGUCUUUUUCAGAAUCUCUGCACUAGUGUGAGAUGCAAACAU |
| | | GUUUCCUCAUCUUUCUGGCUUAUCCAGUAUGUAGCUAUUUGUGACA |
| | | UAAUAAAUAUAUACAUAUAUGAAAAUA |
| 38 | 3 | GCUUCUCCAUUCUGGUGUGAUCCAGGAACAGCUGUCUUCCAGCUCU |
| | | GAAAGAGUGUGGUGUAAAGGAAUUCAUUAGCCAUGGAUGUAUUCA |
| | | UGAAAGGACUUUCAAAGGCCAAGGAGGGAGUUGUGGCUGCUGCUG |
| | | AGAAAACCAAACAGGGUGUGGCAGAAGCAGCAGGAAAGACAAAAG |
| | | AGGGUGUUCUCUAUGUAGGCUCCAAAACCAAGGAGGGAGUGGUGCA |
| | | UGGUGUGGCAACAGUGGCUGAGAAGACCAAAGAGCAAGUGACAAA |
| | | UGUUGGAGGAGCAGUGGUGACGGGUGUGACAGCAGUAGCCCAGAA |
| | | GACAGUGGAGGGAGCAGGGAGCAUUGCAGCAGCCACUGGCUUUGUC |
| | | AAAAAGGACCAGUUGGGCAAGAAUGAAGAAGGAGCCCCACAGGAAG |
| | | GAAUUCUGGAAGAUAUGCCUGUGGAUCCUGACAAUGAGGCUUAUG |
| | | AAAUGCCUUCUGAGGAAGGGUAUCAAGACUACGAACCUGAAGCCUA |
| | | AGAAAUAUCUUUGCUCCCAGUUUCUUGAGAUCUGCUGACAGAUGUU |
| | | CCAUCCUGUACAAGUGCUCAGUUCCAAUGUGCCCAGUCAUGACAUU |
| | | UCUCAAAGUUUUUACAGUGUAUCUCGAAGUCUUCCAUCAGCAGUGA |
| | | UUGAAGUAUCUGUACCUGCCCCCACUCAGCAUUUCGGUGCUUCCCU |
| | | UUCACUGAAGUGAAUACAUGGUAGCAGGGUCUUUGUGUGCUGUGG |
| | | AUUUUGUGGCUUCAAUCUACGAUGUUAAAACAAAUUAAAAACACCU |
| | | AAGUGACUACCACUUAUUUCUAAAUCCUCACUAUUUUUUUGUUGCU |
| | | GUUGUUCAGAAGUUGUUAGUGAUUUGCUAUCAUAUAUUAUAAGAU |
| | | UUUUAGGUGUCUUUUAAUGAUACUGUCUAAGAAUAAUGACGUAUU |
| | | GUGAAAUUUGUUAAUAUAUAUAAUACUUAAAAAUAUGUGAGCAUG |
| | | AAACUAUGCACCUAUAAAUACUAAAUAUGAAAUUUUACCAUUUUGC |
| | | GAUGUGUUUUAUUCACUUGUGUUUGUAUAUAAAUGGUGAGAAUUA |
| | | AAAUAAAACGUUAUCUCAUUGCAAAAAUAUUUUAUUUUUAUCCCA |
| | | UCUCACUUUAAUAAUAAAAAUCAUGCUUAUAAGCAACAUGAAUUA |
| | | AGAACUGACACAAAGGACAAAAAUAUAAAGUUAUUAAUAGCCAUU |
| | | UGAAGAAGGAGGAAUUUUAGAAGAGGUAGAGAAAAUGGAACAUUA |
| | | ACCCUACACUCGGAAUUCCCUGAAGCAACACUGCCAGAAGUGUGUU |
| | | UUGGUAUGCACUGGUUCCUUAAGUGGCUGUGAUUAAUUAUUGAAA |
| | | GUGGGGUGUUGAAGACCCCAACUACUAUUGUAGAGUGGUCUAUUUC |
| | | UCCCUUCAAUCCUGUCAAUGUUUGCUUUACGUAUUUUGGGGAACUG |
| | | UUGUUUGAUGUGUAUGUGUUUAUAAUUGUUAUACAUUUUUAAUUG |
| | | AGCCUUUUAUUAACAUAUAUUGUUAUUUUUGUCUCGAAAUAAUUU |
| | | UUUAGUUAAAAUCUAUUUUGUCUGAUAUUGGUGUGAAUGCUGUAC |
| | | CUUUCUGACAAUAAAUAAUAUUCGACCAUGAAUAAAAAAAAAAAA |
| | | AAAGUGGGUUCCCGGGAACUAAGCAGUGUAGAAGAUGAUUUUGAC |
| | | UACACCCUCCUUAGAGAGCCAUAAGACACAUUAGCACAUAUUAGCA |
| | | CAUUCAAGGCUCUGAGAGAAUGUGGUUAACUUUGUUUAACUCAGCA |
| | | UUCCUCACUUUUUUUUUUUAAUCAUCAGAAAUUCUCUCUCUCUCUC |
| | | UCUCUUUUUCUCUCGCUCUCUUUUUUUUUUUUUUUUUACAGGAAAU |
| | | GCCUUUAAACAUCGUUGGAACUACCAGAGUCACCUUAAAGGAGAUC |
| | | AAUUCUCUAGACUGAUAAAAAUUUCAUGGCCUCCUUUAAAUGUUGC |
| | | CAAAUAUAUGAAUUCUAGGAUUUUUCCUUAGGAAAGGUUUUUCUC |
| | | UUUCAGGGAAGAUCUAUUAACUCCCCAUGGGUGCUGAAAAUAAACU |
| | | UGAUGGUGAAAAACUCUGUAUAAAUUAAUUUAAAAAUUAUUUGGU |
| | | UUCUCUUUUUAAUUAUUCUGGGGCAUAGUCAUUUCUAAAAGUCACU |
| | | AGUAGAAAGUAUAAUUUCAAGACAGAAUAUUCUAGACAUGCUAGC |
| | | AGUUUAUAUGUAUUCAUGAGUAAUGUGAUAUAUAUUGGGCGCUGG |
| | | UGAGGAAGGAAGGAGGAAUGAGUGACUAUAAGGAUGGUUACCAUA |
| | | GAAACUUCCUUUUUUACCUAAUUGAAGAGAGACUACUACAGAGUGC |
| | | UAAGCUGCAUGUGUCAUCUUACACUAGAGAGAAAUGGUAAGUUUC |
| | | UUGUUUUAUUUAAGUUAUGUUUAAGCAAGGAAAGGAUUUGUUAUU |
| | | GAACAGUAUAUUUCAGGAAGGUUAGAAAGUGGCGGUUAGGAUAUA |
| | | UUUUAAAUCUACCUAAAGCAGCAUAUUUUAAAAAUUUAAAAGUAU |
| | | UGGUAUUAAAUUAAGAAAUAGAGGACAGAACUAGACUGAUAGCAG |
| | | UGACCUAGAACAAUUUGAGAUUAGGAAAGUUGUGACCAUGAAUUU |
| | | AAGGAUUUAUGUGGAUACAAAUUCUCCUUUAAAGUGUUUCUUCCCU |
| | | UAAUAUUUAUCUGACGGUAAUUUUUGAGCAGUGAAUUACUUUAUA |
| | | UAUCUUAAUAGUUUAUUUGGGACCAAACACUUAAACAAAAAGUUC |
| | | UUUAAGUCAUAUAAGCCUUUUCAGGAAGCUUGUCUCAUAUUCACUC |
| | | CCGAGACAUUCACCUGCCAAGUGGCCUGAGGAUCAAUCCAGUCCUA |
| | | GGUUUAUUUUGCAGACUUACAUUCUCCCAAGUUAUUCAGCCUCAUA |
| | | UGACUCCACGGUCGGCUUUACCAAAACAGUUCAGAGUGCACUUUGG |
| | | CACACAAUUGGGAACAGAACAAUCUAAUGUGUGGUUUGGUAUUCCA |
| | | AGUGGGGUCUUUUUCAGAAUCUCUGCACUAGUGUGAGAUGCAAACA |
| | | UGUUUCCUCAUCUUUCUGGCUUAUCCAGUAUGUAGCUAUUUGUGAC |
| | | AUAAUAAAUAUAUACAUAUAUGAAAAUA |
| 39 | 4 | GGCGACGACCAGAAGGGGCCCAAGAGAGGGGGCGAGCGACCGAGCG |
| | | CCGCGACGCGGAAGUGAGGUGCGUGCGGGCUGCAGCGCAGACCCCG |
| | | GCCCGGCCCCUCCGAGAGCGUCCUGGGCGCUCCCUCACGCCUUGCCU |
| | | UCAAGCCUUCUGCCUUUCCACCCUCGUGAGCGGAGAACUGGGAGUG |
| | | GCCAUUCGACGACAGGUUAGCGGGUUUGCCUCCCACUCCCCCAGCC |
| | | UCGCGUCGCCGGCUCACAGCGGCCUCCUCUGGGGACAGUCCCCCCCG |
| | | GGUGCCGCCUCCGCCCUUCCUGUGCGCUCCUUUUCCUUCUUCUUUCC |
| | | UAUUAAAUAUUAUUUGGGAAUUGUUUAAAUUUUUUUUUUAAAAAA |
| | | AGAGAGAGGCGGGGAGGAGUCGGAGUUGUGGAGAAGCAGAGGGAC |
| | | UCAGUGUGGUGUAAAGGAAUUCAUUAGCCAUGGAUGUAUUCAUGA |
| | | AAGGACUUUCAAAGGCCAAGGAGGGAGUUGUGGCUGCUGCUGAGA |
| | | AAACCAAACAGGGUGUGGCAGAAGCAGCAGGAAAGACAAAAGAGG |
| | | GUGUUCUCUAUGUAGGCUCCAAAACCAAGGAGGGAGUGGUGCAUGG |
| | | UGUGGCAACAGUGGCUGAGAAGACCAAAGAGCAAGUGACAAAUGU |
| | | UGGAGGAGCAGUGGUGACGGGUGUGACAGCAGUAGCCCAGAAGACA |
| | | GUGGAGGGAGCAGGGAGCAUUGCAGCAGCCACUGGCUUUGUCAAAA |
| | | AGGACCAGUUGGGCAAGGAAGGGUAUCAAGACUACGAACCUGAAGC |
| | | CUAAGAAAUAUCUUUGCUCCCAGUUUCUUGAGAUCUGCUGACAGAU |
| | | GUUCCAUCCUGUACAAGUGCUCAGUUCCAAUGUGCCCAGUCAUGAC |
| | | AUUUCUCAAAGUUUUUACAGUGUAUCUCGAAGUCUUCCAUCAGCAG |
| | | UGAUUGAAGUAUCUGUACCUGCCCCCACUCAGCAUUUCGGUGCUUC |
| | | CCUUUCACUGAAGUGAAUACAUGGUAGCAGGGUCUUUGUGUGCUGU |
| | | GGAUUUUGUGGCUUCAAUCUACGAUGUUAAAACAAAUUAAAAACA |
| | | CCUAAGUGACUACCACUUAUUUCUAAAUCCUCACUAUUUUUUUGUU |
| | | GCUGUUGUUCAGAAGUUGUUAGUGAUUUGCUAUCAUAUAUUAUAA |
| | | GAUUUUUAGGUGUCUUUUAAUGAUACUGUCUAAGAAUAAUGACGU |
| | | AUUGUGAAAUUUGUUAAUAUAUAUAAUACUUAAAAAUAUGUGAGC |
| | | AUGAAACUAUGCACCUAUAAAUACUAAAUAUGAAAUUUUACCAUU |
| | | UUGCGAUGUGUUUUAUUCACUUGUGUUUGUAUAUAAAUGGUGAGA |
| | | AUUAAAAUAAAACGUUAUCUCAUUGCAAAAAUAUUUUAUUUUUAU |
| | | CCCAUCUCACUUUAAUAAUAAAAAUCAUGCUUAUAAGCAACAUGAA |
| | | UUAAGAACUGACACAAAGGACAAAAAUAUAAAGUUAUUAAUAGCC |
| | | AUUUGAAGAAGGAGGAAUUUUAGAAGAGGUAGAGAAAAUGGAACA |
| | | UUAACCCUACACUCGGAAUUCCCUGAAGCAACACUGCCAGAAGUGU |
| | | GUUUUGGUAUGCACUGGUUCCUUAAGUGGCUGUGAUUAAUUAUUG |
| | | AAAGUGGGGUGUUGAAGACCCCAACUACUAUUGUAGAGUGGUCUA |
| | | UUUCUCCCUUCAAUCCUGUCAAUGUUUGCUUUACGUAUUUUGGGGA |
| | | ACUGUUGUUUGAUGUGUAUGUGUUUAUAAUUGUUAUACAUUUUUA |
| | | AUUGAGCCUUUUAUUAACAUAUAUUGUUAUUUUUGUCUCGAAAUA |
| | | AUUUUUUAGUUAAAAUCUAUUUUGUCUGAUAUUGGUGUGAAUGCU |
| | | GUACCUUUCUGACAAUAAAUAAUAUUCGACCAUGAAUAAAAAAAA |
| | | AAAAAAAGUGGGUUCCCGGGAACUAAGCAGUGUAGAAGAUGAUUU |
| | | UGACUACACCCUCCUUAGAGAGCCAUAAGACACAUUAGCACAUAUU |
| | | AGCACAUUCAAGGCUCUGAGAGAAUGUGGUUAACUUUGUUUAACUC |
| | | AGCAUUCCUCACUUUUUUUUUUUAAUCAUCAGAAAUUCUCUCUCUC |
| | | UCUCUCUCUUUUUCUCUCGCUCUCUUUUUUUUUUUUUUUUUACAGG |
| | | AAAUGCCUUUAAACAUCGUUGGAACUACCAGAGUCACCUUAAAGGA |
| | | GAUCAAUUCUCUAGACUGAUAAAAAUUUCAUGGCCUCCUUUAAAUG |
| | | UUGCCAAAUAUAUGAAUUCUAGGAUUUUUCCUUAGGAAAGGUUUU |
| | | UCUCUUUCAGGGAAGAUCUAUUAACUCCCCAUGGGUGCUGAAAAUA |
| | | AACUUGAUGGUGAAAAACUCUGUAUAAAUUAAUUUAAAAAUUAUU |
| | | UGGUUUCUCUUUUUAAUUAUUCUGGGGCAUAGUCAUUUCUAAAAG |
| | | UCACUAGUAGAAAGUAUAAUUUCAAGACAGAAUAUUCUAGACAUG |
| | | CUAGCAGUUUAUAUGUAUUCAUGAGUAAUGUGAUAUAUAUUGGGC |
| | | GCUGGUGAGGAAGGAAGGAGGAAUGAGUGACUAUAAGGAUGGUUA |
| | | CCAUAGAAACUUCCUUUUUUACCUAAUUGAAGAGAGACUACUACAG |
| | | AGUGCUAAGCUGCAUGUGUCAUCUUACACUAGAGAGAAAUGGUAA |
| | | GUUUCUUGUUUUAUUUAAGUUAUGUUUAAGCAAGGAAAGGAUUUG |
| | | UUAUUGAACAGUAUAUUUCAGGAAGGUUAGAAAGUGGCGGUUAGG |
| | | AUAUAUUUUAAAUCUACCUAAAGCAGCAUAUUUUAAAAAUUUAAA |
| | | AGUAUUGGUAUUAAAUUAAGAAAUAGAGGACAGAACUAGACUGAU |
| | | AGCAGUGACCUAGAACAAUUUGAGAUUAGGAAAGUUGUGACCAUG |
| | | AAUUUAAGGAUUUAUGUGGAUACAAAUUCUCCUUUAAAGUGUUUC |
| | | UUCCCUUAAUAUUUAUCUGACGGUAAUUUUUGAGCAGUGAAUUAC |
| | | UUUAUAUAUCUUAAUAGUUUAUUUGGGACCAAACACUUAAACAAA |
| | | AAGUUCUUUAAGUCAUAUAAGCCUUUUCAGGAAGCUUGUCUCAUAU |
| | | UCACUCCCGAGACAUUCACCUGCCAAGUGGCCUGAGGAUCAAUCCA |
| | | GUCCUAGGUUUAUUUUGCAGACUUACAUUCUCCCAAGUUAUUCAGC |
| | | CUCAUAUGACUCCACGGUCGGCUUUACCAAAACAGUUCAGAGUGCA |
| | | CUUUGGCACACAAUUGGGAACAGAACAAUCUAAUGUGUGGUUUGG |
| | | UAUUCCAAGUGGGGUCUUUUUCAGAAUCUCUGCACUAGUGUGAGAU |
| | | GCAAACAUGUUUCCUCAUCUUUCUGGCUUAUCCAGUAUGUAGCUAU |
| | | UUGUGACAUAAUAAAUAUAUACAUAUAUGAAAAUA |
| 40 | 5 | GCUUCUCCAUUCUGGUGUGAUCCAGGAACAGCUGUCUUCCAGCUCU |
| | | GAAAGAGGGCUGAGAGAUUAGGCUGCUUCUCCGGGAUCCGCUUUUC |
| | | CCCGGGAAACGCGAGGAUGCUCCAUGGAGCUGUGGUGUAAAGGAAU |
| | | UCAUUAGCCAUGGAUGUAUUCAUGAAAGGACUUUCAAAGGCCAAGG |
| | | AGGGAGUUGUGGCUGCUGCUGAGAAAACCAAACAGGGUGUGGCAG |
| | | AAGCAGCAGGAAAGACAAAAGAGGGUGUUCUCUAUGUAGGCUCCAA |
| | | AACCAAGGAGGGAGUGGUGCAUGGUGUGGCAACAGUGGCUGAGAA |
| | | GACCAAAGAGCAAGUGACAAAUGUUGGAGGAGCAGUGGUGACGGG |
| | | UGUGACAGCAGUAGCCCAGAAGACAGUGGAGGGAGCAGGGAGCAUU |
| | | GCAGCAGCCACUGGCUUUGUCAAAAAGGACCAGUUGGGCAAGAAUG |
| | | AAGAAGGAGCCCCACAGGAAGGAAUUCUGGAAGAUAUGCCUGUGGA |
| | | UCCUGACAAUGAGGCUUAUGAAAUGCCUUCUGAGGAAGGGUAUCAA |
| | | GACUACGAACCUGAAGCCUAAGAAAUAUCUUUGCUCCCAGUUUCUU |
| | | GAGAUCUGCUGACAGAUGUUCCAUCCUGUACAAGUGCUCAGUUCCA |
| | | AUGUGCCCAGUCAUGACAUUUCUCAAAGUUUUUACAGUGUAUCUCG |
| | | AAGUCUUCCAUCAGCAGUGAUUGAAGUAUCUGUACCUGCCCCCACU |
| | | CAGCAUUUCGGUGCUUCCCUUUCACUGAAGUGAAUACAUGGUAGCA |
| | | GGGUCUUUGUGUGCUGUGGAUUUUGUGGCUUCAAUCUACGAUGUU |
| | | AAAACAAAUUAAAAACACCUAAGUGACUACCACUUAUUUCUAAAUC |
| | | CUCACUAUUUUUUUGUUGCUGUUGUUCAGAAGUUGUUAGUGAUUU |
| | | GCUAUCAUAUAUUAUAAGAUUUUUAGGUGUCUUUUAAUGAUACUG |
| | | UCUAAGAAUAAUGACGUAUUGUGAAAUUUGUUAAUAUAUAUAAUA |
| | | CUUAAAAAUAUGUGAGCAUGAAACUAUGCACCUAUAAAUACUAAA |
| | | UAUGAAAUUUUACCAUUUUGCGAUGUGUUUUAUUCACUUGUGUUU |
| | | GUAUAUAAAUGGUGAGAAUUAAAAUAAAACGUUAUCUCAUUGCAA |
| | | AAAUAUUUUAUUUUUAUCCCAUCUCACUUUAAUAAUAAAAAUCAU |
| | | GCUUAUAAGCAACAUGAAUUAAGAACUGACACAAAGGACAAAAAU |
| | | AUAAAGUUAUUAAUAGCCAUUUGAAGAAGGAGGAAUUUUAGAAGA |
| | | GGUAGAGAAAAUGGAACAUUAACCCUACACUCGGAAUUCCCUGAAG |
| | | CAACACUGCCAGAAGUGUGUUUUGGUAUGCACUGGUUCCUUAAGUG |
| | | GCUGUGAUUAAUUAUUGAAAGUGGGGUGUUGAAGACCCCAACUAC |
| | | UAUUGUAGAGUGGUCUAUUUCUCCCUUCAAUCCUGUCAAUGUUUGC |
| | | UUUACGUAUUUUGGGGAACUGUUGUUUGAUGUGUAUGUGUUUAUA |
| | | AUUGUUAUACAUUUUUAAUUGAGCCUUUUAUUAACAUAUAUUGUU |
| | | AUUUUUGUCUCGAAAUAAUUUUUUAGUUAAAAUCUAUUUUGUCUG |
| | | AUAUUGGUGUGAAUGCUGUACCUUUCUGACAAUAAAUAAUAUUCG |
| | | ACCAUGAAUAAAAAAAAAAAAAAAGUGGGUUCCCGGGAACUAAGC |
| | | AGUGUAGAAGAUGAUUUUGACUACACCCUCCUUAGAGAGCCAUAAG |
| | | ACACAUUAGCACAUAUUAGCACAUUCAAGGCUCUGAGAGAAUGUGG |
| | | UUAACUUUGUUUAACUCAGCAUUCCUCACUUUUUUUUUUUAAUCAU |
| | | CAGAAAUUCUCUCUCUCUCUCUCUCUUUUUCUCUCGCUCUCUUUUU |
| | | UUUUUUUUUUUUACAGGAAAUGCCUUUAAACAUCGUUGGAACUACC |
| | | AGAGUCACCUUAAAGGAGAUCAAUUCUCUAGACUGAUAAAAAUUUC |
| | | AUGGCCUCCUUUAAAUGUUGCCAAAUAUAUGAAUUCUAGGAUUUU |
| | | UCCUUAGGAAAGGUUUUUCUCUUUCAGGGAAGAUCUAUUAACUCCC |
| | | CAUGGGUGCUGAAAAUAAACUUGAUGGUGAAAAACUCUGUAUAAA |
| | | UUAAUUUAAAAAUUAUUUGGUUUCUCUUUUUAAUUAUUCUGGGGC |
| | | AUAGUCAUUUCUAAAAGUCACUAGUAGAAAGUAUAAUUUCAAGAC |
| | | AGAAUAUUCUAGACAUGCUAGCAGUUUAUAUGUAUUCAUGAGUAA |
| | | UGUGAUAUAUAUUGGGCGCUGGUGAGGAAGGAAGGAGGAAUGAGU |
| | | GACUAUAAGGAUGGUUACCAUAGAAACUUCCUUUUUUACCUAAUUG |
| | | AAGAGAGACUACUACAGAGUGCUAAGCUGCAUGUGUCAUCUUACAC |
| | | UAGAGAGAAAUGGUAAGUUUCUUGUUUUAUUUAAGUUAUGUUUAA |
| | | GCAAGGAAAGGAUUUGUUAUUGAACAGUAUAUUUCAGGAAGGUUA |
| | | GAAAGUGGCGGUUAGGAUAUAUUUUAAAUCUACCUAAAGCAGCAU |
| | | AUUUUAAAAAUUUAAAAGUAUUGGUAUUAAAUUAAGAAAUAGAGG |
| | | ACAGAACUAGACUGAUAGCAGUGACCUAGAACAAUUUGAGAUUAG |
| | | GAAAGUUGUGACCAUGAAUUUAAGGAUUUAUGUGGAUACAAAUUC |
| | | UCCUUUAAAGUGUUUCUUCCCUUAAUAUUUAUCUGACGGUAAUUUU |
| | | UGAGCAGUGAAUUACUUUAUAUAUCUUAAUAGUUUAUUUGGGACC |
| | | AAACACUUAAACAAAAAGUUCUUUAAGUCAUAUAAGCCUUUUCAGG |
| | | AAGCUUGUCUCAUAUUCACUCCCGAGACAUUCACCUGCCAAGUGGC |
| | | CUGAGGAUCAAUCCAGUCCUAGGUUUAUUUUGCAGACUUACAUUCU |
| | | CCCAAGUUAUUCAGCCUCAUAUGACUCCACGGUCGGCUUUACCAAA |
| | | ACAGUUCAGAGUGCACUUUGGCACACAAUUGGGAACAGAACAAUCU |
| | | AAUGUGUGGUUUGGUAUUCCAAGUGGGGUCUUUUUCAGAAUCUCU |
| | | GCACUAGUGUGAGAUGCAAACAUGUUUCCUCAUCUUUCUGGCUUAU |
| | | CCAGUAUGUAGCUAUUUGUGACAUAAUAAAUAUAUACAUAUAUGA |
| | | AAAUA |
| 41 | 6 | GGCGACGACCAGAAGGGGCCCAAGAGAGGGGGCGAGCGACCGAGCG |
| | | CCGCGACGCGGAAGUGAGUGUGGUGUAAAGGAAUUCAUUAGCCAUG |
| | | GAUGUAUUCAUGAAAGGACUUUCAAAGGCCAAGGAGGGAGUUGUG |
| | | GCUGCUGCUGAGAAAACCAAACAGGGUGUGGCAGAAGCAGCAGGAA |
| | | AGACAAAAGAGGGUGUUCUCUAUGUAGGCUCCAAAACCAAGGAGGG |
| | | AGUGGUGCAUGGUGUGGCAACAGUGGCUGAGAAGACCAAAGAGCA |
| | | AGUGACAAAUGUUGGAGGAGCAGUGGUGACGGGUGUGACAGCAGU |
| | | AGCCCAGAAGACAGUGGAGGGAGCAGGGAGCAUUGCAGCAGCCACU |
| | | GGCUUUGUCAAAAAGGACCAGUUGGGCAAGAAUGAAGAAGGAGCCC |
| | | CACAGGAAGGAAUUCUGGAAGAUAUGCCUGUGGAUCCUGACAAUGA |
| | | GGCUUAUGAAAUGCCUUCUGAGGAAGGGUAUCAAGACUACGAACCU |
| | | GAAGCCUAAGAAAUAUCUUUGCUCCCAGUUUCUUGAGAUCUGCUGA |
| | | CAGAUGUUCCAUCCUGUACAAGUGCUCAGUUCCAAUGUGCCCAGUC |
| | | AUGACAUUUCUCAAAGUUUUUACAGUGUAUCUCGAAGUCUUCCAUC |
| | | AGCAGUGAUUGAAGUAUCUGUACCUGCCCCCACUCAGCAUUUCGGU |
| | | GCUUCCCUUUCACUGAAGUGAAUACAUGGUAGCAGGGUCUUUGUGU |
| | | GCUGUGGAUUUUGUGGCUUCAAUCUACGAUGUUAAAACAAAUUAA |
| | | AAACACCUAAGUGACUACCACUUAUUUCUAAAUCCUCACUAUUUUU |
| | | UUGUUGCUGUUGUUCAGAAGUUGUUAGUGAUUUGCUAUCAUAUAU |
| | | UAUAAGAUUUUUAGGUGUCUUUUAAUGAUACUGUCUAAGAAUAAU |
| | | GACGUAUUGUGAAAUUUGUUAAUAUAUAUAAUACUUAAAAAUAUG |
| | | UGAGCAUGAAACUAUGCACCUAUAAAUACUAAAUAUGAAAUUUUA |
| | | CCAUUUUGCGAUGUGUUUUAUUCACUUGUGUUUGUAUAUAAAUGG |
| | | UGAGAAUUAAAAUAAAACGUUAUCUCAUUGCAAAAAUAUUUUAUU |
| | | UUUAUCCCAUCUCACUUUAAUAAUAAAAAUCAUGCUUAUAAGCAAC |
| | | AUGAAUUAAGAACUGACACAAAGGACAAAAAUAUAAAGUUAUUAA |
| | | UAGCCAUUUGAAGAAGGAGGAAUUUUAGAAGAGGUAGAGAAAAUG |
| | | GAACAUUAACCCUACACUCGGAAUUCCCUGAAGCAACACUGCCAGA |
| | | AGUGUGUUUUGGUAUGCACUGGUUCCUUAAGUGGCUGUGAUUAAU |
| | | UAUUGAAAGUGGGGUGUUGAAGACCCCAACUACUAUUGUAGAGUG |
| | | GUCUAUUUCUCCCUUCAAUCCUGUCAAUGUUUGCUUUACGUAUUUU |
| | | GGGGAACUGUUGUUUGAUGUGUAUGUGUUUAUAAUUGUUAUACAU |
| | | UUUUAAUUGAGCCUUUUAUUAACAUAUAUUGUUAUUUUUGUCUCG |
| | | AAAUAAUUUUUUAGUUAAAAUCUAUUUUGUCUGAUAUUGGUGUGA |
| | | AUGCUGUACCUUUCUGACAAUAAAUAAUAUUCGACCAUGAAUAAAA |
| | | AAAAAAAAAAAGUGGGUUCCCGGGAACUAAGCAGUGUAGAAGAUG |
| | | AUUUUGACUACACCCUCCUUAGAGAGCCAUAAGACACAUUAGCACA |
| | | UAUUAGCACAUUCAAGGCUCUGAGAGAAUGUGGUUAACUUUGUUU |
| | | AACUCAGCAUUCCUCACUUUUUUUUUUUAAUCAUCAGAAAUUCUCU |
| | | CUCUCUCUCUCUCUUUUUCUCUCGCUCUCUUUUUUUUUUUUUUUUU |
| | | ACAGGAAAUGCCUUUAAACAUCGUUGGAACUACCAGAGUCACCUUA |
| | | AAGGAGAUCAAUUCUCUAGACUGAUAAAAAUUUCAUGGCCUCCUUU |
| | | AAAUGUUGCCAAAUAUAUGAAUUCUAGGAUUUUUCCUUAGGAAAG |
| | | GUUUUUCUCUUUCAGGGAAGAUCUAUUAACUCCCCAUGGGUGCUGA |
| | | AAAUAAACUUGAUGGUGAAAAACUCUGUAUAAAUUAAUUUAAAAA |
| | | UUAUUUGGUUUCUCUUUUUAAUUAUUCUGGGGCAUAGUCAUUUCU |
| | | AAAAGUCACUAGUAGAAAGUAUAAUUUCAAGACAGAAUAUUCUAG |
| | | ACAUGCUAGCAGUUUAUAUGUAUUCAUGAGUAAUGUGAUAUAUAU |
| | | UGGGCGCUGGUGAGGAAGGAAGGAGGAAUGAGUGACUAUAAGGAU |
| | | GGUUACCAUAGAAACUUCCUUUUUUACCUAAUUGAAGAGAGACUAC |
| | | UACAGAGUGCUAAGCUGCAUGUGUCAUCUUACACUAGAGAGAAAUG |
| | | GUAAGUUUCUUGUUUUAUUUAAGUUAUGUUUAAGCAAGGAAAGGA |
| | | UUUGUUAUUGAACAGUAUAUUUCAGGAAGGUUAGAAAGUGGCGGU |
| | | UAGGAUAUAUUUUAAAUCUACCUAAAGCAGCAUAUUUUAAAAAUU |
| | | UAAAAGUAUUGGUAUUAAAUUAAGAAAUAGAGGACAGAACUAGAC |
| | | UGAUAGCAGUGACCUAGAACAAUUUGAGAUUAGGAAAGUUGUGAC |
| | | CAUGAAUUUAAGGAUUUAUGUGGAUACAAAUUCUCCUUUAAAGUG |
| | | UUUCUUCCCUUAAUAUUUAUCUGACGGUAAUUUUUGAGCAGUGAA |
| | | UUACUUUAUAUAUCUUAAUAGUUUAUUUGGGACCAAACACUUAAA |
| | | CAAAAAGUUCUUUAAGUCAUAUAAGCCUUUUCAGGAAGCUUGUCUC |
| | | AUAUUCACUCCCGAGACAUUCACCUGCCAAGUGGCCUGAGGAUCAA |
| | | UCCAGUCCUAGGUUUAUUUUGCAGACUUACAUUCUCCCAAGUUAUU |
| | | CAGCCUCAUAUGACUCCACGGUCGGCUUUACCAAAACAGUUCAGAG |
| | | UGCACUUUGGCACACAAUUGGGAACAGAACAAUCUAAUGUGUGGUU |
| | | UGGUAUUCCAAGUGGGGUCUUUUUCAGAAUCUCUGCACUAGUGUGA |
| | | GAUGCAAACAUGUUUCCUCAUCUUUCUGGCUUAUCCAGUAUGUAGC |
| | | UAUUUGUGACAUAAUAAAUAUAUACAUAUAUGAAAAUA |
| 42 | 7 | GGCGACGACCAGAAGGGGCCCAAGAGAGGGGGCGAGCGACCGAGCG |
| | | CCGCGACGCGGAAGUGAGGUGCGUGCGGGCUGCAGCGCAGACCCCG |
| | | GCCCGGCCCCUCCGAGAGCGUCCUGGGCGCUCCCUCACGCCUUGCCU |
| | | UCAAGCCUUCUGCCUUUCCACCCUCGUGAGCGGAGAACUGGGAGUG |
| | | GCCAUUCGACGACAGGUUAGCGGGUUUGCCUCCCACUCCCCCAGCC |
| | | UCGCGUCGCCGGCUCACAGCGGCCUCCUCUGGGGACAGUCCCCCCCG |
| | | GGUGCCGCCUCCGCCCUUCCUGUGCGCUCCUUUUCCUUCUUCUUUCC |
| | | UAUUAAAUAUUAUUUGGGAAUUGUUUAAAUUUUUUUUUUAAAAAA |
| | | AGAGAGAGGCGGGGAGGAGUCGGAGUUGUGGAGAAGCAGAGGGAC |
| | | UCAGGGCUGAGAGAUUAGGCUGCUUCUCCGGGAUCCGCUUUUCCCC |
| | | GGGAAACGCGAGGAUGCUCCAUGGAGCUGUGGUGUAAAGGAAUUC |
| | | AUUAGCCAUGGAUGUAUUCAUGAAAGGACUUUCAAAGGCCAAGGA |
| | | GGGAGUUGUGGCUGCUGCUGAGAAAACCAAACAGGGUGUGGCAGA |
| | | AGCAGCAGGAAAGACAAAAGAGGGUGUUCUCUAUGUAGGCUCCAAA |
| | | ACCAAGGAGGGAGUGGUGCAUGGUGUGGCAACAGUGGCUGAGAAG |
| | | ACCAAAGAGCAAGUGACAAAUGUUGGAGGAGCAGUGGUGACGGGU |
| | | GUGACAGCAGUAGCCCAGAAGACAGUGGAGGGAGCAGGGAGCAUUG |
| | | CAGCAGCCACUGGCUUUGUCAAAAAGGACCAGUUGGGCAAGAAUGA |
| | | AGAAGGAGCCCCACAGGAAGGAAUUCUGGAAGAUAUGCCUGUGGAU |
| | | CCUGACAAUGAGGCUUAUGAAAUGCCUUCUGAGGAAGGGUAUCAAG |
| | | ACUACGAACCUGAAGCCUAAGAAAUAUCUUUGCUCCCAGUUUCUUG |
| | | AGAUCUGCUGACAGAUGUUCCAUCCUGUACAAGUGCUCAGUUCCAA |
| | | UGUGCCCAGUCAUGACAUUUCUCAAAGUUUUUACAGUGUAUCUCGA |
| | | AGUCUUCCAUCAGCAGUGAUUGAAGUAUCUGUACCUGCCCCCACUC |
| | | AGCAUUUCGGUGCUUCCCUUUCACUGAAGUGAAUACAUGGUAGCAG |
| | | GGUCUUUGUGUGCUGUGGAUUUUGUGGCUUCAAUCUACGAUGUUA |
| | | AAACAAAUUAAAAACACCUAAGUGACUACCACUUAUUUCUAAAUCC |
| | | UCACUAUUUUUUUGUUGCUGUUGUUCAGAAGUUGUUAGUGAUUUG |
| | | CUAUCAUAUAUUAUAAGAUUUUUAGGUGUCUUUUAAUGAUACUGU |
| | | CUAAGAAUAAUGACGUAUUGUGAAAUUUGUUAAUAUAUAUAAUAC |
| | | UUAAAAAUAUGUGAGCAUGAAACUAUGCACCUAUAAAUACUAAAU |
| | | AUGAAAUUUUACCAUUUUGCGAUGUGUUUUAUUCACUUGUGUUUG |
| | | UAUAUAAAUGGUGAGAAUUAAAAUAAAACGUUAUCUCAUUGCAAA |
| | | AAUAUUUUAUUUUUAUCCCAUCUCACUUUAAUAAUAAAAAUCAUGC |
| | | UUAUAAGCAACAUGAAUUAAGAACUGACACAAAGGACAAAAAUAU |
| | | AAAGUUAUUAAUAGCCAUUUGAAGAAGGAGGAAUUUUAGAAGAGG |
| | | UAGAGAAAAUGGAACAUUAACCCUACACUCGGAAUUCCCUGAAGCA |
| | | ACACUGCCAGAAGUGUGUUUUGGUAUGCACUGGUUCCUUAAGUGGC |
| | | UGUGAUUAAUUAUUGAAAGUGGGGUGUUGAAGACCCCAACUACUA |
| | | UUGUAGAGUGGUCUAUUUCUCCCUUCAAUCCUGUCAAUGUUUGCUU |
| | | UACGUAUUUUGGGGAACUGUUGUUUGAUGUGUAUGUGUUUAUAAU |
| | | UGUUAUACAUUUUUAAUUGAGCCUUUUAUUAACAUAUAUUGUUAU |
| | | UUUUGUCUCGAAAUAAUUUUUUAGUUAAAAUCUAUUUUGUCUGAU |
| | | AUUGGUGUGAAUGCUGUACCUUUCUGACAAUAAAUAAUAUUCGACC |
| | | AUGAAUAAAAAAAAAAAAAAAGUGGGUUCCCGGGAACUAAGCAGU |
| | | GUAGAAGAUGAUUUUGACUACACCCUCCUUAGAGAGCCAUAAGACA |
| | | CAUUAGCACAUAUUAGCACAUUCAAGGCUCUGAGAGAAUGUGGUUA |
| | | ACUUUGUUUAACUCAGCAUUCCUCACUUUUUUUUUUUAAUCAUCAG |
| | | AAAUUCUCUCUCUCUCUCUCUCUUUUUCUCUCGCUCUCUUUUUUUU |
| | | UUUUUUUUUACAGGAAAUGCCUUUAAACAUCGUUGGAACUACCAGA |
| | | GUCACCUUAAAGGAGAUCAAUUCUCUAGACUGAUAAAAAUUUCAUG |
| | | GCCUCCUUUAAAUGUUGCCAAAUAUAUGAAUUCUAGGAUUUUUCCU |
| | | UAGGAAAGGUUUUUCUCUUUCAGGGAAGAUCUAUUAACUCCCCAUG |
| | | GGUGCUGAAAAUAAACUUGAUGGUGAAAAACUCUGUAUAAAUUAA |
| | | UUUAAAAAUUAUUUGGUUUCUCUUUUUAAUUAUUCUGGGGCAUAG |
| | | UCAUUUCUAAAAGUCACUAGUAGAAAGUAUAAUUUCAAGACAGAA |
| | | UAUUCUAGACAUGCUAGCAGUUUAUAUGUAUUCAUGAGUAAUGUG |
| | | AUAUAUAUUGGGCGCUGGUGAGGAAGGAAGGAGGAAUGAGUGACU |
| | | AUAAGGAUGGUUACCAUAGAAACUUCCUUUUUUACCUAAUUGAAG |
| | | AGAGACUACUACAGAGUGCUAAGCUGCAUGUGUCAUCUUACACUAG |
| | | AGAGAAAUGGUAAGUUUCUUGUUUUAUUUAAGUUAUGUUUAAGCA |
| | | AGGAAAGGAUUUGUUAUUGAACAGUAUAUUUCAGGAAGGUUAGAA |
| | | AGUGGCGGUUAGGAUAUAUUUUAAAUCUACCUAAAGCAGCAUAUU |
| | | UUAAAAAUUUAAAAGUAUUGGUAUUAAAUUAAGAAAUAGAGGACA |
| | | GAACUAGACUGAUAGCAGUGACCUAGAACAAUUUGAGAUUAGGAA |
| | | AGUUGUGACCAUGAAUUUAAGGAUUUAUGUGGAUACAAAUUCUCC |
| | | UUUAAAGUGUUUCUUCCCUUAAUAUUUAUCUGACGGUAAUUUUUG |
| | | AGCAGUGAAUUACUUUAUAUAUCUUAAUAGUUUAUUUGGGACCAA |
| | | ACACUUAAACAAAAAGUUCUUUAAGUCAUAUAAGCCUUUUCAGGAA |
| | | GCUUGUCUCAUAUUCACUCCCGAGACAUUCACCUGCCAAGUGGCCU |
| | | GAGGAUCAAUCCAGUCCUAGGUUUAUUUUGCAGACUUACAUUCUCC |
| | | CAAGUUAUUCAGCCUCAUAUGACUCCACGGUCGGCUUUACCAAAAC |
| | | AGUUCAGAGUGCACUUUGGCACACAAUUGGGAACAGAACAAUCUAA |
| | | UGUGUGGUUUGGUAUUCCAAGUGGGGUCUUUUUCAGAAUCUCUGC |
| | | ACUAGUGUGAGAUGCAAACAUGUUUCCUCAUCUUUCUGGCUUAUCC |
| | | AGUAUGUAGCUAUUUGUGACAUAAUAAAUAUAUACAUAUAUGAAA |
| | | AUA |
| 43 | 8 | GAGUGUGAGCGGCGCCUGCUCAGGGUAGAUAGCUGAGGGCGGGGGU |
| | | GGAUGUUGGAUGGAUUAGAACCAUCACACUUGGGCCUGCUGUUUGC |
| | | CUGAGUUUGAACCACACCCCGAUGUGGUGUAAAGGAAUUCAUUAGC |
| | | CAUGGAUGUAUUCAUGAAAGGACUUUCAAAGGCCAAGGAGGGAGU |
| | | UGUGGCUGCUGCUGAGAAAACCAAACAGGGUGUGGCAGAAGCAGCA |
| | | GGAAAGACAAAAGAGGGUGUUCUCUAUGUAGGCUCCAAAACCAAGG |
| | | AGGGAGUGGUGCAUGGUGUGGCAACAGUGGCUGAGAAGACCAAAG |
| | | AGCAAGUGACAAAUGUUGGAGGAGCAGUGGUGACGGGUGUGACAG |
| | | CAGUAGCCCAGAAGACAGUGGAGGGAGCAGGGAGCAUUGCAGCAGC |
| | | CACUGGCUUUGUCAAAAAGGACCAGUUGGGCAAGAAUGAAGAAGG |
| | | AGCCCCACAGGAAGGAAUUCUGGAAGAUAUGCCUGUGGAUCCUGAC |
| | | AAUGAGGCUUAUGAAAUGCCUUCUGAGGAAGGGUAUCAAGACUAC |
| | | GAACCUGAAGCCUAAGAAAUAUCUUUGCUCCCAGUUUCUUGAGAUC |
| | | UGCUGACAGAUGUUCCAUCCUGUACAAGUGCUCAGUUCCAAUGUGC |
| | | CCAGUCAUGACAUUUCUCAAAGUUUUUACAGUGUAUCUCGAAGUCU |
| | | UCCAUCAGCAGUGAUUGAAGUAUCUGUACCUGCCCCCACUCAGCAU |
| | | UUCGGUGCUUCCCUUUCACUGAAGUGAAUACAUGGUAGCAGGGUCU |
| | | UUGUGUGCUGUGGAUUUUGUGGCUUCAAUCUACGAUGUUAAAACA |
| | | AAUUAAAAACACCUAAGUGACUACCACUUAUUUCUAAAUCCUCACU |
| | | AUUUUUUUGUUGCUGUUGUUCAGAAGUUGUUAGUGAUUUGCUAUC |
| | | AUAUAUUAUAAGAUUUUUAGGUGUCUUUUAAUGAUACUGUCUAAG |
| | | AAUAAUGACGUAUUGUGAAAUUUGUUAAUAUAUAUAAUACUUAAA |
| | | AAUAUGUGAGCAUGAAACUAUGCACCUAUAAAUACUAAAUAUGAA |
| | | AUUUUACCAUUUUGCGAUGUGUUUUAUUCACUUGUGUUUGUAUAU |
| | | AAAUGGUGAGAAUUAAAAUAAAACGUUAUCUCAUUGCAAAAAUAU |
| | | UUUAUUUUUAUCCCAUCUCACUUUAAUAAUAAAAAUCAUGCUUAUA |
| | | AGCAACAUGAAUUAAGAACUGACACAAAGGACAAAAAUAUAAAGU |
| | | UAUUAAUAGCCAUUUGAAGAAGGAGGAAUUUUAGAAGAGGUAGAG |
| | | AAAAUGGAACAUUAACCCUACACUCGGAAUUCCCUGAAGCAACACU |
| | | GCCAGAAGUGUGUUUUGGUAUGCACUGGUUCCUUAAGUGGCUGUG |
| | | AUUAAUUAUUGAAAGUGGGGUGUUGAAGACCCCAACUACUAUUGU |
| | | AGAGUGGUCUAUUUCUCCCUUCAAUCCUGUCAAUGUUUGCUUUACG |
| | | UAUUUUGGGGAACUGUUGUUUGAUGUGUAUGUGUUUAUAAUUGUU |
| | | AUACAUUUUUAAUUGAGCCUUUUAUUAACAUAUAUUGUUAUUUUU |
| | | GUCUCGAAAUAAUUUUUUAGUUAAAAUCUAUUUUGUCUGAUAUUG |
| | | GUGUGAAUGCUGUACCUUUCUGACAAUAAAUAAUAUUCGACCAUGA |
| | | AUAAAAAAAAAAAAAAAGUGGGUUCCCGGGAACUAAGCAGUGUAG |
| | | AAGAUGAUUUUGACUACACCCUCCUUAGAGAGCCAUAAGACACAUU |
| | | AGCACAUAUUAGCACAUUCAAGGCUCUGAGAGAAUGUGGUUAACUU |
| | | UGUUUAACUCAGCAUUCCUCACUUUUUUUUUUUAAUCAUCAGAAAU |
| | | UCUCUCUCUCUCUCUCUCUUUUUCUCUCGCUCUCUUUUUUUUUUUU |
| | | UUUUUACAGGAAAUGCCUUUAAACAUCGUUGGAACUACCAGAGUCA |
| | | CCUUAAAGGAGAUCAAUUCUCUAGACUGAUAAAAAUUUCAUGGCCU |
| | | CCUUUAAAUGUUGCCAAAUAUAUGAAUUCUAGGAUUUUUCCUUAG |
| | | GAAAGGUUUUUCUCUUUCAGGGAAGAUCUAUUAACUCCCCAUGGGU |
| | | GCUGAAAAUAAACUUGAUGGUGAAAAACUCUGUAUAAAUUAAUUU |
| | | AAAAAUUAUUUGGUUUCUCUUUUUAAUUAUUCUGGGGCAUAGUCA |
| | | UUUCUAAAAGUCACUAGUAGAAAGUAUAAUUUCAAGACAGAAUAU |
| | | UCUAGACAUGCUAGCAGUUUAUAUGUAUUCAUGAGUAAUGUGAUA |
| | | UAUAUUGGGCGCUGGUGAGGAAGGAAGGAGGAAUGAGUGACUAUA |
| | | AGGAUGGUUACCAUAGAAACUUCCUUUUUUACCUAAUUGAAGAGA |
| | | GACUACUACAGAGUGCUAAGCUGCAUGUGUCAUCUUACACUAGAGA |
| | | GAAAUGGUAAGUUUCUUGUUUUAUUUAAGUUAUGUUUAAGCAAGG |
| | | AAAGGAUUUGUUAUUGAACAGUAUAUUUCAGGAAGGUUAGAAAGU |
| | | GGCGGUUAGGAUAUAUUUUAAAUCUACCUAAAGCAGCAUAUUUUA |
| | | AAAAUUUAAAAGUAUUGGUAUUAAAUUAAGAAAUAGAGGACAGAA |
| | | CUAGACUGAUAGCAGUGACCUAGAACAAUUUGAGAUUAGGAAAGU |
| | | UGUGACCAUGAAUUUAAGGAUUUAUGUGGAUACAAAUUCUCCUUU |
| | | AAAGUGUUUCUUCCCUUAAUAUUUAUCUGACGGUAAUUUUUGAGC |
| | | AGUGAAUUACUUUAUAUAUCUUAAUAGUUUAUUUGGGACCAAACA |
| | | CUUAAACAAAAAGUUCUUUAAGUCAUAUAAGCCUUUUCAGGAAGCU |
| | | UGUCUCAUAUUCACUCCCGAGACAUUCACCUGCCAAGUGGCCUGAG |
| | | GAUCAAUCCAGUCCUAGGUUUAUUUUGCAGACUUACAUUCUCCCAA |
| | | GUUAUUCAGCCUCAUAUGACUCCACGGUCGGCUUUACCAAAACAGU |
| | | UCAGAGUGCACUUUGGCACACAAUUGGGAACAGAACAAUCUAAUGU |
| | | GUGGUUUGGUAUUCCAAGUGGGGUCUUUUUCAGAAUCUCUGCACUA |
| | | GUGUGAGAUGCAAACAUGUUUCCUCAUCUUUCUGGCUUAUCCAGUA |
| | | UGUAGCUAUUUGUGACAUAAUAAAUAUAUACAUAUAUGAAAAUA |
| 44 | 9 | GCUCUAAUUCUCUGCACCUUCUCAAGCAUUGUGCAGAUUGGUUUUC |
| | | UGGAUUAUCAGCCUGAAGGACAAAACGAAGAAACAGCCAUUAGCUC |
| | | CUGUCUCCCAUUGUCUGAGAGCUGCCACUAGGAUAUUAACUUCCUG |
| | | AAAUUCUGCAGAAAUCUCCUCUUACUUUGGCACUGGAGAUGCCCAU |
| | | ACGCAGAAAGCAAAAAGGCACAGCAUAUUUAAGGAAGCUCAUAAGA |
| | | AACAGUGCAUCCAGAAGUGGCGAGAAUUGGAGGAAUGGACAUGAG |
| | | ACUCUAAGAACCAGCGCCUUUGAUGUUCCUUUUGAUCUGUUAUGUA |
| | | GCUCUUCUUGUACACAGAAUGAAGAAGGAGCCCCACAGGAAGGAAU |
| | | UCUGGAAGAUAUGCCUGUGGAUCCUGACAAUGAGGCUUAUGAAAU |
| | | GCCUUCUGAGGAAGGGUAUCAAGACUACGAACCUGAAGCCUAAGAA |
| | | AUAUCUUUGCUCCCAGUUUCUUGAGAUCUGCUGACAGAUGUUCCAU |
| | | CCUGUACAAGUGCUCAGUUCCAAUGUGCCCAGUCAUGACAUUUCUC |
| | | AAAGUUUUUACAGUGUAUCUCGAAGUCUUCCAUCAGCAGUGAUUGA |
| | | AGUAUCUGUACCUGCCCCCACUCAGCAUUUCGGUGCUUCCCUUUCA |
| | | CUGAAGUGAAUACAUGGUAGCAGGGUCUUUGUGUGCUGUGGAUUU |
| | | UGUGGCUUCAAUCUACGAUGUUAAAACAAAUUAAAAACACCUAAGU |
| | | GACUACCACUUAUUUCUAAAUCCUCACUAUUUUUUUGUUGCUGUUG |
| | | UUCAGAAGUUGUUAGUGAUUUGCUAUCAUAUAUUAUAAGAUUUUU |
| | | AGGUGUCUUUUAAUGAUACUGUCUAAGAAUAAUGACGUAUUGUGA |
| | | AAUUUGUUAAUAUAUAUAAUACUUAAAAAUAUGUGAGCAUGAAAC |
| | | UAUGCACCUAUAAAUACUAAAUAUGAAAUUUUACCAUUUUGCGAU |
| | | GUGUUUUAUUCACUUGUGUUUGUAUAUAAAUGGUGAGAAUUAAAA |
| | | UAAAACGUUAUCUCAUUGCAAAAAUAUUUUAUUUUUAUCCCAUCUC |
| | | ACUUUAAUAAUAAAAAUCAUGCUUAUAAGCAACAUGAAUUAAGAA |
| | | CUGACACAAAGGACAAAAAUAUAAAGUUAUUAAUAGCCAUUUGAA |
| | | GAAGGAGGAAUUUUAGAAGAGGUAGAGAAAAUGGAACAUUAACCC |
| | | UACACUCGGAAUUCCCUGAAGCAACACUGCCAGAAGUGUGUUUUGG |
| | | UAUGCACUGGUUCCUUAAGUGGCUGUGAUUAAUUAUUGAAAGUGG |
| | | GGUGUUGAAGACCCCAACUACUAUUGUAGAGUGGUCUAUUUCUCCC |
| | | UUCAAUCCUGUCAAUGUUUGCUUUACGUAUUUUGGGGAACUGUUG |
| | | UUUGAUGUGUAUGUGUUUAUAAUUGUUAUACAUUUUUAAUUGAGC |
| | | CUUUUAUUAACAUAUAUUGUUAUUUUUGUCUCGAAAUAAUUUUUU |
| | | AGUUAAAAUCUAUUUUGUCUGAUAUUGGUGUGAAUGCUGUACCUU |
| | | UCUGACAAUAAAUAAUAUUCGACCAUGAAUAAAAAAAAAAAAAAA |
| | | GUGGGUUCCCGGGAACUAAGCAGUGUAGAAGAUGAUUUUGACUAC |
| | | ACCCUCCUUAGAGAGCCAUAAGACACAUUAGCACAUAUUAGCACAU |
| | | UCAAGGCUCUGAGAGAAUGUGGUUAACUUUGUUUAACUCAGCAUUC |
| | | CUCACUUUUUUUUUUUAAUCAUCAGAAAUUCUCUCUCUCUCUCUCU |
| | | CUUUUUCUCUCGCUCUCUUUUUUUUUUUUUUUUUACAGGAAAUGCC |
| | | UUUAAACAUCGUUGGAACUACCAGAGUCACCUUAAAGGAGAUCAAU |
| | | UCUCUAGACUGAUAAAAAUUUCAUGGCCUCCUUUAAAUGUUGCCAA |
| | | AUAUAUGAAUUCUAGGAUUUUUCCUUAGGAAAGGUUUUUCUCUUU |
| | | CAGGGAAGAUCUAUUAACUCCCCAUGGGUGCUGAAAAUAAACUUGA |
| | | UGGUGAAAAACUCUGUAUAAAUUAAUUUAAAAAUUAUUUGGUUUC |
| | | UCUUUUUAAUUAUUCUGGGGCAUAGUCAUUUCUAAAAGUCACUAG |
| | | UAGAAAGUAUAAUUUCAAGACAGAAUAUUCUAGACAUGCUAGCAG |
| | | UUUAUAUGUAUUCAUGAGUAAUGUGAUAUAUAUUGGGCGCUGGUG |
| | | AGGAAGGAAGGAGGAAUGAGUGACUAUAAGGAUGGUUACCAUAGA |
| | | AACUUCCUUUUUUACCUAAUUGAAGAGAGACUACUACAGAGUGCUA |
| | | AGCUGCAUGUGUCAUCUUACACUAGAGAGAAAUGGUAAGUUUCUU |
| | | GUUUUAUUUAAGUUAUGUUUAAGCAAGGAAAGGAUUUGUUAUUGA |
| | | ACAGUAUAUUUCAGGAAGGUUAGAAAGUGGCGGUUAGGAUAUAUU |
| | | UUAAAUCUACCUAAAGCAGCAUAUUUUAAAAAUUUAAAAGUAUUG |
| | | GUAUUAAAUUAAGAAAUAGAGGACAGAACUAGACUGAUAGCAGUG |
| | | ACCUAGAACAAUUUGAGAUUAGGAAAGUUGUGACCAUGAAUUUAA |
| | | GGAUUUAUGUGGAUACAAAUUCUCCUUUAAAGUGUUUCUUCCCUUA |
| | | AUAUUUAUCUGACGGUAAUUUUUGAGCAGUGAAUUACUUUAUAUA |
| | | UCUUAAUAGUUUAUUUGGGACCAAACACUUAAACAAAAAGUUCUU |
| | | UAAGUCAUAUAAGCCUUUUCAGGAAGCUUGUCUCAUAUUCACUCCC |
| | | GAGACAUUCACCUGCCAAGUGGCCUGAGGAUCAAUCCAGUCCUAGG |
| | | UUUAUUUUGCAGACUUACAUUCUCCCAAGUUAUUCAGCCUCAUAUG |
| | | ACUCCACGGUCGGCUUUACCAAAACAGUUCAGAGUGCACUUUGGCA |
| | | CACAAUUGGGAACAGAACAAUCUAAUGUGUGGUUUGGUAUUCCAA |
| | | GUGGGGUCUUUUUCAGAAUCUCUGCACUAGUGUGAGAUGCAAACAU |
| | | GUUUCCUCAUCUUUCUGGCUUAUCCAGUAUGUAGCUAUUUGUGACA |
| | | UAAUAAAUAUAUACAUAUAUGAAAAUA |

In some cases, a region of Alpha-synuclein polypeptide can be targeted utilizing compositions provided herein. Suitable regions include but are not limited to a N-terminal A2 lipid-binding alpha-helix domain, a Non-amyloid β component (NAC) domain, or a C-terminal acidic domain.

In some aspects, an alpha-synuclein polypeptide sequence is targeted, complete polypeptide sequences are shown in TABLE 7. In some cases, any one of the residues of an isoform sequence may be targeted utilizing the compositions and methods provided herein. In some cases, a target residue may be located among residues 1-10, 10-20, 20-40, 40-60, 60-80, 80-100, 100-120, or 120-140, overlapping portions thereof, and combinations thereof.

TABLE 7
Human alpha-synuclein polypeptide
sequences associated with isoform of TABLE 6

| SEQ | | |
|---|---|---|
| ID | | |
| NO | Isoform | SNCA Polypeptide Sequence |
| 45 | Isoform 1-3; | MDVFMKGLSKAKEGVVAAAEKTKQGVAEAAG |
| | 5-8 | KTKEGVLYVGSKTKEGVVHGVATVAEKTKEQ |
| | | VTNVGGAVVTGVTAVAQKTVEGAGSIAAATG |
| | | FVKKDQLGKNEEGAPQEGILEDMPVDPDNEA |
| | | YEMPSEEGYQDYEPEA |
| 46 | Isoform 4 | MDVFMKGLSKAKEGVVAAAEKTKQGVAEAAG |
| | | KTKEGVLYVGSKTKEGVVHGVATVAEKTKEQ |
| | | VTNVGGAVVTGVTAVAQKTVEGAGSIAAATG |
| | | FVKKDQLGKEGYQDYEPEA |
| 47 | Isoform 9 | MPIRRKQKGTAYLRKLIRNSASRSGENWRNG |
| | | HETLRTSAFDVPFDLLCSSSCTQNEEGAPQE |
| | | GILEDMPVDPDNEAYEMPSEEGYQDYEPEA |

Exemplary regions that can be targeted utilizing compositions provided herein can include but are not limited to a target residue of an SNCA polypeptide sequence is any one of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and/or 140. In some cases, exemplary residues that can be targeted can comprise M1, V40, K85, S125, K129, or any combination thereof.

In some embodiments, the editing of a base of the SNCA mRNA results in a decreased gene translation of the SNCA polypeptide. In other cases, the editing of a base of the 5′UTR of the SNCA mRNA results in a decreased gene translation of the SNCA polypeptide. The decreased gene translation of the SNCA polypeptide can be measured by an in vitro assay. Such in vitro assay can comprise an in vitro translation assay. An in vitro translation assay can comprise a cell extract. A cell extract can comprise rabbit reticulocyte lysate, wheat germ extract, insect cells, yeast Kluyveromyces, or E coli cell-free extract. An in vitro translation assay can comprise mixing a cell extract with a nucleic acid template, ATP, and amino acids. A nucleic acid template can comprise a mRNA template or a cDNA template. A nucleic acid template can comprise a mRNA sequence listed in TABLE 6. A nucleic acid template can comprise a cDNA sequence complementary to the mRNA sequence listed in TABLE 6. When using an in vitro translation system with a cDNA template, the cDNA can be converted to a mRNA by in vitro transcription. A cDNA can be maintained in a circular vector. A cDNA can be maintained as a linear sequence.

In some cases, the engineered polynucleotide capable of facilitating a modification of a base of a nucleotide comprised in a SNCA pre-mRNA or mRNA can down-regulate or knockdown the expression of the SNCA protein. In some embodiments, the editing of the SNCA pre-mRNA or mRNA can inhibit or down-regulate the splicing, mRNA export, mRNA localization, mRNA stability, mRNA degradation, polyadenylation, 5′cap modification, assembly of messenger ribonucleoprotein (mRNP), or translation of the SNCA pre-mRNA or mRNA. In some cases, the editing of the SNCA pre-mRNA or mRNA can result in a generation of an edited SNCA mRNA encoding a polypeptide comprising at least one amino acid substitution compared to a polypeptide encoded by an unedited polynucleotide. In other cases, the editing of the SNCA pre-mRNA or mRNA may not result in a generation of an edited SNCA mRNA encoding a polypeptide comprising at least one amino acid substitution compared to a polypeptide encoded by an unedited polynucleotide. In some cases, a modified SNCA mRNA can encode for a SNCA polypeptide, with or without an amino acid substitution, having lower amount than a SNCA protein encoded by an unedited SNCA mRNA. In some cases, the amount of SNCA polypeptide, with or without an amino acid substitution, can be measured by a SNCA ELISA. In an embodiment, the lower abundance of SNCA polypeptide or fragment thereof can be least or at most about: 1-fold, 8-fold, 15-fold, 22-fold, 29-fold, 36-fold, 43-fold, 50-fold, 57-fold, 64-fold, 71-fold, 78-fold, 85-fold, 92-fold, 99-fold, 106-fold, 113-fold, 120-fold, 127-fold, 134-fold, 141-fold, 148-fold, 155-fold, 162-fold, 169-fold, 176-fold, 183-fold, 190-fold, 197-fold, 204-fold, 211-fold, 218-fold, 225-fold, 232-fold, 239-fold, 246-fold, 253-fold, 260-fold, 267-fold, 274-fold, 281-fold, 288-fold, 295-fold, or up to about 350-fold.

In an embodiment, the lower abundance of SNCA polypeptide or fragment thereof can be least or at most about: 0.1-0.2 fold, 0.19-0.3 fold, 0.29-0.4 fold, 0.39-0.5 fold, 0.49-0.6 fold, 0.59-0.7 fold, 0.69-0.8 fold, 0.79-09 fold, 0.89-1 fold, 0.99-1 fold, 0.1-0.3 fold, 0.1-0.4 fold, 0.1-0.5 fold, 0.1-0.6 fold, 0.1-0.7 fold, 0.1-0.8 fold, 0.1-0.9 fold, 1-20-fold, 2-21-fold, 3-22-fold, 4-23-fold, 5-24-fold, 6-25-fold, 7-26-fold, 8-27-fold, 9-28-fold, 10-29-fold, 11-30-fold, 12-31-fold, 13-32-fold, 14-33-fold, 15-34-fold, 16-35-fold, 17-36-fold, 18-37-fold, 19-38-fold, 20-39-fold, 21-40-fold, 22-41-fold, 23-42-fold, 24-43-fold, 25-44-fold, 26-45-fold, 27-46-fold, 28-47-fold, 29-48-fold, 30-49-fold, 31-50-fold, 32-51-fold, 33-52-fold, 34-53-fold, 35-54-fold, 36-55-fold, 37-56-fold, 38-57-fold, 39-58-fold, 40-59-fold, 41-60-fold, 42-61-fold, 43-62-fold, 44-63-fold, 45-64-fold, 46-65-fold, 47-66-fold, 48-67-fold, 49-68-fold, 50-69-fold, 51-70-fold, 52-71-fold, 53-72-fold, 54-73-fold, 55-74-fold, 56-75-fold, 57-76-fold, 58-77-fold, 59-78-fold, 60-79-fold, 61-80-fold, 62-81-fold, 63-82-fold, 64-83-fold, 65-84-fold, 66-85-fold, 67-86-fold, 68-87-fold, 69-88-fold, 70-89-fold, 71-90-fold, 72-91-fold, 73-92-fold, 74-93-fold, 75-94-fold, 76-95-fold, 77-96-fold, 78-97-fold, 79-98-fold, 80-99-fold, or 81-100-fold decrease in the protein level of SNCA, as compared to that encoded by the unedited SNCA mRNA.

### Genome Editing of APP

In some embodiments, the APP gene can be altered using genome editing. Genome editing can comprise a CRISPR/Cas associated protein, RNA guided endonuclease, zinc finger nuclease, transcription activator-like effector nuclease (TALEN), meganuclease, functional portion of any of these, fusion protein of any of these, or any combination thereof. In some embodiments, a CRISPR/Cas associated protein can comprise a CRISPR/Cas endonuclease. In some embodiments, a CRISPR/Cas associated protein can comprise class 1 or class 2 CRISPR/Cas protein. A class 2 CRISPR/Cas associated protein can comprise a type II CRISPR/Cas protein, a type V CRISPR/Cas protein, a type VI CRISPR/Cas protein. A CRISPR/Cas associated protein can comprise a Cas9 protein, Cas 12 protein, functional portion of any of these, fusion protein of any of these, or any combinations thereof. A CRISPR/Cas associated protein can comprise a wildtype or a variant CRISPR/Cas associated protein, functional portion of any of these, fusion protein of any of these, or any combinations thereof. A CRISPR/Cas associated protein can comprise a base editor. A base editor can comprise a cytidine deaminase, a deoxyadenosine deaminase, functional portion of any of these, fusion protein of any of these, or any combinations thereof. A CRISPR/Cas associated protein can comprise a reverse transcriptase. A reverse transcriptase can comprise a Moloney murine leukemia virus (M-MLV) reverse transcriptase or an Avian Myeloblastosis Virus (AMV) reverse transcriptase.

A CRISPR/Cas associated protein as described herein are targeted to a specific target DNA sequence in a genome by a guide RNA to which it is bound. The guide RNA comprises a sequence that is complementary to a target sequence within the target DNA, thus targeting the bound CRISPR/Cas protein to a specific location within the target DNA (the target sequence). A CRISPR/Cas associated protein, when targeted to the specific target DNA sequence, can create a single-strand break, two single-strand breaks, a double-strand break, two double-strand breaks, or any combinations thereof in the genome. A CRISPR/Cas associated protein, when targeted to the specific target DNA sequence, may not create any breaks in the genome. A CRISPR/Cas associated protein-guide RNA complex can make a blunt-ended double-stranded break, a 1-base pair (bp) staggered cut, a 2-bp staggered cut, a staggered cut with more than 2 base pairs, or any combination thereof in the genome. A double-strand DNA break can be repaired by end-joining mechanism or homologous directed repair. A double-strand DNA break can also be repaired by end-joining mechanism or homologous directed repair with a double strand donor DNA or a single-stranded oligonucleotide donor DNA. An edit in the genome can comprise stochastic or pre-selected insertions, deletions, base substitutions, inversion, chromosomal translocation, insertion.

A guide RNA can comprise a single guide RNA (sgRNA), a double guide RNA, or an engineered prime editing guide RNA (pegRNA). A guide RNA can comprise a crRNA and a tracrRNA. A crRNA can comprise a targeting sequence that hybridizes to a target sequence in the target DNA or locus. A tracrRNA can comprise a sequence that can form a stem-loop structure. Such a stem-loop structure can bind a CRISPR/Cas associated protein to activate the nuclease activity of the CRISPR/Cas associated protein. A sgRNA can comprise a crRNA and a tracrRNA in one RNA molecule. A double guide RNA can comprise a crRNA and a tracrRNA in two RNA molecules. A pegRNA can comprise a sequence that comprises a pre-selected edit or sequence in the genome. In such editing, the pre-selected sequence hybridizes to a cut and liberated 3′ end of a nicked/cut DNA strand to form a primer-template complex, wherein the cut, liberated, and hybridized 3′ end of the nicked/cut DNA strand can serve as a primer while the pre-selected edit or sequence of the pegRNA can serve as a template for the subsequent reactions, including but not limited to reverse transcription.

Suppressing of Pathogenic Mutations with a Suppressor tRNA

In some embodiments, the translation of a mRNA with a pathogenic mutation can be read through by an engineered tRNA with an engineered anticodon region. For example, a wild type codon can code for amino acid A while a pathogenic mutation can create a mutated codon that codes for a premature stop codon. An engineered tRNA can have an engineered anticodon region capable of recognizing and reading through the stop codon. In some embodiments, the anti-codon loop, arm length, tRNA-ribosome affinity in position 32/38, or other tRNA features can be optimized to increase the codon usage of the engineered tRNA. In other cases, the promoter, copy number, or other features can also be optimized to increase the codon usage of the engineered tRNA. In some embodiments, engineered polynucleotides of the present disclosure can be administered to a subject in need thereof in combination with a suppressor tRNA.

### Manipulating the Phosphorylation of the APP, Tau, SNCA Polypeptide by RNA Editing

In some embodiments, RNA editing of a target RNA can increase or decrease the amount of phosphorylation of a polypeptide encoded by the edited target RNA, compared to a polypeptide encoded by an unedited target RNA. RNA editing can effect an amino substitution that results in a deletion of an endogenous phosphorylation site. An endogenous phosphorylation can be identified by a polypeptide-specific antibody, phosphorylated site-specific antibody, phosphorylated fragment-specific antibody, a phosphorylated polypeptide-specific antibody, any derivatives herein and thereof, or any combinations herein and thereof. In other cases, a phosphorylation site of a polypeptide can be identified by a computer algorithm. A phosphorylation identification computer algorithm can comprise DISPHOS 1.3, ELM, GPS, NetPhorest, NetPhos, NetworKIN, P3DB, PHOSIDA, PhosphoregDB, PhosphoSitePlus, PREDIKIN, RLIMS, Scansite, any derivatives herein and thereof, or any combinations herein and thereof. In other cases, a phosphorylation site of a polypeptide can comprise mass-spectroscopy any derivatives herein and thereof, or any combinations herein and thereof. In some embodiments, RNA editing can effect an amino substitution outside of an endogenous phosphorylation site. An amino substitution outside of an endogenous phosphorylation site can increase or decrease a phosphorylation amount of the endogenous phosphorylation site. In other cases, an amino substitution may create a phosphorylation site. A created phosphorylation site can be identified by means that can identify an endogenous phosphorylation described herein and thereof. In some embodiments, an amino acid substitution can mimic a phosphorylation. A mimic of a phosphorylation can comprise a phosphomimetic. A phosphomimetic can comprise an amino substitution to aspartic acid. Aspartic acid can mimic a serine with phosphorylation.

A phosphorylation of a polypeptide can increase or decrease the enzymatic activity, stability, intracellular localization, extracellular localization, protein-binding activity, oligomerization activity, enzymatic processing, secretion, antimicrobial activity, degradation, chemical binding, or any endogenous or artificial activity of the polypeptide. In some embodiments, a phosphorylation of a polypeptide can increase or decrease the enzymatic activity, stability, intracellular localization, extracellular localization, protein-binding activity, oligomerization activity, enzymatic processing, secretion, antimicrobial activity, degradation, chemical binding, or any endogenous or artificial activity of a different polypeptide.

A phosphorylation of a polypeptide can be measured by a western blot with a polypeptide-specific antibody, phosphorylated site-specific antibody, phosphorylated fragment-specific antibody, a phosphorylated polypeptide-specific antibody. In other cases, a phosphorylation of a polypeptide can be measure by mass spectroscopy. A phosphorylation of a polypeptide can also be measured by an endogenous function of the polypeptide described herein and thereof. In some cases, a phosphorylation of a polypeptide can be measured by an in vitro assay with A protein is first phosphorylated using radioactive 32P-labeled ATP.

Phosphorylation of an APP or Abeta peptide can increaser or decrease an enzymatic cleavage of the APP or Abeta peptide by alpha, beta, beta′, gamma-secretase, any derivatives thereof, or any combinations thereof. Phosphorylation of an APP or Abeta peptide can also increaser or decrease an enzymatic cleavage of the APP or Abeta peptide by BACE1, cathepsin B or Meprin beta. In other cases, phosphorylation of an APP or Abeta peptide can increaser or decrease an oligomerization or the APP/Abeta binding ability of the APP or Abeta peptide. In some cases, phosphorylation of an APP or Abeta peptide can increaser or decrease the formation, aggregation, or stability of a beta amyloid plaque.

A phosphorylation site can comprise position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, or any combinations thereof of SEQ ID NO: 2, 14, or 15. In some cases, A phosphorylation site can be from position 1 to 10, from position 9 to 20, from position 19 to 30, from position 29 to 40, from position 39 to 50, from position 49 to 60, from position 59 to 70, from position 69 to 80, from position 79 to 90, from position 89 to 100, from position 99 to 110, from position 109 to 120, from position 119 to 130, from position 129 to 140, from position 139 to 150, from position 149 to 160, from position 159 to 170, from position 169 to 180, from position 179 to 190, from position 189 to 200, from position 199 to 210, from position 209 to 220, from position 219 to 230, from position 229 to 240, from position 239 to 250, from position 249 to 260, from position 259 to 270, from position 269 to 280, from position 279 to 290, from position 289 to 300, from position 299 to 310, from position 309 to 320, from position 319 to 330, from position 329 to 340, from position 339 to 350, from position 349 to 360, from position 359 to 370, from position 369 to 380, from position 379 to 390, from position 389 to 400, from position 399 to 410, from position 409 to 420, from position 419 to 430, from position 429 to 440, from position 439 to 450, from position 449 to 460, from position 459 to 470, from position 469 to 480, from position 479 to 490, from position 489 to 500, from position 499 to 510, from position 509 to 520, from position 519 to 530, from position 529 to 540, from position 539 to 550, from position 549 to 560, from position 559 to 570, from position 569 to 580, from position 579 to 590, from position 589 to 600, from position 599 to 610, from position 609 to 620, from position 619 to 630, from position 629 to 640, from position 639 to 650, from position 649 to 660, from position 659 to 670, from position 669 to 680, from position 679 to 690, from position 689 to 700, from position 699 to 710, from position 709 to 720, from position 719 to 730, from position 729 to 740, from position 739 to 750, from position 749 to 760, from position 759 to 770, or any combinations herein and thereof SEQ ID NO: 2, 14, or 15. A phosphorylation site can comprise position threonine 668 (T668), serine 198 (S198), S8, S26, S206, T10, T111, Y115, T153, S184, S185, S198, T205, S206, S208, T217, S262, S285 T381, S441, S679, S697, Y728, T729, S730, T743, Y757, T761, Y762, or any combinations herein and thereof of SEQ ID NO: 2.

Phosphorylation of a Tau polypeptide can increase or decrease formation, aggregation, or stability of neurofibrillary tangles (NFTs). In some cases, a phosphorylation site can comprise position of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, or any combinations herein and thereof of SEQ ID NO: 28-35. In some cases, a phosphorylation site can comprise from position 1 to 50, from position 49 to 100, from position 99 to 150, from position 149 to 200, from position 199 to 250, from position 249 to 300, from position 299 to 350, from position 349 to 400, from position 399 to 450, from position 449 to 500, from position 499 to 550, from position 549 to 600, from position 599 to 650, from position 649 to 700, from position 699 to 750, from position 749 to 800, from position 799 to 850, from position 849 to 862, or any combinations herein and thereof of SEQ ID NO: 28-35. A phosphorylation can comprise Y18, T39, S46, T50, T52, S56, S61, S64, T69, T111, S131, T149, T153, S171, T173, T175, T181, S191, S195, S199, S202, S205, S208, S210, S212, S214, S217, S231, S232, S235, S237, S238, S241, S258, S285, S289, S316, S320, S352, S355, S369, T373, T386, S388, S411, T466, T470, S396, S400, S404, S409, S412, T498, S501, S502, S508, S512, Y514, S515, S516, T522, S525, S527, T529, S531, T534, T548, S552, S554, S555, S558, S579, T580, S602, S610, S622, Y627, S633, T636, S641, S673, T694, Y711, S713, S717, T720, S721, S726, S729, S730, T731, S733, S739, T744, or any combinations herein and thereof of SEQ ID NO: 28-35.

Phosphorylation of a SNCA polypeptide can increase or decrease formation, aggregation, or stability of the SNCA polypeptide. In some cases, a phosphorylation site can comprise position of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or any combinations herein and thereof of SEQ ID NO: 45-47. A phosphorylation site can be from position 1 to 10, from position 9 to 20, from position 19 to 30, from position 29 to 40, from position 39 to 50, from position 49 to 60, from position 59 to 70, from position 69 to 80, from position 79 to 90, from position 89 to 100, from position 99 to 110, from position 109 to 120, from position 119 to 130, from position 129 to 140 or any combinations herein and thereof of SEQ ID NO: 45-47. A phosphorylation can comprise Y39, S42, S87, Y125, S129, Y133, Y136, or any combinations herein and thereof of SEQ ID NO: 45-47.

### Vectors

The present disclosure also provides for vectors that encode for the engineered guide RNAs disclosed herein.

The compositions provided herein can be delivered by any suitable means. In some cases, a suitable means comprises a vector. Any vector system can be used utilized, including but not limited to: plasmid vectors, minicircle vectors, linear DNA vectors, doggy bone vectors, retroviral vectors, lentiviral vectors, adenovirus vectors, poxvirus vectors; herpesvirus vectors and adeno-associated virus vectors, a liposome, a nanoparticle, an exosome, an extracellular vesicle, a nanomesh, modified versions thereof, good manufacturing practices versions thereof, chimeras thereof, and any combination thereof. In some cases, a vector can be used to introduce a polynucleotide provided herein. In some cases, the polynucleotide comprises a targeting sequence that hybridizes to a region of an RNA provided herein. In some embodiments, a nanoparticle vector can comprise a polymeric-based nanoparticle, an amino lipid-based nanoparticle, a metallic nanoparticle (such as gold-based nanoparticle), a portion of any of these, or any combination thereof.

Vectors provided herein can be used to deliver polynucleotide compositions provided herein. In some cases, at least about 2, 3, 4, or up to 5 different polynucleotides are delivered using a single vector. In some cases, multiple vectors are delivered. In some cases, multiple vector delivery can be co-current or sequential. In some cases, at least two engineered polynucleotides are delivered in a single vector. In other cases, at least two engineered polynucleotides are delivered on separate vectors. Engineered polynucleotides may also be delivered as naked polynucleotides. Any combination of vector and/or a non-vector approach can be taken.

A vector can be employed to deliver a nucleic acid. A vector can comprise DNA, such as double stranded DNA or single stranded DNA. A vector can comprise RNA. In some cases, the RNA can comprise a base modification. The vector can comprise a recombinant vector. The vector can be a vector that is modified from a naturally occurring vector. The vector can comprise at least a portion of a non-naturally occurring vector. Any vector can be utilized. A viral vector can comprise an adenoviral vector, an adeno-associated viral vector (AAV), a lentiviral vector, a retroviral vector, a portion of any of these, or any combination thereof. In some cases, a vector can comprise an AAV vector. A vector can be modified to include a modified VP1 protein (such as an AAV vector modified to include a VP1 protein). In an aspect an AAV vector is a recombinant AAV (rAAV) vector. rAAVs can be composed of substantially similar capsid sequence and structure as found in wild-type AAVs (wtAAVs). However, rAAVs encapsidate genomes that are substantially devoid of AAV protein-coding sequences and have therapeutic gene expression cassettes, such as subject polynucleotides, designed in their place. In some cases, sequences of viral origin can be the ITRs, which may be needed to guide genome replication and packaging during vector production. Suitable AAV vectors can be selected from any AAV serotype or combination of serotypes. For example, an AAV vector can be any one of: AAV1, AAV2, AAV3, AAV4, AAV5, AAV6, AAV7, AAV8, AAV9, AAV 10, AAV11, AAV12, or any combination thereof. In some cases, a vector is selected based on its natural tropism. In some cases, a vector serotype is selected based on its ability to cross the blood brain barrier. AAV9 and AAV10 have been shown to cross the blood brain barrier to transduce neurons and glia. In an aspect, an AAV vector is AAV2, AAV5, AAV6, AAV8, or AAV9. In some cases, an AAV vector is a chimera of at least two serotypes. In an aspect, an AAV vector is of serotypes AAV2, AAV5, and AAV9. In some cases, a chimeric AAV vector comprises rep and ITR sequences from AAV2 and a cap sequence from AAV5. In some cases, rep, cap, and ITR sequences can be mixed and matched from all the of the different AAV serotypes provided herein. In some cases, a suitable AAV vector can be further modified to encompass modifications such as in a capsid or rep protein. Modifications can also include deletions, insertions, mutations, and combinations thereof. In some cases, a modification to a vector is made to reduce immunogenicity to allow for repeated dosing. In some cases, a serotype of a vector that is utilized is changed when repeated dosing is performed to reduce and/or eliminate immunogenicity.

A vector can be used to deliver an engineered guide RNA provided herein and an additional polynucleotide targeting a therapeutic target, such as a second polynucleotide. In some cases, the vector comprises or encodes an additional RNA polynucleotide that associates with a second polynucleotide (e.g. an additional therapeutic target). Such vectors can be used to deliver multiplex therapeutics that simultaneously target multiple therapeutic targets, such as, in the case of Alzheimer′ and other neurodegenerative disease, amyloid precursor protein and an additional target implicated in the disease such as a Tau protein (e.g. a microtubule-associated protein Tau (MAPT) encoded from a MAPT gene), or an alpha-synuclein protein. Alternatively, or in addition, the additional target can be a further edit site on the polynucleotide encoding the amyloid precursor protein (e.g. on the same polynucleotide). The vector polynucleotide encoding the engineered guide RNA and the second vector polynucleotide encoding the additional RNA polynucleotide can be contiguous or not contiguous. When the first and second vector polynucleotides are contiguous with each other, they can be operatively linked to the same promoter sequence.

### Non-Viral Vector Approaches

In some cases, a vector may not be a viral vector. Non-viral methods can comprise naked delivery of compositions comprising polynucleotides and the like. In some cases, modifications provided herein can be incorporated into polynucleotides to increase stability and combat degradation when being delivered as naked polynucleotides. In other cases, a non-viral approach can harness use of nanoparticles, liposomes, and the like.

### Therapeutic Applications

The engineered guide RNAs provided herein can be used as therapeutics. In one aspect herein is a method of treating or preventing a condition comprising administering a therapeutic that facilitates an edit of an RNA at least partially encoding an amyloid precursor protein (APP), wherein the edited RNA encodes a BACE protease-resistant APP, as compared to an otherwise comparable APP produced from an otherwise comparable RNA that does not comprise the edit, as determined by in vitro assay comprising contacting the BACE protease-resistant APP and the otherwise comparable APP with a beta-secretase (e.g., BACE1, cathepsin B or Meprin beta), a γ-secretase, or a beta secretase and a γ-secretase. In some cases, the therapeutic directly facilitates the edit. In some cases, the therapeutic indirectly facilitates the edit. In some cases, the conditions can comprise a neurodegenerative condition.

Methods can include mRNA base editing of one or more targets associated with a neurodegenerative disease. A neurodegenerative disease can include Alzheimer, Parkinson's, or other conditions mediated via Abeta fragments, Tau, alpha-synuclein, or combinations thereof. Methods can include editing a portion of a polynucleotide encoding for an amyloid precursor protein (APP) such that an enzyme (such as a beta secretase (BACE) or a gamma secretase) cannot cut APP at the edited sites. Methods can include editing a portion of a polynucleotide encoding for an APP such that a fragment (such as an Abeta fragment) cannot form. Editing a portion of the polynucleotide encoding for APP can result in a substantial reduction in an amount of Abeta fragment formed. In some embodiments, the edited RNA or the BACE protease-resistant APP is generated in at least 5%, 8%, 10%, 15%, 20%, 30%, 40%, or 50% of the subjects administered the therapeutic in a clinical trial.

In some embodiments, the present disclosure also provides for combination therapies. For example, a therapeutic method may include RNA editing of target cleavage sites in APP using an engineered guide RNA of the present disclosure in combination with another therapeutic agent that may have the same target (APP) or different targets. Said additional therapeutic agents may include guide RNAs for editing or knockout of polynucleotides encoding other therapeutic targets (e.g., Tau, alpha-synuclein) or may include antibodies that clear unwanted metabolites of Beta-secretase (e.g., BACE1, cathepsin B or Meprin beta) cleavage of APP. Methods can include knockdown of APP, knockdown of Tau, knockdown of alpha-synuclein, or any combination thereof. Methods can include delivery of one or more anti-Abeta antibodies for clearing of Abeta fragments already present in a tissue space. When in combination with an mRNA base editing approach, dosing amounts of an antibody can be lower as compared to a dosing amount in the absence of an mRNA base editing approach. Methods can include delivery of one or more secretase inhibitors to reduce cleavage and thus Abeta fragment formation. When in combination with an mRNA base editing approach, dosing amounts of a secretase inhibitor can be lower as compared to a dosing amount in the absence of an mRNA base editing approach. Methods can include delivery or combination delivery of one or more compositions as described herein.

The therapeutic applications described in this invention can be used in a treatment to obtain a desired pharmacologic effect, physiologic effect, or any combination thereof. In some instances, a treatment can reverse an adverse effect attributable to the disease or condition. In some cases, the treatment can stabilize the disease or condition. In some cases, the treatment can delay progression of the disease or condition. In some instances, the treatment can cause regression of the disease or condition. In some instances, a treatment can at least partially prevent the occurrence of the disease, condition, or a symptom of any of these. In some embodiments, a treatment's effect can be measured. In some cases, measurements can be compared before and after administration of the composition. For example, a subject can have medical images prior to treatment compared to images after treatment to show cancer regression. In some instances, a subject can have an improved blood test result after treatment compared to a blood test before treatment. In some instances, measurements can be compared to a standard.

### Multiplexed Therapy

In some cases, the present disclosure encompasses multiplexed therapy, including multiplexed editing of multiple target RNAs, editing of multiple target sites within a target RNA, editing of RNA and knockdown, or any combination thereof. In some cases, use of vectors that contains multiple targeting guide RNAs can allow for simultaneous targeting of the Abeta generation and other proteins associated with neurodegeneration. Specifically, multiple unique, independent activities can be performed that regulate expression of complementary pathways affecting Alzheimer's, Tauopathies, Parkinson's, or neurodegeneration mediated via Tau, alpha-synuclein, and Abeta. Targeting more than one target RNA simultaneously may be important and the combination of Tau knockdown and editing of a cleavage site (e.g., a β-cleavage site) in APP may work in synergy. In some cases, use of mRNA base editing to knockdown (as opposed to just editing the cleavage site) expression of APP can be another approach for decreasing Abeta generation. As the compositions can be applied to gene expression knockdown, they could also include a combination of start-site editing to reduce expression, steric hinderance because the guide could block ribosomal activity, increased degradation of the targeted mRNA, or any combination thereof. The compositions and methods disclosed herein, thus, may suppress expression in an ADAR-dependent and ADAR-independent manner.

Both Abeta and Tau or SNCA are implicated in Alzheimer's disease initiation/progression. The compositions and methods disclosed herein can target both and, thus, may involve a multiplexed targeting approach. A multiplexed targeting approach can target 2, 3, 4, 5, 6, or more proteinopathies by independent mechanisms of action. For example, mRNA base editing using the engineered guide RNAs of the present disclosure can edit one or more cleavage sites of an APP protein preventing or substantially reducing Abeta fragment formation and mRNA base editing using the engineered guide RNAs of the present disclosure can knockdown Tau protein formation. In some cases, mRNA base editing using the guide RNAs of the present disclosure can edit one or more cleavage sites of an APP protein preventing or substantially reducing Abeta fragment formation and an additional therapeutic agent (e.g., an additional RNA polynucleotide, such as a siRNA, a shRNA, a miRNA, a piRNA, or an antisense oligonucleotide), which can knockdown Tau protein formation.

In some embodiments, a vector of the present disclosure may be a multiplex vector that contain multiple engineered guide polynucleotides targeting multiple target RNAs. In other cases, different engineered guide polynucleotide targeting different target RNAs can be maintained on different vectors. Vectors encoding for compositions that can (a) facilitate an edit to a cleavage site of a protein target (e.g., via association with an RNA editing protein such as ADAR), (b) reduce an amount of a protein target produced, (c) regulate the activity of a protein target produced, or (d) a combination thereof. A vector or a multiplex vector or vectors can be formulated in unit dose form. A multiplex vector can be configured to modulate more than one protein target implicated in a neurodegenerative disease. A vector can reduce an amount of the protein target by (i) performing an edit to a sequence that encodes for the protein, (ii) performing an edit to a sequence that does not encode for the protein, (iii) sterically hindering a promoter region associated with the protein target, or (iv) any combination thereof. The protein target can comprise amyloid beta, Tau, alpha-synuclein, or any combination thereof.

In some embodiments, a target RNA subjected to a multiplex targeting or edit can have at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 76-fold, at least 77-fold, at least 78-fold, at least 79-fold, at least 80-fold, at least 81-fold, at least 82-fold, at least 83-fold, at least 84-fold, at least 85-fold, at least 86-fold, at least 87-fold, at least 88-fold, at least 89-fold, at least 90-fold, at least 91-fold, at least 92-fold, at least 93-fold, at least 94-fold, at least 95-fold, at least 96-fold, at least 97-fold, at least 98-fold, at least 99-fold, at least 100-fold, 1-20-fold, 2-21-fold, 3-22-fold, 4-23-fold, 5-24-fold, 6-25-fold, 7-26-fold, 8-27-fold, 9-28-fold, 10-29-fold, 11-30-fold, 12-31-fold, 13-32-fold, 14-33-fold, 15-34-fold, 16-35-fold, 17-36-fold, 18-37-fold, 19-38-fold, 20-39-fold, 21-40-fold, 22-41-fold, 23-42-fold, 24-43-fold, 25-44-fold, 26-45-fold, 27-46-fold, 28-47-fold, 29-48-fold, 30-49-fold, 31-50-fold, 32-51-fold, 33-52-fold, 34-53-fold, 35-54-fold, 36-55-fold, 37-56-fold, 38-57-fold, 39-58-fold, 40-59-fold, 41-60-fold, 42-61-fold, 43-62-fold, 44-63-fold, 45-64-fold, 46-65-fold, 47-66-fold, 48-67-fold, 49-68-fold, 50-69-fold, 51-70-fold, 52-71-fold, 53-72-fold, 54-73-fold, 55-74-fold, 56-75-fold, 57-76-fold, 58-77-fold, 59-78-fold, 60-79-fold, 61-80-fold, 62-81-fold, 63-82-fold, 64-83-fold, 65-84-fold, 66-85-fold, 67-86-fold, 68-87-fold, 69-88-fold, 70-89-fold, 71-90-fold, 72-91-fold, 73-92-fold, 74-93-fold, 75-94-fold, 76-95-fold, 77-96-fold, 78-97-fold, 79-98-fold, 80-99-fold, or 81-100-fold decrease in the protein level of Abeta 40, 42, or both, as compared to that generated upon beta secretase (e.g., BACE1, cathepsin B or Meprin beta) cleavage of unedited APP.

A composition with multiplexed targeting of target RNAs can be at least at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 76-fold, at least 77-fold, at least 78-fold, at least 79-fold, at least 80-fold, at least 81-fold, at least 82-fold, at least 83-fold, at least 84-fold, at least 85-fold, at least 86-fold, at least 87-fold, at least 88-fold, at least 89-fold, at least 90-fold, at least 91-fold, at least 92-fold, at least 93-fold, at least 94-fold, at least 95-fold, at least 96-fold, at least 97-fold, at least 98-fold, at least 99-fold, at least 100-fold more effective in halting or inhibiting Alzheimer's disease progression or development.

In some cases, polynucleotide base editing can be used in conjunction with an additional method of knocking down gene expression of either the same gene targeted by the polynucleotide base editing or an additional gene implicated in a disease, such as a neurodegenerative disease. For example, mRNA base editing (e.g. using the engineered guide RNAs disclosed herein) can be used in conjunction with an RNA polynucleotide that associates with an mRNA sequence to minimize expression of a targeted gene. Examples of such RNA polynucleotides capable of minimizing expression of a targeted gene include small interfering RNA (siRNA), short hairpin RNA (shRNA), microRNA (miRNA), piwi-interacting RNA (piRNA), or an anti-sense oligonucleotide (ASO). In some cases, the ASO comprises a variant oligonucleotide structure that stabilizes the oligonucleotide and/or minimizes nuclease activity on the nucleotide. Examples of such variants oligonucleotides include morpholino oligomers. Thus, the present disclosure provides for compositions of engineered guides RNAs in combination with one or more additional therapeutic agents selected from small interfering RNA (siRNA), short hairpin RNA (shRNA), microRNA (miRNA), piwi-interacting RNA (piRNA), and an antisense oligonucleotide (ASO).

Abeta oligomers can initiate pathogenesis in Alzheimer's disease, however, their suppression cannot be sufficient to reduce disease progression. Targeting insoluble Abeta and Abeta aggregates can ignore the toxicity of soluble Abeta and/or C-terminal fragments, which can be highly toxic as well. Expectations of an effect with only Abeta reduction can ignore other identified pathogenic drivers (e.g., p-Tau, alpha-synuclein).

Genetic/neuropathologic evidence can link amyloid-beta aggregation (Abeta/amyloid-beta plaques) and Tau-p tangles with AD. Abeta can be derived from sequential proteolysis of amyloid precursor protein (APP) as variable-length fragments. Longer-length Abeta fragments (greater than 42 AA in length) can aggregate, forming plaques. Abeta fragments can drive Tau phosphorylation leading to aberrant Tau aggregation and gain-of-toxicity (synapse loss, or neurotoxicity). In an embodiment, a level of a beta amyloid plaque can be determined, for example using an in vivo diagnostic such as a PET scan, blood draw, and/or spinal tap. In another embodiment, a metabolite, including amyloid beta 40 (Abeta 40) and amyloid beta 42 (Abeta 42), can be measured.

Therefore, a multi-targeted combination therapy, such as described in the compositions and methods herein, can provide the necessary disease modification in Alzheimer's disease. A composition, including the disclosed engineered guide RNAs, as described herein may be administered with an additional therapeutic agent. An additional therapeutic agent can be a 5-HT 6 antagonist, 5-HT2A inverse agonist, an AB42 lowering agent, an acetylcholinesterase inhibitor, an alpha secretase enhancer, an alpha-1 adrenoreceptor antagonist, an alpha-2 adrenergic agonist, an angiotensin II receptor blocker, an angiotensin receptor blocker, an anti-amyloid antibody, an anti-aggregation agent, an anti-amyloid immunotherapy, an anti-inflammatory agent, an antimalarial glial cell modulator, an antioxidant, an anti-Tau antibody, an anti-Tau immunotherapy, a BACE inhibitor, a beta-2 adrenergic receptor agonist, an arginase inhibitor, a beta-HSD1 inhibitor, a calcium channel blocker, a cannabinoid, a CB1 or CB2 endocannabinoid receptor agonist, a cholesterol lowering agent, a D2 receptor agonist, a dopamine-norepinephrine reuptake inhibitor, a FLNA inhibitor, a gamma secretase inhibitor, a GABA receptor modulator, a glucagon-like peptide 1 receptor agonist, a glutamate modulator, a glutamate receptor antagonist, a glycine transporter 1 inhibitor, a gonadotropin-releasing hormone receptor agonist, a GSK-3B inhibitor, a hepatocyte growth factor, a histone deacetylase inhibitor, a IgG1-Fc-GAIM fusion protein, an ion channel modulator, an iron chelating agent, a leukotriene receptor antagonist, a MAPT RNA inhibitor, a mast cell stabilizer, a melatonin receptor agonist, a microtubule protein modulator, a mitochondrial ATP synthase inhibitor, a monoamine oxidase B inhibitor, a muscarinic agonist, a nicotinic acetylcholine receptor agonist, an NMDA antagonist, an NMDA receptor modulator, a non-hormonal estrogen receptor B agonist, a non-nucleoside reverse transcriptase inhibitor, a nonsteroidal anti-inflammatory agent, an omega-3 fatty acid, a P38 MAPK inhibitor, a P75 neurotrophin receptor ligand, a PDE 5 inhibitor, a PDE-3 inhibitor, a PDE4D inhibitor, a positive allosteric modulator of GABA-A receptors, a PPAR-gamma agonist, a protein kinase C modulator, a RIPK1 inhibitor, a selective inhibitor of APP production, a selective norepinephrine reuptake inhibitor, a selective serotonin reuptake inhibitor, a selective tyrosine kinase inhibitor, a SGLT2 inhibitor, a SIGLEC-3 inhibitor, a sigma-1 receptor agonist, a sigma-2 receptor antagonist, a stem cell therapy, an SV2A modulator, a synthetic granulocyte colony stimulator, synthetic thiamine, a Tau protein aggregation inhibitor, a telomerase reverse transcriptase vaccine, a thrombin inhibitor, a transport protein ABCC1 activator, a TREM2 inhibitor, or any combination thereof. An additional therapeutic agent can be AADvac1, AAVrh.10hAPOE2, ABBV-8E12, ABvac40, AD-35, aducanumab, AGB101, AL002, AL003, allopregnanolone, amlopidine, AMX0035, ANAVEX 2-73, APH-1105, AR1001, AstroStem, atorvastatin, AVP-786, AXS-05, BAC, benfotiamine, BHV4157, BI425809, BIIB092, BIIP06, bioactive dietary polyphenol preparation, BPN14770, brexpiprazole, byrostatin, CAD106, candesartan, CERE-110, cilostazol, CKD-355, CNP520, COR388, crenezumab, cromolyn, CT1812, curcumin, dabigatran, DAOI, dapagliflozin, deferiprone, DHA, DHP1401, DNL747, dronabinol, efavirenz, elderberry juice, elenbecestat, escitalopram, formoterol, gantenerumab, Ginkgo biloba, grapeseed extract, GRF6019, guanfacine, GV1001, hUCB-MSCs, ibuprofen, icosapent ethyl, ID1201, insulin aspart, insulin glulisine, IONIS MAPTRx, J147, JNJ-63733657, lemborexant, leuprolide acetate depot, levetiracetam, liraglutide, lithium, LM11A-31-BHS, losartan, L-serine, Lu AF20513, LY3002813, LY3303560, LY3372993, masitinib, methylene blue, methylphenidate, mirtazapine, ML-4334, MLC901, montelukast, MP-101, nabilone, NDX-1017, neflamapimod, nicotinamide, nicotine, nilotinib, NPT08, octagam 10%, octohydroaminoacridine succinate, omega-3 PUFA, perindopril, pimavanserin, piromelatine, posiphen, prazosin, PTI-125, rasagiline, riluzole, RO7105705, RPh201, sagramostim, salsalate, S-equol, solanezumab, SUVN-502, telmisartan, TEP, THN201, TPI-287, traneurocin, TRx0237, UB-311, valacyclovir, venlafaxine hMSCs (human mesenchymal stem cells), vorinostat, xanamem, zolpidem, or any combination thereof. The additional therapeutic agent can be administered concurrently, consecutively, or in any order.

An mRNA base editing approach using an engineered polynucleotide, such as guide RNA, of the present disclosure can be combined with an antibody-based approach (such as anti-Abeta antibodies). Disadvantages to employing an antibody-based approach alone can include low/inefficient transfer across the blood-brain-barrier and development of ARIA (neuroinflammation) in patients treated with these therapies constrain the therapeutic dose. It is likely that more than one Abeta species (including soluble Abeta) contribute to disease progression. Thus, multiple different antibodies to the different species can be needed. Antibodies that may be combined with the engineered guide RNAs disclosed herein for combination treatment of a subject in need thereof can include bapineuzumab, solanezumab, gantenerumab, crenezumab, ponezumab, aducanumab, BAN2401, or any combination thereof.

An mRNA base editing approach using the engineered polynucleotides of the present disclosure can be combined with a secretase inhibitor approach (such as a beta secretase and γ-secretase inhibitors). Both enzymes appear to have proteolytic activity necessary for maintenance of synaptic function/neuronal health and thus severely or completely reducing function can led to poor long-term outcomes. Utilizing a combined approach of an mRNA base editing and secretase inhibitor approach can permit reducing dosing of the secretase inhibitor as compared to a solitary approach of delivering the secretase inhibitor alone. Inhibitors that may be combined with the engineered guide RNAs disclosed herein for combination treatment of a subject in need thereof can include verubecestat, atabecestat, lanabecestat, elenbecestat, umibecestat, avagacestat, semagacestat, or any combination thereof.

Compositions and methods as described herein can provide improvements over existing technologies and therapeutics, such as secretase inhibitors. Non-limiting and exemplary benefits and advantages of the compositions and methods as described herein can include (a) being capable of simultaneously targeting any and/or all forms of Abeta fragments (including those of varying length, and those that can be soluble or aggregated) and (b) without altering (i) the endogenous APP function, (ii) the endogenous β/γ-secretase function, or (iii) inflammation (e.g., ARIA).

### Pharmaceutical Compositions

Compositions can include any editing entity described herein. A pharmaceutical composition can comprise a first active ingredient. The first active ingredient can comprise a viral vector as described herein, a non-naturally occurring RNA as described herein, or a nucleic acid as described herein. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient—such as to provide multiplex targeting of related proteinopathies.

A composition described herein can compromise an excipient. An excipient can be added to a stem cell or can be co-isolated with the stem cell from its source. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a trigylceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof.

Compositions and methods disclosed herein can include multiplexed targeting via an mRNA base editing approach. For example, the present disclosure provides for multiplexed vectors for multiplexed targeting of Abeta generation and one or more additional proteins associated with a neurodegeneration disease or condition, such as Tau knockdown or alpha-synuclein knockdown. These multiplexed vectors may encode for engineered guide RNAs targeting APP (to affect Abeta generation) and one or more additional engineered guide RNAs or other therapeutic agents disclosed herein targeting one or more additional proteins associated with a neurodegeneration disease or condition. Compositions can perform multiple unique, independent bioactivities that regulate expression of complementary pathways affecting neurodegenerative conditions or Tau-pathologies (such as Alzheimer's, Parkinson's). Complementary pathways can include pathologies mediated via Tau, alpha-synuclein, and Abeta. In some cases, compositions can independently target more than one target RNA polynucleotide simultaneously such as a combination of Tau knockdown and APP editing. Compositions can include 2, 3, 4, 5, 6 vectors or more, independently targeting complementary pathways. Multiplexed targeting can result in additive therapeutic effects in subjects administered multiplexed vectors. Multiplexed targeting can provide a synergistic therapeutic effect in subjects administered multiplexed vectors, providing a greater therapeutic outcome than an individual targeting scheme.

Compositions can include mRNA base editing to (i) edit a cleavage site (such as to reduce or prevent Abeta fragment formation), (ii) knockdown protein expression (such as APP), or (iii) a combination thereof. Compositions can be designed to edit a base, edit a start-site (such as to reduce expression), create steric hindrance (such as a guide RNA that can block ribosomal activity), increase degradation of a targeted mRNA, or any combination thereof. The compositions and methods disclosed herein, thus, may suppress expression in an ADAR-dependent and -independent manner.

Editing can include editing of a target site (such as a BACE cleavage site) to prevent or substantially reduce cleavage of a protein by an enzyme. Editing can include knockdown of a protein (such as a protein implicated as a proteinopathy in a neurodegenerative disease) achieved by (i) start site editing (such as ATG), (ii) exon skipping (such as an exon that can contain the start site), (iii) blocking a region from promoter accessibility (such as an antisense-oligonucleotide (ASO)-based approach), or (iv) any combination thereof.

Compositions of the present disclosure can include an engineered guide RNA for editing a nucleotide in a target RNA polynucleotide sequence. Compositions can employ editing in an ADAR dependent or ADAR independent manner. Compositions can comprise a recruiting domain that recruits an RNA editing entity.

Compositions and methods described herein can allow for multiplexed targeting of both APP and Tau or SNCA mRNA to halt or prevent Alzheimer's disease progression. Delivery of a vector with multiple engineered polynucleotides targeting different target RNAs can allow for simultaneous or multiplexed targeting of the target RNAs. In some cases, delivery of vectors, each with an engineered polynucleotide targeting a specific target RNAs, can also allow for simultaneous or multiplexed targeting of the target RNAs.

An mRNA base editing approach can edit one or more BACE cleavage sites on APP, and thereby can mitigate toxic Abeta fragment formation or accumulation while maintaining normal or non-diseased APP/BACE function. A knockdown approach can simultaneously target Tau production to reduce Tau-p accumulation.

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

A disease or condition may include a neurodegenerative disease. A disease can include a disease associated with one or more proteinopathies (such as APP, Tau, or alpha-synuclein). A neurodegenerative disease can include Alzheimer's disease, amyotrophic lateral sclerosis, ataxia-telangiectasia, autosomal dominant cerebellar ataxia, autosomal recessive spastic ataxia of Chrlevoix-Saguenay, Baggio-Yoshinari syndrome, Batten disease, Cohen-Gibson syndrome, Corticobasal degeneration (CBD), corticobasal syndrome, Creutzfeldt-Jakob disease, dementia, fatal insomnia, fragile X-associated tremor/ataxia syndrome, Friedreich's ataxia, frontotemporal dementia, Fronto-temporal dementia with Parkinsonism linked to Tau mutations on chromosome 17 (FTDP-17T), heredetitary motor and sensory neuropathy with proximal dominance, Huntington's disease (HD), infantile refsum disease, JUNQ and IPOD, Kufor-Rakeb syndrome, Kufs disease, Lewy body disease, Lewy body variant of Alzheimer's disease (LBVAD), locomotor ataxia, Lyme disease, Machado-Joseph disease, motor neurone diseases (MND), multiple system atrophy, neuroacanthocytosis, Niemann-Pick disease, Parkinson's disease (PD), Parkinson's disease with dementia (PDD), Pick's disease (PiD), or Progressive supranuclear palsy (PSP), pontocerebellar hypoplasia, prion disease, Refsum disease, Sandhoff disease, Shy-Drager syndrome, spinal muscular atrophy, spinocerebellar ataxia (SCA), spinocerrebellar ataxia, subacute combined degeneration of spinal cord, subacute sclerosing panencephalitis, tabes dorsalis, Tay-Sachs disease, toxic encephalapothay, toxic leukoencephalopathy, transneuronal degeneration, Wobbly Hedgehog syndrome or any combination thereof. A disease or condition can include a disease associated with Tau plaque formation including Alzheimer's disease, corticobasal degeneration, dementia pugilistica (chronic traumatic encephalopathy), frontotemporal dementia, frontotemporal lobar degeneration, gangliocytoma, ganglioglioma, lytico-bodig disease, meningioangiomatosis, Pick's disease, postencephalitic parkinsonism, primary age-related Tauopathy, progressive supranuclear palsy, Tauopathy, or any combination thereof. A disease or condition can include a disease associated with alpha-synuclein formation including Alzheimer's disease, dementia with Lewy bodies, Lewy body disease, multiple system atrophy, Parkinson's disease, or any combination thereof. A disease or condition can include a disease associated with Abeta fragment formation including Alzheimer's disease, cerebral amyloid angiopathy, inclusion body mitosis, Lew body dementia, or any combination thereof. A disease (or condition) can also include traumatic brain injury, Down's syndrome, a cancer, Fragile X Syndrome, autism, amyotrophic lateral sclerosis, multiple sclerosis, Lesch-Nyhan disease, a metabolic disorder, or any combination thereof. The disease can include a neurodegenerative disease.

The compositions provided herein can be utilized in methods provided herein. Any of the provided compositions provided herein can be utilized in methods provided herein. In some cases, a method comprises at least partially preventing, reducing, ameliorating, and/or treating a disease or condition, or a symptom of a disease or condition. In an embodiment, a composition can be utilized to reduce a level of a beta amyloid plaque in a subject, evaluated utilizing an in vivo diagnostic. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). In an embodiment, a subject is over the age of 40, 50, 60, or 70.

### Administration Routes and Dosing

Compositions described herein can employ an AAV (IV/CNS) vector for delivery to a subject. AAV vector delivery can achieve long-term benefits with single dose and can provide opportunity for multiplexed targeting. Methods can include identifying AAV serotypes that can promote central neuronal tropism and biodistribution with CNS/IV dosing.

In some cases, an administration can refer to methods that can be used to enable delivery of compounds or compositions to the desired site of biological action. Delivery can include direct application to the central nervous system (CNS). Delivery can include one that is permissive to cross the blood brain barrier. Delivery can include direct application to the affect tissue or region of the body. Delivery can include intracranial injection. Delivery can include a parenchymal injection, an intra-thecal injection, an intra-ventricular injection, or an intra-cisternal injection. A composition provided herein can be administered by any method. A method of administration can be by inhalation, intraarterial injection, intracerebroventricular injection, intracisternal injection, intramuscular injection, infraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some cases, administration is by parenchymal injection, intra-thecal injection, intra-ventricular injection, intra-cisternal injection, intravenous injection, or intranasal administration or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). A medical professional can administer the composition. In some cases, a cosmetic professional can administer the composition.

Administration or application of a composition, including any of the engineered guide RNAs, multiplexed vectors, or combination therapies, disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. A treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of a composition, including any of the engineered guide RNAs, multiplexed vectors, or combination therapies, disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

In some cases, an administration of any composition provided herein, including pharmaceutical compositions can be in an effective amount, for example to reduce a symptom of a disease or condition and/or to reduce a disease or condition. An effective amount can be sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above.

Administration or application of the compositions disclosed herein, including any of the engineered guide RNAs, multiplexed vectors, or combination therapies, can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

A composition of the present disclosure, including any of the engineered guide RNAs, multiplexed vectors, or combination therapies, can be administered/applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

Vectors of the disclosure can be administered at any suitable dose to a subject. Suitable doses can be at least about 5×10⁷ to 50×10¹³ genome copies/mL. In some cases, suitable doses can be at least about 5×10⁷, 6×10⁷, 7×10⁷, 8×10⁷, 9×10⁷, 10×10⁷, 11×10⁷, 15×10⁷, 20×10⁷, 25×10⁷, 30×10⁷ or 50×10⁷ genome copies/mL. In some embodiments, suitable doses can be about 5×10⁷ to 6×10⁷, 5.9×10⁷ to 7×10⁷, 6.9×10⁷ to 8×10⁷, 7.9×10⁷ to 9×10⁷, 8.9×10⁷ to 10×10⁷, 9.9×10⁷ to 11×10⁷, 10.9×10⁷ to 15×10⁷, 14.9×10⁷ to 20×10⁷, 19.9×10⁷ to 25×10⁷, 24.9×10⁷ to 30×10⁷, 29.9×10⁷ to 50×10⁷, or 49.9×10⁷ to 100×10⁷ genome copies/mL. In some cases, suitable doses can be about 5×10⁷ to 10×10⁷, 9.9×10⁷ to 25×10⁷, or 24.9×10⁷ to 50×10⁷ genome copies/mL. In some cases, suitable doses can be at least about 5×10⁸, 6×10⁸, 7×10⁸, 8×10⁸, 9×10⁸, 10×10⁸, 11×10⁸, 15×10⁸, 20×10⁸, 25×10⁸, 30×10⁸ or 50×10⁸ genome copies/mL. In some embodiments, suitable doses can be about 5×10⁸ to 6×10⁸, 5.9×10⁸ to 7×10⁸, 6.9×10⁸ to 8×10⁸, 7.9 to 9×10⁸, 8.9×10⁸ to 10×10⁸, 9.9×10⁸ to 11×10⁸, 10.9×10⁸ to 15×10⁸, 14.9×10⁸ to 20×10⁸, 19.9×10⁸ to 25×10⁸, 24.9×10⁸ to 30×10⁸, 29.9×10⁸ to 50×10⁸, or 49.9×10⁸ to 100×10⁸ genome copies/mL. In some cases, suitable doses can be about 5×10⁸ to 10×10⁸, 9.9×10⁸ to 25×10⁸, or 24.9×10⁸ to 50×10⁸ genome copies/mL. In some cases, suitable doses can be at least about 5×10⁹, 6×10⁹, 7×10⁹, 8×10⁹, 9×10⁹, 10×10⁹, 11×10⁹, 15×10⁹, 20×10⁹, 25×10⁹, 30×10⁹ or 50×10⁹ genome copies/mL. In some embodiments, suitable doses can be about 5×10⁹ to 6× 10⁹, 5.9×10⁹ to 7×10⁹, 6.9×10⁹ to 8×10⁹, 7.9×10⁹ to 9×10⁹, 8.9×10⁹ to 10×10⁹, 9.9×10⁹ to 11×10⁹, 10.9×10⁹ to 15×10⁹, 14.9×10⁹ to 20×10⁹, 19.9×10⁹ to 25×10⁹, 24.9×10⁹ to 30×10⁹, 29.9×10⁹ to 50×10⁹, or 49.9×10⁹ to 100×10⁹ genome copies/mL. In some cases, suitable doses can be about 5×10⁹ to 10×10⁹, 10×10⁹ to 25×10⁹, or 25×10⁹ to 50×10⁹ genome copies/mL. In some cases, suitable doses can be at least about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰ or 50×10¹⁰ genome copies/mL. In some embodiments, suitable doses can be about 5×10¹⁰ to 6×10¹⁰, 5.9×10¹⁰ to 7×10¹⁰, 6.9 to 8×10¹⁰, 7.9×10¹⁰ to 9×10¹⁰, 8.9×10¹⁰ to 10×10¹⁰, 9.9×10¹⁰ to 11×10¹⁰, 10.9×10¹⁰ to 15×10¹⁰, 14.9×10¹⁰ to 20×10¹⁰, 19.9×10¹⁰ to 25×10¹⁰, 24.9×10¹⁰ to 30×10¹⁰, 29.9×10¹⁰ to 50×10¹⁰, or 49.9×10¹⁰ to 100×10¹⁰ genome copies/mL. In some cases, suitable doses can be about 5×10¹⁰ to 10×10¹⁰, 10×10¹⁰ to 25×10¹⁰, or 25×10¹⁰ to 50×10¹⁰ genome copies/mL. In some cases, suitable doses can be at least about 5×10¹¹, 6×10¹¹, 7×10¹¹, 8×10¹¹, 9×10¹¹, 10×10¹¹, 11×10¹¹, 15×10¹¹, 20×10¹¹, 25×10¹¹, 30×10¹¹ or 50×10¹¹ genome copies/mL. In some embodiments, suitable doses can be about 5×10¹¹ to 6×10¹¹, 5.9×10¹¹ to 7×10¹¹, 6.9 to 8×10¹¹, 7.9×10¹¹ to 9×10¹¹, 8.9×10¹¹ to 10×10¹¹, 9.9×10¹¹ to 11×10¹¹, 10.9×10¹¹ to 15×10¹¹, 14.9×10¹¹ to 20×10¹¹, 19.9×10¹¹ to 25×10¹¹, 24.9×10¹¹ to 30×10¹¹, 29.9×10¹¹ to 50×10¹¹, or 49.9×10¹¹ to 100×10¹¹ genome copies/mL. In some cases, suitable doses can be about 5×10¹¹ to 10×10¹¹, 10×10¹¹ to 25×10¹¹, or 25×10¹¹ to 50×10¹¹ genome copies/mL. In some cases, suitable doses can be at least about 5×10¹², 6×10¹², 7×10¹², 8×10¹², 9×10¹², 10×10¹², 11×10¹², 15×10¹², 20×10¹², 25×10¹²30×10¹² or 50×10¹² genome copies/mL. In some embodiments, suitable doses can be about 5×10¹² to 6×10¹², 5.9×10¹² to 7×10¹², 6.9 to 8×10¹², 7.9×10¹² to 9×10¹², 8.9×10¹² to 10×10¹², 9.9×10¹² to 12×10¹², 10.9×10¹² to 15×10¹², 14.9×10¹² to 20×10¹², 19.9×10¹² to 25×10¹², 24.9×10¹² to 30×10¹²29.9×10¹² to 50×10¹², or 49.9×10¹² to 100×10¹² genome copies/mL. In some cases, suitable doses can be about 5×10¹² to 10×10¹², 9.9×10¹² to 25×10¹², or 24.9×10¹² to 50×10¹² genome copies/mL. In some cases, suitable doses can be at least about 5×10¹³, 6×10¹³, 7×10¹³, 8× 10¹³, 9×10¹³, 10×10¹³11×10¹³, 15×10¹³, 20×10¹³, 25×10¹³, 30×10¹³ or 50×10¹³ genome copies/mL. In some embodiments, suitable doses can be about 5×10¹³ to 6×10¹³, 5.9×10¹³ to 7×10¹³, 6.9 to 8×10¹³, 7.9×10¹³ to 9×10¹³, 8.9×10¹³ to 10×10¹³, 9.9×10¹³ to 13×10¹³, 10.9×10¹³ to 15×10¹³, 14.9×10¹³ to 20×10¹³, 19.9×10¹³ to 25×10¹³, 24.9×10¹³ to 30×10¹³, 29.9×10¹³ to 50×10¹³, or 49.9×10¹³ to 100×10¹³ genome copies/mL. In some cases, suitable doses can be about 5×10¹³ to 10×10¹³, 9.9×10¹³ to 25×10¹³, or 24.9×10¹³ to 50×10¹³ genome copies/mL. In some cases, suitable doses can be at least about 5× 10¹³, 6×10¹³, 7×10¹³, 8× 10¹³, 9×10¹³, 10×10¹³, 11×10¹³, 15×10¹³, 20×10¹³, 25×10¹³, 30×10¹³ or 50×10¹³ genome copies/mL. In some embodiments, suitable doses can be about 5×10¹³ to 6×10¹³, 5.9×10¹³ to 7×10¹³, 6.9 to 8×10¹³, 7.9×10¹³ to 9×10¹³, 8.9×10¹³ to 10×10¹³, 9.9×10¹³ to 13×10¹³, 10.9×10¹³ to 15×10¹³, 14.9×10¹³ to 20×10¹³, 19.9×10¹³ to 25×10¹³, 24.9×10¹³ to 30×10¹³, 29.9×10¹³ to 50×10¹³, or 49.9×10¹³ to 100×10¹³ genome copies/mL. In some cases, suitable doses can be about 5×10¹³ to 10×10¹³, 9.9×10¹³ to 25×10¹³, or 24.9×10¹³ to 50×10¹³ genome copies/mL.

In some cases, the dose of virus particles administered to the individual can be any at least about 1×10⁷ to about 1×10¹³ genome copies/kg body weight. In some embodiments, the dose of virus particles administered to the individual can be 1×10⁷, 2×10⁷, 3×10⁷, 4×10⁷, 5×10⁷, 6×10⁷, 7×10⁷, 8×10⁷, or 9×10⁷ genome copies/kg body weight. In some embodiments, the dose of virus particles administered to the individual can be 1×10⁸, 2×10⁸, 3×10⁸, 4×10⁸, 5×10⁸, 6×10⁸, 7×10⁸, 8×10⁸, or 9×10⁸ genome copies/kg body weight. In some embodiments, the dose of virus particles administered to the individual can be 1×10⁹, 2×10⁹, 3×10⁹, 4×10⁹, 5×10⁹, 6×10⁹, 7×10⁹, 8×10⁹, or 9×10⁹ genome copies/kg body weight. In some embodiments, the dose of virus particles administered to the individual can be 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ genome copies/kg body weight. In some embodiments, the dose of virus particles administered to the individual can be 1×10¹¹, 2×10¹¹, 3×10¹¹, 4×10¹¹, 5× 10¹¹, 6× 10¹¹, 7×10¹¹, 8×10¹¹, or 9×10¹¹ genome copies/kg body weight. In some embodiments, the dose of virus particles administered to the individual can be 1×10¹², 2×10¹², 3×10¹², 4×10¹², 5× 10¹², 6× 10¹², 7× 10¹², 8×10¹², or 9×10¹² genome copies/kg body weight. In some embodiments, the dose of virus particles administered to the individual can be 1× 10¹³, 2× 10¹³, 3 × 10¹³, 4×10¹³, 5×10¹³, 6×10¹³, 7×10¹³8× 10¹³, or 9×10¹³ genome copies/kg body weight.

### Methods and Systems for Diagnosing a Disease or Monitoring a Disease Progression

Doctors will use medical history, physical e×am, neurological e×am, mental status test, genetic test, and brain imaging to diagnose Alzheimer's disease. Medical history consultation can comprise e×amining whether there are current or past illness or if family members may have Alzheimer's disease. Physical e×am can help identify medical issues causing dementia-like symptoms. Physical e×am can comprise e×amining diet, nutrition, alcohol use, medications, blood pressure, temperature, pulse, heart and lung functions, or other health conditions. Physical e×am can also comprise blood and urine test. Neurological e×am can evaluate if a patient has other brain disorders other than Alzheimer's disease. Neurological e×am can comprise testing refle×es, coordination, muscle tone/strength, eye movement, speech, or sensation. Neurological e×am can also comprise brain imaging study including but not limited to Magnetic resonance imaging (MRI), computerized tomography (CT), or Positron emission tomography (PET). Mental status test can evaluate memory, problem-solving ability, or other cognitive abilities. Mental status test can comprise examining self-awareness, temporal or spatial awareness, memory, calculation ability, or others cognitive abilities. Mental status test can also comprise Mini-Mental State Exam (MMSE), the Mini-Cog test, FDA-approved computerized tests, mood assessment, or others. FDA-approved computerized tests can comprise the Cantab Mobile, Cognigram, Cognivue, Cognision and Automated Neuropsychological Assessment Metrics (ANAM) devices. Genetic testing can comprise testing APP, PSEN-1, PSEN-2, or apoE4. Other risk genes of Alzheimer's disease include ABCA7, CLU, CR1, PICALM, PLD3, TREM2, or SORL1. With all the information listed above, a doctor can determine if a patient has “possible Alzheimer's dementia” (dementia may be due to another cause), “probable Alzheimer's dementia” (no other cause for dementia can be found), or some other problems.

### Kits

Any of the compositions described herein may be comprised in a kit. In a non-limiting example, a vector, a polynucleotide, a peptide, reagents to generate polynucleotides provided herein, and any combination thereof may be comprised in a kit. In some cases, kit components are provided in suitable container means.

Kits may comprise a suitably aliquoted composition. The components of the kits may be packaged either in aqueous media or in lyophilized form. The container means of the kits will generally include at least one vial, test tube, flask, bottle, syringe, or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

In some embodiments, a kit can comprise an engineered guide RNA, a precursor engineered guide RNA, a vector comprising the engineered guide RNA or the precursor engineered guide RNA, or a nucleic acid of the engineered guide RNA or the precursor engineered guide RNA, or a pharmaceutical composition and a container. In some instances, a container can be plastic, glass, metal, or any combination thereof.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

### EXAMPLES

### Example 1: Treatment of Alzheimer's Disease

This example describes treatment of Alzheimer's disease using the engineered guide RNAs of the present disclosure.

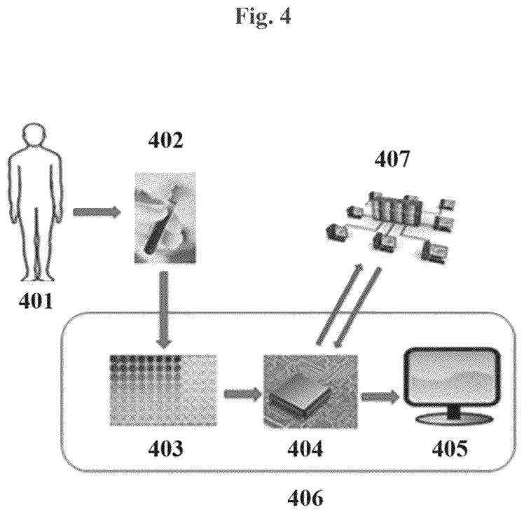

A subject is diagnosed with Alzheimer's disease. FIG. 4 shows an example of such diagnosis.

The central idea is to mutagenize the beta-cleavage sites and the amino acids around them RNA editing. This approach has the dual advantages of: 1) directly modulating a driver event in the disease by diminishing the substrate preferences of beta-secretase (primarily BACE1 but also others); and 2) not interfering normal APP functions but leaving the endogenous APP expression largely unaffected. BACE1 substrate preferences are shown below in TABLE 8.

TABLE 8
Substrate Specificity of BACE1 (organized by the allotment of amino acids on each side of the
cleavage bond at each position of the protein substrate (numbered from amino to carboxy terminus:
P4, P3, P2, P1, P1′, P2′, P3′, P4′; A > B A is more specific to BACE-1 than B;
A = B, A is as specific to BACE-1 as B)

| Cleavage | | |
|---|---|---|
| site | Terminus | Substrate Specificity of BACE-1 |
| P4 | N- | E > Q > D > N > M > G > L = T = H = P > R > V = W > F > A = S > |
| | | I > Y |
| P3 | N- | I > V > L > E > H > M = A = T = P > K > S > F > D > Q > G = Y |
| P2 | N- | D > N > M > F > Y = L > S = E > A > Q > K > G |
| P1 | N- | L > F > M > Y >>> T > S > D > G > N > H > A |
| P1′ | C- | M > E > Q > A > D > S >>> Y > L > T > V > I > F > R > K > G > |
| | | N > W |
| P2′ | C- | V > I > A > E > F > L > T > M > Y > S >> G > Q > N = D = W = K |
| P3′ | C- | L > V > W > I > T > D > E > F > Y > M > R > K > A > G > S > |
| | | Q > N > H > P |
| P4′ | C- | D > E > W > F = Y = M > V > L > I > T > A > Q > G = S >> R > H > |
| | | K > N |

The subject is prescribed a dosing regimen of a pharmaceutical composition. The pharmaceutical composition comprises an engineered polynucleotide or at least two engineered polynucleotides (e.g., any engineered guide RNA of the present disclosure) that edits a secretase cleavage site. The secretase cleavage site is a BACE secretase cleavage site. The cleavage site is the beta- or the beta′-cleavage site in APP (e.g., any of the APP isoforms disclosed herein). The pharmaceutical composition is administered to the subject by direct injection to cranial tissue, ICM injection, or ICV injection. The subject is a human or non-human animal. An amount of Abeta fragment formation following treatment with the engineered guide RNA is less (e.g., at least about 4-fold less) than an amount of Abeta fragment formation following treatment with a secretase inhibitor. Upon administration of the engineered guide RNA to the subject—for example but not limited to, through in vivo delivery of corresponding guide RNAs to the brain of the subject—a symptom of Alzheimer's disease is alleviated, or all symptoms are eliminated.

To target APP, engineered polynucleotides are designed based on a key APP target sequence region listed in SEQ ID NO: 48.

SEQ ID NO: 48
TATCAAGACGGAGGAGATCTCTGAAGTGAAGATGGATGCAGAATTCC
GACATGACTCAGGATATGAAGTTCATCATCAAAAATTGGTGTT

The antisense or complementary sequences (of lengths 20-100 bp) will include sequences complementary to all potential portions of the APP region in SEQ ID NO: 48. The regions opposite the target adenines will be paired with cytosines (as underlined in SEQ ID NO: 48). Examples of antisense sequences are listed in TABLE 9.

TABLE 9
Example antisense sequences for SEQ ID NO: 48.

| SEQ | |
|---|---|
| ID NO | SEQUENCE |
| 49 | TCTGCACCCATCTTCACTTC |
| 50 | TCATATCCTGAGTCATGTCGGAATTCTGCACCCATCTTCAC |
| | TTCAGAGATCTCCTCCGTC |
| 51 | TCATATCCTGAGTCATGCCGGAATTCTGCACCCATCTTCAC |
| | TTCAGAGACCTCCTCCGTC |

The underlined C corresponds to the cytosine paired with the adenine (the underlined A in SEQ ID NO: 48)

The engineered polynucleotide sequences, based on those listed in TABLE 9, are listed in TABLE 10.

TABLE 10
Example engineered polynucleotide sequences
based on sequences in TABLE 9.

| SEQ | |
|---|---|
| ID NO | SEQUENCE |
| 52 | GTGGAATAGTATAACAATATGCTAAATGTTGTTATAGTATC |
| | CCACN_{S} |
| 53 | GTGGAATAGTATAACAATATGCTAAATGTTGTTATAGTATC |
| | CCACN_{S}GTGGAATAGTATAACAATATGCTAAATGTTGTTAT |
| | AGTATCCCAC |
| 54 | GGCCGGGCGCGGTGGCTCACGCCTGTAATCCCAGCACTTTG |
| | GGAGGCCGAGGCGGGGAGATTGCTTGAGCCCAGGAGTTCGA |
| | GACCAGCCTGGGCAACATAGCGAGACCCCGTCTCN_{S}AGCCG |
| | GGCGTGGTGGCGCGCGCCTGTAGTCCCAGCTACTCGGGAGG |
| | CTGAGGCAGGAGGATCGCTTGAGCCCAGGAGTTCGAGGCTG |
| | CAGTGAGCTATGATCGCGCCACTGCACTCCAGCCTGGGCGA |
| | CAGAGCGAGACCCTGTCTC |

Bold N_{S} represent any of the sequences listed in TABLE 9.

### Example 2: Multiplexed Compositions for Treatment of Alzheimer's Disease

This example describes multiplexed compositions (e.g., two engineered guide RNAs of the present disclosure targeting different RNA polynucleotides) for treatment of Alzheimer's disease. A subject is diagnosed with Alzheimer's disease. The subject is prescribed a dosing regimen of a pharmaceutical composition. The pharmaceutical composition comprises a multiplexed composition comprising an engineered guide RNA for editing of a BACE cleavage site (β- or the β′-cleavage site) in APP and an engineered guide RNA targeting a non-protein coding region (e.g., a start site) or causing exon skipping of a start site of the Tau gene, resulting in Tau knockdown in either cases. The pharmaceutical composition is administered to the subject by parenchymal injection, direct injection to cranial tissue, ICM injection, or ICV injection in an effective amount to treat Parkinson's disease. Upon administration of the multiplexed composition to the subject, a symptom of Alzheimer's disease is alleviated, or all symptoms are eliminated.

### Example 3: Combination Treatment of Alzheimer's Disease

This example describes multiplexed compositions for treatment of Alzheimer's disease (e.g., two engineered guide RNAs of the present disclosure targeting different RNA polynucleotides). A subject will be diagnosed with Alzheimer's disease. The subject will be prescribed a dosing regimen of a pharmaceutical composition. The pharmaceutical composition will comprise the multiplexed composition comprising an engineered guide RNA for editing of a BACE cleavage site (β- or the β′-cleavage site) in APP and an engineered guide RNA targeting a non-protein coding region (e.g., a start site or promoter region) of SNCA resulting in alpha-synuclein knockdown. The engineered guide RNA and the antisense oligonucleotide are formulated in a single pharmaceutical composition or separate pharmaceutical compositions. The pharmaceutical composition or pharmaceutical compositions will be administered to the subject by parenchymal injection, direct injection to cranial tissue, ICM injection, or ICV injection in an effective amount to treat Alzheimer's disease. Upon administration of the multiplexed composition to the subject, a symptom of Alzheimer's disease is alleviated, or all symptoms are eliminated.

### Example 4: Multiplexed Vectors for Treatment of Alzheimer's Disease

This example describes multiplexed vectors for treatment of Alzheimer's disease. A subject is diagnosed with Alzheimer's disease. The subject is prescribed a dosing regimen of a pharmaceutical composition. The pharmaceutical composition comprises a first vector comprising an engineered guide RNA targeting APP, and a second vector comprising a second engineered guide RNA targeting Tau-p. The pharmaceutical composition is administered to the subject by direct injection to the central nervous system (CNS), parenchymal injection, direct injection to cranial tissue, ICM injection, or ICV injection in an effective amount to treat Alzheimer's disease. Upon administration of the multiplexed vectors to the subject, a symptom of Alzheimer's disease is alleviated, or all symptoms are eliminated.

### Example 5: Delivery of Multiplexed Engineered Polynucleotides with a Single Vector

Because polymorphisms in different genes are associated with increased risk or severity of Alzheimer's disease, simultaneous manipulation of the expression of at least two target RNAs can be a useful treatment. RNA editing, as illustrated in the current invention, is modular: the RNA editing enzyme and the engineered polynucleotide are two different entities. Therefore, RNA editing can be multiplexed to correct multiple distinct targets simultaneously. For example, to treat Alzheimer's disease patients with contributing polymorphisms in APP and Tau or SNCA, two coding sequences are generated. The first coding sequence codes for any of the guide RNAs capable of binding to a target RNA sequence in any APP isoform, such as any one of those listed in TABLE 1 and 13 or any of the guide RNAs in TABLE 10 or 17. These guide RNAs can edit the cleavage sites in APP to inhibit the production of Abeta 40/42. The second coding sequence codes for a guide RNA that targets the start ATG of Tau or SNCA mRNA listed in TABLE 4 and 6 or any guide RNA listed in TABLEs 14-16. It can convert any nucleotide of the start ATG into any other nucleotide. Since the start ATG is removed, the expression of Tau or SNCA should decrease. These two coding sequences are each paired with a Polymerase III promoter and cloned into a single viral vector—such as an adenoviral vector, an adeno-associated viral vector (AAV), a lentiviral vector, or a retroviral vector—to express both coding sequences. The vector is injected into the brain of the patient by intracerebroventricular injection.

A subject is diagnosed with Alzheimer's disease. The subject is prescribed a dosing regimen of a pharmaceutical composition. The pharmaceutical composition comprises a multiplex targeting scheme of a vector comprising a first polynucleotide encoding a first engineered guide RNA that targets Abeta 40/42 and a second polynucleotide encoding a second engineered guide RNA that targets Tau/SNCA. The pharmaceutical composition is administered to the subject by direct injection to the central nervous system (CNS) in an effective amount to treat Alzheimer's disease.

### Example 6: Delivery of Multiplexed Engineered Polynucleotides with Multiple Vectors

The modularity of the RNA editing entity and the RNA targeting polynucleotide allows the multiplexed targeting to be carried out in various ways; for example, to treat Alzheimer's disease patients with contributing polymorphisms in APP and Tau or SNCA, two coding sequences are generated. The first coding sequence codes for any of the guide RNAs capable of binding to a target RNA sequence in any APP isoform, such as any one of those listed in TABLE 1 and 13 or any of the guide RNAs in TABLE 10 or 17. The second coding sequence codes for an engineered polynucleotide that targets the start ATG of any one of the Tau or SNCA mRNA listed in TABLE 4 and 6 or any guide RNA listed in TABLEs 14-16 and can convert any nucleotide of the start ATG into any other nucleotide. Since the start ATG is removed, the expression of Tau or SNCA should decrease. These two coding sequences are each paired with a Polymerase III promoter and each cloned into a single viral vector-such as an adenoviral vector, an adeno-associated viral vector (AAV), a lentiviral vector, or a retroviral vector-to express each coding sequence individually. The vectors are injected into the brain of the patient by intracerebroventricular injection.

A subject is diagnosed with Alzheimer's disease. The subject is prescribed a dosing regimen of a pharmaceutical composition. The pharmaceutical composition will comprise a multiplex targeting scheme of a first vector comprising a first polynucleotide encoding a first engineered guide RNA that targets Abeta 40/42 and a second vector comprising a second polynucleotide encoding a second engineered polynucleotide that targets Tau/SNCA. The pharmaceutical composition is administered to the subject by direct injection to the central nervous system (CNS) in an effective amount to treat Alzheimer's disease.

### Example 7: Delivery of Multiplexed Engineered Polynucleotides with Multiple Vectors

The engineered polynucleotides described in this invention can be maintained and administered without any viral vectors; for example, a first coding sequence coding for any of the guide RNAs capable of binding to a target RNA sequence in any APP isoform, such as any one of those listed in TABLE 1 and 13 or any of the guide RNAs in TABLE 10 or 17; and a second coding sequence coding for an engineered polynucleotide that targets the start ATG of any one of the Tau or SNCA mRNA listed in TABLE 4 and 6 or any guide RNA listed in TABLEs 14-16, each paired with a Polymerase III promoter, are injected into the brain of the patient by intracerebroventricular injection.

A subject is diagnosed with Alzheimer's disease. The subject is prescribed a dosing regimen of a pharmaceutical composition. The pharmaceutical composition comprises a multiplex targeting scheme of a first vector comprising a first polynucleotide encoding a first engineered guide RNA that targets Abeta 40/42 and a second vector comprising a second polynucleotide encoding a second engineered polynucleotide that targets Tau/SNCA. The pharmaceutical composition is administered to the subject by direct injection to the central nervous system (CNS) in an effective amount to treat Alzheimer's disease.

### Example 8: Delivery of Multiplexed Engineered Polynucleotides with Multiple Vectors

Any engineered polynucleotides described in Example 6, 7, and 8 are combined and injected into the brain of the patient by intracerebroventricular injection.

A subject is diagnosed with Alzheimer's disease. The subject is prescribed a dosing regimen of a pharmaceutical composition. The pharmaceutical composition w comprises a multiplex targeting scheme of a first vector comprising a first polynucleotide encoding a first engineered guide RNA that targets Abeta 40/42 and a second vector comprising a second polynucleotide encoding a second engineered polynucleotide that targets Tau/SNCA. The pharmaceutical composition is administered to the subject by direct injection to the central nervous system (CNS) in an effective amount to treat Alzheimer's disease.

### Example 9: Identification of Target RNA Cleavage Sites within an RNA Encoding APP

### Identification of Target RNA Containing Mutations

Target RNA sites were identified by analyzing human APP amino acid sequences and corresponding nucleotide sequences encoding said amino acid sequences at and/or near to the protease cleavage sites of APP. As can be seen in FIG. 5, APP contains numerous cleavage sites which are cleaved by endogenous proteases, such as beta-secretase (e.g., BACE1, cathepsin B or Meprin beta) and alpha secretases (e.g., ADAM10). Mutant APP proteins are selected that are non-amyloidogenic and/or can be made by ADAR-editing of endogenous APP. Levels of beta amyloid plaque forming metabolites, including amyloid beta 40 (Abeta 40) and amyloid beta 42 (Abeta 42), were measured. Mutations near the β-site, β′-site, and α-site as indicated in FIG. 5 were identified as amenable to editing by ADAR and were selected for further analysis.

### Generation of Cell Lines Expressing Exemplary Mutant APP Polypeptide

Cell lines that express the wild type APP polypeptide provided in SEQ ID NO: 2 and the APP polypeptides with the mutations listed in TABLE 2 were generated. In an exemplary attempt, plasmids encoding the APP695 isoform with the desired mutation listed in TABLE 2 were generated. The APP695 isoform was chosen because it is the most highly expressed in neuron cells but other isoforms can be utilized. A mammalian codon-optimized APP695 isoform was synthesized as a single g-block gene fragment. APP695 was then cloned onto a pBI-CMV-mCherry backbone using Gibson assembly, provide in TABLE 11. A summary of the location of the features of the plasmid is provided in TABLE 12.

TABLE 11
pBI-CMV-mCherry backbone with wild type APP:

| SEQ | |
|---|---|
| ID | |
| NO | DNA |
| 55 | CTCGAGTAGTTATTAATAGTAATCAATTACGGGGTCATTAGTTCAT |
| | AGCCCATATATGGAGTTCCGCGTTACATAACTTACGGTAAATGGCC |
| | CGCCTGGCTGACCGCCCAACGACCCCCGCCCATTGACGTCAATAA |
| | TGACGTATGTTCCCATAGTAACGCCAATAGGGACTTTCCATTGACG |
| | TCAATGGGTGGAGTATTTACGGTAAACTGCCCACTTGGCAGTACAT |
| | CAAGTGTATCATATGCCAAGTACGCCCCCTATTGACGTCAATGACG |
| | GTAAATGGCCCGCCTGGCATTATGCCCAGTACATGACCTTATGGGA |
| | CTTTCCTACTTGGCAGTACATCTACGTATTAGTCATCGCTATTACC |
| | ATGGTGATGCGGTTTTGGCAGTACATCAATGGGCGTGGATAGCGG |
| | TTTGACTCACGGGGATTTCCAAGTCTCCACCCCATTGACGTCAATG |
| | GGAGTTTGTTTTGGCACCAAAATCAACGGGACTTTCCAAAATGTCG |
| | TAACAACTCCGCCCCATTGACGCAAATGGGCGGTAGGCGTGTACG |
| | GTGGGAGGTCTATATAAGCAGAGCTGGTTTAGTGAACCGTCAGAT |
| | CCGCTAGGGATCCTCTAGTCAGCTGACGCGTGCTAGCGCGGCCGC |
| | ATCGATAATGCTCCCTGGACTTGCTTTGCTGCTTTTGGCAGCCTGG |
| | ACTGCTCGAGCACTCGAGGTCCCAACGGATGGAAACGCGGGTCTT |
| | TTGGCAGAGCCTCAAATAGCAATGTTTTGCGGAAGACTCAACATGC |
| | ATATGAACGTTCAGAATGGGAAATGGGACTCCGACCCCAGTGGTA |
| | CGAAGACATGTATTGACACAAAGGAGGGAATACTCCAGTACTGCC |
| | AGGAAGTGTACCCGGAGCTTCAGATTACGAATGTGGTAGAGGCTA |
| | ATCAACCCGTAACTATCCAAAATTGGTGTAAGAGAGGCAGGAAGC |
| | AATGCAAGACTCATCCTCATTTCGTAATTCCGTATCGATGTTTGGT |
| | GGGAGAATTTGTCTCTGACGCATTGCTTGTTCCTGACAAGTGTAAG |
| | TTTCTTCACCAGGAACGCATGGACGTGTGCGAGACACACTTGCACT |
| | GGCATACCGTTGCGAAGGAGACGTGTTCCGAAAAGAGTACAAATC |
| | TCCATGACTACGGCATGTTGCTCCCGTGCGGAATAGATAAGTTCCG |
| | AGGCGTGGAGTTTGTATGCTGTCCGCTGGCAGAGGAGAGCGATAA |
| | TGTCGATTCCGCAGATGCCGAAGAGGACGACAGCGACGTCTGGTG |
| | GGGAGGAGCGGACACTGATTACGCTGATGGTAGTGAGGACAAAGT |
| | AGTCGAGGTGGCAGAAGAAGAAGAAGTGGCGGAGGTTGAAGAAG |
| | AAGAGGCAGACGATGACGAAGACGATGAGGACGGTGATGAGGTAG |
| | AAGAAGAAGCGGAAGAACCGTACGAAGAAGCTACGGAACGCACTA |
| | CAAGTATTGCTACCACTACAACCACTACAACCGAATCAGTTGAGGA |
| | AGTGGTGCGAGTCCCCACTACGGCTGCCAGTACACCGGATGCCGT |
| | CGACAAATACCTGGAGACTCCTGGCGACGAAAACGAACATGCTCA |
| | TTTCCAGAAGGCGAAGGAACGCCTCGAAGCAAAGCACAGAGAGAG |
| | AATGTCACAGGTAATGAGGGAATGGGAGGAGGCGGAACGCCAAGC |
| | AAAGAACCTGCCTAAAGCGGACAAGAAGGCAGTTATCCAACATTTC |
| | CAAGAGAAAGTGGAGAGTCTCGAACAGGAGGCAGCGAACGAGAG |
| | GCAACAATTGGTAGAAACGCACATGGCGAGGGTGGAAGCTATGCT |
| | CAATGACCGAAGACGACTTGCCTTGGAAAATTACATTACTGCCCTT |
| | CAAGCCGTCCCACCGCGCCCACGCCATGTCTTTAACATGCTTAAGA |
| | AGTATGTTCGAGCTGAACAGAAGGATCGGCAACACACCCTGAAAC |
| | ACTTCGAACATGTCAGAATGGTTGACCCGAAGAAGGCTGCACAGA |
| | TTCGAAGTCAAGTTATGACCCATTTGAGGGTAATATATGAGAGAAT |
| | GAACCAAAGTCTGAGCCTTCTCTACAATGTCCCCGCTGTGGCCGAG |
| | GAAATTCAGGACGAAGTCGATGAGCTCCTGCAAAAGGAGCAGAAC |
| | TACTCTGACGATGTACTTGCTAATATGATTTCAGAGCCAAGGATCA |
| | GTTATGGAAACGACGCCCTGATGCCTAGTCTTACCGAAACCAAGAC |
| | TACGGTAGAACTCCTTCCCGTTAACGGAGAGTTCAGCTTGGACGAC |
| | CTTCAGCCTTGGCACTCATTCGGAGCTGATTCCGTACCAGCCAATA |
| | CGGAGAATGAAGTAGAGCCCGTAGACGCAAGACCTGCAGCGGACA |
| | GAGGGCTGACGACGAGACCCGGTAGCGGTTTGACAAATATCAAGA |
| | CGGAGGAGATCTCTGAAGTGAAGATGGATGCAGAATTCCGACATG |
| | ACTCAGGATATGAAGTTCATCATCAAAAATTGGTGTTCTTTGCAGA |
| | AGATGTCGGTTCTAACAAGGGTGCTATCATAGGCCTTATGGTGGGT |
| | GGCGTCGTGATTGCGACCGTGATAGTTATTACGCTTGTCATGCTGA |
| | AGAAGAAACAGTATACGTCCATCCATCACGGTGTGGTAGAGGTAG |
| | ATGCGGCCGTAACTCCCGAAGAGCGCCATCTTTCTAAGATGCAGC |
| | AGAATGGATACGAGAACCCCACGTACAAATTCTTTGAGCAAATGCA |
| | AAACTGATGTCGACGATATCTCCAGAGGATCATAATCAGCCATACC |
| | ACATTTGTAGAGGTTTTACTTGCTTTAAAAAACCTCCCACACCTCC |
| | CCCTGAACCTGAAACATAAAATGAATGCAATTGTTGTTGTTAACTT |
| | GTTTATTGCAGCTTATAATGGTTACAAATAAAGCAATAGCATCACA |
| | AATTTCACAAATAAAGCATTTTTTTCACTGCCCCGAGCTTCCTCGC |
| | TCACTGACTCGCTGCGCTCGGTCGTTCGGCTGCGGCGAGCGGTAT |
| | CAGCTCACTCAAAGGCGGTAATACGGTTATCCACAGAATCAGGGG |
| | ATAACGCAGGAAAGAACATGTGAGCAAAAGGCCAGCAAAAGGCCA |
| | GGAACCGTAAAAAGGCCGCGTTGCTGGCGTTTTTCCATAGGCTCC |
| | GCCCCCCTGACGAGCATCACAAAAATCGACGCTCAAGTCAGAGGT |
| | GGCGAAACCCGACAGGACTATAAAGATACCAGGCGTTTCCCCCTG |
| | GAAGCTCCCTCGTGCGCTCTCCTGTTCCGACCCTGCCGCTTACCGG |
| | ATACCTGTCCGCCTTTCTCCCTTCGGGAAGCGTGGCGCTTTCTCAT |
| | AGCTCACGCTGTAGGTATCTCAGTTCGGTGTAGGTCGTTCGCTCCA |
| | AGCTGGGCTGTGTGCACGAACCCCCCGTTCAGCCCGACCGCTGCG |
| | CCTTATCCGGTAACTATCGTCTTGAGTCCAACCCGGTAAGACACGA |
| | CTTATCGCCACTGGCAGCAGCCACTGGTAACAGGATTAGCAGAGC |
| | GAGGTATGTAGGCGGTGCTACAGAGTTCTTGAAGTGGTGGCCTAA |
| | CTACGGCTACACTAGAAGAACAGTATTTGGTATCTGCGCTCTGCTG |
| | AAGCCAGTTACCTTCGGAAAAAGAGTTGGTAGCTCTTGATCCGGCA |
| | AACAAACCACCGCTGGTAGCGGTGGTTTTTTTGTTTGCAAGCAGCA |
| | GATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTTT |
| | TAAATTAAAAATGAAGTTTTAAATCAATCTAAAGTATATATGAGTA |
| | AACTTGGTCTGACAGTTACCAATGCTTAATCAGTGAGGCACCTATC |
| | TCAGCGATCTGTCTATTTCGTTCATCCATAGTTGCCTGACTCCCCG |
| | TCGTGTAGATAACTACGATACGGGAGGGCTTACCATCTGGCCCCA |
| | GTGCTGCAATGATACCGCGAGACCCACGCTCACCGGCTCCAGATT |
| | TATCAGCAATAAACCAGCCAGCCGGAAGGGCCGAGCGCAGAAGTG |
| | GTCCTGCAACTTTATCCGCCTCCATCCAGTCTATTAATTGTTGCCG |
| | GGAAGCTAGAGTAAGTAGTTCGCCAGTTAATAGTTTGCGCAACGTT |
| | GTTGCCATTGCTACAGGCATCGTGGTGTCACGCTCGTCGTTTGGTA |
| | TGGCTTCATTCAGCTCCGGTTCCCAACGATCAAGGCGAGTTACATG |
| | ATCCCCCATGTTGTGCAAAAAAGCGGTTAGCTCCTTCGGTCCTCCG |
| | ATCGTTGTCAGAAGTAAGTTGGCCGCAGTGTTATCACTCATGGTTA |
| | TGGCAGCACTGCATAATTCTCTTACTGTCATGCCATCCGTAAGATG |
| | CTTTTCTGTGACTGGTGAGTACTCAACCAAGTCATTCTGAGAATAG |
| | TGTATGCGGCGACCGAGTTGCTCTTGCCCGGCGTCAACACGGGAT |
| | AATACCGCGCCACATAGCAGAACTTTAAAAGTGCTCATCATTGGAA |
| | AACGTTCTTCGGGGCGAAAACTCTCAAGGATCTTACCGCTGTTGAG |
| | ATCCAGTTCGATGTAACCCACTCGTGCACCCAACTGATCTTCAGCA |
| | TCTTTTACTTTCACCAGCGTTTCTGGGTGAGCAAAAACAGGAAGGC |
| | AAAATGCCGCAAAAAAGGGAATAAGGGCGACACGGAAATGTTGAAT |
| | ACTCATACTCTTCCTTTTTCAATATTATTGAAGCATTTATCAGGGT |
| | TATTGTCTCATGAGCGGATACATATTTGAATGTATTTAGAAAAATA |
| | AACAAATAGGGGTTCCGCGCACATTTCCCCGAAAAGTGCCACCTG |
| | ACGTCGGCAGTGAAAAAAATGCTTTATTTGTGAAATTTGTGATGCT |
| | ATTGCTTTATTTGTAACCATTATAAGCTGCAATAAACAAGTTAACA |
| | ACAACAATTGCATTCATTTTATGTTTCAGGTTCAGGGGGAGGTGTG |
| | GGAGGTTTTTTAAAGCAAGTAAAACCTCTACAAATGTGGTATGGCT |
| | GATTATGATCCTCTAGACTGCAGCCTCAGGAGATCTGGGCCCCCG |
| | CGGCATATGACCGGTGCTACTTGTACAGCTCGTCCATGCCGCCGG |
| | TGGAGTGTCTACCCTCGGCGCGTTCGTACTGTTCCACGATGGTGTA |
| | GTCCTCGTTGTGGGAGGTGATGTCCAACTTGATGTTGACGTTGTAG |
| | GCGCCGGGCAGCTGCACGGGCTTCTTGGCCTTGTAGGTGGTCTTG |
| | ACCTCAGCGTCGTAGTGGCCGCCGTCCTTCAGCTTCAGCCTCTGCT |
| | TGATCTCGCCCTTCAGGGCGCCGTCCTCGGGGTACATCCGCTCGG |
| | AGGAGGCCTCCCAGCCCATGGTCTTCTTCTGCATTACGGGGCCGT |
| | CGGAGGGGAAGTTGGTGCCGCGCAGCTTCACCTTGTAGATGAACT |
| | CGCCGTCTTGCAGGGAGGAGTCCTGGGTCACGGTCACCACGCCGC |
| | CGTCCTCGAAGTTCATCACGCGCTCCCACTTGAAGCCCTCGGGGA |
| | AGGACAGCTTCAAGTAGTCGGGGATGTCGGCGGGGTGCTTCACGT |
| | AGGCCTTGGAGCCGTACATGAATTGAGGGGACAGGATGTCCCAGG |
| | CGAAGGGCAGGGGGCCACCCTTGGTCACCTTCAGCTTGGCGGTCT |
| | GGGTGCCCTCGTAGGGGCGACCTTCACCCTCGCCCTCGATCTCGA |
| | ACTCGTGGCCGTTCACGGAGCCCTCCATGTGCACCTTGAAGCGCA |
| | TGAACTCCTTGATGATGGCCATGTTATCCTCCTCGCCCTTAGAAAC |
| | CATCTCCAGGCGATCTGACGGTTCACTAAACGAGCTCTGCTTATAT |
| | AGGCCTCCCACCGTACACGCCACCTCGACATA |

TABLE 12
Features of the pBI-CMV-mCherry backbone
with wild type APP of SEQ ID NO 48

| | Location of the |
|---|---|
| Feature | feature (bp) |
| Enhancer | 64 to 473 |
| Minimal CMV Promoter #1 | 474 to 599 |
| MCS #1 | 601 to 646 |
| CDS1 (WT APP695 isoform) | 647 to 2734 |
| NM_201414.3 | 2393 to 2500 |
| BACE Cleavage Site | 2432 to 2437 |
| ADAM10 Cleavage Site | 2480 to 2482 |
| MCS #2 | 2735 to 2747 |
| SV40 polyA #1 | 2759 to 2946 |
| pUC origin | 3122 to 3721 |
| AmpR | 3883 to 4743 |
| SV40 polyA #2 | 4879 to 5066 |
| MCS #3 | 5070 to 5119 |
| CDS2 (mCherry) | 5120 to 5119 |
| MCS #4 | 5831 to 5834 |
| Minimal CMV Promoter #2 | 5837 to 5905 |

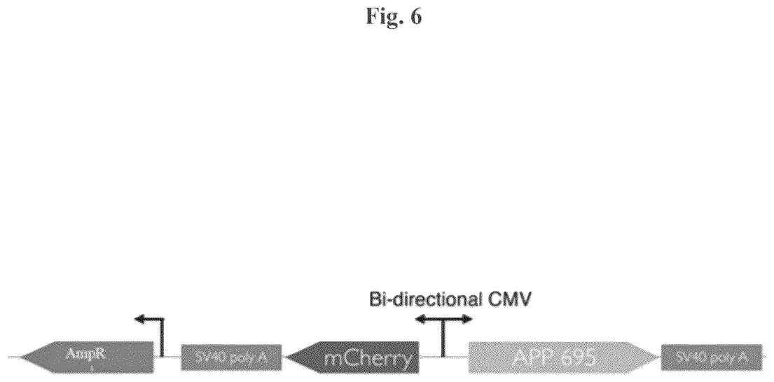

Exemplary portions of plasmid sequences that each comprise a mutation that generates an APP polypeptide with the mutations listed in TABLE 2 are shown in FIG. 6. All plasmids containing these mutant APP gene sequences in the CDS1 region (bases 647 to 2734) of SEQ ID NO: 56-70, instead of the WT APP695 isoform are listed in the CDS1 of TABLE 12 in SEQ ID NO: 55. These sequences are listed in TABLE 13.

TABLE 13
Human Mutant APP mRNA Isoform Sequences

| SEQ | | |
|---|---|---|
| ID NO | Mutation | mRNA Sequence |
| 56 | K670R | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAG |
| | | GAUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 57 | K670E | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGGA |
| | | GAUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 58 | K670G | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGGG |
| | | GAUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 59 | K670R + | AUGCUCCCUGGACUUGCUUUGCUGCUUUUGGCAG |
| | M671V | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAG |
| | | GGUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 60 | K670E + | AUGCUCCCUGGACUUGCUUUGCUGCUUUUGGCAG |
| | M671V | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGGA |
| | | GGUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 61 | K670G + | AUGCUCCCUGGACUUGCUUUGCUGCUUUUGGCAG |
| | M671V | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGGG |
| | | GGUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 62 | M671V | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GGUGGAUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 63 | D672G | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGGUGCAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 64 | A673V | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGUGGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 65 | A673T | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUACAGAAUUCCGACAUGACUCAGGAUAU |
| | | GAAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 66 | K687R | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGCAGAAUUCCGACAUGACUCAGGAUAU |
| | | GAAGUUCAUCAUCAAAGAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 67 | K687E | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGCAGAAUUCCGACAUGACUCAGGAUAU |
| | | GAAGUUCAUCAUCAAGAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 68 | K687G | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGCAGAAUUCCGACAUGACUCAGGAUAU |
| | | GAAGUUCAUCAUCAAGGAUUGGUGUUCUUUGCAG |
| | | AAGAUGUCGGUUCUAACAAGGGUGCUAUCAUAGG |
| | | CCUUAUGGUGGGUGGCGUCGUGAUUGCGACCGUG |
| | | AUAGUUAUUACGCUUGUCAUGCUGAAGAAGAAACA |
| | | GUAUACGUCCAUCCAUCACGGUGUGGUAGAGGUA |
| | | GAUGCGGCCGUAACUCCCGAAGAGCGCCAUCUUU |
| | | CUAAGAUGCAGCAGAAUGGAUACGAGAACCCCAC |
| | | GUACAAAUUCUUUGAGCAAAUGCAAAACUGA |
| 69 | H684R | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGCAGAAUUCCGACAUGACUCAGGAUAU |
| | | GAAGUUCGUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |
| 70 | E682G | AUGCUCCCUGGACUUGCUUUGCUGCUUUUGGCAG |
| | | CCUGGACUGCUCGAGCACUCGAGGUCCCAACGGA |
| | | UGGAAACGCGGGUCUUUUGGCAGAGCCUCAAAUA |
| | | GCAAUGUUUUGCGGAAGACUCAACAUGCAUAUGAA |
| | | CGUUCAGAAUGGGAAAUGGGACUCCGACCCCAGU |
| | | GGUACGAAGACAUGUAUUGACACAAAGGAGGGAA |
| | | UACUCCAGUACUGCCAGGAAGUGUACCCGGAGCU |
| | | UCAGAUUACGAAUGUGGUAGAGGCUAAUCAACCC |
| | | GUAACUAUCCAAAAUUGGUGUAAGAGAGGCAGGA |
| | | AGCAAUGCAAGACUCAUCCUCAUUUCGUAAUUCCG |
| | | UAUCGAUGUUUGGUGGGAGAAUUUGUCUCUGACG |
| | | CAUUGCUUGUUCCUGACAAGUGUAAGUUUCUUCAC |
| | | CAGGAACGCAUGGACGUGUGCGAGACACACUUGC |
| | | ACUGGCAUACCGUUGCGAAGGAGACGUGUUCCGA |
| | | AAAGAGUACAAAUCUCCAUGACUACGGCAUGUUGC |
| | | UCCCGUGCGGAAUAGAUAAGUUCCGAGGCGUGGA |
| | | GUUUGUAUGCUGUCCGCUGGCAGAGGAGAGCGAU |
| | | AAUGUCGAUUCCGCAGAUGCCGAAGAGGACGACA |
| | | GCGACGUCUGGUGGGGAGGAGCGGACACUGAUUA |
| | | CGCUGAUGGUAGUGAGGACAAAGUAGUCGAGGUG |
| | | GCAGAAGAAGAAGAAGUGGCGGAGGUUGAAGAAG |
| | | AAGAGGCAGACGAUGACGAAGACGAUGAGGACGG |
| | | UGAUGAGGUAGAAGAAGAAGCGGAAGAACCGUAC |
| | | GAAGAAGCUACGGAACGCACUACAAGUAUUGCUAC |
| | | CACUACAACCACUACAACCGAAUCAGUUGAGGAAG |
| | | UGGUGCGAGUCCCCACUACGGCUGCCAGUACACC |
| | | GGAUGCCGUCGACAAAUACCUGGAGACUCCUGGC |
| | | GACGAAAACGAACAUGCUCAUUUCCAGAAGGCGAA |
| | | GGAACGCCUCGAAGCAAAGCACAGAGAGAGAAUG |
| | | UCACAGGUAAUGAGGGAAUGGGAGGAGGCGGAAC |
| | | GCCAAGCAAAGAACCUGCCUAAAGCGGACAAGAA |
| | | GGCAGUUAUCCAACAUUUCCAAGAGAAAGUGGAG |
| | | AGUCUCGAACAGGAGGCAGCGAACGAGAGGCAAC |
| | | AAUUGGUAGAAACGCACAUGGCGAGGGUGGAAGC |
| | | UAUGCUCAAUGACCGAAGACGACUUGCCUUGGAAA |
| | | AUUACAUUACUGCCCUUCAAGCCGUCCCACCGCGC |
| | | CCACGCCAUGUCUUUAACAUGCUUAAGAAGUAUGU |
| | | UCGAGCUGAACAGAAGGAUCGGCAACACACCCUG |
| | | AAACACUUCGAACAUGUCAGAAUGGUUGACCCGAA |
| | | GAAGGCUGCACAGAUUCGAAGUCAAGUUAUGACC |
| | | CAUUUGAGGGUAAUAUAUGAGAGAAUGAACCAAA |
| | | GUCUGAGCCUUCUCUACAAUGUCCCCGCUGUGGCC |
| | | GAGGAAAUUCAGGACGAAGUCGAUGAGCUCCUGC |
| | | AAAAGGAGCAGAACUACUCUGACGAUGUACUUGCU |
| | | AAUAUGAUUUCAGAGCCAAGGAUCAGUUAUGGAAA |
| | | CGACGCCCUGAUGCCUAGUCUUACCGAAACCAAGA |
| | | CUACGGUAGAACUCCUUCCCGUUAACGGAGAGUUC |
| | | AGCUUGGACGACCUUCAGCCUUGGCACUCAUUCG |
| | | GAGCUGAUUCCGUACCAGCCAAUACGGAGAAUGA |
| | | AGUAGAGCCCGUAGACGCAAGACCUGCAGCGGAC |
| | | AGAGGGCUGACGACGAGACCCGGUAGCGGUUUGA |
| | | CAAAUAUCAAGACGGAGGAGAUCUCUGAAGUGAA |
| | | GAUGGAUGCAGAAUUCCGACAUGACUCAGGAUAU |
| | | GGAGUUCAUCAUCAAAAAUUGGUGUUCUUUGCAGA |
| | | AGAUGUCGGUUCUAACAAGGGUGCUAUCAUAGGC |
| | | CUUAUGGUGGGUGGCGUCGUGAUUGCGACCGUGA |
| | | UAGUUAUUACGCUUGUCAUGCUGAAGAAGAAACAG |
| | | UAUACGUCCAUCCAUCACGGUGUGGUAGAGGUAG |
| | | AUGCGGCCGUAACUCCCGAAGAGCGCCAUCUUUCU |
| | | AAGAUGCAGCAGAAUGGAUACGAGAACCCCACGU |
| | | ACAAAUUCUUUGAGCAAAUGCAAAACUGA |

The APP695 plasmid was subjected to site-directed mutagenesis in order to make the desired APP mutants listed in TABLE 13. Nucleotide substitutions for each target mutant at or near the BACE cleavage site (β-site and β′-site) are shown in FIG. 7, along with nucleotide substitutions corresponding to the A673V mutation (pathogenic control) and A673T mutation (protective mutation control). Boxes in the nucleotide sequence indicate the target mutation. Nucleotide substitutions for each target mutant at or near the ADAM10 cleavage site (α-site) are shown in FIG. 8. Once prepared, plasmids comprising the desired target mutations were purified using a commercially available plasmid Midiprep kit.

In total, 15 APP constructs specified in TABLE 13 as well as wild type APP in the CDS1 of TABLE 12 in SEQ ID NO: 55 were prepared, and the identity of each construct was confirmed by sequencing.] Sequencing can comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

### In Vitro APP Cleavage Assay

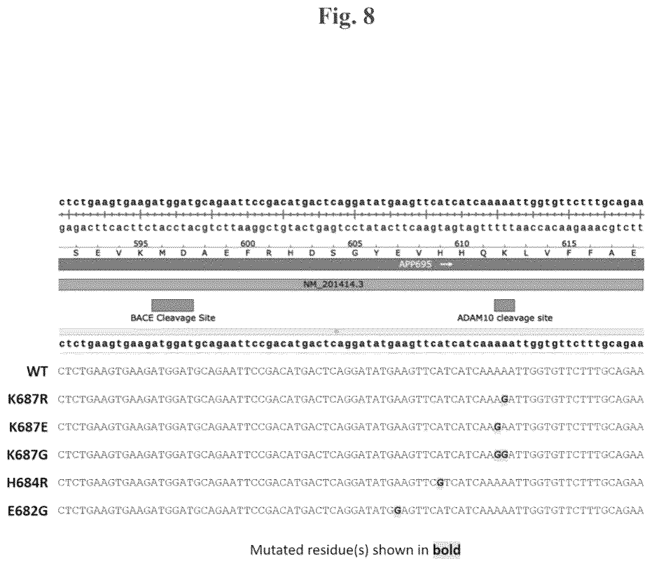

To evaluate the susceptibility of each APP mutant polypeptide to cleavage by endogenous proteases and production of Abeta 40 and Abeta 42 metabolites, a HEK293 APP knockout clone cell line was generated. In brief, HEK293 cells were nucleofected with a combination of 3 CRISPR/Cas9 ribonucleoproteins (RNPs) targeting the intron between exons 5 and 6 of the APP polypeptides. The cells were then cloned by limiting dilution and screened by Western blot to identify cells in which APP was knocked out. A resulting Western blot for isolated cell lines with successful APP knock out is shown FIG. 9. Western blots were visualized following treatment with secondary antibody (m-IgGk BP-HRP, Santa Cruz sc-516102, 1:1000 dilution).

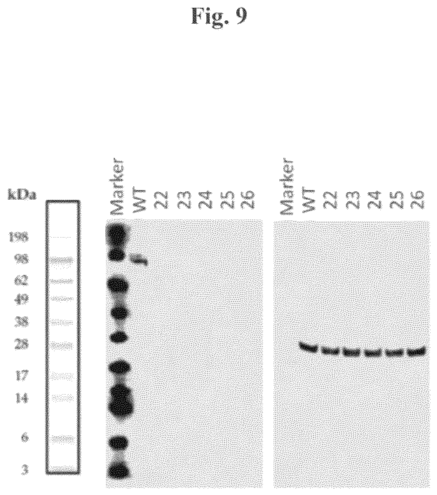

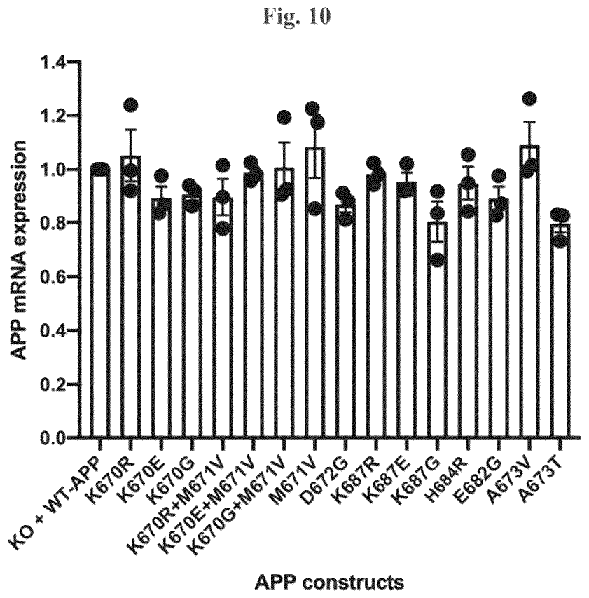

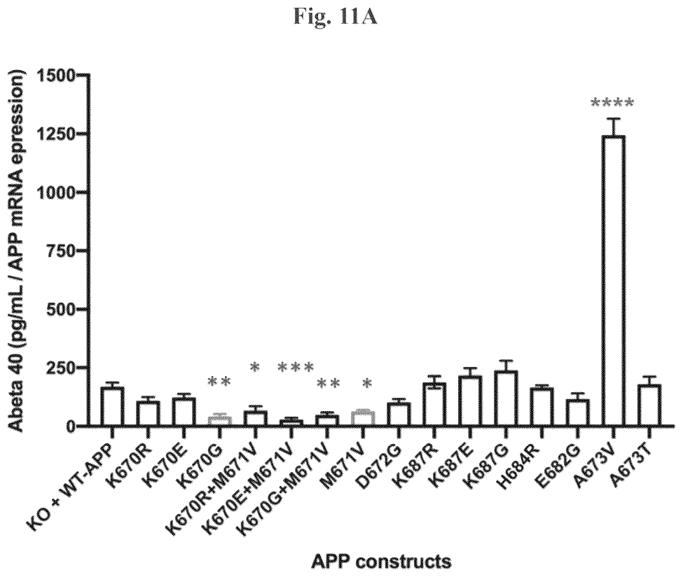

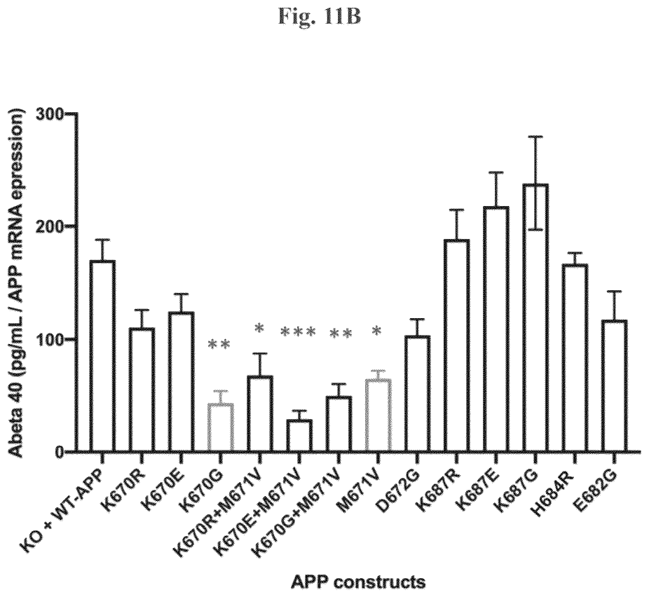

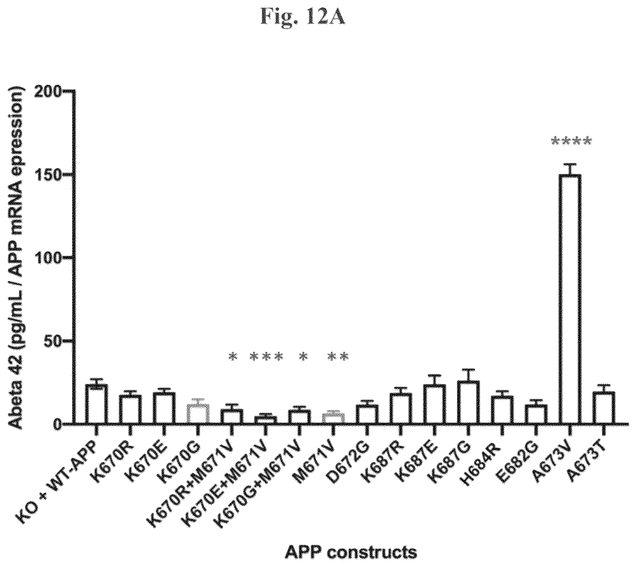

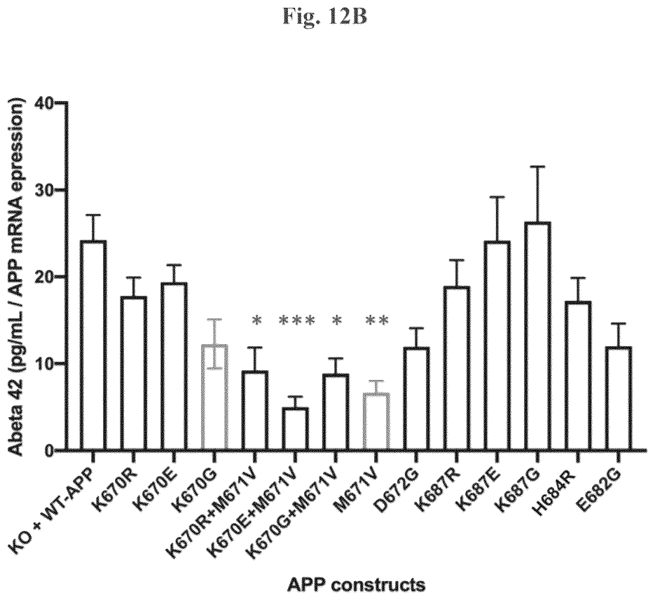

The HEK293 APP knockout cell line was then transfected with 300 ng of the APP constructs (including wild type and controls) at 50,000 cells/well using Transit-293 in a 96-well format. Cells were then cultured for 48 hours post transfection, at which point media and cell lysates were collected. Secreted Abeta 40 and Abeta 42 levels were measured using Quantikine ELISAs (R&D Systems DAB140B and DAB142 kits). The experiment was performed with three separate transfections on three separate days (three screens) for a total of nine samples for each construct. Transfection efficiency was assessed by APP mRNA expression relative to HPRT mRNA levels in the same well using primers respectively conjugated to FAM and VIC. APP expression levels relative to HPRT levels measured this way from a representative well in each of the 3 screens is shown in FIG. 10. No significant difference in APP mRNA expression among constructs was observed (One-way ANOVA). FIG. 11A shows Abeta 40 levels normalized to APP mRNA expression detected upon each day of transfection for each of the tested constructs, including the wild-type APP. Each bar shows the mean+/−SEM. The construct containing the A673V pathogenic mutation showed greatly increased Abeta 40 levels compared to all other groups, as predicted. A One-way ANOVA (P<0.0001) comparing each sample to the knockout plus wild-type APP (KO+WT-APP) demonstrated significant differences. Stars represent statistical significance between the respective APP construct and the KO+WT-APP construct using Dunnett's post-hoc test. K670G and M671V appear to give the most statistically significant reduction in Abeta40 levels normalized to APP mRNA expression. FIG. 12A shows Abeta42 levels normalized to APP mRNA expression detected upon each day of transfection for each of the tested constructs, including the wild-type APP. Each bar shows the mean+/−SEM. The construct containing the A673V pathogenic mutation showed greatly increased Abeta 42 levels compared to all other groups, as predicted. A One-way ANOVA (P<0.0001) comparing each sample to the knockout plus wild-type APP (KO+WT-APP) demonstrated significant differences. Stars represent statistical significance between the respective APP construct and the KO+WT-APP construct using Dunnett's post-hoc test. The M671V mutation appears to give the most statistically significant reduction in Abeta42 levels normalized to APP mRNA expression.

### Example 10: Engineered Polynucleotide Editing of Target RNA

Engineered polynucleotides, for example guide RNA, that comprise targeting sequence to Abeta 40 and/or Abeta 42 are used to correct APP mutation-comprising mRNA. EBV transformed B cells heterozygous for the mutation are treated with the polynucleotide.

In brief, guides are nucleofected in LCL cells using the Lonza X nucleofector, with program EH100. ˜ 40 nmol or 60 nmol of each IVT guide RNA are nucleofected either into ˜2×10⁵ LCL cells per reaction condition. The reaction is split into 2 wells, containing 1×10⁵ each so that the cells can be collected for RNA isolation at either 3 hrs or 7 hrs. At collection, cells are spun at 1,500×g for 1 min. The media is removed. 180 ul of RLT buffer+BMe is added to each well. Qiagen RNeasy protocol and kit are used to isolate the RNAs from the cells. New England Biolabs (NEB) ProtoScript II First-Strand cDNA synthesis kit is used to synthesize cDNA from the isolated RNA. cDNA of APP was sequenced by Sanger sequencing. Sequencing was outsourced to Genewhiz. Sanger traces are analyzed to assess the editing efficiency of each IVT guide.

### Example 11: Beta secretase Cleavage of NRG1

This example describes beta secretase (e.g., BACE1, Cathepsin B, or Meprin beta) cleavage of another beta secretase substrate, Neuregulin1 (NRG1). A HEK293 APP knockout cell line is transfected with 300 ng of mutant APP constructs and a wild type APP construct at 50,000 cells/well using Transit-293 in a 96-well format. Cells are then cultured for 48 hours post transfection. Media and cell lysate are collected. Optionally, a beta secretase inhibitor is added to media and cell lysate immediately prior to sample processing. Secreted Abeta 40 and Abeta 42 levels are measured using Quantikine ELISAs (R&D Systems DAB140B and DAB142 kits) using a portion of the media and/or the lysate. A portion of the media and/or cell lysate is analyzed by liquid chromatography/mass spectrometry to quantify the amount of a peptide metabolite generated upon cleavage of neuregulin 1 (NRG1) by a beta secretase. For example, the cleavage product of NRG1 by a beta secretase between amino acid F237 and M238 is measured and quantified. The amount of peptide quantified is optionally normalized to the amount of full-length NRG1 present in the cell 48 hours post transfection. The amount of full-length NRG1 and the metabolites generated from beta secretase-mediated cleavage of NRG1 present in cells transfected with the APP constructs is measured by an NRG1 ELISA or other method suitable for quantifying the amount of NRG1 present. The amount of NRG1 cleavage product or the ratio of NRG1 cleavage product to the amount of NRG1 present in the cell transfected with the APP mutant is then compared to the amount of NRG1 cleavage product or the ratio of NRG1 cleavage product to the amount of NRG1 present in the cell transfected with the wild type APP construct. A desired APP mutant transfected in a cell has substantially the same profile of NRG1 cleavage product compared to the wild type APP transfected cell.

The experiment described in this example can be modified to measure cleavage products of other proteins targeted by beta secretase which produce metabolites indicative of beta secretase cleavage. Non-limiting examples of other such proteins include amyloid-like protein 1 (APLP1), amyloid-like protein 2 (APLP2), Contactin 2, Jagged 1, neural cell adhesion molecule L1 (CHL1), Neurexin 1α, Neurexin 3β, seizure related protein 6 (SEZ6), seizure related protein 6 precursor protein (SEZ6L), a β (B1-4) Auxiliary subunit of the voltage-gated sodium ion channel (VGSC) subtype Nav1, and VGSC Accessory Subunits KCNE1 or KCNE2.

While preferred embodiments have been shown and described herein, it will be obvious to those skilled in the art that such embodiments are provided by way of example only. Numerous variations, changes, and substitutions will now occur to those skilled in the art. It should be understood that various alternatives to the embodiments described herein may be employed. It is intended that the following claims define the scope and that methods and structures within the scope of these claims and their equivalents be covered herein.

### Example 12: Editing RAB7a and SNCA mRNA

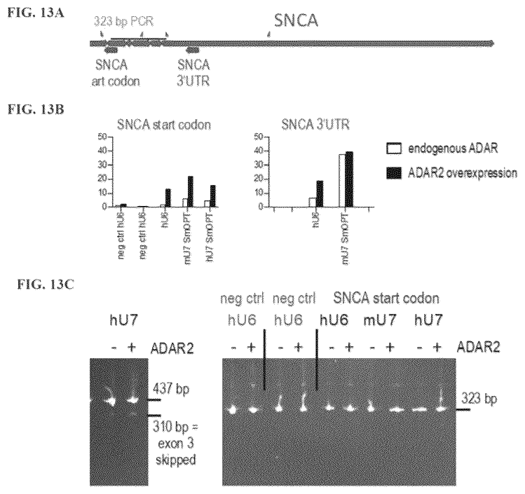

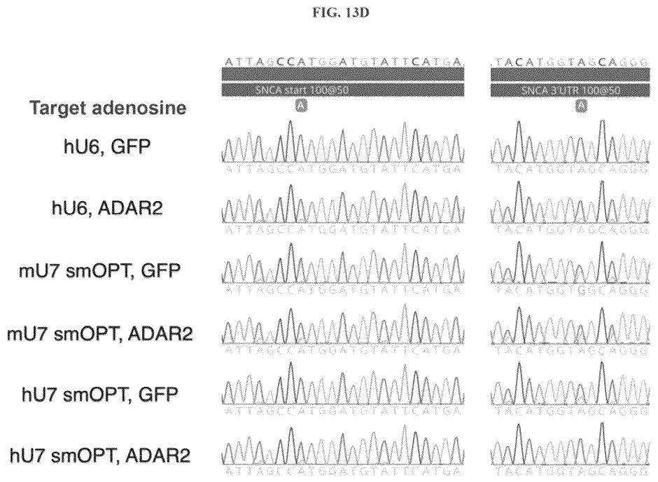

In this example, different regions RAB7a and alpha-synuclein (SNCA) were edited using different guide RNA constructs. For RAB7a, exons 1, 2, and the 3′ UTR were targeted for editing, whereas the start codon and the 3′ UTR of SNCA were targeted. FIG. 13A shows a schematic of the exon structure of human RAB7A and SNCA. Exons are shown as gray segments; the coding region is denoted as a black line above. Locations of the guide RNA targeting sites are shown in purple; PCR primers are shown in green.

100 nt guide RNAs targeting human RAB7A exon 1, exon 3, or 3′UTR, or human SNCA start codon or 3′ UTR were expressed using the hU6 promoter without a 3′ hairpin or the mU7 or hU7 promoters with a 3′ SmOPT U7 hairpin. FIG. 13B summarizes the results of the editing using the different guide RNA constructs in the presence or absence of ADAR2 overexpression. U7 promoters combined with a 3′ SmOPT U7 hairpin enhanced ADAR editing at each target site (measured by Sanger sequencing). While constructs targeting the 3′UTRs worked equally well under endogenous versus overexpressed ADAR levels, constructs targeting other areas still benefited from ADAR2 overexpression.

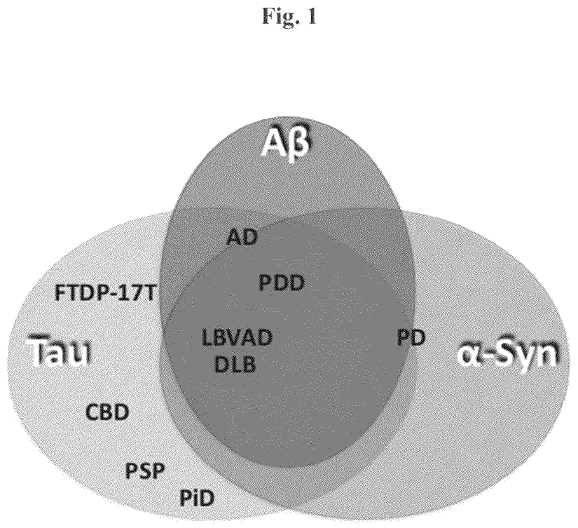

To confirm whether differential exon selection occurred, cDNA derived from the edited transcripts were isolated and PCR amplified using the denoted primers. The RAB7a primers, which span the coding determining sequence of RAB7a, generate a 437 bp amplicon if the exon structure is maintained. If exon 3 of RAB7a is skipped, a 310 bp amplicon is expected. Using the SNCA primers, a 323 bp PCR amplicon is expected. FIG. 13C shows minimal exon 3 skipping. FIG. 13D shows Sanger sequencing chromatograms show specific editing at the target adenosine of the indicated transcripts. The box indicates the on-target editing site. In some embodiments, disclosed herein are compositions of engineered guide RNAs under a U7 promoter and also comprising a smOpt hairpin sequence. Said engineered guide RNAs can hybridize to a target RNA sequence corresponding to SNCA, to facilitate ADAR-mediated editing of an adenosine (see FIG. 1). Editing of the SNCA gene was assessed by transfection in K562 cells highly overexpressing SNCA. At left is a Sanger sequence trace showing on target editing (as denoted by “Target A”) of 91%.

### Example 13: On and off target editing of the 3′ UTR of SNCA in K562 Cells

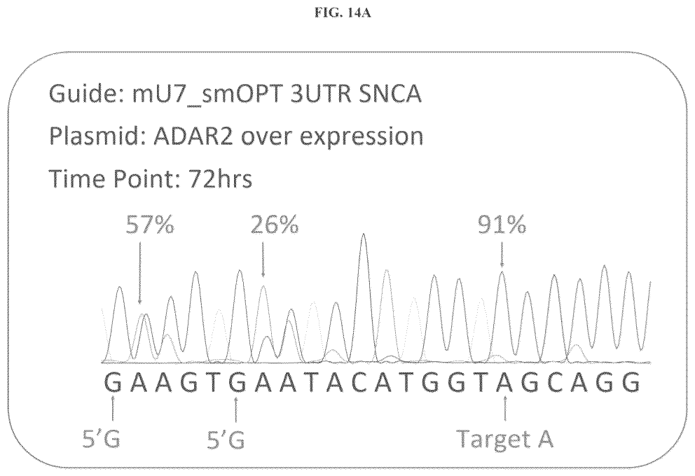

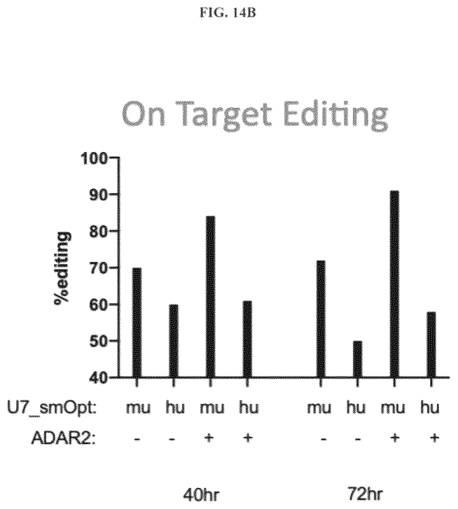

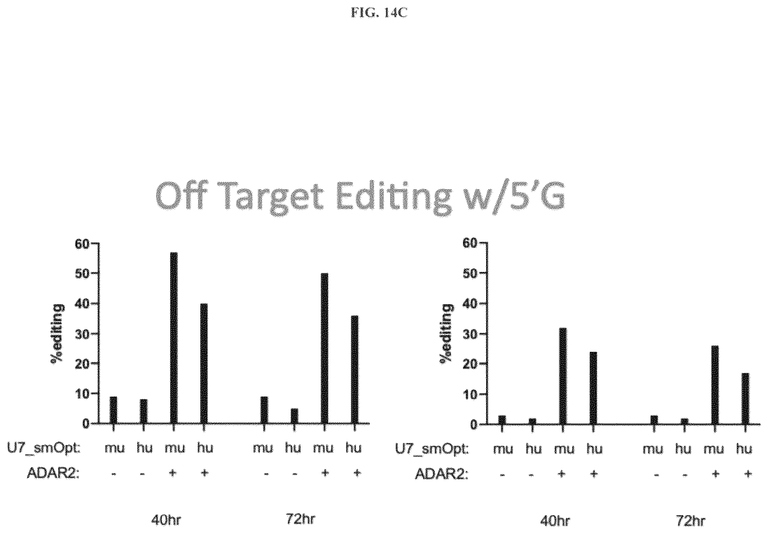

Disclosed herein are compositions of engineered guide RNAs under a U7 promoter and also comprising a smOpt hairpin sequence. Said engineered guide RNAs can hybridize to a target RNA sequence corresponding to SNCA, to facilitate ADAR-mediated editing of an adenosine (see FIG. 14). Editing of the SNCA gene was assessed by transfection of the engineered guide RNAS in K562 cells which overexpress SNCA. 1.5 μg of the engineered guide RNA was transfected into 2×10\hat{ }5 SNCA-overexpressing K562 cells via nucleofection (Lonza). RNA editing was measured 40 and 72 hours after transfection. FIG. 14A is a Sanger sequence chromatogram showing on target editing (as denoted by “Target A”) of 91%. FIG. 14B is a graph showing on target editing at the 40 hour and 72 hour timepoints in K562 cells with and without ADAR2 under either a mouse U7 promoter or a human U7 promoter. High levels of editing (greater than 40% for all constructs) over a sustained period of time were observed. FIG. 14C depicts graphs showing off-target editing of adenosines having a G directly

### Example 14: Regulating the protein expression of SNCA with RNA editing

Disclosed Herein are Methods for Regulating the SNCA Protein Expression Through RNA Editing

HEK293 cells were transfected with plasmids containing the target engineered polynucleotides (Guide A and Guide B) and shRNAs (shRNA1 and shRNA2) against the SNCA mRNA and mock gRNA (as a negative control). Guide A and Guide B target the start codon and 3′UTR of the SNCA mRNA, respectively. The two guides also contain different features. These features and the sequences of Guide A and Guide B are listed in TABLE 14.

TABLE 14
RNA sequence of exemplary anti-SNCA engineered
polynucleotide to knockdown expression

| SEQ | Engineered | |
|---|---|---|
| ID NO | Polynucleotide | RNA Sequence |
| 71 | Guide A targets | GGUGCUCGCUUCGGCAGCACAU |
| | start site | AUACUUUGUGAAAGAAGGACGG |
| | | GUCACCUUGUCUUUCCUGCUGC |
| | | UUCUGCCACACCCUGUUUGGUC |
| | | UUCUCAGCAGCAGCCACAACUC |
| | | CCUCCUUGGCCUUUGAAAGUCC |
| | | UUUCACGAAUACAUCCA“C”GG |
| | | CUAAUGAAUUCCUUUACACCAC |
| | | ACUGUCGUCGAAUGGCCACUCC |
| | | CAGUUCUCCGCUCACGAGGGUG |
| | | GAAAGGCAGAAGGCUUGAAGGC |
| | | AAGGCGUGAGUGGCCUGUGACU |
| | | AACUGUGCCAAGCGGACUUCGG |
| | | UCCGC |
| 72 | Guide B targets | GAACAUCGUAGAUUGAAGCCAC |
| | 3′UTR | AAAAUCCACAGCACACAAAGAC |
| | | CCUGC“C”ACCAUGUAUUCACU |
| | | UCAGUGAAAGGGAAGCACCGAA |
| | | AUGCUGAGUGGGGGCGUGGAAU |
| | | UUUUGGAGCAGGUUUUCUGACU |
| | | UCGGUCGGAAAACCCCU |

(Italic font denotes U6 protective loops; underlined font denotes quantification tag; underlined and italic font denotes U7smOPT sequence; and “C” denotes the A/C mismatch in the guide)

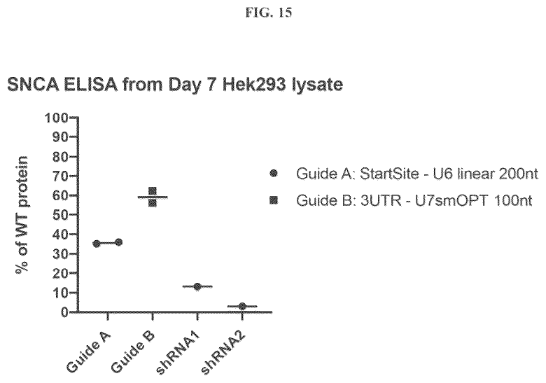

Guide A and Guide B were cloned into a U1 smOpt plasmid. Lysates were prepared 7 days after transfection, and the SNCA protein abundance from the lysate was measured by ELISA. The SNCA protein abundance of the HEK cell lysate without any transfection was used to normalize the SNCA abundance (% of WT protein). As shown by FIG. 15, Guide A and Guide B decreased the abundance of the SNCA protein by about 65% and 40%, respectively. shRNA1 and shRNA2 knocked down the SNCA protein expression by about 90%-99%.

### Example 15: Tiled RNA Editing Against the SNCA mRNA

A gRNA tiling assay was performed to determine different gRNAs lengths and mismatch position, in a first assay focusing at the startsite. The guide RNAs are listed in TABLE 15.

TABLE 15
RNA Sequence of Engineered Polynucleotide Tiled
Across SNCA Gene

| SEQ | |
|---|---|
| ID | |
| NO | RNA Sequence |
| 73 | AGGAGAAGGAGAAGGAGGAGGACUGGGAGGAGGAGGACGGCGAC |
| | GACCAGAAGGGGCCCA |
| 74 | CGACGACCAGAAGGGGCCCAAGAGAGGGGGCGGGCGACCGAGCG |
| | CCGCGACGCGGAAGUG |
| 75 | AGCGCCGCGACGCGGAAGUGAGGUGCGUGCGGGCUGCGGCGCAG |
| | ACCCCGGCCCGGCCCC |
| 76 | GCAGACCCCGGCCCGGCCCCUCCGAGGGCGUCCUGGGCGCUCCC |
| | UCACGCCUUGCCUUCA |
| 77 | UCCCUCACGCCUUGCCUUCAAGCCUUCUGCCUUUCCGCCCUCGU |
| | GAGCGGAGAACUGGGA |
| 78 | UCGUGAGCGGAGAACUGGGAGUGGCCAUUCGACGACGGUGUGGU |
| | GUAAAGGAAUUCAUUA |
| 79 | UGGUGUAAAGGAAUUCAUUAGCCGUGGAUGUAUUCAUGAAAGGA |
| | CUUUCAAAGGCCGAGG |
| 80 | AGGACUUUCAAAGGCCGAGGAGGGAGUUGUGGCUGCUGCUGAGA |
| | AAUCCAAACAGGGUGU |
| 81 | GAGAAAUCCAAACAGGGUGUGGCAGAAGCAGCAGGAAAGACAAA |
| | UGAGGGUGUUCUCUAU |
| 82 | CAAAUGAGGGUGUUCUCUAUGUAGGCUCCGAAACCAAGGAGGGA |
| | GUGGUGCAUGGUGUGG |
| 83 | GGGAGUGGUGCAUGGUGUGGCAACGGUGGCUGAGAAGACCAAAG |
| | AGCAAGUGACAAAUGU |
| 84 | AAAGAGCAAGUGACAAAUGUUGGAGGAGCGGUGGUGACGGGUGU |
| | GACAGCAGUAGCCCAG |
| 85 | GUGUGACAGCAGUAGCCCAGAAGACGGUGGAGGGAGCAGGGAGC |
| | AUUGCAGCAGCCACUG |
| 86 | GAGCAUUGCAGCAGCCACUGGCUUUGUCAAGAAGGACCAGUUGG |
| | GCAAGAAUGAAGAAGG |
| 87 | UUGGGCAAGAAUGAAGAAGGAGCCCCACGGGAAGGAAUUCUGGA |
| | AGAUAUGCCUGUGGAU |
| 88 | UGGAAGAUAUGCCUGUGGAUCCUGACGAUGAGGCUUAUGAAAUG |
| | CCUUCUGAGGAAGGGU |
| 89 | AAUGCCUUCUGAGGAAGGGUAUCAAGACUGCGAACCUGAAGCCU |
| | AAGAAAUAUCUUUGCU |
| 90 | GCCUAAGAAAUAUCUUUGCUCCCGGUUUCUUGAGAUCUGCUGAC |
| | AGAUGUUCCAUCCUGU |
| 91 | UGACAGAUGUUCCAUCCUGUACAAGUGCUCGGUUCCAAUGUGCC |
| | CAGUCAUGACAUUUCU |
| 92 | UGCCCAGUCAUGACAUUUCUCAAAGUUUUUGCAGUGUAUCUCGA |
| | AGUCUUCCAUCAGCAG |
| 93 | UCGAAGUCUUCCAUCAGCAGUGAUUGAAGUGUCUGUACCUGCCC |
| | CCACUCAGCAUUUCGG |
| 94 | GCCCCCACUCAGCAUUUCGGUGCUUCCCUUUCGCUGAAGUGAAU |
| | ACAUGGUAGCAGGGUC |
| 95 | GAAUACAUGGUAGCAGGGUCUUUGUGUGCUGUGGGUUUUGUGGC |
| | UUCAAUCUACGAUGUU |
| 96 | UGGCUUCAAUCUACGAUGUUAAGACAAAUUAAGAACACCUAAGU |
| | GACUACCACUUAUUUC |
| 97 | AAGUGACUACCACUUAUUUCUAAAUCCUCGCUAUUUUUUUGUUG |
| | CUGUUGUUCAGAAGUU |
| 98 | GUUGCUGUUGUUCAGAAGUUGUUGGUGAUUUGCUAUCAUAUAUU |
| | AUAAGAUUUUUAGGUG |
| 99 | UAUUAUAAGAUUUUUAGGUGUCUUUUAAUGAUGCUGUCUAAGAA |
| | UAAUGACGUAUUGUGA |
| 100 | AGAAUAAUGACGUAUUGUGAAAUUUGUUGAUAUAUAUAAUACUU |
| | AAUAAUAUGUGAGCAU |
| 101 | ACUUAAUAAUAUGUGAGCAUGAAACUAUGCGCCUAUAAAUACUA |
| | AAUAUGAAAUUUUACC |
| 102 | ACUAAAUAUGAAAUUUUACCGUUUUGCGAUGUGUUUUAUUCACU |
| | UGUGUUUGUAUAUAAA |
| 103 | CACUUGUGUUUGUAUAUAAAUGGUGAGAAUUAGAAUAAUACGUU |
| | AUCUCAUUGCAUAAAU |
| 104 | CGUUAUCUCAUUGCAUAAAUAUUUUAUUUUUAUCCCGUCUCACU |
| | UUAAUAAUAAUAAUCA |
| 105 | TGGGCCCCTTCTGGTCGTCGCCGTCCTCCTCCTCCCAGTCCTCC |
| | TCCTTCTCCTTCTCCT |
| 106 | CACTTCCGCGTCGCGGCGCTCGGTCGCCCGCCCCCTCTCTTGGG |
| | CCCCTTCTGGTCGTCG |
| 107 | ACCTCACTTCCGCGTCGCGGCGCTCGGTCGCTCGCCCCCTCTCT |
| | TGGGCCCCTTCTGGTC |
| 108 | GGGGCCGGGCCGGGGTCTGCGCCGCAGCCCGCACGCACCTCACT |
| | TCCGCGTCGCGGCGCT |
| 109 | GGACGCTCTCGGAGGGGCCGGGCCGGGGTCTGCGCCGCAGCCCG |
| | CACGCACCTCACTTCC |
| 110 | TGAAGGCAAGGCGTGAGGGAGCGCCCAGGACGCCCTCGGAGGGG |
| | CCGGGCCGGGGTCTGC |
| 111 | TCCCAGTTCTCCGCTCACGAGGGCGGAAAGGCAGAAGGCTTGAA |
| | GGCAAGGCGTGAGGGA |
| 112 | TAATGAATTCCTTTACACCACACCGTCGTCGAATGGCCACTCCC |
| | AGTTCTCCGCTCACGA |
| 113 | CCTCGGCCTTTGAAAGTCCTTTCATGAATACATCCACGGCTAAT |
| | GAATTCCTTTACACCA |
| 114 | ACACCCTGTTTGGATTTCTCAGCAGCAGCCACAACTCCCTCCTC |
| | GGCCTTTGAAAGTCCT |
| 115 | ATAGAGAACACCCTCATTTGTCTTTCCTGCTGCTTCTGCCACAC |
| | CCTGTTTGGATTTCTC |
| 116 | CCACACCATGCACCACTCCCTCCTTGGTTTCGGAGCCTACATAG |
| | AGAACACCCTCATTTG |
| 117 | ACATTTGTCACTTGCTCTTTGGTCTTCTCAGCCACCGTTGCCAC |
| | ACCATGCACCACTCCC |
| 118 | CTGGGCTACTGCTGTCACACCCGTCACCACCGCTCCTCCAACAT |
| | TTGTCACTTGCTCTTT |
| 119 | CAGTGGCTGCTGCAATGCTCCCTGCTCCCTCCACCGTCTTCTGG |
| | GCTACTGCTGTCACAC |
| 120 | CCTTCTTCATTCTTGCCCAACTGGTCCTTCTTGACAAAGCCAGT |
| | GGCTGCTGCAATGCTC |
| 121 | ATCCACAGGCATATCTTCCAGAATTCCTTCCCGTGGGGCTCCTT |
| | CTTCATTCTTGCCCAA |
| 122 | ACCCTTCCTCAGAAGGCATTTCATAAGCCTCATCGTCAGGATCC |
| | ACAGGCATATCTTCCA |
| 123 | AGCAAAGATATTTCTTAGGCTTCAGGTTCGCAGTCTTGATACCC |
| | TTCCTCAGAAGGCATT |
| 124 | ACAGGATGGAACATCTGTCAGCAGATCTCAAGAAACCGGGAGCA |
| | AAGATATTTCTTAGGC |
| 125 | AGAAATGTCATGACTGGGCACATTGGAACCGAGCACTTGTACAG |
| | GATGGAACATCTGTCA |
| 126 | CTGCTGATGGAAGACTTCGAGATACACTGCAAAAACTTTGAGAA |
| | ATGTCATGACTGGGCA |
| 127 | CCGAAATGCTGAGTGGGGGCAGGTACAGACACTTCAATCACTGC |
| | TGATGGAAGACTTCGA |
| 128 | GACCCTGCTACCATGTATTCACTTCAGCGAAAGGGAAGCACCGA |
| | AATGCTGAGTGGGGGC |
| 129 | AACATCGTAGATTGAAGCCACAAAACCCACAGCACACAAAGACC |
| | CTGCTACCATGTATTC |
| 130 | GAAATAAGTGGTAGTCACTTAGGTGTTCTTAATTTGTCTTAACA |
| | TCGTAGATTGAAGCCA |
| 131 | AACTTCTGAACAACAGCAACAAAAAAATAGCGAGGATTTAGAAA |
| | TAAGTGGTAGTCACTT |
| 132 | CACCTAAAAATCTTATAATATATGATAGCAAATCACCAACAACT |
| | TCTGAACAACAGCAAC |
| 133 | TCACAATACGTCATTATTCTTAGACAGCATCATTAAAAGACACC |
| | TAAAAATCTTATAATA |
| 134 | ATGCTCACATATTATTAAGTATTATATATATCAACAAATTTCAC |
| | AATACGTCATTATTCT |
| 135 | GGTAAAATTTCATATTTAGTATTTATAGGCGCATAGTTTCATGC |
| | TCACATATTATTAAGT |
| 136 | TTTATATACAAACACAAGTGAATAAAACACATCGCAAAACGGTA |
| | AAATTTCATATTTAGT |
| 137 | ATTTATGCAATGAGATAACGTATTATTCTAATTCTCACCATTTA |
| | TATACAAACACAAGTG |
| 138 | TGATTATTATTATTAAAGTGAGACGGGATAAAAATAAAATATTT |
| | ATGCAATGAGATAACG |
| 139 | GCTGGGGGAGTGGGAGGCAAACCCGCTAACCCGTCGTCGAATGG |
| | CCACTCCCAGTTCTCC |
| 140 | GCACCAAACTGACATTTGGGGTTTACCTACCCACATAGAGAACA |
| | CCCTCTTGTGTCTTTC |
| 141 | ATCTTTGGATATAAGCACAATGGAGCTTACCCGTTGCCACACCA |
| | TGCACCACTCCCTCCT |
| 142 | AAATGTAACACAAAACGTACACAGCCATACCTTGCCCAACTGGT |
| | CCTTGTTGACAAAGCC |
| 143 | TTGTTAGAAAGATTCAGCTTGGACTCCTACCCCAGAAGGCATTT |
| | CATAAGCCTCATTGTC |
| 144 | ATCCATGGCTAATGAATTCCTTTACACCACACCGGAAAACATAA |
| | AATACACTTTGAATGA |
| 145 | TGCACCACTCCCTCCTTGGTTTTGGAGCCCACAAAAACAAATTC |
| | AAGACATAAGTCTCAA |
| 146 | TTGTCACTTGCTCTTTGGTCTTCTCAGCCACTGGTACAAATAAA |
| | GAGCAACAACAGATTA |
| 147 | ATTCCTTCCTGTGGGGCTCCTTCTTCATTCCAATATTTAAAGTA |
| | AGAAGCACAAAAAGAA |
| 148 | CTTCAGGTTCGTAGTCTTGATACCCTTCCCAATATTAGAAAAAT |
| | CAAAAAGACAGCACAC |

HEK392 cells were transfected with engineered polynucleotides cloned into a Ul smOpt plasmid and SNCA. The sequence of a human U1smOPT plasmid containing gRNA target the SNCA 3′UTR is listed in TABLE 16.

TABLE 16
The sequence of a human U1smOPT plasmid
containing gRNA targeting the SNCA
3′UTR. The guide RNA is
listed in bold.

| SEQ | |
|---|---|
| ID | |
| NO | SEQUENCE |
| 149 | TCGCGCGTTTCGGTGATGACGGTGAAAACCTCTGACACA |
| | TGCAGCTCCCGGAGACGGTCACAGCTTGTCTGTAAGCGG |
| | ATGCCGGGAGCAGACAAGCCCGTCAGGGCGCGTCAGCGG |
| | GTGTTGGCGGGTGTCGGGGCTGGCTTAACTATGCGGCAT |
| | CAGAGCAGATTGTACTGAGAGTGCACCATATGCGGTGTG |
| | AAATACCGCACAGATGCGTAAGGAGAAAATACCGCATCA |
| | GGCGCCATTCGCCATTCAGGCTGCGCAACTGTTGGGAAG |
| | GGCGATCGGTGCGGGCCTCTTCGCTATTACGCCAGCTGG |
| | CGAAAGGGGGATGTGCTGCAAGGCGATTAAGTTGGGTAA |
| | CGCCAGGGTTTTCCCAGTCACGACGTTGTAAAACGACGG |
| | CCAGTGAATTGACGCGCCATTGGGATGTTGTAAAACGAC |
| | GGCCAGTGAACCTGCAGGCAGCTGCGCGCTCGCTCGCTC |
| | ACTGAGGCCGCCCGGGCAAAGCCCGGGCGTCGGGCGACC |
| | TTTGGTCGCCCGGCCTCAGTGAGCGAGCGAGCGCGCAGA |
| | GAGGGAGTGGCCAACTCCATCACTAGGGGTTCCTGCGGC |
| | CGCACGCGTGGAGTAAGGACCAGCTTCTTTGGGAGAGAA |
| | CAGACGCAGGGGCGGGAGGGAAAAAGGGAGAGGCAGACG |
| | TCACTTCCTCTTGGCGACTCTGGCAGCAGATTGGTCGGT |
| | TGAGTGGCAGAAAGGCAGACGGGGACTGGGCAAGGCACT |
| | GTCGGTGACATCACGGACAGGGCGACTTCTATGTAGATG |
| | AGGCAGCGCAGAGGCTGCTGCTTCGCCACTTGCTGCTTC |
| | GCCACGAAGGGAGTTCCCGTGCCCTGGGAGCGGGTTCAG |
| | GACCGCTGATCGGAAGTGAGAATCCCAGCTGTGTGTCAG |
| | GGCTGGAAAGGGCTCGGGAGTGCGCGGGGCAAGTGACCG |
| | TGTGTGTAAAGAGTGAGGCGTATGAGGCTGTGTCGGGGC |
| | AGAGCCCGAAGATCTCACCGAACATCGTAGATCGAAGCC |
| | ACAAAACCCACAGCACACAAAGACCCTGCCACCATGCAT |
| | TCACTTCAGCGAAAGGGAAGCACCGAAATGCCGAGTGGG |
| | GGCGTGGAATTTTTGGAGCAGGTTTTCTGACTTCGGTCG |
| | GAAAACCCCTCCCAATTTCACTGGTCTACAATGAAAGCA |
| | AAACAGTTCTCTTCCCCGCTCCCCGGTGTGTGAGAGGGG |
| | CTTTGATCCTTCTCTGGTTTCCTAGGAAACGCGTATGTG |
| | CTAGCGTACTGAGTCGCCCAGTCTCAGATAGATCCGACG |
| | CCGCCATCTCTAGGCCCGCGCCGGCCCCCTCGCACAGAC |
| | TTGTGGGAGAAGCTCGGCTACTCCCCTGCCCCGGTTAAT |
| | TTGCATATAATATTTCCTAGTAACTATAGAGGCTTAATG |
| | TGCGATAAAAGACAGATAATCTGTTCTTTTTAATACTAG |
| | CTACATTTTACATGATAGGCTTGGATTTCTATAAGAGAT |
| | ACAAATACTAAATTATTATTTTAAAAAACAGCACAAAGG |
| | AAACTCACCCTAACTGTAAAGTAATTGTGTGTTTTGAGA |
| | CTATAAATATCCCTTGGAGAAAAGCCTTGTTTGGAATTC |
| | ATACGCGTTGACATTGATTATTGACTAGTTATTAATAGT |
| | AATCAATTACGGGGTCATTAGTTCATAGCCCATATATGG |
| | AGTTCCGCGTTACATAACTTACGGTAAATGGCCCGCCTG |
| | GCTGACCGCCCAACGACCCCCGCCCATTGACGTCAATAA |
| | TGACGTATGTTCCCATAGTAACGCCAATAGGGACTTTCC |
| | ATTGACGTCAATGGGTGGAGTATTTACGGTAAACTGCCC |
| | ACTTGGCAGTACATCAAGTGTATCATATGCCAAGTACGC |
| | CCCCTATTGACGTCAATGACGGTAAATGGCCCGCCTGGC |
| | ATTATGCCCAGTACATGACCTTATGGGACTTTCCTACTT |
| | GGCAGTACATCTACGTATTAGTCATCGCTATTACCATGG |
| | TGATGCGGTTTTGGCAGTACATCAATGGGCGTGGATAGC |
| | GGTTTGACTCACGGGGATTTCCAAGTCTCCACCCCATTG |
| | ACGTCAATGGGAGTTTGTTTTGGCACCAAAATCAACGGG |
| | ACTTTCCAAAATGTCGTAACAACTCCGCCCCATTGACGC |
| | AAATGGGCGGTAGGCGTGTACGGTGGGAGGTCTATATAA |
| | GCAGAGCTCGTTTAGTGAACCGTCAGATCGCCTGGAGAC |
| | GCCATCCACGCTGTTTTGACCTCCATAGAAGACACCGGG |
| | ACCGATCCAGCCTCCGGACTCTAGAGGATCGAACCCTTA |
| | AGCCGCCACCATGGTGAGCAAGGGCGAGGAGCTGTTCAC |
| | CGGGGTGGTGCCCATCCTGGTCGAGCTGGACGGCGACGT |
| | AAACGGCCACAAGTTCAGCGTGTCCGGCGAGGGCGAGGG |
| | CGATGCCACCTACGGCAAGCTGACCCTGAAGTTCATCTG |
| | CACCACCGGCAAGCTGCCCGTGCCCTGGCCCACCCTCGT |
| | GACCACCCTGACCTACGGCGTGCAGTGCTTCAGCCGCTA |
| | CCCCGACCACATGAAGCAGCACGACTTCTTCAAGTCCGC |
| | CATGCCCGAAGGCTACGTCCAGGAGCGCACCATCTTCTT |
| | CAAGGACGACGGCAACTACAAGACCCGCGCCGAGGTGAA |
| | GTTCGAGGGCGACACCCTGGTGAACCGCATCGAGCTGAA |
| | GGGCATCGACTTCAAGGAGGACGGCAACATCCTGGGGCA |
| | CAAGCTGGAGTACAACTACAACAGCCACAACGTCTATAT |
| | CATGGCCGACAAGCAGAAGAACGGCATCAAGGTGAACTT |
| | CAAGATCCGCCACAACATCGAGGACGGCAGCGTGCAGCT |
| | CGCCGACCACTACCAGCAGAACACCCCCATCGGCGACGG |
| | CCCCGTGCTGCTGCCCGACAACCACTACCTGAGCACCCA |
| | GTCCGCCCTGAGCAAAGACCCCAACGAGAAGCGCGATCA |
| | CATGGTCCTGCTGGAGTTCGTGACCGCCGCCGGGATCAC |
| | TCTCGGCATGGACGAGCTGTACAAGTACTCAGATCTCGA |
| | GCTCAAGTGAACCGGTCAGACATGATAAGATACATTGAT |
| | GAGTTTGGACAAACCACAACTAGAATGCAGTGAAAAAAA |
| | TGCTTTATTTGTGAAATTTGTGATGCTATTGCTTTATTT |
| | GTAACCATTATAAGCTGCAATAAACAAGTTAACAACAAC |
| | AATTGCATTCATTTTATGTTTCAGGTTCAGGGGGAGGTG |
| | TGGGAGGTTTTTTAAACACGTGCGGACCGAGCGGCCGCA |
| | GGAACCCCTAGTGATGGAGTTGGCCACTCCCTCTCTGCG |
| | CGCTCGCTCGCTCACTGAGGCCGGGCGACCAAAGGTCGC |
| | CCGACGCCCGGGCTTTGCCCGGGCGGCCTCAGTGAGCGA |
| | GCGAGCGCGCAGCTGCCTGCAGGCTTGGATCCCAATGGC |
| | GCGCCGAGCTTGGCTCGAGCATGGTCATAGCTGTTTCCT |
| | GTGTGAAATTGTTATCCGCTCACAATTCCACACAACATA |
| | CGAGCCGGAAGCATAAAGTGTAAAGCCTGGGGTGCCTAA |
| | TGAGTGAGCTAACTCACATTAATTGCGTTGCGCTCACTG |
| | CCCGCTTTCCAGTCGGGAAACCTGTCGTGCCAGCTGCAT |
| | TAATGAATCGGCCAACGCGCGGGGAGAGGCGGTTTGCGT |
| | ATTGGGCGCTCTTCCGCTTCCTCGCTCACTGACTCGCTG |
| | CGCTCGGTCGTTCGGCTGCGGCGAGCGGTATCAGCTCAC |
| | TCAAAGGCGGTAATACGGTTATCCACAGAATCAGGGGAT |
| | AACGCAGGAAAGAACATGTGAGCAAAAGGCCAGCAAAAG |
| | GCCAGGAACCGTAAAAAGGCCGCGTTGCTGGCGTTTTTC |
| | CATAGGCTCCGCCCCCCTGACGAGCATCACAAAAATCGA |
| | CGCTCAAGTCAGAGGTGGCGAAACCCGACAGGACTATAA |
| | AGATACCAGGCGTTTCCCCCTGGAAGCTCCCTCGTGCGC |
| | TCTCCTGTTCCGACCCTGCCGCTTACCGGATACCTGTCC |
| | GCCTTTCTCCCTTCGGGAAGCGTGGCGCTTTCTCATAGC |
| | TCACGCTGTAGGTATCTCAGTTCGGTGTAGGTCGTTCGC |
| | TCCAAGCTGGGCTGTGTGCACGAACCCCCCGTTCAGCCC |
| | GACCGCTGCGCCTTATCCGGTAACTATCGTCTTGAGTCC |
| | AACCCGGTAAGACACGACTTATCGCCACTGGCAGCAGCC |
| | ACTGGTAACAGGATTAGCAGAGCGAGGTATGTAGGCGGT |
| | GCTACAGAGTTCTTGAAGTGGTGGCCTAACTACGGCTAC |
| | ACTAGAAGAACAGTATTTGGTATCTGCGCTCTGCTGAAG |
| | CCAGTTACCTTCGGAAAAAGAGTTGGTAGCTCTTGATCC |
| | GGCAAACAAACCACCGCTGGTAGCGGTGGTTTTTTTGTT |
| | TGCAAGCAGCAGATTACGCGCAGAAAAAAAGGATCTCAA |
| | GAAGATCCTTTGATCTTTTCTACGGGGTCTGACGCTCAG |
| | TGGAACGAAAACTCACGTTAAGGGATTTTGGTCATGAGA |
| | TTATCAAAAAGGATCTTCACCTAGATCCTTTTAAATTAA |
| | AAATGAAGTTTTAAATCAATCTAAAGTATATATGAGTAA |
| | ACTTGGTCTGACAGTTAGAAAAACTCATCGAGCATCAAA |
| | TGAAACTGCAATTTATTCATATCAGGATTATCAATACCA |
| | TATTTTTGAAAAAGCCGTTTCTGTAATGAAGGAGAAAAC |
| | TCACCGAGGCAGTTCCATAGGATGGCAAGATCCTGGTAT |
| | CGGTCTGCGATTCCGACTCGTCCAACATCAATACAACCT |
| | ATTAATTTCCCCTCGTCAAAAATAAGGTTATCAAGTGAG |
| | AAATCACCATGAGTGACGACTGAATCCGGTGAGAATGGC |
| | AAAAGTTTATGCATTTCTTTCCAGACTTGTTCAACAGGC |
| | CAGCCATTACGCTCGTCATCAAAATCACTCGCATCAACC |
| | AAACCGTTATTCATTCGTGATTGCGCCTGAGCGAGACGA |
| | AATACGCGATCGCTGTTAAAAGGACAATTACAAACAGGA |
| | ATCGAATGCAACCGGCGCAGGAACACTGCCAGCGCATCA |
| | ACAATATTTTCACCTGAATCAGGATATTCTTCTAATACC |
| | TGGAATGCTGTTTTCCCAGGGATCGCAGTGGTGAGTAAC |
| | CATGCATCATCAGGAGTACGGATAAAATGCTTGATGGTC |
| | GGAAGAGGCATAAATTCCGTCAGCCAGTTTAGTCTGACC |
| | ATCTCATCTGTAACATCATTGGCAACGCTACCTTTGCCA |
| | TGTTTCAGAAACAACTCTGGCGCATCGGGCTTCCCATAC |
| | AATCGATAGATTGTCGCACCTGATTGCCCGACATTATCG |
| | CGAGCCCATTTATACCCATATAAATCAGCATCCATGTTG |
| | GAATTTAATCGCGGCCTAGAGCAAGACGTTTCCCGTTGA |
| | ATATGGCTCATACTCTTCCTTTTTCAATATTATTGAAGC |
| | ATTTATCAGGGTTATTGTCTCATGAGCGGATACATATTT |
| | GAATGTATTTAGAAAAATAAACAAATAGGGGTTCCGCGC |
| | ACATTTCCCCGAAAAGTGCCACCTGACGTCTAAGAAACC |
| | ATTATTATCATGACATTAACCTATAAAAATAGGCGTATC |
| | ACGAGGCCCTTTCGTC |

The cell lysates were prepared 7 days after transfection. ELISA was used to measure the abundance of SNCA protein in the cell lysate. FIG. 15 shows that the engineered polynucleotide Guide A and Guide B decreased the abundance of the mutated SNCA protein by about 65% and 40%, when compared to the wildtype SNCA protein, respectively. In comparison, two shRNAs (shRNA1 and shRNA2) targeting the mutated SNCA protein knocked down its expression by about 90%-99%.

### Example 16: Editing of a Target Adenosine in APP

This example describes editing of a target adenosine in APP using engineered guide RNAs of the present disclosure. Guides were designed to test whether endogenous ADAR could be harnessed to recognize the endogenous APP mRNA in the wildtype and programmed to edit an adenosine two nucleotides away from the clinically relevant adenosine of interest (M671V). The guide sequences targeting the adenosine in APP were designed and are summarized in TABLE 17.

TABLE 17
RNA Sequence of Exemplary anti-APP Engineered
Polynucleotides and their Target RNA (under-
lining indicates the target adenosine)

| | En- | | |
|---|---|---|---|
| SEQ | gineered | Engineered | |
| ID | Polynu- | Polynucleotide | Target RNA |
| NO | cleotide | Sequence | Sequence |
| 159 | 0.100.50 | UUGAUGAUGAACUUCA | ACCAGGUUCUGGGUUGAC |
| | (Exon- | UAUCCUGAGUCAUGUC | AAAUAUCAAGACGGAGGA |
| | Exon) | GGAAUUCUGCAUCCAU | GAUCUCUGAAGUGAAGAU |
| | | CCUCACUUCAGAGAUC | GGAUGCAGAAUUCCGACA |
| | | UCCUCCGUCUUGAUAU | UGACUCAGGAUAUGAAGU |
| | | UUGUCAACCCAGAACC | UCAUCAUCAA |
| | | UGGU | (SEQ ID NO: 150) |
| 160 | 0.100.50 | UUGAUGAUGAACUUCA | UACAGGUUCUGGGUUGAC |
| | (Exon- | UAUCCUGAGUCAUGUC | AAAUAUCAAGACGGAGGA |
| | Intron) | GGAAUUCUGCAUCCAU | GAUCUCUGAAGUGAAGAU |
| | | CCUCACUUCAGAGAUC | GGAUGCAGAAUUCCGACA |
| | | UCCUCCGUCUUGAUAU | UGACUCAGGAUAUGAAGU |
| | | UUGUCAACCCAGAACC | UCAUCAUCAA |
| | | UGUA | (SEQ ID NO: 151) |
| 161 | 0.90.45 | GAUGAACUUCAUAUCC | GUUCUGGGUUGACAAAUA |
| | (Exon | UGAGUCAUGUCGGAAU | UCAAGACGGAGGAGAUCU |
| | only) | UCUGCAUCCAUCCUCA | CUGAAGUGAAGAUGGAUG |
| | | CUUCAGAGAUCUCCUC | CAGAAUUCCGACAUGACU |
| | | CGUCUUGAUAUUUGUC | CAGGAUAUGAAGUUCAUC |
| | | AACCCAGAAC | (SEQ ID NO: 152) |
| 162 | 0.90.45 | ATGATGAACTTCATAT | TCTGGGTTGACAAATATCA |
| | (01) | CCTGAGTCATGTCGGA | AGACGGAGGAGATCTCTG |
| | | ATTCTGCATCCACCTT | AAGTGAAGATGGATGCAG |
| | | CACTTCAGAGATCTCC | AATTCCGACATGACTCAG |
| | | TCCGTCTTGATATTTG | GATATGAAGTTCATCAT |
| | | TCAACCCAGA | (SEQ ID NO: 153) |
| 163 | 0.90.70 | CTTCTGCAAAGAACAC | AGGAGATCTCTGAAGTGA |
| | (02) | CAATTTTTGATGATGA | AGATGGATGCAGAATTCC |
| | | ACTTCATATCCTGAGT | GACATGACTCAGGATATG |
| | | CATGTCGGAATTCTGC | AAGTTCATCATCAAAAATT |
| | | ATCCACCTTCACTTCA | GGTGTTCTTTGCAGAAG |
| | | GAGATCTCCT | (SEQ ID NO: 154) |
| 164 | 0.90.45 | GATGATGAACTTCATA | TCTGGGTTGACAAATATCA |
| | (03) | TCCTGAGTCATGTCGG | AGACGGAGGAGATCTGGA |
| | | AATTCTTCATCCACGT | AGTGAACATGGATGAAGA |
| | | TCACTTCCAGATCTCC | ATTCCGACATGACTCAGG |
| | | TCCGTCTTGATATTTG | ATATGAAGTTCATCATC |
| | | TCAACCCAGA | (SEQ ID NO: 155) |
| 165 | 0.90.70 | TCTTCTGCAAAGAACA | AGGAGATCTGGAAGTGAA |
| | (04) | CCAATTTTTGATGATG | CATGGATGAAGAATTCCG |
| | | AACTTCATATCCTGAG | ACATGACTCAGGATATGA |
| | | TCATGTCGGAATTCTT | AGTTCATCATCAAAAATTG |
| | | CATCCACGTTCACTTC | GTGTTCTTTGCAGAAGA |
| | | CAGATCTCCT | (SEQ ID NO: 156) |
| 166 | 0.60.50 | ATCCTGAGTCATGTCG | ATCAAGACGGAGGAGATC |
| | (05) | GAATTCTTCATCCACG | TGGAAGTGAACATGGATG |
| | | TTCACTTCCAGATCTC | AAGAATTCCGACATGACT |
| | | CTCCGTCTTGAT | CAGGAT |
| | | | (SEQ ID NO: 157) |
| 167 | 0.60.40 | TGAACTTCATATCCTG | AGGAGATCTGGAAGTGAA |
| | (06) | AGTCATGTCGGAATTC | CATGGATGAAGAATTCCG |
| | | TTCATCCACGTTCACT | ACATGACTCAGGATATGA |
| | | TCCAGATCTCCT | AGTTCA |
| | | | (SEQ ID NO: 158) |

Engineered polynucleotides were tested for recruitment and editing of a target adenosine by ADAR in a model cell line and compared to negative controls (a GFP plasmid and a no transfection control). The engineered guide RNAs were designed against pre-mRNA and mRNA, as shown in FIG. 16A. 0.100.50 (Exon-Exon) (SEQ ID NO: 159) is specific to the APP mRNA because it targets the continuous sequence across the exon with the target adenosine and its preceding exon. 0.100.50 (Exon-Intron) (SEQ ID NO: 160) is specific to the APP pre-mRNA because it targets the continuous sequence between the exon with the target adenosine and its preceding intron. 0.90.45 (Exon only) (SEQ ID NO: 161) can target both APP pre-mRNA and mRNA because it only targets the sequence of the target adenosine.

Each engineered polynucleotide was cloned onto a pAAV plasmid backbone (STX-364. The sequence of STX-364 is listed in TABLE 18.

TABLE 18
The complete sequence of STX-364

| SEQ | |
|---|---|
| ID | |
| NO | SEQUENCE |
| 168 | TCGCGCGTTTCGGTGATGACGGTGAAAACCTCTGACACATGC |
| | AGCTCCCGGAGACGGTCACAGCTTGTCTGTAAGCGGATGCCG |
| | GGAGCAGACAAGCCCGTCAGGGCGCGTCAGCGGGTGTTGGCG |
| | GGTGTCGGGGCTGGCTTAACTATGCGGCATCAGAGCAGATTG |
| | TACTGAGAGTGCACCATATGCGGTGTGAAATACCGCACAGAT |
| | GCGTAAGGAGAAAATACCGCATCAGGCGCCATTCGCCATTCA |
| | GGCTGCGCAACTGTTGGGAAGGGCGATCGGTGCGGGCCTCTT |
| | CGCTATTACGCCAGCTGGCGAAAGGGGGATGTGCTGCAAGGC |
| | GATTAAGTTGGGTAACGCCAGGGTTTTCCCAGTCACGACGTT |
| | GTAAAACGACGGCCAGTGAATTGACGCGCCATTGGGATGTTG |
| | TAAAACGACGGCCAGTGAACCTGCAGGCAGCTGCGCGCTCGC |
| | TCGCTCACTGAGGCCGCCCGGGCAAAGCCCGGGCGTCGGGCG |
| | ACCTTTGGTCGCCCGGCCTCAGTGAGCGAGCGAGCGCGCAGA |
| | GAGGGAGTGGCCAACTCCATCACTAGGGGTTCCTGCGGCCGC |
| | ACGCGTGGAGGAGGGCCTATTTCCCATGATTCCTTCATATTT |
| | GCATATACGATACAAGGCTGTTAGAGAGATAATTAGAATTAA |
| | TTTGACTGTAAACACAAAGATATTAGTACAAAATACGTGACG |
| | TAGAAAGTAATAATTTCTTGGGTAGTTTGCAGTTTTAAAATT |
| | ATGTTTTAAAATGGACTATCATATGCTTACCGTAACTTGAAA |
| | GTATTTCGATTTCTTGGCTTTATATATCTTGTGGAAAGGACG |
| | AAACACCGAGAGACCAGCTGGATGGTCTCTTTTTTTTCGTAC |
| | TGAGTCGCCCAGTCTCAGATAGATCCGACGCCGCCATCTCTA |
| | GGCCCGCGCCGGCCCCCTCGCACAGACTTGTGGGAGAAGCTC |
| | GGCTACTCCCCTGCCCCGGTTAATTTGCATATAATATTTCCT |
| | AGTAACTATAGAGGCTTAATGTGCGATAAAAGACAGATAATC |
| | TGTTCTTTTTAATACTAGCTACATTTTACATGATAGGCTTGG |
| | ATTTCTATAAGAGATACAAATACTAAATTATTATTTTAAAAA |
| | ACAGCACAAAAGGAAACTCACCCTAACTGTAAAGTAATTGTG |
| | TGTTTTGAGACTATAAATATCCCTTGGAGAAAAGCCTTGTTT |
| | GGATGTCTTCACAGGAAGACGCTTTTTTTGCGGCCGCATACG |
| | CGTTGACATTGATTATTGACTAGTTATTAATAGTAATCAATT |
| | ACGGGGTCATTAGTTCATAGCCCATATATGGAGTTCCGCGTT |
| | ACATAACTTACGGTAAATGGCCCGCCTGGCTGACCGCCCAAC |
| | GACCCCCGCCCATTGACGTCAATAATGACGTATGTTCCCATA |
| | GTAACGCCAATAGGGACTTTCCATTGACGTCAATGGGTGGAG |
| | TATTTACGGTAAACTGCCCACTTGGCAGTACATCAAGTGTAT |
| | CATATGCCAAGTACGCCCCCTATTGACGTCAATGACGGTAAA |
| | TGGCCCGCCTGGCATTATGCCCAGTACATGACCTTATGGGAC |
| | TTTCCTACTTGGCAGTACATCTACGTATTAGTCATCGCTATT |
| | ACCATGGTGATGCGGTTTTGGCAGTACATCAATGGGCGTGGA |
| | TAGCGGTTTGACTCACGGGGATTTCCAAGTCTCCACCCCATT |
| | GACGTCAATGGGAGTTTGTTTTGGCACCAAAATCAACGGGAC |
| | TTTCCAAAATGTCGTAACAACTCCGCCCCATTGACGCAAATG |
| | GGCGGTAGGCGTGTACGGTGGGAGGTCTATATAAGCAGAGCT |
| | CGTTTAGTGAACCGTCAGATCGCCTGGAGACGCCATCCACGC |
| | TGTTTTGACCTCCATAGTAGACACCGGGACCGATCCAGCCTC |
| | CGGACTCTAGAGGATCGAACCCTTAAGCCGCCACCATGGTGA |
| | GCAAGGGCGAGGAGCTGTTCACCGGGGTGGTGCCCATCCTGG |
| | TCGAGCTGGACGGCGACGTAAACGGCCACAAGTTCAGCGTGT |
| | CCGGCGAGGGCGAGGGCGATGCCACCTACGGCAAGCTGACCC |
| | TGAAGTTCATCTGCACCACCGGCAAGCTGCCCGTGCCCTGGC |
| | CCACCCTCGTGACCACCCTGACCTACGGCGTGCAGTGCTTCA |
| | GCCGCTACCCCGACCACATGAAGCAGCACGACTTCTTCAAGT |
| | CCGCCATGCCCGAAGGCTACGTCCAGGAGCGCACCATCTTCT |
| | TCAAGGACGACGGCAACTACAAGACCCGCGCCGAGGTGAAGT |
| | TCGAGGGCGACACCCTGGTGAACCGCATCGAGCTGAAGGGCA |
| | TCGACTTCAAGGAGGACGGCAACATCCTGGGGCACAAGCTGG |
| | AGTACAACTACAACAGCCACAACGTCTATATCATGGCCGACA |
| | AGCAGAAGAACGGCATCAAGGTGAACTTCAAGATCCGCCACA |
| | ACATCGAGGACGGCAGCGTGCAGCTCGCCGACCACTACCAGC |
| | AGAACACCCCCATCGGCGACGGCCCCGTGCTGCTGCCCGACA |
| | ACCACTACCTGAGCACCCAGTCCGCCCTGAGCAAAGACCCCA |
| | ACGAGAAGCGCGATCACATGGTCCTGCTGGAGTTCGTGACCG |
| | CCGCCGGGATCACTCTCGGCATGGACGAGCTGTACAAGTACT |
| | CAGATCTCGAGCTCAAGTGAACCGGTCAGACATGATAAGATA |
| | CATTGATGAGTTTGGACAAACCACAACTAGAATGCAGTGAAA |
| | AAAATGCTTTATTTGTGAAATTTGTGATGCTATTGCTTTATT |
| | TGTAACCATTATAAGCTGCAATAAACAAGTTAACAACAACAA |
| | TTGCATTCATTTTATGTTTCAGGTTCAGGGGGAGGTGTGGGA |
| | GGTTTTTTAAACACGTGCGGACCGAGCGGCCGCAGGAACCCC |
| | TAGTGATGGAGTTGGCCACTCCCTCTCTGCGCGCTCGCTCGC |
| | TCACTGAGGCCGGGCGACCAAAGGTCGCCCGACGCCCGGGCT |
| | TTGCCCGGGCGGCCTCAGTGAGCGAGCGAGCGCGCAGCTGCC |
| | TGCAGGCTTGGATCCCAATGGCGCGCCGAGCTTGGCTCGAGC |
| | ATGGTCATAGCTGTTTCCTGTGTGAAATTGTTATCCGCTCAC |
| | AATTCCACACAACATACGAGCCGGAAGCATAAAGTGTAAAGC |
| | CTGGGGTGCCTAATGAGTGAGCTAACTCACATTAATTGCGTT |
| | GCGCTCACTGCCCGCTTTCCAGTCGGGAAACCTGTCGTGCCA |
| | GCTGCATTAATGAATCGGCCAACGCGCGGGGAGAGGCGGTTT |
| | GCGTATTGGGCGCTCTTCCGCTTCCTCGCTCACTGACTCGCT |
| | GCGCTCGGTCGTTCGGCTGCGGCGAGCGGTATCAGCTCACTC |
| | AAAGGCGGTAATACGGTTATCCACAGAATCAGGGGATAACGC |
| | AGGAAAGAACATGTGAGCAAAAGGCCAGCAAAAGGCCAGGAA |
| | CCGTAAAAAGGCCGCGTTGCTGGCGTTTTTCCATAGGCTCCG |
| | CCCCCCTGACGAGCATCACAAAAATCGACGCTCAAGTCAGAG |
| | GTGGCGAAACCCGACAGGACTATAAAGATACCAGGCGTTTCC |
| | CCCTGGAAGCTCCCTCGTGCGCTCTCCTGTTCCGACCCTGCC |
| | GCTTACCGGATACCTGTCCGCCTTTCTCCCTTCGGGAAGCGT |
| | GGCGCTTTCTCATAGCTCACGCTGTAGGTATCTCAGTTCGGT |
| | GTAGGTCGTTCGCTCCAAGCTGGGCTGTGTGCACGAACCCCC |
| | CGTTCAGCCCGACCGCTGCGCCTTATCCGGTAACTATCGTCT |
| | TGAGTCCAACCCGGTAAGACACGACTTATCGCCACTGGCAGC |
| | AGCCACTGGTAACAGGATTAGCAGAGCGAGGTATGTAGGCGG |
| | TGCTACAGAGTTCTTGAAGTGGTGGCCTAACTACGGCTACAC |
| | TAGAAGAACAGTATTTGGTATCTGCGCTCTGCTGAAGCCAGT |
| | TACCTTCGGAAAAAGAGTTGGTAGCTCTTGATCCGGCAAACA |
| | AACCACCGCTGGTAGCGGTGGTTTTTTTGTTTGCAAGCAGCA |
| | GATTACGCGCAGAAAAAAAGGATCTCAAGAAGATCCTTTGAT |
| | CTTTTCTACGGGGTCTGACGCTCAGTGGAACGAAAACTCACG |
| | TTAAGGGATTTTGGTCATGAGATTATCAAAAAGGATCTTCAC |
| | CTAGATCCTTTTAAATTAAAAATGAAGTTTTAAATCAATCTA |
| | AAGTATATATGAGTAAACTTGGTCTGACAGTTAGAAAAACTC |
| | ATCGAGCATCAAATGAAACTGCAATTTATTCATATCAGGATT |
| | ATCAATACCATATTTTTGAAAAAGCCGTTTCTGTAATGAAGG |
| | AGAAAACTCACCGAGGCAGTTCCATAGGATGGCAAGATCCTG |
| | GTATCGGTCTGCGATTCCGACTCGTCCAACATCAATACAACC |
| | TATTAATTTCCCCTCGTCAAAAATAAGGTTATCAAGTGAGAA |
| | ATCACCATGAGTGACGACTGAATCCGGTGAGAATGGCAAAAG |
| | TTTATGCATTTCTTTCCAGACTTGTTCAACAGGCCAGCCATT |
| | ACGCTCGTCATCAAAATCACTCGCATCAACCAAACCGTTATT |
| | CATTCGTGATTGCGCCTGAGCGAGACGAAATACGCGATCGCT |
| | GTTAAAAGGACAATTACAAACAGGAATCGAATGCAACCGGCG |
| | CAGGAACACTGCCAGCGCATCAACAATATTTTCACCTGAATC |
| | AGGATATTCTTCTAATACCTGGAATGCTGTTTTCCCAGGGAT |
| | CGCAGTGGTGAGTAACCATGCATCATCAGGAGTACGGATAAA |
| | ATGCTTGATGGTCGGAAGAGGCATAAATTCCGTCAGCCAGTT |
| | TAGTCTGACCATCTCATCTGTAACATCATTGGCAACGCTACC |
| | TTTGCCATGTTTCAGAAACAACTCTGGCGCATCGGGCTTCCC |
| | ATACAATCGATAGATTGTCGCACCTGATTGCCCGACATTATC |
| | GCGAGCCCATTTATACCCATATAAATCAGCATCCATGTTGGA |
| | ATTTAATCGCGGCCTAGAGCAAGACGTTTCCCGTTGAATATG |
| | GCTCATACTCTTCCTTTTTCAATATTATTGAAGCATTTATCA |
| | GGGTTATTGTCTCATGAGCGGATACATATTTGAATGTATTTA |
| | GAAAAATAAACAAATAGGGGTTCCGCGCACATTTCCCCGAAA |
| | AGTGCCACCTGACGTCTAAGAAACCATTATTATCATGACATT |
| | AACCTATAAAAATAGGCGTATCACGAGGCCCTTTCGTC |

About 20,000 WT-HEK293 cells expressing ADAR1 were transfected with 500 ng of each plasmid and the control GFP plasmid. The transfected cells were lysed 48 hours post-transfection. The APP mRNAs were extracted and analyzed by Sanger sequencing. The editing efficiency was quantified using EditR software. As shown in FIG. 16B, about 15-20% editing of the target adenosine in APP was achieved, as compared to less than 3% editing in the negative controls.

### Example 17: Methods for optimizing Engineered Polynucleotide in Human Cells

Provided herein are methods for optimizing engineered polynucleotides in human cells

### A Robust Assay for Screening Guide RNAs

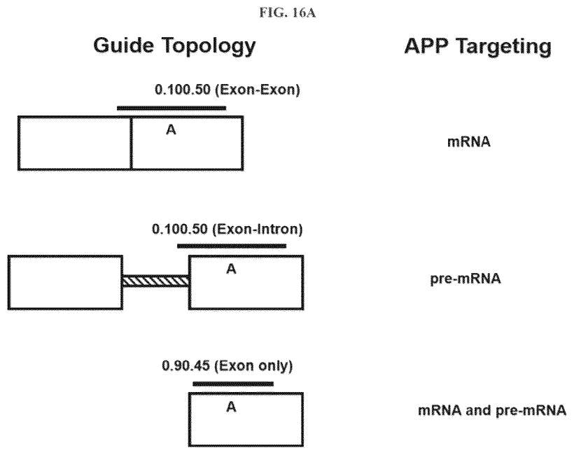

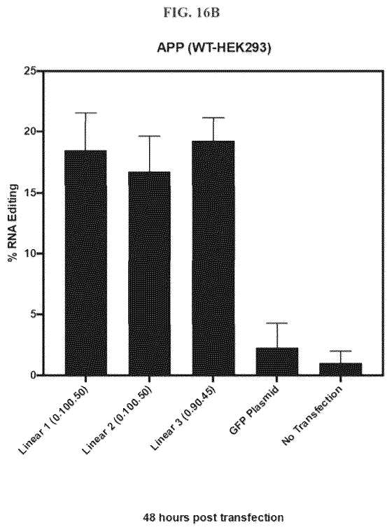

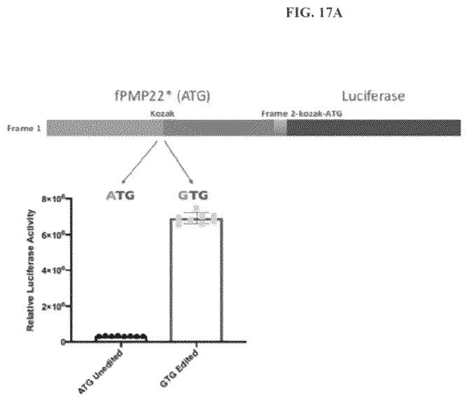

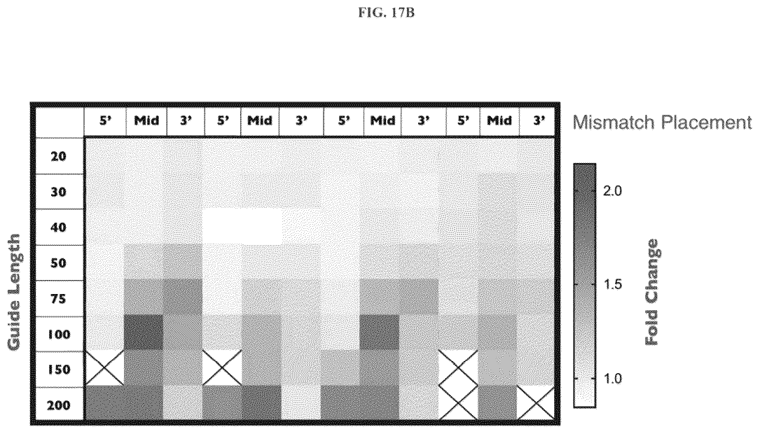

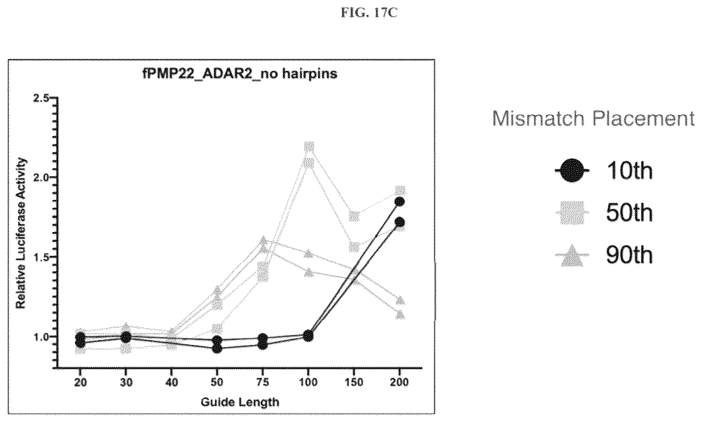

This example describes a robust assay for screening engineered polynucleotide in human cells. A luciferase reporter (for example but not limited to, fPMP22) was developed the RNA editing efficiency of the engineered polynucleotide (or guide). As shown in FIG. 17A, the reporter contains two open reading frames, each with one kozak ATG start codons. The first open reading frame contains a target RNA sequence, while the second one contains a luciferase. When the translation starts from the first ATG start codon, the translation of the luciferase is inhibited. When the target adenosine of the first ATG is mutated to a guanine in which no translation can be initiated, the translation of the luciferase increases. By incorporating the target RNA sequence surrounding the first ATG, the RNA editing efficiencies of the guides with different features-such as the length of the guide, the location of the bulge in the guide, or the location of the GLUR2-recruiting domains in the guide can be tested.

FIG. 17B-FIG. 17C describe the result of a screen using an fPMP22 reporter for use with any one of the compositions or methods provided herein, including but not limited to SNCA, APP, and Tau. FIG. 17B shows a heatmap of the luciferase expression in a fPMP22 reporter. The fPMP22 reporter was generated by inserting the target transcript sequence in Charcot-Marie-Tooth Syndrome 1A into the first open reading frame. The reporter was transposed into HEK293 cells for stable expression. Guides with different lengths (20, 30, 40, 50, 75, 100, 150, and 200 nucleotides); mismatch placement (10th percentile (5′end), 90th percentile (3′ end), or 50th percentile (middle)) of the guide as it's transcribed from 5′ to 3′ from the plasmid were transiently expressed. In some cases, the inclusion/exclusion of specific GLUR2-recruiting domains (0, 1, or 2) and their location (5′ end, 3′ end, both or neither) can also be tested. The fold-change of the luciferase expression normalized to that of cells transfected with plasmids with non-specific guides and ADAR2 (ST0145). The sequences of these guides are listed in TABLE 19.

TABLE 19
Sequences of the guide RNAs used in the
fPMP22 reporter screen.

| SEQ | | Per- | |
|---|---|---|---|
| ID | | cen- | |
| NO | Length | tile | Sequence |
| 169 | 20 | 10 | ACTCTGGCGGCAAGTTCTGC |
| 170 | 30 | 10 | CACTCTGGCGGCAAGTTCTGCTCAGCGGAG |
| 171 | 40 | 10 | GCACTCTGGCGGCAAGTTCTGCTCAGCGGAGTTTC |
| | | | TGCCC |
| 172 | 50 | 10 | AGCACTCTGGCGGCAAGTTCTGCTCAGCGGAGTTT |
| | | | CTGCCCGGCCAAACA |
| 173 | 75 | 10 | AGGAGCACTCTGGCGGCAAGTTCTGCTCAGCGGA |
| | | | GTTTCTGCCCGGCCAAACAGCGTAACCCCTTCTTC |
| | | | CAAGCA |
| 174 | 100 | 10 | GGAGGAGCACTCTGGCGGCAAGTTCTGCTCAGCG |
| | | | GAGTTTCTGCCCGGCCAAACAGCGTAACCCCTTCT |
| | | | TCCAAGCAGATTTCTTTGCAGCCAAATGCAA |
| 175 | 150 | 10 | CAACAGGAGGAGCACTCTGGCGGCAAGTTCTGCT |
| | | | CAGCGGAGTTTCTGCCCGGCCAAACAGCGTAACC |
| | | | CCTTCTTCCAAGCAGATTTCTTTGCAGCCAAATGC |
| | | | AAGGGATGTTAAGGCAAGACCCTCCCCACAGGGC |
| | | | AGTCAGAGACCCG |
| 176 | 200 | 10 | CTCAGCAACAGGAGGAGCACTCTGGCGGCAAGTT |
| | | | CTGCTCAGCGGAGTTTCTGCCCGGCCAAACAGCG |
| | | | TAACCCCTTCTTCCAAGCAGATTTCTTTGCAGCCA |
| | | | AATGCAAGGGATGTTAAGGCAAGACCCTCCCCAC |
| | | | AGGGCAGTCAGAGACCCGCAGCCGACAGACTAAG |
| | | | CCTGCAGCTTCCAACCAGGCTCCCCGAGA |
| 177 | 20 | 50 | AGGAGGAGCACTCTGGCGGC |
| 178 | 30 | 50 | GCAACAGGAGGAGCACTCTGGCGGCAAGTT |
| 179 | 40 | 50 | ACTCAGCAACAGGAGGAGCACTCTGGCGGCAAGT |
| | | | TCTGCT |
| 180 | 50 | 50 | ATGATACTCAGCAACAGGAGGAGCACTCTGGCGG |
| | | | CAAGTTCTGCTCAGCG |
| 181 | 75 | 50 | ACGTGGAGGACGATGATACTCAGCAACAGGAGGA |
| | | | GCACTCTGGCGGCAAGTTCTGCTCAGCGGAGTTTC |
| | | | TGCCCG |
| 182 | 100 | 50 | CACCAGCACCGCGACGTGGAGGACGATGATACTC |
| | | | AGCAACAGGAGGAGCACTCTGGCGGCAAGTTCTG |
| | | | CTCAGCGGAGTTTCTGCCCGGCCAAACAGCGT |
| 183 | 150 | 50 | TGACGATCGTGGAGACGAACAGCAGCACCAGCAC |
| | | | CGCGACGTGGAGGACGATGATACTCAGCAACAGG |
| | | | AGGAGCACTCTGGCGGCAAGTTCTGCTCAGCGGA |
| | | | GTTTCTGCCCGGCCAAACAGCGTAACCCCTTCTTC |
| | | | CAAGCAGATTTCT |
| 184 | 200 | 50 | TGTCCATTGCCCACGATCCATTGGCTGACGATCGT |
| | | | GGAGACGAACAGCAGCACCAGCACCGCGACGTG |
| | | | GAGGACGATGATACTCAGCAACAGGAGGAGCACT |
| | | | CTGGCGGCAAGTTCTGCTCAGCGGAGTTTCTGCCC |
| | | | GGCCAAACAGCGTAACCCCTTCTTCCAAGCAGAT |
| | | | TTCTTTGCAGCCAAATGCAAGGGATGTTA |
| 185 | 20 | 90 | TCAGCAACAGGAGGAGCACT |
| 186 | 30 | 90 | CGATGATACTCAGCAACAGGAGGAGCACTC |
| 187 | 40 | 90 | CGTGGAGGACGATGATACTCAGCAACAGGAGGAG |
| | | | CACTCT |
| 188 | 50 | 90 | GCACCGCGACGTGGAGGACGATGATACTCAGCAA |
| | | | CAGGAGGAGCACTCTG |
| 189 | 75 | 90 | GTGGAGACGAACAGCAGCACCAGCACCGCGACGT |
| | | | GGAGGACGATGATACTCAGCAACAGGAGGAGCA |
| | | | CTCTGGCG |
| 190 | 100 | 90 | CCACGATCCATTGGCTGACGATCGTGGAGACGAA |
| | | | CAGCAGCACCAGCACCGCGACGTGGAGGACGATG |
| | | | ATACTCAGCAACAGGAGGAGCACTCTGGCGGC |
| 191 | 150 | 90 | AGAGGTGCTACAGTTCTGCCAGAGATCAGTTGCG |
| | | | TGTCCATTGCCCACGATCCATTGGCTGACGATCGT |
| | | | GGAGACGAACAGCAGCACCAGCACCGCGACGTG |
| | | | GAGGACGATGATACTCAGCAACAGGAGGAGCACT |
| | | | CTGGCGGCAAGTTC |

˜50,000 HEK293 cells expressing the reporter transcript were transfected with 300 ng of each of the above guide expressing plasmids in biological duplicate. Twenty-four hours post transfection, supernatant was collected and assessed for luciferase activity. Measurements of absorbance after incubation with luciferase substrate were taken on the varioskan lux and normalized against a plasmid expressing a non-specific guide to control for plasmid and/or ADAR expression.

FIG. 17C shows a line graph of the relationship of the guide length (x-axis) and the fold-change of the reporter expression (y-axis) in two biological replicates for each guide. The result of 3 sets of experiments, in which the guide contained a GLUR2-recruiting domain in the 3′ end of the guide and the location of the bulge was varied, was shown.

### Cell Line Development

Different cells lines were engineered to examine RNA editing.

Endogenous ADAR1 was modified to examine its function in RNA editing. FIG. 18A shows the knockout strategy of the ADAR1 locus. Two gRNAs US gRNA and DS gRNA were designed to cover a 6 kb region of the ADAR1 locus, encompassing the deaminase domain (789^{th} to 1221^{st} amino acid). A K562 cell line was transfected with the gRNAs and a homology directed repair (HDR) oligo with 80 bp homology arms outside the 6 kb region. The gRNAs nicked the DNA strands. The HDR oligo would create the 6 kb deletion in the ADAR1 locus through the homologous recombination repair pathway, removing the deaminase domain. FIG. 18B shows a western blot of ADAR 1 in different clones transfected with US and DS gRNA. GAPDH was used as a control. Two clones #9 and #11 showed no detectable ADAR1 protein expression by the western blot.

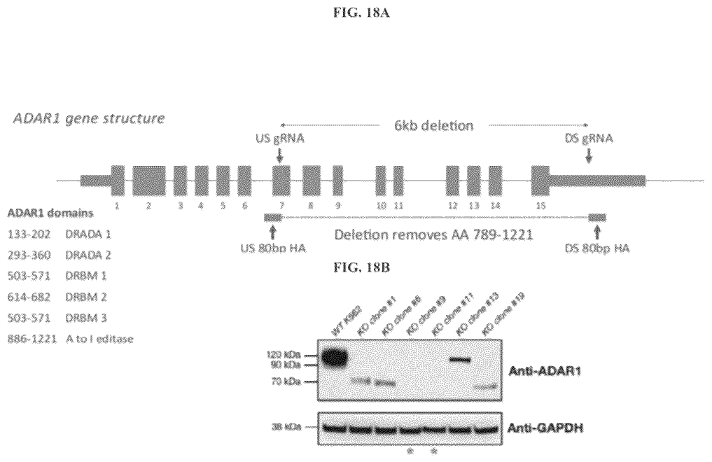

Endogenous ADAR2 was modified to examine its function in RNA editing. FIG. 19A shows the knockout strategy of the ADAR2 locus. Two gRNAs US gRNA and DS gRNA were designed to cover a 9.5 kb region of the ADAR1 locus, encompassing the deaminase domain (70^{th} to 522^{nd} amino acid). A K562 cell line was transfected with the gRNAs and a homology directed repair (HDR) oligo with 80 bp homology arms outside the 9.5 kb region. The gRNAs nicked the DNA strands. The HDR oligo would create the 9.5 kb deletion in the ADAR2 locus through the homologous recombination repair pathway, removing the deaminase domain.

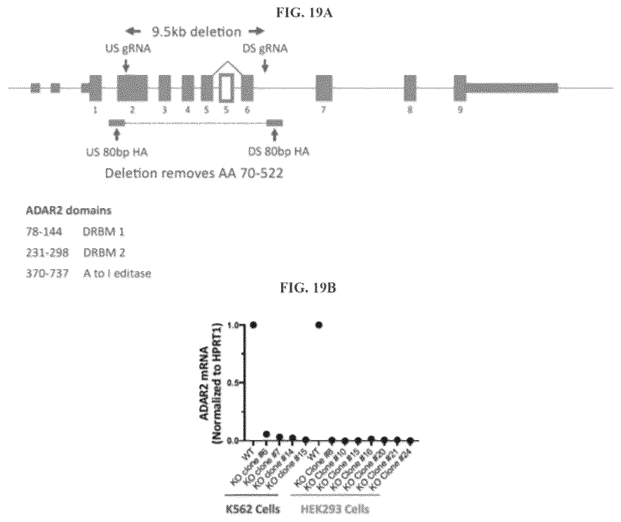

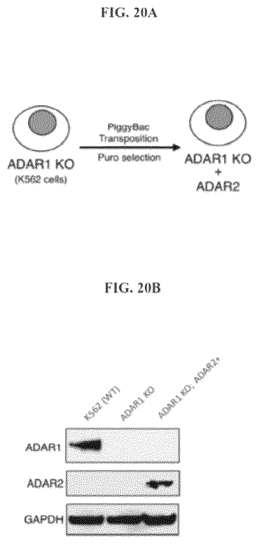

A cell line that expresses ADAR2 but not ADAR1 was generated. FIG. 20A shows the strategy to generate the ADAR1 knockout (KO) cell line that overexpresses ADAR2. An ADAR2 overexpression construct, maintained as a PiggyBac transposon with a puromycin-resistant marker, was transfected and integrated into an ADAR1 KO K562 cell line. The successfully integrated cell was selected by puromycin resistance. FIG. 20B shows a western blot of ADAR1 and ADAR2 protein expression in wildtype, ADAR1 KO, and ADAR1 KO+ADAR2 cell. GAPDH was used as a control. The wildtype or ADAR1 KO cell did not express ADAR2. Only the ADAR1 KO cell successfully integrated with ADAR2 OE construct expressed ADAR2.

### An Assay to Measure RNA Editing

An assay utilizing digital droplet PCR and fluorescence quantification to measure RNA editing efficiencies was developed.

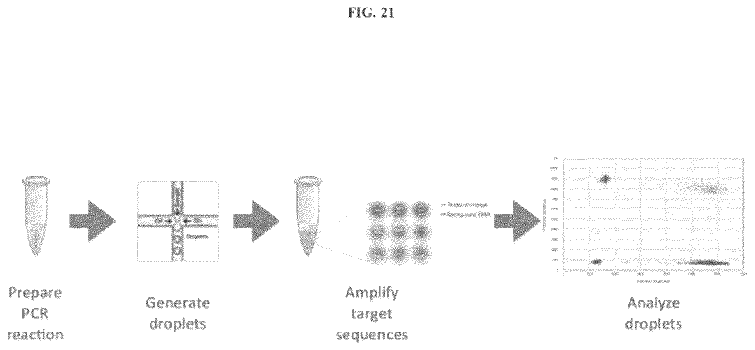

FIG. 21 shows the general scheme of a Bio-Rad Drop-off digital droplet PCR (ddPCR) assay to measure RNA editing efficiency. PCR samples prepared and then processed on fluidic chips to generate droplets of PCR reactions in oil suspension. The target and background reference sequences are detected: 1, by PCR amplification with intercalating fluorescent dyes; or 2, by fluorescent TaqMan style probes on the PCR amplified products. The resultant signal is analyzed by a droplet reader. Data is then presented in a two-dimensional dot plot, showing high and low populations of droplets for each fluorescent channel.

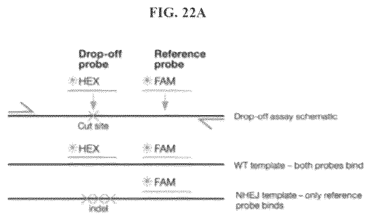

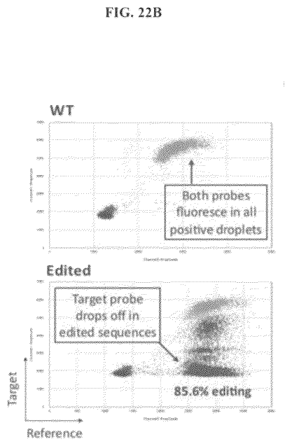

FIG. 22 shows the design of ddPCR Drop-off assay probes. A forward and reverse primer were designed to flank the Rab7 mRNA (A genomic locus can also be targeted in the case of measuring DNA editing). A Drop-off probe and reference TaqMan probe was designed to bind a target site in Rab7 and the region adjacent to the target site, respectively. Both probes could bind the wildtype sequence of the target site and the adjacent site to release signals; the Drop-off probe could not bind an edited or mutated sequence on the target site to release the signal. Each Rab7A mRNA molecule was converted to a cDNA molecule by reverse transcription and PCR amplification and allocated into individual droplet. The percentage of the populations of the edited vs wildtype Rab7 mRNA molecule, measured by the ddPCR, was counted to determine the editing frequency. FIG. 22B shows the result of this experiment: In the wildtype control (WT) sample with no editing, most droplets showed high fluorescent intensity for both probes. In the edited sample, about 85% of the droplets showed decreased fluorescent intensity in the Drop-off probe, suggesting that an equivalent percentage of sequences were edited.

An Assay to Quantify gRNA Level

An assay to quantify gRNA level was developed.

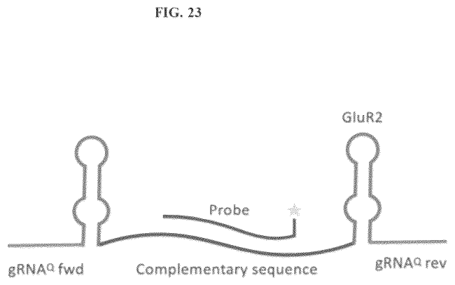

FIG. 23 shows the design of a pair of universal gRNA quantification (gRNAQ) tags to quantify gRNA abundance. They are added to the 5′ and 3′ ends of a guide RNA. The universal sequences allow for detection of any guide RNA inserted between the tags with addition of a guide specific TaqMan probe. In qPCR or ddPCR, the primers will bind the gRNAQ tags for amplification. The guide specific TaqMan probe will the produce a fluorescent signal that can be quantified using a standard curve with qPCR or ddPCR to measure gRNA abundance.

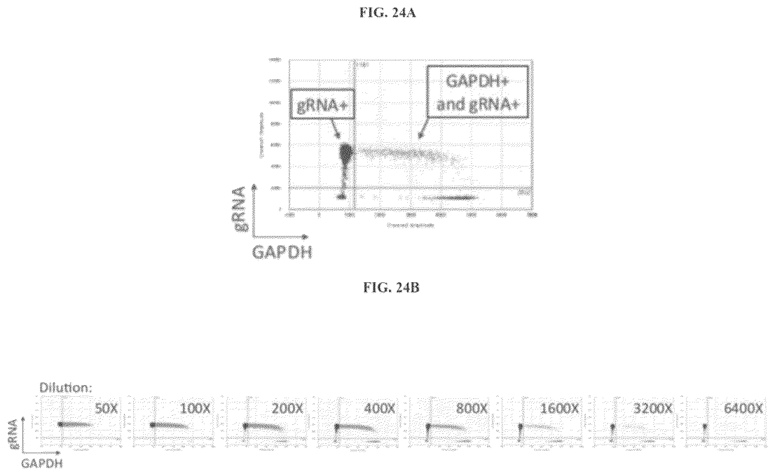

FIG. 24A shows the result of the quantification of gRNA targeting Rab7 with gRNA^{Q} tag. GRAPH mRNA was used as a control. The total number of positive droplets for Rab7 gRNA and GAPGH were counted. Assuming a Poisson distribution, the frequency of all events was determined.

Since a highly abundant target could use up all the amplification reagents, the less abundant targets might not be able to be allocated with enough amplification agents; one get-around is to dilute the sample so that every target, even one in a minute amount, is adequately amplified. FIG. 24B shows the dot plots demonstrating that multiple serial dilutions (50×, 100×, 200×, 400×, 800×, 1600×, 3200×, 6400×) of the sample to obtain amplification of GAPDH mRNA and Rab7 gRNA in the sample. As GAPDH mRNA was diluted out with increasing dilution factors, more Rab7 gRNA-positive droplets were identified.

gRNA Structure

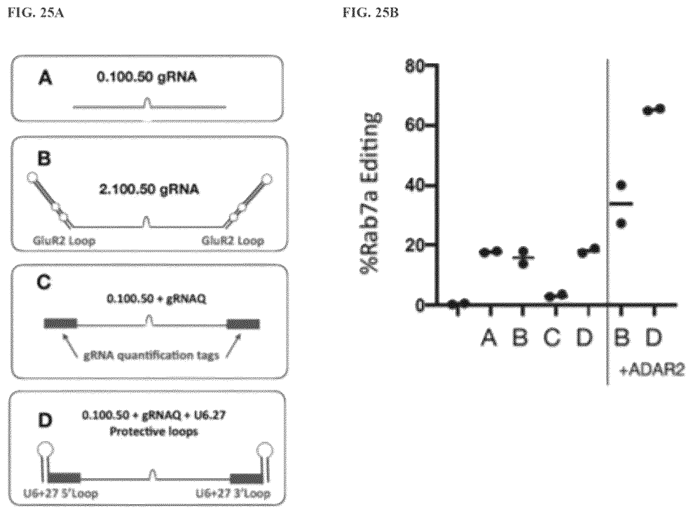

As shown in FIGS. 25A and 25B, the gRNA with protective loops at both ends of the gRNA could increase the RNA editing efficiency of the gRNA. Cells were transfected with different gRNAs and vectors expressing ADAR2. Cells transfected with vectors that did not express ADAR2 were used as controls. After at least two days of transfection, cells were lysed, and Rab7a RNA editing efficiency was analyzed by Sanger sequencing, as shown in Example 13, 14, and 18. Two biological replicates were provided.

Optimizing gRNA Expression

The expression of guide RNAs was optimized to enhance RNA editing efficiency.

gRNA targeting Rab7 was driven by CMV enhancers. Two configurations of the CMV enhancer were oriented against the hU6 promoter in several of the constructs and listed in TABLE 20. The constructs were designed to co-express ADAR2 or GFP as well as two GluRD domains on the 5′ and 3′ ends of the guide targeting Rab7a. The guides used is 100 nt long with the A/C mismatch placed at the 50th base position. The guide sequence used is listed in SEQ ID NO: 192.

SEQ ID NO: 192:
TGATAAAAGGCGTACATAATTCTTGTGTCTACTGTACAGAATACTGCCG
CCAGCTGGATTTCCCAATTCTGAGTAACACTCTGCAATCCAAACAGGGT
TC.

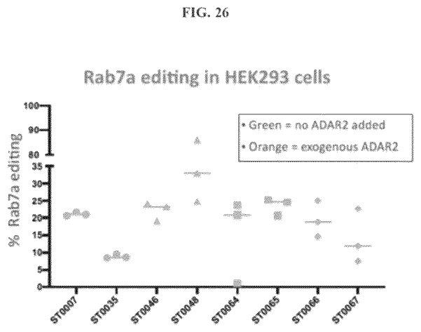

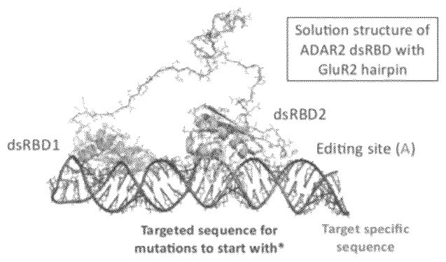

˜20,000 HEK293 cells were transfected with 1 μg of plasmid expressing SEQ ID NO: 192. Total RNA was collected 48 hours post transfection. Three biological replicates were tested. The editing results are shown in FIG. 26. The features of each experiment are listed in TABLE 20.

TABLE 20
Construct used in FIG. 26.

| Construct # | Construction Description |
|---|---|
| ST0007 | hu6_2_100_50_GFP |
| ST0035 | ADAR2 only, no gRNA |
| ST0046 | hu6_2_100_50_2 bulge_GFP |
| ST0048 | hu6_2_100_50_2 bulge_ADAR2 |
| ST0064 | xiaCMVen-hu6_2_100_50_2 bulge_GFP |
| ST0065 | xiaCMVen-hu6_2_100_50_2 bulge_ADAR2 |
| ST0066 | sgCMVen-hu6_2_100_50_2 bulge_GFP |
| ST0067 | sgCMVen-hu6_2_100_50_2 bulge_ADAR2 |

Different promoters, such as human U6 (hu6), xiaCMVen-hu6, and sgCMVen-hu6 promoters were used to drive the expression of gRNA against Rab7a. The gRNA comprises two bulges. When no gRNA was provided (ST0035), RNA editing was at the minimum. Co-expression of ADAR2, when the gRNA is expressed by hu6, boosts the RNA editing efficiency.

### Design

The 3-dimensional solution structure of ADAR2 binding to the gRNA/target RNA complex is shown in FIG. 27. The structure shows the location of the contact of the two dsRBD (dsRBD 1 and 2) binding to the GluR2 hairpin of the gRNA/target RNA complex. The target editing site adenosine is located close to dsRBD2. The structure of gRNA or ADAR2 proteins can be rationally optimized based on the contacts of the solution structure.

### Example 18: Platforms for Processing RNA Editing Data

Provided herein are platforms for platforms for processing RNA editing data.

EditR is an algorithm to process and analyze RNA editing data. It is available as an R package or online through Shiny. Sequencing data, either with Sanger or Next-Generation Sequencing, can be analyzed with EditR.

For example, as in the A-to-I mRNA editing in converting a G to an A, three parameters of the sequence are specified in EditR: 1, the sequence, as in the Sanger file format (.ab1), is loaded into the program platform; 2, the region of interest is specified, usually the entire target region of the gRNA used in the experiment; 3, the sequence is specified as the target or reverse complement sequence.

EditR then assess the sequence for potential editing across the specified target region. Using the peak height and area under the curve for each base at each position, it predicts the percentage of each base at each position. The frequency of A and G in the target residue is then determined.

### Example 19: RNA Hyper-Editing

Provided herein are methods for RNA hyper-editing.

In some cases, editing more than one nucleotide can be advantageous. This type of editing, as referred herein as hyper-editing, can generate an edited RNA molecule with more than one nucleotide changes. The nucleotides can lead to more than one amino acid substitution in some cases. The nucleotides may not lead to any amino acid substitutions in other cases. Either way, the hyper-edited RNA and the modified polypeptide encoded by it can have advantageous therapeutic potential. For example, multiple nucleotide substitutions in the 5′UTR and coding sequences of an mRNA can create artificial kozak start codons or and eliminate the endogenous one. In this case, the translational frame of an mRNA is disrupted. Multiple nucleotide substitutions in the 3′UTR can disrupt sequences important for RNA stability or transport, such as the poly-A tail of an mRNA, diminishing its ability to be translated. Multiple nucleotide substitutions in the coding sequence can confer multiple amino acid substitutions. These changes can affect the protein function, such as the target sites by processing enzymes. One example is the post-translational processing of secretory or transmembrane proteins, usually involving multiple enzymes and cleavages (and glycosylation).

Hyper-editing can be achieved by designing a guide RNA with multiple mis-matches to the target region; for example, the gRNA listed in TABLE 16 is used to create hyper-editing of the 3′UTR of the SNCA mRNA. An example of creating multiple amino acid substitution with multiple nucleotide substitutions in the coding sequence of an mRNA is shown in Example 1. SEQ ID NO: 51 is used to create multiple amino acid changes that lead that impact the APP processing by BACE1, according to TABLE 8.

Hyper-editing can also be achieved by the design of the elements of the guide RNA, other than those base-pairing with the target region; for example, a guide RNA can have an RNA editing entity recruiting domain may not release the RNA editing entity once engaged, forcing multiple editing on the target RNA.

### Example 20: Additional Assays to Measure Effects of RNA Editing

Provided herein are methods to measure various biological effects of RNA editing. These methods can apply to any target RNA or the polypeptide encoded by the target RNA. Such target RNA can include the APP, SNCA, or Tau mRNA or pre-mRNA.

RNA editing can affect the abundance of the target RNA. It can create nucleotide substitutions that decrease the target mRNA abundance. To measure such effect, using Tau as an illustrative example, a cell is transfected with a gRNA targeting the target mRNA. At least 48 hours after transfection, the mRNA of the cells can be prepared, and the Tau mRNA can be measured by Q-PCR.

RNA editing can affect the abundance of a polypeptide of encoded by the target RNA. To measure such effect, using Tau as an illustrative example, a cell is transfected with a gRNA targeting the target mRNA. At least 48 hours after transfection, the protein lysate of the cells can be prepared, and the Tau polypeptide can be measured by western blot using a Tau-specific antibody. If an antibody is not available, one can co-transfect the cell with a vector that comprises a nucleic acid that expresses a fusion Tau mRNA that encodes a Tau polypeptide with an affinity tag. Such affinity can be FLAG, HIS, HA, MYC, or any other affinity tags known in the art. The abundance of the tagged Tau polypeptide, reflecting the RNA editing efficiency, can be traced and measured using the affinity tag and western blot.

RNA editing can affect the enzymatic processing such as cleavage of a polypeptide of encoded by the target RNA. To measure such effect, using Tau as an illustrative example, a cell is transfected with a gRNA targeting the target mRNA. At least 48 hours after transfection, the protein lysate of the cells can be prepared, and the Tau polypeptide can be measured by western blot using a Tau-specific antibody. If an antibody is not available, one can co-transfect the cell with a vector that comprises a nucleic acid that expresses a fusion Tau mRNA that encodes a Tau polypeptide with an affinity tag. Such affinity can be FLAG, HIS, HA, MYC, or any other affinity tags known in the art. The enzymatic processing such as cleavage of the tagged Tau polypeptide, reflecting the RNA editing efficiency, can be traced and measured using the affinity tag and western blot.

RNA editing can affect the phosphorylation status of a polypeptide of encoded by the target RNA. To measure such effect, using Tau as an illustrative example, a cell is transfected with a gRNA targeting the target mRNA. At least 48 hours after transfection, the protein lysate of the cells can be prepared, and the Tau polypeptide can be measured by western blot using Tau-specific antibody. If an antibody is not available, one can co-transfect the cell with a vector that comprises a nucleic acid that expresses a fusion Tau mRNA that encodes a Tau polypeptide with an affinity tag. Such affinity can be FLAG, HIS, HA, MYC, or any other affinity tags known in the art. The phosphorylation of the tagged Tau polypeptide, reflecting the RNA editing efficiency, can be traced and measured using the affinity tag and western blot. In these cases, the molecular weight of the Tau polypeptide is affected by the phosphorylation status of the polypeptide. Usually a polypeptide with more phosphorylation migrates slower than one with fewer phosphorylation. This phenomenon is also known in the art as gel-shift.

RNA editing can affect the aggregation status of a polypeptide of encoded by the target RNA. To measure such effect, using Tau as an illustrative example, a cell is transfected with a gRNA targeting the target mRNA. At least 48 hours after transfection, the protein lysate of the cells can be prepared, and the Tau polypeptide can be measured by western blot using a Tau-specific antibody. If an antibody is not available, one can co-transfect the cell with a vector that comprises a nucleic acid that expresses a fusion Tau mRNA that encodes a Tau polypeptide with an affinity tag. Such affinity can be FLAG, HIS, HA, MYC, or any other affinity tags known in the art. The aggregation status of the tagged Tau polypeptide, reflecting the RNA editing efficiency, can be traced and measured using the affinity tag and western blot. Aggregated Tau polypeptide is detected with a large gel-shift.

The phosphorylation status of a polypeptide can also be measured by a phosphorylation site specific, phosphorylation fragment specific, or phosphorylation polypeptide specific antibody. In this case, only a phosphorylated Tau polypeptide is detected by the antibody

The phosphorylation status of a polypeptide is also measured by mass-spectroscopy. An endogenous or epitope tagged proteins are immunopurified (IP) from cell lysates, purified via gel electrophoresis or precipitation and enzymatically digested into peptides. Samples can be optionally enriched for phosphopeptides using immobilized metal affinity chromatography (IMAC) or titanium dioxide (TiO2) and then analyzed by microcapillary liquid chromatography/tandem mass spectrometry (LC-MS/MS).

The ability of a polypeptide to be phosphorylated is also measured by an in vitro phosphorylation assay. In vitro kinase assays are often performed using radioactive ³²P- or ³³P-labeled ATP. The transfer of the gamma phosphate from ATP (or sometimes GTP) to a substrate, Tau in this example, is measured. A protein is expressed and purified. The purified protein is incubated with a kinase and ³²P- or ³³P-labeled ATP. When a polypeptide is phosphorylated, it is radioactively labelled by the radioactive ATP. When this protein is run in a western blot, an autoradiograph is used to measure the amount of radioactive ATP, representing the amount of phosphorylation on the polypeptide during the in vitro kinase assay.

### EMBODIMENTS

Embodiment 1 An engineered polynucleotide that comprises a targeting sequence that is at least partially complementary to a region of a target RNA, wherein the region of the target RNA: (a) at least partially encodes for: an amyloid precursor protein (APP) polypeptide, an alpha-synuclein (SNCA) polypeptide, or a Tau polypeptide; (b) comprises a sequence that is proximal to (a); or (c) comprises (a) and (b); wherein the engineered polynucleotide is configured to: facilitate an editing of a base of a nucleotide of a polynucleotide in the region of the target RNA by an RNA editing entity; facilitate a modulation of the expression of the APP polypeptide, the SNCA polypeptide, or the Tau polypeptide; or a combination thereof.

Embodiment 2 The engineered polynucleotide of embodiment 1, wherein by the facilitating the editing of the base of the nucleotide of the polynucleotide in the region of the target RNA by the RNA editing entity, the engineered polynucleotide is configured to facilitate modulation of processing and/or cleavage of the target RNA by a secretase enzyme.

Embodiment 3 The engineered polynucleotide of embodiment 2, wherein the target RNA is the APP polypeptide.

Embodiment 4 The engineered polynucleotide of any one of embodiments 1-3, wherein the region of the target RNA is cleaved by a secretase enzyme.

Embodiment 5 The engineered polynucleotide of embodiment 4, wherein the secretase is: a beta secretase; a γ-secretase; or a beta secretase and a γ-secretase.

Embodiment 6 The engineered polynucleotide of embodiment 5, comprising the beta secretase, and wherein the beta secretase comprises beta-site amyloid precursor protein cleaving enzyme 1, cathepsin B, or Meprin beta.

Embodiment 7 The engineered polynucleotide of any one of embodiments 1-6, comprising (b), wherein the sequence that is proximal to the region of the target RNA at least partially encoding the APP polypeptide, the SNCA polypeptide, or the Tau polypeptide comprises at least a portion of a three prime untranslated region (3′ UTR).

Embodiment 8 The engineered polynucleotide of any one of embodiments 1-7, comprising (b), wherein the sequence that is proximal to the region of the target RNA at least partially encoding the APP polypeptide, the SNCA polypeptide, or the Tau polypeptide comprises at least a portion of a five prime untranslated region (5′ UTR).

Embodiment 9 The engineered polynucleotide of embodiment 8, wherein the editing of a base of the 5′UTR results in at least partially regulating gene translation of the APP polypeptide, the SNCA polypeptide, or the Tau polypeptide.

Embodiment 10 The engineered polynucleotide of embodiment 8, wherein the editing of the base of the nucleotide of the polynucleotide of the region of the 5′UTR results in facilitating regulating mRNA translation of the APP.

Embodiment 11 The engineered polynucleotide of any one of embodiments 1-10, comprising (b), wherein the sequence that is proximal to the region of the target RNA at least partially encoding the APP polypeptide, the SNCA polypeptide, or the Tau polypeptide comprises at least a portion of: a poly(A) tail, microRNA response element (MRE), AU-rich element (ARE), or any combination thereof.

Embodiment 12 The engineered polynucleotide of any one of embodiments 1-11, wherein the region of the target RNA at least partially encodes for the APP polypeptide.

Embodiment 13 The engineered polynucleotide of any one of embodiments 1-11, wherein the region of the target RNA at least partially encodes for the SNCA polypeptide.

Embodiment 14 The engineered polynucleotide of any one of embodiments 1-11, wherein the region of the target RNA at least partially encodes for the Tau polypeptide.

Embodiment 15 The engineered polynucleotide of embodiment 6, wherein the engineered polynucleotide is configured to facilitate the cleavage of the target RNA by the beta-site amyloid precursor protein cleaving enzyme 1.

Embodiment 16 The engineered polynucleotide of any one of embodiments 1-15, wherein the engineered polynucleotide is configured to facilitate the editing of the base of the nucleotide of the polynucleotide of the region of the target RNA by the RNA editing entity.

Embodiment 17 The engineered polynucleotide of any one of embodiments 1-16, wherein the targeting sequence that is at least partially complementary to the region of the target RNA comprises at least one nucleotide that is not complementary to a nucleotide in the region of the target RNA.

Embodiment 18 The engineered polynucleotide of embodiment 17, wherein the at least one nucleotide that is not complementary is an adenosine (A), and wherein the A is comprised in an A/C mismatch.

Embodiment 19 The engineered polynucleotide of any one of embodiments 1-18, wherein the target RNA is selected from the group comprising: a mRNA, a tRNA, a lncRNA, a lincRNA, a miRNA, a rRNA, a snRNA, a microRNA, a siRNA, a piRNA, a snoRNA, a snRNA, a exRNA, a scaRNA, a YRNA, and a hnRNA.

Embodiment 20 The engineered polynucleotide of embodiment 19, wherein the target RNA is the mRNA.

Embodiment 21 The engineered polynucleotide of any one of embodiments 1-20, wherein the region of the target RNA comprises a mutation as compared to an otherwise comparable region encoding a wildtype APP polypeptide, a wildtype SNCA polypeptide, or a wildtype Tau polypeptide.

Embodiment 22 The engineered polynucleotide of embodiment 21, wherein the mutation comprises a polymorphism.

Embodiment 23 The engineered polynucleotide of any one of embodiments 1-22, wherein the targeting sequence is about: 40, 60, 80, 100, or 120 nucleotides in length.

Embodiment 24 The engineered polynucleotide of embodiment 23, wherein the targeting sequence is about 100 nucleotides in length.

Embodiment 25 The engineered polynucleotide of any one of embodiments 1-24, wherein the engineered polynucleotide further comprises an RNA editing entity recruiting domain that is capable of recruiting the RNA editing entity.

Embodiment 26 The engineered polynucleotide of embodiment 25, wherein the RNA editing entity recruiting domain is at least 1 to about 75 nucleotides in length.

Embodiment 27 The engineered polynucleotide of embodiment 26, wherein the RNA editing entity recruiting domain is at least 30-50 nucleotides in length.

Embodiment 28 The engineered polynucleotide of any one of embodiments 25-27, wherein the RNA editing entity recruiting domain comprises a glutamate ionotropic receptor AMPA type subunit 2 (GluR2) sequence.

Embodiment 29 The engineered polynucleotide of embodiment 28, wherein the GluR2 sequence comprises at least about 80%, 85%, 90%, 95%, or 99% sequence identity to SEQ ID NO:

1.

Embodiment 30 The engineered polynucleotide of embodiment 29, wherein the GluR2 sequences comprises SEQ ID NO: 1.

Embodiment 31 The engineered polynucleotide of any one of embodiments 1-30, wherein the RNA editing entity comprises an adenosine deaminase acting on RNA (ADAR) polypeptide or biologically active fragment thereof or adenosine deaminase acting on tRNA (ADAT) polypeptide or biologically active fragment thereof.

Embodiment 32 The engineered polynucleotide of embodiment 31, comprising the ADAR polypeptide or biologically active fragment thereof, which comprises ADAR1 or ADAR2.

Embodiment 33 The engineered polynucleotide of any one of embodiments 1-24, wherein the engineered polynucleotide lacks a recruiting domain.

Embodiment 34 The engineered polynucleotide of any one of embodiments 1-33, wherein the engineered polynucleotide further comprises a structural feature which at least in part recruits an RNA editing entity.

Embodiment 35 The engineered polynucleotide of embodiment 34, wherein the structural feature comprises: a bulge, a hairpin, an internal loop, a structured motif, and any combination thereof.

Embodiment 36 The engineered polynucleotide of embodiment 35, wherein the structural feature comprises the bulge.

Embodiment 37 The engineered polynucleotide of embodiment 36, wherein the bulge is an asymmetric bulge.

Embodiment 38 The engineered polynucleotide of embodiment 36, wherein the bulge is a symmetric bulge.

Embodiment 39 The engineered polynucleotide of any one of embodiments 36-38, wherein the bulge is from 1-29 nucleotides in length.

Embodiment 40 The engineered polynucleotide of embodiment 35, wherein the structural feature comprises the hairpin.

Embodiment 41 The engineered polynucleotide of embodiment 35, wherein the structural feature comprises the internal loop.

Embodiment 42 The engineered polynucleotide of embodiment 41, wherein the internal loop is an asymmetric loop.

Embodiment 43 The engineered polynucleotide of embodiment 41, wherein the internal loop is a symmetric loop.

Embodiment 44 The engineered polynucleotide of embodiment 35, wherein the structural feature comprises the structured motif.

Embodiment 45 The engineered polynucleotide of embodiment 44, wherein the structured motif comprises at least two of: the bulge, the hairpin, and the internal loop.

Embodiment 46 The engineered polynucleotide of embodiment 45, wherein the structured motif comprises the bulge and the hairpin.

Embodiment 47 The engineered polynucleotide of embodiment 45, wherein the structured motif comprises the bulge and the internal loop.

Embodiment 48 The engineered polynucleotide of any one of embodiments 1-47, wherein the engineered polynucleotide comprises a backbone that comprises a plurality of sugar and phosphate moieties covalently linked together, and wherein the backbone comprises a 5′ reducing hydroxyl, a 3′ reducing hydroxyl, or both.

Embodiment 49 The engineered polynucleotide of embodiment 48, wherein each of the 5′ reducing hydroxyl in the backbone is linked to each of the 3′ reducing hydroxyl via a phosphodiester bond.

Embodiment 50 The engineered polynucleotide of any one of embodiments 1-47, wherein the engineered polynucleotide comprises a backbone that comprises a plurality of sugar and phosphate moieties covalently linked together, and wherein the backbone lacks a 5′ reducing hydroxyl, a 3′ reducing hydroxyl, or both.

Embodiment 51 The engineered polynucleotide of any one of embodiments 1-50, wherein when the engineered polynucleotide associates with the region of the target RNA, the association comprises hybridized polynucleotide strands.

Embodiment 52 The engineered polynucleotide of embodiment 51, wherein the hybridized polynucleotide strands at least in part form a duplex.

Embodiment 53 The engineered polynucleotide of any one of embodiments 1-52, wherein the engineered polynucleotide further comprises a chemical modification.

Embodiment 54 The engineered polynucleotide of any one of embodiments 1-53, wherein the engineered polynucleotide comprises RNA, DNA, or both.

Embodiment 55 The engineered polynucleotide of embodiment 54, wherein the engineered polynucleotide comprises the RNA.

Embodiment 56 An engineered polynucleotide configured to facilitate an editing of a base of a nucleotide of a polynucleotide of a region of a target RNA at least partially encoding an amyloid precursor protein (APP), wherein an RNA editing entity, in association with the engineered polynucleotide and the target RNA, edits the base of the nucleotide of the polynucleotide of the region of the target RNA, wherein the editing results in generation of an edited target RNA at least partially encoding a modified amyloid precursor protein (APP).

Embodiment 57 The engineered polynucleotide of embodiment 56, wherein the RNA editing entity comprises a secretase enzyme.

Embodiment 58 The engineered polynucleotide of embodiment 57, wherein the secretase enzyme is beta secretase; a γ-secretase; or a beta secretase and a γ-secretase.

Embodiment 59 The engineered polynucleotide of embodiment 58, wherein the secretase enzyme is the beta secretase, and wherein the beta secretase is selected from the group consisting of: beta-site amyloid precursor protein cleaving enzyme 1, cathepsin B, and Meprin beta.

Embodiment 60 An engineered polynucleotide configured to facilitate, by an RNA editing entity, an editing of a base of a nucleotide of a polynucleotide of a region of a target RNA at least partially encoding an amyloid precursor protein (APP), wherein the editing results in generation of an edited target RNA that comprises at least one amino acid substitution compared to an otherwise comparable unedited target RNA, wherein the edited target RNA encodes an APP with an altered susceptibility to a beta secretase cleavage compared to the otherwise comparable APP encoded by the otherwise comparable unedited target RNA; and wherein a cell expressing an APP polypeptide generated from the edited target RNA has substantially no decrease in beta secretase activity on an endogenous substrate of beta secretase compared to a corresponding cell expressing an APP polypeptide generated from the unedited target RNA, as determined by an in vitro assay comprising a measurement of a metabolite indicative of cleavage of the endogenous substrate by beta-site amyloid precursor protein cleaving enzyme 1 (BACE1), and wherein the endogenous substrate comprises amyloid-like protein 1 (APLP1), amyloid-like protein 2 (APLP2), Contactin 2, Jagged 1, neural cell adhesion molecule L1 (CHL1), Neurexin 1α, Neurexin 3β, neuregulin 1 (NRG1), seizure related protein 6 (SEZ6), seizure related protein 6 precursor protein (SEZ6L), a β (β1-4) Auxiliary subunit of the voltage-gated sodium ion channel (VGSC) subtype Nav1, VGSC Accessory Subunits KCNE1 or KCNE2, a functional portion of any of these, or any combination of thereof.

Embodiment 61 The engineered polynucleotide of embodiment 60, wherein the beta secretase comprises BACE1, cathepsin B, or Meprin beta.

Embodiment 62 The engineered polynucleotide of embodiment 60, wherein the endogenous substrate comprises the NRG1, the SEZ6, or the CHL1.

Embodiment 63 An engineered polynucleotide configured to facilitate, by an RNA editing entity, an editing of a base of a nucleotide of a polynucleotide of a region of a target RNA at least partially encoding an amyloid precursor protein (APP), wherein the editing results in generation of a modified APP encoded by an edited target RNA that comprises at least one amino acid substitution compared to an otherwise comparable unmodified APP encoded by an comparable unedited target RNA, and wherein the modified APP polypeptide generated from the edited target RNA: (i) produces a lower amount of Abeta40, Abeta42, or both when expressed in a cell as compared to an APP polypeptide generated from the unedited target RNA as measured by an Abeta40 or Abeta42 enzyme linked immunosorbent assay (ELISA); (ii) produces an increased amount of secreted ectodomain APP alpha (sAPPa) when expressed in a cell as compared to the sAPPa generated from the unedited target RNA as measured by an sAPPa ELISA; or (iii) any combination of (i) and (ii).

Embodiment 64 A vector that comprises: (a) the engineered polynucleotide of any one of embodiments 1-63, (b) a polynucleotide encoding the engineered polynucleotide of any one of embodiments 1-63; or (c) (a) and (b).

Embodiment 65 The vector of embodiment 64, further comprising a second engineered polynucleotide or a second polynucleotide encoding the second engineered polynucleotide.

Embodiment 66 The vector of embodiment 65, wherein the engineered polynucleotide and the second engineered polynucleotide are the same.

Embodiment 67 The vector of embodiment 65, wherein the engineered polynucleotide and the second engineered polynucleotide are different.

Embodiment 68 The vector of any one of embodiments 64-67, wherein the second engineered polynucleotide comprises a second targeting sequence that at least partially hybridizes to a region of a second target RNA.

Embodiment 69 The vector of any one of embodiments 65-68, wherein the second engineered polynucleotide comprises an siRNA, an shRNA, an miRNA, a piRNA, an anti-sense oligonucleotide; or does not comprise at least one of these.

Embodiment 70 The vector of any one of embodiments 65-69, wherein the engineered polynucleotide and the second engineered polynucleotide are contiguous with each other.

Embodiment 71 The vector of any one of embodiments 65-70, wherein the polynucleotide of the vector independently encodes: the engineered polynucleotide and the second engineered polynucleotide, are operatively linked to a same promoter sequence.

Embodiment 72 The vector of any one of embodiments 65-70, wherein the engineered polynucleotide and the second engineered polynucleotide not contiguous with each other.

Embodiment 73 The vector of any one of embodiments 65-72, wherein the engineered polynucleotide comprises a targeting sequence that is at least partially complementary to a region of the APP target RNA, and wherein the second engineered polynucleotide comprises a targeting sequence that is at least partially complementary to a region of the SNCA or the Tau target RNA.

Embodiment 74 The vector of any one of embodiments 65-73, wherein the engineered polynucleotide comprises a targeting sequence that is at least partially complementary to a region of the APP target RNA, and wherein the second engineered polynucleotide comprises the siRNA, the shRNA, the miRNA, the piRNA, or the anti-sense oligonucleotide that targets the SNCA polypeptide or the Tau polypeptide.

Embodiment 75 The vector of any one of embodiments 64-74, wherein the vector is a viral vector.

Embodiment 76 The vector of embodiment 75, wherein the viral vector is an AAV vector, and wherein the AAV vector is of a serotype selected from the group comprising: AAV2, AAV5, AAV6, AAV8, AAV9, a portion thereof, a fusion product thereof, and any combination thereof.

Embodiment 77 The vector of embodiment 76, wherein the AAV vector comprises rep and ITR sequences from AAV2 and a cap sequence from AAV5.

Embodiment 78 The vector of any one of embodiments 76-77, wherein the AAV vector comprises an ITR sequence that is a self-complementary ITR.

Embodiment 79 The vector of any one of embodiments 76-78, wherein the AAV vector that encodes for the engineered polynucleotide is self-complementary.

Embodiment 80 A pharmaceutical composition in unit dose form comprising the engineered polynucleotide of any one of embodiments 1-63 or the vector of any one of embodiments 64-79.

Embodiment 81 The pharmaceutical composition in unit dose form of embodiment 80, further comprising a pharmaceutically acceptable: excipient, carrier, or diluent.

Embodiment 82 A method of making a pharmaceutical composition comprising admixing the engineered polynucleotide of any one of embodiment 1-63 with a pharmaceutically acceptable excipient, diluent, or carrier.

Embodiment 83 An isolated cell comprising the engineered polynucleotide of any one of embodiments 1-63, the vector of any one of embodiments 64-79, or both.

Embodiment 84 A kit comprising the engineered polynucleotide of any one of embodiments 1-63, the vector of any one of embodiments 64-79, or both in a container.

Embodiment 85 A method of making a kit comprising inserting the engineered polynucleotide of any one of embodiments 1-63 into a container.

Embodiment 86 A method of treating or preventing a disease or condition in a subject in need thereof, the method comprising administering to a subject in need thereof: (a) the vector of any one of embodiments 64-79; (b) the pharmaceutical composition of any one of embodiments 80-81; or (c) (a) and (b).

Embodiment 87 A method of treating or preventing a disease or condition comprising administering a therapeutic to a subject in need thereof, wherein the therapeutic facilitates, by an RNA editing entity, an editing of a base of a nucleotide of a polynucleotide of a region of a target RNA that at least partially encodes for an amyloid precursor protein (APP), thereby generating an edited RNA that at least partially encodes for a beta secretase-resistant APP as compared to an otherwise comparable APP encoded by an otherwise comparable RNA lacking the edit as determined by in vitro assay comprising contacting the beta secretase-resistant APP and the otherwise comparable APP with: a) a beta secretase; b) a γ-secretase; c) or a beta secretase and a γ-secretase.

Embodiment 88 The method of embodiment 87, wherein the beta secretase comprises beta-site amyloid precursor protein cleaving enzyme 1, cathepsin B, or Meprin beta.

Embodiment 89 The method of embodiment 88, wherein the therapeutic comprises a vector comprising or encoding an engineered polynucleotide that comprises a targeting sequence that at least partially hybridizes to a region of the target RNA.

Embodiment 90 The method of any one of embodiments 87-89, wherein the engineered polynucleotide further comprises an RNA editing entity recruiting domain that is capable of recruiting the RNA editing entity.

Embodiment 91 The method of embodiment 90, wherein the RNA editing entity recruiting domain is at least 1 to about 75 nucleotides in length.

Embodiment 92 The method of any one of embodiments 90-91, wherein the RNA editing entity recruiting domain comprises a glutamate ionotropic receptor AMPA type subunit 2 (GluR2) sequence.

Embodiment 93 The method of any one of embodiments 87-92, wherein the RNA editing entity comprises an adenosine deaminase acting on RNA (ADAR) polypeptide or biologically active fragment thereof or adenosine deaminase acting on tRNA (ADAT) polypeptide or biologically active fragment thereof.

Embodiment 94 The method of embodiment 93, wherein the RNA editing entity comprises the ADAR polypeptide or biologically active fragment thereof, and wherein the ADAR comprises ADAR1 or ADAR2.

Embodiment 95 The method of any one of embodiments 87-89, wherein the engineered polynucleotide lacks an RNA editing entity recruiting sequence.

Embodiment 96 The method of any one of embodiments 87-95, wherein the engineered polynucleotide further comprises a structural feature.

Embodiment 97 The method of embodiment 96, wherein the structural feature comprises: a bulge, a hairpin, an internal loop, a structured motif, and any combination thereof.

Embodiment 98 The method of embodiment 97, wherein the structural feature comprises the bulge.

Embodiment 99 The method of embodiment 98, wherein the bulge is an asymmetric bulge. Embodiment 100 The method of embodiment 98, wherein the bulge is a symmetric bulge.

Embodiment 101 The method of any one of embodiments 98-100, wherein the bulge is from 1-29 nucleotides in length.

Embodiment 102 The method of embodiment 97, wherein the structural feature comprises the hairpin.

Embodiment 103 The method of embodiment 97, wherein the structural feature comprises the internal loop.

Embodiment 104 The method of embodiment 103, wherein the internal loop is asymmetric.

Embodiment 105 The method of embodiment 103, wherein the internal loop is asymmetric.

Embodiment 106 The method of embodiment 97, wherein the structural feature comprises the structured motif.

Embodiment 107 The method of embodiment 97, wherein the structured motif comprises at least two of: the bulge, the hairpin, and the internal loop.

Embodiment 108 The method of embodiment 107, wherein the structured motif comprises the bulge and the hairpin.

Embodiment 109 The method of embodiment 107, wherein the structured motif comprises the bulge and the internal loop.

Embodiment 110 The method of any one of embodiments 87-109, wherein the engineered polynucleotide comprises a backbone that comprises a plurality of sugar and phosphate moieties covalently linked together, and wherein the backbone comprises a 5′ reducing hydroxyl, a 3′ reducing hydroxyl, or both.

Embodiment 111 The engineered polynucleotide of embodiment 110, wherein each of the 5′ reducing hydroxyl in the backbone is linked to each of the 3′ reducing hydroxyl via a phosphodiester bond.

Embodiment 112 The engineered polynucleotide of any one of embodiments 87-109, wherein the engineered polynucleotide comprises a backbone that comprises a plurality of sugar and phosphate moieties covalently linked together, and wherein the backbone lacks a 5′ reducing hydroxyl, a 3′ reducing hydroxyl, or both.

Embodiment 113 The method of any one of embodiments 87-112, wherein the beta secretase-resistant APP has reduced susceptibility to cleavage at a position cleavable by a beta secretase as compared to the otherwise comparable APP produced from the otherwise comparable RNA lacking the edit.

Embodiment 114 The method of embodiment 113, wherein the beta secretase comprises BACE1, cathepsin B, or Meprin beta.

Embodiment 115 The method of any one of embodiments 87-114, wherein the nucleotide is comprised in a codon which encodes an amino acid in proximity to a cleavage site of the APP.

Embodiment 116 The method of embodiment 115, wherein the cleavage site at the APP is selected from the group consisting of: an α-secretase cleavage site, a β-secretase cleavage site, a β′-secretase cleavage site, a γ-secretase cleavage site, and any combination thereof.

Embodiment 117 The method of any one of embodiments 87-116, wherein the amino acid is at position 669, 670, 671, 672, 673, 682, 683, 684, 687, 688, 711, 712, 713, or 714 of the APP of SEQ ID NO: 2.

Embodiment 118 The method of any one of embodiments 87-117, wherein the BACE protease-resistant APP comprises at least one amino acid residue difference as compared to the otherwise comparable APP produced from the otherwise comparable RNA lacking the edit.

Embodiment 119 The method of embodiment 118, wherein the one amino acid residue difference comprises an amino acid substitution that results in a change in charge, hydrophobicity, or polarity of the amino acid, or any combination thereof.

Embodiment 120 The method of any one of embodiments 118-119, wherein the difference in the amino acid comprises a conservative substitution.

Embodiment 121 The method of any one of embodiments 118-119, wherein the difference in the amino acid comprises a charge neutral substitution.

Embodiment 122 The method of any one of embodiments 118-119, wherein the difference in the amino acid residue comprises a K to E change, a K to R change, a K to G change, an M to V change, a D to G change, an E to G change, an H to R change, or any combination thereof.

Embodiment 123 The method of embodiment 122, wherein the difference in the amino acid residue comprises K670E, K670R, K670G, M671V, D672G, E682G, H684R, K687R, K687E, or K687G of the amyloid precursor protein of SEQ ID NO: 2.

Embodiment 124 The method of embodiment 122 or 123, wherein the change in the one amino acid comprises K670G or M671V of the amyloid precursor protein of SEQ ID NO: 2.

Embodiment 125 The method of any one of embodiments 87-124, wherein the target RNA is selected from the group comprising: an mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA.

Embodiment 126 The method of embodiment 125, wherein the target RNA is the mRNA.

Embodiment 127 The method of any one of embodiments 87-125, wherein the therapeutic directly facilitates the edit.

Embodiment 128 The method of any one of embodiments 87-125, wherein the therapeutic indirectly facilitates the edit.

Embodiment 129 The method of any one of embodiments 87-128, wherein the disease or condition comprises a neurodegenerative disease or condition.

Embodiment 130 The method of embodiment 129, wherein the neurodegenerative condition comprises Alzheimer's disease, Parkinson's disease, dementia, Lewy Body Dementia, progressive supranuclear palsy, frontotemporal lobar degeneration, corticobasal degeneration, or any combination thereof.

Embodiment 131 The method of any one of embodiments 87-128, wherein the condition comprises traumatic brain injury, Down's syndrome, cancer, Fragile X Syndrome, autism, amyotrophic lateral sclerosis, multiple sclerosis, Lesch-Nyhan disease, metabolic disorder, or any combination thereof.

Embodiment 132 The method of any one of embodiments 87-131, wherein the edited RNA or the BACE protease-resistant APP is generated in at least 5%, 8%, 10%, 15%, 20%, 30%, 40%, or 50% of the subjects administered the therapeutic in a clinical trial.

Embodiment 133 The method of any one of embodiments 87-132, further comprising a second administering of an additional therapeutic agent.

Embodiment 134 The method of embodiment 133, wherein the administering and the second administering are consecutive.

Embodiment 135 The method of embodiment 133, wherein the administering and the second administering are concurrent.

Embodiment 136 The method of any one of embodiments 133-135, wherein the administering or the second administering or both are independently repeated at least once a week.

Embodiment 137 The method of any one of embodiments 133-136, wherein the administering or the second administering or both are independently performed by parenteral route of administration.

Embodiment 138 The method of any one of embodiments 133-137, wherein the administering or the second administering or both are independently performed by parenchymal injection, intra-thecal injection, intra-ventricular injection, intra-cisternal injection, intravenous injection, or intranasal administration or any combination thereof.

1. A composition that comprises: an engineered polynucleotide comprising a targeting sequence that is at least partially complementary to a region of a target RNA, wherein the region of the target RNA:
(a) comprises a sequence that at least partially encodes for an amyloid precursor protein (APP) polypeptide;
(b) comprises a sequence that is proximal to (a); or
(c) comprises (a) and (b); and wherein the engineered polynucleotide is configured to facilitate an editing of a base of a nucleotide of the target RNA by an RNA editing entity.

2. The composition of claim 1, wherein the editing of the base of the nucleotide of the target RNA by the RNA editing entity facilitates an increase or a decrease of:
(a) a processing;
(b) a cleavage; or
(c) (a) and (b),
of the APP polypeptide by a secretase enzyme, relative to an APP polypeptide encoded by the target RNA without the editing.

3. The composition of claim 2, wherein the secretase enzyme comprises: an alpha secretase; a beta secretase; a gamma secretase; or a combination thereof.

4. The composition of claim 3, wherein the secretase enzyme comprises the beta secretase, and wherein the beta secretase comprises beta-site amyloid precursor protein cleaving enzyme 1 (BACE1), cathepsin B, or Meprin beta.

5.-32. (canceled)

33. The composition of claim 1, wherein the nucleotide is comprised in a codon which encodes an amino acid in proximity to a cleavage site of the APP polypeptide, and wherein the amino acid is at position 670, 671, 672, 673, 682, 684, 687, 712, or 714 of the APP polypeptide comprising the polypeptide sequence of SEQ ID NO: 2.

34.-85. (canceled)

86. An engineered polynucleotide that comprises a sequence that comprises at least 90%, 95%, 97%, or 99% sequence identity with at least a portion of a sequence selected from: SEQ ID NO: 52, SEQ ID NO: 71-SEQ ID NO: 148, and SEQ ID NO: 159-SEQ ID NO: 167.

87. An engineered polynucleotide that comprises a targeting sequence capable of at least partially binding to a sequence that comprises at least 90%, 95%, 97%, or 99% sequence identity with a portion of a sequence selected from: SEQ ID NO: 150-SEQ ID NO: 158 as determined by BLAST.
